SEQUENCE LISTING (148 sequences)

SEQ ID NO: 1 - protein, 416 aa, Lachancea kluyveri
MSAVTVTGSDPKNRGSSSNTEQEVPKVAIDTNGNVFSVPDFTIKDILGAIPHECYERRLATSLYYVFRDIFCMLTTGYLTHKILYPLLISYTSNSIIKFTFWALYTYVQGLFGTGIWVLAHECGHQAFSDYGIVNDFVGWTLHSYLMVPYFSWKYSHGKHHKATGHMTRDMVFVPATKEEFKKSRNFFGNLAEYSEDSPLRTLYELLVQQLGGWIAYLFVNVTGQPYPDVPSWKWNHFWLTSPLFEQRDALYIFLSDLGILTQGIVLTLWYKKFGGWSLFINWFVPYIWVNHWLVFITFLQHTDPTMPHYNAEEWTFAKGAAATIDRKFGFIGPHIFHDIIETHVLHHYCSRIPFYNARPASEAIKKVMGKHYRSSDENMWKSLWKSFRSCQYVDGDNGVLMFRNINNCGVGAAEK

SEQ ID NO: 2 - protein, 419 aa, Yarrowia lipolytica
MDSTTQTNTGTGKVAVQPPTAFIKPIEKVSEPVYDTFGNEFTPPDYSIKDILDAIPQECYKRSYVKSYSYVARDCFFIAVFAYMAYAYLPLIPSASGRAVAWAMYSIVQGLFGTGLWVLAHECGHSAFSDSNTVNNVTGWVLHSSMLVPYYAWKLTHSMHHKSTGHLTRDMVFVPKDRKEFMENRGAHDWSELAEDAPLMTLYGLITQQVFGWPLYLLSNVTGQKYPKLNKWAVNHFNPNAPLFEKKDWFNIWISNVGIGITMSVIAYSINRWGLASVTLYYLIPYLWVNHWLVAITYLQHTDPTLPHYHADQWNFTRGAAATIDREFGFIGSFCFHDIIETHVLHHYVSRIPFYNARIATEKIKKVMGKHYRHDDTNFIKSLYTVARTCQFVEGKEGIQMFRNVNGVGVAPDGLPSKK

SEQ ID NO: 3 - protein, 357 aa, Acheta domesticus
MDLNEESAPSGVLFEEDVAEQEAKMANGGPKKGKKLEEPYRLEIVWFNVLWFVLLHAGALYGVYLIFASAKIYTTLYGFLLCELSLLSITAGVHRLWAHRAYKAKWPLRLTLMVLNLLAYQNSIYEWARDHRVHHKFSETNADPVNAKRGFFFSHVGWLLCRKHPEVRAKGGRIDLSDLERDPIVMFQKRHYYKLVPFVSFVIPTLIPMYFWGETLSNSWYVSTMFRYCLSLNLTWLVNSAAHMWGNKPYDKNINPVENLAVAIGSLGEGWHNFHHVFPWDYKTSELGNYSLNFTNAFIDLAVLLGLAYDLKTVPVSMIKTRVGRTGDGSHDVWGWGDKDLPKELADQTMIENRKTE

SEQ ID NO: 4 - protein, 477 aa, Fusarium moniliforme
MASTSALPKQNPALRRTVTSTTVTDSESAAVSPSDSPRHSASSTSLSSMSEVDIAKPKSEYGVMLDTYGNQFEVPDFTIKDIYNAIPKHCFKRSALKGYGYILRDIVLLTTTFSIWYNFVTPEYIPSTPARAGLWAVYTVLQGLFGTGLWVIAHECGHGAFSDSRIINDITGWVLHSSLLVPYFSWQISHRKHHKATGNMERDMVFVPRTREQQATRLGKMTHELAHLTEETPAFTLLMLVLQQLVGWPNYLITNVTGHNYHERQREGRGKGKHNGLGGGVNHFDPRSPLYENSDAKLIVLSDIGIGLMATALYFLVQKFGFYNMAIWYFVPYLWVNHWLVAITFLQHTDPTLPHYTNDEWNFVRGAAATIDREMGFIGRHLLHGIIETHVLHHYVSSIPFYNADEATEAIKPIMGKHYRADVQDGPRGFIRAMYRSARMCQWVEPSAGAEGAGKGVLFFRNRNNVGTPPAVIKPVA

SEQ ID NO: 5 - protein, 456 aa, Ostreococcus tauri
MCVETENNDGIPTVEIAFDGERERAEANVKLSAEKMEPAALAKTFARRYVVIEGVEYDVTDFKHPGGTVIFYALSNTGADATEAFKEFHHRSRKARKALAALPSRPAKTAKVDDAEMLQDFAKWRKELERDGFFKPSPAHVAYRFAELAAMYALGTYLMYARYVVSSVLVYACFFGARCGWVQHEGGHSSLTGNIWWDKRIQAFTAGFGLAGSGDMWNSMHNKHHATPQKVRHDMDLDTTPAVAFFNTAVEDNRPRGFSKYWLRLQAWTFIPVTSGLVLLFWMFFLHPSKALKGGKYEELVWMLAAHVIRTWTIKAVTGFTAMQSYGLFLATSWVSGCYLFAHFSTSHTHLDVVPADEHLSWVRYAVDHTIDIDPSQGWVNWLMGYLNCQVIHHLFPSMPQFRQPEVSRRFVAFAKKWNLNYKVMTYAGAWKATLGNLDNVGKHYYVHGQHSGKTA

SEQ ID NO: 6 - protein, 457 aa, Mortierella alpina
MAAAPSVRTFTRAEILNAEALNEGKKDAEAPFLMIIDNKVYDVREFVPDHPGGSVILTHVGKDGTDVFDTFHPEAAWETLANFYVGDIDESDRAIKNDDFAAEVRKLRTLFQSLGYYDSSKAYYAFKVSFNLCIWGLSTFIVAKRGQTSTLANELSAALLGLFWQQRGWLAHDFLHHQVFQDRFWGDLFGAFLGGDCQGFSSSWWKDKHNTHHAAPNVHGEDPDIDTHPLLTWSEHALEMFSDVPDEELTRMWSRFMVLNQTWFYFPILSFARLSWCLQSILFVLPNGQAHKPSGARVPISLVEQLSLAMHWTWYLATMFLFIKDPVNMMVYFLVSQAVCGNLLAIVFSLNHNGMPVISKEEAVDMDFFTKQIITGRDVHPGLFANWFTGGLNYQIEHHLFPSMPRHNFSKIQPAVETLCKKYGVRYHTTGMIEGTAEVFSRLNEVSKAASKMGKAQ

SEQ ID NO: 7 - protein, 272 aa, Pavlova pinguis
MVAPPITLEWLLSPKLKDAVFGGEVLYFSIAYLFLAPILKRTPLVDTRKGAYKSGMIAYNVIMCVFSLVCFICQLAALGYDMGYLQWVRDLTGDEIVPLYQDVSPSPAFSNKLFKYSSIAFHYSKYVEYMDTAWLVMKGKPVSLLQGFHHFGAAWDTYFGITFQNEGIYVFVVLNAFIHTIMYAYYAATAAGLKFSLKFVITLMQITQFNVGFVMVYHYITLEYFRNSPELVFSYLFNYAYVCTVLLLFMQFFYMDNFGKKKAAAAAGKKKK

SEQ ID NO: 8 - protein, 279 aa, Pavlova salina
MATEGMPAITLDWLLSPGLKDAVIGGEVLYFSLGYLLLEPILKRSPFVDKRKGAYRNGMIAYNILMCGFSLVCFVCQMAALGLDRGHLQFVRDLTGDSVVQLYQDVSPSPAFANKLFRYSAVAFHYSKYVEYMDTAWLVLKGKPVSFLQGFHHFGAAWDTYFGITFQNEGTYVFVLLNAFIHTIMYTYYGATAAGIKISMKPLITLMQITQFLLGFALVYPYIDLGYFRASPELVWSYLFNYAYVLMVLFLFMRFFYHDNFSKHKPISRIDSSNRMKTE

SEQ ID NO: 9 - protein, 258 aa, Isochrysis galbana
MALANDAGERIWAAVTDPEILIGTFSYLLLKPLLRNSGLVDEKKGAYRTSMIWYNVLLALFSALSFYVTATALGWDYGTGAWLRRQTGDTPQPLFQCPSPVWDSKLFTWTAKAFYYSKYVEYLDTAWLRVSFLQAFHHFGAPWDVYLGIRLHNEGVWIFMFFNSFIHTIMYTYYGLTAAGYKFKAKPLITAMQICQFVGGFLLVWDYINVPCFNSDKGKLFSWAFNYAYVGSVFLLFCHFFYQDNLATKKSAKAGKQL

SEQ ID NO: 10 - protein, 263 aa, Isochrysis galbana
MALANDAGERIWAAVTDPEILIGTFSYLLLKPLLRNSGLVDEKKGAYRTSMIWYNVLLALFSALSFYVTATALGWDYGTGAWLRRQTGDTPQPLFQCPSPVWDSKLFTWTAKAFYYSKYVEYLDTAWLVLKGKRVSFLQAFHHFGAPWDVYLGIRLHNEGVWIFMFFNSFIHTIMYTYYGLTAAGYKFKAKPLITAMQICQFVGGFLLVWDYINVPCFNSDKGKLFSWAFNYAYVGSVFLLFCHFFYQDNLATKKSAKAGKQL

SEQ ID NO: 11 - protein, 261 aa, Isochrysis galbana
MATEATASIWAAVSDPEILIGTFSYLLLKPILRSSGLVDEKKGAYRTSMIWYNVILALFSATSFYVTATALGWDYGSGEWLRRLTGDTPQPLFQCPSRVWDSKLFVWTAKAFYYSKYVEYLDTAWLVLKGKNVSFLQAFHHFGAPWDVYLGIRLQNEGVWIFMFFNSFIHTIMYTYYGLTAAGYKIKAKPLITAMQISQFMGGFILVWDYINIPCFRSDNGKVFSWVFNYAYVGFVFLLFCHFFYKDNLASKKPAKGGKAL

SEQ ID NO: 12 - protein, 266 aa, Emiliania huxleyi
MLDRASSDAAIWSAVSDPEILIGTFSYLLLKPLLRNSGLVDERKGAYRTSMIWYNVVLALFSATSFYVTATALGWDKGTGEWLRSLTGDSPQQLWQCPSRVWDSKLFLWTAKAFYYSKYVEYLDTAWLVLKGKKVSFLQGFHHFGAPWDVYLGIRLKNEGVWIFMFFNSFIHTVMYTYYGLTAAGYKIRGKPIITAMQISQFVGGFVLVWDYINVPCFHADAGQVFSWVFNYAYVGSVFLLFCHFFYMDNIAKAKAKKAVATRKAL

SEQ ID NO: 13 - protein, 297 aa, Pyramimonas cordata
MEFAQPLVAMAQEQYAAIDAVVAPAIFSATDSIGWGLKPISSATKDLPLVESPTPLILSLLAYFAIVGSGLVYRKVFPRTVKGQDPFLLKALMLAHNVFLIGLSLYMCLKLVYEAYVNKYSFWGNAYNPAQTEMAKVIWIFYVSKIYEFMDTFIMLLKGNVNQVSFLHVYHHGSISGIWWMITYAAPGGDAYFSAALNSWVHVCMYTYYFMAAVLPKDEKTKRKYLWWGRYLTQMQMFQFFMNLLQAVYLLYSSSPYPKFIAQLLVVYMVTLLMLFGNFYYMKHHASKKQKLASKKQ

SEQ ID NO: 14 - protein, 427 aa, Pavlova salina
MGRGGDSSGQAHPAAELAVPSDRAEVSNADSKALHIVLYGKRVDVTKFQRTHPGGSKVFRIFQDRDATEQFESYHSKRAIKMMEGMLKKSEDAPADTPLPSQSPMGKDFKAMIERHVAAGYYDPCPLDELFKLSLVLLPTFAGMYMLKAGVGSPLCGALMVSFGWYLDGWLAHDYLHHSVFKGSVARTVGWNNAAGYFLGFVQGYAVEWWRARHNTHHVCTNEDGSDPDIKTAPLLIYVRNKPSIAKRLNAFQRYQQYYYVPVMAILDLYWRLESIAYVAMRLPKMLPQALALVAHYAIVAWVFAGNYHLLPLVTVLRGFGTGITVFATHYGEDILDADQVRHMTLVEQTALTSRNISGGWLVNVLTGFISLQTEHHLFPMMPTGNLMTIQPEVRAFFKKHGLEYREGNLIECVRQNIRALAFEHLL

SEQ ID NO: 15 - protein, 425 aa, Pavlova salina
MPPRDSYSYAAPPSAQLHEVDTPQEHDKKELVIGDRAYDVTNFVKRHPGGKIIAYQVGTDATDAYKQFHVRSAKADKMLKSLPSRPVHKGYSPRRADLIADFQEFTKQLEAEGMFEPSLPHVAYRLAEVIAMHVAGAALIWHGYTFAGIAMLGVVQGRCGWLMHEGGHYSLTGNIAFDRAIQVACYGLGCGMSGAWWRNQHNKHHATPQKLQHDVDLDTLPLVAFHERIAAKVKSPAMKAWLSMQAKLFAPVTTLLVALGWQLYLHPRHMLRTKHYDELAMLGIRYGLVGYLAANYGAGYVLACYLLYVQLGAMYIFCNFAVSHTHLPVVEPNEHATWVEYAANHTTNCSPSWWCDWWMSYLNYQIEHHLYPSMPQFRHPKIAPRVKQLFEKHGLHYDVRGYFEAMADTFANLDNVAHAPEKKMQ

SEQ ID NO: 16 - protein, 446 aa, Mortierella alpina
MGTDQGKTFTWEELAAHNTKDDLLLAIRGRVYDVTKFLSRHPGGVDTLLLGAGRDVTPVFEMYHAFGAADAIMKKYYVGTLVSNELPIFPEPTVFHKTIKTRVEGYFTDRNIDPKNRPEIWGRYALIFGSLIASYYAQLFVPFVVERTWLQVVFAIIMGFACAQVGLNPLHDASHFSVTHNPTVWKILGATHDFFNGASYLVWMYQHMLGHHPYTNIAGADPDVSTSEPDVRRIKPNQKWFVNHINQHMFVPFLYGLLAFKVRIQDINILYFVKTNDAIRVNPISTWHTVMFWGGKAFFVWYRLIVPLQYLPLGKVLLLFTVADMVSSYWLALTFQANHVVEEVQWPLPDENGIIQKDWAAMQVETTQDYAHDSHLWTSITGSLNYQAVHHLFPNVSQHHYPDILAIIKNTCSEYKVPYLVKDTFWQAFASHLEHLRVLGLRPKEE

SEQ ID NO: 17 - protein, 267 aa, Pyramimonas cordata
MASIAIPAALAGTLGYVTYNVANPDIPASEKVPAYFMQVEYWGPTIGTIGYLLFIYFGKRIMQNRSQPFGLKNAMLVYNFYQTFFNSYCIYLFVTSHRAQGLKVWGNIPDMTANSWGISQVIWLHYNNKYVELLDTFFMVMRKKFDQLSFLHIYHHTLLIWSWFVVMKLEPVGDCYFGSSVNTFVHVIMYSYYGLAALGVNCFWKKYITQIQMLQFCICASHSIYTAYVQNTAFWLPYLQLWVMVNMFVLFANFYRKRYKSKGAKKQ

SEQ ID NO: 18 - protein, 447 aa, Pavlova salina
MPPSAAKQMGASTGVHAGVTDSSAFTRKDVADRPDLTIVGDSVYDAKAFRSEHPGGAHFVSLFGGRDATEAFMEYHRRAWPKSRMSRFHVGSLASTEEPVAADEGYLQLCARIAKMVPSVSSGFAPASYWVKAGLILGSAIALEAYMLYAGKRLLPSIVLGWLFALIGLNIQHDANHGALSKSASVNLALGLCQDWIGGSMILWLQEHVVMHHLHTNDVDKDPDQKAHGALRLKPTDAWSPMHWLQHLYLLPGETMYAFKLLFLDISELVMWRWEGEPISKLAGYLFMPSLLLKLTFWARFVALPLYLAPSVHTAVCIAATVMTGSFYLAFFFFISHNFEGVASVGPDGSITSMTRGASFLKRQAETSSNVGGPLLATLNGGLNYQIEHHLFPRVHHGFYPRLAPLVKAELEARGIEYKHYPTIWSNLASTLRHMYALGRRPRSKAE

SEQ ID NO: 19 - protein, 519 aa, Thraustochytrium
MTVGYDEEIPFEQVRAHNKPDDAWCAIHGHVYDVTKFASVHPGGDIILLAAGKEATVLYETYHVRGVSDAVLRKYRIGKLPDGQGGANEKEKRTLSGLSSASYYTWNSDFYRVMRERVVARLKERGKARRGGYELWIKAFLLLVGFWSSLYWMCTLDPSFGAILAAMSLGVFAAFVGTCIQHDGNHGAFAQSRWVNKVAGWTLDMIGASGMTWEFQHVLGHHPYTNLIEEENGLQKVSGKKMDTKLADQESDPDVFSTYPMMRLHPWHQKRWYHRFQHIYGPFIFGFMTINKVVTQDVGVVLRKRLFQIDAECRYASPMYVARFWIMKALTVLYMVALPCYMQGPWHGLKLFAIAHFTCGEVLATMFIVNHIIEGVSYASKDAVKGTMAPPKTMHGVTPMNNTRKEVEAEASKSGAVVKSVPLDDWAAVQCQTSVNWSVGSWFWNHFSGGLNHQIEHHLFPGLSHETYYHIQDVVQSTCAEYGVPYQHEPSLWTAYWKMLEHLRQLGNEETHESWQRAA

SEQ ID NO: 20 - DNA, 33 bp, Artificial Sequence
  Synthetic primer
gggggcgcctagggataacagggtaatgataac

SEQ ID NO: 21 - DNA, 32 bp, Artificial Sequence
  Synthetic primer
cccggcgccattaccctgttatccctactcgc

SEQ ID NO: 22 - DNA, 34 bp, Artificial Sequence
  Synthetic primer
gggggcgcgccgagtcgactttccgatacagaag

SEQ ID NO: 23 - DNA, 34 bp, Artificial Sequence
  Synthetic primer
cccgcgatcgctgatggttgtgatcaaccggaaa

SEQ ID NO: 24 - DNA, 36 bp, Artificial Sequence
  Synthetic primer
ggggcgatcgcagagtccgttttgtagagtaatatg

SEQ ID NO: 25 - DNA, 35 bp, Artificial Sequence
  Synthetic primer
cccgcggccgcacggacctcgcaagttgcaaaaag

SEQ ID NO: 26 - DNA, 31 bp, Artificial Sequence
  Synthetic primer
gggggcgcgccatgctgcgggcggatcctgg

SEQ ID NO: 27 - DNA, 35 bp, Artificial Sequence
  Synthetic primer
cccgcgatcgcagcttttgttttgtgtgacttgtc

SEQ ID NO: 28 - DNA, 32 bp, Artificial Sequence
  Synthetic primer
ggggcgatcgcggaaaactgcctgggttaggc

SEQ ID NO: 29 - DNA, 32 bp, Artificial Sequence
  Synthetic primer
cccgcggccgctctgatggcctggagcgagtt

SEQ ID NO: 30 - DNA, 35 bp, Artificial Sequence
  Synthetic primer
gggggcgcgcctgggagtgtatttggaaaatgact

SEQ ID NO: 31 - DNA, 31 bp, Artificial Sequence
  Synthetic primer
cccgcgatcgctttgcgggcggtacgggtac

SEQ ID NO: 32 - DNA, 36 bp, Artificial Sequence
  Synthetic primer
ggggcgatcgccataacactcatcagtagcctttac

SEQ ID NO: 33 - DNA, 39 bp, Artificial Sequence
  Synthetic primer
cccgcggccgctcttgtaattccatagataatatatacg

SEQ ID NO: 34 - DNA, 34 bp, Artificial Sequence
  Synthetic primer
gggggcgcgccgccgacaatctctcttcctcatt

SEQ ID NO: 35 - DNA, 33 bp, Artificial Sequence
  Synthetic primer
cccgcgatcgctgtgtgtgcggagagtgtcttg

SEQ ID NO: 36 - DNA, 32 bp, Artificial Sequence
  Synthetic primer
ggggcgatcgcgcacagtcgcttcaccacttg

SEQ ID NO: 37 - DNA, 37 bp, Artificial Sequence
  Synthetic primer
cccgcggccgctaaaacaatgacaaatacaactctag

SEQ ID NO: 38 - DNA, 35 bp, Artificial Sequence
  Synthetic primer
ggggcgatcgctagggataacagggtaatgataac

SEQ ID NO: 39 - DNA, 34 bp, Artificial Sequence
  Synthetic primer
cccgcgatcgcattaccctgttatccctactcgc

SEQ ID NO: 40 - DNA, 19 bp, Artificial Sequence
  Synthetic primer
gtgtccgagcccgtctacg

SEQ ID NO: 41 - DNA, 22 bp, Artificial Sequence
  Synthetic primer
acatggtgataccaataccgac

SEQ ID NO: 42 - DNA, 20 bp, Artificial Sequence
  Synthetic primer
gatgcaaatgacgcacggcc

SEQ ID NO: 43 - DNA, 20 bp, Artificial Sequence
  Synthetic primer
ccaaacgacactcgatagtg

SEQ ID NO: 44 - DNA, 20 bp, Artificial Sequence
  Synthetic primer
tgctttgcgtgggagagctc

SEQ ID NO: 45 - DNA, 24 bp, Artificial Sequence
  Synthetic primer
cagtggttgacattctataggcac

SEQ ID NO: 46 - DNA, 21 bp, Artificial Sequence
  Synthetic primer
caggcaacagacaagtcacac

SEQ ID NO: 47 - DNA, 20 bp, Artificial Sequence
  Synthetic primer
catctggctccaacggccac

SEQ ID NO: 48 - DNA, 20 bp, Artificial Sequence
  Synthetic primer
cagctgtgggtcgttttcgc

SEQ ID NO: 49 - DNA, 20 bp, Artificial Sequence
  Synthetic primer
gctatttgcctaacccaggc

SEQ ID NO: 50 - DNA, 21 bp, Artificial Sequence
  Synthetic primer
catagctacacccaaagtcgc

SEQ ID NO: 51 - DNA, 20 bp, Artificial Sequence
  Synthetic primer
cgctcagcggactgaccatg

SEQ ID NO: 52 - DNA, 20 bp, Artificial Sequence
  Synthetic primer
tcttggggtacacgggctgg

SEQ ID NO: 53 - DNA, 22 bp, Artificial Sequence
  Synthetic primer
ctgtaaaggctactgatgagtg

SEQ ID NO: 54 - DNA, 22 bp, Artificial Sequence
  Synthetic primer
catgactaatcattcacgcgac

SEQ ID NO: 55 - DNA, 20 bp, Artificial Sequence
  Synthetic primer
cgtggtcggtgtgatagccg

SEQ ID NO: 56 - DNA, 20 bp, Artificial Sequence
  Synthetic primer
gttcgcagcagatcggctcg

SEQ ID NO: 57 - DNA, 22 bp, Artificial Sequence
  Synthetic primer
cgaacgcccttctccatcagtg

SEQ ID NO: 58 - DNA, 20 bp, Artificial Sequence
  Synthetic primer
cgttggaggctgtgggtctg

SEQ ID NO: 59 - DNA, 20 bp, Artificial Sequence
  Synthetic primer
agtgctcaagctcgtggcag

SEQ ID NO: 60 - DNA, 20 bp, Artificial Sequence
  Synthetic primer
gatctcggttctggccgtac

SEQ ID NO: 61 - DNA, 20 bp, Artificial Sequence
  Synthetic primer
gcctccaggaagtccatggg

SEQ ID NO: 62 - DNA, 3260 bp, Yarrowia lipolytica
aactatacaagtatcaagttgcccattgtgttgtatgttccagaaccattgtacccgtacaagtagttaagcatagcccttgtgcaagagtgtgtacgatcaatgtctcagatacggcggtgactgccgcttctggcagtctttcaacgctgcgtttcttcgtcgatggaccggaagagctctctttacgagccaaaggctccaagagtttggttgtcttgtactctctatctttccacaccgccgcccacaaggttcggaacgacatctcccaactctaactttaagcatggcctgttgagaagccatgctattcatcggacacctgttcaacgagtcaacaagcggacatagtcgtcaagtagtggcagcacagagagagagtcgtgccaatgggggtcatgtagtagcacatgacgaaataagagacgttggattcatgtagaaccgacgtagtcacgtgaacacgtccaaggtcttcccctcttcacgtgcacgtgtaactggttctttcactctcccccccgccataagtcgacccatgtcacggttgagacgtggtccctccacactatccacgctgagagcagcagtataccccgggcggttccgttcggagctctcttggccaatccgcacatgcgcattatgcgtttttcaggtggtaacagacgaggagtacgagcgtgttaaagtggcggttaggagaggcggggcccgtagttatatacaagaggtagatgcgtgctggtgttagaggggctctcaggattaggaggaaaatttgacattggccctcaacatataacctcgggtgtgcctctgtttaccctcagcttttgcttgtccccaagtcagtcacgccaggccaaaaaggttggtggattgacagggagaaaaaaaaaagcctagtgggtttaaactcgaggtaagacattgaaatatataccggtcggcatcctgagtccctttctcgtattccaacagaccgaccatagaaatggattcgaccacgcagaccaacaccggcaccggcaaggtggccgtgcagccccccacggccttcattaagcccattgagaaggtgtccgagcccgtctacgacacctttggcaacgagttcactcctccagactactctatcaaggatattctggatgccattccccaggagtgctacaagcggtcctacgttaagtcctactcgtacgtggcccgagactgcttctttatcgccgtttttgcctacatggcctacgcgtacctgcctcttattccctcggcttccggccgagctgtggcctgggccatgtactccattgtccagggtctgtttggcaccggtctgtgggttcttgcccacgagtgtggccactctgctttctccgactctaacaccgtcaacaacgtcaccggatgggttctgcactcctccatgctggtcccttactacgcctggaagctgacccactccatgcaccacaagtccactggtcacctcacccgtgatatggtgtttgtgcccaaggaccgaaaggagtttatggagaaccgaggcgcccatgactggtctgagcttgctgaggacgctcccctcatgaccctctacggcctcatcacccagcaggtgtttggatggcctctgtatctgctgtctaacgttaccggacagaagtaccccaagctcaacaaatgggctgtcaaccacttcaaccccaacgccccgctgtttgagaagaaggactggttcaacatctggatctctaacgtcggtattggtatcaccatgtccgtcatcgcatactccatcaaccgatggggcctggcttccgtcaccctctactacctgatcccctacctgtgggtcaaccactggctcgtggccatcacctacctgcagcacaccgaccccactctgccccactaccacgccgaccagtggaacttcacccgaggagccgccgccaccatcgaccgagagtttggcttcatcggctccttctgcttccatgacatcatcgagacccacgttctgcaccactacgtgtctcgaattcccttctacaacgcccgaatcgccactgagaagatcaagaaggtcatgggcaagcactaccgacacgacgacaccaacttcatcaagtctctttacactgtcgcccgaacctgccagtttgttgaaggtaaggaaggcattcagatgtttagaaacgtcaatggagtcggagttgctcctgacggcctgccttctaaaaagtagagctagaaatgttatttgattgtgttttaactgaacagcaccgagcccgaggctaagccaagcgaagccgaggggttgtgtagtccatggacgtaacgagtaggcgatatcaccgcactcggcactgcgtgtctgcgttcatgggcgaagtcacattacgctgacaaccgttgtagtttccctttagtatcaatactgttacaagtaccggtactcgtactcgtactgatacgaatctgtgggaagaagtcacccttatcagaccttcatactgatgtttcggatatcaatagaactggcatagagccgttaaagaagtttcacttaatcactccaaccctcctacttgtagattcaagcagatcgataagatggatttgatggtcagtgctagcctgggcccgagcgtcaccatcagtcattctttacaagttgttccactcttctccctacttgaatcgatctgtctgggcgcctgatattcgttcgacttgtacgatagcggggtgtatttcgagctcgccgaaaccggttttccatgaatttccaccgataattccattggcaattcaatcgaccataagcgtatccggtaacggtgagacattgagacaccgatcgattagagagactactgtacgagcagtctactgtatattagaaggaacaataggtagtccaactgagcttttaactagtgggtgttttttgaacgtgtttggagatcttattaagcaaaaaaaaagctgtgttcacattaacaccaacaaaccctgccggtagttccttcaaaaacgccacatgcatcaatcatcttcagctttgccgatctcctcctgccgaggttacaaccaactggagccatctttatcgtgccgtagagaagcgcagccagggagagtttgagaccggcaaaacgagggaatcggtagattctcagaaataatccatatggagtcacgtgatcagacttcccatggacttttatattgatatttgt

SEQ ID NO: 63 - DNA, 1620 bp, Artificial Sequence
  Synthetic resistance marker cassette
tagggataacagggtaatgataacttcgtatagcatacattatacgaagttatgggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagcacaatgaagaagcccgagctgaccgctacctctgttgagaagttcctgattgagaagtttgattccgtttccgacctgatgcagctgtccgagggcgaggagtctcgagccttctcctttgacgtgggcggacgaggttacgttctgcgagtgaactcgtgtgccgacggcttctacaaggatcgatacgtctaccgacactttgcttctgccgctctgcccatccctgaggttctcgacattggcgagttctctgagtccctcacctactgcatctctcgacgagctcagggagtcaccctgcaggacctccctgagactgagctgcctgctgtcctccagcctgttgctgaggccatggacgctatcgctgctgctgatctgtcccagacctcgggtttcggcccctttggacctcagggaattggacagtacaccacttggcgagacttcatctgtgctattgccgatcctcacgtctaccattggcagaccgttatggacgatactgtgtcggcttctgtcgctcaggctctggacgagctgatgctctgggccgaggattgccccgaggttcgacacctggtgcatgctgacttcggttccaacaacgttctcaccgacaacggccgaatcactgccgtgattgactggtccgaggctatgtttggcgactcgcagtacgaggtggccaacatcttcttttggcgaccctggctggcttgtatggagcagcagacccgatacttcgagcgacgacatcctgagctcgctggctcccctcgactgcgagcttacatgctccgaattggtctggaccagctctaccagtcgctggtggatggcaactttgacgatgctgcctgggctcagggacgatgtgacgccatcgtgcgatctggcgctggaaccgtcggacgaactcagattgcccgacgatccgctgctgtctggaccgacggatgcgtggaggtcctggctgattcgggtaaccgacgaccctctactcgacctcgagctaaggagtaagtgtctgtggtatctaagctatttatcactctttacaacttctacctcaactatctactttaataaatgaatatcgtttattctctatgattactgtatatgcgttcctctaagacaaatcgaatataacttcgtatagcaggagttatccgaagcgagtagggataacagggtaat

SEQ ID NO: 64 - protein, 341 aa, Artificial Sequence
  Synthetic resistance marker
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

SEQ ID NO: 65 - DNA, 1165 bp, Artificial Sequence
  Synthetic resistance marker cassette
tagggataacagggtaatgataacttcgtatagcatacattatacgaagttatggggttggcggcgcatttgtgtcccaaaaaacagccccaattgccccaattgaccccaaattgacccagtagcggacccaaccccggcgagagcccccttcaccccacatatcaaacctcccccggttcccacacttgccgttaagggcgtagggtactgcagtctggaatctacgcttgttcagactttgtactagtttctttgtctggccatccgggtaacccatgccggacgcaaaatagactactgaaaatttttttgctttgtggttgggactttagccaagggtataaaagaccaccgtccccgaattacctttcctcttcttttctctctctccttgtcaactcacacccgaagcacaatgaccactctggatgacaccgcttaccgataccgaacttccgttcctggcgatgccgaggctattgaggctctggatggatctttcaccactgacaccgttttccgagtgaccgctactggcgacggcttcaccctgcgagaggtgcctgtcgaccctcctctcaccaaggttttccctgacgatgagtcggacgatgagtctgacgctggagaggacggcgaccctgactctcgaactttcgtggcttacggcgacgatggcgacctggccggctttgtggtcgtttcttactccggatggaaccgacgactgaccgtggaggacatcgaggtcgctcctgagcaccgaggtcatggtgtcggacgagctctgatgggtctggctactgagttcgctcgagagcgaggtgctggccacctgtggctcgaggtcaccaacgttaacgcccctgctattcatgcctaccgacgaatgggttttaccctgtgtggcctcgatactgccctgtacgacggaaccgcttccgatggagagcaggccctctacatgtcgatgccctgcccttaagtgtctgtggtatctaagctatttatcactctttacaacttctacctcaactatctactttaataaatgaatatcgtttattctctatgattactgtatatgcgttcctctaagacaaatcgaatataacttcgtatagcaggagttatccgaagcgagtagggataacagggtaat

SEQ ID NO: 66 - protein, 189 aa, Artificial Sequence
  Synthetic resistance marker
MTTLDDTAYRYRTSVPGDAEAIEALDGSFTTDTVFRVTATGDGFTLREVPVDPPLTKVFPDDESDDESDAGEDGDPDSRTFVAYGDDGDLAGFVVVSYSGWNRRLTVEDIEVAPEHRGHGVGRALMGLATEFARERGAGHLWLEVTNVNAPAIHAYRRMGFTLCGLDTALYDGTASDGEQALYMSMPCP

SEQ ID NO: 67 - protein, 286 aa, Yarrowia lipolytica
MPSYEARANVHKSAFAARVLKLVAAKKTNLCASLDVTTTKELIELADKVGPYVCMIKTHIDIIDDFTYAGTVLPLKELALKHGFFLFEDRKFADIGNTVKHQYKNGVYRIAEWSDITNAHGVPGTGIIAGLRAGAEETVSEQKKEDVSDYENSQYKEFLVPSPNEKLARGLLMLAELSCKGSLATGEYSKQTIELARSDPEFVVGFIAQNRPKGDSEDWLILTPGVGLDDKGDALGQQYRTVEDVMSTGTDIIIVGRGLYGQNRDPIEEAKRYQKAGWEAYQKINC

SEQ ID NO: 68 - DNA, 2861 bp, Yarrowia lipolytica
ttaccattttaaagcaattgttaacaagatactactgtagaaaactgattaaacattcgtaacttgcctgtggctcgacttacagccaaatgttcgcggcaaaatttgaatattttgcacacttattgtcccaaaaccaaataatgccaacaaaatccgatatagtgtaggggctatcacataccctctcataagaatacttttaaccgtggagacggggttcgattcgtatcggagtgtttattttttgctcaaccataccctggggtgtgttctgtggagcattctcacttttggtaaacgacattgcttcaagtgcagcggaatcaaaaagtataaagtgggcagcgagtatacctgtacagactgtaggcgataactcaatccaattaccccccacaacatgactggccaaactgatctcaagactttattgaaatcagcaacaccgattctcaatgaaggcacatacttcttctgcaacattcacttgacgcctaaagttggtgagaaatggaccgacaagacatattctgctatccacggactgttgcctgtgtcggtggctacaatacgtgagtcagaagggctgacggtggtggttcccaaggaaaaggtcgacgagtatctgtctgactcgtcattgccgcctttggagtacgactccaactatgagtgtgcttggatcactttgacgatacattcttcgttggaggctgtgggtctgacagctgcgttttcggcgcggttggccgacaacaatatcagctgcaacgtcattgctggctttcatcatgatcacatttttgtcggcaaaggcgacgcccagagagccattgacgttctttctaatttggaccgatagccgtatagtccagtctatctataagttcaactaactcgtaactattaccataacatatacttcactgccccagataaggttccgataaaaagttctgcagactaaatttatttcagtctcctcttcaccaccaaaatgccctcctacgaagctcgagctaacgtccacaagtccgcctttgccgctcgagtgctcaagctcgtggcagccaagaaaaccaacctgtgtgcttctctggatgttaccaccaccaaggagctcattgagcttgccgataaggtcggaccttatgtgtgcatgatcaagacccatatcgacatcattgacgacttcacctacgccggcactgtgctccccctcaaggaacttgctcttaagcacggtttcttcctgttcgaggacagaaagttcgcagatattggcaacactgtcaagcaccagtacaagaacggtgtctaccgaatcgccgagtggtccgatatcaccaacgcccacggtgtacccggaaccggaatcattgctggcctgcgagctggtgccgaggaaactgtctctgaacagaagaaggaggacgtctctgactacgagaactcccagtacaaggagttcctggtcccctctcccaacgagaagctggccagaggtctgctcatgctggccgagctgtcttgcaagggctctctggccactggcgagtactccaagcagaccattgagcttgcccgatccgaccccgagtttgtggttggcttcattgcccagaaccgacctaagggcgactctgaggactggcttattctgacccccggggtgggtcttgacgacaagggagacgctctcggacagcagtaccgaactgttgaggatgtcatgtctaccggaacggatatcataattgtcggccgaggtctgtacggccagaaccgagatcctattgaggaggccaagcgataccagaaggctggctgggaggcttaccagaagattaactgttagaggttagactatggatatgtaatttaactgtgtatatagagagcgtgcaagtatggagcgcttgttcagcttgtatgatggtcagacgacctgtctgatcgagtatgtatgatactgcacaacctgtgtatccgcatgatctgtccaatggggcatgttgttgtgtttctcgatacggagatgctgggtacaagtagctaatacgattgaactacttatacttatatgaggcttgaagaaagctgacttgtgtatgacttattctcaactacatccccagtcacaataccaccactgcactaccactacaccaaaaccatgatcaaaccacccatggacttcctggaggcagaagaacttgttatggaaaagctcaagagagagaagccaagatactatcaagacatgtgtcgcaacttcaaggaggaccaagctctgtacaccgagaaacaggcctttgtcgacgaagctctaaagctgaaacgggacatcgaaaagttggagaagtcaatctggaaactacacgtagaaaacagacaccatctccaacaagtacaggacttcaaatttgcactgaagacattctgtgggtggagccataagtaacaatcgcctgctccaggaacttaaaaaagaggcattataggccccttcatctgaatattctttgagtaccaactgccgccaactccccaatcgttaattagtgttatctattctatctatgctattagcacatatttatcatgtgctgctgttatgtacgggttcaatatacaaatgcttagtcctcaaacaccttcataggtgggtcgtcttcctggccaaatcgaattctcatgatttcaaacacgaacagaccgatagacgtagacggaagcgcactgagggtcgtccagccacaaccatggtataggtatctgggccaactcaaccgcagaccctttcggatgttattggctgtatgcagagtctcaacgtaggctcggtagtagagatg

SEQ ID NO: 69 - DNA, 3545 bp, Yarrowia lipolytica
atgctgcgggcggatcctggtgcatttttgcttgcgattgttgtgaaaattagcacggttatattagcccgtaataaatgcccgtctccatcttcatatggccatcaccccgcaaatagccggccaatcaggcgtatgtcacctgttgctcacacgcatgtcctcggaccgttgtattgtgcaagtaggggtacctccccgatccatcctcgaccagtggcacgctcaaccccatggttcgcttttctcttttcgtctatcctgaactgagtttttttccacgccaactgatatccccttacgttaccccctcatcacctggtgaggcgaaactgtaaggtgaaagctaaaaatgacatctcagctgcacgaaggaccggggcttaaaagacgggctggtgcttgtgatttaaaactggacaaatctcagcttgcttgaaattttggtctccaactgtttccgagcgaatcgcacacaaaccgggcttctctctgcagaccacgcccccgaaactctttctcccaccaccaccaacactccctttccattcccacaccgttcctctctcgtgcgtcatccttgcgcaatcatcttcgtctgcgacatattgtacgacatacagtaccacggaacgtttcagaccgtcgacgtgaacacatcttaggaacagcaacctgagctacagaaatctatctataggcggataaaaaaacgcacccactgctcgtcctccttgctcctcgaaaccgactcctctacacacgtcaaatccgaggttgaaatcttccccacatttggcagccaaaccagcacatcccagcaacctcgcacagcgccgaaatcgacctgtcgacttggccacaaaaaaaagcaccggctctgcaacagttctcacgaccaattacgtacaagtacgaaatcgttcgtggaccgtgactgataagctcccactttttcttctaacaacaggcaacagacaagtcacacaaaacaaaagctatgactatcgactcacaatactacaagtcgcgagacaaaaacgacacggcacccaaaatcgcgggaatccgatatgccccgctatcgacaccattactcaaccgatgtgagaccttctctctggtctggcacattttcagcattcccactttcctcacaattttcatgctatgctgcgcaattccactgctctggccatttgtgattgcgtatgtagtgtacgctgttaaagacgactccccgtccaacggaggagtggtcaagcgatactcgcctatttcaagaaacttcttcatctggaagctctttggccgctacttccccataactctgcacaagacggtggatctggagcccacgcacacatactaccctctggacgtccaggagtatcacctgattgctgagagatactggccgcagaacaagtacctccgagcaatcatctccaccatcgagtactttctgcccgccttcatgaaacggtctctttctatcaacgagcaggagcagcctgccgagcgagatcctctcctgtctcccgtttctcccagctctccgggttctcaacctgacaagtggattaaccacgacagcagatatagccgtggagaatcatctggctccaacggccacgcctcgggctccgaacttaacggcaacggcaacaatggcaccactaaccgacgacctttgtcgtccgcctctgctggctccactgcatctgattccacgcttcttaacgggtccctcaactcctacgccaaccagatcattggcgaaaacgacccacagctgtcgcccacaaaactcaagcccactggcagaaaatacatcttcggctaccacccccacggcattatcggcatgggagcctttggtggaattgccaccgagggagctggatggtccaagctctttccgggcatccctgtttctcttatgactctcaccaacaacttccgagtgcctctctacagagagtacctcatgagtctgggagtcgcttctgtctccaagaagtcctgcaaggccctcctcaagcgaaaccagtctatctgcattgtcgttggtggagcacaggaaagtcttctggccagacccggtgtcatggacctggtgctactcaagcgaaagggttttgttcgacttggtatggaggtcggaaatgtcgcccttgttcccatcatggcctttggtgagaacgacctctatgaccaggttagcaacgacaagtcgtccaagctgtaccgattccagcagtttgtcaagaacttccttggattcacccttcctttgatgcatgcccgaggcgtcttcaactacgatgtcggtcttgtcccctacaggcgacccgtcaacattgtggttggttcccccattgacttgccttatctcccacaccccaccgacgaagaagtgtccgaataccacgaccgatacatcgccgagctgcagcgaatctacaacgagcacaaggatgaatatttcatcgattggaccgaggagggcaaaggagccccagagttccgaatgattgagtaaggaaaactgcctgggttaggcaaatagctaatgagtatttttttgatggcaaccaaatgtagaaagaaaaaaaaaaaaaaagaaaaaaaaaagagaatattatatctatgtaattctattaaaagctctgttgagtgagcggaataaatactgttgaagaggggattgtgtagagatctgtttactcaatggcaaactcatctgggggagatccttccactgtgggaagctcctggatagcctttgcatcgggttcaagaagaccattgtgaacagccttgacactgtcgacaatgacagcatcaacgccatgcttgatctgggtcttggcaatcacagggttgttattcagaacaccgtaggagacacagaccagcccagcccgcttgacgacctggataagtcgagggcacatgacaaagggactacactcggacacaagacccaaaaggttccaatccttgcagaaccgaatggcctcctggagagaagaggctcgaatgtcatacatgggggcagttccagactcggtgaggaacagggtggggtatccgacagggttgacttcagtcttgccaatcatagggacacctccttgcttgtggttcagcatgaggcacacgtcagggtggaaagaggagaagataatctctcggtttgctccatactcaaagacacactcgaggaccttgtccgcccaaacgttgaggtcaagggcaaactcttccatctcctcagcttgtccttcgtcaagcatggggtacttgcactcgatgttgcagccaacgttgggagggagagactgtagaacctctcgtagagtggtgaaaggggcctgaatggaaataccacgagaattacccttgaaacccttcatcttgtagtcacgagtgtacttcattcgatgagtgatgtggtcatctgcgtcagggtcatcttgctgaagtgcagttccttcaagtatcttgtcgccctcagaaactcgctccaggccatcaga

SEQ ID NO: 70 - protein, 514 aa, Yarrowia lipolytica
MTIDSQYYKSRDKNDTAPKIAGIRYAPLSTPLLNRCETFSLVWHIFSIPTFLTIFMLCCAIPLLWPFVIAYVVYAVKDDSPSNGGVVKRYSPISRNFFIWKLFGRYFPITLHKTVDLEPTHTYYPLDVQEYHLIAERYWPQNKYLRAIISTIEYFLPAFMKRSLSINEQEQPAERDPLLSPVSPSSPGSQPDKWINHDSRYSRGESSGSNGHASGSELNGNGNNGTTNRRPLSSASAGSTASDSTLLNGSLNSYANQIIGENDPQLSPTKLKPTGRKYIFGYHPHGIIGMGAFGGIATEGAGWSKLFPGIPVSLMTLTNNFRVPLYREYLMSLGVASVSKKSCKALLKRNQSICIVVGGAQESLLARPGVMDLVLLKRKGFVRLGMEVGNVALVPIMAFGENDLYDQVSNDKSSKLYRFQQFVKNFLGFTLPLMHARGVFNYDVGLVPYRRPVNIVVGSPIDLPYLPHPTDEEVSEYHDRYIAELQRIYNEHKDEYFIDWTEEGKGAPEFRMIE

SEQ ID NO: 71 - DNA, 3581 bp, Yarrowia lipolytica
tgggagtgtatttggaaaatgacttggcctggaccccgactaccgctagtgctacccatggggtgtctccagaacccttgtacatcagcctatatgcgtgtgtgtctacgtctactgtacaaatctgcttggcgtcaaatggcactacaagtacaaagaccaaatgccacaagttcagtacgagtacaagagtctgctacttgtagctcccgcaggtatttagttcatacaaacgagtatacttgtagctgtccaacagtagttttcagacacctgatcagaaataggaacacgctccacggacctcgtatccactctaagaccataatatatgggtttctggaacattggtgcccctcacctctcatctcttttttttttacctctctgaccaccaccacccttgttgttccgagtggttagctgcaccccacctagcaaactttagggccccatacgcataagcatacgtccggtgtgcgacttgtgtgtgaacgcggtccacacaaggtgtctttcaagtccacacagggctttgtctagagcctgggctatcttcccacgtttgtatatatagactggatccacacccagcaacgcaacgtcctcccacccaaccctatcctccacgttacacgcccaagatcttccaaacaagaagacacattccacagcaagaataaccttggcaagaacgtgcgacgaggtgaataaaagagcccagagtactaacattttgtggacctgttggaaactaattgccgccgcaactcgactggaaccaagttcaagaccactgacaagcgccccggtccaaccagaacactaaacggagccgcacagcaagcaccacggacgatcgtcatcatccctatggccaacctgcatggatactcttgataacagttaagctatatttaccaacaccacctccgttcacgacataacatccccacaccatagctacacccaaagtcgctgtacccgtaccgcccgcaaaatggaagtccgacgacgaaaaatcgacgtgctcaaggcccagaaaaacggctacgaatcgggcccaccatctcgacaatcgtcgcagccctcctcaagagcatcgtccagaacccgcaacaaacactcctcgtccaccctgtcgctcagcggactgaccatgaaagtccagaagaaacctgcgggacccccggcgaactccaaaacgccattcctacacatcaagcccgtgcacacgtgctgctccacatcaatgctttcgcgcgattatgacggctccaaccccagcttcaagggcttcaaaaacatcggcatgatcattctcattgtgggaaatctacggctcgcattcgaaaactacctcaaatacggcatttccaacccgttcttcgaccccaaaattactccttccgagtggcagctctcaggcttgctcatagtcgtggcctacgcacatatcctcatggcctacgctattgagagcgctgccaagctgctgttcctctctagcaaacaccactacatggccgtggggcttctgcataccatgaacactttgtcgtccatctcgttgctgtcctacgtcgtctactactacctgcccaaccccgtggcaggcacaatagtcgagtttgtggccgttattctgtctctcaaactcgcctcatacgccctcactaactcggatctccgaaaagccgcaattcatgcccagaagctcgacaagacgcaagacgataacgaaaaggaatccacctcgtcttcctcttcttcagatgacgcagagactttggcagacattgacgtcattcctgcatactacgcacagctgccctacccccagaatgtgacgctgtcgaacctgctgtacttctggtttgctcccacactggtctaccagcccgtgtaccccaagacggagcgtattcgacccaagcacgtgatccgaaacctgtttgagctcgtctctctgtgcatgcttattcagtttctcatcttccagtacgcctaccccatcatgcagtcgtgtctggctctgttcttccagcccaagctcgattatgccaacatctccgagcgcctcatgaagttggcctccgtgtctatgatggtctggctcattggattctacgctttcttccagaacggtctcaatcttattgccgagctcacctgttttggaaacagaaccttctaccagcagtggtggaattcccgctccattggccagtactggactctatggaacaagccagtcaaccagtactttagacaccacgtctacgtgcctcttctcgctcggggcatgtcgcggttcaatgcgtcggtggtggttttctttttctccgccgtcatccatgaactgcttgtcggcatccccactcacaacatcatcggagccgccttcttcggcatgatgtcgcaggtgcctctgatcatggctactgagaaccttcagcatattaactcctctctgggccccttccttggcaactgtgcattctggttcacctttttcctgggacaacccacttgtgcattcctttattatctggcttacaactacaagcagaaccagtagcataacactcatcagtagcctttacagtgataaataatattgacattgacgtaatctgatgattgtagacaaacgtgagacggcattagcgataatattttgacgatgcccacgggcatataataaccattctgtaatagaactttgagcatgttcagaagtaatgagtttcatacataggagtacagatcgtggcgatgtgtaattaccggtaccgcatatgttgaacctgttcgtaaaaggtgccttcaacacagttttgttgccgtcttacctgtgtataagcgttcagcttctggtgtgtagcagaaggaggtgaagttgaggaacaaataatggagacttgcagcagactccgtgcctcaagcaaaaaagatgatccagcagaggatcgaactctgagcctcctcggcactcgtagattgaagccactttgtgagcgagacgtgataaccattacaccactagaccgggggtgaagatcagagcccgtattgtcgtattcaaagttaaaaatggggattttttatattcagaagtgtgtgatttattgaacgccaataacccggcttactacagtaggtatatagctgtaatgaatcattagaataaaaaaaaatccgcctcaaagtagctgaaatgtatccctagaataatgaggggaaaaaactgataaatcaggtgaaatacggcatttagggacaggtaagagttcaattgaatttatgtcgtattatttaagctgtcggagaaaacttgtgtgcttttttgtatggttttcgtgactgtgtacatttctgtacccctgattccgaacatcttatactgacacaagacattggagataaatcatacagtataaagtagctacgtcttacttgtacagtgcttttaattacaatatgccccactatacatacatgtgattcattttataaacccctcctcaggctaattacttctaatgcgtttcgtatatattatctatggaattacaaga

SEQ ID NO: 72 - protein, 526 aa, Yarrowia lipolytica
MEVRRRKIDVLKAQKNGYESGPPSRQSSQPSSRASSRTRNKHSSSTLSLSGLTMKVQKKPAGPPANSKTPFLHIKPVHTCCSTSMLSRDYDGSNPSFKGFKNIGMIILIVGNLRLAFENYLKYGISNPFFDPKITPSEWQLSGLLIVVAYAHILMAYAIESAAKLLFLSSKHHYMAVGLLHTMNTLSSISLLSYVVYYYLPNPVAGTIVEFVAVILSLKLASYALTNSDLRKAAIHAQKLDKTQDDNEKESTSSSSSSDDAETLADIDVIPAYYAQLPYPQNVTLSNLLYFWFAPTLVYQPVYPKTERIRPKHVIRNLFELVSLCMLIQFLIFQYAYPIMQSCLALFFQPKLDYANISERLMKLASVSMMVWLIGFYAFFQNGLNLIAELTCFGNRTFYQQWWNSRSIGQYWTLWNKPVNQYFRHHVYVPLLARGMSRFNASVVVFFFSAVIHELLVGIPTHNIIGAAFFGMMSQVPLIMATENLQHINSSLGPFLGNCAFWFTFFLGQPTCAFLYYLAYNYKQNQ

SEQ ID NO: 73 - DNA, 3947 bp, Yarrowia lipolytica
gagtcgactttccgatacagaagtggaaccagacttgctccacattcatctttcccccatttttctatagtggttcagtggagttgactacttgataacaattggtacatgtaatactgatacatctccgtggatataccatacacattcagtgaccaagcagcgtcgaacatgaccttcaagaatgggttccctgtcatctggagggatgacggctctggtttgtcaagtctgctgaatgcatctacttgtacgttgtagtaacttcctctgtcctccctagaaacgacccttcaatgcagcacagacgataaatcagcgtatatcgtccagtccaactgtgagacctacgatgacctagagaatggttctcttaatctggagtctgcttcttctccacacaactgacagcgtatcccccatggcaccgaagctccagtactgaccgacctttgctacaagtgctgtgtccagagttctctagtaggtacgatacgacagcagcttgatactgtgcttggacctacccttgcatgaggtcttgctgttgtccactgacacaggaccattctgcggggtccactgaaacaagaccattctgacgcgatttctttgtacagatgaagtcagaaatacaagcaatggcaccactcttttggttcgaccatgactgtttttacatccatttccagtaccaaccgcacttttttcatgttcgccccacatttattaatattatgctcttcatgcaccagcaaaataaccgaaacgcgcatatgatagtgggattctcgatttgcccggcagacaaacgccgctaaaatcgccacagtatcgaattttaattgaatacgaacgtcaattccggcttatccttctagcagttgtctcccgcagctcgctccatgactaatcattcacgcgacatgtctcagctaccccggtctggctcatgtaaaaaaagtgtaatcggcttttttccggttgatcacaaccatcaatgacacaacctgtgaatcggaaggcgactgtcgagcgggtcgagccagcagtggaggtggctgactccgagtccgaggccaagaccgacgtccacgttcaccaccatcatcaccaccacaagcgaaaatccgtcaagggcaagattctcaacttcttcacccgaagtcgacgtatcaccttcgtcctcggcgccgtggtcggtgtgatagccgcgggatactacgctgcgccaccggagctcagcattgatatcgatgctcttctcggcgacttgccctcgttcgactttgacgctctatctctcgacaacttgtccatggacagtgtgtcggactttgtacaagacatgaaatcgcggtttccgaccaagattctgcaggaggcggccaagatcgagaagcaccagaaaagcgaacagaaggctgccccttttgctgtgggcaaggctatgaagagcgagggactcaacgccaagtacccggtggtgctggtgcccggcgtcatctccacgggactggagagctggtccctggagggaaccgaggagtgtcccaccgagtcgcacttcagaaagcgaatgtggggctcctggtacatgatccgagtcatgctgctggacaagtactgctggctgcagaacctgatgctggacacagagaccggtctagaccctccccatttcaagctgcgagccgcccagggatttgcctccgccgacttctttatggcaggctactggctgtggaacaagctgctcgagaacctggctgttattggatacgatacggatacaatgtctgctgcggcgtacgactggagactgtcctaccctgatttggagcaccgagacggatacttctccaagctcaaagcttcaatcgaagagactaagcgtatgacaggtgagaagacagttctgacgggccattccatgggctcccaggtcatcttctacttcatgaagtgggctgaggccgagggatatggaggaggaggtcccaactgggtcaatgaccatattgaatcctttgtcgacatttccggctccatgctgggtactcccaagaccctggttgctcttctgtctggagaaatgaaggataccgtgcagctgaacgcgatggctgtgtatggactggagcagttcttctctcgacgagagcgagccgatctgctgcgaacatggggaggaattgcttccatgattcccaagggtggtaaggctatctggggtgatcattctggagcccctgatgacgagcctggccagaatgtcacctttggcaacttcatcaagttcaaggagtccttgaccgagtactctgctaagaacctcaccatggatgaaaccgttgacttcctgtattctcagtctcccgagtggtttgtgaaccgaaccgagggtgcttactcctttggaattgccaagactcgaaagcaggttgagcagaatgagaagcgaccttctacctggagcaaccctctggaagctgctctccccaatgcccccgatctcaagatctactgcttctatggagtcggtaaggataccgagcgagcctactactaccaggatgagcccaatcccgagcagaccaacttgaacgtcagtatcgctggaaacgaccctgatggtgtgcttatgggccagggcgatggaaccgtctcccttgtgacccataccatgtgtcaccgatggaaggacgagaattccaagttcaaccctggtaacgcccaggtcaaggttgtggagatgttgcaccagcctgatcgacttgatattcgaggcggtgctcagactgccgagcatgtggacattctggggcgttctgagttgaacgagatggttctgaaggtggccagtggaaagggaaatgagattgaagagagagtcatctccaacattgatgagtgggtgtggaagattgatctcggcagcaattagagagtccgttttgtagagtaatatgttttgtatatcacactgatggagaagggcgttcgtcctctcatgattccatgtggttgtttaatgaacacgtagaacgacgaacgacgaacgacgaacgacgaacgaacgagcaacaaacgtcatcaacgcgtgttttcccgcaaaatgaagaatgtttcccccacaatatgcgcctttctccagatctgcgcgttctgataaagaatatatttcaattgtttggaataaatcgggaaaattcaggagaaaagaggagaaacagggaaatcactgaaatctactcctgaaaatgttcgacaaaaaacgaattgcttccaatatacaaccactcctgtcacctcctcaacaaaatatattttactccgccaccaatgccaacccatggtgacgccaagattcacaagactgcttggagcggctgaaaagagtgttttgcggtcttctaagcgaccccgagccgctcaaagacccattggaggagatcttctttggccatttcggttctgttggctatcatccgcctccaaaaagcaaccccatttgcttctttttccaacccgttaaaatacctcatctcgcttcacattccagggtttcattttaagcgtagcaccatgcaaagtgcaggtgtagggcgtacaataaagcctgtgagacactctttgacaaggacaggacgctcgtatttttgagacgttgatgttgggggctagtggggcgatttgggggcaataaacggggtaaatctgtgcttttgaggctgtatttttgattccagccttttttaatttattgttatctgctttactacaaatttctgccgtatttttagcccctcaatttcgtctgtcacttctcatgtcgcacaaggctgtgccatcttgcattaggttatccaaccctactgtctcttctctgtctgtctggaatctgaaagcaagccgtgcgactttttgcaacttgcgaggtccgt

SEQ ID NO: 74 - protein, 648 aa, Yarrowia lipolytica
MTQPVNRKATVERVEPAVEVADSESEAKTDVHVHHHHHHHKRKSVKGKILNFFTRSRRITFVLGAVVGVIAAGYYAAPPELSIDIDALLGDLPSFDFDALSLDNLSMDSVSDFVQDMKSRFPTKILQEAAKIEKHQKSEQKAAPFAVGKAMKSEGLNAKYPVVLVPGVISTGLESWSLEGTEECPTESHFRKRMWGSWYMIRVMLLDKYCWLQNLMLDTETGLDPPHFKLRAAQGFASADFFMAGYWLWNKLLENLAVIGYDTDTMSAAAYDWRLSYPDLEHRDGYFSKLKASIEETKRMTGEKTVLTGHSMGSQVIFYFMKWAEAEGYGGGGPNWVNDHIESFVDISGSMLGTPKTLVALLSGEMKDTVQLNAMAVYGLEQFFSRRERADLLRTWGGIASMIPKGGKAIWGDHSGAPDDEPGQNVTFGNFIKFKESLTEYSAKNLTMDETVDFLYSQSPEWFVNRTEGAYSFGIAKTRKQVEQNEKRPSTWSNPLEAALPNAPDLKIYCFYGVGKDTERAYYYQDEPNPEQTNLNVSIAGNDPDGVLMGQGDGTVSLVTHTMCHRWKDENSKFNPGNAQVKVVEMLHQPDRLDIRGGAQTAEHVDILGRSELNEMVLKVASGKGNEIEERVISNIDEWVWKIDLGSN

SEQ ID NO: 75 - DNA, 3632 bp, Yarrowia lipolytica
gccgacaatctctcttcctcattaccgcgctttaatagcgaggagctattagtttgatttcatccaaaccccgcgggacgtattggcgggccaaccaaaaaaagagtcttcatttagcgcgaaaaaaacaagaggttgaaggcaatatttctgctcaattagacgacacatgatctcttgtccaacagcaatacggttcctggacgccgcgggtcacgtgaagggtatccccggcttgtggatctctggacacctgtggatctatcgaccctatggacttagctggtctgtgatcatgcgatgaggccaagacttttgtgcagatgaatcatgtgtctgggtgagacttgggtcggtcttgtagcaaggctggcggggcgccacaccgacggcgaggaagggggggttctcgttgactccggcctccgactgcgagctgtgaaatcgcgcgaccgttcgctggtgccttacagatgcatctaaaaaaagcctcgttgttgctgggtaagtcaaacctcggtaagcacgatgggctgcccagagagagcgagtgcactgcagcccggaaggttgactgaagaaatcttataccgtttatcttctttaatttttttgtcagcgcctttaaactcggcggcttaattagtgaccgttttgggcacctaaaatggcgcacgaggttattgttgaggtcagtaggtgtgagagagtctgagggcgggggccggacaagtgctcacttatatagttgtctcccatgacacgggtctggtaacacgccggcgcctgacacggctcagatttggttcagcttggcacaatttggtttcaagcttgcactttagcagtttgtacactggcagcaagtttgaatttcagtgcatgagaactctatgtataagatgcaaatgacgcacggccctacatatagcggggtctatcgtggcgacggggcaaaaaggatacaagtcgttcttacaagacactctccgcacacacaatggccacactccaccccgaagacgccgcaggacggcccgtgcgacgacgacctcgtccctccagttcgggcggctccagatcgccgtccaccaaacgacactcgatagtgcgggagcatctcggagaagagctcaatgtgcccgacggccaggaaatggacctgggccaggtcaacaagaacctcaatgccgcatacgccaaggccgagaaggactcggacgacgagaaggaaaagaaggaggagggcgtggtggacgagctgccagagaagtattcctaccctcgattctcaaagaacaaccgacgctacagattcaccgacatcaagttcaagccaacaccgtcgattctcgacaagttcgcccacaaggactcggagttctttggcttctacaccctgctgtggatggtgtttgccttctgcgtcttccgaaccggcctgctcaactacacaaacgaaggcatcctgttccggggccagattttcgccattctcagcaaagatctctggaaagtcgcattggtcgatctgggcatgtacctgaccacctatctgtctgtgtttctgcaattggccgtcaagcacggtctggtcgactggaactcgtttggctggatcatccagaacgtgcaccagaccctgttcctcttcttctacctttgggtcgccaagtcgagtaacctgccttggatcggtaacatcttcattgtgcttcatgcctttgtcatgctcatgaaacaacactcgtacgccttctacaatggctacctatggactgtcgaggacgagctctcccacgcaaagcagcgtctcaccgaagacattcctgtttcagagaaggaggatctcaagctggacatcgagttctgcgagacagagctcaaggtccaatccagacacacccctttccccaccaacatcaccttttctaactacttctggtactccatgttcccaacgctcgtctacgaaattgagttccctcgaaccccccgaatcaagtggacatacgtgctggagaaggtcgccgcagtctttggcgtcttcttccttatgatctgggtcgcagagtcgtacctgtatccccctgtggtggctgttattcaaatgcgagacgaacccttctggaacaaggtccgaatctatcccattttcctgtcggacattctgctgccctttgtcattgagtacatgcttgttttctacatcatctgggacgccattctcaacggcattgccgagctcactcgcttcgccgacagagacttttatggcccctggtggaactgtaccagctgggagcagtttagccgagaatggaacattcctgtctaccagttcctcaagcgacacgtctaccactcgtccatctctgctttcaagttctccaagggcgcagctaccctcaccaccttcttgctgtcttctcttgtccacgagctggtcatgtttgccatctttaagaagttccgaggatacctgctgttgctgcagatgacccagctgcccctggccatgctgcagaaaaccaaatggatccaggacagacccgtttttggcaacgctttcttctggttctcgctcatgatcggaccttctctcatgtgttccatgtacctcctcttctaagcacagtcgcttcaccacttgtgcctatagaatgtcaaccactgtcaacttcttgtatggccatcaactatgaacaagtcagttgacagctacaatgcttccactgaagttatcctcccccactgtattttattcttaacactgatcatctacctaacgagacactgtagagccactggtattgtatgaaacttcgccaagagatgccgacgatgaaagtctgagtttaagaaaatcaaaaaggggtaaaaaaaacggtttacgccctccgagccgatactacggagatatcggagttacagacactttgggagcaaagtctgcaatacaagcccacggaactagaggtgggtcgtcagtaaaggccgacgatactcagctggctgcataacttccggattgaaagagagccagtctttcgaacctcggctcagaaaagaagagaaggaaggtacaaacatcatacgatatagagtctttttagcacggcggtcagttggattgcaaagtggaagggcaagagtactgctgccttgtttaattggtatccactatgcattttgctttttgcaagtggaactgggagaacttgtagccttgggttgatgcggtcggagattagagttgatctgaaaaataaaaaataaaaaaacgagatgcggattgtggaagctgtagaggatatgtgactacaagagtagtatctacgagtgtgtacttgctgcgaaagttgatttcgaacgaaccagcttcattgactaaacagtgatgattttcgagacagcggaaaacttcaagccagtatgcagcatttaggctcatcggcacctattttagccgcaactagccgcttctttctgtaacatacgatgatttgtcaggtacagtacagtaccgttttaagcacctgaactagcggtggcctacgtgcacatgctcgtattgtagtaccgagttaaagattgttttggagtctagagttgtatttgtcattgtttta

SEQ ID NO: 76 - protein, 543 aa, Yarrowia lipolytica
MATLHPEDAAGRPVRRRPRPSSSGGSRSPSTKRHSIVREHLGEELNVPDGQEMDLGQVNKNLNAAYAKAEKDSDDEKEKKEEGVVDELPEKYSYPRFSKNNRRYRFTDIKFKPTPSILDKFAHKDSEFFGFYTLLWMVFAFCVFRTGLLNYTNEGILFRGQIFAILSKDLWKVALVDLGMYLTTYLSVFLQLAVKHGLVDWNSFGWIIQNVHQTLFLFFYLWVAKSSNLPWIGNIFIVLHAFVMLMKQHSYAFYNGYLWTVEDELSHAKQRLTEDIPVSEKEDLKLDIEFCETELKVQSRHTPFPTNITFSNYFWYSMFPTLVYEIEFPRTPRIKWTYVLEKVAAVFGVFFLMIWVAESYLYPPVVAVIQMRDEPFWNKVRIYPIFLSDILLPFVIEYMLVFYIIWDAILNGIAELTRFADRDFYGPWWNCTSWEQFSREWNIPVYQFLKRHVYHSSISAFKFSKGAATLTTFLLSSLVHELVMFAIFKKFRGYLLLLQMTQLPLAMLQKTKWIQDRPVFGNAFFWFSLMIGPSLMCSMYLLF

SEQ ID NO: 77 - DNA, 4103 bp, Yarrowia lipolytica
gattccgccaagtgagactggcgatcgggagaagggttggtggtcatgggggatagaatttgtacaagtggaaaaaccactacgagtagcggatttgataccacaagtagcagagatatacagcaatggtgggagtgcaagtatcggaatgtactgtacctcctgtactcgtactcgtacggcactcgtagaaacggggcaatacgggggagaagcgatcgcccgtctgttcaatcgccacaagtccgagtaatgctcgagtatcgaagtcttgtacctccctgtcaatcatggcaccactggtcttgacttgtctattcatactggacaagcgccagagttaagcttgtagcgaatttcgccctcggacatcaccccatacgacggacacacatgcccgacaaacagcctctcttattgtagctgaaagtatattgaatgtgaacgtgtacaatatcaggtaccagcgggaggttacggccaaggtgataccggaataaccctggcttggagatggtcggtccattgtactgaagtgtccgtgtcgtttccgtcactgccccaattggacatgtttgtttttccgatctttcgggcgccctctccttgtctccttgtctgtctcctggactgttgctaccccatttctttggcctccattggttcctccccgtctttcacgtcgtctatggttgcatggtttcccttatacttttccccacagtcacatgttatggaggggtctagatggaggcctaattttgacgtgcaaggggcgaattggggcgagaaacacgtcgtggacatggtgcaaggcccgcagggttgattcgacgcttttccgcgaaaaaaacaagtccaaatacccccgtttattctccctcggctctcggtatttcacatgaaaactataacctagactacacgggcaaccttaaccccagagtatacttatataccaaagggatgggtcctcaaaaatcacacaagcaacgacgccatgaaccccaacaacactggcaccattgaaatcaacggtaaggagtacaacaccttcaccgagccccccgtggccatggctcaggagcgagccaagacctccttccccgtgcgagagatgacctacttcctcgacggtggcgagaagaacaccctcaaaaacgagcagatcatggaggagattgagcgagaccctcttttcaacaacgacaactactacgatctcaacaaggagcagatccgagagctcaccatggagcgagtcgccaagctgtctctgtttgtgcgtgatcagcccgaggacgacatcaagaagcgatttgctctcattggtatcgccgatatgggaacctacacccgacttggtgtccactacggcctcttctttggcgccgtccgaggtaccggaactgccgagcagtttggccactggatctccaagggagccggagacctgcgaaagttctacggatgtttctccatgaccgagctgggccatggctccaacctggctggtctcgagaccaccgccatctacgatgaggagaccgacgagttcatcatcaacacccctcacattgccgccaccaagtggtggattggaggagccgcccacaccgccacccacactgtcgtgttcgcccgactcattgtcaagggcaaggactacggtgtcaagacctttgttgtccagctgcgaaacatcaacgaccacagcctcaaggtcggtatctctattggtgatatcggaaagaagatgggccgagacggtatcgataacggatggatccagttcaccaacgtgcgaatcccccgacagaacctgctcatgaagtacacaaaggtcgaccgagagggtaacgtgacccagcctcctctggctcagcttacctacggttctcttatcactggtcgagtctccatggcctctgattctcaccaggtcggaaagcgattcatcaccattgctctgcgatacgcctgcattcgacgacagttctccaccacccccggccagcccgagaccaagatcatcgactacccctaccatcagcgacgacttctgcctcttctggcctatgtctatgctcttaagatgactgccgatgaggttggagctctcttctcccgaaccatgcttaagatggacgacctcaagcccgacgacaaggccggcctcaatgaggttgtttccgacgtcaaggagctcttctccgtctccgccggtctcaaggccttctccacctgggcttgtgccgacgtcattgacaagacccgacaggcttgcggtggccacggttactctggatacaacggtttcggccaggcctacgccgactgggttgtccagtgcacctgggagggtgacaacaacattctcaccctttctgccggccgagctcttatccagtctgccgttgctctgcgaaagggcgagcctgttggtaacgccgtttcttacctgaagcgatacaaggatctggccaacgctaagctcaatggccgatctctcaccgaccccaaggtcctcgtcgaggcctgggaggttgctgccggtaacatcatcaaccgagccaccgaccagtacgagaagctcattggcgagggtcttaacgccgaccaggcctttgaggttctgtctcagcagcgattccaggccgccaaggtccacacacgacgacacctcattgccgctttcttctcccgaattgacaccgaggctggcgaggccatcaagcagcccctgcttaacctggctctgctgtttgccctgtggtccatcgaagaggactctggtctgttcctgcgagagggcttcctcgagcccaaggatatcgacaccgtcaccgagctcgtcaacaagtactgcaccactgtgcgagaggaggtcattggctacaccgatgccttcaacctgtccgactacttcatcaacgctcctattggatgctacgatggtgacgcttaccgacactacttccagaaggtcaacgagcagaaccctgcccgagacccccgacctccttactacgcctctactctcaagcccttccttttccgagaggaggaggatgatgacatttgcgagcttgatgaggaatagacaagcgggtatttattgtatgaataaagattatgtattgattgcaaaaaagtgcatttgtagatgtggtttattgtagagagtacggtatgtactgtacgaacattaggagctacttctacaagtagattttcttaacaagggtgaaatttactaggaagtacatgcatatttcgttagtagaatcacaaaagaaatgtacaagcacgtactacttgtactccacaatgtggagtgggagcaaaaaaattggacgacaccggaatcgaaccggggacctcgcgcatgctaagcgcatgtgataaccaactacaccagacgcccaagaactttcttggtgattatggaatacgtggtctgctatatctcaattttgctgtaatgaatcattagaattaaaaaaaaaaccccatttttgtgtgattgtcggccaagagatggaacaggaagaatacgtgaacaagcgagcacgaatgccatatgctcttctgaacaaccgagtccgaatccgatttgtgggtatcacatgtctcaagtagctgaaatgtatttcgctagaataaaataaatgagattaagaattaaaaatattggaatatattttcctagaatagaaactttggattttttttcggctattacagtctgaactggacaaacggctgactatatataaatattattgggtctgttttcttgtttatgtcgaaattatctgggttttactactgtgtcgtcgagtatagagtggcctgactggagaaaatgcagtagtatggacagtaggtactgccagccagagaagtttttggaattgatacttgagtcatttttccattccccattccccattccaacacaatcaactgtttctgaacattttccaaaacgcggagatgtatgtcacttggcactgcaagtctcgattcaaaatgcatctctttcagaccaaagtgtcatcagctttgtttggccccaaattaccgcaaatacttgtcga

SEQ ID NO: 78 - protein, 700 aa, Yarrowia lipolytica
MNPNNTGTIEINGKEYNTFTEPPVAMAQERAKTSFPVREMTYFLDGGEKNTLKNEQIMEEIERDPLFNNDNYYDLNKEQIRELTMERVAKLSLFVRDQPEDDIKKRFALIGIADMGTYTRLGVHYGLFFGAVRGTGTAEQFGHWISKGAGDLRKFYGCFSMTELGHGSNLAGLETTAIYDEETDEFIINTPHIAATKWWIGGAAHTATHTVVFARLIVKGKDYGVKTFVVQLRNINDHSLKVGISIGDIGKKMGRDGIDNGWIQFTNVRIPRQNLLMKYTKVDREGNVTQPPLAQLTYGSLITGRVSMASDSHQVGKRFITIALRYACIRRQFSTTPGQPETKIIDYPYHQRRLLPLLAYVYALKMTADEVGALFSRTMLKMDDLKPDDKAGLNEVVSDVKELFSVSAGLKAFSTWACADVIDKTRQACGGHGYSGYNGFGQAYADWVVQCTWEGDNNILTLSAGRALIQSAVALRKGEPVGNAVSYLKRYKDLANAKLNGRSLTDPKVLVEAWEVAAGNIINRATDQYEKLIGEGLNADQAFEVLSQQRFQAAKVHTRRHLIAAFFSRIDTEAGEAIKQPLLNLALLFALWSIEEDSGLFLREGFLEPKDIDTVTELVNKYCTTVREEVIGYTDAFNLSDYFINAPIGCYDGDAYRHYFQKVNEQNPARDPRPPYYASTLKPFLFREEEDDDICELDEE

SEQ ID NO: 79 - DNA, 4247 bp, Saccharomyces cerevisiae
tcactcaaccacctccaaaaaataacaggttcatctaaagtaaaagactttaacttgctcttagtttccaaattaaatatctgcacgatagtaccatttgctctaacggaaataaccatctgagatggatgcatgatagcagaatcaccgcccatattcttccttgtcacttcattgcctttggccaaatccacgattgcaacagagtttgtaccgtcctttgtttctctaacagtgacgaagtggtcactctcgaaagtagttgatctgaagtcaaggaattgaggggaaattcctaaggacatcagatcgaccaattcggtaaattcaatgggtaggtcactcattggttagaactttcgtgataatttatttttatagttgaatatcttctttctctctcaactctgatccggattgtcgaggtttcaataagttactctgaacaactaatcaaaatatctccttatttctgtagattccttcagttccactttttacttttcttaattctctttgtatttattcctagcgacgaaaaatgcgagatctcgaccaaaaaaagggggtagggtaataaaattaaccctattattttttaactttaaaacctataatgtgctaatattttattataaacctcctttttttgcgttcaaaccctgacacattttaagccctatatttacggtattagttgattaaactccgaagcgaaaggaattcggtcattagcggctaatagccgttggggtaaatcacctacaagcaagtacacaagagaacgttggcgttgttaagtcaaagcactaatacattggggctttaagagtgtttataaaggtctaacctgtaaaaattatttaaacaacttgaacaggccttaaagttttcctcattccgctcatcatcactaatattgctctccgtttttgaatacacacttgacactaataagtatcacagaaaaaaagaaaatataataaattagtattgcgatatgacgagacgtactactattaatcccgattcggtggttctgaatcctcaaaaatttatccagaaagaaagggcggattcgaaaatcaaagttgaccaagttaacacatttttagagtcatccccggagaggagaactctgacgcacgccttaatagaccaaatagtgaatgatcctatattgaaaactgatacggactattacgatgctaaaaaaatgcaagagagagaaattactgccaaaaaaatagctaggcttgctagttatatggagcacgatatcaaaacagtgcgcaaacactttcgcgacactgacctgatgaaagagttgcaagcaaatgatccagacaaagcttcgcctttaacaaacaaagacctttttatattcgataagagattgtcacttgtagcaaatattgatcctcaattgggtacgcgcgtgggtgtacacttggggctatttggtaattgtatcaagggcaatggtactgatgagcaaatccggtattggttgcaggagagaggtgccactttgatgaaaggtatatatggctgttttgcaatgactgagttaggacatggttccaatgttgcccagctgcagactagggctgtgtacgataagcaaaatgatacttttgtaattgatacacctgatctaactgccaccaaatggtggattggtggggctgcccattctgccacgcacgctgccgtgtacgccagattgatcgttgaaggtaaagactacggtgtaaaaacattcgttgttcctctgagagacccttcgactttccaactgttagctggtgtttccataggggatattggagcgaagatgggtcgtgacggtattgataatggctggatccagttcagaaacgtagttatccctagagaatttatgctaagtagatttaccaaagttgtccgttctccagatggttcagtcaccgtcaaaactgagccacaattggatcaaatttctggttatagtgcattgttaagtggtagagttaacatggtcatggattcatttaggtttggctccaaatttgctactattgctgtacgttacgcggttggtcgtcagcaattcgcacctagaaagggattgtctgaaacacaattaatcgactatccccttcaccaatatcgtgttttaccacaattgtgtgttccatatttggtgtcacctgtagcttttaagttaatggacaactattattccactttggacgagttatacaacgcttcctcatctgcatacaaagctgctctggttaccgtgagtaaaaagttgaagaatttatttattgatagcgccagcttgaaagccaccaatacttggttaattgctacactgattgatgagttgagacagacttgcggaggacatgggtattcacagtataacggatttggtaaaggctatgacgactgggtggttcagtgcacatgggagggtgataataatgttttatctttaacttcagcaaaatcaatattgaaaaaatttatcgattcagccacaaagggtagatttgacaacacactggatgtggactcattctcttacttaaaacctcagtacataggatctgtggtttctggagaaataaagagtggtttaaaggagttgggtgattatactgaaatttggtctatcaccttaatcaaattactggcacatattggtactttagttgaaaaatcaagaagtattgatagcgtttctaagcttttagtcttagtatccaaatttcatgccttgcgctgcatgttgaaaacctattacgacaagttaaactctcgtgattcacatatttccgatgaaattacaaaggaatctatgtggaatgtttataagttattttccttgtattttattgacaagcattccggagaattccaacaattcaagatcttcactcctgatcagatctctaaagttgtgcagccacaactattggctcttttgccaattgtgaggaaagactgtataggtctgacagactcctttgaattacctgacgcgatgttaaattctcctataggttactttgatggcgatatctatcacaattacttcaatgaagtttgccgcaataatccagtggaggcagatggggcagggaagccttcttatcatgcgctgttgagcagcatgctcggtagaggtttcgaatttgaccaaaagttaggtggtgcagctaatgcggaaattttatcgaaaataaacaagtgagtagaggtttcctgttttccttcgaaccctctgttttgcgacttttgtttcaattcaactagtgtcgccaagttttaacaaaaagttacaaaatcctagtgagaggccatcttatgtgcataacggtactctctatctatttacatatctaatactattcacataactatgacgaatcaatgacatgactacatttaccaatgtatagtagtaaaaaaattgccatctcctagaatcgaaccagggtttcatcggccacaacgatgtgtactaaccactatactaagatggcaggtaattattgagtgtttactcaatatctcagaattatatacataacaaataataaatgtgctgtgttggcacttgttggaatgaagctctaaaacatcatgatgattgtgtggattctgatgtaattgttgggattccatttttaataaggcaatatgttggaacgagagtaattaatagtgacatgagttgctatggtaacaatctaatgcttacatcgtatattaatgtacacatcgtatacgtttaagtgtgattgcgcctattgcagaaggaatgttaaacgagaagctcagacaatactgaagctgtgttaaagacctattagctgaacatgatatggtaggtacatatatgaggaatatgagtcgtcacatcaatgtatagtaactaccggaatcactattatattggtcatgattaatatgaccaatcggcgtgtgttttatatacctctcttatttagtataagaagatcagtactcacttcttcattaatactaatttttaacctctaattatcaacacaataatattaggtatgtagatatactagaagttctcctccaggatttaggaatccataaaagggagtctgcaattctacacaattctataaatattatcatcatcgttttatatgttaatattcattgatcctattacattatcaatccttgcgtttcagcttccactaatttagatg

SEQ ID NO: 80 - protein, 748 aa, Saccharomyces cerevisiae
MTRRTTINPDSVVLNPQKFIQKERADSKIKVDQVNTFLESSPERRTLTHALIDQIVNDPILKTDTDYYDAKKMQEREITAKKIARLASYMEHDIKTVRKHFRDTDLMKELQANDPDKASPLTNKDLFIFDKRLSLVANIDPQLGTRVGVHLGLFGNCIKGNGTDEQIRYWLQERGATLMKGIYGCFAMTELGHGSNVAQLQTRAVYDKQNDTFVIDTPDLTATKWWIGGAAHSATHAAVYARLIVEGKDYGVKTFVVPLRDPSTFQLLAGVSIGDIGAKMGRDGIDNGWIQFRNVVIPREFMLSRFTKVVRSPDGSVTVKTEPQLDQISGYSALLSGRVNMVMDSFRFGSKFATIAVRYAVGRQQFAPRKGLSETQLIDYPLHQYRVLPQLCVPYLVSPVAFKLMDNYYSTLDELYNASSSAYKAALVTVSKKLKNLFIDSASLKATNTWLIATLIDELRQTCGGHGYSQYNGFGKGYDDWVVQCTWEGDNNVLSLTSAKSILKKFIDSATKGRFDNTLDVDSFSYLKPQYIGSVVSGEIKSGLKELGDYTEIWSITLIKLLAHIGTLVEKSRSIDSVSKLLVLVSKFHALRCMLKTYYDKLNSRDSHISDEITKESMWNVYKLFSLYFIDKHSGEFQQFKIFTPDQISKVVQPQLLALLPIVRKDCIGLTDSFELPDAMLNSPIGYFDGDIYHNYFNEVCRNNPVEADGAGKPSYHALLSSMLGRGFEFDQKLGGAANAEILSKINK

SEQ ID NO: 81 - DNA, 588 bp, Saccharomyces cerevisiae
atcgctccctccttcttgaatacgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatg

SEQ ID NO: 82 - DNA, 520 bp, Saccharomyces cerevisiae
ctagtcttctaggcgggttatctactgatccgagcttccactaggatagcacccaaacacctgcatatttggacgacctttacttacaccaccaaaaaccactttcgcctctcccgcccctgataacgtccactaattgagcgattacctgagcggtcctcttttgtttgcagcatgagacttgcatactgcaaatcgtaagtagcaacgtctcaaggtcaaaactgtatggaaaccttgtcacctcacttaattctagctagcctaccctgcaagtcaagaggtctccgtgattcctagccacctcaaggtatgcctctccccggaaactgtggccttttctggcacacatgatctccacgatttcaacatataaatagcttttgataatggcaatattaatcaaatttattttacttctttcttgtaacatctctcttgtaatcccttattccttctagctatttttcataaaaaaccaagcaactgcttatcaacacacaaacactaaatcaaaatg

SEQ ID NO: 83 - DNA, 670 bp, Saccharomyces cerevisiae
tcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacgggcacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaatg

SEQ ID NO: 84 - DNA, 278 bp, Saccharomyces cerevisiae
attgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgtttcgaaag

SEQ ID NO: 85 - DNA, 282 bp, Saccharomyces cerevisiae
ttttcctttgtcgatatcatgtaattagttatgtcacgcttacattcacgccctcctcccacatccgctctaaccgaaaaggaaggagttagacaacctgaagtctaggtccctatttattttttttaatagttatgttagtattaagaacgttatttatatttcaaatttttcttttttttctgtacaaacgcgtgtacgcatgtaacattatactgaaaaccttgcttgagaaggttttgggacgctcgaaggctttggccggcttggaaccgtttaacc

SEQ ID NO: 86 - DNA, 288 bp, Saccharomyces cerevisiae
agcttttgattaagccttctagtccaaaaaacacgtttttttgtcatttatttcattttcttagaatagtttagtttattcattttatagtcacgaatgttttatgattctatatagggttgcaaacaagcatttttcattttatgttaaaacaatttcaggtttaccttttattctgcttgtggtgacgcgtgtatccgcccgctcttttggtcacccatgtatttaattgcataaataattcttaaaagtggagctagtctatttctatttacatacctctcattt

SEQ ID NO: 87 - DNA, 4034 bp, Yarrowia lipolytica
tgagtataatccgtctccgttcccacttgaggggaggactttggacgggtttcggtgcaacctcgtcaggtgaataatcatgattcatttgcgtgatctggctgggacaaaatacaagtgcttgtatatggtataccaggaggaccacttatgagaatgccaaagaaaattgaaggggaaaataaatgctgccacccgatactcgaaatataccattggacgtgttttccacataggtaatcgccactccatacccgagatctgaagtgaaacattcgtgttctgtagccgttagcccacccccacacactgagacactcggctgatgtttcgaaattcatgctactgtacggtacggacttaacacttatccccattgtcctgggactactacattgtagtatactctacacatccaacaagcaatactttttcgtacaagtaggtagttgtgcgagtactcgtacaatggaaaaacattatacatgaagcagggcgtgcgtcggtctgtagggtgaaatagggagccgacagtgcatttccagcattctagattgatctgaatcaataatttgtgggggaaattggcactagtgctttgtttattagctatttgacagtctgatgatgagaaagtcgttcttcggcttagcccaaccccacactcactacgcacctggcacagggtggtgcatataatatgcaagtacggagatgtactaacgctagccaggagagagtgacgaaacgtagccatccgttgagcgggcagacgtgggagtaatatagtcgtttagggcacgtgattgcgaccccagtggtatcatctttgtctcccaaactcttcgtaatgcccacccaggacgatcctctctgaactttactaccccacattcccgagactataaagtatagacggctcctacaaaagagagcaggtggaataacacactattgaccgacaatgacaaccaacacattcaccgatcctcctgtggagatggccaaggagcgaggtaagactcaattcactgtccgagatgtgaccaacttcctcaatggtggagaagaagagacccagattgtcgagaagatcatgagcagtattgaacgtgatccagtactgtctgtcactgctgactacgactgcaaccttcagcaggcccgaaaacagaccatggagcgggtggctgctctgtcgccttatctggtcaccgatactgagaagctatctctgtggcgtgcgcaactgcatggaatggttgatatgtctactcgtacgcggttgtcgatccacaacaacctgttcattggttccatcaggggatctggtactcctgaacagttcaagtactgggtcaagaagggagcggtggctgttaagcagttctatggatgctttgccatgacagagttgggccatggaagcaacctcaagggactagagacaaccgccacttatgaccaggacagtgaccagttcattatcaacactcctcatattggtgctaccaagtggtggattggcggtgcagcccacacttccacccattgtgtttgtttcgcgaaactgattgtgcatggcaaggactatggtactcgaaactttgtggtacctctccgaaatgtccacgatcacagtctcaaggtcggtgtttcaattggagacattggaaagaagatgggcagagatggtgttgacaatggctggatccagttcaccaatgttcgaatccccagacagaacatgctaatgagatatgccaaggtgtctgatactggagtggtaaccaaacccgctcttgaccaactcacttatggagccctcattcgaggtcgagtgtccatgattgccgactcgttccacgtctccaaacgattcctcacaattgctcttcggtacgcttgtgtccgacgacagtttggaacctctggagacactaaggagaccaagatcatcgactacccttaccaccagcgacgattgctgcctcttctggcctactgctacgctatgaagatgggtgctgatgaggctcagaagacttggattgagaccaccgatcgaattctggctctcaatcccaacgaccccgcccagaagaacgatctggagaaggccgtcaccgacacaaaggagctgtttgctgcgtctgcaggaatgaaggcatttaccacgtggggatgtgccaaaatcattgatgagtgccgacaggcctgtggaggtcatggatactctggatataacggatttggccagggctacgctgactgggttgtccagtgtacctgggaaggagacaacaacgttctgtgtctgtcaatgggccgagggctggttcagtcagctctacagattttggctggaaagcacgtcggtgcttctattcagtacgtaggagacaagtctaaaatctcccagaacggccagggtacccccagagagcaacttctgtcccccgagtttctagtagaagctttcagaacggcttctcgaaacaacattctcagaaccaccgataaataccaagagcttgtcaaaactctcaatcccgaccaggcctttgaggagctgtctcagcagagattccagtgtgctcgaatccacacacgacagcatcttatctcttcattctatgcccgaattgccactgccaaagacgatatcaagccccatctgctgaaactggccaatctgtttgccctctggtcaattgaggaggacactggaatcttcctgcgggagaacatcctcacccctggagacattgacctgatcaacagtcttgtggacgagctctgtgttgcagttcgagatcaggtaattggactcactgatgcctttggtctctctgacttcttcattaacgctcccatcggctcctacgatggtaatgtttacgaaaagtactttgccaaggtcaaccagcaaaaccccgctactaaccctcgtcctccctactacgagtcgactctcaagcccttcttgttccgagaagaggaggacgatgaaatttgcgatctcgatgagtgatgttagtaattatgagatacattctttgcaatgatgagggctcttgtagagtattctgctgtaaaggagatcttgaattttgttgttgaatttgtgtaattgtcttatgattattgtcttgtgtagcgcttttcaagcctgcaggaaaagtgaaatgaacatgttattgccatatcatccgttattggtgtttagttcctattgggcttgattaccaacgatccgggtcgaaagacccctccgtttaggggtggtaagtctcaagtggctgaacacgtgcatgcactgtacatcgccgagaccactcaacaccatcaagggctactgtattcgtagagcatacggcgttcaaggcttcaccgataattagagctcagcgcaatcaattatttgaaggcttaatggcactgtcagctgcttgatcttgcacgcaattactacaatatatagtccaacataataacataattagaattatgatcatccgtatcaatcatacacaaaatccttacacctaatctcgtagctcctccacaaatcccacaacgcgtcctgcctccacaattctaagctcctcatctgcagactcgtgcagaccaagccaggtagcgtcctgaacaggcagttcatttgtccaggtctggaaatcagccacacgcgagccagtagtcagcttgcatggaccgagagcacgcgactcgttgtcaaaataatccacctgcatcacctgttggaccatctcttgtaatcgagccaggtcagcagcgatgtcaatcttggcgctgtaaatgtcagacagcaccgagactacagcatgccaaggaatggaatggggagcaatattgtctctctgaccactcacatgacgaacccatccttcaatagctcgcaaactggcagccaggtctccctcatggaagccataatccttggcaaagcccagaggcacaaaacgagccaggtcaacaatgcagacatgcagccaactggccaggaagtt

SEQ ID NO: 88 - protein, 677 aa, Yarrowia lipolytica
MAKERGKTQFTVRDVTNFLNGGEEETQIVEKIMSSIERDPVLSVTADYDCNLQQARKQTMERVAALSPYLVTDTEKLSLWRAQLHGMVDMSTRTRLSIHNNLFIGSIRGSGTPEQFKYWVKKGAVAVKQFYGCFAMTELGHGSNLKGLETTATYDQDSDQFIINTPHIGATKWWIGGAAHTSTHCVCFAKLIVHGKDYGTRNFVVPLRNVHDHSLKVGVSIGDIGKKMGRDGVDNGWIQFTNVRIPRQNMLMRYAKVSDTGVVTKPALDQLTYGALIRGRVSMIADSFHVSKRFLTIALRYACVRRQFGTSGDTKETKIIDYPYHQRRLLPLLAYCYAMKMGADEAQKTWIETTDRILALNPNDPAQKNDLEKAVTDTKELFAASAGMKAFTTWGCAKIIDECRQACGGHGYSGYNGFGQGYADWVVQCTWEGDNNVLCLSMGRGLVQSALQILAGKHVGASIQYVGDKSKISQNGQGTPREQLLSPEFLVEAFRTASRNNILRTTDKYQELVKTLNPDQAFEELSQQRFQCARIHTRQHLISSFYARIATAKDDIKPHLLKLANLFALWSIEEDTGIFLRENILTPGDIDLINSLVDELCVAVRDQVIGLTDAFGLSDFFINAPIGSYDGNVYEKYFAKVNQQNPATNPRPPYYESTLKPFLFREEEDDEICDLDE

SEQ ID NO: 89 - DNA, 4103 bp, Yarrowia lipolytica
tagtgtttttgttggtttttatttgatttgttggtttaatatttattttattttaatatttattttattttatttcaattaattattttatttatttgcttaaatcaaatattttggaccaataaaaaccgacacagttttcagacatgtttgtagctcatatgtatcaggtgtggttgtggggtatcaaagccgaggttggcaaattttactggggccttcagaacggtaaaaatagaccaatcagaattctgaaaagcacatcttgatctcctcattgcggggagtccaacggtggtcttattcccccgaatttcccgctcaatatcgttccagaccgacccggacacagtgcttaacgccgttccgaaactctaccgcagatatgctccaacggactgggctgcatagatgtgatcctcggcttggagaaatggataaaagccggccaaaaaaaaagcggaaaaaagcggaaaaaaagagaaaaaaaatcgcaaaatcggaaaaatagggggaaaagacgcaaaaacgcaaggaggggggagtatatgacactgataagcaagctcacaacggttcctcttatttttttcctcatcttctgcctaggttcccaaaatcccagatgcttctctccagtgccaaaagtaagtaccccacaggttttcggccgaaaattccacgtgcagcaacgtcgtgtggggtgttaaaatgtgggggggggaccaggacaagaggctcttgtgggagccgaatgagagcacaaagcgggcgggtgtgataagggcatttttgcccattttcccttctcctgtctctccgacggtgatggcgttgtgcgtcctctatttctttttatttctttttgttttatttctctgactaccgatttggtttgatttcctcaaccccacacaaataagctcgggccgaggaatatatatatacacggacacagtcgccctgtggacaacacgtcactacctctacgatacacacaatgatctcccccaacctcacagctaacgtcgagattgacggcaagcagtacaacaccttcacagagccacccaaggcgctcgccggcgagcgagccaaggtcaagttccccatcaaggacatgacggagtttctgcacggtggcgaggagaacgtgaccatgatcgagcgactgatgacggagctcgagcgagaccccgtgctcaacgtgtcgggcgactacgacatgcccaaggagcagctgcgagagacggccgtggcgcgaattgcggcgctgtccggccactggaagaaggacacagaaaaggaggcgctgctgcggtcccagctgcacggcattgtggacatgggcacccgaatccgactcggtgtgcacacgggcctgttcatgggcgccatccggggttccggcaccaaggagcagtacgactactgggtgcgaaagggcgccgcggacgtcaagggcttctacggctgctttgctatgaccgagctgggccatggctccaacgtggccggtcttgagaccaccgccacctacatccaggacacggacgagttcatcatcaacacccccaacactggagccaccaagtggtggattggaggagccgcccactcggccacccacaccgcctgctttgctcgtctgcttgtcgacggcaaggactacggcgtcaagatctttgttgtccagctgcgagacgtctcttctcactctctcatgcccggcatcgctctcggcgacattggaaagaagatgggccgagacgccatcgacaacggctggatccagttcaccaatgtgcgaatcccccgacagaacatgctcatgaagtacgccaaggtctcgtctaccggcaaggtgtcgcagcctcctctggcccagctcacctacggcgctctcattggcggccgagtcaccatgattgccgactccttctttgtctcccagcgattcatcaccattgctctgcgatacgcctgtgtgcgacgacagtttggcaccacccccggccagcccgagactaagatcatcgactacccctaccatcagcgacgtctgctgcctcttctggccttcacctacgccatgaagatggccgccgaccagtcccagattcagtacgatcagaccaccgatctgctgcagaccatcgaccctaaggacaagggcgctctgggcaaggccattgtcgacctcaaggagctgtttgcctcttctgctggtctcaaggccttcaccacctggacctgtgccaacatcattgaccagtgccgacaggcctgcggtggccacggctactctggctacaacggctttggccaggcctacgccgactgggttgtccagtgcacctgggagggtgacaacaacgtcctgtgtctgtccatgggccgaggtctcatccagtcgtgtctgggccaccgaaagggtaagcctctgggctcttctgtcggctacctggctaacaagggtcttgagcaggctactctgagcggccgagacctcaaggaccccaaggttctcatcgaggcctgggagaaggtcgccaacggcgccatccagcgggccactgacaaatttgtcgagctcaccaagggcggcctctctcctgaccaggcctttgaggagctgtcgcagcagcgattccagtgtgccaagatccacacccgaaagcacctggtgactgccttctacgagcgaatcaacgcctctgcgaaggccgacgtcaagccttacctcatcaacctcgccaacctcttcactctgtggtccattgaggaggactctggtctcttcctgcgagagggtttcctgcagcccaaggacattgaccaggtgactgagctggtgaaccactactgcaaggaggttcgagaccaggttgccggctacaccgatgcctttggtctgtctgactggttcatcaacgctcccattggaaactacgatggtgacgtttacaagcattactttgccaaggttaaccagcagaaccctgctcagaacccccgacctccttactatgagagcactcttcgacctttcctgttccgagaggatgaggatgacgacatttgcgagctggacgaggaatagatggagcgtgtgttctgagtcgatgttttctatggagttgtgagtgttagtagacatgatgggtttatatatgatgaatgaatagatgtgattttgatttgcacgatggaattgagaactttgtaaacgtacatgggaatgtatgaatgtgggggttttgtgactggataactgacggtcagtggacgccgttgttcaaatatccaagagatgcgagaaactttgggtcaagtgaacatgtcctctctgttcaagtaaaccatcaactatgggtagtatatttagtaaggacaggagttgagagaggaaagttgccattctttggagtcccagaaacgtattttcgcgttccaagatcaaattagtagagtaatacgggcacgggaatccattcatagtctcaattttcccataggtgtgctacaaggtgttgagatgtggtacagtaccaccatgattcgagataaagagcccagaagtcattgatgaggtcaagaaatacacagatctacagctcaatacaatgaatatcttctttcatattcttcaggtgacaccaagggtgtctattttccccagaaatgcgtgaaaaggcgcgtgtgtagcgtggagtatgggttcggttggcgtatccttcatatatcgacgaaatagtagggcaagagatgacaaaaagtatctatatgtagacagcgtagaatatggatttgattggtataaattcatttattgcgtgtctcacaaatactctcgataagttggggttaaactggagatggaacaatgtcgatatctcgacatattttgatatttgtactgttgatagtgataaaaagtagaccgttcgaatctcgacaaggagaagagtccaatgaaataggttccatcatcattcgtcatagttaaacgccgctggttgccattactatccgtcttgactacaaccccaactcagcctagaccacagcgaagagaatcagtttggagaccgaaaatgagc

SEQ ID NO: 90 - protein, 700 aa, Yarrowia lipolytica
MISPNLTANVEIDGKQYNTFTEPPKALAGERAKVKFPIKDMTEFLHGGEENVTMIERLMTELERDPVLNVSGDYDMPKEQLRETAVARIAALSGHWKKDTEKEALLRSQLHGIVDMGTRIRLGVHTGLFMGAIRGSGTKEQYDYWVRKGAADVKGFYGCFAMTELGHGSNVAGLETTATYIQDTDEFIINTPNTGATKWWIGGAAHSATHTACFARLLVDGKDYGVKIFVVQLRDVSSHSLMPGIALGDIGKKMGRDAIDNGWIQFTNVRIPRQNMLMKYAKVSSTGKVSQPPLAQLTYGALIGGRVTMIADSFFVSQRFITIALRYACVRRQFGTTPGQPETKIIDYPYHQRRLLPLLAFTYAMKMAADQSQIQYDQTTDLLQTIDPKDKGALGKAIVDLKELFASSAGLKAFTTWTCANIIDQCRQACGGHGYSGYNGFGQAYADWVVQCTWEGDNNVLCLSMGRGLIQSCLGHRKGKPLGSSVGYLANKGLEQATLSGRDLKDPKVLIEAWEKVANGAIQRATDKFVELTKGGLSPDQAFEELSQQRFQCAKIHTRKHLVTAFYERINASAKADVKPYLINLANLFTLWSIEEDSGLFLREGFLQPKDIDQVTELVNHYCKEVRDQVAGYTDAFGLSDWFINAPIGNYDGDVYKHYFAKVNQQNPAQNPRPPYYESTLRPFLFREDEDDDICELDEE

SEQ ID NO: 91 - DNA, 4706 bp, Yarrowia lipolytica
acgacgtacaagccaaagagatgttgactccagcagcgaattagagtaagactacagtactggtagaagtaggcctattggactgtagggagtactgcaatgaccagtgttttcggtataaatatgagaattaccgagaaaaggtaaaaaatctgaatttcggagttacgatgggtggcgttccctagtggaacatagatagacctacaattgatgggctactctgtcgcaattgtcatttctgtcactattaccacatgtgtcaagtggcagcaattgtctagtaatcatttccgttgtcgcaggccaacggctgatttgtgtaatgtggggttgagttccagcacggggcttatttggattggatgagctaatgcaagccagagtgagcatgaaaacgggcagatccccatggggaggttggccaaccccgagccaacctctttggggagagatggggaaaccctccaagaagaggagaaggatttcgtaggtcatgccgtttcccagtcagaattatttaaacgtaaaatcggttataaccatttcctggttcatgttgaaatttccacaagcttctgtatggatgtggggttctacaactttgaaattgatgacattttcgagaaaatgagaacaaccgaatatttgtactccatattgatacggaaaaattcacaatggaatcacacaagatagctctgaacaattgaggacccttttctccctaaattcattgtggttgcggttgtattgccaacatggattacgcccgcccgatgacgagcgacttgcttgagccgaggcagagtctaaatcgggacaacgaaaccataaaccccataggaagccaaagtgaccactgcatgaccccagtatataacctgccccacccctccatacttgctgctttctagatcaagttataccggacctgctaactcaattgaggtagaagatacgttattaacacaatctatttattcaacacacacacacaatgtctggagaactaagatacgacggaaaggtcgtcattgttaccggtgccggtggcggtctcggtaaggcatacgcccttttctacggctctcgaggagcctctgttgttgtcaacgatcttggtggcgacttcaagggcgacggtgcccaggctggcagtggcaagcgagttgccgatgttgtcgtcgacgagattgtttccaagggaggcaaggctgttgctaactacgactctgtcgagaacggtgacaagattgtcgagactgccgtcaaggcttttggctccgtccacattgtcatcaacaacgccggtattctccgagatatttccttcaagaagatgaccgacaaggactgggatcttgtctacaaggtccacgttttcggtgcctacaaggttacccgagctgcctggccttacttccgaaagcagaagtacggtcgagttatctctacctcttccgctgctggtctttacggaaacttcggccagaccaactactccgctgccaagctcgccctggttggtttcggtgagactctcgccaaggagggtgccaagtacaacattacttccaacgtcatcgctcctcttgctgcttcccgaatgaccgagacagtcatgcccgaggatatcctcaagctcctcaagcctgagtacgttgttcctctggtcggctacctcacccacgactctgtcaccgagtcttatggtatttacgaggtcggtgctggttacatggctaaaatccgatgggagcgaggcaacggtgctgttttcaagggcgacgacactttcaccccgtctgctattctgaagcgatgggatgaggtcacctcttttgagagccccacctaccctaacggccctgctgacttcttcaaatacgctgaggagtctgttaagcgacccgagaacccccagggacccaccgtctccttcaaggaccaggttgtcattgtcactggagccggtgctggcattggccgagcttactctcacctccttgctaagcttggtgccaaggtcgttgttaacgatttcggtaaccctcagaaggttgtcgatgaaattaaggccctcggtggtatcgccgtcgctgacaagaacaacgtcatccacggtgagaaggttgttcagaccgctatcgacgccttcggtgctgtccacgccgttgtcaacaacgctggtattctccgagacaagtctttcgccaacatggatgatgagatgtggcagctgatctttgatgtccacctcaacggtacttactccgttaccaaggccgcgtggccccacttccttaagcagaagtacggccgtgtcatcaacaccacctcaacttctggtatctacggtaacttcggccaggccaactactctgccgccaaggctggtatcctcggtttctcccgagctcttgctcgagagggtgagaagtacaacattcttgtcaacaccattgcccctaacgctggtactgccatgactgcttctgtcttcactgaggagatgctcgagctcttcaagcccgatttcatcgcacccatcaccgtcctgcttgcttccgatcaggctcccgtcaccggtgatctgtttgagactggttctgcttggatcggacagactcgatggcagcgagctggtggtaaggccttcaacaccaagaagggtgtcacccccgaaatggttcgagacagctgggctaagatcgtcgacttcgatgatggtaactccacccatcccaccactccctccgagtctactactcagattcttgagaacatcttcaacgtgcctgatgaggaggttgaggagactgctctcgttgctggtcccggtggtcccggtatcctcaacaaggagggcgaacctttcgactacacttacacttaccgagacctcattctttacaaccttggtctcggtgccaaggctaatgagctcaagtatgtcttcgagggtgatgatgacttccagaccgtgcccactttcggtgttatcccttacatgggtggcctcatcactaccaactatggcgacttcgttcctaacttcaaccctatgatgcttctccacggtgagcagtaccttgaaatccgacagtggcctattcctaccaatgctacattggagaacaaggctaaggtcatcgatgtcgttgacaagggcaaggctgccctccttgtcactgctaccaccaccacgaacaaggagactggtgaggaggttttctacaacgagtcttctctcttcatccgaggctctggtggtttcggtggtaagtctaccggtactgaccgtggcgctgccactgctgccaacaagccccctgctcgagctcctgacttcgttaaggagatcaagatccaggaggaccaggctgccatttaccgactttctggtgattacaaccctcttcacatcgaccctgcttttgctgctgttggtaactttgaccgacctattctccacggtctctgctcttttggtgtctccggtaaggctctttacgatcagtttggtcctttcaagaacgctaaggtccgatttgctggtcacgtcttccctggtgagaccctgaaggttgagggctggaaggagggcaacaaggtcattttccagaccaaggttgttgagcgaggtactaccgccatcagcaatgccgccattgagctcttccccaaggatgctaagctctaagctattatctgaccaagtgatacgagttaagtaaattttaagaaatgtatgagttaagattagttaatactgttatagtttcagttaggaccaccaccataaaaaaaaacgtagagtaatgtatcgcgctgtgtactgcaagtacagtacatactgtaccggaagtgtgagatcagtagagggtctgatgtgttaatagttcaacaaagtgttgttcaagtactgtacgaatacttgtactgtattatgtgattataaaatatatatattggatctcgccccctcccccaatataatgttattacaggtataaaaatttttatggatgaaaaaataaaataaaacaagttgcgatcaagagacttcgtggtgggacttttttattactaacaacctaaattgatcgtgcataatcgtgttgaattctcacacatcgcgccaaatcgacagctttcatgaacagagtgaatcaacaagaagcggaccagaaccctgaggctactctttatataggcaaccttgacgagaacgtcacagaagcgattctgtatgaacttatgcttcaagcgggccctatcgtgcacatcaatcttcctaaagacagagtcagccagacacaccaaggctatggcttcgtggagtacaaaacagaggctgatgcaaattacgcagcgtctattatgaaccaaatctggctgttcggaaaatccattcgagtcagcaaatctgcatccgacaagcaaaacgggtttgagattggagcaacacttttcgtgggaagtttggatcctctcgtagatgagtctacactacagcaaacgttctctgtgtttggaccatttgccaaaccccctcgaatatctcgtgacacagatggttcttcaaaagggtacggttttgtgtcttttactgattttgaacattcagaccgtgcattggaatcaatggacaaacagtatctcatgaaccagcaaataacagtcacatatgccttcaagca

SEQ ID NO: 92 - protein, 901 aa, Yarrowia lipolytica
MSGELRYDGKVVIVTGAGGGLGKAYALFYGSRGASVVVNDLGGDFKGDGAQAGSGKRVADVVVDEIVSKGGKAVANYDSVENGDKIVETAVKAFGSVHIVINNAGILRDISFKKMTDKDWDLVYKVHVFGAYKVTRAAWPYFRKQKYGRVISTSSAAGLYGNFGQTNYSAAKLALVGFGETLAKEGAKYNITSNVIAPLAASRMTETVMPEDILKLLKPEYVVPLVGYLTHDSVTESYGIYEVGAGYMAKIRWERGNGAVFKGDDTFTPSAILKRWDEVTSFESPTYPNGPADFFKYAEESVKRPENPQGPTVSFKDQVVIVTGAGAGIGRAYSHLLAKLGAKVVVNDFGNPQKVVDEIKALGGIAVADKNNVIHGEKVVQTAIDAFGAVHAVVNNAGILRDKSFANMDDEMWQLIFDVHLNGTYSVTKAAWPHFLKQKYGRVINTTSTSGIYGNFGQANYSAAKAGILGFSRALAREGEKYNILVNTIAPNAGTAMTASVFTEEMLELFKPDFIAPITVLLASDQAPVTGDLFETGSAWIGQTRWQRAGGKAFNTKKGVTPEMVRDSWAKIVDFDDGNSTHPTTPSESTTQILENIFNVPDEEVEETALVAGPGGPGILNKEGEPFDYTYTYRDLILYNLGLGAKANELKYVFEGDDDFQTVPTFGVIPYMGGLITTNYGDFVPNFNPMMLLHGEQYLEIRQWPIPTNATLENKAKVIDVVDKGKAALLVTATTTTNKETGEEVFYNESSLFIRGSGGFGGKSTGTDRGAATAANKPPARAPDFVKEIKIQEDQAAIYRLSGDYNPLHIDPAFAAVGNFDRPILHGLCSFGVSGKALYDQFGPFKNAKVRFAGHVFPGETLKVEGWKEGNKVIFQTKVVERGTTAISNAAIELFPKDAKL

SEQ ID NO: 93 - DNA, 3134 bp, Yarrowia lipolytica
tgtgttttcgtcttagcgtcatgtattctcaagcttagtcagagagaaggactatggaggagaaggggagaattgagaagggtatttgaagggactttgaaggtcgcgtggaagaggtacttgaagaggtatttgaaggtcacgtggaagaggtatttgaagatcacgtggaagaagtacttgttttacagagaatatcggggtgattttgacagtgggattgtctcccaagtcctaatcgtttgacatgggagcagtgaaaagtcgggctaaaaaagggaatatcggaaatcggaaagacggaaagaattactggactcatgtttagtagatctgagcacttcaaatttgaaaatatctcttcaaacagcagatcggttggtcgtggaggtaccatcaagggtaaaatcaaggctatcatcaagggccatatatcgcaagtttgggggaagataatatgttcatagtgaatcagggttgtggatttcctcatctaacggcattgtaactagtcctggagggtcttttttatggataacctccatgtacgatgtatccaagatctccacgtactgtgttctgtttcctaagtaatacccaacaacctctccaacaaacacttgggaagatgcacttgtgctgagatgtcaagatgttagtactgtactggatggagagaatattaataaataattgttacccaactacatcttgtcgattgaaagagatacccctaagacagataggatatctgcaacccgaggaatgaaccccccagcaccggcaccctttctattaacaaaatgccaactgaaatttgaaaagttcaactaaacttatttgacccacaaaaactcgtcaaaagtggcggcgaaagctggcaaatgatgacatccccttggaactatgatatcccctcggaatcttcgtccccatttgccacatctacttgcaacgccacgtctgcttactaagcaacccaaatctgcctcggctcaaaatgtggggaagttcacatgcattcgctggtgaatctgatctgacactacaactacacaccaggtccaacatgagcgacaatacgacaatcaaaaagccgatccgacccaaaccgatccggacggaacgcctgccttacgctggggccgcagaaatcatccgagccaaccagaaagaccactactttgagtccgtgcttgaacagcatctcgtcacgtttctgcagaaatggaagggagtacgatttatccaccagtacaaggaggagctggagacggcgtccaagtttgcatatctcggtttgtgtacgcttgtgggctccaagactctcggagaagagtacaccaatctcatgtacactatcagagaccgaacagctctaccgggggtggtgagacggtttggctacgtgctttccaacactctgtttccatacctgtttgtgcgctacatgggcaagttgcgcgccaaactgatgcgcgagtatccccatctggtggagtacgacgaagatgagcctgtgcccagcccggaaacatggaaggagcgggtcatcaagacgtttgtgaacaagtttgacaagttcacggcgctggaggggtttaccgcgatccacttggcgattttctacgtctacggctcgtactaccagctcagtaagcggatctggggcatgcgttatgtatttggacaccgactggacaagaatgagcctcgaatcggttacgagatgctcggtctgctgattttcgcccggtttgccacgtcatttgtgcagacgggaagagagtacctcggagcgctgctggaaaagagcgtggagaaagaggcaggggagaaggaagatgaaaaggaagcggttgtgccgaaaaagaagtcgtcaattccgttcattgaggatacagaaggggagacggaagacaagatcgatctggaggaccctcgacagctcaagttcattcctgaggcgtccagagcgtgcactctgtgtctgtcatacattagtgcgccggcatgtacgccatgtggacactttttctgttgggactgtatttccgaatgggtgagagagaagcccgagtgtcccttgtgtcggcagggtgtgagagagcagaacttgttgcctatcagataatgacgaggtctggatggaaggactagtcagcgagacacagagcatcagggaccagacacgaccaattcaatcgacaacactgtgctgcatagcagtgcacagaggtcctgggcatgaatatattttagcattggagatatgagtggtagagcgtatacagtattaattgtggaggtatctcgtcgcattgatagagcaatacagttactgctgaagggaatgataccgagtatttcggcccgattcagttcttgatatcgtcattttgtctctattgtctacttttcagataacctcaacaaatcttcaacaaatctcccagtaaacagtcagagatcatatccgagatcatatcagatatgtcacgatccgagtacaataatggatattaatctgcttgattttgaattctgttgcgattatgatttctttgatttcgatatgaacacatacggcgactcccagacctttagaagctccagtttggattcttagcaatggttacactcaactatatcccaagtaatacttggtaacaatatgccaagttagtcattcattcgttataggagttagcaagtgtttgtcagctaaaaatggttagtcggtcgattaccacttagatcttttcagcgtggaacttgatggtacgcttgaaccgacacttggagtagtcggggctgttgatgacgtagatgacgtttcgctcagggtgaggagtgcaatagtagtactccttggggccgtctctcagctcaaaggttccatcggcggcaatgtcaaagaccgagccctggagcttgtagccgtagtcgccggtccagaacaaagcctgcagctccagataggcgatgggcatgtcgttaacagagaaggtgttgccctcgccctcggtgatggtgatgggttcgccgtcggtggaggcggtgatcaggtcatcttggtaggtgacgggcagagattcgaccgattgggcgtctgatctggtataggtcagctt

SEQ ID NO: 94 - protein, 377 aa, Yarrowia lipolytica
MWGSSHAFAGESDLTLQLHTRSNMSDNTTIKKPIRPKPIRTERLPYAGAAEIIRANQKDHYFESVLEQHLVTFLQKWKGVRFIHQYKEELETASKFAYLGLCTLVGSKTLGEEYTNLMYTIRDRTALPGVVRRFGYVLSNTLFPYLFVRYMGKLRAKLMREYPHLVEYDEDEPVPSPETWKERVIKTFVNKFDKFTALEGFTAIHLAIFYVYGSYYQLSKRIWGMRYVFGHRLDKNEPRIGYEMLGLLIFARFATSFVQTGREYLGALLEKSVEKEAGEKEDEKEAVVPKKKSSIPFIEDTEGETEDKIDLEDPRQLKFIPEASRACTLCLSYISAPACTPCGHFFCWDCISEWVREKPECPLCRQGVREQNLLPIR

SEQ ID NO: 95 - DNA, 3917 bp, Yarrowia lipolytica
gactggctgggcagtaacggttcggtttttggtgcggaaaaattgggtccgaagttgggatgagggataaatgagaaaaatagaaatgcgtaaaaaatcccaaaaaaactgggaaaaatgggaaaatgtcggaaaacagcagaaaattgacagagagaaaatggagaattgtcagtaaaaaaatcagacaaatcacgtggccattttcgccccaattagcaccaattagcgcgctatgtctctcccctcttttacccaatatactcatttacctccgtgtaatgagactcagacaagcaaggcaaggaaagagggaagcgcttgagagatgtattgtcgtacaagtcatatgcactttgtgatgaagagcccaaaatggggattggagcgaccgtcggtttgcgccgcggtctccacgggtgccaatggcccgctggatggaccatatctcctccctcaacgacgcgttcgcgcgacggagcccactcaacgcgtataataaaagtctagatggtttggtacattccggcttgttgtgagttttcccttttctccattctgaggaaaaacacagttaaactgggttctgaggttgtatttttttaactggattttatgtatttaatttaatttaatttaatttaatttgatttaatttaattatttatgtttgagagcagcaatggggagtgttgattggagcggggctgagtcagattgagataatgcggttcgatcatcgtctccttctccaaatccaccccaatcgcactatccattgtctctctggaccaagtgactatcacttcgtacaaggcacactgtactacaagtacacttcaagagcatgtacctcccccccgagttttgtaccccttccactttttgaactttggagtggtgaaaccctaatttcgaaaccctctcacacctgccttaaatatctcacggttgctttactacattgttttctatagttactcttgcaccacctcacaccatgaaattctctccacttttgctggcgaccgtgggtctggcccagagcttctattcgcccaccgactcgtacgcgcccgggtcggtcgactgtccctccaactccacccagattgtgcgaaagggcgagggcctttcttcgcaggaacgagagtgggtccagaaccgacacgaacagacccgaccggagctgctcaactacctgaaacgggtgggctttaagtcggtggacccggaccagttcctgggccaggacactaatatcaccatcggactgtccttttctggaggaggatacagagccatgttggcgggagcaggccaatttgcggctctggacgcgcgaacacccaacgccaccgaaccgggccatgtgggaggtctggtccaggccgccacctatctggtgggtctttcgggcggaaactggatggtgggatctgtggtcatcaacaacttcaccaccattcccgacctgcaacactcgtccgacgtgtgggacctggaacactccatgatcaaccccggcggaatcaacatcttcaagaccggctcttattgggacgacatcaacgacgacgtcaacgacaagcagcacgccggatacaacacctctttcaccgacatctggggccgaggtctgtcgtaccagttcttcaacgcttccaacaccgcccgactcacctggtccgagatccagaactacgaccacttcaagaaccactctatgccctaccccattgttgtggccgacggccgagcccccggaacccgaatcatctccggaaactccaccattttcgagctggccccgttcgaggtcggctcctgggaccccaatgtctactcctttgccaagaccgagtggctgggaaccaacatgaccaacggccgacccaacggaacctgtgtgcatggctttgacaacgccggtttcattgtgggaacctcttcttccctgttcaaccagttcattctccagctcaactccaccggagtaaccggagtggtctacgacctggcccactctatcctcaagcgtctggataaggactcggacgatatcgccatctactcgcccaaccccttcaagggcatgtcctacctcggcaactcgtccattgcccaaaccgagtatctggatctggtcgacggaggagaagatggacagaatgtgccctactaccctctgctgcagcccgagagagccgtcgacgtggtcatctcctacgacaactccgccgacaccgactttaactggcccaacggaacctccgctgtccagacataccgacgacagtttgagaaccaggccaatggcaccattttcccctacgtgcccgacgtgaacacattcatcaatgagaacctgacttctcgacccgccttctttggctgtgacgtgcagaacatgacctcgctggacaagaacggttataccgacgtcaactcttccgccgcccttccccctctcattgtctacattgctaactacccctggacctttttctccaacacatcgaccatgtccaagctgtcgtacaacaagaaggaggttgccggcatgatcgagaacggctactccacctccacccagttcaacggaaccgtggaccccgactggcctgtgtgtcttggctgtgctctgctcaagcgagaggccacccgacgaaaccagtctctgggctctgactgtgacaagtgcttctccaagtactgctggaacggaaagactgacgatgatgccgaaaaggctctgaagtttgctcccgccatctatgccaacggccctaacgtttcttccaacgcctcaattggccaggcttcgtccacctccaagcccaagaagaacggcgccgagggtctggttccgggtatggctgccattgccattggttttgccgctcttcttatgtaagcattaatattgactaattgactatataacgagtttcgagcgtcagagagcgtcagcgggcgtactgccacgttccatgtgtacaacaggttgtattacaagtattggtgccgagtcagtctattattgaagctgtagatgataataccagtcattactcagcatgtggagtctgggattgttttttgcacggtttttgcgtctgacagattccaactggcgtcttttgattatttttgattatttttgattatttttgattatttttgattatttttgattatttttgatcttttttattatgatttaatttattatgatttttttaaatcttacagtcgcccattatagtgcggaaaaatgcgcaattggcacggtttgaaacatacggagcgtcgccgcgtacacaaggacgtacagatgcacggcatcagcttacaaacgctgttggcgaccgcagctccggaaatggcgctttttcgaactactttcacgcgtcaacaagttctgtgaaactcaatcgagtcgcgaagtttggaggtggatttggggaagaagaacgctggagatgagcaacggagaccgtccgacggccatcgccatcttccaccagcggtgtattcctccaacccctgaagaatcacattccttccaagctccaatgacgtaaaacggccttggctttgcggccgccgctaggagaccctgaaacctacaaacctgaactagccaaacctagatggcctaaagaggcagacaatgaaaactataatcttcggcggaatcctgcaccgaataatactttcacgcacggaagaatgactgagaaaaaatgatatccgtgagattcgaactcacgcctccgaagagaccaggaaattcgttaaggtattagccttaacctggcgccttagaccgctcggccaggataccggaagttgaaaaagaaggaaaatgggtttacacaattcgaaatatgctataaaaagtctaactaaaa

SEQ ID NO: 96 - protein, 638 aa, Yarrowia lipolytica
MKFSPLLLATVGLAQSFYSPTDSYAPGSVDCPSNSTQIVRKGEGLSSQEREWVQNRHEQTRPELLNYLKRVGFKSVDPDQFLGQDTNITIGLSFSGGGYRAMLAGAGQFAALDARTPNATEPGHVGGLVQAATYLVGLSGGNWMVGSVVINNFTTIPDLQHSSDVWDLEHSMINPGGINIFKTGSYWDDINDDVNDKQHAGYNTSFTDIWGRGLSYQFFNASNTARLTWSEIQNYDHFKNHSMPYPIVVADGRAPGTRIISGNSTIFELAPFEVGSWDPNVYSFAKTEWLGTNMTNGRPNGTCVHGFDNAGFIVGTSSSLFNQFILQLNSTGVTGVVYDLAHSILKRLDKDSDDIAIYSPNPFKGMSYLGNSSIAQTEYLDLVDGGEDGQNVPYYPLLQPERAVDVVISYDNSADTDFNWPNGTSAVQTYRRQFENQANGTIFPYVPDVNTFINENLTSRPAFFGCDVQNMTSLDKNGYTDVNSSAALPPLIVYIANYPWTFFSNTSTMSKLSYNKKEVAGMIENGYSTSTQFNGTVDPDWPVCLGCALLKREATRRNQSLGSDCDKCFSKYCWNGKTDDDAEKALKFAPAIYANGPNVSSNASIGQASSTSKPKKNGAEGLVPGMAAIAIGFAALLM

SEQ ID NO: 97 - DNA, 3740 bp, Yarrowia lipolytica
tgaagcgggaaatcaagattgagagttggagtggtgggggcggtggtggaagagacagaggtgatagaagggaaagaaacgacagaagagacgcaaaaggaagaaacagaagagatggagataggaggaatggcgatcgaagagacagagacagagatagagatagagacgacaggtcaagtcgttcaagtcggtacaaccgatcaagatcaagatctccgactgtcaaaaaggaaacagatgaatatggccgggaccggaaggactaactgcattatatataaatatagattcttgattagtgatcaaagcatgagatactgtcaaggcaagcaagccttacaatatctgttggcttagttaaatcttttttgagatgtaataattggggaacctctgttagatgaacagttgcggcttactcatcttagacatggtccagtttaggcgtaattaattatcagttttaatgcgcctctacgtaataagcgttgccacaagttacagtagagatgataccattgctacaggaccgtatagcgtatataatgacgaggctaattgatgatgatcgacagcgaagacatatctacgtcaatctgacgcttcattttgtgcttcttgttcggatttgtgtttctcccagtgcacccagttttttgctgaccattttttgcttaccagtttttgctcgcgcgttttctcgattgaactctgctgcgagggtggtgtaagacacactccctgtagaacatgcatttcgaggtgtgacatttccatctctgttaaatcaaacgagtgacacaaacggaataaatctacatatacgtcgaaccctcttcttctcttctctctcttccttaatagacaccctcgctatcgactctgctcctccttatcacaccatcaaagtttagggtgtgtcccaacaccacctccacaccacactctattatcaccacctccacgactacataccactccttccaccacctcacaaatggcgaccgaacacgtggaacacgccgaaaacgccaagcagtcgccccaaccgtccttctcgggctccgagagcggccgaattggccggtaccaaatcatcaagactctgggagagggctcctttggcaaggtcaagctcgcctaccatctggccacccacgaaaaagtagcgctcaaaatcatcaacagaaagacgctggccaagtcggatatgcagggccgagtcgagcgggaaatctcgtacctgcgactgctcagacacccccacatcatcaagctgtacgacgtgatcaagtccaaggatgaaatcatcatggtgattgaatttgccggcaaggagctctttgactacattgtgcaacgtggaaagatgcccgaagacgaggctaggcgattcttccaacaaatcatctccgcagtcgagtactgccatagacacaagattgtgcacagagacctcaagcccgagaacctgttgctcgacgaaaacctcaacgtcaagattgccgactttggactctccaacattatgaccgatggtaacttcctcaagaccagttgtggctctcccaactatgctgctcccgaggttatcagtggcaagctgtatgctggccccgaggtcgacgtgtggtcctgtggtgtcattctatacgtcatgctgtgcggccgtctcccctttgacgacgaattcatccccaacctcttcaagaagatctccaatggtgtctacaccattcccccctatctgtctgccggcgcaaaacacctgcttcaacagatgctggtggtcaaccccctgaaccgaatcaccgtccagggcatcatggaagacccctggtttaaacagggcattgccgagtaccttgttccacgtgacctgaagcctgatcaggtggaaatcgacgacaaggttgtgggtgctctttcagacaccatgggctacgaccgagaccaggtctacgaggctctcaaggctcctaaatccgacggaaccgaaatccgagatgcctacgacctcatgaaggagaacagacagcagttggagaaggactcgaccgtgttcgacaaggtggacgaatccggcaagcctaagcaccagttctcgcgaaagaacccgccggctctgacgtttgactcacatcacatgcccaccattgtcgaccattctgatcttcgaacccctaacacaaccattgctgttcttccctcctcgctgccagcataccatcgggccaacatgatggcccatggtcccgctaccctgcgtaagctgaatcccatctcatcgcgaaaatcaaagacccgatggcatttcggcattcggtccaagtcgtaccctctcgacgtgatgggtgagctctaccgagctctcaagaacctgggagccgagtgggcaaagccatcagagaacgacctgtggaccatcagggtgcgatggagatacacccctgaaaaggatgagtcggacagcacaactaagccagacagcggactgcttaagatgcagattcaactgtatctgctggagcagaactcgtacttggtggacttcaagtttgacggctgggagtgcgagacggagagcggtgagattaagacccgcgatgtcgcccaggtgtcgaattcaaagtccgccaacgagaccttcacgttttccgcataccccttcttgcatctggccacccggctcattatggagctggccgtgagttctcagaaggagagtgagaagtaagcacttgtagagcacactagggatttagaggggattatggcacgtacaatatagataattaagcagtagctgagtcagttgaatgatcagaggtgtaacatgagtgtggatggattgtgtagagtcgttgtttaaaataatgagttaagaataattatacgactacaggatacgatgtacttgtattgtatcgatacagtacatacagtacatacgtgtaacatactcctaaactgttgcatcacctacaactccaactagtcggtcataattcattaatacgtttccctggtgtagtctaggccaaacactccttggtgaacttcttgagaaagattcgctctctagactccttgaattctctaggctggatttcggcccaaaaagaacccacaaacggcgcaatactcttcttgtagccggaatagttgtctcgaatgacaatatccgggttggcagaccgctgctgatgctccttgacgatgatactctcaaagtggtccacggtagacatgacaataagagccgacgaggttttgctcttgtagttggcgatgagcgttttaagtgccccagagttctccaccaccttgtggatcagctgatgcttttgttcctcgctgagtccttccgaggccagggggatagccaagcaggtgagaatagcgcctgtgtctcgtttagaagaggtgctgtttgtatcatccgcatgcttgttgatgaactgaactaaattcagtagcgtgctccacagctcattccagtggtaatttgagggagtgtccctgtaaaccatgagtgttgcttcagtgacgaccagagcgagatcataaatgtctgggagaggtttcttcatattgtatctgagacagcaaagaagtgcgtccaaaaggccggttccaaaggtgtaggcctctgtttctggaagggggggtttccgttgcttggcgttgattttaatagatgcttttgtgttgaacgactggtgcaattcgggcag

SEQ ID NO: 98 - protein, 579 aa, Yarrowia lipolytica
MATEHVEHAENAKQSPQPSFSGSESGRIGRYQIIKTLGEGSFGKVKLAYHLATHEKVALKIINRKTLAKSDMQGRVEREISYLRLLRHPHIIKLYDVIKSKDEIIMVIEFAGKELFDYIVQRGKMPEDEARRFFQQIISAVEYCHRHKIVHRDLKPENLLLDENLNVKIADFGLSNIMTDGNFLKTSCGSPNYAAPEVISGKLYAGPEVDVWSCGVILYVMLCGRLPFDDEFIPNLFKKISNGVYTIPPYLSAGAKHLLQQMLVVNPLNRITVQGIMEDPWFKQGIAEYLVPRDLKPDQVEIDDKVVGALSDTMGYDRDQVYEALKAPKSDGTEIRDAYDLMKENRQQLEKDSTVFDKVDESGKPKHQFSRKNPPALTFDSHHMPTIVDHSDLRTPNTTIAVLPSSLPAYHRANMMAHGPATLRKLNPISSRKSKTRWHFGIRSKSYPLDVMGELYRALKNLGAEWAKPSENDLWTIRVRWRYTPEKDESDSTTKPDSGLLKMQIQLYLLEQNSYLVDFKFDGWECETESGEIKTRDVAQVSNSKSANETFTFSAYPFLHLATRLIMELAVSSQKESEK

SEQ ID NO: 99 - DNA, 7490 bp, Yarrowia lipolytica
gcgtccaaaaaattcaaatttcgacatttttcggcttaaaatagaacatctacaagacgatgttgcagactcctgagtctgggaaaaggtggttgaggaacctacactggagctgcgattggagagcttcgtggcggccgtgggccaagattgggcagagtatgggtcgagcatttctattgcttctactgtacttgtacttgtacttgtacttacttacttgtacaagtacagacaggaggcttgctatttcgatgaaactaaagcacgtttgttatgtaccgaagttcggaacgaggctgtggagatggcaagtagttaagtgtcattaattggagtaggttaaaacatcaactgtgctcgttctaactgcttactgcgcctgttataccaccgctagcgccctctaacggatcctaggtgcgggttgaactggtttatgattgctgtttttacactttttagacccccagagcgtgcaggtgatggagtgaacgcgcggtgctacagtaatactctaagacaagtttgggcagggaaggaaacacctacaggaggccaggtggccaagatttagacggtttggggagttgtatttgttgccacgtacaaagggacaaaaacacgggcttgtagtggccgcaattccactgctcttgtgcttgtcagctgctactcttcgtctaagaggaccccttcttaatgtgaactcagctgcatgaccgcgcatacttctttcctactgcagcccctgacaggcaatactagcaagtgaagcatccattctacgacagaggtattttcaggacgaaaaaacactcttgagtcgccacaaaaagtgagtgttccgacaacgcgggcggagcagtttggagtttgggagcagtgtattgatctgactgagcttgagagagtgtgtgacattctggagggagaagggaaaggggagatgatatgatggatatgggttggggagatgatggtataggactggttgtatatggaaacgatgcttgatcgtgttattccactcagctgtgtacctccttacgccacaccctacagtcacgttttcaccacccccatctcaaacaacactaacacagtgttctacgaagacaccgccgactccgattggcagtcgtccgactacgacagtgaacatgcgaccgtcaacgtgcccatccggagcaaacggaaaaagaatgctgctaaggagggcaatccagtggatcccatctctgaggatccgcaccaggaatccttttcagatggaagtgaagctgcgggacccagcgctcccccggccgacggatacatcaacaccgcgcccaatggcgtctactcggaggaagatcttcctggaacccgccaggtgcttccaggacctcaggaggtacccgacgtgacctccactccccagatgaaccgccatcccacagcagatgaatctactcctcagggctcacggtggagccgaggcagagagggatttggacatcttggaagtccctcgttctttagacgacacagtgccacagctgaagatatgacccctggaggaggacctcgacggctgttcaagcgaccacagctcaactcttccaacaccacatcgggccagcgttggagagatatcaaagccgggcttcgtcagcggttccgaaagaagaaggacgcgaaattcagcgctgagaaggcatacaatcaagccattctgggccagcttgctgctggttctcctgcggctctcatggttgcttcctccatggttcgcgacgaaaagaacatgaagcgagtgcccattctgctggagcaggtccgtttcgctctcagagatgtgacaaagaaccctcacgagaagaatcgacagtatcgaatcgatctttcgtacggttccggtcacacgatgctcgcatggacaatctacaaggactacaaggactttatgtttctgcacagtcggctgcgaaccatggccttcaacgccaagaaccccttcaactcgtcaaaacagcctcttcagatccccatctttcctacccgaaacaaggtgggcaagaaaaaaaaaggcaagagagacaagtccaaccctggcgatgaagattacacagacacagatggagagtctgagtgtgatcccgcagagcagcaacaaaaccagcacactccaggagggggatcttcgcatggacctaggatggtgaaccagttctttgacaagcgacggaaaaaccgcaacatgatcaacgagcgcattaagggtgaactggaacgatacctgcgtcagctgttcaaactgctgctcttccgaggtgaagctaacaagctgttccagtttttggagctgtccaacatgtccattcgacttgctcctgaggaccatttccacggcaaggctggattcctggtcaaccgtacgtctgctcgaaaggctggatggagagtgtcgcattggcgagtcgacgatatcagacagatggtggctcgtcacacgtcgaagtggttcatggtgcgccactcgtacattgtgtgtgtcgacaacctgtattccacaacacctcttgaggtgttccttctcgattccaagttcaaggtgtcacacaatctcaaaaagaagaacaaggacggcgttctttcgggtggatccaaccccaatacttctggagagtccgatttggaagaacttcagaaaccgggaagtacccatattactctcaaggtggagaacgcatcccgacagcttaagctggttgccacatctgacaagcagctggcccagtggatggagagtatcaatctgatcaaggagcggtccatttgggctcagcagaagcggtttgactcatttgcgcctgtgcgaaccaactgcaaggcccaatggttcgtcgacgctcgagactacttttggacgctgtcgtgcgctctggatatggccaaggaggtcatttacattcacgactggtggctgtctcctgagatatacctacgtcgaccccctgagggtaaccaggagtggcgtctggatcgtgttttgaagcgaaaggccgagcagggagtcaagatttttgttattgtgtaccgaaatgtcggtcagactattcccattgactcgcagtacaccaagttctcgctgctggacctgtctcctaacatttatgtcatgcggtctcccaaccagctcattcagaatacctacttttgggcccatcacgaaaagctgtgtcttattgatcacacgtgtgcctttgtgggtggtattgatctctgttttggtcgttacgatactgcggaacacgttctcgtcgacgatgcgcccacgtttgtgaatgccgacaagaaggacggctacaccggtcgaactcagctcttccctggtaaggattactctaacccccgtaccaaggacttcttctcgttggacaagccttttgaagatatgtacgaccgtcagaaggtgccccgcatgccctggcacgatatccacatgatggtagtaggccaacctgcacgtgatctggtgcgtcatttcgtgcagcgatggaactatgttcttcgacagaagcgtccttcccgattcacgcctcttctgctgcctcctccagacttcaaggaggaggagctggccgagtacaagctgaatggtacgtgtgaggttcagattctgcggtctgcttgttcctggaacactggagtcaaggagcacgagcagagtatccagaatgcgtacatcaagagcattgagcagagtgagcatttcgtctacattgagaaccagttttttatcactcacacctcgtggcacaatattgtgattgaaaacaagatcggagacgctctggtcaaccgaatcatcaaggcccatcagaatgacgaggattggcgtgccatcattgtcattcctctcatgcctggttttgaagccgaggtcgacgagtccgagggttcgtcggtgcgtgtcatcatgcagtgccagtacatgtccatttctcgaggtgccaactgcatttttgcacgtcttgagaacgctggcatccatcccgaagactacatttgcttcttttcactgcgaaagtggggcaagatcggtccccacgacaagctggtcaccgagcagctgtacatccacgccaaggctatggttgtcgatgaccgtattgccattattggttcggcgaacatcaacgaacggtctcaacgaggtactcgagactctgaggtcgcagctattgttcgagacacccatactgtggactctgtcatggctgggcgtccctacaaggtgggccactttgcacacaccctgcgtctccatctgatgcgagagcacatgggcgttgatgtggaccatgtggagttcattgagcgaaaggctgagcttcttgagaaacacaagagagctgaggctacccaagatgagccccaggagcttcctatcatcaagaacaagtccacacgccgagcacgggggcgtggacgacgttcccagcgaggaggagaggattccgatggagcttccggctctgaagatgagcgagttctcgatatctcaacctctatcttcggggagacggttcaggctgccaaggccgctaacggtaatggcaatggcaacggtaacggtgctgatgcgtctgctgcggagtctgtcccttccgtcatggctcccacccgaagatactcttaccaggatggagtacctgcctctccttctattgacgaggactgtgttgtcgaaggcacaactctggacaatgctgaccagcagctagccaacttggacgatgcgtacgattccgacgccgaggtgctactgcgagactatgatgaggatgtggatcagatcaaccggggcttggacatctacaccttcaactctctagccggtgtggacaacaagggcatccgagagaagaagcttgtctcttctgataaccgtatccagggcaacgagaagcaccgtgctgacgttgagggccggggtctggaccgaggcgccaactcgaaggagtctgcggaggacttccgaatccgaatccagcgacacgcccagtttgccaacaagctgacggagcacgagctgcagcagaccagcgagacggtggacgatgtgacggaacttaagaagcgcgtctactacgagcagatggagctcaagcagcggtaccacaattcgcacatggaggccgagaaggcgttcaacaatcccccacagttggaggactgcgagaacgagaagaacggcgttaatggagtcaacggcaacagtggctccactgattccactgcacctgtaaatcttgtctccgatgctaacggcaacattcaccctgaacatcctgagacatctgatcattcttcggactcttttgaagatgagggcttcgagatccccaagatccctcctcccgtagacccctactgttttaaggaccctttggacgacgacttttactacgacatctggctggccacagccgaaaagaacaccaggctcttccgggaagtgtttcgatgccagcctgacgacgaggtgaccacctggagagattacaaggatttcaccacttacgccgagcggttctccatgtctcaggacaccggcgacttgaagtcttcgtatgcagatgaacaccaagctcattcccccttggctgtcaagatcaatggcaatgacaaggaagacgagaagaaacagaacaagcacaaggccacacatgactacgacaagtattatgcccgtcaccagcaccaccgatcgtcttcgcctcactaccgaaggtcccatgtgcctctcaagaccggtgccaacttcactgatcgacttgcacacggtgttgatgatgccgctggtcatctcaagtacgccctcggacgggccaaccgccgaggcagccaccatgacactgttgatgtggatgattctcctcccagggacttccttcctgacgatttcaacgacccgatccctgtcgaggaggtggacaacaccaacctcaacaacaaccagcagctcaaccaggagctttacgagtcgcaggtggagggcgagaaccctcgatttgtgcctagccacgccattgatggccagcaccctcgaaagcgtgagaagcgacacattggtgaggttgatcagcctgccgagaagccccccatcaacgaggcttacagcactggggtctcaggccggcttgggcagcaccagcagccccatcttaagcaggaagttgatactgattttaaggatacctcttctgctgctcatacgccattccaccatacccccaacgccaggcgaagacgacgacgacgggctcgacagagatctggcatggacaaggtgcacgacaaggcaacggccgagaagattctcaacggtatccaggggcatctggtcatgttccccacccactggctgtacaaggaactcgacagcggcaactggttctacaacctggatcgaatccccccgatcgaaatttatgattagaatggtaatatattggtatgataaataatgcaagcacttgcatgagtgtactgtacgtaagcaagtagtgacagttcagttaatcaaatgcaaacccgctgcgacaaacacgtgctaagctcttcagattcttcatcactggtctcgaaatcaaagagctcacaggtgtcttcttcctctttctggtcactggcaaagttccagtattgacgacggacgattcccgaaagcgtccagatttccacctgttgtttgatcttgtcgaagaagattagtgcaatgatgaccgaggccagctcgggaatgatgtacgggagtcgagacatgatctcgtctggttttaggggagtcttgtcgtcttcggggagatctgtgaaggtggtgtacagaaacgtgaatgcaatatggatctgaagaatcttttgtgtttccagagcattgtgaacgattttcacgatggtggcgtcgtaggcagccttcttatgagcgtcaggaggtccattcagcccggtggagaaagagtccacgatcacgtatctgattttcgagctgttgtttgacttttcaatgtgtccgatgattgtgtctgattgcagctcggtggcgagctggtcgaaggtggttttcacaatcacgaggttctggcgaatataactggcaagcacattttcacccagaccagtggattgatccagcacaaagtagttgatgacgttgtttttcgtccatgagctgtcgagatccagaaccagaaccgtcttgttctcgcctcctggagtgtcgaatggcaaacacacacggcacagctcgtacagcgccgtggttttgcctgatcttggagctccggtcagagtgatcagccctgcaagctgtgctgatggcaaatgcagccagtgtaacaagttttctgccgcatcagacggaccgtaatgctgaatggcgtcgaacaagttttccttcttgttttccgtttccatgatggtagttgtcagtgatgttc

SEQ ID NO: 100 - protein, 1829 aa, Yarrowia lipolytica
METMLDRVIPLSCVPPYATPYSHVFTTPISNNTNTVFYEDTADSDWQSSDYDSEHATVNVPIRSKRKKNAAKEGNPVDPISEDPHQESFSDGSEAAGPSAPPADGYINTAPNGVYSEEDLPGTRQVLPGPQEVPDVTSTPQMNRHPTADESTPQGSRWSRGREGFGHLGSPSFFRRHSATAEDMTPGGGPRRLFKRPQLNSSNTTSGQRWRDIKAGLRQRFRKKKDAKFSAEKAYNQAILGQLAAGSPAALMVASSMVRDEKNMKRVPILLEQVRFALRDVTKNPHEKNRQYRIDLSYGSGHTMLAWTIYKDYKDFMFLHSRLRTMAFNAKNPFNSSKQPLQIPIFPTRNKVGKKKKGKRDKSNPGDEDYTDTDGESECDPAEQQQNQHTPGGGSSHGPRMVNQFFDKRRKNRNMINERIKGELERYLRQLFKLLLFRGEANKLFQFLELSNMSIRLAPEDHFHGKAGFLVNRTSARKAGWRVSHWRVDDIRQMVARHTSKWFMVRHSYIVCVDNLYSTTPLEVFLLDSKFKVSHNLKKKNKDGVLSGGSNPNTSGESDLEELQKPGSTHITLKVENASRQLKLVATSDKQLAQWMESINLIKERSIWAQQKRFDSFAPVRTNCKAQWFVDARDYFWTLSCALDMAKEVIYIHDWWLSPEIYLRRPPEGNQEWRLDRVLKRKAEQGVKIFVIVYRNVGQTIPIDSQYTKFSLLDLSPNIYVMRSPNQLIQNTYFWAHHEKLCLIDHTCAFVGGIDLCFGRYDTAEHVLVDDAPTFVNADKKDGYTGRTQLFPGKDYSNPRTKDFFSLDKPFEDMYDRQKVPRMPWHDIHMMVVGQPARDLVRHFVQRWNYVLRQKRPSRFTPLLLPPPDFKEEELAEYKLNGTCEVQILRSACSWNTGVKEHEQSIQNAYIKSIEQSEHFVYIENQFFITHTSWHNIVIENKIGDALVNRIIKAHQNDEDWRAIIVIPLMPGFEAEVDESEGSSVRVIMQCQYMSISRGANCIFARLENAGIHPEDYICFFSLRKWGKIGPHDKLVTEQLYIHAKAMVVDDRIAIIGSANINERSQRGTRDSEVAAIVRDTHTVDSVMAGRPYKVGHFAHTLRLHLMREHMGVDVDHVEFIERKAELLEKHKRAEATQDEPQELPIIKNKSTRRARGRGRRSQRGGEDSDGASGSEDERVLDISTSIFGETVQAAKAANGNGNGNGNGADASAAESVPSVMAPTRRYSYQDGVPASPSIDEDCVVEGTTLDNADQQLANLDDAYDSDAEVLLRDYDEDVDQINRGLDIYTFNSLAGVDNKGIREKKLVSSDNRIQGNEKHRADVEGRGLDRGANSKESAEDFRIRIQRHAQFANKLTEHELQQTSETVDDVTELKKRVYYEQMELKQRYHNSHMEAEKAFNNPPQLEDCENEKNGVNGVNGNSGSTDSTAPVNLVSDANGNIHPEHPETSDHSSDSFEDEGFEIPKIPPPVDPYCFKDPLDDDFYYDIWLATAEKNTRLFREVFRCQPDDEVTTWRDYKDFTTYAERFSMSQDTGDLKSSYADEHQAHSPLAVKINGNDKEDEKKQNKHKATHDYDKYYARHQHHRSSSPHYRRSHVPLKTGANFTDRLAHGVDDAAGHLKYALGRANRRGSHHDTVDVDDSPPRDFLPDDFNDPIPVEEVDNTNLNNNQQLNQELYESQVEGENPRFVPSHAIDGQHPRKREKRHIGEVDQPAEKPPINEAYSTGVSGRLGQHQQPHLKQEVDTDFKDTSSAAHTPFHHTPNARRRRRRRARQRSGMDKVHDKATAEKILNGIQGHLVMFPTHWLYKELDSGNWFYNLDRIPPIEIYD

SEQ ID NO: 101 - DNA, 3863 bp, Yarrowia lipolytica
aggtgcccattcagattcagcccgggtaccagcccttgaagggtctttacagtgacgtgagtatgataacagaaggaaacgacgaccggtgaggaaagtgatggtatttgtggtgggagccatttgtcattggcggttggtcattggtcattggtcattggtcattggtgattagcgattggtgattagcgattggtgattggtgattggtatggtctgattactgaatggacttgagacacgaagcagatggaccatgataaaccatgatacagctcttgacttacttgatttgaacgtgtacttgagaaacagttttattgttttcgagctattcaactgcttggtctgagtctgtttggtgtgtgagctcacaccgtttggtatgtttggtatcactatgtcaatgcttctgccaatgtcatgtagtctccagaatgaggcacttgttttccttctctgtcgtttgtggcacctccttatgtgtctcggttcttgtggcacgtttttttcttttcgttctatctcggttccctcgtcatctcatgatactcaccgttgtcaggctggcagctcgcacttgcaccatgtttcggtctttgctttcccggctacgttccctgcccacacatatatctgtttctacgtgcatgtgattcccccacatgtgcctgtgatatctatgtaccgtggctaggattatgcctgtgattgtgtcatgataaccattctgcgcagtgtgattgacgatcagggtcttggtgggtacaaactagctttttgggtttctttttttcgctccaaaatgtgagcgtgaaaatgcacgtgagctaaaagagtcccaacacccaccaactgcgctggctcgactgttgagcatgttgagtgagaggagaggagaaacaaggcgcggaaaaaagggacgagaaaagaaccatagggccaaagacaaaaaaccacagacacatgtcacatgcccggattatgccccgatcagtgcatgcggaagaccgacccggcgatagaaggagaagaaatacgaggtgcagaagtgtcatcctccgccgtgattgcattttgcgtggtaataaccacgagccacgcactttgtccacgcacatgtctatggtgtaattgtacctttcctcccaacgcacccaacccaaacctaaaccctaagttgcacaaaacacatgaacgtgatactgcaaacttcccctctgataaaggcgctgtgtccatatcatctcagcacaaccacatctccaacaccaacacgacttttgccacctcgggagcccagagccagtaccaccggctctccaacaacaccaagaccttcactcgagactccccctaccattcgcgaaccccttcagtggatgacgacggaataagacaagcatcgtttctcaaccgagtctaccagtcctccaagtcctacagtccccgattccgctacggagcagagatagtcgaacggtgggcaagttctgaaaacaccaacgcagcaacaccttcggcgtcaaccacaaacagcaacaacagcaaccacagcaacacgcctgtttcaaccacaccctcaagcacaaccatgcccaaggctctgtcttcaaagtcgctggacgccgccagcatccacatggcatcgaacggtgcgcctccactgatacaaaagtcgtcgttgcacgactttgagcgccttaaatcgggaattgacgacattcaaaactcagacaccaacggtacccagtacagtgtcgatgtcggctcccagggcctgcgcatgcgtatccagacccaagggtacgcccccagcggcaacagcaacagaggcagccccgtgccaccgtcaccggcgctgtccacgtcgacctcctcgggctccacttctgctgcagcctccagatccacctcgtctcctgtgccacaacaacctcagacagctgccacagctgcaggaggacctcgacccggacaacctcggtcggcatggcaggaggtgctgatcagcgccacgtcgctggcatcattgtcgcaggactcgcgaaagcgactgcgatactgtctgcatctccttaagctcgccaacgcccatctggcttccaccgtcacaaagctgcaaggcgccattgccgaggagactgcatacagtctcgcgcagtccattgccgccaaccatgtgcctcacaacgagcgacaggcgtacctgcatcagcctccctccgctgagcctgctctctccatcaccgctctcaaggccgatgtcgtgtccacgatccgcaagatcatcaaggttgtcagccagtacgccggcaacagtctacccgagccggctcggtcgcacatccggacatacattcttggcctgccctctcggtgggcctctaccaccgcttccactaacatcaccccaacccggtctcctgccggttcccagtctcctgtgggctctcctaaggagtgtgtcacacctgaccaccagggtcctcctcttcccacaaacacagtgtcgtctccagtgcccgagggcccttctgacgagcagttgtcgcggcatcgaatcgaggttgaggctggaggcaaggtgttgattcttgccaacgaggctctcgatatgctgggcaacatcatcagtattgtggatggcaccctggagcgtgccgagggttggtgcgagggcatcaaccgggtcaagcagagagttgggctcggtgaaaccgctcctggcgcagctggggaacctggagcctccaccgaggcttctgctgaggcttctgctgaggcttctgccagtgccgccgctgcgtactctgctgttgatacctccacggaccaggacgttgagatgggagatgcctaaggctggcatgacacgacaagggacacagatgaaatgcatgatggatgatgacacaacacacacagcatgaagcatggcatgatggagaacgacatacagcacaacacaacagagagaggatagcataggcacaacatatttcatgtactcaccacacacagaacaagcgacactgacacatacacacacacacacacacacgcacacacagcagcagcagcagcagcggcagcagcagtacagcattatgcaacccctcacagatgatgcaacacttaaatggttacacacatggcatatttaatcacctctcttgtgggtgctccagaagatggcttttggcgtcggatgacggcactggttttcattttcctcgattacatgtctgtctcgggcgttgtttgtcccctcggcactttttcggcctcggctacccggtcctcgggaaagtgtgtcaaggttcacaacggtaaaaaaagtcaaaggtggtggcgttttgggaggttcggtttgaagagcggagctactgtgggcagcagcgttaaacttggtgacaagacaacggctggacgctgacgtttaccattaattggcgctgctgccgtgggtggctactggatttggtcttttgcacgaagacttatgcgttgctgcttgcgagtgtgtctctgtcttggggtggtttgccacgtgacgggccggtgtgacgccgagcggaattcgtgtttcttgccccttttatttccgacatgttccggctgctgcttctccagcatgttaccccacaaccgacgggtaaaatttctggacgcgcacaaaccttttttggcttgcggtccaggggaaaggaaccgacagatgtctaccaccaccatcaacaagagcacaccaccagtaccaggagtacactgttaccgccattagcaagtagagtttatttatataatgcaaagattatgatgggttttgggccgtggtgttggtgttggtgtctgtagaa

SEQ ID NO: 102 - protein, 620 aa, Yarrowia lipolytica
MRKTDPAIEGEEIRGAEVSSSAVIAFCVVITTSHALCPRTCLWCNCTFPPNAPNPNLNPKLHKTHERDTANFPSDKGAVSISSQHNHISNTNTTFATSGAQSQYHRLSNNTKTFTRDSPYHSRTPSVDDDGIRQASFLNRVYQSSKSYSPRFRYGAEIVERWASSENTNAATPSASTTNSNNSNHSNTPVSTTPSSTTMPKALSSKSLDAASIHMASNGAPPLIQKSSLHDFERLKSGIDDIQNSDTNGTQYSVDVGSQGLRMRIQTQGYAPSGNSNRGSPVPPSPALSTSTSSGSTSAAASRSTSSPVPQQPQTAATAAGGPRPGQPRSAWQEVLISATSLASLSQDSRKRLRYCLHLLKLANAHLASTVTKLQGAIAEETAYSLAQSIAANHVPHNERQAYLHQPPSAEPALSITALKADVVSTIRKIIKVVSQYAGNSLPEPARSHIRTYILGLPSRWASTTASTNITPTRSPAGSQSPVGSPKECVTPDHQGPPLPTNTVSSPVPEGPSDEQLSRHRIEVEAGGKVLILANEALDMLGNIISIVDGTLERAEGWCEGINRVKQRVGLGETAPGAAGEPGASTEASAEASAEASASAAAAYSAVDTSTDQDVEMGDA

SEQ ID NO: 103 - DNA, 178 bp, Mortierella alpina
  strain ATCC 32222
cataatcaaagtgtttttatggcacttttaaaaaaatccatatccaccttgtgtgcaatgtcatctcactggaggccggcggctgtaaaaagcccgtttggtcacctttgggatttatactactcagaactttagtgattttgtctgaaaaatattatgaataacttaattcaaaata

SEQ ID NO: 104 - DNA, 640 bp, Mucor hiemalis
  strain 14183 isolate 1
tctggtagtgactgcggagacattatatgatttatatggtctccgtagaacttgttctaattaggttcattcttttttacagtgaactgtttcaattttcgatgtttggggactgtcttttaggaataaggatagactattaaaatgttaaccgaactgaagtcaggtttagacctggtatcctattaattatttaccaaatgaatttcctattattattgaaagataagcgtgaaaaccttgggatccaacttttaacaacggatctcttgtttctcgcatcgattaataacgtatttaagcgccataactagtgtgaattgcatattcattgaatcatctagtctttgaaaacaacttgcgctcaatggtattccattgagcacgcctgtttcagtatcaaaaacaccccacattcataattttgttgtgaatggaactgaaaatttccacttgtttgaattctttaaacttattacgcctgaactattgttctttctgcctgaacatttttttaatataaaggaatgctctagtattaaaactatctctggggcctcccaaataaaactttcttaaatttgatctgaaatcaggcgggattacccgctgaacttaagcatatcataaaaaggggaaa

SEQ ID NO: 105 - DNA, 669 bp, Mortierella alpina
  strain 14183 isolate 2; designated strain yNI0133
gcgtaaggttactgcggatgacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatatctaaaagggggggaaa

SEQ ID NO: 106 - DNA, 671 bp, Mortierella alpina
  strain 14183 isolate 3; designated strain yNI0134
ttcgtagtgactgcggaagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatatcaaaaacggggggagaaaa

SEQ ID NO: 107 - DNA, 672 bp, Mortierella alpina
  strain 14183 isolate 4; designated strain yNI0135
gtcgtcagtgactgcggaagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatactaaaaggggggggaaatag

SEQ ID NO: 108 - DNA, 668 bp, Mortierella alpina
  strain 14183 isolate 21
gtcgtagttactgcggaagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatataaaaatgggggggaaa

SEQ ID NO: 109 - DNA, 671 bp, Mortierella alpina
  strain 14183 isolate 22
ttcgtcagttacctgcggaagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatatctaaagcgggggggaaa

SEQ ID NO: 110 - DNA, 670 bp, Mortierella alpina
  strain 14183 isolate 23
ttcgatagtgacctgcggaagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatacaaaagggggggggaaa

SEQ ID NO: 111 - DNA, 824 bp, Trichoderma asperellum
ttcggtaggtgactgcggagacattaagaatttaataattttgaagatggatttttttttttttggtttgggcaaaagcaggaaaccttttaggggggaaaaaaacaaaaaagggaaagcccccccgggccgggccccaaggggggggcctggccgggctggtgagtttcttcctgcctttcccaaagggggaaaaattcctgggttttggttaaggaacaataaaaaccgttcacccccaccccccggggggttttcttaaccttggcactttttccttggggcttccaacaaacggggccccaaaggtcaccaaaccccaccatttttttctttcctttaatttttggcaaaaaacaaaatttttccaaccgggaatttttaaaaattaaaaaattttcaccaccggtcctttgggtccccccaccaagaaaaaacccaccaaaaggcaaaccgaaggggaaatggcaaaatcccgggaaccaccaaacctttaaccccccttggcccccctgggtttcccggggggcagcccggttaaacccctttttcctccccaaactttcggttgggaaggggggaaccctcttgggagtaaactggaaatggcgggcttttccattggagttttttttttcccaaaaagggtttcccctgggggttggaggaaaaggccagacggccttttagttttcccccgggggtaatctttttatccggggggatggaacgtatcaaaaaaaaaagccccaggcaaaaatggtcttaaatttgccctcaatcgggaggaacccccctgacttagcaatcaaaaagggggaaaa

SEQ ID NO: 112 - DNA, 668 bp, Mortierella alpina
  strain 14183 isolate 25
tcgatagtgactgcggagacattcataatcaagtgtttttatggcactttcaaaaatccatatccaccttgtgtgcaatgtcatctctctgggggctgccggctgtcaaaagccgtgtggtcacctttgggatttatatctactcagaactttagtgattttgtctgaaacatattatgaatacttaattcaaaatacaactttcaacaacggatctcttggctctcgcatcgatgaagaacgcagcgaaatgcgatacgtaatgtgaattgcagaattcagtgaatcatcgaatctttgaacgcatattgcgctctctggtattccggagagcatgcttgtttgagtatcagtaaacacctcaacttccatatcttttgtgaaatgggagttggacttgagtgatcccaacgcttttcctcaccgaaaagtggcgggttacttgaaatgcaggtgcagctggacttttctctgagctataagcatatctatttagtctgcctaaaaaacagattattacctttgctgcagctaacataaaggagattagttcttgtgctgactgatgcaggattcacaaagacaggcttcggccgactttgtaaactcgatctcaaatcaagtaagactacccgctgaacttaagcatatctaaaagggggggaaaa

SEQ ID NO: 113 - DNA, 644 bp, Mucor hiemalis
  Namadji I isolate 1
tctggtagtgactgcggagacattaataatttagatggcctttgctagttttctagcgaatggttcattcttttttactgtgaactgttttaatttttcagcgtctgaggaatgtcttttagccatagggataggctactagaatgttaaccgagctgaaagtcaggcttaggcctggtatcctattaattatttaccaaaagaattcagtattataattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaattgagagtttcggctttattgctgaattctttaaaattattaggcctgaactattgttctttctgcctgaacatttttttaatataaaggaatgctctagtaaaaagactatctctggggcctcccaaataaatcattcttaaatttgatctgaaatcaggcgggattacccgctgaacttaagcattcaaaaaggggggggaaa

SEQ ID NO: 114 - DNA, 639 bp, Mucor hiemalis
  Namadji I isolate 3
cttaagtgactgcggaagacatttaataatttagatggcctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaactgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatactaaaatggggggaaaa

SEQ ID NO: 115 - DNA, 647 bp, Mucor hiemalis
  Namadji I isolate 4
ttcggaaagtgaactgcggaagacattaaataatttagatggcctccctagaacttgttctagttaggttcatttcttttttactgtgaactgttttaatttttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaactgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatactaaaagggggggggaaa

SEQ ID NO: 116 - DNA, 639 bp, Mucor hiemalis
  Namadji I isolate 5
tcctgtaagtgactgcggagacattaataatttagatggcctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaactgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatcaaaagccgggggaa

SEQ ID NO: 117 - DNA, 640 bp, Mucor hiemalis
  Namadji I isolate 6
tcgttagtgactgcggacgacattaataatttagatgacctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaattgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatctaaaaggggggggaaa

SEQ ID NO: 118 - DNA, 639 bp, Mucor hiemalis
  Namadji I isolate 8
tccgtagtgactgcggagacattaataatttagatggcctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaattgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatctaaaagggggggggaa

SEQ ID NO: 119 - DNA, 646 bp, Mucor hiemalis
  Namadji I isolate 9
cttaagtgactgcggaagacattaataatttagatggcctttgctagttttctagcgaatggttcattcttttttactgtgaactgttttaatttttcagcgtctgaggaatgtcttttagccatagggataggctactagaatgttaaccgagctgaaagtcaggcttaggcctggtatcctattaattatttaccaaaagaattcagtattataattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaaatgagagtttcggctttattgctgaattctttaaaattattaggcctgaactattgttctttctgcctgaacatttttttaatataaaggaatgctctagtaaaaagactatctctggggcctcccaaataaatcattcttaaatttgatctgaaatcaggcgggattacccgctgaacttaagcatatctaaaaaggcgggggggaa

SEQ ID NO: 120 - DNA, 645 bp, Mucor hiemalis
  Namadji I isolate 10
tctggtaagtgactgcggaagacattaataatttagatgacctccctagaacttgttctagttaggttcattcttttttactgtgaactcgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaactgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatctaaagtggggggggaaa

SEQ ID NO: 121 - DNA, 659 bp, Mortierella elongata
  Namadji I isolate 11
ttcgactagtgaacttgccggaaggatcattcataataagtgtttttattggcactttttttttaaattccatattccacctttgtgtgcaatgtcagtcgatcttcttcatggagatcgaccaaacatcaacctttattttttaactctttgtctgaaaaatattatgaataaacaattcaaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcttttggattttttaatcgaaaagctttggacttgagcaatcccaacaccaatctttttgagatcggtggcgggttgcttgaaatgcaggtgcagctggacattctcctgagctaaaagcatattcatttagtcccgtcaaacggattattacttttgctgcagctaacataaagggagtttgaccgtattggctgactgatgcaggatttcacaagggtcggcaacgattcttgttaaactcgatctcaaatcaagtaagactacccgctgaacttaagcattaaaaagggggggggagaa

SEQ ID NO: 122 - DNA, 639 bp, Mucor hiemalis Namadji I isolate 12
  Namadji I isolate 12
cctgtagtgactgcggagacattaataatttagatgacctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaattgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatcaaaaaccggggggaaa

SEQ ID NO: 123 - DNA, 640 bp, Mucor hiemalis
  Namadji I isolate 14
cactgtaagtgactgcggagacattaataatttagatggcctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaattgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatcaaaaaaccgggggaa

SEQ ID NO: 124 - DNA, 639 bp, Mucor hiemalis
  Namadji I isolate 15
tctgtagtgactgcggagacattaataatttagatggcctcctagaacttgttctagttaggttcattcttttttactgtgaactgttttaattttcagtgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttattgtgaatggaattgagaatttcgacttgtttgaattctttaaacctattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatcaaaaaaccgggggaaa

SEQ ID NO: 125 - DNA, 639 bp, Mucor hiemalis
  Namadji I isolate 21
cctgaaggtgactgcggagacattaataatttagatggcctcctagaacttgttctagttaggttcattcttttttactgtgaactgttttaattttcagcgtttgaggaatgtcttttagtcatagggatagactattagaatgttaaccgagctgaagtcaggtttagacctggtatcctattaattatttaccaaaagaattcagtattattattgtaacataagcgtaaaaaacttataaaacaacttttaacaacggatctcttggttctcgcatcgatgaagaacgtagcaaagtgcgataactagtgtgaattgcatattcagtgaatcatcgagtctttgaacgcaacttgcgctcaatggtattccattgagcacgcctgtttcagtatcaaaaacaccccacattcataattttgttgtgaatggaactgagaatttcgacttgtttgaattctttaaacttattaggcctgaactattgttctttctgcctgaacatttttttaatataaaggaatgctctagtattaagactatctctggggcctcccaaataaaactttcttaaatttgatctgaaatcaggcgggattacccgctgaacttaagcatatcaaaaaaccgggggaa

SEQ ID NO: 126 - DNA, 637 bp, Mortierella sp.
  Namadji II isolate 1
ttggacagtgaactgcggaagatcattcataataagtgtttatggcacttccttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcataataaaaacgggggggagaaa

SEQ ID NO: 127 - DNA, 640 bp, Mortierella sp.
  Namadji II isolate 2
tcgaatagtgaacttgccggaaggatcattcataataagtgtttattggcactttcttaaattccatattccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatacaaaacggggggaaaac

SEQ ID NO: 128 - DNA, 629 bp, Mortierella sp.
  Namadji II isolate 3
cctgtagtgactgcggagacattcataataagtgtttatggcacttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatcaaaaaggggggggaa

SEQ ID NO: 129 - DNA, 640 bp, Mortierella sp.
  Namadji II isolate 4
tcgaatagtgaacttgcggaagatcattcataataagtgttttatggcacttttccttaaatcccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatacaaaaaaggggggggaaa

SEQ ID NO: 130 - DNA, 640 bp, Mortierella sp.
  Namadji II isolate 5
ttcgaatagtgaacttgcggaaggacattcataataagtgttttatggcactttcttaaatcccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaagggggggagaaaa

SEQ ID NO: 131 - DNA, 630 bp, Mortierella sp.
  Namadji II isolate 6
ctgtcagtgactgcggagacattcataataagtgtttatggcacttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatctaaaaggggggggaaa

SEQ ID NO: 132 - DNA, 646 bp, Mortierella sp.
  Namadji II isolate 7
ttcgaataggtgaacttgccggaaggatcattcataataagtgttttattggcactttccttaaattccatattccacctttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaaggggggggaaaa

SEQ ID NO: 133 - DNA, 630 bp, Mortierella sp.
  Namadji II isolate 8
tcctgtagtgactgcggagacattcataataagtgtttatggcacttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatcaaaaaacggggggaa

SEQ ID NO: 134 - DNA, 643 bp, Mortierella elongata
  Namadji II isolate 9
tcgttaggtgactgcggagacattcataataagtgttttatggcactttttaaatccatatccaccttgtgtgcaatgtcagtcgatcttcttcatggagatcgaccaaacatcaacctttatcttttaactctttgtctgaaaaatattatgaataaacaattcaaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcttttggattttttaatcgaaaagctttggacttgagcaatcccaacaccaatctttttgagatcggtggcgggttgcttgaaatgcaggtgcagctggacattctcctgagctaaaagcatattcatttagtcccgtcaaacggattattacttttgctgcagctaacataaagggagtttgaccgtattggctgactgatgcaggatttcacaagggtcggcaacgattcttgttaaactcgatctcaaatcaagtaagactacccgctgaacttaagcatatctaaaagcggggggaaa

SEQ ID NO: 135 - DNA, 652 bp, Mortierella sp.
  Namadji II isolate 10
gcggtagtgactgcggagacattcataataagtgttttatggcacttttaaaaatccatatccaccttgtgtgaaatgtctgtcgcttttctcttttttccttggaaaaaaacgaagcggctcaacatcaactcattttaattactttgtctgaaaactattatgaataaactaattcaaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaacactctcgatttgtttcgaatgagtattggatttgagcaaacccaacgcctttctgctagccagacttgcggcgggttgcttgaaatgcaggtgcagctgagttttcttctgagctaatagcatacttaattagtcccgtaaaaaggattattaatctgctgaagcttacaccaaaggaagagttctctggctgactgatgcagaattacgcatagccggtaaaacggctttgtaaaactcgatctcaaatcaagtaagactacccgctgaacttaagcatatcaaaaaaccgggggaaa

SEQ ID NO: 136 - DNA, 633 bp, Mortierella sp.
  Namadji II isolate 11
ttgttaggttactgcggaagacattcataataagtgtttatggcactttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatctaaaaggggggggaaa

SEQ ID NO: 137 - DNA, 639 bp, Mortierella sp.
  Namadji II isolate 12
tcgactgtgaactgccggaagatcttcataataagtgtttatggcactttcttaaatcctatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagctttcttcgtaaaactcgatctcaaatcaagtaagactacccgctgaactttaagcatttaaaaatgggggaaaaaatgc

SEQ ID NO: 138 - DNA, 638 bp, Mortierella sp.
  Namadji II isolate 13
tcgaacagtgaactgccggaaggatcattcataataagtgtttatggcactttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaagggggggagaaaa

SEQ ID NO: 139 - DNA, 644 bp, Mortierella sp.
  Namadji II isolate 14
ttcgaatagtgaacttgcggaaggatcattcataataagtgttttattggcactttccttaaattccatattccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaacggggggagaaaa

SEQ ID NO: 140 - DNA, 644 bp, Mortierella sp.
  Namadji II isolate 15
ttcgaatagtgaacttgccggaaggacattcataataagtgttttattggcactttccttaaattccatattccacctttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaaggggggggaaaa

SEQ ID NO: 141 - DNA, 641 bp, Mortierella sp.
  Namadji II isolate 16
tcgaatagtgaacttgccggaaggatcattcataataagtgtttatggcactttcttaaattccatattccacctttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcattaaaaaagggggggaaaaa

SEQ ID NO: 142 - DNA, 640 bp, Mortierella sp.
  Namadji II isolate 17
ttcgacagtgaacttgccggaaggatcattcataataagtgttttatggcactttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttccttgtaaaactcgatctcaaatcaagtaagactacccgctgaacttaagcatttaaaacggggggaggaaa

SEQ ID NO: 143 - DNA, 645 bp, Mortierella sp.
  Namadji II isolate 18
tcgaatagtgaacttgccggaaggatcattcataataagtgttttattggcacttttccttaaattccatattccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatacaaaaaggggggggggaaa

SEQ ID NO: 144 - DNA, 643 bp, Mortierella sp.
  Namadji II isolate 19
ttcggaatagtgaactgcggaagatcattcataataagtgttttatggcacttttccttaaattccatattccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatttaaaaagggggggggaaaa

SEQ ID NO: 145 - DNA, 629 bp, Mortierella sp.
  Namadji II isolate 20
tctgtagtgactgcggagacattcataataagtgtttatggcacttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatcaaaaagcggggggaa

SEQ ID NO: 146 - DNA, 628 bp, Mortierella sp.
  Namadji II isolate 21
gcgtagtgactgcggagacattcataataagtgtttatggcacttcttaaatccatatccaccttgtgtgcaatgtcagccgttttctttatgaaagcggccaaacatcaacttattttttaactgtctgtctgaaaactattatgaataaacaattcaaatacaactttcaacaacggatctcttggctctcgcatcgatgaagaacgcagcgaaatgcgatacgtaatgtgaattgcagaattcagtgaatcatcgaatctttgaacgcatattgcgctctttggtattccgaagagcatgcttgtttgagtatcagtaaacacctcaaagcctttaattcagtttgaaagctttggacttgagcaatcccaacaccagtctcgcgactggcggagggctgcttgaaatgcaggtgcagctggacattctcctgagctataagcatatttatttagtcccgtcaaacggattattacttttgctgcagctaacataaagggagtctgaccgtattggctgactgatgcaggatttcacaagtgctggcaacagcttcttgtaaaactcgatctcaaatcaagtaagactacccgctgaacttaagcatatcaaaaagcggggggaa

SEQ ID NO: 147 - DNA, 22 bp, Artificial sequence
  Oligonucleotide primer
ggaagtaaaagtcgtaacaagg

SEQ ID NO: 148 - DNA, 19 bp, Artificial sequence
  Oligonucleotide primer
tccccgcttattgatatgc

(19) United States
(12) Patent Application Publication
(10) Pub. No.: US 2025/0268292 A1
(43) Pub. Date: Aug. 28, 2025

(54) PRODUCTION OF PHOSPHOLIPIDS IN MICROBES AND USES THEREOF

(71) Applicant: Nourish Ingredients Pty Ltd, Mitchell (AU)

(72) Inventors: Anna EL TAHCHY, Moncrieff (AU); Dawar HUSSAIN, Franklin (AU); Surinder Pal SINGH, Downer (AU); Pushkar SHRESTHA, Lawson (AU); Rosangela Aparecida DEVILLA, Casey (AU); Robert Charles DE FEYTER, Monash (AU); James Robertson PETRIE, Goulburn (AU); Hanh NGUYEN, Harrison (AU); Heinrich KROUKAMP, Asquith (AU)

(73) Assignees: Nourish Ingredients Pty Ltd, Mitchell, AC (AU); Nourish Ingredients Pty Ltd, Mitchell, AC (AU)

(21) Appl. No.: 18/280,188

(22) PCT Filed: Mar. 3, 2022
(86) PCT No.: PCT/AU2022/050177

§ 371 Date: Sep. 1, 2023

(30) Foreign Application Priority Data
Mar. 3, 2021 (AU) 2021900593
Oct. 20, 2021 (AU) 2021903366
Oct. 20, 2021 (AU) 2021903367
Dec. 22, 2021 (AU) 2021904195
Dec. 22, 2021 (AU) 2021904213

(51) Int. Cl. A23L 27/26; A23K 20/142; A23K 20/158; A23K 20/163; A23L 2/56; A23L 27/21; C12N 1/14; C12R 1/645; C12R 1/785

(52) U.S. Cl. CPC A23L 27/26; A23K 20/142; A23K 20/158; A23K 20/163; A23L 2/56; A23L 27/215; C12N 1/145; C12R 2001/645; C12R 2001/785

(57) ABSTRACT

The present invention relates to extracted microbial lipids, microbial cells comprising the lipid, and extracts thereof. The present invention also relates to use of these lipids, cells and extracts in foods, feedstuffs and beverages.

### CROSS REFERENCE TO RELATED APPLICATIONS

This application is a national stage application under 35 U.S.C. § 371 of International Application No. PCT/AU2022/050177, filed internationally on Mar. 3, 2022, which claims priority to and the benefit of Australian Patent Application No. 2021900593, filed on Mar. 3, 2021, Australian Patent Application No. 2021903366, filed on Oct. 20, 2021, Australian Patent Application No. 2021903367, filed on Oct. 20, 2021, Australian Patent Application No. 2021904195, filed on Dec. 22, 2021, Australian Patent Application No. 2021904213, filed on Dec. 22, 2021, the disclosures of which are incorporated herein by reference in their entireties.

### INCORPORATION BY REFERENCE OF SEQUENCE LISTING

The present application is being filed along with a Sequence Listing in electronic format. The Sequence Listing is provided as a file entitled 229752010300SubSeqList.txt, created Sep. 16, 2024, which is 299,818 bytes in size. The information in the electronic format of the Sequence Listing is incorporated by reference in its entirety.

### FIELD OF THE INVENTION

The present invention relates to extracted microbial lipids, microbial cells comprising the lipid, and extracts thereof. The present invention also relates to use of these lipids, cells and extracts in foods, feedstuffs and beverages.

### BACKGROUND OF THE INVENTION

As the global population surges towards a predicted 9 billion people by 2050, the demand for meat and dairy products for human nutrition is expected to continue to increase. However, meat and dairy production worldwide account for 70% of freshwater consumption, 38% of the total arable land use and contribute 19% of the world's greenhouse gas emissions. There is growing interest in finding alternative sources of protein and fat which have less of an environmental footprint. There is also a growing market worldwide for non-animal sources of high-quality protein and fat, for example from plant sources, which are seen as being more sustainable and environmentally friendly. Cultural and religious reasons have also contributed to growing markets for non-animal proteins. However, many current plant-based alternatives for meat and dairy products use fats made from blends of plant oils such as coconut, soy and palm oils which may give inadequate flavour and function. Fats and oils add flavour, lubricity and texture to foods and contribute to the feeling of satiety upon consumption, and therefore food and beverage products incorporating lipids from animal sources are often still preferred by consumers.

The aroma and flavour characteristics of cooked meat are important factors for the eating quality of meat, correlating highly with the acceptance and preference by consumers. The aroma and flavour characteristics come from a large number of volatile and non-volatile compounds which are produced during heating of the meat such as by cooking or roasting (see, for example, the reviews by Dashdorj et al. (2015) and Mottram (1998)). These compounds result from several types of chemical reactions, namely Maillard reactions of amino acids or peptides with reducing sugars, lipid oxidation, the interaction between the Maillard reaction products with the lipid-oxidation products, and degradation of other compounds such as some sulphur-containing compounds during cooking or roasting. The reaction products, particularly the volatile ones, are organic and of low molecular weight, including aldehydes, ketones, alcohols, esters, aliphatic hydrocarbons, thiazoles, oxazoles and pyrazines as well as oxygenated heterocyclic compounds such as lactones and alkylfurans. Many of these compounds do not arise during the cooking of meat-substitutes made with plant proteins and fats such as coconut, soy and palm oils, leading to less consumer acceptance of these non-animal products.

There remains a need for alternative, non-animal sources of lipids that have the ability to provide meat-like flavour and aroma, for human foods and nutrition.

### SUMMARY OF THE INVENTION

The present application is predicated, at least in part, on the surprising determination that certain microbial polar lipids (e.g. phospholipids), can impart a meat-associated flavour and/or aroma to a foodstuff. The present inventors have produced and/or extracted lipids from microbes which comprise ω6 fatty acids in the polar lipid. While these resemble certain animal fat compositions (e.g. beef and pork fats), they differ from animal fats in the types and ratios of ω6 fatty acids and other fatty acids, as well as in the types and ratios of phospholipid classes. Despite these differences, the inventors found that, when heated in the presence of a sugar, an amino acid or other compounds, the extracted lipids mimicked the function of meat lipids and produced meat-like aromas and/or flavours.

As determined herein, extracted microbial lipids that contain predominantly polar lipid that comprises a total fatty acid (TFA) content which comprises the ω6 fatty acid arachidonic acid (ARA), also optionally γ-linolenic acid (GLA) and dihomo-γ-linolenic acid (DGLA), also optionally eicosadienoic acid (EDA), docosatetraenoic acid (DTA) and/or docosapentaenoic acid-ω6 (DPA-ω6), in amounts and ratios that are distinct from those present in meat polar lipids nonetheless produce meat-like aromas and/or flavours when heated in the presence of a sugar and an amino acid. Advantageously, in some embodiments, the extracted microbial lipids also contain relatively low levels of saturated fatty acids, such as palmitic acid, thereby providing a healthy alternative to meat lipids or lipids that more closely mimic meat lipids.

Thus, provided herein are, for example, extracted microbial lipids; compositions that comprise the extracted microbial lipids, an amino acid and a sugar (e.g. flavouring compositions, which can be added to a food or food consumable ingredients so as to form a food); foods and feedstuffs that comprise the extracted microbial lipid, an amino acid and a sugar (e.g. foods that are intended as meat substitutes, such as plant-based burgers, sausages, etc.), and processes and methods for using the extracted microbial lipids to produce compositions, foods and feedstuffs. As a result of the presence of the extracted lipid, sugar and amino acid in the compositions and foods and feedstuffs, the compositions and foods and feedstuffs of the present disclosure will have a meat-like flavour and/or aroma when heated (e.g. produce two or more meat-associated volatile compounds).

In one aspect provided is a composition, comprising an amino acid or derivative, a sugar, and an extracted microbial lipid comprising esterified fatty acids in the form of either (i) polar lipid without any non-polar lipid, or (ii) polar lipid and non-polar lipid, the polar lipid being present in the extracted microbial lipid in a greater amount than the non-polar lipid, wherein
(a) the polar lipid of (i) and (ii) comprises a total fatty acid (TFA) content which comprises ω6 fatty acids, wherein at least some of the ω6 fatty acids are esterified in the form of phospholipids in the polar lipid, the ω6 fatty acids comprising arachidonic acid (ARA), dihomo-γ-linolenic acid (DGLA), and γ-linolenic acid (GLA), wherein ARA is present in an amount of about 10% to about 60% (or at least about 10%, at least about 15%, at least about 20%, at least about 25%, at least about 30%, at least about 35%, at least about 40%, at least about 45%, at least about 50% or at least about 55%) of the total fatty acid content of the polar lipid, DGLA is present in an amount of about 0.1% to about 5% of the total fatty acid content of the polar lipid and GLA is present in an amount of about 1% to about 10% of the total fatty acid content of the polar lipid,
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis),
   wherein when the composition is heated, one or more compounds which have a meat-associated flavour and/or aroma are produced.

In some examples, ARA is present in an amount of about 20% to about 50% (e.g. about 25% to about 50%, or about 30% to about 50%) of the total fatty acid content of the polar lipid, DGLA is present in an amount of about 1% to about 5% of the total fatty acid content of the polar lipid and GLA is present in an amount of about 3% to about 10% of the total fatty acid content of the polar lipid.

In other examples, ARA is present in an amount of about 10% to about 20% of the total fatty acid content of the polar lipid, DGLA is present in an amount of about 0.5% to about 5% of the total fatty acid content of the polar lipid and GLA is present in an amount of about 3% to about 10% of the total fatty acid content of the polar lipid.

Also provided is a composition comprising an amino acid or derivative, a sugar, and an extracted microbial lipid comprising esterified fatty acids in the form of either (i) polar lipid without any non-polar lipid, or (ii) polar lipid and non-polar lipid, the polar lipid being present in the extracted microbial lipid in a greater amount than the non-polar lipid, wherein
(a) the polar lipid comprises a total fatty acid (TFA) content which comprises ω6 fatty acids, wherein the ω6 fatty acids are present in an amount of about 30% to about 70% (or at least about 30%, at least about 35%, at least about 40%, at least about 45%, at least about 50% at least about 55%, at least about 60%, or at least about 65%) of the total fatty acid content of the polar lipid and wherein at least some of the ω6 fatty acids are esterified in the form of phospholipids in the polar lipid, the ω6 fatty acids comprising arachidonic acid (ARA), dihomo-γ-linolenic acid (DGLA), and γ-linolenic acid (GLA),
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis)
   wherein when the composition is heated, one or more compounds which have a meat-associated flavour and/or aroma are produced.

In some examples, the ω6 fatty acids are present in an amount of about 40% to about 70%, about 40% to about 60%, or about 50% to about 60% of the total fatty acid content of the polar lipid. In particular embodiments, ARA is present in an amount of about 20% to about 50% (e.g about 25% to about 50%, or about 30% to about 50%) of the total fatty acid content of the polar lipid, DGLA is present in an amount of about 1% to about 5% of the total fatty acid content of the polar lipid and GLA is present in an amount of about 3% to about 10% of the total fatty acid content of the polar lipid.

In some examples, ω3 fatty acids are either absent from the polar lipid or are present in a total amount of less than about 3% by weight of the TFA content of the polar lipid, and/or wherein the polar lipid lacks C16:2, C16:3ω3, EPA and DHA.

In one embodiment, the polar lipid comprises myristic acid (C14:0) in an amount of less than about 2% by weight of the total fatty acid content of the polar lipid.

In one embodiment the phospholipids comprising the ω6 fatty acids comprise two, three, or all four of phosphatidylcholine (PC), phosphatidylethanolamine (PE), phosphatidylinositol (PI) and phosphatidylserine (PS), optionally one or more of phosphatidic acid (PA), phosphatidylglycerol (PG) and cardiolipin (Car), preferably comprising at least PC and PE or at least PC, PE, PS and PI, each comprising one or at least two or more of ARA, DGLA, and GLA.

In one example, the phospholipids comprising the ω6 fatty acids comprise phosphatidylcholine (PC) and phosphatidylethanolamine (PE), each comprising one or at least two or more of ARA, DGLA and GLA.

In one embodiment, the phospholipids comprising the ω6 fatty acids comprise phosphatidylcholine (PC) and phosphatidylethanolamine (PE), phosphatidylinositol (PI), phosphatidylserine (PS), and phosphatidic acid (PA), each comprising one or at least two or more of ARA, DGLA and GLA, wherein ARA is present in PC an amount of about 14% to about 20% of the total fatty acid content of the PC, ARA is present in PE an amount of about 15% to about 20% of the total fatty acid content of the PE, and ARA is present in PA an amount of about 15% to about 20% of the total fatty acid content of the PA.

In one example, stearic acid is present at a level of less than about 7% or less than about 6% or less than about 5%, preferably less than 4% or less than 3%, of the total fatty acid content of the polar lipid.

In one embodiment, the extracted microbial lipid is extracted fungal lipid or a eukaryotic microbial lipid.

In one embodiment the extracted microbial lipid is extracted yeast lipid, preferably a Saccharomyces cerevisiae, Yarrowia lipolytica, or Pichia pastoris lipid.

In another embodiment the extracted microbial lipid is extracted Mortierella spp (e.g. M. alpina) lipid.

In one embodiment, at least one of the following apply:
(a) at least one of EDA, DTA and DPA-ω3 is also present in the polar lipid;
(b) the ratio of PC to PE or to phospholipids other than PC is less than 3:1, less than 2:1, less than 1.5:1, less than 1.25:1, less than 1:1, between 3:1 and 1:1, between 2:1 and 1:1, or between 3:1 and 0.5:1.

In one embodiment, the saturated fatty acid content of the polar lipid comprises one or more or all of lauric acid (C12:0), myristic acid (C14:0), a C15:0 fatty acid, C20:0, C22:0 and C24:0, preferably comprising C14:0 and C24:0 or C14:0, C15:0 and C24:0, more preferably comprising C14:0, C15:0 and C24:0 but not C20:0 and C22:0.

In one example, lauric acid and myristic acid are absent from the polar lipid, or lauric acid and/or myristic acid is present in the polar lipid, whereby the sum of the amounts of lauric acid and myristic acid in the polar lipid is less than about 2%, or less than about 1%, preferably less than about 0.5%, more preferably less than about 0.2%, of the total fatty acid content of the polar lipid.

In one embodiment, C15:0 is absent from the polar lipid, or C15:0 is present in the polar lipid in an amount of less than about 3%, preferably less than about 2% or less than about 1%, of the total fatty acid content of the polar lipid.

In one embodiment, wherein palmitic acid is present in the polar lipid in an amount of about 10% to about 20% of the fatty acid content of the polar lipid.

In one embodiment, wherein palmitoleic acid is present in the polar lipid in an amount of about 3% to about 45%, or about 3% to about 25%, or about 3% to about 20%, or about 3% to about 15%, of the total fatty acid content of the polar lipid.

In another embodiment, oleic acid is present in the polar lipid in an amount of about 3% to about 60%, or about 3% to about 40%, or about 3% to about 25%, or about 20% to about 60%, of the total fatty acid content of the polar lipid.

In another embodiment, vaccenic acid is absent from the polar lipid, or vaccenic acid is present in the polar lipid in an amount of less than about 2%, preferably less than about 1% or about 0.5%, of the total fatty acid content of the polar lipid.

In one embodiment linoleic acid is present in the polar lipid in an amount of about 3% to about 20%, of the total fatty acid content of the polar lipid.

In another embodiment, eicosadienoic acid is absent from the polar lipid, or eicosadienoic acid is present in the polar lipid in an amount of about 3% to about 12%, or about 3% to about 8%, or about 3% to about 6%, or less than about 3%, of the total fatty acid content of the polar lipid.

In a further embodiment, C20:0 and C22:0 are absent from the polar lipid, or C20:0 and/or C22:0 is present in the polar lipid, whereby the sum of the amounts of C20:0 and C22:0 in the polar lipid is less than about 1.0%, less than about 0.5%, preferably less than 0.2%, of the total fatty acid content of the polar lipid.

In another embodiment, C24:0 is absent from the polar lipid, or C24:0 is present in the polar lipid in an amount of less than about 1.0%, less than 0.5%, preferably less than 0.3% or less than 0.2%, of the total fatty acid content of the polar lipid.

In another embodiment, C17:1 is absent from the polar lipid, or C17:1 is present in the polar lipid in an amount of less than about 5%, preferably less than about 4% or less than about 3%, more preferably less than about 2% of the total fatty acid content of the polar lipid.

In another embodiment, wherein monounsaturated fatty acids which are C20 or C22 fatty acids are absent from the polar lipid, or C20:1 and/or C22:1 is present in the polar lipid, whereby the sum of the amounts of C20:1 and C22:1 in the polar lipid is less than about 1.0%, less than about 0.5%, preferably less than 0.2%, of the total fatty acid content of the polar lipid.

In another embodiment, wherein the content of ω6 fatty acids in the polar lipid which are (i) C20 or C22 fatty acids is about 5% to about 60%, preferably about 10% to about 60% of the total fatty acid content of the polar lipid, and/or (ii) ω6 fatty acids which have 3, 4 or 5 carbon-carbon double bonds, is about 5% to about 70%, preferably about 10% to about 70%, more preferably about 40% to about 70% or about 45% to about 70% or about 50% to about 70% of the total fatty acid content of the polar lipid.

In another embodiment, wherein C16:3ω3 is absent from the polar lipid, or both C16:2 and C16:3ω3 are absent from the polar lipid.

In another embodiment, the extracted microbial lipid comprises PC and/or lacks cyclopropane fatty acids, preferably which lacks C15:0c, C17:0c and C19:0c.

In another embodiment, the extracted lipid is obtained from a genetically modified microbe. For example, the genetically modified microbe may have one or more genetic modification(s) which provide for
(i) synthesis of, or increased synthesis of, one or more ω6 fatty acids in the microbe,
(ii) an increase in total fatty acid synthesis and/or accumulation in the microbe,
(iii) an increase in total polar lipid synthesis and/or accumulation in the microbe,
(iv) a decrease in triacylglycerol (TAG) synthesis and/or accumulation in the microbe, or an increase in TAG catabolism in the microbe, preferably an increase in TAG lipase activity,
(v) a reduction in catabolism of total fatty acids in the microbe, or any combination thereof.

In another embodiment, the genetic modification(s) provide for at least two of (i) to (v), preferably (iv) and (v), or (i), (iv) and (v).

In an embodiment, wherein when the composition is heated, the heat is at least about 100° C., preferably at least about 120° C., more preferably at least about 140° C.

Also provided is a composition, comprising an amino acid or derivative, a sugar, and an extracted Mortierella spp. lipid comprising esterified fatty acids in the form of either (i) polar lipid without any non-polar lipid, or (ii) polar lipid and non-polar lipid, the polar lipid being present in the extracted microbial lipid in a greater amount than the non-polar lipid. In some examples, the extracted Mortierella spp. lipid is an extracted Mortierella alpina lipid. The composition may also further comprise another food, feedstuff or beverage ingredient.

In some embodiments of the compositions of the present invention, the sugar, sugar alcohol, sugar acid, or sugar derivative is selected from ribose, xylose, glucose, fructose, sucrose, arabinose, glucose-6-phosphate, fructose-6-phosphate, fructose 1,6-diphosphate, inositol, maltose, molasses, altodextrin, glycogen, galactose, lactose, ribitol, gluconic acid and glucuronic acid, amylose, amylopectin, or any combination thereof, preferably wherein the sugar is ribose or xylose.

In further embodiments, the amino acid or derivative thereof is selected from cysteine, cystine, a cysteine sulfoxide, allicin, selenocysteine, methionine, isoleucine, leucine, lysine, phenylalanine, threonine, tryptophan, 5-hydroxytryptophan, valine, arginine, histidine, alanine, asparagine, aspartate, glutamate, glutamine, glycine, proline, serine, tyrosine, or any combination thereof, preferably wherein the amino acid or derivative thereof is a sulfur-containing amino acid or derivative.

In other embodiments, the composition further comprises one or more fatty acids, esterified or non-esterified, from a source other than the extracted microbial lipid, cell or extract.

In some examples, the composition is in the form of a powder, solution, suspension, or emulsion.

In one example, the composition comprises less than 5%, less than 10%, less than 15% or less than 20% (w/w or w/v) protein.

In one embodiment, the composition comprises, per gram of dry composition or slurry, or per ml of liquid composition, at least about 5 mg, at least about 10 mg, at least about 15 mg, at least about 20 mg, at least about 25 mg, or at least about 50 mg extracted microbial lipid.

In one embodiment, the composition comprises, per gram of dry composition or slurry, or per ml of liquid composition, from about 10 mg to about 100 mg extracted microbial lipid or from about 15 mg to about 50 mg extracted microbial lipid.

Also provided is a food, feedstuff or beverage comprising an ingredient which comprises a composition as described herein, and at least one other food, feedstuff or beverage ingredient.

In another aspect, provided is a food, feedstuff or beverage comprising extracted Mortierella spp. lipid (e.g. extracted M. alpina lipid), wherein the lipid comprises esterified fatty acids in the form of either (i) polar lipid without any non-polar lipid, or (ii) polar lipid and non-polar lipid, the polar lipid being present in the extracted microbial lipid in a greater amount than the non-polar lipid, and wherein the food, feedstuff or beverage further comprises an amino acid or derivative, and a sugar, and at least one other food, feedstuff or beverage ingredient.

In a further aspect, provided is a food, feedstuff or beverage comprising an ingredient which is the extracted microbial lipid as defined above and herein, wherein the food, feedstuff or beverage further comprises an amino acid or derivative, and a sugar, and at least one other food, feedstuff or beverage ingredient.

In another aspect, provided is food, feedstuff or beverage comprising lipids and at least one other food, feedstuff or beverage ingredient, wherein the lipids are a product of a reaction between an extracted microbial lipid of the invention, an amino acid or derivative, and a sugar under conditions sufficient to produce at least two compounds which have a meat-associated flavour and/or aroma.

In some examples, the sugar, sugar alcohol, sugar acid, or sugar derivative in the food, feedstuff or beverage is selected from ribose, xylose, glucose, fructose, sucrose, arabinose, glucose-6-phosphate, fructose-6-phosphate, fructose 1,6-diphosphate, inositol, maltose, molasses, altodextrin, glycogen, galactose, lactose, ribitol, gluconic acid and glucuronic acid, amylose, amylopectin, or any combination thereof, preferably wherein the sugar is ribose or xylose.

In one embodiment, the amino acid or derivative thereof in the food, feedstuff or beverage is selected from cysteine, cystine, a cysteine sulfoxide, allicin, selenocysteine, methionine, isoleucine, leucine, lysine, phenylalanine, threonine, tryptophan, 5-hydroxytryptophan, valine, arginine, histidine, alanine, asparagine, aspartate, glutamate, glutamine, glycine, proline, serine, tyrosine, or any combination thereof, preferably wherein the amino acid or derivative thereof is a sulfur-containing amino acid or derivative.

In one embodiment, the at least one other food, feedstuff or beverage ingredient comprises a protein (e.g. a microbial protein or plant protein), optionally wherein the composition comprises at least 10% by weight protein.

In some embodiments, the food, feedstuff or beverage has no components obtained from an animal. In other embodiments, the food, feedstuff or comprises components obtained from an animal, e.g. components that comprise meat.

Also provided is a food or feedstuff comprising at least two meat-associated flavour and/or aroma compounds derived from an extracted microbial lipid as defined herein, or a composition of the invention, wherein the food, feedstuff or beverage comprises a greater amount of the at least two compounds which have a meat-associated flavour and/or aroma than a corresponding food, feedstuff or beverage which was produced with a corresponding lipid or composition lacking the polar lipid comprising the ω6 fatty acid(s).

In an embodiment, the corresponding lipid of the corresponding food, feedstuff or beverage may comprise lipids (e.g., non-polar lipids) other than the polar lipid comprising the ω6 fatty acid(s).

In an embodiment, the corresponding lipid of the corresponding food, feedstuff or beverage does not comprise esterified fatty acids in the form of either (i) polar lipid without any non-polar lipid, or (ii) polar lipid and non-polar lipid, the polar lipid being present in the extracted microbial lipid in a greater amount than the non-polar lipid, wherein
(a) the polar lipid of (i) and (ii) comprises a total fatty acid (TFA) content which comprises ω6 fatty acids, wherein at least some of the ω6 fatty acids are esterified in the form of phospholipids in the polar lipid, the ω6 fatty acids comprising arachidonic acid (ARA), dihomo-γ-linolenic acid (DGLA), and γ-linolenic acid (GLA), wherein ARA is present in an amount of about 10% to about 60% of the total fatty acid content of the polar lipid, DGLA is present in an amount of about 0.1% to about 5% of the total fatty acid content of the polar lipid and GLA is present in an amount of about 1% to about 10% of the total fatty acid content of the polar lipid,
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis).

In an embodiment, the corresponding lipid of the corresponding food, feedstuff or beverage does not comprise esterified fatty acids in the form of either (i) polar lipid without any non-polar lipid, or (ii) polar lipid and non-polar lipid, the polar lipid being present in the extracted microbial lipid in a greater amount than the non-polar lipid, wherein
(a) the polar lipid of (i) and (ii) comprises a total fatty acid (TFA) content which comprises ω6 fatty acids, wherein the ω6 fatty acids are present in an amount of about 30% to about 70% of the total fatty acid content of the polar lipid and wherein at least some of the ω6 fatty acids are esterified in the form of phospholipids in the polar lipid, the ω6 fatty acids comprising arachidonic acid (ARA), dihomo-γ-linolenic acid (DGLA), and γ-linolenic acid (GLA),
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis).

In one example, the food or feedstuff is a meat substitute.

In a particular example, applying heat to the food, feedstuff or beverage results in the production of one or more compound(s) which have a meat-associated flavour and/or aroma, preferably volatile compounds.

In reference to the composition, food, feedstuff or beverage as described herein, applying heat to the composition, food, feedstuff or beverage can result in the production of two or more volatile compound(s) selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, and 1,3,5-thitriane.

In some examples, applying heat to the composition, food, feedstuff or beverage results in the production of two or more volatile compound(s) selected from 2-heptanone, 3-octanone, 2,3-octanedione, 1-pentanol, 1-hexanol, 2-ethyl-1-hexanol, 1-octanol, trans-2-octen-1-ol and 1-nonanol.

In further examples, applying heat to the composition, food, feedstuff or beverage results in the production of two or more volatile compound(s) selected from 1-pentanal, 3-octanone, 2-octen-1-ol, 1-nonanol and 1-octanol, and optionally 1,3-bis(1,1-dimethylethyl)-benzene.

In one embodiment, applying heat to the composition, food, feedstuff or beverage results in the production of two or more volatile compound(s) selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; and 2,3,4,5-Tetramethylcyclopent-2-en-1-ol.

In one embodiment, the food, feedstuff or beverage is or has been heated, optionally at a temperature of at least about 100° C., preferably at least about 120° C., more preferably at least about 140° C.

Also provided is a method of producing a food, feedstuff or beverage, the method comprising combining a composition of the invention, with at least one other food, feedstuff or beverage ingredient.

In another aspect, provided is a method of producing a food, feedstuff or beverage, the method comprising combining an extracted microbial lipid as defined herein, optionally wherein the extracted microbial lipid has been heated at a temperature of at least about 100° C., at least about 120° C. or at least about 140° C., with a sugar, an amino acid or derivative, and at least one other food, feedstuff or beverage ingredient.

In a further aspect, provided is a method of preparing a food, feedstuff or beverage for consumption, the method comprising heating a food, feedstuff or beverage of the invention to produce a chemical reaction between fatty acids, sugars and amino acids in the food, feedstuff or beverage.

Also provided is a method of increasing a meat-associated flavour and/or aroma of a food, feedstuff or beverage, comprising heating a food, feedstuff or beverage comprising an extracted microbial lipid as defined herein, or a composition of the invention, and at least one other food, feedstuff or beverage ingredient, under conditions sufficient to produce meat-associated flavour and/or aroma compounds.

In some examples of the above methods, the food, feedstuff or beverage is heated at a temperature of at least about 100° C., preferably at least about 120° C., more preferably at least about 140° C.

Also provided is the use of an extracted microbial lipid as defined herein, or a composition the invention, to produce a food, feedstuff or beverage ingredient, or a food, feedstuff or beverage.

Also provided is isolated strain of Mortierella sp. selected from:
i) yNI0125 deposited under V21/019953 on 12 Oct. 2021 at the National Measurement Institute Australia;
ii) yNI0126 deposited under V21/019951 on 12 Oct. 2021 at the National Measurement Institute Australia;
iii) yNI0127 deposited under V21/019952 on 12 Oct. 2021 at the National Measurement Institute Australia; and
iv) yNI0132 deposited under V21/019954 on 12 Oct. 2021 at the National Measurement Institute Australia.

In another aspect, the present invention provides a microbial cell extract comprising lipid of the invention or produced from the microbial cell of the invention, comprising polar lipid which comprises ω6 fatty acids esterified in the form of phospholipids. The extract may be produced by any means known in the art, including, for example, by culturing the microbial cells, breaking the cell wall (e.g., by heating the cells or lysing the cell walls), and optionally centrifuging and/or concentrating (e.g., by evaporation) the resulting lysate.

In another aspect, the present invention provides a process for producing extracted lipid, comprising extracting lipid from the microbial cells of the invention, for example
(a) obtaining microbial cells of the invention, and
(b) extracting lipid from the microbial cells, so as to thereby produce the extracted lipid.

Suitable methods for extracting lipids from microbial cells are described herein. For example, the lipid can be extracted by any means known in the art such as, but not limited to, exposing the cells to an organic solvent, pressing the cells or treating the cells with microwave irradiation, ultrasonication, high-speed homogenization, high-pressure homogenization, bead beating, autoclaving, thermolysis or any combination thereof.

In one embodiment, the method further comprises culturing the cells.

In one embodiment, the cells are cultured in a medium comprising an ω6 fatty acid, preferably one or more of LA, GLA, DGLA, EDA, ARA, DTA or DPAω6.

In one embodiment, the ω6 fatty acids are free fatty acids or fatty acid salts.

In one embodiment, the cells are cultured in a medium lacking ω6 fatty acids, preferably a medium lacking ω6 other than LA, or a medium comprising oleic acid and/or glycerol, preferably oleic acid and glycerol.

In one embodiment, the method further comprises modifying or purifying the lipid, preferably modifying the lipid by one or more of reducing the amount of one or more non-polar lipids and/or free fatty acids, increasing the amount of one or more ω6 fatty acids in the total fatty acid content of the lipid, increasing the amount of total ω6 fatty acids in the total fatty acid content of the lipid, reducing the amount of total saturated fatty acids in the total fatty acid content of the lipid, or altering the ratio of one or more of PC:PE, PC:PI or PC:PS. The ratio of one or more of PC:PE, PC:PI or PC:PS can also be altered by adjusting the culture conditions prior to lipid extraction.

In one embodiment, the method further comprises purifying the polar lipid from the extracted microbial lipid, preferably reducing the amount of one or more of TAG, DAG, free fatty acids, protein, carbohydrate, waxes, pigments or volatile compounds. For example, purifying the polar lipid can be performed using known solvent extraction and fractionation methods.

In another aspect, the present invention provides a process for culturing microbial cells, the process comprising
(a) obtaining microbial cells of the invention, and
(b) increasing the number of the cells by culturing the cells in a suitable medium.

In another aspect, the present invention provides a process for producing a microbial cell which produces lipid of the invention, preferably which produces an increased amount of said lipid relative to a progenitor microbial cell, the process comprising a step of introducing one or more genetic modifications and/or exogenous polynucleotides as defined above into a progenitor microbial cell.

In one embodiment, the process comprises one or more steps of
(i) producing progeny cells from the cell comprising the introduced genetic modifications and/or exogenous polynucleotides,
(ii) mutagenesis of a population of progenitor cells,
(iii) introduction of one or more exogenous polynucleotides whereby the exogenous polynucleotides become integrated into the genome of the microbial cell, preferably into one or more predetermined locations,
(iv) determining the fatty acid composition of the cell or progeny cells thereof, and
(v) selecting a progeny cell which comprises lipid of the invention.

In another aspect, the present invention provides a composition comprising one or more or all of the lipid of the invention, the microbial cell of the invention or the microbial cell extract of the invention, and one, two or all three of (i) a sugar, sugar alcohol, sugar acid, or sugar derivative, (ii) an amino acid or derivative thereof containing a free amino group, and (iii) a sulphur-containing compound other than a sulphur-containing amino acid.

In an embodiment, the present invention provides a composition for producing a food-like aroma and/or flavour when heated, the composition comprising:
a) microorganism biomass containing phospholipids and/or extracted lipids, preferably comprising phospholipids extracted from a microorganism;
b) one or more sugars, sugar alcohols, sugar acids, or sugar derivatives; and
c) one or more amino acids or derivatives or salts thereof.

In embodiments, the composition comprises both microbial biomass containing phospholipids and phospholipids extracted from the microbes. In an embodiment, the dry weight ratio of the microbial biomass to the extracted lipid/phospholipid is between 10:1 and 2:1, between 2:1 and 1:1, between 1:1 and 1:2 or between 1:2 and 1:10. In an embodiment, the extracted lipid/phospholipid is from a microbe different to the microbial biomass.

Such compositions can, in some embodiments, be used to increase a meat-associated flavour and/or aroma of a food, feedstuff or beverage. The composition may be in the form of a powder, solution, suspension, emulsion or other suitable form. Furthermore, the composition may be packaged within a packet, shaker or other receptacle that enables a user to easily add the composition to a food, feedstuff or beverage, or an ingredient thereof.

In one embodiment, the composition further comprises another food, feedstuff or beverage ingredient.

In one embodiment, the sugar, sugar alcohol, sugar acid, or sugar derivative is selected from ribose, xylose, glucose, fructose, sucrose, arabinose, glucose-6-phosphate, fructose-6-phosphate, fructose 1,6-diphosphate, inositol, maltose, molasses, altodextrin, glycogen, galactose, lactose, ribitol, gluconic acid and glucuronic acid, amylose, amylopectin, or any combination thereof, preferably wherein the sugar is ribose or xylose.

In one embodiment, the amino acid or derivative thereof is selected from cysteine, cystine, a cysteine sulfoxide, allicin, selenocysteine, methionine, isoleucine, leucine, lysine, phenylalanine, threonine, tryptophan, 5-hydroxytryptophan, valine, arginine, histidine, alanine, asparagine, aspartate, glutamate, glutamine, glycine, proline, serine, tyrosine, or any combination thereof, preferably wherein the amino acid or derivative thereof is a sulfur-containing amino acid or derivative.

In one embodiment, the composition further comprises one or more fatty acids, esterified or non-esterified, from a source other than the extracted microbial lipid, cell or extract.

In one embodiment, the composition is a dry composition. In another embodiment, the composition is a liquid composition. In one embodiment, the composition is in the form of a powder, solution, suspension, or emulsion.

In another aspect, the present invention provides a food, feedstuff or beverage comprising an ingredient which is one or more or all of the lipid of the invention, the microbial cell of the invention, the microbial cell extract of the invention, or the composition of the invention, and at least one other food, feedstuff or beverage ingredient.

In another aspect, the present invention provides a food, feedstuff or beverage comprising an ingredient which is Mortierella sp. or a homogenate thereof, and at least one other food, feedstuff or beverage ingredient. In an embodiment, the Mortierella sp. is alive. In an embodiment, the Mortierella sp. is dead, for instance the cells may have been heat-treated in order to render them incapable of replicating. In an embodiment, the food, feedstuff or beverage comprises at least 1%, at least 5%, at least 10%, 1% and 20% or between 1% and 50% of the Mortierella sp. or a homogenate thereof. In an embodiment, the Mortierella sp. is genetically modified as defined herein. In an embodiment, the Mortierella sp. is not genetically modified.

In another aspect, the present invention provides a food, feedstuff or beverage comprising an ingredient which is Yarrowia sp. or a homogenate thereof (such as Yarrowia cells described herein, such as, for example, Yarrowia cells comprising polar lipid as defined above or herein), and at least one other food, feedstuff or beverage ingredient. In an embodiment, the Yarrowia sp. is alive. In an embodiment, the Yarrowia sp. is dead, for instance the cells may have been heat-treated in order to render them incapable of replicating. In an embodiment, the food, feedstuff or beverage comprises at least 1%, at least 5%, at least 10%, between 1% and 20% or between 1% and 50% of the Yarrowia sp. or a homogenate thereof. In an embodiment, the Yarrowia sp. is genetically modified as defined herein. In an embodiment, the Yarrowia sp. is not genetically modified.

In one embodiment, any of the the foods, feedstuffs or beverages of the present invention are packaged ready for sale.

In another aspect, the present invention provides a method of producing a food, feedstuff or beverage, the method comprising combining one or more or all of the lipid of the invention, the microbial cell of the invention, the microbial cell extract of the invention, or the composition of the invention, with at least one other food, feedstuff or beverage ingredient, or heating said lipid, cells, extract or composition. For example, the lipid, microbial cell, microbial cell extract or the composition can be combined with the other food or feedstuff or beverage ingredient by mixing, applying it to the surface of the other ingredient, or by soaking/marinating the other ingredient. In an embodiment, the food, feedstuff or beverage is prepared by (a) heating a composition comprising the lipid of the invention and/or the microbial cells of the invention and (b) mixing the products from (a) with other food, feedstuff or beverage ingredients.

In another aspect, the present invention provides a method of preparing a food, feedstuff or beverage for consumption, the method comprising heating a food, feedstuff or beverage of the invention to produce a chemical reaction between fatty acids, sugars and amino acids in the food or feedstuff. In an embodiment, the chemical reaction comprises Maillard reactions.

In another aspect, the present invention provides a method of increasing a meat-associated flavour and/or aroma of a food, feedstuff or beverage when the food, feedstuff or beverage is heated, the method comprising (a) contacting or combining the lipid of the invention, the microbial cell of the invention, the microbial cell extract of the invention, or the composition of the invention with the food, feedstuff or beverage, and optionally (b) heating the food, feedstuff or beverage. Alternatively, the food, feedstuff or beverage is prepared by (a) heating a composition comprising the lipid of the invention and/or the microbial cells of the invention and (b) contacting or mixing the products from (a) with other food, feedstuff or beverage ingredients. In embodiments, the step of contacting or combining the food product, beverage product or feedstuff with the composition comprises one or more or all of mixing, coating, basting, soaking or marinating the food product, beverage product or feedstuff with the composition. In embodiments, the method further comprises a step of grinding, mincing, rolling, chopping, extruding or drying the food product, beverage product or feedstuff after, or simultaneously with, the step of contacting food product, beverage product or feedstuff with the composition, or any combination of these further steps.

In another aspect, the present invention provides a method of increasing a meat-associated flavour and/or aroma of a food, feedstuff or beverage, comprising heating a food, feedstuff or beverage comprising one or more or all of the lipid of the invention, the microbial cell of the invention, the microbial cell extract of the invention, or the composition of the invention, and at least one other food, feedstuff or beverage ingredient, under conditions sufficient to produce meat-associated flavour and/or aroma compounds.

In one embodiment, the food, feedstuff or beverage ingredient is heated at a temperature of at least about 100° C., preferably at least about 120° C., more preferably at least about 140° C. In an embodiment, the heating step is for at least 5 min. In an embodiments, the heating step is for between 5 min and 75 min, preferably between 5 min and 45 min.

In one embodiment, the meat-associated flavour and/or aroma is beef-like, chicken-like, pork-like or fish-like. In a preferred embodiment, the composition provides an umami flavour or aroma, or increases an umami flavour or aroma in a food or beverage product. In preferred embodiments, the composition does not provide a bitterness or sourness to the food product, beverage product or feedstuff.

In another aspect, the present invention provides use of one or more or all of the lipid of the invention, the microbial cell of the invention, the microbial cell extract of the invention, or the composition of the invention to produce a food, feedstuff or beverage ingredient, or a food, feedstuff or beverage, or to increase a meat-associated flavour and/or aroma of a food, feedstuff or beverage.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Polyunsaturated fatty acid biosynthesis pathways.

FIG. 2. Growth curves for S. cerevisiae cultured for up to 7 days in YPD medium

FIG. 3. Graphical representation of volatile compounds identified by GC-MS in reaction mixtures containing the YL ARA and YL polar lipid preparations shown in Table 32. The graph shows the area percentage (%) of total identified compounds for each reaction mixture. Bars not shown for some compound IDs means that compound was not detected in that mixture under the specified analytical conditions.

FIG. 4. Graphical representation of volatile compounds identified by HS-SPME-GCMS in reaction mixtures containing ARA-PC or 18:0/18:1-PC (Con) polar lipids applied at 2.5 or 5.0 mg. The graph shows the percentage (%) for each compound of the total area of identified compounds for each reaction mixture. Bars not shown for a compound means that compound was not detected in that mixture under the specified analytical conditions.

FIG. 5. Schematic representation for making genetic constructs to introduce inactivating deletions into genes of interest such as microbial FAD2 and URA3. Panel A. DNA synthesis of a 2 kb fragment having 1,000 bp 5′ upstream and 1,000 bp 3′ downstream regions of the gene of interest joined with a SacII site between the two regions. The position of restriction sites and lox sites are indicated by vertical lines. CDS: protein coding region of the gene of interest. B. Amplification of hygromycin (Hph) or nourseothricin (Nat1) antibiotic resistance genes using primers adapted with SacII sites. C. Assembly of genetic construct by insertion of the SacII-ended antibiotic resistance gene cassettes into the DNA fragment of A. Not drawn to scale.

FIG. 6. Schematic representation of construction of genetic constructs for introducing gene deletions into microbes. Panel A. PCR amplification of 5′ upstream and 3′ downstream regions of the gene of interest and ligation together to make a 2 kb fragment. Oligonucleotide primers are shown as small horizontal arrows, restriction enzyme sites and lox sites as vertical lines. CDS: protein coding region of the gene of interest. B. Amplification of hygromycin (Hph) or nourseothricin (Nat1) resistance genes using primers adapted with flanking AsiSI sites. C. Assembly of genetic construct for introduction into microbes such as Y. lipolytica.

FIG. 7. Schematic structure of a phospholipid. One of the hydroxyls can be replaced with different headgroups such as choline, serine or inositol.

FIG. 8. Schematic of the pathways for phospholipid synthesis.

FIG. 9 shows the meatiness results of a sensory evaluation of samples comprising a maillard reaction matrix at varying concentrations and Mortierella alpina biomass.

FIG. 10 shows the pleasantness results of a sensory evaluation of samples comprising a maillard reaction matrix at varying concentrations and Mortierella alpina biomass.

FIG. 11 shows the combined meatiness and pleasantness results of a sensory evaluation of samples comprising a maillard reaction matrix at varying concentrations and Mortierella alpina biomass.

### KEY TO THE SEQUENCE LISTING

SEQ ID NO:1 Lachancea kluyveri Δ12 desaturase. Watanabe et al. (2004). Accession No. BAD08375.1; 416aa
SEQ ID NO:2 Y. lipolytica strain W29 endogenous Δ12 desaturase (FAD2), WO2004/101757, Accession No. XP_500707.1; 419aa
SEQ ID NO:3 Acheta domesticus Δ12 desaturase; 357aa. Accession No. ABY26957.1. (Zhou et al., 2008)
SEQ ID NO:4 Fusarium moniliforme Δ12 desaturase; 477aa, Accession No. XP 018751050.1
SEQ ID NO:5 Ostreococcus tauri Δ6-desaturase, 456 aa, Accession No. XP 003082578.1
SEQ ID NO:6 Mortierella alpina Δ6 desaturase; 457aa, Accession No. AAL73949.1.
SEQ ID NO:7 Pavlova pinguis Δ9-elongase, Accession No. ADN94475 (GQ906528); 272aa
SEQ ID NO:8 Pavlova salina Δ9-elongase; 279aa, Petrie et al. (2010). Accession No. GQ906529
SEQ ID NO:9 Isochrysis galbana Δ9-elongase (CAH05232); Napier et al. (2004); 258aa
SEQ ID NO:10 Isochrysis galbana Δ9-elongase, 263aa, Accession No. AAL37626; Qi et al. (2002)
SEQ ID NO:11 Isochrysis galbana Δ9-elongase IgASE2, 261aa, Accession No. ADD51571-Li et al. (2011)
SEQ ID NO:12 Emiliania huxleyi CCMP1516 Δ9-elongase, Accession No. XP 005759783.1, WO2011/006948
SEQ ID NO:13 Pyramimonas cordata CS0140 Δ6 elongase, Accession No. ACR53359.1
SEQ ID NO:14 Pavlova salina Δ8 desaturase, 427aa, Accession No. A4KDP1.1, Zhou et al. (2007)
SEQ ID NO:15 Pavlova salina Δ5 desaturase; 425aa, Accession No. A4KDP0.1
SEQ ID NO:16 Mortierella alpina Δ5 desaturase; 446aa
SEQ ID NO:17 Pyramimonas cordata CS0140 Δ5 elongase, 267aa, Accession No. ACR53360.1, Petrie et al. (2010).
SEQ ID NO:18 Pavlova salina Δ4 desaturase; 447aa (Accession No. A0PJ29.1); Zhou et al. (2007).
SEQ ID NO:19 Thraustochytrium Δ4 desaturase, 519aa; Accession No. CAX48933
SEQ ID NO:20 at003 primer sequence
SEQ ID NO:21 at004 primer sequence
SEQ ID NO:22 at213 primer sequence
SEQ ID NO:23 at214 primer sequence
SEQ ID NO:24 at215 primer sequence
SEQ ID NO:25 at216 primer sequence
SEQ ID NO:26 at217 primer sequence
SEQ ID NO:27 at218 primer sequence
SEQ ID NO:28 at219 primer sequence
SEQ ID NO:29 at220 primer sequence
SEQ ID NO:30 at221 primer sequence
SEQ ID NO:31 at222 primer sequence
SEQ ID NO:32 at223 primer sequence
SEQ ID NO:33 at224 primer sequence
SEQ ID NO:34 at225 primer sequence
SEQ ID NO:35 at226 primer sequence
SEQ ID NO:36 at227 primer sequence
SEQ ID NO:37 at228 primer sequence
SEQ ID NO:38 at229 primer sequence
SEQ ID NO:39 at230 primer sequence
SEQ ID NO:40 at239 primer sequence
SEQ ID NO:41 at240 primer sequence
SEQ ID NO:42 at241 primer sequence
SEQ ID NO:43 at242 primer sequence
SEQ ID NO:44 at243 primer sequence
SEQ ID NO:45 at244 primer sequence
SEQ ID NO:46 at245 primer sequence
SEQ ID NO:47 at246 primer sequence
SEQ ID NO:48 at247 primer sequence
SEQ ID NO:49 at248 primer sequence
SEQ ID NO:50 at249 primer sequence
SEQ ID NO:51 at250 primer sequence
SEQ ID NO:52 at251 primer sequence
SEQ ID NO:53 at252 primer sequence
SEQ ID NO:54 at257 primer sequence
SEQ ID NO:55 at258 primer sequence
SEQ ID NO:56 at259 primer sequence
SEQ ID NO:57 at260 primer sequence
SEQ ID NO:58 at270 primer sequence
SEQ ID NO:59 at271 primer sequence
SEQ ID NO:60 at272 primer sequence
SEQ ID NO:61 at273 primer sequence
SEQ ID NO:62 Nucleotide sequence of the FAD2 gene of Y. lipolytica strain W29 including upstream and downstream regions. Nucleotides 1-1,000 correspond to the 5′ upstream sequence, nucleotides 1,001-2,260 correspond to the protein coding region for the A12 desaturase, and nucleotides 2,261-3,260 correspond to the 3′ downstream region.
SEQ ID NO:63 Nucleotide sequence of hygromycin resistance selectable marker gene (pTEF-Hyg-tLip2). Nucleotides 1-417 correspond to the TEF promoter (Muller et al., 1998; Accession No. AF054508), nucleotides 418-1,443 correspond to the protein coding region for the hygromycin phosphotransferase (Hph) enzyme, and nucleotides 1,444-1,620 correspond to the polyadenylation region/transcription terminator from the Y. lipolytica strain U6 lipase 2 gene, from Accession No. HM486900 (Darvishi et al., 2011); 1,620nt.
SEQ ID NO:64 Amino acid sequence of hygromycin B phosphotransferase (Hph) encoded by pTEF-Hyg-tLip2
SEQ ID NO:65 Nucleotide sequence of the nourseothricin resistance selectable marker gene (pTEF-Nat1-tLip2); Accession No. AIC06992, Laroude et al. (2019); Nucleotides 1-418 correspond to the TEF promoter, nucleotides 419-988 correspond to the protein coding region for the nourseothricin acetyltransferase (Nat1) enzyme, and nucleotides 989-1,165 correspond to the polyadenylation region/transcription terminator from the Lip2 gene; 1,165nt.
SEQ ID NO:66 Amino acid sequence of nourseothricin acetyltransferase (Nat1) encoded by the pTEF-Nat1-tLip2 gene.
SEQ ID NO:67 Amino acid sequence of Y. lipolytica strain URA3 polypeptide, GenBank Accession No. Q12724; 286aa.
SEQ ID NO:68 Nucleotide sequence of a URA3 gene of Y. lipolytica including upstream and downstream regions. Nucleotides 1-1,000 correspond to the 5′ upstream sequence, nucleotides 1,001-1,861 correspond to the protein coding region for the orotidine-5′-phosphate decarboxylase, and nucleotides 1,862-2,861 correspond to the 3′ downstream region.
SEQ ID NO:69 Nucleotide sequence of the DGA1 gene (YALI0E32769p) of Y. lipolytica strain W29, chromosome E, nucleotides 3885857 to 3889401 of Accession No. CR382131.1, including upstream and downstream regions of the DGA1 gene. Nucleotides 1-1,000 correspond to the 5′ upstream sequence, nucleotides 1,001-2,545 correspond to the protein coding region for the DGAT1, and nucleotides 2,546-3,545 correspond to the 3′ downstream region; 3,545nt.
SEQ ID NO:70 Amino acid sequence of DGAT1 from Y. lipolytica strain W29, encoded by the YALI0E32769p gene, Genbank Accession No. XP_504700.1; 514aa.
SEQ ID NO:71 Nucleotide sequence of the DGA2 gene (YALI0D07986p) of Y. lipolytica strain W29, chromosome D, nucleotides 1025413 to 1028993 of Accession No. CP017556.1, including upstream and downstream regions of the DGA2 gene. Nucleotides 1-1,000 correspond to the 5′ upstream sequence, nucleotides 1,001-2,581 correspond to the protein coding region for the DGAT2, and nucleotides 2,582-3,581 correspond to the 3′ downstream region; 3,581nt.
SEQ ID NO:72 Amino acid sequence of Y. lipolytica strain W29 DGAT2, Genbank Accession No. XP_502557; 526aa.
SEQ ID NO:73 Nucleotide sequence of the LRO1 gene (YALI0E16797p) of Y. lipolytica strain CLIB122, chromosome E, nucleotides 1989950 to 1993896 of Accession No. CR382131.1, including upstream and downstream regions of the LRO1 gene. Nucleotides 1-1,000 correspond to the 5′ upstream sequence, nucleotides 1,001-2,947 correspond to the protein coding region for the PDAT, and nucleotides 2,948-3,947 correspond to the 3′ downstream region; 3,947nt.
SEQ ID NO:74 Amino acid sequence of PDAT from Y. lipolytica strain CLIB122, encoded by the LRO1 gene (YALI0E16797p), Genbank Accession No. XP_504038; 648aa.
SEQ ID NO:75 Nucleotide sequence of the ARE1 gene (YALI0F06578p) of Y. lipolytica strain W29, chromosome F, nucleotides 957751 to 961382 of Accession No. CP028453.1, including upstream and downstream regions of the ARE1 gene. Nucleotides 1-1,000 correspond to the 5′ upstream sequence, nucleotides 1,001-2,632 correspond to the protein coding region for the ASAT, and nucleotides 2,633-3,632 correspond to the 3′ downstream region; 3,632.
SEQ ID NO:76 Amino acid sequence of ASAT from Y. lipolytica strain W29, encoded by the ARE1 gene (YALI0F06578p), GenBank Accession No. XP_505086; 543aa.
SEQ ID NO:77 Nucleotide sequence of the POX2 gene (YALI0F10857g) of Y. lipolytica strain W29 including upstream and downstream regions. Nucleotides 1-1,000 correspond to the 5′ upstream sequence, nucleotides 1,001-3,103 correspond to the protein coding region for the acyl-CoA oxidase, and nucleotides 3,104-4,103 correspond to the 3′ downstream region.
SEQ ID NO:78 Amino acid sequence of the POX2 gene product (Accession No. XP_505264.1) of Y. lipolytica strain CLIB122; 700aa.
SEQ ID NO:79 Nucleotide sequence of the POX1 gene (YGL205W; chrVII:108158-110404) of S. cerevisiae including upstream and downstream regions. Nucleotides 1-1,000 correspond to the 5′ upstream sequence, nucleotides 1,001-3,247 correspond to the protein coding region for the acyl-CoA oxidase, and nucleotides 3,248-4,247 correspond to the 3′ downstream region.
SEQ ID NO:80 Amino acid sequence of the POX1 gene product (Accession No. NP_011310.1) of S. cerevisiae strain S288C; 748aa.
SEQ ID NO:81 Nucleotide sequence of the promoter of the PGK1 gene of S. cerevisiae strain S288c, chromosome III, Accession No. CP020125.1). The translation start ATG is nucleotides 586-588; 588nt.
SEQ ID NO:82 Nucleotide sequence of the promoter of the ENO1 gene of S. cerevisiae strain S288c, chromosome III, (Uemura et al., 1986; Accession No. D14474.1). The translation start ATG is nucleotides 518-520; 520nt.
SEQ ID NO:83 Nucleotide sequence of the promoter of the TDH3 gene of S. cerevisiae, (Behall et al., 1989; Accession No. M28222.1). The translation start ATG is nucleotides 668-670; 670nt.
SEQ ID NO:84 Nucleotide sequence of the transcription terminator/polyadenylation region of the PDK gene of S. cerevisiae; 278nt.
SEQ ID NO:85 Nucleotide sequence of the transcription terminator/polyadenylation region of the CYC1 gene of S. cerevisiae; 282nt.
SEQ ID NO:86 Nucleotide sequence of the transcription terminator/polyadenylation region of the ENOI gene of S. cerevisiae; 288nt.
SEQ ID NO:87 Nucleotide sequence of the POX1 gene (YALI0E32835g) of Y. lipolytica strain CLIB122, chromosome E, nucleotides 3897102 to 3899135 of Accession No. CR382131.1, including upstream and downstream regions of the POX1 gene. Nucleotides 1-1,000 correspond to the 5′ upstream sequence, nucleotides 1,001-3,103 correspond to the protein coding region for the POX1, and nucleotides 3,104-4,103 correspond to the 3′ downstream region; 4,103 nt.
SEQ ID NO:88 Amino acid sequence of POX1 from Y. lipolytica strain CLIB122, encoded by YALI0E32835p, GenBank Accession No. XP_504703.1; 677 aa.
SEQ ID NO:89 Nucleotide sequence of the POX3 gene (YALI0D24750g) of Y. lipolytica strain CLIB122, chromosome D, nucleotides 3291579 to 3293681 of Accession No. CR382130.1, including upstream and downstream regions of the POX3 gene. Nucleotides 1-1,000 correspond to the 5′ upstream sequence, nucleotides 1,001-3,103 correspond to the protein coding region for the POX3, and nucleotides 3,104-4,103 correspond to the 3′ downstream region; 4,103 nt.
SEQ ID NO:90 Amino acid sequence of Y. lipolytica strain CLIB122 POX3, encoded by YALI0D24750p, GenBank Accession No. XP_503244; 700 aa.
SEQ ID NO:91 Nucleotide sequence of the MFE1 gene (YALI0E15378g) of Y. lipolytica strain CLIB122, chromosome E, nucleotides 1829460 to 1832239 of Accession No. CR382131.1, including upstream and downstream regions of the MFE1 gene. Nucleotides 1-1,000 correspond to the 5′ upstream sequence, nucleotides 1,001-3,706 correspond to the protein coding region for the PDAT, and nucleotides 3,706-4,706 correspond to the 3′ downstream region; 4,706 nt.
SEQ ID NO:92 Amino acid sequence of MFE from Y. lipolytica strain CLIB122, encoded by YALI0E15378p, GenBank Accession No. XP_503980; 901 aa.
SEQ ID NO:93 Nucleotide sequence of the PEX10 gene (YALI0C01023g) of Y. lipolytica strain CLIB122, chromosome C, nucleotides 139718 to 140851 of Accession No. CR382129.1, including upstream and downstream regions of the PEX10 gene. Nucleotides 1-1,000 correspond to the 5′ upstream sequence, nucleotides 1,001-2,134 correspond to the protein coding region for the PEX10, and nucleotides 2,135-3,134 correspond to the 3′ downstream region; 3,134.
SEQ ID NO:94 Amino acid sequence of PEX10 from Y. lipolytica strain CLIB122, encoded by YALI0C01023p, GenBank Accession No. XP_501311; 377 aa.
SEQ ID NO:95 Nucleotide sequence of the PLB1 gene (YALI0E16060g) of Y. lipolytica strain CLIB122, chromosome E, nucleotides 1913947 to 1915863 of Accession No. CR382131.1, including upstream and downstream regions of the PLB1 gene. Nucleotides 1-1,000 correspond to the 5′ upstream sequence, nucleotides 1,001-2,917 correspond to the protein coding region for the PLB1, and nucleotides 2,918-3,917 correspond to the 3′ downstream region; 3,917 nt.
SEQ ID NO:96 Amino acid sequence of PLB1 from Y. lipolytica strain CLIB122, encoded by YALI0E16060p, GenBank Accession No. XP_504006; 638 aa.
SEQ ID NO:97 Nucleotide sequence of the SNF1 gene (YALI0D02101g) of Y. lipolytica strain CLIB122, chromosome D, nucleotides 236133 to 237872 of Accession No. CR382130.1, including upstream and downstream regions of the SNF1 gene. Nucleotides 1-1,000 correspond to the 5′ upstream sequence, nucleotides 1,001-2,740 correspond to the protein coding region for the SNF1, and nucleotides 2,741-3,740 correspond to the 3′ downstream region; 3,740 nt.
SEQ ID NO:98 Amino acid sequence of SNF1 from Y. lipolytica strain CLIB122, encoded by YALI0D02101p, GenBank Accession No. XP_502312; 579 aa.
SEQ ID NO:99 Nucleotide sequence of the SPO14 gene (YALI0E18898g) of Y. lipolytica strain CLIB122, chromosome E, nucleotides 2251884 to 2257373 of Accession No. CR382131.1, including upstream and downstream regions of the SPO14 gene. Nucleotides 1-1,000 correspond to the 5′ upstream sequence, nucleotides 1,001-6,490 correspond to the protein coding region for the SPO14, and nucleotides 6,491-7,490 correspond to the 3′ downstream region; 7,490 nt.
SEQ ID NO:100 Amino acid sequence of SPO14 from Y. lipolytica strain CLIB122, encoded by YALI0E18898p, GenBank Accession No. XP_504124; 1829 aa.
SEQ ID NO:101 Nucleotide sequence of the OPI1 gene (YALI0C14784g) of Y. lipolytica strain CLIB122, chromosome E, nucleotides 2251884 to 237872 of Accession No. CR382129.1, including upstream and downstream regions of the OPI1 gene. Nucleotides 1-1,000 correspond to the 5′ upstream sequence, nucleotides 1,001-2,863 correspond to the protein coding region for the OPI1, and nucleotides 2,864-3,863 correspond to the 3′ downstream region; 3,863 nt.
SEQ ID NO:102 Amino acid sequence of OPI1 from Y. lipolytica strain CLIB122, encoded by YALI0C14784p, GenBank Accession No. XP_501843; 620 aa.
SEQ ID NO: 103 Nucleotide sequence of a portion of the ITS of Mortierella alpina strain ATCC 32222; 178nt.
SEQ ID NO: 104 Nucleotide sequence of ITS of Mucor hiemalis 14183 isolate 1, 640nt.
SEQ ID NO: 105 Nucleotide sequence of ITS of M. alpina 14183 isolate 2, designated strain yNI0133; 669nt.
SEQ ID NO: 106 Nucleotide sequence of ITS of M. alpina 14183 isolate 3, designated strain yNI0134, 671nt.
SEQ ID NO: 107 Nucleotide sequence of ITS of M. alpina 14183 isolate 4, designated strain yNI0135, 672nt.
SEQ ID NO: 108 Nucleotide sequence of ITS of M. alpina 14183 isolate 21, 668nt.
SEQ ID NO: 109 Nucleotide sequence of ITS of M. alpina 14183 isolate 22, 671nt.
SEQ ID NO: 110 Nucleotide sequence of ITS of M. alpina 14183 isolate 23, 670nt.
SEQ ID NO: 111 Nucleotide sequence of ITS of 14183 isolate 24, possibly Trichoderma asperellum; 824nt.
SEQ ID NO: 112 Nucleotide sequence of ITS of M. alpina 14183 isolate 25, 668nt.
SEQ ID NO: 113 Nucleotide sequence of ITS of Mucor hiemalis Namadji I isolate 1, designated yNI0121; 640nt.
SEQ ID NO: 114 Nucleotide sequence of ITS of Mucor hiemalis Namadji I isolate 3, designated yNI0122; 639nt.
SEQ ID NO: 115 Nucleotide sequence of ITS of Mucor hiemalis Namadji I isolate 4, designated yNI0124; 647nt.
SEQ ID NO: 116 Nucleotide sequence of ITS of Mucor hiemalis Namadji I isolate 5, designated yNI0123; 639nt.
SEQ ID NO: 117 Nucleotide sequence of ITS of Mucor hiemalis Namadji I isolate 6; 640nt.
SEQ ID NO: 118 Nucleotide sequence of ITS of Mucor hiemalis Namadji I isolate 8; 639nt.
SEQ ID NO: 119 Nucleotide sequence of ITS of Mucor hiemalis Namadji I isolate 9; 646nt.
SEQ ID NO: 120 Nucleotide sequence of ITS of Mucor hiemalis Namadji I isolate 10; 640nt.
SEQ ID NO: 121 Nucleotide sequence of ITS of Mortierella elongata Namadji I isolate 11; 659nt.
SEQ ID NO: 122 Nucleotide sequence of ITS of Mucor hiemalis Namadji I isolate 12; 639nt.
SEQ ID NO: 123 Nucleotide sequence of ITS of Mucor hiemalis Namadji I isolate 14; 640nt.
SEQ ID NO: 124 Nucleotide sequence of ITS of Mucor hiemalis Namadji I isolate 15; 639nt.
SEQ ID NO: 125 Nucleotide sequence of ITS of Mucor hiemalis Namadji I isolate 21; 639nt.
SEQ ID NO: 126 Nucleotide sequence of ITS of Mortierella sp. Namadji II isolate 1, designated yNI0126; 637nt.
SEQ ID NO: 127 Nucleotide sequence of ITS of Mortierella sp. Namadji II isolate 2, designated yNI0127; 640nt.
SEQ ID NO: 128 Nucleotide sequence of ITS of Mortierella sp. Namadji II isolate 3, designated yNI0128; 629nt.
SEQ ID NO: 129 Nucleotide sequence of ITS of Mortierella sp. Namadji II isolate 4, designated yNI0129; 640nt.
SEQ ID NO: 130 Nucleotide sequence of ITS of Mortierella sp. Namadji II isolate 5, designated yNI0130; 640nt.
SEQ ID NO: 131 Nucleotide sequence of ITS of Mortierella sp. Namadji II isolate 6; 630nt.
SEQ ID NO: 132 Nucleotide sequence of ITS of Mortierella sp. Namadji II isolate 7; 636nt.
SEQ ID NO: 133 Nucleotide sequence of ITS of Mortierella sp. Namadji II isolate 8; 630nt.
SEQ ID NO: 134 Nucleotide sequence of ITS of Mortierella elongata Namadji II isolate 9, designated yNI0131; 640nt.
SEQ ID NO: 135. Nucleotide sequence of ITS of Mortierella sp. Namadji II isolate 10; 652nt.
SEQ ID NO: 136 Nucleotide sequence of ITS of Mortierella sp. Namadji II isolate 11; 633nt.
SEQ ID NO: 137 Nucleotide sequence of ITS of Mortierella sp. Namadji II isolate 12; 639nt.
SEQ ID NO: 138. Nucleotide sequence of ITS of Mortierella sp. Namadji II isolate 13; 638nt.
SEQ ID NO: 139 Nucleotide sequence of ITS of Mortierella sp. Namadji II isolate 14; 640nt.
SEQ ID NO: 140 Nucleotide sequence of ITS of Mortierella sp. Namadji II isolate 15; 640nt.
SEQ ID NO: 141 Nucleotide sequence of ITS of Mortierella sp. Namadji II isolate 16; 641nt.
SEQ ID NO: 142 Nucleotide sequence of ITS of Mortierella sp. Namadji II isolate 17; 640nt.
SEQ ID NO: 143 Nucleotide sequence of ITS of Mortierella sp. Namadji II isolate 18; 640nt.
SEQ ID NO: 144 Nucleotide sequence of ITS of Mortierella sp. Namadji II isolate 19; 643nt.
SEQ ID NO: 145 Nucleotide sequence of ITS of Mortierella sp. Namadji II isolate 20; 629nt.
SEQ ID NO: 146 Nucleotide sequence of ITS of Mortierella sp. Namadji II isolate 21; 628nt.
SEQ ID NO: 147 Nucleotide sequence of oligonucleotide primer xMaF1; 22nt.
SEQ ID NO: 148 Nucleotide sequence of oligonucleotide primer xMaF2; 19nt.

### DETAILED DESCRIPTION OF THE INVENTION

### General Techniques and Standard Definitions

Unless specifically defined otherwise, all technical and scientific terms used herein shall be taken to have the same meaning as commonly understood by one of ordinary skill in the art 5 (e.g., in cell culture, fermentation, molecular genetics, protein chemistry, non-meat food products and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term about, unless stated to the contrary, refers to +/−20%, more preferably +/−10%, more preferably +/−5%, more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

As used herein, a “lipid” is any of a class of organic compounds that are or comprise fatty acids, which may be esterified or non-esterified, or their derivatives and are insoluble in water but soluble in organic solvents, for example in chloroform. As used herein, the term “extracted lipid” refers to a lipid composition which has been extracted from a microbial cell. The extracted lipid can be a relatively crude composition obtained by, for example, lysing the cells, or a more purified composition where most, if not all, of one or more or each of the water, nucleic acids, proteins and carbohydrates derived from the cells have been removed. Examples of purification methods are described below. In an embodiment, the extracted lipid comprises at least about 10%, at least about 20%, at least about 30%, at least about 40%, at least about 50%, at least about 60%, at least about 70%, at least about 80%, at least about 90%, or at least about 95% (w/w) lipid by weight of the composition. In embodiments, the extracted lipid comprises between about 10% and 95% lipid by weight, for example between about 10% and about 50%, or about 50% and 95%, lipid by weight. The lipid may be solid or liquid at room temperature (25° C.), or a mixture of the two; when liquid it is considered to be an oil, when solid it is considered to be a fat. In an embodiment, extracted lipid of the invention has not been blended with another lipid produced from another source, for example, animal lipid. Alternatively, the extracted lipid may be blended with a different lipid.

As used herein, the term “polar lipid” refers to amphipathic lipid molecules having a hydrophilic head and a hydrophobic tail, including phospholipids (e.g. phosphatidylcholine, phosphatidylethanolamine, phosphatidylinositol, phosphatidylserine, phosphatidylglycerol, diphosphatidylglycerols), cephalins, sphingolipids (sphingomyelins and glycosphingolipids), phosphatidic acid, cardiolipin and glycoglycerolipids. Phospholipids are composed of the following major structural units: fatty acids, glycerol, phosphoric acid, and amino alcohols. They are generally considered to be structural lipids, playing important roles in the structure of the membranes of plants, microbes and animals. Because of their chemical structure, polar lipids exhibit a bipolar nature, exhibiting solubility or partial solubility in both polar and non-polar solvents.

The term “phospholipid”, as used herein, refers to an amphipathic molecule, having a hydrophilic head and a hydrophobic tail, that has a glycerol backbone esterified to a phosphate “head” group and two fatty acids which provide the hydrophobic tail. The phosphate group can be modified with simple organic molecules such as choline, ethanolamine or serine. Due to their charged headgroup at neutral pH, phospholipids are polar lipids, having some solubility in solvents such as ethanol in addition to solvents such as chloroform. Phospholipids are a key component of all cell membranes. They can form lipid bilayers because of their amphiphilic characteristic. Well known phospholipids include phosphatidylcholine (PC), phosphatidylethanolamine (PE), phosphatidylinositol (PI), phosphatidylserine (PS), phosphatidic acid (PA), phosphatidylglycerol (PG) and cardiolipin.

As used herein, the term “non-polar lipid” refers to fatty acids and derivatives thereof which are soluble in organic solvents but insoluble in water. The fatty acids may be free fatty acids and/or in an esterified form. Examples of esterified forms include, but are not limited to, triacylglycerol (TAG), diacylyglycerol (DAG), monoacylglycerol (MAG). Non-polar lipids also include sterols, sterol esters and wax esters. Non-polar lipids are also known as “neutral lipids” or in some contexts referred to as “oils”. Non-polar lipid may be a liquid at room temperature, or a solid, depending on the degree of unsaturation of the fatty acids in the non-polar lipid. Typically, the more saturated the fatty acid content, the higher the melting temperature of the lipid.

As used herein, the term “fatty acid” refers to a carboxylic acid consisting of an aliphatic hydrocarbon chain and a terminal carboxyl group. The hydrocarbon chain can be either saturated or unsaturated. Unsaturated fatty acids include monounsaturated fatty acids having only one carbon-carbon double bond and polyunsaturated fatty acids (PUFA) having at least two carbon-carbon double bonds, typically between 2 and 6 carbon-carbon double bonds. A fatty acid may be a free fatty acid (FFA) or esterified to a glycerol or glycerol-phosphate molecule, CoA molecule or other headgroup as known in the art, preferably esterified as part of a polar lipid such as a phospholipid.

As used herein, the term “total fatty acid (TFA) content” or variations thereof refers to the total amount of fatty acids in, for example, the extracted lipid or cell, on a weight basis. The TFA may be expressed as a percentage of the weight of the cell or other fraction, e.g., as a percentage of the polar lipid. Unless otherwise specified, the weight with regard to the cell weight is the dry cell weight (DCW). In an embodiment, TFA content is measured by conversion of the fatty acids to fatty acid methyl esters (FAME) or fatty acid butyl esters (FABE) and measurement of the amount of FAME or FABE by GC, using addition of a known amount of a distinctive fatty acid standard as a quantitation standard in the GC. Typically, the amount and fatty acid composition of lipids comprising only fatty acids in the range of C10-C24 are determined by conversion to FAME, whereas lipids comprising fatty acids in the range of C4-C10 are determined by conversion to FABE. TFA therefore represents the weight of just the fatty acids, not the weight of the fatty acids and their linked moieties in the lipid.

“Saturated fatty acids” do not contain any double bonds or other functional groups along the acyl chain. The term “saturated” refers to hydrogen, in that all carbons (apart from the carboxylic acid [—COOH] group) contain as many hydrogens as possible.

“Unsaturated fatty acids” are of similar form to saturated fatty acids, except that one or more alkene functional groups exist along the chain, with each alkene substituting a singly-bonded “—CH2-CH2-” part of the chain with a doubly-bonded “—CH═CH—” portion (that is, a carbon double bonded to another carbon). The two next carbon atoms in the chain that are bound to either side of the double bond can occur in a cis or trans configuration, preferably in the cis configuration.

As used herein, the term “monounsaturated fatty acid” refers to a fatty acid which comprises at least 12 carbon atoms in its carbon chain and only one alkene group (carbon-carbon double bond) in the chain. Monounsaturated fatty acids include C12:1Δ9, C14:1Δ9, C16:1Δ9 (palmitoleic acid), C18:1Δ9 (oleic acid) and C18:1Δ11 (vaccenic acid).

As used herein, the terms “polyunsaturated fatty acid” or “PUFA” refer to a fatty acid which comprises at least 12 carbon atoms in its carbon chain and at least two alkene groups (carbon-carbon double bonds). Ordinarily, the number of carbon atoms in the carbon chain of the fatty acids refers to an unbranched carbon chain. Unless stated otherwise, if the carbon chain is branched, the number of carbon atoms excludes those in side groups. Polar lipids of the invention, such as in an extract or cell of the invention, comprise at least one ω6 fatty acid having a desaturation (carbon-carbon double bond) in the sixth carbon-carbon bond from the methyl end of the fatty acid. Examples of ω6 fatty acid include, but are not limited to, arachidonic acid (ARA, C20:4Δ5,8,11,14; ω6), dihomo-γ-linolenic acid (DGLA, C20:3Δ8,11,14; ω6), eicosadienoic acid (EDA, C20:2Δ11,14; ω6), docosatetraenoic acid (DTA, C22:4Δ7,10,13,16; ω6), docosapentaenoic acid-ω6 (DPA-ω6, C22:5Δ4,7,10,13,16; ω6), γ-linolenic acid (GLA, C18:3Δ6,9,12; ω6) and linoleic acid (LA, C18:2Δ9,12; ω6). In some embodiments, polar lipid of the invention, such as in an extract or cell of the invention, comprise at least one ω3 fatty acid having a desaturation (carbon-carbon double bond) in the third carbon-carbon bond from the methyl end of the fatty acid. In some embodiments, polar lipid of the invention, such as in an extract or cell of the invention, does not comprise specific ω3 fatty acids such as one or more of C16:3ω3, ALA, EPA and DHA, or does not comprise any ω3 fatty acids. Examples of ω3 fatty acids include, but are not limited to, α-linolenic acid (ALA, C18:3Δ9,12,15; ω3), hexadecatrienoic acid (C16:3ω3), eicosapentaenoic acid (EPA, C20:5Δ5,8,11,14,17; ω3), docosapentaenoic acid (DPA, C22:5Δ7,10,13,16,19, ω3), docosahexaenoic acid (DHA, 22:6Δ4,7,10,13,16,19, ω3), eicosatetraenoic acid (ETA, C20:4Δ8,11,14,17; ω3) and eicosatrienoic acid (ETrA, C20:3Δ11,14,17; ω3). In some embodiments, polar lipid of the invention, such as in an extract or cell of the invention, does not comprise one or more or all of the following ω3 fatty acids; C16:3ω3, EPA and DHA.

As used herein, “C12:0” refers to lauric acid.

As used herein, “C14:0” refers to myristic acid.

As used herein, “C15:0” refers to n-pentadecanoic acid.

As used herein, “C16:0” refers to palmitic acid.

As used herein, “C17:1” refers to heptadecenoic acid.

As used herein, “C16:1Δ9” refers to palmitoleic acid, or-hexadec-9-enoic acid.

As used herein, “C18:0” refers to stearic acid.

As used herein, “C18:1Δ9”, sometimes referred to in shorthand as “C18:1”, refers to oleic acid.

As used herein, “C18:1Δ11” refers to vaccenic acid.

As used herein, “C20:0” refers to eicosanoic acid.

As used herein, “C20:1” refers to eicosenoic acid.

As used herein, “C22:0” refers to docosanoic acid.

As used herein, “C22:1” refers to erucic acid.

As used herein, “C24:0” refers to tetracosanoic acid.

“Triacylglyceride”, “triacylglycerol” or “TAG” is a glyceride in which the glycerol is esterified with three fatty acids which may be the same (e.g. as in tri-olein) or, more commonly, different. All three of the fatty acids may be different, or two of the fatty acids may be the same and the third is different. In the Kennedy pathway of TAG synthesis, DAG is formed as described below, and then a third acyl group is esterified to the glycerol backbone by the activity of a diglyceride acyltransferase (DGAT). TAG is a form of non-polar lipid. The three acyl groups esterified in a TAG molecule are referred to as being esterified in the sn-1, sn-2 and sn-3 positions, referring to the positions in the glycerol backbone of the TAG molecule. The sn-1 and sn-3 positions are chemically identical, but biochemically the acyl groups esterified in the sn-1 and sn-3 positions are distinct in that separate and distinct acyltransferase enzymes catalyse the esterifications.

“Diacylglyceride”, “diacylglycerol” or “DAG” is glyceride in which the glycerol is esterified with two fatty acids which may be the same or, preferably, different. As used herein, DAG comprises a hydroxyl group at a sn-1,3 or sn-2 position, and therefore DAG does not include phosphorylated glycerolipid molecules such as PA or PC. In the Kennedy pathway of DAG synthesis, the precursor sn-glycerol-3-phosphate (G3P) is esterified to two acyl groups, each coming from a fatty acid coenzyme A ester, in a first reaction catalysed by a glycerol-3-phosphate acyltransferase (GPAT) at position sn-1 to form LysoPA, followed by a second acylation at position sn-2 catalysed by a lysophosphatidic acid acyltransferase (LPAAT) to form phosphatidic acid (PA). This intermediate is then de-phosphorylated by PAP to form DAG.

As used herein, an “oil” is a composition comprising predominantly lipid and which is a liquid at room temperature.

As used herein, an “oleaginous” cell or microbe is one that is capable of storing at least 20% lipid, such as for example 20% to 70%, of its cell mass on a dry weight basis. The lipid content may depend on culture conditions, as is known in the art. It is understood that so long as the microbe is capable of synthesizing and accumulating at least 20% lipid on a dry cell weight basis under at least one set of culture conditions it is regarded as an oleaginous cell, even if under different conditions it accumulates less than 20% lipid. As used herein, a “microbe which is derived from an oleaginous microbe” is a microbe which is derived from a progenitor oleaginous microbe by one or more genetic modifications. The microbe which is derived from an oleaginous microbe may itself be an oleaginous microbe, or it may produce less than 20% lipid and not be an oleaginous microbe. The genetic modifications may have been introduced by human intervention or be naturally occurring, so long as at least one of the genetic modifications was introduced by human intervention. In an embodiment, the genetic modifications to produce the derived microbe comprise one or more genetic modifications which result in a reduced synthesis and/or accumulation of TAG.

As used herein, a “heterotrophic” cell is one that is capable of utilizing organic materials as a carbon source for metabolism and growth. Heterotrophic organisms may also be able to grow autotrophically under suitable conditions.

As used herein, “fermentation” refers to a metabolic process that produces chemical changes in organic substrates through the action of enzymes in the cells, under conditions either lacking oxygen or having reduced levels of oxygen relative to air.

As used herein, a “meat-like flavour and/or aroma”, or a “meat-associated flavour and/or aroma” refers to flavours and/or aromas that are the same as or are similar to one or more meats, such as beef, steak, chicken, for example roasted chicken or chicken skin, pork, lamb, duck, venison, chicken or other meat soup, meat broth or liver. Such aromas are typically detected by human volunteers, for example by a qualified sensory panel. Meat-like or meat-associated flavours and/or aromas can also be detected by assessing volatile compounds arising after the cooking of the composition or food. Volatile compounds indicative of meat-like or meat-associated aromas and flavours are known in the art and include those exemplified herein, including but not limited to 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, and 1,3,5-thitriane.

### Microbial Lipids

Provided are microbial lipids, and in particular extracted microbial lipids, which are suitable for use in compositions, foods, feedstuffs and beverages for imparting meat-like aromas and/or flavours to the compositions, foods, feedstuffs and beverages when those compositions, foods, feedstuffs and beverages are heated.

In one aspect, provided is an extracted microbial lipid, comprising esterified fatty acids in the form of either (i) polar lipid without any non-polar lipid, or (ii) polar lipid and non-polar lipid, the polar lipid preferably being present in the extracted microbial lipid in a greater amount than the non-polar lipid, wherein
(a) the polar lipid of (i) and (ii) comprises a total fatty acid (TFA) content which comprises the ω6 fatty acids, wherein at least some of the ω6 fatty acids are esterified in the form of phospholipids in the polar lipid, and wherein the ω6 fatty acids comprise two, three, four or more fatty acids selected from the group consisting of arachidonic acid (ARA), dihomo-γ-linolenic acid (DGLA), eicosadienoic acid (EDA), docosatetraenoic acid (DTA), docosapentaenoic acid-ω6 (DPA-ω6) and γ-linolenic acid (GLA),
(b) the phospholipids in the polar lipid comprise at least two, preferably three or all four, of phosphatidylcholine (PC), phosphatidylethanolamine (PE), phosphatidylinositol (PI) and phosphatidylserine (PS), each comprising one or more of ARA, DGLA, EDA, DTA, DPA-ω6 and GLA, and optionally one or more of phosphatidic acid (PA), phosphatidylglycerol (PG) and cardiolipin (Car), each comprising one or more of ARA, DGLA, EDA, DTA, DPA-ω6 and GLA,
(c) the polar lipid comprises a total saturated fatty acid content comprising palmitic acid and stearic acid,
(d) the polar lipid comprises a total monounsaturated fatty acid content comprising oleic acid and palmitoleic acid (C16:1Δ9cis), and
(e) ω3 fatty acids are either absent from the polar lipid or are present in a total amount of less than about 3% by weight of the TFA content of the polar lipid, and/or wherein the polar lipid lacks C16:2, C16:3ω3, EPA and DHA.

In another aspect, provided is an extracted microbial lipid, comprising esterified fatty acids in the form of either (i) polar lipid without any non-polar lipid, or (ii) polar lipid and non-polar lipid, the polar lipid preferably being present in the extracted microbial lipid in a greater amount than the non-polar lipid, wherein
(a) the polar lipid of (i) and (ii) comprises a total fatty acid (TFA) content which comprises ω6 fatty acids, wherein at least some of the ω6 fatty acids are esterified in the form of phospholipids in the polar lipid, the ω6 fatty acids comprising arachidonic acid (ARA), dihomo-γ-linolenic acid (DGLA), eicosadienoic acid (EDA), docosatetraenoic acid (DTA), docosapentaenoic acid-ω6 (DPA-ω6) or γ-linolenic acid (GLA), or any combination thereof,
(b) the phospholipids in the polar lipid comprise phosphatidylcholine (PC), phosphatidylethanolamine (PE), phosphatidylinositol (PI) and phosphatidylserine (PS), each comprising one or more of ARA, DGLA, EDA, DTA, DPA-ω6 and GLA, and optionally one or more of phosphatidic acid (PA), phosphatidylglycerol (PG) and cardiolipin (Car), each comprising one or more of ARA, DGLA, EDA, DTA, DPA-ω6 and GLA,
(c) the polar lipid comprises a total saturated fatty acid content comprising palmitic acid and stearic acid, and
(d) the polar lipid comprises a total monounsaturated fatty acid content comprising oleic acid and palmitoleic acid (C16:1Δ9cis).

In another aspect, the present invention provides an extracted microbial lipid, comprising esterified fatty acids in the form of either (i) polar lipid without any non-polar lipid, or (ii) polar lipid and non-polar lipid, the polar lipid preferably being present in the extracted microbial lipid in a greater amount than the non-polar lipid, wherein
(a) the polar lipid of (i) and (ii) comprises a total fatty acid (TFA) content which comprises ω6 fatty acids, wherein at least some of the ω6 fatty acids are esterified in the form of phospholipids in the polar lipid, the ω6 fatty acids comprising arachidonic acid (ARA), dihomo-γ-linolenic acid (DGLA), eicosadienoic acid (EDA), docosatetraenoic acid (DTA), docosapentaenoic acid-ω6 (DPA-ω6) or γ-linolenic acid (GLA), or any combination thereof,
(b) the polar lipid comprises a total saturated fatty acid content comprising palmitic acid and stearic acid,
(c) the polar lipid comprises a total monounsaturated fatty acid content comprising oleic acid and palmitoleic acid (C16:1Δ9cis),
(d) ω3 fatty acids are either absent from the polar lipid or are present in a total amount of less than about 3% by weight of the TFA content of the polar lipid, and/or wherein the polar lipid lacks C16:2, C16:3 ω3, EPA and DHA.

In another aspect, the present invention provides an extracted microbial lipid, comprising ω6 fatty acids esterified in the form of polar lipid, wherein
(a) the polar lipid comprises a total fatty acid (TFA) content which comprises ω6 fatty acids, wherein at least some of the ω6 fatty acids are esterified in the form of phospholipids in the polar lipid, the ω6 fatty acids comprising arachidonic acid (ARA), dihomo-γ-linolenic acid (DGLA), eicosadienoic acid (EDA), docosatetraenoic acid (DTA), docosapentaenoic acid-ω6 (DPA-ω6) or γ-linolenic acid (GLA), or any combination thereof,
(b) the phospholipids in the polar lipid comprise phosphatidylcholine (PC), phosphatidylethanolamine (PE), phosphatidylinositol (PI) and phosphatidylserine (PS), each comprising one or more of ARA, DGLA, EDA, DTA, DPA-ω6 and GLA, and optionally one or more of phosphatidic acid (PA), phosphatidylglycerol (PG) and cardiolipin (Car), each comprising one or more of ARA, DGLA, EDA, DTA, DPA-ω6 and GLA,
(c) the polar lipid comprises a total saturated fatty acid content comprising palmitic acid and stearic acid, and
(d) the polar lipid comprises a total monounsaturated fatty acid content comprising oleic acid and palmitoleic acid (C16:1Δ9cis).

In another aspect, the present invention provides an extracted microbial lipid comprising ω6 fatty acids esterified in the form of polar lipid, wherein
(a) the polar lipid comprises a total fatty acid (TFA) content which comprises the ω6 fatty acids, wherein at least some of the ω6 fatty acids are esterified in the form of phospholipids in the polar lipid, and wherein the ω6 fatty acids comprise one or two or all three of eicosadienoic acid (EDA), docosatetraenoic acid (DTA) and docosapentaenoic acid-ω6 (DPA-ω6),
(b) γ-linolenic acid (GLA) is either absent from the polar lipid or is present in the polar lipid,
(c) the polar lipid comprises a total saturated fatty acid content comprising palmitic acid and stearic acid, and
(d) the polar lipid comprises a total monounsaturated fatty acid content comprising oleic acid and palmitoleic acid (C16:1Δ9cis).

In another aspect, provided is an extracted microbial lipid comprising ω6 fatty acids esterified in the form of polar lipid, wherein
(a) the polar lipid comprises a total fatty acid (TFA) content which comprises the ω6 fatty acids, wherein at least some of the ω6 fatty acids are esterified in the form of phospholipids in the polar lipid, and wherein the ω6 fatty acids comprise two, three, four or more fatty acids selected from the group consisting of arachidonic acid (ARA), dihomo-γ-linolenic acid (DGLA), eicosadienoic acid (EDA), docosatetraenoic acid (DTA), docosapentaenoic acid-ω6 (DPA-ω6) and γ-linolenic acid (GLA),
(b) the polar lipid comprises a total saturated fatty acid content comprising palmitic acid and stearic acid,
(c) the polar lipid comprises a total monounsaturated fatty acid content comprising oleic acid and palmitoleic acid (C16:1Δ9cis), and
(d) the polar lipid lacks C16:2, C16:3ω3, EPA and DHA.

In another aspect, the present invention provides an extracted microbial lipid, comprising esterified fatty acids in the form of either (i) polar lipid without any non-polar lipid, or (ii) polar lipid and non-polar lipid, the polar lipid preferably being present in the extracted microbial lipid in a greater amount than the non-polar lipid, wherein
(a) the polar lipid of (i) and (ii) comprises a total fatty acid (TFA) content which comprises the ω6 fatty acids, wherein at least some of the ω6 fatty acids are esterified in the form of phospholipids in the polar lipid, and wherein the ω6 fatty acids of the polar lipid comprise an amount of arachidonic acid (ARA), dihomo-γ-linolenic acid (DGLA), eicosadienoic acid (EDA), docosatetraenoic acid (DTA), docosapentaenoic acid-ω6 (DPA-ω6) or γ-linolenic acid (GLA), or any combination thereof, each amount being expressed as a weight percentage of the total fatty acid content of the polar lipid, whereby the sum of the amounts of ARA, DGLA, EDA, DTA, DPA-ω6 and GLA is at least about 10%,
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis).

In another aspect, provided is an extracted yeast lipid, comprising esterified fatty acids in the form of either (i) polar lipid without any non-polar lipid, or (ii) polar lipid and non-polar lipid, wherein
(a) the polar lipid of (i) and (ii) comprises a total fatty acid (TFA) content which comprises the ω6 fatty acids, wherein at least some of the ω6 fatty acids are esterified in the form of phospholipids in the polar lipid, and wherein the ω6 fatty acids of the polar lipid comprise an amount of arachidonic acid (ARA), dihomo-γ-linolenic acid (DGLA), eicosadienoic acid (EDA), docosatetraenoic acid (DTA), docosapentaenoic acid-ω6 (DPA-ω6) or γ-linolenic acid (GLA), or any combination thereof, whereby the sum of the amounts of ARA, DGLA, EDA, DTA, DPA-ω6 and GLA is preferably at least about 5%, more preferably at least about 10%, by weight of the TFA content of the polar lipid,
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis).

In another aspect, provided is an extracted Saccharomyces cerevisiae lipid, comprising ω6 fatty acids esterified in the form of polar lipid, wherein
(a) the polar lipid comprises a total fatty acid (TFA) content which comprises the ω6 fatty acids, wherein at least some of the ω6 fatty acids are esterified in the form of phospholipids in the polar lipid, and wherein the ω6 fatty acids one, two, three, four or more fatty acids selected from the group consisting of arachidonic acid (ARA), dihomo-γ-linolenic acid (DGLA), eicosadienoic acid (EDA), docosatetraenoic acid (DTA), docosapentaenoic acid-ω6 (DPA-ω6) and γ-linolenic acid (GLA),
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis).

Also provided is an extracted microbial lipid comprising esterified fatty acids in the form of either (i) polar lipid without any non-polar lipid, or (ii) polar lipid and non-polar lipid, the polar lipid being present in the extracted microbial lipid in a greater amount than the non-polar lipid, wherein
(a) the polar lipid of (i) and (ii) comprises a total fatty acid (TFA) content which comprises ω6 fatty acids, wherein at least some of the ω6 fatty acids are esterified in the form of phospholipids in the polar lipid, the ω6 fatty acids comprising arachidonic acid (ARA), dihomo-γ-linolenic acid (DGLA), and γ-linolenic acid (GLA), wherein ARA is present in an amount of about 10% to about 60% of the total fatty acid content of the polar lipid, DGLA is present in an amount of about 0.1% to about 5% of the total fatty acid content of the polar lipid and GLA is present in an amount of about 1% to about 10% of the total fatty acid content of the polar lipid,
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis), wherein when the composition is heated, one or more compounds which have a meat-associated flavour and/or aroma are produced.

In the above aspect, ARA may present in an amount of about 20% to about 50% of the total fatty acid content of the polar lipid, DGLA may be present in an amount of about 1% to about 5% of the total fatty acid content of the polar lipid and GLA may be present in an amount of about 3% to about 10%. In particular examples, ARA is present in an amount of about 25% to about 50%, or about 30% to about 50%. In other examples, ARA is present in an amount of about 10% to about 25% (or 10% to 20%) of the total fatty acid content of the polar lipid, DGLA is present in an amount of about 0.5% to about 5% of the total fatty acid content of the polar lipid and GLA is present in an amount of about 3% to about 10%.

Also provided is an extracted microbial lipid comprising esterified fatty acids in the form of either (i) polar lipid without any non-polar lipid, or (ii) polar lipid and non-polar lipid, the polar lipid being present in the extracted microbial lipid in a greater amount than the non-polar lipid, wherein
(a) the polar lipid comprises a total fatty acid (TFA) content which comprises ω6 fatty acids, wherein the ω6 fatty acids are present in an amount of about 30% to about 70% of the total fatty acid content of the polar lipid and wherein at least some of the ω6 fatty acids are esterified in the form of phospholipids in the polar lipid, the ω6 fatty acids comprising arachidonic acid (ARA), dihomo-γ-linolenic acid (DGLA), and γ-linolenic acid (GLA),
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis) wherein when the composition is heated, one or more compounds which have a meat-associated flavour and/or aroma are produced.

In some examples, the ω6 fatty acids are present in an amount of about 40% to about 70%, about 40% to about 60%, or about 50% to about 60% of the total fatty acid content of the polar lipid. In one example, ARA is present in an amount of about 20% to about 50% (e.g. 25% to about 50%, or about 30% to about 50%) of the total fatty acid content of the polar lipid, DGLA is present in an amount of about 1% to about 5% of the total fatty acid content of the polar lipid and GLA is present in an amount of about 3% to about 10%.

The ratio of polar lipid to non-polar lipid in the extracted microbial lipid of the present invention may be at least 1.1:1, at least 1.5:1, at least 2:1, at least 3:1, at least 4:1, at least 5:1, at least 6:1, at least 7:1, at least 8:1, at least 9:1, at least 10:1, between 1.1:1 and 10:1, between 1.1:1 and 5:1 or between 1.1:1 and 25.1:1.

In one embodiment, if the polar lipid comprises DPA-ω6, one or more or all of GLA, DGLA, EDA, ARA and DTA are also present. In an embodiment, if the polar lipid comprises DPA-ω6, one or more or all of ARA, EPA and DHA are also present.

In one embodiment, the polar lipid comprises EDA and one, two or all three of arachidonic acid (ARA), dihomo-γ-linolenic acid (DGLA) and γ-linolenic acid (GLA) esterified in the polar lipid, and wherein the level of EDA in the polar lipid is at least about 1% of the total fatty acid content of the polar lipid.

In one embodiment, the polar lipid lacks one, two, three or all four of C16:2, C16:3ω3, EPA and DHA. In a preferred embodiment, the polar lipid lacks C16:3ω3, EPA and DHA. In a further embodiment, the polar lipid also lacks α-linolenic acid (ALA) or has less than 2% or less than 1% ALA. In a further embodiment, the polar lipid also lacks EPA or has less than 2% or less than 1% EPA. In a further embodiment, the polar lipid also lacks DHA or has less than 2% or less than 1% DHA.

In an embodiment, ω3 fatty acids are present in a total amount of less than about 2%, less than about 1%, or between 3% and 0.1%, by weight of the TFA content of the polar lipid.

In one embodiment, the extracted lipid comprises three, four or more fatty acids selected from the group consisting of ARA, DGLA, EDA, DTA, DPA-ω6 and GLA, such as a combination of ARA, DGLA and GLA, or a combination of fatty acids other than ARA, DGLA and GLA, preferably a combination of ARA, DGLA, GLA and at least one of EDA, DTA and DPA-ω6. In an embodiment, the sum total of the amounts of ARA, DGLA, EDA, DTA, DPA-ω6 and GLA is between about 10% and about 70%, or between about 10% and about 75% or between about 10% and about 80%, each amount being expressed as a percentage of the total fatty acid content of the polar lipid. In an embodiment, the ω6 fatty acid that is present in the greatest amount in the total fatty acid content of the polar lipid is not LA, or not ARA. In an embodiment, if the ω6 fatty acid that is present in the greatest amount is GLA or DGLA, the polar lipid comprises one or more of EDA, DTA or DPA-ω6.

In one embodiment, the phospholipids in the polar lipid comprise at least two, at least three or all four of phosphatidylcholine (PC), phosphatidylethanolamine (PE), phosphatidylinositol (PI) and phosphatidylserine (PS), each comprising one, two, three or more than three of ARA, DGLA, EDA, DTA, DPA-ω6 and GLA, and optionally one or more or all of phosphatidic acid (PA), phosphatidylglycerol (PG) and cardiolipin (Car), each comprising one, two, three or more than three of ARA, DGLA, EDA, DTA, DPA-ω6 and GLA.

In one embodiment, the polar lipid comprises myristic acid (C14:0) in an amount of less than about 2% by weight of the total fatty acid content of the polar lipid. In a preferred embodiment, the polar lipid comprises myristic acid (C14:0) in an amount of less than about 1% by weight of the total fatty acid content of the polar lipid.

In embodiments, stearic acid is present at a level of less than about 14% or less than about 12% or less than about 10% of the total fatty acid content of the polar lipid. In preferred embodiments, stearic acid is present at a level of less than about 7% or less than about 6% or less than about 5%, preferably less than 4% or less than 3%, of the total fatty acid content of the polar lipid.

In embodiments, ARA is present in an amount of about 10% to about 60%, about 10% to about 30%, about 10% to about 25%, about 15% to about 60%, about 20% to about 60%, or about 30% to about 60%, by weight of the TFA content of the polar lipid. In preferred embodiments, ARA is present in an amount of about 20% to about 60%, or about 30% to about 60%, or about 40% to about 60%, or about 50% to about 60%, by weight of the TFA content of the polar lipid.

In one embodiment, the extracted microbial lipid is extracted eukaryotic microbial lipid. In one embodiment, the extracted microbial lipid is extracted fungal microbial lipid.

In one embodiment, the extracted microbial lipid is extracted fungal lipid, for example from a filamentous fungus or mold, or a eukaryotic microbial lipid. In an embodiment, the extracted fungal lipid is Mortierella sp., such as Mortierella alpina or Mortierella elongata, lipid. In an embodiment, the extracted fungal lipid is from the Genus Mucor, for example from the species Mucor hiemalis.

In one embodiment, the extracted microbial lipid is an extracted yeast lipid, preferably a Saccharomyces cerevisiae, Yarrowia lipolytica, or Pichia pastoris lipid.

In one embodiment, the polar lipid comprises one or more or all of EDA, DTA and DPA-ω6.

In one embodiment, if the polar lipid comprises DGLA and ARA, or GLA, DGLA and ARA, then at least one of the following apply:
(a) at least one of EDA, DTA and DPA-ω3 is also present in the polar lipid; and
(b) the ratio of PC to PE or to phospholipids other than PC is less than 3:1, less than 2:1, less than 1.5:1, less than 1.25:1, less than 1:1, between 3:1 and 1:1, between 2:1 and 1:1, or between 3:1 and 0.5:1.

In one embodiment, GLA is present in the polar lipid in an amount which is (i) less than the sum of the amounts of ARA, DGLA, EDA, DTA and DPA-ω6 in the polar lipid, or (ii) one or more of: less than the amount of ARA, less than the amount of DGLA, less than the amount of EDA, less than the amount of DTA and less than the amount of DPA-ω6, or any combination thereof, in the polar lipid.

In embodiments, the saturated fatty acid content of the polar lipid comprises one or more or all of lauric acid (C12:0), myristic acid (C14:0), a C15:0 fatty acid, C20:0, C22:0 and C24:0, preferably comprising C14:0 and C24:0 or C14:0, C15:0 and C24:0, more preferably comprising C14:0, C15:0 and C24:0 but not C20:0 and C22:0.

In embodiments, lauric acid and myristic acid are absent from the polar lipid, or lauric acid and/or myristic acid is present in the polar lipid, whereby the sum of the amounts of lauric acid and myristic acid in the polar lipid is less than about 2%, or less than about 1%, preferably less than about 0.5%, more preferably less than about 0.2%, of the total fatty acid content of the polar lipid.

In embodiments, C15:0 is absent from the polar lipid, or C15:0 is present in the polar lipid in an amount of less than about 3%, preferably less than about 2% or less than about 1%, of the total fatty acid content of the polar lipid.

In embodiments, palmitic acid is present in the polar lipid in an amount of about 3% to about 45%, or about 10% to about 40%, or about 20% to about 45%, of the total fatty acid content of the polar lipid.

In embodiments, palmitoleic acid is present in the polar lipid in an amount of about 3% to about 45%, or about 3% to about 25%, or about 3% to about 20%, or about 3% to about 15%, of the total fatty acid content of the polar lipid.

In embodiments, oleic acid is present in the polar lipid in an amount of about 3% to about 60%, or about 3% to about 40%, or about 3% to about 25%, or about 20% to about 60%, of the total fatty acid content of the polar lipid.

In embodiments, vaccenic acid is absent from the polar lipid, or vaccenic acid is present in the polar lipid in an amount of less than about 2%, preferably less than about 1% or about 0.5%, of the total fatty acid content of the polar lipid.

In embodiments, linoleic acid is present in the polar lipid in an amount of about 3% to about 45%, or about 3% to about 30%, or about 3% to about 20%, of the total fatty acid content of the polar lipid.

In embodiments, y-linoleic acid is absent from the polar lipid, or y-linoleic acid is present in the polar lipid in an amount of about 3% to about 12%, or about 3% to about 8%, or about 3% to about 6%, or less than about 3% of the total fatty acid content of the polar lipid.

In embodiments, eicosadienoic acid is absent from the polar lipid, or eicosadienoic acid is present in the polar lipid in an amount of about 3% to about 12%, or about 3% to about 8%, or about 3% to about 6%, or less than about 3% of the total fatty acid content of the polar lipid.

In embodiments, dihomo-γ-linolenic acid is absent from the polar lipid, or dihomo-γ-linolenic acid is present in the polar lipid, preferably in an amount of less than about 2%, 0.1% to about 2%, about 10% to about 60%, about 12% to about 60% or about 15% to about 60%, of the total fatty acid content of the polar lipid.

In embodiments, C20:0 and C22:0 are absent from the polar lipid, or C20:0 and/or C22:0 is present in the polar lipid, whereby the sum of the amounts of C20:0 and C22:0 in the polar lipid is less than about 1.0% or less than about 0.5%, preferably less than 0.2%, of the total fatty acid content of the polar lipid.

In embodiments, C24:0 is absent from the polar lipid, or C24:0 is present in the polar lipid in an amount of less than about 1.0%, less than about 0.5%, preferably less than 0.3% or less than 0.2%, of the total fatty acid content of the polar lipid.

In embodiments, C17:1 is absent from the polar lipid, or C17:1 is present in the polar lipid in an amount of less than about 5%, preferably less than about 4% or less than about 3%, more preferably less than about 2% of the total fatty acid content of the polar lipid.

In embodiments, monounsaturated fatty acids which are C20 or C22 fatty acids are absent from the polar lipid, or C20:1 and/or C22:1 is present in the polar lipid, whereby the sum of the amounts of C20:1 and C22:1 in the polar lipid is less than about 1.0%, less than about 0.5%, preferably less than 0.2%, of the total fatty acid content of the polar lipid.

In embodiments, the content of ω6 fatty acids in the polar lipid which are (i) C20 or C22 fatty acids is about 5% to about 60%, preferably about 10% to about 60% of the total fatty acid content of the polar lipid, and/or (ii) ω6 fatty acids which have 3, 4 or 5 carbon-carbon double bonds, is about 5% to about 70%, preferably about 10% to about 70%, more preferably about 40% to about 70% or about 45% to about 70% or about 50% to about 70% of the total fatty acid content of the polar lipid.

In embodiments, C16:3ω3 is absent from the polar lipid, or both C16:2 and C16:3ω3 are absent from the polar lipid.

In embodiments, the extracted lipid comprises PC and/or lacks cyclopropane fatty acids, preferably lacks C15:0c, C17:0c and C19:0c.

In an embodiment, the weight of the extracted microbial lipid is at least 100 mg, preferably at least 1 g. In an embodiment, the extracted microbial lipid is in a liquid form with a volume of at least 1 ml, preferably at least 10 ml.

### Fatty Acid Biosynthesis

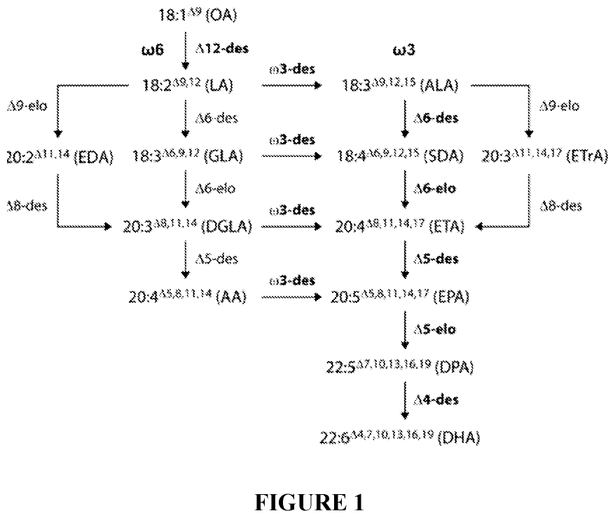

Biosynthesis of ω6 fatty acids in organisms such as microalgae, mosses and fungi usually occurs as a series of oxygen-dependent desaturation and elongation reactions (FIG. 1). Polynucleotides encoding these enzymes can be used to genetically engineer microbes to produce the extracted lipid of the present invention. The desaturase and elongase proteins, and genes encoding them, that may be used in the invention are any of those known in the art or homologues or derivatives thereof. The desaturase enzymes that have been shown to participate in ω6 fatty acid biosynthesis all belong to the group of so-called “front-end” desaturases. Preferred proteins, or combinations of proteins, are those encoded by the genetic constructs provided herein, for example the amino acid sequences provided as SEQ ID NOs: 1 to 19.

Activity of any of the elongases or desaturases for use in the invention may be tested by expressing a gene encoding the enzyme in a microbial cell such as, for example, a yeast cell, and determining whether the cell has an increased capacity to produce ω6 fatty acids compared to a comparable cell in which the enzyme is not expressed.

Whilst certain enzymes are specifically described herein as “bifunctional”, the absence of such a term does not necessarily imply that a particular enzyme does not possess an activity other than that specifically defined.

### Desaturases

As used herein, the term “desaturase” refers to an enzyme which is capable of introducing a carbon-carbon double bond into the acyl group of a fatty acid substrate which is typically in an esterified form such as, for example, acyl-CoA esters. The acyl group may be esterified to a phospholipid such as phosphatidylcholine (PC), or to acyl carrier protein (ACP), or preferably to CoA. Desaturases generally may be categorized into three groups accordingly.

In one embodiment, the desaturase is a front-end desaturase.

As used herein, the term “front-end desaturase” refers to a member of a class of enzymes that introduce a double bond between the carboxyl group and a pre-existing unsaturated part of the acyl chain of lipids, which are characterized structurally by the presence of an N-terminal cytochrome b5 domain, along with a typical fatty acid desaturase domain that includes three highly conserved histidine boxes (Napier et al., 1997).

As used herein, a “Δ5-desaturase” refers to a protein which is capable of performing a desaturase reaction that introduces a carbon-carbon double bond at the 5^{th} carbon-carbon bond from the carboxyl end of a fatty acid substrate. In an embodiment, the fatty acid substrate is DGLA and the enzyme produces ARA. In an embodiment, the Δ5-desaturase has greater activity on an ω6 fatty acid when compared to a corresponding ω3 fatty acid. In one embodiment, the “Δ5-desaturase” is capable of converting DGLA-CoA to ARA-CoA, i.e. it is an acyl-CoA desaturase. In an embodiment, the “Δ5-desaturase” is capable of converting DGLA esterified at the sn-2 position of PC. Examples of Δ5-desaturases are listed in Ruiz-Lopez et al. (2012) and Petrie et al. (2010a). In one embodiment, the Δ5-desaturase comprises amino acids having a sequence as provided in SEQ ID NO:15, a biologically active fragment thereof, or an amino acid sequence which is at least 60% identical to SEQ ID NO:15. In another embodiment, the Δ5-desaturase comprises amino acids having a sequence as provided in SEQ ID NO: 16, a biologically active fragment thereof, or an amino acid sequence which is at least 60% identical to SEQ ID NO:16. In another embodiment, the Δ5-desaturase is from Pavlova salina or Mortierella alpina.

As used herein, a “A6-desaturase” refers to a protein which is capable of performing a desaturase reaction that introduces a carbon-carbon double bond at the 6^{th} carbon-carbon bond from the carboxyl end of a fatty acid substrate. Preferably, the Δ6-desaturase has greater activity on an ω6 fatty acid when compared to a corresponding ω3 fatty acid. In an embodiment, the fatty acid substrate is LA and the enzyme produces GLA. In one embodiment, the “A6-desaturase” is capable of converting LA-CoA to GLA-CoA, i.e. it is an acyl-CoA desaturase. In an embodiment, the “Δ6-desaturase” is capable of converting LA esterified at the sn-2 position of PC. In a further embodiment, the Δ6-desaturase has activity on both fatty acid substrates LA-CoA and on LA joined to the sn-2 position of PC. Preferably the Δ6-desaturase has greater activity on LA-CoA than on LA-PC. The Δ6-desaturase may also have activity as a Δ5-desaturase, in which case it is termed a Δ5/Δ6 bifunctional desaturase, so long as it has greater Δ6-desaturase activity on LA than Δ5-desaturase activity on DGLA. Examples of 46-desaturases are listed in Ruiz-Lopez et al. (2012) and Petrie et al. (2010a). Preferred A6-desaturases are from Mortierella alpina or Ostreococcus tauri.

In an embodiment, the Δ6-desaturase is further characterised by having greater 46-desaturase activity on linoleic acid (LA, C18:2Δ9,12, ω6) than α-linolenic acid (ALA, C18:3Δ9,12,15, ω3) as fatty acid substrate.

In one embodiment, the Δ6-desaturase has no detectable Δ5-desaturase activity on ETA.

In another embodiment, the Δ6-desaturase comprises amino acids having a sequence as provided in SEQ ID NO:5 or SEQ ID NO:6 or, a biologically active fragment thereof, or an amino acid sequence which is at least 60% identical to SEQ ID NO:5 or SEQ ID NO:6. The Δ6-desaturase may also have Δ8-desaturase activity, or not.

As used herein, a “Δ8-desaturase” refers to a protein which is capable of performing a desaturase reaction that introduces a carbon-carbon double bond at the 8^{th} carbon-carbon bond from the carboxyl end of a fatty acid substrate. The Δ8-desaturase is at least capable of converting EDA to DGLA. In an embodiment, the Δ8-desaturase is capable of converting EDA-CoA to DGLA-CoA, i.e. it is an acyl-CoA desaturase. In an embodiment, the Δ8-desaturase is capable of converting EDA esterified at the sn-2 position of PC. Preferably the Δ8-desaturase has greater activity on EDA-CoA than on EDA-PC. The Δ8-desaturase may also have activity as a Δ6-desaturase, being termed a Δ6/Δ8 bifunctional desaturase, so long as it has greater Δ8-desaturase activity on EDA than Δ6-desaturase activity on LA. In one embodiment, the Δ8-desaturase comprises amino acids having a sequence as provided in SEQ ID NO: 14, a biologically active fragment thereof, or an amino acid sequence which is at least 60% identical to SEQ ID NO:14. In one embodiment, the Δ8-desaturase is a Pavlova salina Δ8-desaturase.

As used herein, a “Δ12-desaturase” refers to a protein which is capable of performing a desaturase reaction that introduces a carbon-carbon double bond at the 12^{th} carbon-carbon bond from the carboxyl end of a fatty acid substrate. Δ12-desaturases typically convert either oleoyl-phosphatidylcholine or oleoyl-CoA to linoleoyl-phosphatidylcholine (C18:1-PC) or linoleoyl-CoA (C18:1-CoA), respectively. The subclass using the PC linked substrate are referred to as phospholipid-dependent Δ12-desaturases, the latter subclass as acyl-CoA dependent Δ12-desaturases. Plant and fungal Δ12-desaturases are generally of the former sub-class, whereas animal Δ12-desaturases, with the exception of some lower animal Δ12-desaturases such as C. elegans Δ12-desaturase, are generally of the latter subclass, for example the Δ12-desaturases encoded by genes cloned from insects by Zhou et al. (2008). Many other Δ12-desaturase sequences can be easily identified by searching sequence databases. In one embodiment, the Δ12-desaturase comprises amino acids having a sequence as provided in any one of SEQ ID

NOs: 1 to 4, a biologically active fragment thereof, or an amino acid sequence which is at least 60% identical to SEQ ID NOs: 1 to 4. In one embodiment, the Δ12-desaturase is a Lachancea kluyveri, Y. lipolytica, Acheta domesticus or Fusarium moniliforme Δ12-desaturase. In a preferred embodiment, the Δ12-desaturase is a fungal Δ12-desaturase or fungal. As used herein, a “fungal Δ12-desaturase” refers to a Δ12-desaturase which is from a fungal source, including an oomycete source, or a variant thereof whose amino acid sequence is at least 95% identical thereto. Genes encoding numerous desaturases have been isolated from fungal sources. U.S. Pat. No. 7,211,656 describes a Δ12 desaturase from Saprolegnia diclina. WO2009016202 describes fungal desaturases from Helobdella robusta, Laccaria bicolor, Lottia gigantea, Microcoleus chthonoplastes, Monosiga brevicollis, Mycosphaerella fijiensis, Mycospaerella graminicola, Naegleria gruben, Nectria haematococca, Nematostella vectensis, Phycomyces blakesleeanus, Trichoderma resii, Physcomitrella patens, Postia placenta, Selaginella moellendorffii and Microdochium nivale. WO2005/012316 describes a Δ12-desaturase from Thalassiosira pseudonana and other fungi. WO2003/099216 describes genes encoding fungal Δ12-desaturases isolated from Neurospora crassa, Aspergillus nidulans, Botrytis cinerea and Mortierella alpina.

As used herein, a “Δ4-desaturase” refers to a protein which is capable of performing a desaturase reaction that introduces a carbon-carbon double bond at the 4^{th} carbon-carbon bond from the carboxyl end of a fatty acid substrate. The Δ4-desaturase is at least capable of converting DTA to DPA-ω6 (C22:5Δ4,7,10,13,16). Preferably, the Δ4-desaturase has greater activity on an ω6 fatty acid when compared to a corresponding ω3 fatty acid. In one embodiment, the Δ4-desaturase is capable of converting DTA-CoA to DPAω6-CoA, i.e. it is an acyl-CoA desaturase. In an embodiment, the Δ4-desaturase is capable of converting DTA esterified at the sn-2 position of PC to DPAω6-PC. The desaturation step to produce DPAω6 from DTA is catalysed by a Δ4-desaturase in organisms other than mammals, and a gene encoding this enzyme has been isolated from the freshwater protist species Euglena gracilis and the marine species Thraustochytrium sp. (Qiu et al., 2001; Meyer et al., 2003). In one embodiment, the Δ4-desaturase comprises amino acids having a sequence as provided in SEQ ID NO: 18, or a Pavlova spp. Δ4-desaturase such as a Pavlova salina Δ4-desaturase, a biologically active fragment thereof, or an amino acid sequence which is at least 60% identical to SEQ ID NO:18. In one embodiment, the Δ4-desaturase comprises amino acids having a sequence as provided in SEQ ID NO:19, or a Thraustochytrium sp. Δ4-desaturase, a biologically active fragment thereof, or an amino acid sequence which is at least 60% identical to SEQ ID NO: 19.

In an embodiment, a desaturase for use in the present invention has greater activity on an acyl-CoA substrate than a corresponding acyl-PC substrate. In another embodiment, a desaturase for use in the present invention has greater activity on an acyl-PC substrate than a corresponding acyl-CoA substrate, but has some activity on both substrates. As outlined above, a “corresponding acyl-PC substrate” refers to the fatty acid esterified at the sn-2 position of phosphatidylcholine (PC) where the fatty acid is the same fatty acid as in the acyl-CoA substrate. In an embodiment, the greater activity is at least two-fold greater. To test which substrate a desaturase acts on, namely an acyl-CoA or an acyl-PC substrate, assays can be carried out in yeast cells as described in Domergue et al. (2003 and 2005). Acyl-CoA substrate capability for a desaturase can also be inferred when an elongase, when expressed together with the desaturase, has a high enzymatic conversion efficiency, such as for example of at least about 90% where the elongase catalyses the elongation of the product of the desaturase.

### Elongases

Biochemical evidence suggests that the fatty acid elongation consists of 4 steps: condensation, reduction, dehydration and a second reduction. In the context of this invention, an “elongase” refers to the polypeptide that catalyses the condensing step in the presence of the other members of the elongation complex, under suitable physiological conditions. It has been shown that heterologous or homologous expression in a cell of only the condensing component (“elongase”) of the elongation protein complex is required for the elongation of the respective acyl chain. Thus, the introduced elongase is able to successfully recruit the reduction and dehydration activities from the transgenic host to carry out successful acyl elongations. The specificity of the elongation reaction with respect to chain length and the degree of desaturation of fatty acid substrates is thought to reside in the condensing component. This component is also thought to be rate limiting in the elongation reaction.

As used herein, a “Δ6-elongase” is at least capable of converting GLA to DGLA. In one embodiment, the elongase comprises amino acids having a sequence as provided in SEQ ID NO: 13, a biologically active fragment thereof, or an amino acid sequence which is at least 60% identical to SEQ ID NO:13. In an embodiment, the Δ6-elongase is from Physcomitrella patens (Zank et al., 2002; Accession No. AF428243) or Thalassiosira pseudonana (Ruiz-Lopez et al., 2012). In a preferred embodiment, the Δ6-elongase is from Pyramimonas cordata. In a further embodiment, the Δ6-elongase has greater activity on an ω6 substrate than the corresponding ω3 substrate.

As used herein, a “Δ9-elongase” is at least capable of converting LA to EDA. In one embodiment, the Δ9-elongase comprises amino acids having a sequence as provided in any one of SEQ ID NOs: 9 to 12, a biologically active fragment thereof, or an amino acid sequence which is at least 60% identical to any one of SEQ ID NOs: 9 to 12. In a further embodiment, the Δ9-elongase has greater activity on an ω6 substrate than the corresponding ω3 substrate.

As used herein, the term “has greater activity on an ω6 substrate than the corresponding ω3 substrate” refers to the relative activity of the enzyme on substrates that differ by the action of an ω3 desaturase.

An elongase for use in the present invention has activity only on an acyl-CoA substrate, not on a corresponding acyl-PC substrate.

### Other Genes

In addition to expression of the above desaturases and elongases, production of ω6 fatty acids in the polar lipid of microbial cells can be enhanced by genetic modification to modulate expression of one or more endogenous genes involved in microbial fatty acid biosynthesis, catabolism and regulation. Such exemplary microbial genes are provided in Table 1.

In some embodiments, the genetic modification(s) that increase the production of ω6 fatty acids in the polar lipid provide for increased expression and/or activity of one or more genes in Table 1. In some embodiments, the genetic modification(s) provide for increased expression and/or activity of a fatty acid synthesis gene (see Table 1 for examples). In some embodiments, the genetic modification(s) provide for increased expression and/or activity of a phospholipid synthesis gene (see Table 1 for examples). In some embodiments, the genetic modification(s) provide for increased expression and/or activity of a lipid synthesis regulating gene (see Table 1 for examples).

In some embodiments, the genetic modification(s) that increase the production of ω6 fatty acids in the polar lipid reduce or prevent expression and/or activity of one or more genes in Table 1. In some embodiments, the genetic modification(s) reduce or prevent expression and/or activity of a lipid catabolism gene (see Table 1 for examples).

TABLE 1
Microbial genes and the accession numbers of encoded proteins involved
in fatty acid and lipid synthesis, catabolismand regulation.

| | | | | | Gene in |
|---|---|---|---|---|---|
| | Enzyme | | Gene in | Gene in | Pichia |
| Enzyme or protein | Commission No. | Gene | S. cerevisiae | Y. lipolytica | pastoris |
| Fatty acid synthesis | | | | | |
| and TAG production | | | | | |
| Acetyl-CoA | EC 6.2.1.1 | ACS1 | YAL054C | YALI0F05962g | ANZ76230.1 |
| synthetase | | | | | |
| Acetyl-CoA | EC 6.2.1.1 | ACS2 | YLR153C | none | ANZ73211.1 |
| synthetase | | | | | |
| ATP-citrate lyase | EC 2.3.3.8 | ACL1 | none | YALI0E34793g | ANZ75267.1 |
| subunit | | | | | |
| ATP-citrate lyase | EC 2.3.3.8 | ACL2 | none | YALI0D24431g | none |
| subunit | | | | | |
| Acetyl-CoA | EC 6.3.4.14 & | ACC1 | FAS3 | YALI0C11407g | ANZ73439.1 |
| carboxylase | EC 6.4.1.2 | | (accession | | |
| | | | no. | | |
| | | | M92156) | | |
| | | | YNR016C | | |
| Fatty acid synthase | EC 2.3.1.86 | FAS1 | YKL182W | YALI0B15059g | ANZ74209.1 |
| subunit beta | | | | | |
| Fatty acid synthase | EC 2.3.1.86 | FAS2 | YPL231W | YALI0B19382g | ANZ73614.1 |
| subunit alpha | | | | | |
| Fatty acid elongase | EC 2.3.1.199 | ELO1 | YJL196C | YALI0F06754g | ANZ76899.1 |
| Fatty acid elongase | EC 2.3.1.199 | ELO2 | YCR034W | YALI0B20196g | ANZ74046.1 |
| Fatty acid elongase | EC 2.3.1.199 | ELO3 | YLR372W | none | ANZ73325.1 |
| Δ5 Fatty acid | EC 1.14.19.30 | DES1 | none | none | none |
| desaturase | | | | | |
| Δ9 Fatty acid | EC 1.14.19.1 | OLE1 or | YGL055W | YALI0C05951g | ANZ77426.1 |
| desaturase | | SCD | | | |
| Δ12 Fatty acid | EC 1.14.19.6 | FAD2 | none | YALI0B10153g | AAX20125.1 |
| desaturase | | | | | |
| Δ15/ω3 Fatty | EC: 1.14.19.13 | FAD3 | none | none | ABL63813.1 |
| acid | | | | | |
| desaturase | | | | | |
| Glycerol kinase | EC 2.7.1.30 | GUT1 | YHL032C | YALI0F00484g | ANZ77337.1 |
| G3P dehydrogenase | EC 1.1.1.18 | GPD1 | YDL022W | YALI0B02948g | ANZ75813.1 |
| (NAD) | | | | | |
| G3P dehydrogenase | EC 1.1.1.18 | GPD2 | YOL059W | none | none |
| (NAD) | | | | | |
| G3P dehydrogenase | EC 1.1.99.5 | GUT2 | YIL155C | YALI0B13970g | ANZ73466.1 |
| (mitochondria) | | | | | |
| Glycerophosphate | EC 2.3.1.15 | SCT1 | YBL011W | YALI0C00209g | ANZ74560.1 |
| acyltransferase | | | | | |
| (GPAT) | | | | | |
| Glycerophosphate | EC 2.3.1.15 & | GPT2 | YKR067W | none | ANZ73642.1 |
| acyltransferase | EC 2.3.1.42 | | | | |
| (GPAT) | | | | | |
| 1-Acyl-sn- | EC 2.3.1.51 | SLC1 | YDL052C | YALI0E18964g | ANZ73792.1 |
| glycerophosphate | | | | | |
| acyltransferase | | | | | |
| (LPAAT) | | | | | |
| 1-Acyl-sn- | EC 2.3.1.51 | ALE1 | YOR175C | YALI0F19514g | ANZ74296.1 |
| glycerophosphate | | | | | |
| acyltransferase | | | | | |
| (LPAAT) | | | | | |
| 1-Acyl-sn- | EC 2.3.1.51 | LOA | YPR139C | YALI0C14014g | ANZ76093.1 |
| glycerophosphate | | | | | |
| acyltransferase | | | | | |
| (LPAAT) | | | | | |
| Phosphatidic acid | EC 3.1.3.4 | PAH1 | YMR165C | YALI0D27016g | ANZ74484.1 |
| phosphatase (PAP) - | | | | | |
| phosphohydrolase | | | | | |
| Phosphatidic acid | EC 3.1.3.4 | APP1 | YNL094W | YALI0D02233g | ANZ75274.1 |
| phosphatase (PAP) | | | | | |
| Diacylglycerol | EC 2.3.1.20 | DGA1 | YOR245C | YALI0E32769g | ANZ74314.1 |
| acyltransferase | | | | | |
| (DGAT) | | | | | |
| Diacylglycerol | EC 2.3.1.20 | DGA2 | none | YALI0D07986g | none |
| acyltransferase | | | | | |
| (DGAT) | | | | | |
| Phospholipid:diacyl- | EC 2.3.1.158 | LRO1 | YNR008W | YALI0E16797g | ANZ75160.1 |
| glycerol | | | | | |
| acyltransferase | | | | | |
| (PDAT) | | | | | |
| Acyl-CoA:sterol | EC 2.3.1.26 | ARE1 | YCR048W | YALI0F06578g | ANZ73460.1 |
| acyltransferase | | | | | |
| Acyl-CoA:sterol | EC 2.3.1.26 | ARE2 | YNR019W | none | none |
| acyltransferase | | | | | |
| Cardiolipin synthase | EC 2.7.8.5 | PGS1 | YCL004W | YALI0F23837g | ANZ73566.1 |
| Fatty acyl-CoA | EC 6.2.1.3 | FAA1 | YOR317W | YALI0D17864g | ANZ75849.1 |
| synthetase | | | | | |
| Enzymes involved in | | | | | |
| PL synthesis | | | | | |
| Inositol-3-phosphate | EC 5.5.1.4 | INO1 | YJL153C | YALI0B04312g | AAC33791.1 |
| synthase | | | | | |
| Phosphatidate | EC 2.7.7.41 | CDS1 | YBR029C | YALI0E14443g | ANZ74906.1 |
| cytidylyltransferase | | | | | |
| CDP-DAG synthase | EC 2.7.7.41 | TAM41 | YGR046W | YALI0C12276g | ANZ73236.1 |
| Phosphatidylinositol | EC 2.7.8.11 | PIS1 | YPR113W | YALI0F20328g | ANZ76102.1 |
| synthase | | | | | |
| Phosphatidylserine | EC 2.7.8.8 | CHO1 | YER026C | YALI0D08514g | ANZ74203.1 |
| synthase | | | | | |
| Phosphatidylserine | EC 4.1.1.65 | PSD1 | YNL169C | YALI0D21604g | ANZ73933.1 |
| decarboxylase | | | | | |
| Phosphatidylserine | EC 4.1.1.65 | PSD2 | YGR170W | YALI0D03480g | ANZ76834.1 |
| decarboxylase | | | | | |
| Phosphatidyl- | EC 2.1.1.17 | CHO2 | YGR157W | YALI0E06061g | ANZ73427.1 |
| ethanolamine | | | | | |
| methyltransferase | | | | | |
| Phospholipid | EC 2.1.1.17 & | OPI3 | YJR073C | YALI0E12441g | ANZ76546.1 |
| methyltransferase | EC 2.1.1.71 | | | | |
| Phosphatidylinositol | | PDR16 | YNL231C | YALI0A08448g | ANZ78024.1 |
| transfer protein | | | | | |
| Phosphatidylinositol/ | | CSR1 | YLR380W | YALI0C17545g | ANZ76329.1 |
| phosphatidylcholine | | | | | |
| transfer protein | | | | | |
| Phosphatidylinositol | EC 3.1.3.36 | SAC1 | YKL212W | YALI0D05995g | ANZ76554.1 |
| phosphatase | | | | | |
| Diacylglycerol kinase | EC 2.7.1.174 | DGK1 | YOR311C | YALI0F19052g | ANZ74498.1 |
| Enzymes involved in | | | | | |
| lipid catabolism | | | | | |
| Cholesterol esterase/ | EC 3.1.1.13 | TGL1 | YKL140W | YALI0E32035g | ANZ73240.1 |
| TAG lipase | | | | | ANZ74310.1 |
| TAG lipase | EC 3.1.1.3 | TGL3 | YMR313C | YALI0D17534g | ANZ77507.1 |
| TAG lipase | EC 3.1.1.3 | TGL4 | YKR089C | YALI0F10010g | none |
| TAG lipase | EC 3.1.1.3 | TGL5 | YOR081C | none | ANZ75256.1 |
| Phospholipase B | EC 3.1.1.5 | PLB2 | YMR006C | none | ANZ75819.1 |
| Phospholipase B | EC 3.1.1.5 | PLB1 | YMR008C | YALI0E16060g | ANZ75299.1 |
| Phospholipase D | EC 3.1.4.4 | SPO14 | YKR031C | YALI0E18898g | ANZ76336.1 |
| Peroxisome biogenesis | | PEX10 | YDR265W | YALI0C01023g | ANZ77203.1 |
| factor 10 | | | | | |
| Acyl-CoA oxidase | EC 1.3.3.6 | POX1 | YGL205W | YALI0E32835g | ANZ76334.1 |
| Acyl-CoA oxidase | EC 1.3.3.6 | POX2 | YKR009C | YALI0F10857g | none |
| Acyl-CoA oxidase | EC 1.3.3.6 | POX3 | YIL160C | YALI0D24750g | none |
| Acyl-CoA oxidase | EC 1.3.3.6 | POX4 | none | YALI0E27654g | none |
| Acyl-CoA oxidase | EC 1.3.3.6 | POX5 | none | YALI0C23859g | none |
| Acyl-CoA oxidase | EC 1.3.3.6 | POX6 | none | YALI0E06567g | none |
| Protein kinase | EC 2.7.11.1 | SNF1 | YDR477W | YALI0D02101g | ANZ75125.1 |
| Multifunctional- | EC 4.2.1.74 | MFE1 | YKR009C | YALI0E15378g | ANZ74935.1 |
| oxidation protein | | | | | |
| Peroxisomal oxoacyl | EC 2.3.1.16 | POT1 | YIL160C | YALI0E18568g | ANZ75015.1 |
| thiolase | | | | | |
| Regulators of lipid | | | | | |
| synthesis | | | | | |
| Associated with Sit4 | EC 3.1.3.16 | SAP190 | YKR028W | YALI0F11869g | ANZ77566.1 |
| protein phosphatase | | | | | |
| Phosphoinositide | EC 2.7.1.67 & | TOR2 | YKL203C | YALI0F07084g | ANZ75729.1 |
| 3-kinase-related | EC 2.7.11.1 | | | | |
| protein kinase | | | | | |
| Phospholipid | | OPI1 | YHL020C | YALI0C14784g | ANZ73581.1 |
| synthesis | | | | | |
| regulatory protein | | | | | |

| | Gene in | | |
|---|---|---|---|
| | Mortierella | Gene in | |
| Enzyme or protein | alpina | E. coli | References |
| Fatty acid synthesis | | | |
| and TAG production | | | |
| Acetyl-CoA | KAF9286715.1 | P27550 | DeVirgilio, 1992 |
| synthetase | | | |
| Acetyl-CoA | none | none | Hiesinger et al., 1997 |
| synthetase | | | |
| ATP-citrate lyase | KAF9948114.1 | P75726 | Dulermo, 2015; |
| subunit | | | Feng et al., 2015 |
| ATP-citrate lyase | KAF9929232.1 | P0A9I1 | Dulermo, 2015; |
| subunit | | | Feng et al., 2015 |
| Acetyl-CoA | KAF9288230.1 | P0ABD5, | Al-Feel et al., 1992; |
| carboxylase | | P0ABD8, | Feng et al., 2015 |
| | | P24182, | |
| | | P0A9Q5 | |
| Fatty acid synthase | KAF9285532.1 | P0AAI9, | Janßen et al., 2014 |
| subunit beta | | P0A6R0, | |
| | | P0AEK2, | |
| | | P0A6Q3, | |
| | | P0A6Q6, | |
| | | P0AEK4, | |
| | | P0A953, | |
| | | P0AAI5 | |
| Fatty acid synthase | KAF9936935.1 | none | Schüller et al., 1992 |
| subunit alpha | | | |
| Fatty acid elongase | BAI40363.1 | none | Schneiter et al., 2000 |
| Fatty acid elongase | BAH02594.1 | none | Oh et al., 1997 |
| Fatty acid elongase | ADE06662.1 | none | Oh et al., 1997 |
| Δ5 Fatty acid | AAC39508.1 | none | Michaelson. 1998 |
| desaturase | | | |
| Δ9 Fatty acid | CAB38178.1 | MHO05803.1 | Stukey et al., 1989; |
| desaturase | | | Wongwathanarat et |
| | | | al., 1999 |
| Δ12 Fatty acid | Q9Y8H5.2 | none | Kassab et al., 2019 |
| desaturase | | | |
| Δ15/ω3 Fatty | Q59J82.1 | none | Kassab et al., 2019 |
| acid | | | |
| desaturase | | | |
| Glycerol kinase | KAF9275064.1 | P0A6F3 | Beopoulos et al., 2008 |
| G3P dehydrogenase | ALM55659.1 | P13035 | Beopoulos et al., 2008 |
| (NAD) | | | |
| G3P dehydrogenase | none | none | Beopoulos et al., 2008 |
| (NAD) | | | |
| G3P dehydrogenase | KAF9947304.1 | P0A9C0, | Beopoulos et al., 2008 |
| (mitochondria) | | P13033, | |
| | | P0A996 | |
| Glycerophosphate | KAF9941189.1 | P0A7A7 | Beopoulos et al., 2008; |
| acyltransferase | | | Janßen et al., 2014 |
| (GPAT) | | | |
| Glycerophosphate | KAF9964849.1 | P60782 | Beopoulos et al., 2008; |
| acyltransferase | | | Janßen et al., 2014 |
| (GPAT) | | | |
| 1-Acyl-sn- | AED33305 | P26647 | Beopoulos et al., 2008; |
| glycerophosphate | | | Janßen et al., 2014 |
| acyltransferase | | | |
| (LPAAT) | | | |
| 1-Acyl-sn- | KAF9279687.1 | none | Janßen et al., 2014 |
| glycerophosphate | | | |
| acyltransferase | | | |
| (LPAAT) | | | |
| 1-Acyl-sn- | KAF9967416.1 | none | Janßen et al., 2014 |
| glycerophosphate | | | |
| acyltransferase | | | |
| (LPAAT) | | | |
| Phosphatidic acid | none | none | Adeyo et al., 2011 |
| phosphatase (PAP) - | | | |
| phosphohydrolase | | | |
| Phosphatidic acid | KAF9924983.1 | P0A924 | Pascual et al., 2013 |
| phosphatase (PAP) | | | |
| Diacylglycerol | ATQ62217.1 | none | Beopoulos et al., 2008; |
| acyltransferase | | | Janßen et al., 2014 |
| (DGAT) | | | |
| Diacylglycerol | AQX34626.1 | none | Janßen et al., 2014 |
| acyltransferase | | | |
| (DGAT) | | | |
| Phospholipid:diacyl- | KAF9951579.1 | none | Beopoulos et al., 2008 |
| glycerol | | | |
| acyltransferase | | | |
| (PDAT) | | | |
| Acyl-CoA:sterol | KAF9276817.1 | none | Beopoulos et al., 2008 |
| acyltransferase | | | |
| Acyl-CoA:sterol | none | none | Beopoulos et al., 2008 |
| acyltransferase | | | |
| Cardiolipin synthase | KAF9948624.1 | P0ABF8 | Džugasová et al., 1998 |
| Fatty acyl-CoA | KAF9940771.1 | P69451, | Li et al., 2007; |
| synthetase | | P38135 | Dabirian et al., 2019 |
| Enzymes involved in | | | |
| PL synthesis | | | |
| Inositol-3-phosphate | KAF9950016.1 | P11986 | Feng et al., 2015 |
| synthase | | | |
| Phosphatidate | KAF9956759.1 | P0ABG1 | Shen et al., 1996; |
| cytidylyltransferase | | | Shen and Dowhan, 1997 |
| CDP-DAG synthase | KAF9291471.1 | none | Tamura et al., 2013 |
| Phosphatidylinositol | KAF9953551.1 | none | Fischl et al., 1986; |
| synthase | | | Jani and Lopes, 2009 |
| Phosphatidylserine | KAF9960908.1 | P23830 | Delhaize et al., 1999; |
| synthase | | | Han et al., 2017 |
| Phosphatidylserine | KAF9951850.1 | P0A8K1 | Clancey et al., 1993; |
| decarboxylase | | | Gsell et al., 2013 |
| Phosphatidylserine | KAF9963219.1 | none | Trotter and Voelker, 1995 |
| decarboxylase | | | |
| Phosphatidyl- | KAF9280886.1 | none | Summers et al., 1988; |
| ethanolamine | | | Kodaki et al., 1989 |
| methyltransferase | | | |
| Phospholipid | KAF9947517.1 | none | Kodaki et al., 1987; |
| methyltransferase | | | McGraw and Henry, 1989 |
| Phosphatidylinositol | KAF9289337.1 | none | Ren et al., 2014 |
| transfer protein | | | |
| Phosphatidylinositol/ | KAF9282151.1 | none | Bankaitis et al., 2007; |
| phosphatidylcholine | | | Tripathi et al., 2019 |
| transfer protein | | | |
| Phosphatidylinositol | KAF9968193.1 | none | Tani et al., 2014 |
| phosphatase | | | |
| Diacylglycerol kinase | none | P0ABN1 | Han et al., 2008; |
| | | | Fakas et al., 2011 |
| Enzymes involved in | | | |
| lipid catabolism | | | |
| Cholesterol esterase/ | KAF9965658.1 | none | Beopoulos et al., 2008 |
| TAG lipase | | | |
| TAG lipase | KAF9957419.1 | none | Beopoulos et al., 2008 |
| TAG lipase | none | none | Beopoulos et al., 2008; |
| | | | Klein et al., 2016 |
| TAG lipase | KAF9949630.1 | none | Klein et al., 2016 |
| Phospholipase B | KAF9960596.1 | none | Fyrst et al., 1999; |
| | | | Ferreira et al., 2018 |
| Phospholipase B | KAF9290270.1 | P0ADA1 | Lee et a.1, 1994; |
| | | | Ferreira et al., 2018 |
| Phospholipase D | KAF9940421.1 | none | Sreenivas et al., 1998 |
| Peroxisome biogenesis | KAF9964377.1 | none | Williams et al., 2008 |
| factor 10 | | | |
| Acyl-CoA oxidase | KAF9281751.1 | none | Beopoulos et al., 2008 |
| Acyl-CoA oxidase | KAF9927487.1 | none | Beopoulos et al., 2008 |
| Acyl-CoA oxidase | KAF9966479.1 | none | Beopoulos et al., 2008 |
| Acyl-CoA oxidase | KAF9928534.1 | none | Beopoulos et al., 2008 |
| Acyl-CoA oxidase | KAF9281340.1 | none | Beopoulos et al., 2008 |
| Acyl-CoA oxidase | none | none | Beopoulos et al., 2008 |
| Protein kinase | KAF9966796.1 | none | Feng et al., 2015 |
| Multifunctional- | KAF9928572.1 | none | Beopoulos et al., 2008 |
| oxidation protein | | | |
| Peroxisomal oxoacyl | KAF9274906.1 | P21151, | Beopoulos et al., 2008; |
| thiolase | | P76503 | Feng et al., 2015 |
| Regulators of lipid | | | |
| synthesis | | | |
| Associated with Sit4 | KAF9941704.1 | none | Luke et al., 1996 |
| protein phosphatase | | | |
| Phosphoinositide | KAF9954459.1 | P23874 | Helliwell et al., 1998 |
| 3-kinase-related | | | |
| protein kinase | | | |
| Phospholipid | KAF9965110.1 | none | Sreenivas et al., |
| synthesis | | | 2001; Sreenivas and |
| regulatory protein | | | and Carman, 2003 |

### Synthesis of Phospholipids in Microbes

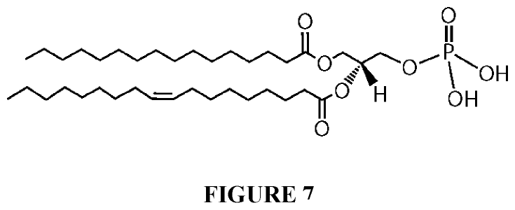

As a primary structural component of biological membranes, phospholipids play important roles in cell morphology and organelle function and some also act as secondary messengers. Phospholipids are amphipathic molecules that have a glycerol backbone esterified to a phosphate head group and two fatty acids (FIG. 7). Due to their charged headgroup at neutral pH, they are polar lipids, showing some solubility in solvents such as ethanol in addition to solvents such as chloroform. The most common fatty acids esterified to the glycerophosphate backbone of phospholipids in eukaryotic microbes such as S. cerevisiae include palmitic acid (C16:0), palmitoleic acid (C16:1), stearic acid (C18:0) and oleic acid (C18:1) (Carman and Gil-Soo, 2011). The major phospholipids found in total cell extracts from S. cerevisiae are phosphatidylcholine (PC), phosphatidylethanolamine (PE), phosphatidylinositol (PI), and phosphatidylserine (PS). Phosphatidyl glycerol (PG) and cardiolipin (CL) are minor phospholipids in total S. cerevisiae cell extracts but are the major phospholipids of mitochondrial lipids (Zhang et al., 2014). Other yeasts such as Y. lipolytica and Schizosaccharomyces pombe have a similar phospholipid make up (Fernandez et al., 1986, Fakas 2017). In contrast, the phospholipid composition of prokaryotes such as Escherichia coli is primarily comprised of PE, PG and CL and these phospholipids mainly contain the fatty acids 16:0, 16:1 and 18:1411 (De Siervo 1969). E. coli and many other bacteria lack PC.

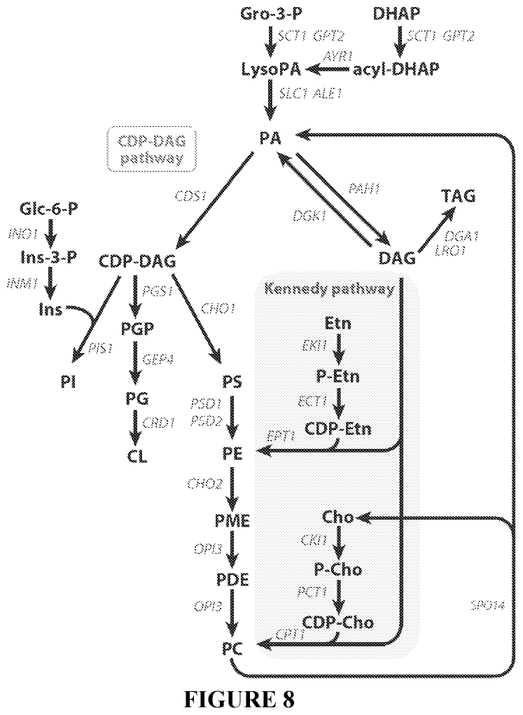

The enzymes involved in the synthesis of phospholipids in microbes and the corresponding genes are listed in Table 1 and a schematic of the pathways for phospholipid synthesis is shown in FIG. 8. The enzymes and genes involved in phospholipid synthesis in yeast have been characterised in detail in S. cerevisiae (Carman and Zeimetz, 1996). The specific synthesis of phospholipids begins with the synthesis of the phospholipid phosphatidic acid (PA), which is produced from glycerol-3-phosphate or dihydroxyacetone phosphate after fatty acyl coenzyme A (CoA)-dependent reactions that are catalyzed by glycerol-3-phosphate acyltransferases and the lysophospholipid acyltransferases (Athenstaedt and Daum, 1997; Athenstaedt et al., 1999; Zheng and Zou, 2001). All major phospholipid classes in S. cerevisiae are synthesized from a common precursor: cytidine diphosphate diacylglycerol (CDP-DAG). CDP-DAG is synthesized in a reaction catalyzed by CDP-DAG synthase, which converts PA to CDP-DAG using cytidine triphosphate (CTP) as the CDP donor (Carter and Kennedy 1966; Shen et al., 1996). CDP-DAG is the key intermediate for the synthesis of all of the major and minor phospholipids in S. cerevisiae as in all other yeasts. In one reaction, CDP-DAG donates its phosphatidyl moiety to inositol to form PI in the reaction catalyzed by PI synthase (Nikawa and Yamashita, 1984). The inositol used in this reaction can be derived from glucose-6-phosphate via the reactions catalyzed by inositol-3-phosphate synthase (Klig and Henry, 1984; Dean-Johnson and Henry, 1989) and inositol-3-phosphate phosphatase (Murray and Greenberg, 2000). Inositol used in the synthesis of PI can also be utilised from exogenously supplied inositol in the media by inositol permeases. CDP-DAG may also donate its phosphatidyl moiety to glycerol-3-phosphate to form phosphatidylglycerophosphate (PGP) in the reaction catalyzed by PGP synthase (Chang et al., 1998). PGP is then dephosphorylated to PG by PGP phosphatase (Osman et al., 2010). The cardiolipin (CL) synthase catalyzes the reaction between PG and another molecule of CDP-DAG to generate CL (Chang et al., 1998). The final enzyme that utilizes CDP-DAG is the PS synthase (Letts et al., 1983) which catalyzes the formation of PS by displacement of CMP from CDP-DAG with serine (Kanfer and Kennedy, 1964). PS is then decarboxylated to PE by PS decarboxylase enzymes (Trotter et al., 1993). PE is then converted to PC by the three-step S-adenosyl methionine (AdoMet)-dependent methylation reactions, whereby the first methylation reaction is catalyzed by the PE methyltransferase and the last two methylation reactions are catalyzed by the phospholipid methyltransferase (Kodaki and Yamashita, 1987).

PE and PC can also be synthesised from exogenously supplied ethanolamine and choline by the CDP-ethanolamine and CDP-choline branches of the Kennedy pathway (Nikawa et al., 1987). The exogenously supplied ethanolamine and choline are phosphorylated by ethanolamine kinase and choline kinase with ATP to form phosphoethanolamine and phosphocholine, respectively (Kim et al., 1999; Hosaka et al., 1989). These intermediates are then activated with CTP to form CDP-ethanolamine and CDP-choline, respectively, by phosphoethanolamine cytidylyltransferase and phosphocholine cytidylyltransferase (Min-Seok et al., 1996; Tsukagoshi et al., 1987). Ethanolamine phosphotransferase and choline phosphotransferase then convert CDP-ethanolamine and CDP-choline in a reaction with DAG to form PE and PC (Hjelmstad and Bell 1988; Hjelmstad and Bell, 1991). The CTP required for the synthesis of CDP-DAG, CDP-ethanolamine, and CDP-choline is derived from UTP by the action of CTP synthetase enzymes. The DAG used for the synthesis of PE and PC via the Kennedy pathway is derived from PA by the PAH1-encoded PA phosphatase (Han et al., 2006). The DAG generated in the PA phosphatase reaction may be converted back to PA by DAG kinase (Han et al., 2008a; Han et al., 2008b) or used for the synthesis of the neutral lipid TAG by acyltransferase enzymes encoded by DGA1 and LRO1. In addition, additional acyltransferase enzymes involved in the synthesis of ergosterol esters can also acylate DAG to form TAG.

The Kennedy pathway plays a critical role in the synthesis of PE and PC when the enzymes in the CDP-DAG pathway are non-functional or defective (Carman and Henry, 1999; Greenberg and Lopes, 1996). For example, a mutant deficient in the three-step methylation of PE requires choline supplementation for growth and synthesizes PC via the CDP-choline branch of the Kennedy pathway. Mutants deficient in the synthesis of PS or PE can synthesize PC if they are supplemented with ethanolamine or choline, respectively. The ethanolamine is incorporated into PE via the CDP-ethanolamine branch of the Kennedy pathway, and the PE is subsequently methylated to form PC. Mutants defective in the CDP-DAG pathway can also synthesize PE or PC when they are supplemented with lysoPE, lysoPC, or PC with short acyl chains. LysoPE and lysoPC transported into the cell are acylated to PE and PC, respectively, by the lysophospholipid acyltransferase, which also utilizes lysoPA as a substrate. In addition, Kennedy pathway mutants defective in both the CDP-choline and CDP-ethanolamine branches can synthesize PC only by the CDP-DAG pathway. However, unlike the CDP-DAG pathway mutants the Kennedy pathway mutants do not exhibit any auxotrophic requirements and have an essentially normal complement of phospholipids.

Evidence supports that the CDP-DAG pathway is mainly responsible for the synthesis of PE and PC when cells are grown in the absence of ethanolamine and choline (Carman and Henry 1989). However, the Kennedy pathway can contribute to the synthesis of PE and PC when these precursors are not supplemented in the culture medium. For example, the PC synthesized by way of the CDP-DAG pathway is constantly hydrolyzed to choline and PA by a phospholipase D. The choline can then be incorporated back into PC via the CDP-choline branch of the Kennedy pathway, and the PA is converted to other phospholipids via the intermediates CDP-DAG and DAG.

The details provided above for S. cerevisiae phospholipid synthesis and the gene and enzymes involved are found to be also true for the oleaginous yeast Yarrowia lipolytica. Another common yeast, S. pombe, uses pathways for PL biosynthesis that are highly similar to those of S. cerevisiae. There is, however, one major difference between S. pombe and S. cerevisiae. S. pombe is a natural inositol auxotroph; it cannot grow in the absence of inositol due to the inability to form L-myoinositol 3-phosphate from its precursor glucose 6-phosphate. As a result, the PI content of S. pombe cells is strongly dependent on the concentration of inositol in the growth medium. Inositol auxotrophy of S. pombe is due to the absence of inositol-3-phosphate synthase, encoded by the INO1 gene in S. cerevisiae, as evidenced by the observation that expression of Pichia pastoris inositol-3-phosphate synthase in S. pombe can convert this natural inositol auxotroph to the inositol prototroph.

Phospholipids in E. coli and other Gram-negative bacteria are used in the construction of the inner and outer membranes. E. coli possesses only three major phospholipid species in its membranes, PE which comprises the bulk of the phospholipids (75%), with PG and CL forming the remainder, 15-20% and 5-10%, respectively. Bacterial phospholipid synthesis begins with the acylation of glycerol 3-phosphate (G3P), forming lysophosphatidic acid (lysoPA). This detergent-like intermediate undergoes a second acylation, forming phosphatidic acid (PA) which is the key precursor for bacterial phospholipids. The major PL of E. coli are synthesised from PA by the enzymes of the CDP-DAG pathway as described for S. cerevisiae. In summary, the acyltransfer module deposits PA in the membrane, where it is activated to CDP-DAG by CDP-DAG synthase. This intermediate is used for both PE synthesis via PS synthase and PS decarboxylase (Psd). PG is formed from the same intermediate by PGP synthase and the phosphorylated intermediate is dephosphorylated by PGP phosphatase. Finally, CL is produced by the condensation of two PG molecules by CL synthase.

### Microbial Cells

A wide variety of different microbial cells can be used in the present invention. In an embodiment the microbial cells exist as single celled organisms, however such cells may clump together. Examples of microbial cells of the invention include bacterial cells and eukaryotic cells such as fungal cells and algal cells. Eukaryotic microbes are preferred over bacterial (prokaryotic) microbes. As used herein, the terms “microbial cell”, “microbe” and “microorganism” mean the same thing.

In an embodiment, the microbial cells are suitable for fermentation, although they can also be cultured under ambient oxygen concentrations. In another embodiment, the microbial cells are oleaginous cells, preferably an oleaginous eukaryotic microbe, or preferably derived from a progenitor oleaginous microbe such as a progenitor eukaryotic oleaginous microbe. In another embodiment, microbial cells are heterotrophic cells, preferably a heterotrophic eukaryotic microbe. The microbial cells preferably have at least two of these, more preferably are characterised by all of these features.

In an embodiment, the cells of the invention are yeast cells. Examples of yeast cells useful for the invention include, but are not limited to, Saccharomyces sp. such as Saccharomyces cerevisiae, Yarrowia sp. such as Yarrowia lipolytica, Pichia sp. such as Pichia pastoris, Candida sp. such as Candida rugosa, Aspergillus sp. such as Aspergillus niger, Cryptococcus sp. such as Cryptococcus curvatus, Lipomyces sp. such as Lipomyces starkeyi, Rhodosporidium sp. such as Rhodosporidium toruloides, Rhodotorula sp. such as Rhodotorula glutinis and Trichosporon sp. such as Trichosporon fermentans.

In an embodiment, the fungal cells are mold cells. Examples of mold cells useful for the invention include, but are not limited to, Cunninghamella sp. such as Cunninghamella echinulate, Mortierella sp. such as Mortierella alpina, Mortierella elongata and Mortierella exigua, Mucorales sp. such as Mucorales fungi and Trichoderma sp. such as Trichoderma harzianum.

In an embodiment, the cells are bacterial cells. Examples of bacterial cells useful for the invention include, but are not limited to, Acinetobacter such as Acinetobacter baylyi, Alcanivorax sp. such as Alcanivorax borkumensis, Gordonia sp. such as DG, Mycobacterium sp. such as Mycobacterium tuberculosis, Nocardia sp. such as Nocardia globerula, Rhodococcus sp. such as Rhodococcus opacus, and Streptomyces sp. such Streptomyces coelicolor.

In an embodiment, the cells are algal cells such as microalgal, or Bacillariophyceae, cells. Examples of algal cells useful for the invention include, but are not limited to, Prototheca sp. such as Prototheca moriformis, Thraustochytrium spp., Chlorella sp. such as Chlorella protothecoides, Chlorella vulgaris or Chlorella ellipsoidea, Schizochytrium sp. such as Schizochytrium strain FCC-1324, Dunaliella sp., Haematococcus sp. such as Haematococcus pluvialis, Neochloris sp. such as Neochloris oleabundans such as strain UTEX #1185, Pseudochlorococcum sp., Scenedesmus sp. such as Scenedesmus obliquus, Tetraselmis sp. such as Tetraselmis chui or Tetraselmis tetrathele, Chaetoceros sp. such as Chaetoceros calcitrans, Chaetoceros gracilis or Chaetoceros muelleri, Nitzschia sp. such as Nitzschia cf. pusilla, Phaeodactylum sp. such as Phaeodactylum tricornutum, Skeletonema sp. such as strain CS 252, Thalassiosira sp. such as Thalassiosira pseudonana, Crypthecodinium sp. such as Crypthecodinium cohnii, Isochrysis sp. such as Isochrysis zhangjiangensis, Nannochloropsis sp. such as Nannochloropsis oculata such as strain NCTU-3, Pavlova sp. such as Pavlova salina, Rhodomonas sp. and Thalassiosira sp. such as Thalassiosira weissflogii.

In one embodiment, the cell is a genetically modified microbe.

In embodiments, the genetically modified microbe has one or more genetic modification(s) which provide for
(i) synthesis of, or increased synthesis of, one or more ω6 fatty acids in the microbe,
(ii) an increase in total fatty acid synthesis and/or accumulation in the microbe,
(iii) an increase in total polar lipid synthesis and/or accumulation in the microbe,
(iv) a decrease in triacylglycerol (TAG) synthesis and/or accumulation in the microbe, or an increase in TAG catabolism in the microbe, preferably an increase in TAG lipase activity, or
(v) a reduction in catabolism of total fatty acids in the microbe,
or any combination thereof.

The genetic modification(s) may include introduction of an exogenous polynucleotide, a mutation or a deletion of a gene or regulatory sequence, or any other known genetic modification. Suitable techniques for genetically modifying microbes are described herein.

In one embodiment, the genetic modification(s) provide for at least two of (i) to (v) above, preferably (iv) and (v), or (i), (iv) and (v).

In one embodiment, the genetic modification(s) are selected from the group consisting of:
(i) one or more exogenous polynucleotide(s) encoding a Δ12 desaturase, Δ6 desaturase, Δ6 elongase, Δ9 elongase, Δ8 desaturase, Δ5 desaturase, Δ5 elongase, Δ4 desaturase or any combination thereof;
(ii) one or more genetic modification(s) that result in an increased expression and/or activity of acetyl-CoA synthetase, ATP-citrate lyase, acetyl-CoA carboxylase, fatty acid synthase subunit beta or fatty acid synthase subunit alpha, or any combination thereof;
(iii) one or more genetic modification(s) that result in an increased expression and/or activity of CDP-DAG synthase, phosphatidylinositol synthase, phosphatidylserine synthase, phosphatidylserine decarboxylase, phosphatidylethanolamine methyltransferase, phospholipid methyltransferase, phosphatidylinositol transfer protein, phosphatidylinositol/phosphatidylcholine transfer protein, phosphatidylinositol phosphatase, phosphatidate cytidylytransferase, or diacylglycerol kinase (DGK);
(iv) one or more genetic modification(s) that result in a decrease in expression and/or activity of DGAT1, DGAT2, LRO1, ARE1 or ARE2; and
(v) one or more genetic modification(s) that result in a decreased expression and/or activity of cholesterol esterase/TAG lipase, TAG lipase, phospholipase B, phospholipase D, acyl-CoA oxidase, acyl-CoA oxidase 2, acyl-CoA oxidase 3, acyl-CoA oxidase 5, multifunctional-oxidation protein or peroxisomal oxoacyl thiolase.

Preferred combinations of enzymes encoded by the polynucleotides of (i) according to the Δ6 desaturase pathway are (a) a Δ12 desaturase and a Δ6 desaturase to produce GLA, (b) a Δ12 desaturase, a Δ6 desaturase and a Δ6 elongase to produce GLA and DGLA, (c) a Δ12 desaturase, a Δ6 desaturase, a Δ6 elongase and a Δ5 desaturase to produce GLA, DGLA and ARA, (d) a Δ12 desaturase, a Δ6 desaturase, a Δ6 elongase, a Δ5 desaturase and a Δ5 elongase to produce GLA, DGLA, ARA and DTA, and (e) a Δ12 desaturase, a Δ6 desaturase, a Δ6 elongase, a Δ5 desaturase, a Δ5 elongase and a Δ4 desaturase to produce GLA, DGLA, ARA, DTA and DPAω6. Preferred combinations of enzymes encoded by the polynucleotides of (i) according to the Δ9 elongase pathway are (f) a Δ12 desaturase and a Δ9 elongase to produce EDA, (g) a Δ12 desaturase, a Δ9 elongase and a Δ8 desaturase to produce EDA and DGLA, (h) a Δ12 desaturase, a Δ9 elongase, a Δ8 desaturase and a Δ5 desaturase to produce EDA, DGLA and ARA, (i) a Δ12 desaturase, a Δ9 elongase, a Δ8 desaturase, a Δ5 desaturase and a Δ5 elongase to produce EDA, DGLA, ARA and DTA, and (j) a Δ12 desaturase, a Δ9 elongase, a Δ8 desaturase, a Δ5 desaturase, a Δ5 elongase and a Δ4 desaturase to produce EDA, DGLA, ARA, DTA and DPAω6. In each of combinations (a) to (j), the Δ12 desaturase can be omitted if the microbial cell has an endogenous Δ12 desaturase which converts oleic acid to LA with sufficient activity to enable production of sufficient ω6 fatty acids. The person of skill in the art can readily determine whether an exogenous Δ12 desaturase should be used.

Preferred combinations of enzymes encoded by the polynucleotides of (iii) are (a) one or more or all three of diacylglycerol kinase, phosphatidate cytidylytransferase and phosphatidylserine synthase, (b) diacylglycerol kinase, phosphatidate cytidylytransferase, phosphatidylserine synthase and phosphatidylserine decarboxylase, (c) phosphatidate cytidylytransferase, phosphatidylserine synthase and phosphatidylserine decarboxylase, and (d) phosphatidylserine synthase and phosphatidylserine decarboxylase. To produce more PC, polynucleotides encoding phosphatidylethanolamine methyltransferase, or phosphatidylethanolamine methyltransferase and phospholipid methyltransferase, can be added to any of the combinations (a) to (d), or used on their own.

Preferred combinations of genetic modifications of (iv) are those that reduce the activity of DGAT1 and LRO1, or all three of DGAT1, DGAT2 and LRO1.

More preferred embodiments of the preferred embodiments described above include an addition of a genetic modification which reduces the activity of a regulator of lipid synthesis, for example null mutations in any one of the genes SAP190, TOR2 or most preferably OPI1.

In one embodiment, the genetically modified microbe comprises one or more genetic modification(s) which increase the amount of at least two phospholipids selected from the group consisting of PC, PE, PS and PI relative to a corresponding wild-type microbe, wherein each amount is expressed as a percentage of the total polar lipid content. The genetic modifications to achieve this include those in the preceding paragraphs.

In embodiments, the at least two phospholipids are PC and PE, PC and PS, or PC and PI, or wherein PC and PE, PC and PS, or PC and PI are present in an altered ratio relative to polar lipid from the corresponding wild-type microbe.

In another aspect, the present invention provides microbial cells comprising lipid of the invention. The microbial cells may be in suspension for example an aqueous suspension, frozen, dried or any other suitable form. The microbial cells may be alive or dead, or a mix of living and dead cells, for example at least 99% of the cells being dead. The cells may have been heat-treated in order to render them incapable of replicating.

In embodiments, the microbial cells comprise or consist of eukaryotic cells, fungal cells, bacterial cells or algal cells, living microbial cells, dead microbial cells, or any mixture thereof. In embodiments, the microbial cells are one or more or all of (i) suitable for fermentation, (ii) oleaginous cells, (iii) non-oleaginous cells, preferably non-oleaginous cells derived from oleaginous cells by genetic modification, and (iv) heterotrophic cells.

In embodiments, the microbial cells are yeast cells. Examples include, but are not limited to Saccharomyces cerevisiae, Yarrowia lipolytica, Pichia pastoris, Trichoderma spp., Candida rugose, Aspergillus niger, Crypthecodinium cohnii and any mixture thereof.

In one embodiment, the yeast cells are selected from the group consisting of Saccharomyces cerevisiae, Yarrowia lipolytica, Pichia pastoris and any mixture thereof.

In an embodiment, the microbial cells comprise algal cells selected from the group consisting of Prototheca moriformis, Thraustochytrium spp., Chlorella protothecoides, Schizochytrium sp. such as strain FCC-1324, and any mixture thereof.

In an embodiment, the fungal cells are of a filamentous fungus or mold species, for example Mortierella sp. such as Mortierella alpina or Mortierella elongata. In an embodiment, the fungal cells are from the Genus Mucor, for example from the species Mucor hiemalis. Examples of Mortierella sp. include those of the present invention.

In an embodiment, the microbial cells are microbial cells other than Mortierella alpina.

In an embodiment, the microbial cells comprise a genetic modification resulting in an increase in production of ω6 fatty acids in polar lipid. In one embodiment, the microbial cells comprise one or more of the genetic modifications listed above in relation to the lipid of the invention.

In one embodiment, the microbial cells comprise a genetic modification resulting in a reduction in endogenous Δ12 desaturase expression and/or activity. In one embodiment, the genetic modification is a mutation in a gene encoding the endogenous Δ12 desaturase, preferably a null mutation of a FAD2 gene. In one embodiment, the null mutation is a gene deletion. Surprisingly, the present inventors observed that the amount of ω6 fatty acid such as ARA incorporated into the polar lipid fraction in yeast was increased in a fad2 null mutant compared to the corresponding wild-type strain, when the ω6 fatty acid was supplied to the culture medium. In an embodiment, the amount of ω6 fatty acid produced endogenously in the fad2 mutant microbial cell is increased relative to a corresponding FAD2 wild-type cell.

In one embodiment, the microbial cells comprise one or more genetic modification(s) resulting in reduction of triacylglycerol (TAG) synthesis. In an embodiment, the one or more genetic modification(s) comprise one or more mutations which each reduce the expression and/or activity of a DGA1, DGA2, LRO1 or ARE1 gene, preferably comprising a null mutation of, any one or more or all of the DGA1, DGA2, LRO1 and ARE1 genes. In one embodiment, the null mutation is a deletion of at least part of the gene.

In embodiments, the microbial cells comprise mutations which reduce the expression and/or activity, preferably null mutations, of:
a) at least DGA1 and DGA2;
b) at least DGA1 and LRO1;
c) at least DGA1, DGA2 and LRO1; or
d) at least DGA1, DGA2, LRO1 and ARE1.

In one embodiment, the microbial cells comprise one or more exogenous polynucleotide(s) encoding one or more desaturase(s) and/or one or more elongase(s).

In embodiments, the microbial cells comprise one or more exogenous polynucleotide(s) encoding at least:
a) a Δ12 desaturase;
b) a Δ5 elongase;
c) a Δ5 elongase and a Δ4 desaturase;
d) a Δ6 desaturase and, optionally, a Δ12 desaturase;
e) a Δ9 elongase and, optionally, a Δ12 desaturase;
f) a Δ6 desaturase, a Δ6 elongase and, optionally, a Δ12 desaturase;
g) a Δ9 elongase, a Δ8 desaturase and, optionally, a Δ12 desaturase;
h) a Δ6 desaturase, a Δ6 elongase, a Δ5 desaturase and, optionally, a Δ12 desaturase;
i) a Δ9 elongase, a Δ8 desaturase, a Δ5 desaturase and, optionally, a Δ12 desaturase;
j) a Δ6 desaturase, a Δ6 elongase, a Δ5 desaturase, a Δ5 elongase and, optionally, a Δ12 desaturase;
k) a Δ9 elongase, a Δ8 desaturase, a Δ5 desaturase, a Δ5 elongase and, optionally, a Δ12 desaturase;
l) a Δ6 desaturase, a Δ6 elongase, a Δ5 desaturase, a Δ5 elongase, a Δ4 desaturase and, optionally, a Δ12 desaturase; or
m) a Δ9 elongase, a Δ8 desaturase, a Δ5 desaturase, a Δ5 elongase, a Δ4 desaturase and, optionally, a Δ12 desaturase,
wherein each polynucleotide is operably linked to one or more promoters that are capable of directing expression of said polynucleotides in the microbial cells.

In embodiments, the microbial cells comprise one or more exogenous polynucleotide(s) encoding a Δ6 desaturase, a Δ6 elongase, a Δ5 desaturase and, optionally, a Δ12 desaturase, wherein each polynucleotide is operably linked to one or more promoters that are capable of directing expression of said polynucleotides in the microbial cell. The microbial cells may comprise two or more Δ6 desaturase genes, two or more Δ6 elongase genes, two or more Δ5 desaturase genes, and/or two or more Δ12 desaturase genes, in each case encoding either the same or different enzymes.

In embodiments, the microbial cells comprise one or more exogenous polynucleotide(s) encoding a Δ9 elongase, a Δ8 desaturase, a Δ5 desaturase and, optionally, a Δ12 desaturase, wherein each polynucleotide is operably linked to one or more promoters that are capable of directing expression of said polynucleotides in the microbial cell. The microbial cells may further comprise one or more exogenous polynucleotide(s) encoding a Δ6 desaturase and a Δ6 elongase. The microbial cells may comprise two or more Δ8 desaturase genes, two or more Δ9 elongase genes, two or more Δ5 desaturase genes, and/or two or more Δ12 desaturase genes, in each case encoding either the same or different enzymes.

In an embodiment, the one or more exogenous polynucleotides are integrated into the genome of the cell. In an embodiment, the exogenous polynucleotides are integrated into a single site in the microbial cell genome. In an alternative embodiment, the exogenous polynucleotides are not integrated into a single site in the microbial cell genome but instead one or more are integrated at one site and one or more other polynucleotides are integrated at another site in the genome. The polynucleotides may be integrated at three or more sites in the genome.

In one embodiment, one or more or all of the desaturases and/or elongases have greater activity on an ω6 fatty acid when compared to a corresponding ω3 fatty acid.

The desaturases above may act on CoA-bound or PC-bound substrates or both. In one embodiment, one or more or all of the desaturases, preferably the Δ6-desaturase and/or the Δ5-desaturase, and/or the Δ12 desaturase, have greater activity on an acyl-CoA substrate than a corresponding acyl-PC substrate.

In embodiments, the Δ12 desaturase comprises amino acids having a sequence set forth as any one of SEQ ID NOs: 1 to 4, or an amino acid sequence which is at least 60%, at least 70%, at least 80%, at least 90% or at least 95% identical to any one or more of SEQ ID NOs: 1 to 4.

In embodiments, the Δ12 desaturase comprises amino acids having a sequence set forth as SEQ ID NO:1, or an amino acid sequence which is at least 60%, at least 70%, at least 80%, at least 90% or at least 95% identical to SEQ ID NO:1.

In embodiments, the Δ12 desaturase comprises amino acids having a sequence set forth as SEQ ID NO:2, or an amino acid sequence which is at least 60%, at least 70%, at least 80%, at least 90% or at least 95% identical to SEQ ID NO:2.

In embodiments, the Δ12 desaturase comprises amino acids having a sequence set forth as SEQ ID NO:3, or an amino acid sequence which is at least 60%, at least 70%, at least 80%, at least 90% or at least 95% identical to SEQ ID NO:3.

In embodiments, the Δ12 desaturase comprises amino acids having a sequence set forth as SEQ ID NO:4, or an amino acid sequence which is at least 60%, at least 70%, at least 80%, at least 90% or at least 95% identical to SEQ ID NO:4.

In embodiments, the Δ6 desaturase comprises amino acids having a sequence set forth as SEQ ID NO:5 or SEQ ID NO:6, or an amino acid sequence which is at least 60%, at least 70%, at least 80%, at least 90% or at least 95% identical to SEQ ID NO:5 or SEQ ID NO:6.

In embodiments, the Δ6 desaturase comprises amino acids having a sequence set forth as SEQ ID NO:5, or an amino acid sequence which is at least 60%, at least 70%, at least 80%, at least 90% or at least 95% identical to SEQ ID NO:5.

In embodiments, the Δ6 desaturase comprises amino acids having a sequence set forth as SEQ ID NO:6, or an amino acid sequence which is at least 60%, at least 70%, at least 80%, at least 90% or at least 95% identical to SEQ ID NO:6.

In embodiments, the Δ9 elongase comprises amino acids having a sequence set forth as any one of SEQ ID NOs: 7 to 12, or an amino acid sequence which is at least 60%, at least 70%, at least 80%, at least 90% or at least 95% identical to any one or more of SEQ ID NOs: 7 to 12.

In embodiments, the Δ9 elongase comprises amino acids having a sequence set forth as SEQ ID NO:7, or an amino acid sequence which is at least 60%, at least 70%, at least 80%, at least 90% or at least 95% identical to SEQ ID NO:7.

In embodiments, the Δ9 elongase comprises amino acids having a sequence set forth as SEQ ID NO:8, or an amino acid sequence which is at least 60%, at least 70%, at least 80%, at least 90% or at least 95% identical to SEQ ID NO:8.

In embodiments, the Δ9 elongase comprises amino acids having a sequence set forth as SEQ ID NO:9, or an amino acid sequence which is at least 60%, at least 70%, at least 80%, at least 90% or at least 95% identical to SEQ ID NO:9.

In embodiments, the Δ9 elongase comprises amino acids having a sequence set forth as SEQ ID NO:10, or an amino acid sequence which is at least 60%, at least 70%, at least 80%, at least 90% or at least 95% identical to SEQ ID NO:10.

In embodiments, the Δ9 elongase comprises amino acids having a sequence set forth as SEQ ID NO:11, or an amino acid sequence which is at least 60%, at least 70%, at least 80%, at least 90% or at least 95% identical to SEQ ID NO:11.

In embodiments, the Δ9 elongase comprises amino acids having a sequence set forth as SEQ ID NO:12, or an amino acid sequence which is at least 60%, at least 70%, at least 80%, at least 90% or at least 95% identical to SEQ ID NO:12.

In embodiments, the Δ6 elongase comprises amino acids having a sequence set forth as SEQ ID NO:13 or an amino acid sequence which is at least 60%, at least 70%, at least 80%, at least 90% or at least 95% identical to SEQ ID NO:13.

In embodiments, the A8 desaturase comprises amino acids having a sequence set forth as SEQ ID NO:14 or an amino acid sequence which is at least 60%, at least 70%, at least 80%, at least 90% or at least 95% identical to SEQ ID NO: 14.

In embodiments, the Δ5 desaturase comprises amino acids having a sequence set forth as SEQ ID NO:15 or SEQ ID NO:16, or an amino acid sequence which is at least 60%, at least 70%, at least 80%, at least 90% or at least 95% identical to SEQ ID NO:15 or SEQ ID NO:16.

In embodiments, the Δ5 desaturase comprises amino acids having a sequence set forth as SEQ ID NO:15, or an amino acid sequence which is at least 60%, at least 70%, at least 80%, at least 90% or at least 95% identical to SEQ ID NO:15.

In embodiments, the Δ5 desaturase comprises amino acids having a sequence set forth as SEQ ID NO:16, or an amino acid sequence which is at least 60%, at least 70%, at least 80%, at least 90% or at least 95% identical to SEQ ID NO:16.

In embodiments, the Δ5 elongase comprises amino acids having a sequence set forth as SEQ ID NO:17 or an amino acid sequence which is at least 60%, at least 70%, at least 80%, at least 90% or at least 95% identical to SEQ ID NO:17.

In embodiments, the Δ4 desaturase comprises amino acids having a sequence set forth as SEQ ID NO:18 or SEQ ID NO:19, or an amino acid sequence which is at least 60%, at least 70%, at least 80%, at least 90% or at least 95% identical to SEQ ID NO:18 or SEQ ID NO: 19.

In embodiments, the Δ4 desaturase comprises amino acids having a sequence set forth as SEQ ID NO:18, or an amino acid sequence which is at least 60%, at least 70%, at least 80%, at least 90% or at least 95% identical to SEQ ID NO:18.

In embodiments, the Δ4 desaturase comprises amino acids having a sequence set forth as SEQ ID NO:19, or an amino acid sequence which is at least 60%, at least 70%, at least 80%, at least 90% or at least 95% identical to SEQ ID NO:19.

In another aspect, the present invention provides a DNA construct, or a combination of DNA constructs, which encodes one or more of the desaturase and elongase enzymes described above, preferably integrated into the genome of a microbial cell. In some embodiments, the DNA construct is a vector.

In another aspect, the present invention provides an isolated strain of Mortierella sp. which comprises a internal transcribed spacer (ITS) region having a nucleotide sequence as shown in any one of SEQ ID NO's 105 to 110, 112, 121, 126 to 146, or a nucleotide sequence at least 90%, at least 95% or at least 99% identical to one or more of SEQ ID NO's 105 to 110, 112, 121, 126 to 146. In an embodiment, the Mortierella sp. is Mortierella alpina which comprises a internal transcribed spacer (ITS) region having a nucleotide sequence as shown in any one of SEQ ID NO's 105 to 110 or 112, or a nucleotide sequence at least 90%, at least 95% or at least 99% identical to one or more of SEQ ID NO's 105 to 110 or 112. In an embodiment, the Mortierella sp. is Mortierella elongata which comprises a internal transcribed spacer (ITS) region having a nucleotide sequence as shown in SEQ ID NO: 121 or SEQ ID NO: 134, or a nucleotide sequence at least 90%, at least 95% or at least 99% identical to one or both of SEQ ID NO: 121 or SEQ ID NO: 134.

In an embodiment, the isolated strain is selected from:
i) yNI0125 deposited under V21/019953 on 12 Oct. 2021 at the National Measurement Institute Australia;
ii) yNI0126 deposited under V21/019951 on 12 Oct. 2021 at the National Measurement Institute Australia;
iii) yNI0127 deposited under V21/019952 on 12 Oct. 2021 at the National Measurement Institute Australia; and
iv) yNI0132 deposited under V21/019954 on 12 Oct. 2021 at the National Measurement Institute Australia.

In another aspect, the present invention provides an isolated strain of Mucor hiemalis which comprises a internal transcribed spacer (ITS) region having a nucleotide sequence as shown in any one of SEQ ID NO's 104, 113 to 120 or 122 to 125, or a nucleotide sequence at least 90%, at least 95% or at least 99% identical to one or more of SEQ ID NO's 104, 113 to 120 or 122 to 125.

In another aspect, the present invention provides an isolated fungal strain which comprises a internal transcribed spacer (ITS) region having a nucleotide sequence as shown in SEQ ID NO: 111, or a nucleotide sequence at least 90% identical, at least 95% or at least 99% to SEQ ID NO: 111.

### Polypeptides

The terms “polypeptide” and “protein” are generally used interchangeably.

A polypeptide or class of polypeptides may be defined by the extent of identity (% identity) of its amino acid sequence to a reference amino acid sequence, or by having a greater % identity to one reference amino acid sequence than to another. The % identity of a polypeptide to a reference amino acid sequence is typically determined by GAP analysis (Needleman and Wunsch, 1970; GCG program) with parameters of a gap creation penalty=5, and a gap extension penalty=0.3. The query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns two sequences over the entire length of the reference amino acid sequence. The polypeptide or class of polypeptides may have the same enzymatic activity as, or a different activity than, or lack the activity of, the reference polypeptide. Preferably, the polypeptide has an enzymatic activity of at least 10%, at least 50%, at least 75% or at least 90%, of the activity of the reference polypeptide.

A polynucleotide defined herein may encode a biologically active fragment of an enzyme such as a desaturase or an elongase. As used herein a “biologically active” fragment is a portion of a polypeptide defined herein which maintains a defined activity of a full-length reference polypeptide, for example possessing desaturase and/or elongase activity or other enzyme activity. Biologically active fragments as used herein exclude the full-length polypeptide. Biologically active fragments can be any size portion as long as they maintain the defined activity. Preferably, the biologically active fragment maintains at least 10%, at least 50%, at least 75% or at least 90%, of the activity of the full-length protein.

With regard to a defined polypeptide or enzyme, it will be appreciated that % identity figures higher than those provided herein will encompass preferred embodiments. Thus, where applicable, in light of the minimum % identity figures, it is preferred that the polypeptide/enzyme comprises an amino acid sequence which is at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO. In an embodiment, for each of the ranges listed above, the % identity does not include 100% i.e. the amino acid sequence is different to the nominated SEQ ID NO.

Amino acid sequence variants/mutants of the polypeptides of the defined herein can be prepared by introducing appropriate nucleotide changes into a nucleic acid defined herein, or by in vitro synthesis of the desired polypeptide. Such variants/mutants include, for example, deletions, insertions or substitutions of residues within the amino acid sequence. A combination of deletion, insertion and substitution can be made to arrive at the final construct, provided that the final peptide product possesses the desired enzyme activity.

Mutant (altered) peptides can be prepared using any technique known in the art. For example, a polynucleotide defined herein can be subjected to in vitro mutagenesis or DNA shuffling techniques as broadly described by Harayama (1998). Products derived from mutated/altered DNA can readily be screened using techniques described herein to determine if they possess, for example, desaturase or elongase activity.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. The sites of greatest interest for substitutional mutagenesis include sites which are not conserved amongst naturally occurring desaturases or elongases. These sites are preferably substituted in a relatively conservative manner in order to maintain enzyme activity. Such conservative substitutions are shown in Table 2 under the heading of “exemplary substitutions”.

In a preferred embodiment a mutant/variant polypeptide has only, or not more than, one or two or three or four conservative amino acid changes when compared to a naturally occurring polypeptide. Details of conservative amino acid changes are provided in Table 2. As the skilled person would be aware, such minor changes can reasonably be predicted not to alter the activity of the polypeptide when expressed in a recombinant cell.

TABLE 2
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

### Polynucleotides

The invention also provides for the use of polynucleotides which may be, for example, a gene, an isolated polynucleotide, or a chimeric genetic construct such as a chimeric DNA. It may be DNA or RNA of genomic or synthetic origin, double-stranded or single-stranded, and combined with carbohydrate, lipids, protein or other materials to perform a particular activity defined herein. The term “polynucleotide” is used interchangeably herein with the term “nucleic acid molecule”.

In an embodiment, the polynucleotide is non-naturally occurring. Examples of non-naturally occurring polynucleotides include, but are not limited to, those that have been codon optimised for expression in microbial cell, those that have been mutated, for example by using methods described herein, and polynucleotides where an open reading frame encoding a protein is operably linked to a promoter to which it is not naturally associated, for example as in the constructs described herein, i.e a promoter that is heterologous with respect to the open reading frame.

As used herein, a “chimeric DNA” or “chimeric genetic construct” or similar refers to any DNA molecule that is not a native DNA molecule in its native location, also referred to herein as a “DNA construct”. Typically, a chimeric DNA or chimeric gene comprises regulatory and transcribed or protein coding sequences that are not found operably linked together in nature i.e. that are heterologous with respect to each other. Accordingly, a chimeric DNA or chimeric gene may comprise regulatory sequences and coding sequences that are derived from different sources, or regulatory sequences and coding sequences derived from the same source, but arranged in a manner different than that found in nature.

An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA technology. The terms “foreign polynucleotide” or “exogenous polynucleotide” or “heterologous polynucleotide” and the like refer to any nucleic acid which is introduced into the genome of a cell by experimental manipulations. Foreign or exogenous genes may be genes that are inserted into a non-native organism, native genes introduced into a new location within the native host, or chimeric genes. A “transgene” is a gene that has been introduced into the genome by a transformation procedure. The terms “genetic modification”, “genetic variation”, “transgenic” and variations thereof include introducing genes into cells by transformation or transduction, mutating genes in cells, deleting genes, and altering or modulating the regulation of a gene by a heritable change in the genome in a cell or organism to which these acts have been done or their progeny. A “genomic region” as used herein refers to a position within the genome where a transgene, or group of transgenes (also referred to herein as a cluster), have been inserted into a cell, or an ancestor thereof. Such regions only comprise nucleotides that have been incorporated by the intervention of a human such as by methods described herein.

The term “exogenous” in the context of a polynucleotide refers to the polynucleotide when present in a cell in an altered amount compared to its native state. In one embodiment, the cell is a cell that does not naturally comprise the polynucleotide. However, the cell may be a cell which comprises a non-endogenous polynucleotide resulting in an altered amount of An exogenous polynucleotide includes production of the encoded polypeptide. polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically such chimeric polynucleotides comprise at least an open reading frame encoding a polypeptide operably linked to a promoter suitable of driving transcription of the open reading frame in a cell of interest.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 35%, more preferably at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO. In an embodiment, for each of the ranges listed above, the % identity does not include 100% i.e. the nucleotide sequence is different to the nominated SEQ ID NO.

Polynucleotides may possess, when compared to naturally occurring molecules, one or more mutations which are deletions, insertions, or substitutions of nucleotide residues. Polynucleotides which have mutations relative to a reference sequence can be either naturally occurring (that is to say, isolated from a natural source) or synthetic (for example, by performing site-directed mutagenesis or DNA shuffling on the nucleic acid as described above). It is thus apparent that polynucleotides can be either from a naturally occurring source or recombinant. Preferred polynucleotides are those which have coding regions that are codon-optimised for translation in microbial cells, as is known in the art.

### Recombinant Vectors

Recombinant expression can be used to produce recombinant microbes of the invention. Recombinant vectors contain heterologous polynucleotide sequences, that is, polynucleotide sequences that are not naturally found adjacent to polynucleotide molecules defined herein that preferably are derived from a species other than the species from which the polynucleotide molecule(s) are derived. The vector can be either RNA or DNA and typically is a plasmid. Plasmid vectors typically include additional nucleic acid sequences that provide for easy selection, amplification, and transformation of the expression cassette in prokaryotic cells, e.g., pYES-derived vectors, pUC-derived vectors, pSK-derived vectors, pGEM-derived vectors, pSP-derived vectors, or pBS-derived vectors. Suitable yeast expression vectors include the pPIC series of vectors, yeast integrating plasmids (YIp), yeast replicating plasmids (YRp), yeast centromere plasmids (YCp), and yeast episomal plasmids (YEp). Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of microbial cells. The recombinant vector may comprise more than one polynucleotide defined herein, for example three, four, five or six polynucleotides defined herein in combination, preferably a chimeric genetic construct described herein, each polynucleotide being operably linked to expression control sequences that are operable in the cell.

“Operably linked” as used herein refers to a functional relationship between two or more nucleic acid (e.g., DNA) segments. Typically, it refers to the functional relationship of transcriptional regulatory element (promoter) to a transcribed sequence. For example, a promoter is operably linked to a coding sequence, such as a polynucleotide defined herein, if it stimulates or modulates the transcription of the coding sequence in an appropriate cell. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance. For example, an intron in a 5′ UTR sequence or towards the 5′ end of a protein coding region can contain a transcriptional enhancer, providing an increased expression level, for example an FBAIN promoter region.

To facilitate identification of transformants, the nucleic acid construct desirably comprises a selectable or screenable marker gene as, or in addition to, the foreign or exogenous polynucleotide. By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., β-glucuronidase, luciferase, GFP or other enzyme activity not present in untransformed cells). The marker gene and the nucleotide sequence of interest do not have to be linked. The actual choice of a marker is not crucial as long as it is functional (i.e., selective) in combination with the cells of choice.

Examples of selectable markers are markers that confer antibiotic resistance such as hygromycin, nourseothricin, ampicillin, erythromycin, chloramphenicol or tetracycline resistance, preferably hygromycin or kanamycin resistance.

Recombinant yeast of the invention may comprise a reporter gene which either encodes a galactosidase or a selectable growth marker.

The “galactosidase” may be any enzyme which cleaves a terminal galactose residue(s) from a variety of substrates, and which is able to also cleave a substrate to produce a detectable signal. In an embodiment, the galactosidase is a β-galactosidase such as bacterial (for instance from E. coli) LacZ. In an alternate embodiment, the galactosidase is an a-galactosidase such as yeast (for instance S. cerevisiae) Mel-1. β-galactosidase activity may be detected using substrates for the enzyme such as X-gal (5-bromo-4-chloro-indolyl-β-D-galactopyranoside) which forms an intense blue product after cleavage, ONPG (o-nitrophenyl galactoside) which forms a water soluble yellow dye with an absorbance maximum at about 420 nm after cleavage, and CPRG (chlorophenol red-β-D-galactopyranoside) which yields a water-soluble red product measurable by spectrophotometry after cleavage. α-galactosidase activity may be detected using substrates for the enzyme such as o-nitrophenyl α-D-galactopyranoside which forms an indigo dye after cleavage, and chlorophenol red-α-D-galactopyranoside which yields a water-soluble red product measurable by spectrophotometry after cleavage. Kits for detecting galactosidase expression in yeast are commercially available, for instance the β-galactosidase (LacZ) expression kit from Thermo Scientific.

Preferably, the selectable growth marker is a nutritional marker or antibiotic resistance marker.

Typical yeast selectable nutritional markers include, but are not limited to, LEU2, TRP1, HIS3, HIS4, URA3, URA5, SFAI, ADE2, MET15, LYS5, LYS2, ILV2, FBA1, PSE1, PDI1 and PGK1. Those skilled in the art will appreciate that any gene whose chromosomal deletion or inactivation results in an unviable host, so called essential genes, can be used as a selective marker if a functional gene is provided on the, for example, plasmid, as demonstrated for PGK1 in a pgk1 yeast strain. Suitable essential genes can be found within the Stanford Genome Database (SGD) (http:://db.yeastgenome.org). Any essential gene product (e.g. PDI1, PSE1, PGK1 or FBA1) which, when deleted or inactivated, does not result in an auxotrophic (biosynthetic) requirement, can be used as a selectable marker on a, for example, plasmid in a yeast host cell that, in the absence of the plasmid, is unable to produce that gene product, to achieve increased plasmid stability without the disadvantage of requiring the cell to be cultured under specific selective conditions. By “auxotrophic (biosynthetic) requirement” we include a deficiency which can be complemented by additions or modifications to the growth medium.

### Expression

Expression vectors can direct gene expression in microbial cells. As used herein, an expression vector is a vector that is capable of transforming a host cell and of effecting expression of one or more specified polynucleotide molecule(s). Expression vectors useful for the invention contain regulatory sequences such as transcription control sequences, translation control sequences, origins of replication, and other regulatory sequences that are compatible with the recombinant cell and that control the expression of polynucleotide molecules of the present invention. In particular, polynucleotides or vectors useful for the present invention include transcription control sequences. Transcription control sequences are sequences which control the initiation, elongation, and termination of transcription. Particularly important transcription control sequences are those which control transcription initiation, such as promoter and enhancer sequences. Suitable transcription control sequences include any transcription control sequence that can function in at least one of the recombinant cells of the present invention. The choice of the regulatory sequences used depends on the target microbial cell. A variety of such transcription control sequences are known to those skilled in the art.

Yeast cells are typically transformed by chemical methods (e.g., as described by Rose et al., 1990, Methods in Yeast Genetics, Cold Spring Harbor Laboratory Press, Cold Spring Harbor, N.Y., and in Kawai et al., 2010). The cells are typically treated with lithium acetate to achieve transformation efficiencies of approximately 104 colony-forming units (transformed cells)/μg of DNA. Other standard procedures for transforming yeast include i) the spheroplast method which, as the name suggests, relies on the production of yeast spheroplasts, ii) the biolistic method where DNA coated metal microprojectiles are shot into the cells, and iii) the glass bead methods which relies on the agitation of the yeast cells with glass beads and the DNA to be delivered to the cell. Of course, any suitable means of introducing nucleic acids into yeast cells can be used.

It is well known that transformation of organisms, such as yeast, with exogenous plasmids can lead to clonal differences in the penetrance of the transformed gene, due to differences in copy number or other factors. It is therefore advisable to screen two or more independent clonal isolates for each transformed receptor in order to maximise the likelihood of identifying suitable receptor=ligand pairs during screening. Different clonal isolates may be screened independently or may be combined into a single well for screening. The latter option may be particularly convenient where a nutritional reporter is used rather than a colorimetric reporter.

“Constitutive promoter” refers to a promoter that directs expression of an operably linked transcribed sequence in the cell without the need to be induced by specific growth conditions. Examples of constitutive promoters useful for yeast cells of the invention include, but are not limited to, a yeast PGK (phosphoglycerate kinase) promoter, a yeast ADH-1 (alcohol dehydrogenase) promoter, a yeast ENO (enolase) promoter, a yeast glyceraldehyde 3-phosphate dehydrogenase promoter (GPD) promoter, a yeast PYK-1 (pyruvate kinase) promoter, a yeast translation-elongation factor-1-alpha promoter (TEF) promoter and a yeast CYC-1 (cytochrome c-oxidase promoter) promoter. In a preferred embodiment, a yeast promoter is a S. cerevisiae promoter. In another embodiment, the constitutive promoter may not have been derived from yeast. Examples of such promoters useful for the invention include, but are not limited to, the cauliflower mosaic virus 35S promoter, the glucocorticoid response element, and the androgen response element. The constitutive promoter may be the naturally occurring molecule or a variant thereof comprising, for example, one, two or three nucleotide substitutions which do not abolish (and preferably enhance) promoter function.

Recombinant DNA technologies can be used to improve expression of a transformed polynucleotide molecule by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotide molecules defined herein include, but are not limited to, integration of the polynucleotide molecule into one or more host cell chromosomes, addition of stability sequences to mRNAs, substitutions or modifications of transcription control signals (e.g., promoters, operators, enhancers), substitutions or modifications of translational control signals (e.g., ribosome binding sites, Shine-Dalgarno sequences), modification of polynucleotide molecules to correspond to the codon usage of the host cell, and the deletion of sequences that destabilize transcripts.

### Other Genetic Modification Techniques

Any method can be used to introduce a nucleic acid molecule into a microbial cell and many such methods are well known. For example, transformation and electroporation are common methods for introducing nucleic acid into yeast cells (see, e.g., Gietz et al., 1992; Ito et al., 1983; and Becker et al., 1991).

In an embodiment, the integration of a gene of interest into a specific chromosomal site in a microbial cell occurs via homologous recombination. According to this embodiment, an integration cassette containing a module comprising at least one marker gene and/or the gene to be integrated (internal module) is flanked on either side by DNA fragments homologous to those of the ends of the targeted integration site (recombinogenic sequences). After transforming the microbial cell with the cassette by appropriate methods, a homologous recombination between the recombinogenic sequences may result in the internal module replacing the chromosomal region in between the two sites of the genome corresponding to the recombinogenic sequences of the integration cassette (Orr-Weaver et al., 1981).

In an embodiment, the integration cassette for integration of a gene of interest into a microbial cell includes the heterologous gene under the control of an appropriate promoter together with a selectable marker flanked by recombinogenic sequences for integration of a heterologous gene into the microbial cell chromosome. In an embodiment, the heterologous gene includes any of the fatty acid biosynthesis genes described herein.

Where deletion of an endogenous gene is desired, the integration cassette can comprise a selectable marker (without any other heterologous gene sequence) flanked by DNA fragments homologous to those of the ends (and/or neighbouring sequences) of the endogenous gene targeted for deletion. Other methods suitable for deleting or mutating endogenous genes (e.g., using site-specific or RNA-guided nucleases) are described below.

The selectable marker gene can be any marker gene used in microbial cells, including but not limited to, HIS3, TRP1, LEU2, URA3, bar, ble, hph, and kan. The recombinogenic sequences can be chosen at will, depending on the desired integration site suitable for the desired application.

In another embodiment, integration of a gene into the chromosome of the microbial cell may occur via random integration (Kooistra et al., 2004).

Additionally, in an embodiment, certain introduced marker genes are removed from the genome using techniques well known to those skilled in the art. For example, URA3 marker loss can be obtained by plating URA3 containing cells in FOA (5-fluoro-orotic acid) containing medium and selecting for FOA resistant colonies (Boeke et al., 1984).

The exogenous nucleic acid molecule contained within a microbial cell of the disclosure can be maintained within that cell in any form. For example, exogenous nucleic acid molecules can be integrated into the genome of the cell or maintained in an episomal state that can stably be passed on (“inherited”) to daughter cells. Such extra-chromosomal genetic elements (such as plasmids, mitochondrial genome, etc.) can additionally contain selection markers that ensure the presence of such genetic elements in daughter cells. Moreover, the microbial cells can be stably or transiently transformed. In addition, the microbial cells described herein can contain a single copy, or multiple copies of a particular exogenous nucleic acid molecule as described above.

### Genome Editing Using Site-Specific Nucleases

Genome editing uses engineered nucleases composed of sequence specific DNA binding domains fused to a non-specific DNA cleavage module. These chimeric nucleases enable efficient and precise genetic modifications (including deletions, mutations and insertions) by inducing targeted DNA double stranded breaks that stimulate the cell's endogenous cellular DNA repair mechanisms to repair the induced break. Such mechanisms include, for example, error prone non-homologous end joining (NHEJ) and homology directed repair (HDR).

In the presence of donor plasmid with extended homology arms, HDR can lead to the introduction of single or multiple transgenes to correct or replace existing genes. In the absence of donor plasmid, NHEJ-mediated repair yields small insertion or deletion mutations of the target that cause gene disruption.

Engineered nucleases useful in the methods of the present invention include zinc finger nucleases (ZFNs) and transcription activator-like (TAL) effector nucleases (TALEN).

Typically nuclease encoded genes are delivered into cells by plasmid DNA, viral vectors or in vitro transcribed mRNA. The use of fluorescent surrogate reporter vectors also allows for enrichment of ZFN- and TALEN-modified cells. As an alternative to ZFN gene-delivery systems, cells can be contacted with purified ZFN proteins which are capable of crossing cell membranes and inducing endogenous gene disruption.

A zinc finger nuclease (ZFN) comprises a DNA-binding domain and a DNA-cleavage domain, wherein the DNA binding domain is comprised of at least one zinc finger and is operatively linked to a DNA-cleavage domain. The zinc finger DNA-binding domain is at the N-terminus of the protein and the DNA-cleavage domain is located at the C-terminus of said protein.

A ZFN must have at least one zinc finger. In a preferred embodiment, a ZFN would have at least three zinc fingers in order to have sufficient specificity to be useful for targeted genetic recombination in a host cell. Typically, a ZFN having more than three zinc fingers would have progressively greater specificity with each additional zinc finger.

The zinc finger domain can be derived from any class or type of zinc finger. In a particular embodiment, the zinc finger domain comprises the Cis₂His₂ type of zinc finger that is very generally represented, for example, by the zinc finger transcription factors TFIIIA or Sp1. In a preferred embodiment, the zinc finger domain comprises three Cis₂His₂ type zinc fingers. The DNA recognition and/or the binding specificity of a ZFN can be altered in order to accomplish targeted genetic recombination at any chosen site in cellular DNA. Such modification can be accomplished using known molecular biology and/or chemical synthesis techniques (see, for example, Bibikova et al., 2002).

The ZFN DNA-cleavage domain is derived from a class of non-specific DNA cleavage domains, for example the DNA-cleavage domain of a Type II restriction enzyme such as FokI (Kim et al., 1996). Other useful endonucleases may include, for example, HhaI, HindIII, Nod, BbvCI, EcoRI, BglI, and AlwI.

In order to target genetic recombination or mutation according to a preferred embodiment of the present invention, two 9 bp zinc finger DNA recognition sequences must be identified in the host microbial cell DNA. These recognition sites will be in an inverted orientation with respect to one another and separated by about 6 bp of DNA. ZFNs are then generated by designing and producing zinc finger combinations that bind DNA specifically at the target locus, and then linking the zinc fingers to a DNA cleavage domain.

ZFN activity can be improved through the use of transient hypothermic culture conditions to increase nuclease expression levels (Doyon et al., 2010) and co-delivery of site-specific nucleases with DNA end-processing enzymes (Certo et al., 2012). The specificity of ZFN-mediated genome editing can be improved by use of zinc finger nickases (ZFNickases) which stimulate HDR without activation the error-prone NHE-J repair pathway (Kim et al., 2012; Wang et al., 2012; Ramirez et al., 2012; McConnell Smith et al., 2009).

A transcription activator-like (TAL) effector nuclease (TALEN) comprises a TAL effector DNA binding domain and an endonuclease domain.

TAL effectors are proteins of plant pathogenic bacteria that are injected by the pathogen into the plant cell, where they travel to the nucleus and function as transcription factors to turn on specific plant genes. The primary amino acid sequence of a TAL effector dictates the nucleotide sequence to which it binds. Thus, target sites can be predicted for TAL effectors, and TAL effectors can be engineered and generated for the purpose of binding to particular nucleotide sequences.

Fused to the TAL effector-encoding nucleic acid sequences are sequences encoding a nuclease or a portion of a nuclease, typically a nonspecific cleavage domain from a type II restriction endonuclease such as FokI (Kim et al., 1996). Other useful endonucleases may include, for example, HhaI, HindIII, Nod, BbvCI, EcoRI, BglI, and AlwI. The fact that some endonucleases (e.g., FokI) only function as dimers can be capitalized upon to enhance the target specificity of the TAL effector. For example, in some cases each FokI monomer can be fused to a TAL effector sequence that recognizes a different DNA target sequence, and only when the two recognition sites are in close proximity do the inactive monomers come together to create a functional enzyme. By requiring DNA binding to activate the nuclease, a highly site-specific restriction enzyme can be created.

A sequence-specific TALEN can recognize a particular sequence within a preselected target nucleotide sequence present in a cell. Thus, in some embodiments, a target nucleotide sequence can be scanned for nuclease recognition sites, and a particular nuclease can be selected based on the target sequence. In other cases, a TALEN can be engineered to target a particular cellular sequence.

### Genome Editing Using Programmable RNA-Guided DNA Endonucleases

Distinct from the site-specific nucleases described above, the clustered regulatory interspaced short palindromic repeats (CRISPR)/Cas system provides an alternative to ZFNs and TALENs for inducing targeted genetic alterations. In bacteria, the CRISPR system provides acquired immunity against invading foreign DNA via RNA-guided DNA cleavage.

CRISPR systems rely on CRISPR RNA (crRNA) and transactivating chimeric RNA (tracrRNA) for sequence-specific silencing of invading foreign DNA. Three types of CRISPR/Cas systems exist: in type II systems, Cas9 serves as an RNA-guided DNA endonuclease that cleaves DNA upon crRNA-tracrRNA target recognition. CRISPR RNA base pairs with tracrRNA to form a two-RNA structure that guides the Cas9 endonuclease to complementary DNA sites for cleavage.

CRISPR loci are a distinct class of interspersed short sequence repeats (SSRs) that were first recognized in E. coli (Ishino et al., 1987; Nakata et al., 1989). Similar interspersed SSRs have, been identified in Haloferax mediterranei, Streptococcus pyogenes, Anabaena, and Mycobacterium tuberculosis (Groenen et al., 1993; Hoe et al., 1999; Masepohl et al., 1996; Mojica et al., 1995).

The CRISPR loci differ from other SSRs by the structure of the repeats, which have been termed short regularly spaced repeats (SRSRs) (Janssen et al., 2002; Mojica et al., 2000). The repeats are short elements that occur in clusters, that are always regularly spaced by unique intervening sequences with a constant length (Mojica et al., 2000). Although the repeat sequences are highly conserved between strains, the number of interspersed repeats and the sequences of the spacer regions differ from strain to strain (van Embden et al., 2000).

The common structural characteristics of CRISPR loci are described in Jansen et al. (2002) as (i) the presence of multiple short direct repeats, which show no or very little sequence variation within a given locus; (ii) the presence of non-repetitive spacer sequences between the repeats of similar size; (iii) the presence of a common leader sequence of a few hundred basepairs in most species harbouring multiple CRISPR loci; (iv) the absence of long open reading frames within the locus; and (v) the presence of one or more cas genes.

CRISPRs are typically short partially palindromic sequences of 24-40 bp containing inner and terminal inverted repeats of up to 11 bp. Although isolated elements have been detected, they are generally arranged in clusters (up to about 20 or more per genome) of repeated units spaced by unique intervening 20-58 bp sequences. CRISPRs are generally homogenous within a given genome with most of them being identical. However, there are examples of heterogeneity in, for example, the Archaea (Mojica et al., 2000).

As used herein, the term “cas gene” refers to one or more cas genes that are generally coupled associated or close to or in the vicinity of flanking CRISPR loci. A comprehensive review of the Cas protein family is presented in Haft et al. (2005). The number of cas genes at a given CRISPR locus can vary between species.

### Cell Culture

Effective culture conditions are known to those skilled in the art and include, but are not limited to, suitable media, bioreactor, temperature, pH and oxygen conditions that permit lipid production. A suitable medium refers to any medium in which a cell is cultured to produce lipid defined herein. Such medium typically comprises an aqueous medium having assimilable carbon, nitrogen and phosphate sources, and appropriate salts, minerals, metals and other nutrients, such as vitamins. Cells defined herein can be cultured in conventional fermentation bioreactors, shake flasks, test tubes, microtiter dishes, and petri plates. Culturing can be carried out at a temperature, pH and oxygen content appropriate for a recombinant cell. Such culturing conditions are within the expertise of one of ordinary skill in the art.

### Lipid Extraction

Extraction of the lipid from microbial cell of the invention uses analogous methods to those known in the art for lipid extraction from oleaginous microorganisms, such as for example described in Patel et al. (2018). In one embodiment, the extraction is performed by solvent extraction where an organic solvent (e.g., hexane or a mixture of hexane and ethanol) is mixed with at least the biomass, preferably after the biomass is dried and ground, but it can also be performed under wet conditions. The solvent dissolves the lipid in the cells, which solution is then separated from the biomass by a physical action (e.g., ultrasonication). Ultrasonication is one of the most extensively used pretreatment methods to disrupt the cellular integrity of microbial cells. Other pretreatment methods can include microwave irradiation, high-speed homogenization, high-pressure homogenization, bead beating, autoclaving, and thermolysis. The organic solvent can then be separated from the non-polar lipid (e.g., by distillation). This second separation step yields non-polar lipid from the cells and can yield a re-usable solvent if one employs conventional vapor recovery.

In solvent extraction, an organic solvent (e.g., hexane or a mixture of hexane and ethanol) is mixed with at least the biomass of the microbial cell, preferably after the biomass is dried and ground. The solvent dissolves the lipid in the biomass and the like, which solution is then separated from the biomass by mechanical action (e.g., with the processes above). This separation step can also be performed by filtration (e.g., with a filter press or similar device) or centrifugation etc. The organic solvent can then be separated from the non-polar lipid (e.g., by distillation). This second separation step yields non-polar lipid from the microbial cell and can yield a re-usable solvent if one employs conventional vapor recovery.

The lipid extracted from the microbial cells of the invention may be subjected to normal oil processing procedures. As used herein, the term “purified” when used in connection with lipid of the invention typically means that that the extracted lipid has been subjected to one or more processing steps of increase the purity of the lipid component. For example, a purification step may comprise one or more or all of the group consisting of: degumming, deodorising, decolourising, drying and/or fractionating the extracted oil. However, as used herein, the term “purified” does not include a transesterification process or other process which alters the fatty acid composition of the lipid or oil of the invention so as to change the fatty acid composition of the total fatty acid content. Expressed in other words, in a preferred embodiment the fatty acid composition of the purified lipid is essentially the same as that of the unpurified lipid.

### Degumming

Degumming is an early step in the refining of lipids in a liquid form (oil) and its primary purpose is the separation of most of the phospholipids from the oil, which may be present as approximately 1-2% of the total extracted lipid. Addition of ˜2% of water, typically containing phosphoric acid, at 70-80° C. to the crude oil results in the separation of most of the phospholipids accompanied by trace metals and pigments. The insoluble material that is removed is mainly a mixture of phospholipids and is also known as lecithin. Degumming can be performed by addition of concentrated phosphoric acid to the crude extracted lipid to convert non-hydratable phosphatides to a hydratable form, and to chelate minor metals that are present. Gum is separated from the oil by centrifugation. The recovered gum comprising ω6 fatty acids, other than LA alone, is encompassed in the present invention.

### Alkali Refining

Alkali refining is one of the refining processes for treating lipid in the form of an oil, sometimes also referred to as neutralization. It usually follows degumming and precedes bleaching. Following degumming, the oil can treated by the addition of a sufficient amount of an alkali solution to titrate all of the fatty acids and phosphoric acids, and removing the soaps thus formed. Suitable alkaline materials include sodium hydroxide, potassium hydroxide, sodium carbonate, lithium hydroxide, calcium hydroxide, calcium carbonate and ammonium hydroxide. This process is typically carried out at room temperature and removes the free fatty acid fraction. Soap is removed by centrifugation or by extraction into a solvent for the soap, and the neutralised oil is washed with water. If required, any excess alkali in the oil may be neutralized with a suitable acid such as hydrochloric acid or sulphuric acid.

### Bleaching

Bleaching is a refining process in which oils are heated at 90-120° C. for 10-30 minutes in the presence of a bleaching earth (0.2-2.0%) and in the absence of oxygen by operating with nitrogen or steam or in a vacuum. This step in oil processing is designed to remove unwanted pigments and the process also removes oxidation products, trace metals, sulphur compounds and traces of soap.

### Deodorization

Deodorization is a treatment of oils and fats at a high temperature (200-260° C.) and low pressure (0.1-1 mm Hg). This is typically achieved by introducing steam into the oil at a rate of about 0.1 ml/minute/100 ml of oil. After about 30 minutes of sparging, the oil is allowed to cool under vacuum. The oil is typically transferred to a glass container and flushed with argon before being stored under refrigeration. This treatment improves the colour of the oil and removes a majority of the volatile substances or odorous compounds including any remaining free fatty acids, monoacylglycerols and oxidation products.

### Transesterification

As used herein, “transesterification” means a process that exchanges the fatty acids within and between TAGs (interesterification) or transfers the fatty acids to another alcohol to form an ester. This may initially involve releasing fatty acids from the TAGs as free fatty acids or it may directly produce fatty acid esters, preferably fatty acid methyl esters or ethyl esters. In a transesterification reaction of the TAG with an alcohol such as methanol or ethanol, the alkyl group of the alcohol forms an ester linkage with the acyl groups (including the SCFA) of the TAG.

### Food, Feedstuffs, Beverages and Compositions

The present invention includes compositions which can be used as a food or beverage ingredient, a food or beverage for human consumption or a feedstuff for animal consumption, preferably at least a food for human consumption. The compositions can also be added to a food, beverage or feedstuff to increase the “meatiness” of the aroma and/or flavour of the food, beverage or feedstuff (e.g., to increase the amount of volatile compounds produced that are known to have a meat-associated aroma). For purposes of the present invention, a food, beverage or feedstuff is a preparation for human or animal consumption which when taken into the body (a) serve to nourish or build up tissues or supply energy; and/or (b) maintain, restore or support adequate nutritional status or metabolic function. A food or beverage ingredient is a composition that is capable of being used as a component of a food or beverage together with at least one other ingredient other than water, such as, for example, macronutrients, protein, carbohydrate, vitamins, and/or minerals.

Suitable foods/feedstuffs include meat substitutes, soup bases, stew bases, snack foods, bouillon powders, bouillon cubes, flavour packets, or frozen food products. Meat substitutes can be formulated, for example, as hot dogs, burgers, ground meat, sausages, steaks, filets, roasts, breasts, thighs, wings, meatballs, meatloaf, bacon, strips, fingers, nuggets, cutlets, or cubes. Ingredients and methods for producing food, feedstuffs and beverages, including meat substitutes, are well known in the art (see e.g., WO2008124370, WO2013010042, WO2015153666 and WO2017070303) and can be employed with the extracted microbial lipids, microbial cells and/or compositions of the present invention to produce a food, feedstuffs and beverages of the present invention that comprises the extracted microbial lipids, microbial cells and/or compositions.

A food, beverage or feedstuff of the invention comprises, for example, extracted lipid of the invention, the microbial cell of the invention, or both extracted lipid and microbial cells of the invention, the microbial cell extract or the composition of the invention. In some examples, the extracted lipid and/or microbial cell have been heated prior to incorporation into the food, such as in the presence of a sugar and an amino acid or derivative, under conditions suitable to produce one or more (e.g. at least or about 2, 3, 4, 5, 6, 7, 8, 9, 10, 11, 12, 13, 14, 15, 16, 17, 18, 19, 20, 21, 22, 23, 24, 25, 26, 26, 28, 29, 30 or 31) volatile compounds indicative of meat-like or meat-associated aromas and flavours, for example volatile compounds such as 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, and 1,3,5-thitriane. In some examples, one or more (e.g. 2, 3, 4, 5, 6, 7, 8 or 9) volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol are produced. In other embodiments, one or more (e.g. 2, 3, 4 or 5) volatile compound(s) selected from 1-pentanal, 3-octanone, 2-octen-1-ol, 1-nonanol and 1-octanol, and optionally 1,3-bis(1,1-dimethylethyl)-benzene are produced. As would be appreciated, the amounts and ratios of various fatty acids (and in particular the ω6 fatty acids (e.g. ARA, GLA, DGLA, EDA, DTA and/or DPA-ω6) in the extracted microbial lipid will change when one or more of these volatile compounds are produced from the reaction between the fatty acids on the polar lipids, the sugar and the amino acid. Consequently, the lipid remaining after the reaction can have a different fatty acid profile compared to the “starting” extracted microbial lipid. Thus, in some examples, a food, beverage or feedstuff of the invention comprises lipids wherein the lipids are a product of a reaction between an extracted microbial lipid of the invention, an amino acid or derivative, and a sugar under conditions suitable to produce at least two compounds which have a meat-associated flavour and/or aroma. In particular examples, the conditions include heating, such as at a temperature of at least about 100° C., 110° C., 120° C., 130° or 140° C., over a period of time (e.g. as described further below) and with sufficient quantities or concentrations of the sugar and amino acid or derivative to produce the volatile compounds.

The food may either be in a solid or liquid form, for example in the form of a powder, solution, suspension, slurry or emulsion. Additionally, the composition may include edible macronutrients, protein, carbohydrate, vitamins, and/or minerals in amounts desired for a particular use. The amounts of these ingredients will vary depending on whether the composition is intended for use with normal individuals or for use with individuals having specialized needs, such as individuals suffering from metabolic disorders and the like.

Examples of suitable ingredients with nutritional value include, but are not limited to, macronutrients such as edible fats, carbohydrates and proteins. Examples of such edible fats other than the lipids of the invention include, but are not limited to, palm oil, canola oil, soybean oil, corn oil, sunflower seed oil, safflower seed oil, cottonseed oil, coconut oil, borage oil, fungal oil, black current oil, and mono- and diglycerides. Examples of such carbohydrates include (but are not limited to): glucose, a mixture of glucose and fructose, edible lactose, and hydrolyzed starch. Additionally, examples of proteins which may be utilized in the nutritional composition of the invention include (but are not limited to) soy proteins, mycoproteins (e.g Rhiza mycoproteins), seitan, pea protein, potato protein, electrodialysed whey, electrodialysed skim milk, milk whey, or the hydrolysates of these proteins. In some examples, the protein is a textured or structured protein product, which comprises protein fiber networks and/or aligned protein fibers that produce meat-like textures. It can be obtained from a dough after application of mechanical energy (e.g., extrusion, spinning, agitating, shaking, shearing, pressure, turbulence, impingement, confluence, beating, friction, wave), radiation energy (e.g., microwave, electromagnetic), thermal energy (e.g., heating, steam texturizing), enzymatic activity (e.g., transglutaminase activity), chemical reagents (e.g., pH adjusting agents, kosmotropic salts, chaotropic salts, gypsum, surfactants, emulsifiers, fatty acids, amino acids), other methods that lead to protein denaturation and protein fiber alignment, or combinations of these methods, followed by fixation of the fibrous and/or aligned structure (e.g., by rapid temperature and/or pressure change, rapid dehydration, chemical fixation, redox), and optional post-processing after the fibrous and/or aligned structure is generated and fixed (e.g., hydrating, marinating, drying, coloring).

With respect to vitamins and minerals, the following may be added to the food, beverage or feedstuff of the present invention: calcium, phosphorus, potassium, sodium, chloride, magnesium, manganese, iron, copper, zinc, selenium, iodine, and Vitamins A, E, D, C, and the B complex. The iron may be provided in the form of iron bound to heme, or a form other than iron bound to heme, preferably in the form of a ferrous salt. Other such vitamins and minerals may also be added.

Additional ingredients include food-grade oils such as canola, corn, sunflower, soybean, olive or coconut oil, seasoning agents such as edible salts (e.g., sodium or potassium chloride) or herbs (e.g., rosemary, thyme, basil, sage, or mint), flavouring agents, proteins (e.g., soy protein isolate, wheat glutin, pea vicilin, and/or pea legumin), protein concentrates (e.g., soy protein concentrate), emulsifiers (e.g., lecithin), gelling agents (e.g., k-carrageenan or gelatin), fibers (e.g., bamboo filer or inulin), or minerals (e.g., iodine, zinc, and/or calcium).

Foods and feedstuffs described herein also can include a natural coloring agent such as turmeric or beet juice, or an artificial coloring agent such as azo dyes, triphenylmethanes, xanthenes, quinines, indigoids, titanium dioxide, red #3, red #40, blue #1, or yellow #5.

Foods and feedstuffs described herein also can include meat shelflife extenders such as carbon monoxide, nitrites, sodium metabisulfite, Bombal, vitamin E, rosemary extract, green tea extract, catechins and other anti-oxidants.

The components utilized in the food, beverage or feedstuff of the present invention can be of semi-purified or purified origin. By semi-purified or purified is meant a material which has been prepared by purification of a natural material or by de novo synthesis.

In an embodiment, the food, beverage or feedstuff has no components derived from an animal. Thus, in a preferred embodiment, at least some of the ingredients are plant material or material derived from a plant. In some embodiments, the food, beverage or feedstuff can be soy-free, wheat-free, yeast-free, MSG-free, and/or free of protein hydrolysis products, and can taste meaty, highly savory, and without off odors or flavours or reduced levels thereof.

In addition, the microbial lipids, microbial cells and/or compositions of the invention can be used to modulate the taste and/or aroma profile of other food products (e.g., meat replicas, meat substitutes, tofu, mock duck or a gluten-based vegetable product, textured vegetable protein such as textured soy protein, pork, fish, lamb, or poultry products such as chicken or turkey products) and can be applied to the other food product before or during cooking. In some embodiments, using the microbial lipids, microbial cells and/or compositions described herein can provide a particular meaty taste and smell, for example, the taste and smell of beef, to a non-meat product or to a poultry product.

In some embodiments, the compositions, foods, feedstuffs and beverages described herein comprise components required for causing a Maillard reaction upon heating the composition. For example, the composition may comprise one or both of (i) a sugar, sugar alcohol, sugar acid, or sugar derivative, and (ii) and an amino acid or derivative thereof.

Suitable sugars, sugar alcohols, sugar acids, and sugar derivatives include glucose, fructose, ribose, sucrose, arabinose, glucose-6-phosphate, fructose-6-phosphate, fructose 1,6-diphosphate, inositol, maltose, molasses, maltodextrin, glycogen, galactose, lactose, ribitol, gluconic acid and glucuronic acid, amylose, amylopectin, and xylose and combinations thereof.

Suitable amino acids and derivatives thereof include cysteine, cystine, a cysteine sulfoxide, allicin, selenocysteine, methionine, isoleucine, leucine, lysine, phenylalanine, threonine, tryptophan, 5-hydroxytryptophan, valine, arginine, histidine, alanine, asparagine, aspartate, glutamate, glutamine, glycine, proline, serine, and tyrosine.

The composition, foods, feedstuffs and beverages may also comprise another one or more other flavour precursors including oils (e.g., vegetable oils), free fatty acids, alpha-hydroxy acids, dicarboxylic acids, nucleosides, nucleotides, vitamins, peptides, protein hydrolysates, extracts, phospholipids, lecithin, and organic molecules.

Foods, feedstuffs, beverages and compositions described herein can be packaged in various ways, including being sealed within individual packets or shakers, such that the composition can be sprinkled or spread on top of a food product before or during cooking.

Foods, beverages and feedstuffs described herein can be assessed for flavour and aroma using trained human panelists. The evaluations can involve eyeing, feeling, chewing, smelling and tasting of the product to judge product appearance, color, integrity, texture, flavour, and mouth feel, etc, preferably at least smelling the food, beverage or feedstuff. Panelists can be served samples under red or under white light. A scale can be used to rate the overall acceptability or quality of the food or specific quality attributes such meatiness, texture, and flavour. The foods, feedstuffs and beverages can also be presented to animals such as pet animals to assess their attractiveness to those animals.

In some embodiments, a food, beverage or feedstuff described herein can be compared to another product (e.g., meat or meat substitute) based upon olfactometer readings. In various embodiments, the olfactometer can be used to assess odor concentration and odor thresholds, odor suprathresholds with comparison to a reference gas, hedonic scale scores to determine the degree of appreciation, or relative intensity of odors.

In some embodiments, volatile chemicals identified using GCMS can be evaluated. For example, a human can rate the experience of smelling the chemical responsible for a certain peak. This information could be used to further refine the profile of flavour and aroma compounds produced by the compositions of the present invention.

Characteristic flavour and fragrance components are mostly produced during the cooking process by chemical reactions molecules including amino acids, fats and sugars which are found in plants as well as meat. Therefore, in some embodiments, a food, beverage or feedstuff is tested for similarity to meat during or after cooking. In some embodiments human ratings, human evaluation, olfactometer readings, or GC-MS measurements, or combinations thereof, are used to create an olfactory map of the food or feedstuff. Similarly, an olfactory map of the food, beverage or feedstuff, for example, a meat replica, can be created. These maps can be compared to assess how similar the cooked food or feedstuff is to meat.

The precise amount of microbial and/or extracted lipid, preferably phospholipid, in a composition or food, beverage or feedstuff of the present invention may be varied depending on, for example, the identity of the microbial, the form and moisture content of the microbial biomass, the total lipid or phospholipid content and fatty acid composition of the total fatty acid content or of the polar lipid contained in the microbial biomass or extract thereof, the intensity of the desired flavour and/or aroma and the intended use of the composition. In some embodiments, the compositions of the present invention comprise per gram of dry compositions or slurries, or per ml in the case of liquid compositions, at least about 25 mg microbial biomass, in particular at least about 50 mg, preferably at least about 60 mg, more preferably at least about 70 mg microbial biomass, for example dry biomass. In particular embodiments, the compositions of the present invention comprise from about 25 mg to about 250 mg microbial biomass, for example from about 25 mg to about 200 mg microbial biomass, for example dry biomass. In particular embodiments, the compositions of the present invention comprise from about 25 mg to about 150 mg, for example from about 50 mg to about 150 mg dry biomass. In particularly preferred embodiments, the present invention provides from about 50 mg to about 100 mg dry biomass, for example about 75 mg dry biomass. According to some embodiments, the compositions of the present invention comprise from about 50 mg to about 200 mg, preferably from about 50 mg to about 150 mg wet biomass. According to some particular embodiments, the compositions of the present invention comprise from about 75 mg to about 125 mg wet biomass.

According to some embodiments, the compositions may comprise per gram of dry compositions or slurries, or per mL in the case of liquid compositions, for example, at least about 5 mg of lipid, preferably phospholipid, extracted from microbes, for example at least about 10 mg or at least about 15 mg of lipid, preferably phospholipid, extracted from the microbes. According to some embodiments, the composition comprises from about 10 mg to about 100 mg, from about 10 mg to about 80 mg, from about 10 to about 70 mg, from about 10 to 60 mg, particularly preferably about 10 to about 50 mg lipid, preferably phospholipid, extracted from the microbes. According to some embodiments, the compositions of the present invention provide at least about 15 mg, for example at least about 20 mg lipid, preferably phospholipid, extracted from the microbes. According to some embodiments, the food, feedstuffs or beverages may comprise per gram of dry compositions or slurries, or per mL in the case of liquid compositions, for example, at least about 0.1 mg of lipid, preferably phospholipid, extracted from microbes, for example at least about 0.2 mg, 0.3 mg, 0.4 mg, 0.5 mg, 0.6 mg, 0.7 mg, 0.8 mg, 0.9 mg, 1 mg, 1.5 mg, 2 mg, 3 mg, 4 mg, 5 mg, 6 mg, 7 mg, 8 mg, 9 mg or at least about 10 mg of lipid, preferably phospholipid, extracted from the microbes. According to some embodiments, the composition comprises from about 0.1 mg to about 100 mg, 0.5 mg to about 80 mg, from about 1 mg to about 50 mg, from about 1 mg to about 30 mg, from about 5 mg to 60 mg, or from about 5 mg to about 30 mg lipid, preferably phospholipid, extracted from the microbes.

In some embodiments, the compositions comprise per gram of dry composition or slurry, or per ml in the case of liquid compositions, at least about 25 mg microbial biomass, such as dry biomass, and at least about 5 mg lipid, preferably phospholipids, extracted from the microbes. In some embodiments, the compositions of the present invention comprise at least about 70 mg microbial biomass, such as dry biomass, and at least about 10 mg of lipid, preferably phospholipids, extracted from the microbes. In some embodiments, the compositions comprise from about 25 mg to about 150 mg dry microbial biomass and from about 10 mg to about 100 mg lipid, preferably phospholipids, extracted from the microbes; for example, from about 50 mg to about 100 mg microbial dry biomass and from about 15 mg to about 50 mg lipid, preferably phospholipids, extracted from the microbes. In some embodiments, the compositions comprise from about 50 mg to about 150 mg microbial wet biomass, and from about 10 mg to about 100 mg lipid, preferably phospholipids, extracted from the microbes; for example, from about 75 mg to about 125 mg microbial wet biomass, and from about 15 mg to about 50 mg lipid, preferably phospholipids, extracted from the microbes.

Compositions, foods, feedstuffs and beverages of the present invention comprise one or more sugars, sugar alcohols, sugar acids, or sugar derivatives, such as in an amount sufficient to facilitate the production of meat-like or meat-associated aroma compounds. Suitable sugars, sugar alcohols, sugar acids or sugar derivatives will be well known to a person skilled in the art. In this context, the sugars, sugar alcohols, sugar acids, or sugar derivatives are suitable for use in Maillard reactions for food, beverage or feedstuff uses. In this context, the sugars, sugar alcohols, sugar acids, or sugar derivatives are a component of the compositions of the invention separate to the microbial biomass or a component thereof and the amino acids or derivatives or salts thereof, even if the microbial biomass or component thereof itself comprises sugars, sugar alcohols, sugar acids or sugar derivatives. Suitable sugars, sugar alcohols, sugar acids, and sugar derivatives include glucose, fructose, ribose, sucrose, arabinose, glucose-6-phosphate, fructose-6-phosphate, fructose 1,6-diphosphate, inositol, maltose, molasses, maltodextrin, glycogen, galactose, lactose, ribitol, gluconic acid and glucuronic acid, amylose, amylopectin, or xylose. In particularly preferred embodiments, the one or more sugars, sugar alcohols, sugar acids or sugar derivatives comprise one or more of ribose, glucose (dextrose), a combination of glucose and fructose, and xylose. In particularly preferred embodiments, the compositions of the present invention comprise ribose; in the Examples of the present application, ribose was found, in some instances, to provide compositions which produce a more meaty flavour and/or aroma than compositions containing glucose alone as the sugar. In particular embodiments, the compositions of the present invention comprise both glucose and ribose; in the Examples of the present application, ribose and glucose in combination were found, in some instances, to provide compositions which produce a more meaty flavour and/or aroma than compositions containing ribose alone.

According to some embodiments, the one or more sugars, sugar alcohols, sugar acids or sugar derivatives are present in the composition at a total amount of, per kg of dry composition or slurry, or per L in the case of liquid compositions, from about from about 5 mmol to about 200 mmol, for example from about 5 mmol to about 100 mmol, for example from about 5 mmol to about 80 mmol, for example from about 5 mmol to about 70 mmol, for example from about 10 mmol to about 70 mmol, for example from about 15 mmol to about 70 mmol, for example from about 30 mmol to about 60 mmoll, the amount being measured based on the weight or volume of the composition excluding/before addition of the microbial biomass and/or lipids, preferably phospholipids, extracted from microbes. In some embodiments, the one or more sugars, sugar alcohols, sugar acids, or sugar derivatives are present in the composition at an amount of per kg of dry compositions or slurries, or per L in the case of liquid compositions, of at least about 5 mmol, for example at least about 10 mmol, for example at least about 15 mmol, for example at least about 20 mmol, the amount being measured based on the weight or volume of the composition excluding/before addition of biomass and/or extracted lipids. In preferred embodiments, the one or more sugars, sugar alcohols, sugar acids, or sugar derivatives comprise ribose and/or glucose.

In some embodiments, the one or more sugars, sugar alcohols, sugar acids or sugar derivatives are present in the food, feedstuff or beverage at a total amount of, per kg of dry composition or slurry, or per L in the case of liquid foods (e.g. beverages), from about 0.1 mmol to about 100 mmol, from about 0.5 mmol to about 30 mmol, from about 1 mmol to about 20 mmol, from about 1 mmol to about 10 mmol, from about 7 mmol to about 20 mmol, from about 7 mmol to about 15 mmol, the amount being measured based on the weight or volume of the food, feedstuff or beverage excluding/before addition of the microbial biomass and/or lipids, preferably phospholipids, extracted from microbes. In preferred embodiments, the one or more sugars, sugar alcohols, sugar acids, or sugar derivatives comprise ribose and/or glucose.

A sugar “derivative” as used herein means sugars which are modified from a naturally occurring sugar, for example by modification of substituents such as hydroxyl groups. For example, sugar derivatives may have been modified to include alternative substituents such as amino groups, acid groups, phosphate groups, acetate groups etc. Sugar derivatives include, but are not limited to, amino sugars, deoxy sugars, glycosylamines, and sugar phosphates.

In embodiments, compositions, food, feedstuff and beverages of the present invention comprise one or more amino acids or derivatives or salts thereof, such as in an amount sufficient to facilitate the production of meat-like or meat-associated aroma compounds. In this context, the amino acids or derivatives or salts thereof are suitable for use in Maillard reactions for a food, beverage or feed use. In this context, the amino acids or derivatives or salts thereof are a component separate to the microbial biomass or a component thereof and the sugar, sugar alcohol, sugar acid or sugar derivative, even if the microbial biomass or component thereof itself comprises amino acids or derivatives or salts thereof. In particular embodiments, the one or more amino acids or derivatives or salts thereof contain a free amino group. Thus, in some embodiments reference to an amino acid or derivative means a free amino acid that is not present in the context of a peptide or protein. Suitable amino acids and derivatives thereof include cysteine, cystine, a cysteine sulfoxide, allicin, selenocysteine, methionine, isoleucine, leucine, lysine, phenylalanine, threonine, tryptophan, 5-hydroxytryptophan, valine, arginine, histidine, alanine, asparagine, aspartate, glutamate or glutamic acid, glutamine, glycine, proline, serine, and tyrosine. In particularly preferred embodiments, the amino acid is cysteine and/or cystine. In some preferred embodiments, the composition, food, feedstuff or beverage comprises glutamic acid or a salt thereof; in the Examples of the present application, the presence of glutamic acid in some instances was found to provide a more meaty/fishy flavour and/or aroma. In some particularly preferred embodiments, the composition, food, feedstuff or beverage comprises glutamic acid or a salt thereof in addition to one or more other amino acids or derivatives or salts thereof; for example, the compositions, foods, feedstuffs or beverages may comprise glutamic acid or a salt thereof and cysteine or a salt thereof. In preferred embodiments, the one or more amino acids or derivatives or salt thereof comprises a sulfur-containing amino acid or salt. Salts of amino acids which are suitable for human or animal consumption and therefore for incorporation into compositions of the present invention will be familiar to and readily selected by a person skilled in the art.

An amino acid “derivative” as used herein means amino acids which include a chemical modification, for example by introducing a group in a side chain of an amino acid, such as a nitro group in tyrosine or iodine in tyrosine, by conversion of a free carboxylic group to an ester group or to an amide group, by converting an amino group to an amide by acylation, by acylating a hydroxy group rendering an ester, by alkylation of a primary amine rendering a secondary amine, or linkage of a hydrophilic moiety to an amino acid side chain. Other derivatives may be obtained by oxidation or reduction of the side-chains of the amino acid. Modification of an amino acid may also include derivation of an amino acid by the addition and/or removal of chemical groups to/from the amino acid, and may include use of an amino amino acid analog such as a phosphorylated amino acid or a non-naturally occurring amino acid such as a N-alkylated amino acid (e.g. N-methyl amino acid), D-amino acid, β-amino acid or γ-amino acid. Exemplary derivatives may include derivatives obtained by attachment of a derivative moiety, i.e. a substituent group, to an amino acid. The term “derivative” in the context of amino acids will be readily understood by a skilled person.

According to some embodiments, each of the one or more amino acids or derivatives or salts thereof are present in the composition at a total amount of, per kg of dry composition or slurry, or per L in the case of liquid compositions, from about 5 mmol to about 200 mmol, for example from about 5 mmol to about 100 mmol, for example from about 5 mmol to about 80 mmol, for example from about 5 mmol to about 70 mmol, for example from about 10 mmol to about 70 mmol, for example from about 15 mmol to about 70 mmol, for example from about 30 mmol to about 60 mmol, the amount being calculated based on the weight or volume of the composition excluding/before addition of microbial biomass and/or lipids, preferably phospholipids, extracted from the microbes. In some embodiments, the one or more amino acids or derivatives or salts thereof are present in the composition at an amount of per kg of dry compositions or slurries, or per L in the case of liquid compositions, of at least about 5 mmol, for example at least about 10 mmol, for example at least about 15 mmol, for example at least about 20 mmol, the amount being calculated based on the weight or volume of the composition excluding/before addition of microbial biomass and/or lipids, preferably phospholipids, extracted from the microbes. In preferred such embodiments, the one or more amino acids comprises cysteine and/or cystine.

According to some embodiments, each of the one or more amino acids or derivatives or salts thereof are present in the food, feedstuff or beverage at a total amount of, per kg of dry composition or slurry, or per L in the case of liquid foods (e.g. beverages), from about 0.5 mmol to about 40 mmol, about 0.5 mmol to about 30 mmol, about 1 mmol to about 10 mmol, about 1.5 mmol to about 10 mmol, about 0.5 to about 5 mmol, about 1 mmol to about 5 mmol, or about 5 to about 10 mmol the amount being calculated based on the weight or volume of the food, feedstuff or beverage excluding/before addition of microbial biomass and/or lipids, preferably phospholipids, extracted from the microbes. In preferred embodiments, the one or more amino acids comprises cysteine and/or cystine.

The one or more sugars, sugar alcohols, sugar acids, or sugar derivatives and one or more amino acids or derivatives or salts thereof are present in the compositions of the present disclosure or the food products, beverage products or feedstuffs of the present disclosure in amounts sufficient to product food-like aromas, such as meat-like aromas, when heat is applied to the compositions, food products, beverage products or feedstuffs. In particular embodiments, the one or more sugars, sugar alcohols, sugar acids, or sugar derivatives and one or more amino acids or derivatives or salts thereof are present in the compositions of the present disclosure or the food products, beverage products or feedstuffs of the present disclosure in amounts sufficient to produce one or more volatile compounds selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, and 1,3,5-thitriane, for example two or more, three or more, four or more or five or more of the aforesaid compounds when heat is applied to the composition, food product, beverage product or feedstuff. In some particular embodiments, the one or more sugars, sugar alcohols, sugar acids, or sugar derivatives and one or more amino acids or derivatives or salts thereof are present in the compositions of the present disclosure or the food products, beverage products or feedstuffs of the present disclosure in amounts sufficient to produce one or more (e.g. 2, 3, 4, 5, 6, 7, 8 or 9) volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol when heat is applied to the composition, food product, beverage product or feedstuff. In other embodiments, one or more (e.g. 2, 3, 4 or 5) volatile compound(s) selected from 1-pentanal, 3-octanone, 2-octen-1-ol, 1-nonanol and 1-octanol, and optionally 1,3-bis(1,1-dimethylethyl)-benzene are produced.

In some embodiments, the composition of the invention comprises glutamic acid or a salt or derivative thereof in addition to one or more other amino acids or derivatives or salts thereof, and the glutamic acid is present in an amount of, per kg of dry composition or slurry, or per L in the case of liquid compositions, from about 2 mmol to about 100 mmol, for example 2 mmol to about 50 mmol, for example from about 2 mmol to about 40 mmol, for example from about 2 mmol to about 40 mmol, for example from about 5 mmol to about 40 mmol, for example from about 5 mmol to about 30 mmol, the amount being calculated based on the volume of the composition excluding/before addition of microbial biomass and/or lipids, preferably phospholipids, extracted from the microbes. In some embodiments, the glutamic acid or salt thereof is present in an amount of, per kg of dry compositions or slurries, or per L in the case of liquid compositions, at least about 1 mmol, for example at least about 2 mmol, for example at least about 3 mmol, for example at least about 4 mmol, for example at least about 5 mmol, for example at least about 7 mmol, for example at least about 10 mmol, the amount being measured based on the weight or volume of the composition excluding/before addition of biomass and/or extracted lipids. In some embodiments, the glutamic acid salt is monosodium glutamate.

In some embodiments, the food, feedstuff or beverage of the invention comprises glutamic acid or a salt or derivative thereof in addition to one or more other amino acids or derivatives or salts thereof, and the glutamic acid is present in an amount of, per kg of dry composition or slurry, or per L in the case of liquid compositions (e.g. beverages), from about 0.1 mmol to about 20 mmol, about 0.3 mmol to about 15 mmol, about 0.5 mmol to about 10 mmol, about 0.5 mmol to about 5 mmol, or about 1 mmol to about 5 mmol, the amount being calculated based on the volume of the food, feedstuff or beverage excluding/before addition of microbial biomass and/or lipids, preferably phospholipids, extracted from the microbes.

In some embodiments, the composition comprises glutamic acid or a salt thereof and a further amino acid or salt or derivative thereof selected from cysteine and cystine, wherein the glutamic acid or salt thereof is present in an amount of, per kg of dry compositions or slurries, or per L in the case of liquid compositions, from about 2 mmol to about 100 mmol, for example 2 mmol to about 50 mmol, for example from about 2 mmol to about 40 mmol, for example from about 2 mmol to about 40 mmol, for example from about 5 mmol to about 40 mmol, for example from about 5 mmol to about 30 mmol, and the cysteine or cystine is present in an amount of from about 5 mmol to about 200 mmol 5 mmol to about 100 mmol, for example from about 5 mmol to about 80 mmol, for example from about 5 mmol to about 70 mmol, for example from about 10 mmol to about 70 mmol, for example from about 15 mmol to about 70 mmol, for example from about 30 mmol to about 60 mmol, the amount being calculated based on the weight or volume of the composition excluding/before addition of biomass and/or extracted lipid. In some embodiments, the composition comprises glutamic acid or a salt thereof and a further amino acid or salt or derivative thereof selected from cysteine and cystine, wherein the glutamic acid or salt thereof is present in an amount of, per kg of dry compositions or slurries, or per L in the case of liquid compositions, at least about 1 mmol, for example at least about 2 mmol, for example at least about 3 mmol, for example at least about 4 mmol, for example at least about 5 mmol, for example at least about 7 mmol, for example at least about 10 mmol, and the cysteine or cystine is present in an amount of at least about 5 mmol, for example at least about 10 mmol, for example at least about 15 mmol, for example at least about 20 mmol, the amount being calculated based on the weight or volume of the composition excluding/before addition of biomass and/or extracted lipid comprising phospholipids.

Preferred compositions, foods, feedstuffs or beverages of the present invention comprise iron as an additional, separate component. Iron may enhance the meaty flavour and/or aromas produced by compositions, foods, feedstuffs or beverages of the present invention. In some embodiments, the iron is in the form of an iron salt, preferably a ferrous salt. Any iron salt suitable for consumption may be used, and such salts will be familiar to a person skilled in the art, for example a chelated form of iron. In some embodiments, the source of iron is iron (II) fumarate. Iron (II) fumarate is available, for example, as iron tablets from APOHEALTH Pty Ltd (NSW, Australia). The source of iron is a component other than the microbial biomass or a component thereof, even if the microbial biomass or component thereof itself comprises iron.

In particular embodiments, the compositions of the present invention comprise iron in an amount equivalent to, per kg of dry composition or slurry, or per L in the case of liquid compositions, up to about 100 mg of elemental iron, up to about 50 mg, about 20 to about 50 mg, or about 30 to about 40 mg, the amount being calculated based on the volume of the composition excluding/before addition of microbial biomass and/or lipids, preferably phospholipids, extracted from the microbes.

In particularly preferred embodiments, the compositions, foods and feedstuffs of the present invention comprise an aqueous component. The presence of some moisture in the compositions facilitates production of food-like flavour and/or aromas upon heating. In some embodiments, the aqueous component comprises, for example, an aqueous buffer such as a phosphate buffer. In particular embodiments, the compositions of the present invention comprise an aqueous component aside from any water contained incidentally in other components, such as any moisture present in microbial biomass. Compositions of the present invention are preferably not dry or substantially dry, having less than 10% moisture by weight. In one embodiment, the composition is a dry composition. In another embodiment, the composition is a liquid composition. In one embodiment, the composition is in the form of a powder, solution, suspension, slurry or emulsion. In some embodiments, the composition is provided as a composition excluding an aqueous component (i.e. a dry composition), and an aqueous component is added to the composition prior to heating.

In some embodiments, compositions of the present invention may further comprise an aqueous buffer. A buffer maintains the pH of the composition and provides moisture to the composition which, as discussed above, facilitates production of food-like flavour and/or aromas upon heating. In some embodiments, the buffer may be a phosphate buffer. In some embodiments, the buffer may be a buffer at a pH of from about 5.0 to about 7, for example from about 5 to about 6, for example at about 5.3 or about 6.0. In particular embodiments, the buffer is a phosphate buffer at a pH of about 6.0.

The compositions, foods, feedstuffs or beverages of the present invention may further comprise one or more additional components. Such components may be flavour precursors, for example intended to be involved with Maillard reactions occurring when the composition is heated. For example, such additional components may include oils, for example vegetable oils, free fatty acids, alpha-hydroxy acids, dicarboxylic acids, nucleosides, nucleotides, vitamins, peptides, protein hydrolysates, extracts, phospholipids, lecithin, and organic molecules.

In some embodiments, the compositions, foods, feedstuffs or beverages further comprise thiamine. Thiamine may enhance the meaty aroma and/or flavour produced by compositions of the present invention. In some embodiments, thiamine may be present in the composition, per kg of dry composition or slurry, or per L in the case of liquid compositions, in an amount of from about 0.5 to about 5 mmol, about 1 to about 4 mmol, or about 1 to about 3 mmol, or from at least about 0.2 mmol, for example at least about 0.3 mmol, for example at least about 0.4 mmol, for example at least about 0.5 mmol, for example at least about 0.7 mmol. In particular embodiments, thiamine is present in an amount of from about 1.5 mmol to about 2.5 mmol, for example about 2 mmol, or the amount being calculated based on the weight or volume of the composition excluding/before addition of microbial biomass and/or lipids, preferably phospholipids, extracted from the microbes. In some embodiments, thiamine may be present in the feedstuffs or beverages, per kg of dry composition or slurry, or per L in the case of liquid compositions (e.g. beverages), in an amount of from about 0.1 to about 5 mmol, about 0.1 to about 1 mmol, about 0.5 to about 5 mmol, or about 1 to about 3 mmol, the amount being calculated based on the weight or volume of the food, feedstuff or beverage excluding/before addition of microbial biomass and/or lipids, preferably phospholipids, extracted from the microbes.

In some embodiments, the compositions, foods, feedstuffs or beverages further comprise a yeast extract. In the art of food science, a “yeast extract” is generally understood to refer to a water-soluble portion of autolyzed yeast and is available commercially from various suppliers; see, for example Sigma Aldrich, Catalog No. Y1625 Yeast Extract. A yeast extract does not contain yeast whole cell biomass. The presence of a yeast extract may enhance meaty aromas and/or flavours produced by the composition when heated. The yeast extract may be a general unflavoured yeast extract, or may be, for example, a beef flavoured or roast chicken skin flavoured yeast extract. In some embodiments, the composition is suitable for producing food-like aromas and/or flavours which are meat-like aromas and/or flavours, and the composition comprises a yeast extract. The presence of a yeast extract may enhance meaty aromas and/or flavours produced by compositions of the present invention, as observed in the Examples below.

In some embodiments, the yeast extract is present in the composition in an amount of, per kg of dry composition or slurry, or per L in the case of liquid compositions, from about 100 mg to about 200 gm, or about 200 mg to about 100 gm, or from about 10 g to about 200 g, for example from about 15 g to about 200g, for example from about 20 g to about 200g, for example from about 30 g to about 200g, for example from about 40 g to about 200g, for example from about 50 g to about 200g, for example from about 50 g to about 180 g, for example from about 60 g to about 180 g, the amount being calculated based on the volume of the composition excluding/before addition of microbial biomass and/or phospholipids extracted from the microbes. In some embodiments, the yeast extract is present in the composition in an amount of, per kg of dry compositions or slurries, or per L in the case of liquid compositions, at least about 5g, for example at least about 7 g, for example at least about 10 g, for example at least about 15 g, for example at least about 20 g, for example at least about 25 g, for example at least about 30 g, for example at least about 40 g, for example at least about 50 g, for example at least about 60 g. In particular embodiments, the yeast extract is present in the composition in an amount of, per kg of dry compositions or slurries, or per L in the case of liquid compositions, at least about 30 g.

In some embodiments, the composition, food, feedstuff or beverage does not comprise a yeast extract. Since the presence of a yeast extract may enhance meaty aromas and/or flavours produced by the composition, food, feedstuff or beverage, a yeast extract maybe omitted when, for example, an alternative food-like flavour and/or aroma is desired, such as a vegetable or herby aroma and/or flavour. The absence of a yeast extract may reduce the potential masking of the desired aroma and/or flavour such as a vegetable-like aroma and/or flavour by meat-like aromas and/or flavours enhanced by the presence of a yeast extract. Accordingly, in some embodiments, the food-like aroma and/or flavour is a fish-like aroma and/or flavour, a vegetable, and/or a herby aroma and/or flavour, and the composition, food, feedstuff or beverage does not comprise a yeast extract.

In some embodiments, the compositions, foods, feedstuffs or beverages further comprise one or more herbs and/or spices. As demonstrated in the Examples below, compositions comprising herbs, such as for example Fenugreek (Trigonella foenum-graecum), were found in some instances to enhance vegetable, soupy and/or herby flavour and/or aromas produced by the compositions of the present invention. These herby, vegetable and/or soupy flavour and/or aromas may partially or completely mask meaty/fishy aromas and/or flavours in some embodiments, allowing adjustment of overall aromas and/or flavours produced by compositions of the present invention. A herb and/or spice as used herein refers to a plant part or extract possessing aromatic properties which is suitable for use in foods or beverages. Typically, a herb is understood to refer to leafy, green or flowering parts of a plant, whilst a spice is typically understood to refer to other parts of a plant, usually dried, including seeds, bark, roots and fruit. The herb or spice may be in the form of whole plant parts, or chopped, ground or rolled plant parts, or dried, for example as a powder. In particular embodiments, the one or more herbs and/or spices comprise Fenugreek. Fenugreek has also been claimed to contain several bioactive components and can bring health benefits to consumers. In some embodiments, the one or more herbs and/or spices comprise Fenugreek leaf.

In an embodiment, the composition, food, feedstuff or beverage of the invention comprises: (a) microbial biomass containing phospholipids and/or phospholipids extracted from the microbes, (b) glucose and/or ribose, (c) cysteine and/or cystine, (d) a source of iron, for example an iron salt, (e) glutamic acid or a salt thereof, (f) thiamine, (g) an aqueous component, for example an aqueous buffer such as a phosphate buffer, for example having a pH of from about 5 to about 6, for example of about 5.3 or about 6.0, and (h) optionally a yeast extract. In an embodiment, the composition comprises (b) ribose and (c) cysteine.

The compositions, foods, feedstuffs or beverages of the present invention produce a food-like flavour and/or aroma, preferably a meat-like flavour and/or aroma, when heated. Heating refers to increasing the temperature of the composition, for example to above room temperature, to any temperature and for any amount of time sufficient to produce food-like flavour and/or aromas. In this context, the temperature is raised high enough and long enough for Maillard reactions to occur between amino groups and sugars in the composition, with additional reactions occurring with lipids, preferably phospholipids, or breakdown products thereof, in the composition, food, feedstuff or beverage to produce the food-like flavour and/or aromas. Selection of a suitable temperature and period of time for the heating step is readily carried out by the skilled person. As used herein, “heated” or “heating” or similar is to be understood as meaning heating under conditions sufficient for producing a food-like aroma, unless otherwise specified. The heat may be applied to the composition of the invention prior to it being contacted with the food product, or after the application to the food product, or both. Such heating of the composition, the food product with the composition or the food, feedstuff or beverage of the invention, may take place for example in an oven, frypan, wok or similar, or in a barbeque. Whilst the precise temperature to which a composition, food, feedstuff or beverage should be heated to produce a food-like flavour and/or aroma, preferably a meat-like flavour and/or aroma, may vary depending on, for example, the precise composition, food, feedstuff or beverage and the time for which the composition, food, feedstuff or beverage is heated and the amount of composition, food, feedstuff or beverage being heated, in some embodiments, the compositions or food products containing the compositions produce a food-like flavour and/or aroma when heated to a temperature of for example at least about 100° C., at least about 110° C., at least about 120° C., at least about 130° C., or at least about 140° C. In this context, the temperature should not be that high that the food product burns or has a burnt flavour and/or aroma. In particular embodiments, the compositions, food, feedstuff or beverage produce a food-like flavour and/or aroma when heated to about 140° C.

Similarly, the compositions and food products of the present invention produce a food-like flavour and/or aroma, preferably a meat-like flavour and/or aroma when heated for varying amounts of time, depending on, for example, the temperature to which the compositions are heated, the precise nature of the composition and the amount of composition being heated. Nonetheless, in some embodiments the composition, food, feedstuff or beverage may produce a food-like flavour and/or aroma when heated for at least 5 or at least 10 minutes, for example at least 15 minutes, for at least about 30 minutes, or at least about 45 minutes. In some embodiments, the compositions, food, feedstuff or beverage may produce a food like flavour and/or aroma when heated for at least about 1 hour, for example about 1 hour. Preferably, the heat is applied for a length of time whereby a burnt flavour and/or aroma is not produced, as is understood by a person of skill in the art.

In an embodiment, the composition, food, feedstuff or beverage of the present invention produces a food-like flavour and/or aroma, preferably a meat-like flavour and/or aroma, when heated for at least 5 or at least 10 minutes at a temperature of at least about 100° C., for at least 30 minutes at a temperature of at least about 100° C., for at least 30 minutes at a temperature of at least about 120° C., for at least 30 minutes at a temperature of at least about 130° C., for at least 1 hour at a temperature of at least about 130° C., or for at least 1 hour at a temperature of at least about 140° C. In a preferred embodiment, the composition, food, feedstuff or beverage produces a food-like flavour and/or aroma when heated for about 1 hour at about 140° C.

It will be appreciated that compositions, foods, feedstuffs or beverages of the present invention may, according to some embodiments, produce food-like flavours and/or aromas when heated to temperatures and for time periods different to those outlined above, but that, in some embodiments, stronger and/or more desirable food-like flavours and/or aromas may be produced when the compositions are heated to the temperatures discussed above and/or for the time periods discussed above.

The food-like flavours and/or aromas produced by compositions, foods, feedstuffs or beverages of the present invention may, according to some preferred embodiments, include a meat-like flavour and/or aroma. In particular embodiments, the food-like flavour and/or aroma may be an aroma of cooked meat or a meat-based food. For example, the food-like flavour and/or aroma may be of beef, steak, chicken, for example roasted chicken or chicken skin, pork, lamb, duck, venison, chicken or other meat soup, meat broth, liver, or generally “meaty”. Such aromas are typically detected by human volunteers, for example by a qualified sensory panel. In this context, a composition, food, feedstuff or beverage is said to produce a food-like or meat-like flavour and/or aroma when at least one third, for example at least one half, of the number of volunteers on a tasting/smelling panel detect a food-like or meat-like flavour and/or aroma in a double-blind test of the composition, food or beverage. In analogous fashion, a food product or beverage comprising a composition of the invention has an increased food-like or meat-like flavour and/or aroma, when at least one third, for example at least one half, of the number of volunteers on a tasting/smelling panel detect an increased food-like or meat-like flavour and/or aroma relative to a corresponding food product or beverage lacking the composition of the invention, in a double-blind test. It will be appreciated that, in some instances, there will be a degree of variability in how various flavours and/or aromas are perceived by different subjects experiencing those aromas, and subjects may describe precise flavour and/or aromas slightly differently. In an embodiment, the number of volunteers is at least 6, for example at least 10, at least 25, at least 50, or between 6 and 50.

In some embodiments, heating of the composition, food, feedstuff or beverage produces one or more (e.g. volatile compounds selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, and 1,3,5-thitriane, for example two or more, three or more, four or more or five or more of the aforesaid compounds. In some particular embodiments, heating produces one or more volatile compounds selected from 2-heptanone, 3-octanone, 2,3-octanedione, 1-pentanol, 1-hexanol, 2-ethyl-1-hexanol, 1-octanol, trans-2-octen-1-ol and 1-nonanol.

The food-like flavours and/or aromas produced by compositions, foods, feedstuffs or beverages of the present invention may, according to some embodiments, include a fish-like flavour and/or aroma, for example a cooked fish flavour and/or aroma, for example a fried fish flavour and/or aroma a vegetable and/or herbal flavour and/or aroma, for example a cooked vegetable and/or herby flavour and/or aroma, for example a soup, mushroom, onion, vegetable, herbal or roasted vegetable flavour and/or aroma. In some embodiments, the composition, food, feedstuff or beverage includes ribose and the food-like flavour and/or aroma includes a meaty flavour and/or aroma, for example cooked meat-like flavour and/or aroma, and/or a fishy flavour and/or aroma, for example a cooked or fried fish-like flavour and/or aroma.

In some embodiments, the composition, food, feedstuff or beverage includes glutamic acid, for example glutamic acid in addition to a further amino acid or salt or derivative thereof such as cysteine, and the food-like flavour and/or aroma includes a meaty flavour and/or aroma, for example cooked meat-like, and/or a fishy flavour and/or aroma, for example a cooked or fried fish-like flavour and/or aroma.

In some embodiments, the composition, food, feedstuff or beverage includes a yeast extract and the food-like flavour and/or aroma includes a meaty flavour and/or aroma, for example cooked meat-like flavour and/or aroma. In some embodiments, the composition does not include a yeast extract and the food-like flavour and/or aroma includes a fish-like flavour and/or aroma, for example cooked fish or fried fish-like, vegetable and/or herby aroma and/or flavour.

In some preferred embodiments, the microbe is Saccharomyces spp., Yarrowia spp., Mortierella spp., or Mucor spp., for example Saccharomyces cerevisiae, Yarrowia lipolytica, Mortierella alpina or Mucor hiemalis, for example Saccharomyces cerevisiae strain D5A Yarrowia lipolytica strain W29, Mortierella alpina or Mucor hiemalis, and the food-like flavour and/or aroma includes a meat-like flavour and/or aroma, for example a chicken-like flavour and/or aroma for example a cooked chicken flavour and/or aroma, for example a roast chicken, chicken skin or chicken broth flavour and/or aroma. In preferred embodiments, the microbial biomass is of a species that is Mortierella spp., for example Mortierella alpina, and the food-like flavour and/or aroma includes a beef-like flavour and/or aroma.

In some embodiments, the composition, food, feedstuff or beverage includes one or more herbs and/or spices, for example fenugreek, for example fenugreek leaf, and the food-like flavour and/or aroma includes a vegetable, soupy and/or herby flavour and/or aroma.

It will be appreciated that, in some instances, there will be a degree of variability in how various flavours and aromas are perceived by different subjects experiencing those aromas, and subjects may describe precise flavours and aromas slightly differently.

In particular embodiments, compositions, foods, feedstuffs or beverages of the present invention may produce food-like flavours as well as food-like aromas. Such food-like flavours may be flavours corresponding to the food-like aromas disclosed herein. As such, reference to aromas herein may be understood, according to certain aspects, to instead also refer to aromas and/or flavours where appropriate, and vice versa.

The compositions, foods, feedstuffs or beverages of the present invention are suitable for human or animal consumption, typically at least human consumption.

In some embodiments, the composition of the present invention is incorporated into the food or beverage product or feedstuff prior to or during heating, such that when the food or beverage product is heated, for example during cooking, the composition produces the associated food-like aromas by way of a Maillard and associated reactions. In some embodiments, the composition of the present invention is heated prior to incorporation in or addition to a food or beverage product or feedstuff.

The present invention further relates to a method of producing a food product, beverage product or feedstuff comprising combining a composition of the present invention with one or more additional consumable ingredients. The present invention further relates to a method of producing a food product, beverage product or feedstuff comprising combining a microbial lipid of the present invention with an animo acid and a sugar and one or more additional consumable ingredients. Each of the embodiments described above in the context of the compositions of the invention also apply to the foods, beverages and feedstuffs of the invention, to methods of making the same, and to uses of the foods, beverages and feedstuffs. Suitable additional ingredients which may be included in such food products, beverage products or feedstuffs are discussed below. For example, the composition can be combined with the other consumable ingredient by mixing, applying it to the surface of the other ingredient, or by soaking/marinating the other ingredient. In an embodiment, the food, feedstuff or beverage product is prepared by (a) heating a composition of the invention and (b) mixing the products from (a) with other food, feedstuff or beverage consumable ingredients, or by (a) mixing a composition of the present invention with other food, feedstuff or beverage consumable ingredients and (b) heating the mixture resulting from (a).

The food product, beverage product or feedstuff may either be in a solid or liquid form, and may be intended to be kept frozen, refrigerated or at room temperature prior to cooking. In some embodiments, the food product, beverage product or feedstuff is provided as a dry product excluding an aqueous component, and an aqueous component (such as water) is added to the composition prior to, during or subsequent to heating, especially prior to heating. The food or beverage product or feedstuff may include edible macronutrients, protein, carbohydrate, vitamins, and/or minerals in amounts desired for a particular use. The amounts of these ingredients will vary depending on whether the composition is intended for use with normal individuals or for use with individuals having specialized needs, such as individuals suffering from metabolic disorders and the like, or for vegetarians or vegans.

According to preferred embodiments, the food or beverage product of the present invention contains no components derived from an animal. In a preferred embodiment, at least some of the ingredients are plant material or material derived from a plant. Such embodiments are advantageously suitable for a vegan or vegetarian diet. In some embodiments, the food or beverage product or feedstuff can be soy-free, wheat-free, yeast-free, MSG-free, and/or free of protein hydrolysis products. The food or beverage product or feedstuff preferably has a food-like taste or aroma, such as a meaty or fishy aroma, as imparted by the composition of the present invention.

### EXAMPLES

### Example 1. Materials and Methods

### Media and Chemicals

YPD medium is a rich medium which contains 10 g/L yeast extract (Sigma Aldrich, Catalog No. Y1625), 20 g/L peptone (Sigma Aldrich, Catalog No. P0556) and 20 g/L glucose (Sigma Aldrich, Catalog No. G7021). YPD plates contain, in addition, 20 g/L agar. SD-Ura medium contained Yeast Synthetic Drop-out Medium (Sigma Catalog No. Y1501).

Chemicals were sourced as follows unless stated otherwise: L-cysteine (Sigma, Catalog No. 168149), D-(−) ribose (Sigma, Catalog No. R7500), thiamine hydrochloride (Sigma, Catalog No. 47858), iron fumarate (Fe²⁺, Apohealth, NSW, Australia; Code #MH/Drugs/25-KD/617), L-glutamic acid monosodium salt hydrate (Sigma, Catalog No. G5889), potassium dihydrogen phosphate (Sigma, Catalog No. 1048731000).

### Media for Larger Scale Cultures

Unless otherwise stated, the medium used for preparing seed cultures for larger scale cultures (2 L or more) was a defined medium (DM), having a base medium (BM) containing 10.64 g/L potassium di-hydrogen orthophosphate (KH₂PO₄), 4.0 g/L di-ammonium hydrogen orthophosphate ((NH₄)₂HPO₄) and 1.7 g/L citric acid (monohydrate). These ingredients were dissolved in about 70% of the required volume of water that had been purified by reverse osmosis, adjusted to pH 6.0 with 2 M NaOH, and made up to the required volume using purified water. The BM was sterilised at 121° C. for 20 min and cooled to room temperature. The following ingredients were then added separately: 30 ml/L of 660 g/L glucose (autoclaved), to a final concentration of 20 g/L, 10 ml/L 1 M magnesium sulphate heptahydrate (autoclaved), 10 ml/L Trace metal solution (see below, filter sterilised), 10 ml/L 15 g/L thiamine hydrochloride (filter sterilised), 3 ml/L 10% (v/v) Sigma Antifoam 204 (autoclaved).

The fermentation medium (FM) for 2 L and 10 L cultures also used the BM as base medium. The required volume was added to the bioreactor and sterilised at 121° C. for a 60 min fluid cycle for an autoclavable bioreactor or 30 min for a steam-in-place bioreactor, and cooled to 31° C. The following ingredients were added, per litre of base medium: 121 ml/L of 660 g/L glucose (autoclaved), giving a final concentration of 80 g/L, 5 ml/L of 1M magnesium sulphate heptahydrate (autoclaved), 5 ml/L of Trace metal solution (see below, filter sterilised), 5 ml/L 15 g/L thiamine hydrochloride (filter sterilised) and 50 ml/L of 200 g/L ammonium chloride (filter sterilised). The glucose, magnesium, trace metal solution and thiamine solution were mixed and added to the bioreactor together. Once the medium was formulated, the pH was checked, normally slightly less than 6.0. A pH controller was used to add ammonia solution to the medium and bring the pH to 6.0.

The Trace metal solution (TM) contained, per litre: 2.0 g CuSO₄·5H₂O, 0.08 g NaI, 3.0 g MnSO₄·H₂O, 0.2 g NaMoO₄·2H₂O, 0.02 g H₃BO₃, 0.5 g CoCl₂·6H₂O, 7.0 g ZnCl₂, 22.0 g FeSO₄·7H₂O, 0.50 g CaSO₄·2H₂O, and 1 ml of sulphuric acid. The reagents were added in the listed order. Addition of the sulphuric acid resulted in dissolution of the calcium sulphate. The trace metal solution was filtered sterilised through a 0.2 μm filter and stored at 2-8° C. in a bottle wrapped in aluminium foil.

One pH control reagent was a phosphoric acid solution (10% w/v), prepared by adding 118 ml of 85% H₃PO₄ to 882 ml of purified water. The solution was sterilised by autoclaving.

The other was an ammonia solution (10% v/v), prepared by adding 330 ml of a 30% ammonia solution to 670 ml of purified water. That solution was assumed to be self-sterilising. An antifoam solution was prepared by mixing 100 ml of Sigma antifoam 204 with 900 ml of purified water, providing a concentration of 10%. The mixture was sterilised by autoclaving.

A feed solution was prepared by adding 134 ml of 200 g/L ammonium chloride which had been filter sterilised to 1 L of 660 g/L glucose, and sterilised by autoclaving.

### Microbial Strains and Cloning Vectors

S. cerevisiae strains INVSc1 (ThermoFisher, Catalog No. C81000) and D5A (ATCC 200062) were used as host strains for experiments on production of lipids including phospholipids. When testing various lipid modification genes in yeast by addition of transgenes, the pYES2 plasmid was used as the base vector for introduction of the genes. INVSc1 and pYES2 were obtained from Invitrogen (Catalog No. V825-20). The genotype of INVSc1 was: MATa his3Δ1 leu2 trp1-289 ura3-52/MATa his3Δ1 leu2 trp1-289 ura3-52, and its phenotype was: His-, Leu-, Trp- and Ura-. The pYES2 vector had unique HindIII and XhoI restriction enzyme sites which were used for insertion of DNA fragments encoding various proteins as described herein. The pYES2 expression vector contained a URA3 gene as a selectable marker gene for introduction into yeast strains that were Ura-, a 2μ origin of replication for high copy maintenance, and an inducible Gall promoter for expression of the protein coding regions in yeast. The plasmid also contained an ampicillin resistance gene for selection in E. coli during cloning experiments.

Several strains of Yarrowia lipolytica were obtained from the American Type Culture Collection (Manassas VA, USA): Strain JM23 (ATCC 90812) having the genotype leu235 lys512 ura318 xpr2::LYS5B, strain IFP29 (ATCC 20460) having the genotype leu235 lys512 ura318 xpr2::LYS5B, and wild-type strain W29 (Casaregola et al., 2000). Strain Y2047 (ATCC PTA-7186; U.S. Pat. No. 7,588,931) and Y2096 (ATCC PTA-7186) were obtained from ATCC.

Escherichia coli strains DH5α and BL21 were obtained from ThermoFisher Scientific (Catalog Nos. 18265017, EC0114).

The fungal strain described herein as yNI0121 (Mucor hiemalis) has been deposited with National Measurement Institute, Port Melbourne, VIC 3207, Australia on 4 Feb. 2021 under the Budapest Treaty and has been designated the following Deposit Number: yNI0121 Deposit Accession number V22/001757. Fungal strains described herein as yNI0125 (Mortierella elongata), yNI0126 (Mortierella sp.), yNI0127 (Mortierella sp.) and yNI0132 (Mortierella alpina) have been deposited with National Measurement Institute, Port Melbourne, VIC 3207, Australia on 12 Oct. 2021 under the Budapest Treaty and have been designated the following Deposit Numbers: yNI0125 Deposit Accession number V21/019953, yNI0126 Deposit Accession number V21/019951, yNI0127 Deposit Accession number V21/019952, and yNI0132 Deposit Accession number V21/019954.

Growth of S. cerevisiae and Y. lipolytica Cultures for Lipid Analysis

To provide an inoculum for cultures for fatty acid production, extraction and analysis, small-scale cultures of Y. lipolytica or S. cerevisiae were grown in 5 ml of YPD medium at 29° C. for 24 h. For experiments, the inoculum culture was diluted into the growth medium having a volume of, for example, 50-2000 ml to an optical density at 600 nm (OD600) of 0.1. Cultures were grown in polypropylene tubes for 10 ml cultures, or glass flasks for larger volumes, the container having a volume at least 5-fold greater than the culture volume. The containers were sealed with 3M micropore surgical tape (Catalog No. 1530-1) tape and incubated in a shaker at a defined temperature of 29° C. unless specified otherwise, at 200 rpm for aeration.

When SD-Ura medium was used, a carbon source such as 2% glycerol or raffinose (w/v) (MP Chemicals, USA, Catalog No. 4010022) was used. Cultures were incubated overnight at 28° C. with shaking for aeration. The inoculum culture was diluted into 10 ml of SD-Ura medium, or other volume as specified, containing 2% (w/v) glycerol or raffinose and 1% tergitol (v/v) (Sigma Aldrich Catalog No. NP40S) medium to provide an initial OD600 of 0.1. The culture in a 50 ml tube or a 250 ml flask was incubated in a shaker at 28° C. at 200 rpm for aeration. The OD600 was checked at time intervals of 15 or 30 min. When the OD600 reached 0.3, exogenous compounds as potential substrates (if any) were added along with 2% galactose for induction of the transgene from the GALI promoter if appropriate.

Larger scale cultures of S. cerevisiae cells at a volume of 3 L were grown for transformants such as pYES2 derivatives. These were inoculated from glycerol stocks. Starter cultures were grown in 10 ml SD-Ura medium containing 2% (w/v) raffinose for two overnights. The cells were transferred into 3 L of SD-Ura medium containing 2% (w/v) raffinose and 1% tergitol (NP-40) to an OD600 of 0.1 and grown at 28° C. with shaking at 200 rpm. The OD600 was checked at time intervals of 15 and 30 min. When the OD600 reached 0.3, galactose was added to a final concentration of 2% (w/v) to induce the transgene. When desired, sodium butyrate was added to cultures to a final concentration of 2 mg/ml. The flasks were then closed loosely with sterile aluminium foil. The cultures were grown in the incubator for 48 hours before harvesting the cells by centrifugation.

Cultures of E. coli were grown from glycerol stocks in 5 ml LB medium for 24 h to provide an inoculum. The culture was diluted into LB medium in polypropylene tubes or glass flasks, to an OD600 of 0.1 and incubated in a shaker at 37° C. at 200 rpm for aeration, unless otherwise specified.

### Feeding Lipid Substrates to the Cells

For substrate feeding experiments, both yeast and bacterial inoculum cultures were diluted into their respective growth media containing 1% tergitol at an OD600 of 0.1 and incubated with shaking for a period of time, typically 2 h. Lipid substrates such as e.g. fatty acids, oil or oil-hydrolysates were then added to the medium and the cultures further incubated for different time periods. Fatty acid substrates were obtained from NuChek Prep: e.g. γ-linolenic acid (GLA, Catalog No. U-63-A), dihomo-γ-linolenic acid (DGLA, Catalog No. U-69-A), arachidonic acid (ARA, Catalog No. U-71-A), docosatetraenoic acid-N6 (DTA, Catalog No. U-83-A), and docosapentaenoic acid-ω6 (DPAω6, Catalog No. U-102-AX). The fatty acid was dissolved in ethanol and provided to the cultures to a final concentration of 0.5 mg/ml. When used, an ARA-containing oil was obtained from Jinan Boss Chemical Industry Co., Ltd (China), having 50% ARA in its total fatty acid content. The oil was dissolved in ethanol and applied to the cultures to a final concentration up to 5.0 mg/ml.

When compounds were added as potential carbon sources (feeding assays), the following compounds were obtained from Sigma Aldrich: ethanolamine (Catalog No. 110167), choline chloride (C7017), myo-inositol (13011), butyric acid (B103500), sodium butyrate (B5887), tributyrin (W222305) or palmitic acid (76119). Butyric acid dissolved in water was provided to S. cerevisiae to a final concentration of up to 2 mg/ml. When provided to Y. lipolytica cultures, butyric acid (B103500) was prepared in 50% glycerol and added to the cultures to a final concentration of 2 mg/ml.

Oil preparations were also provided to some Y. lipolytica cultures: castor oil (Aussie Soap Supplies, AU, Catalog No. SKU: CB100), tributyrin (Sigma Aldrich, Catalog No. W222305) and long chain polyunsaturated fatty acids (GreenOMEGA 3 Capsules, Green nutritionals, AU). These oils were emulsified in 70% NP40 and added to the medium at a final concentration of 2 mg/ml. In this case the NP40 final concentration was 7% (v/v).

### Parameters for 2 L Fermentation

The following parameters were used for a 3 L (total volume) Sartorius Biostat B autoclavable bioreactor with a maximum working volume of 2 L culture. The starting medium volume was 1 L. The initial temperature set point was 31° C., unchanged for the duration of the process. The temperature controller configuration was Minimum: −100%; Maximum: 100%; XP: 4%; TI: 300 sec; TD: 75 sec; Dead: 0.0%; Cascade control using dissolved oxygen controller; Minimum agitator speed: 500 rpm; Maximum agitator speed: 1200 rpm; pH control set point: 6.0; pH controller configuration: Minimum: −100%, Maximum: 100%, XP: 30%, TI 30 sec, TD: 0 sec, Dead: 0.2% (equivalent to 0.02 pH units). The acid and base used for automated pH control were 10% H₃PO₄ and 10% ammonia solution.

The initial dissolved oxygen set point was 30%. The dissolved oxygen (DO) electrode was calibrated after sterilisation and once the medium temperature had stabilised at 31° C. 0% saturation was calibrated using pure nitrogen, a stirrer speed of 100 rpm and nitrogen flow rate at 0.1 L/min, and saturation was established with the stirrer speed set at 500 rpm and air flow rate at 0.5 L/min. For cascade control, a two step cascade used a stirrer followed by gas mix to provide oxygen enrichment of the air flow. Oxygen enrichment was used to reduce the air flow rates and thereby reduce foaming which can have a negative impact on the process, since the yeast cells tended to float on the foam. The airflow was constant at 0.5 L/min, with minimum oxygen enrichment at 0% and maximum oxygen enrichment at 50%. The dissolved oxygen controller configuration was set at: Dead: 0%, Minimum: 0% (510 rpm), Maximum: 100% (1425 rpm), XP: 90%, TI: 50 sec, TD: 0 sec.

For foam control, automatic chemical foam control was achieved with 10% Sigma Antifoam 204, adding 10 ml of 10% (v/v) Sigma Antifoam 204 before inoculation, 20 ml at 7 h post inoculation, and 30 ml added 31 h post inoculation. The foam controller configuration was: Cycle: 10 sec, Pulse: 5 sec, Sensitivity: 04.

The target inoculation OD600 was 0.20, calculated based on the starting volume of base medium, using the secondary seed culture. For fed batch mode, feed with the feed solution commenced 14 h after inoculation with a feed flow rate of 20 ml/h. At the completion of each process, the vessel was drained, and the cells were harvested by centrifugation.

### Parameters for 10 L Fermentation

The same parameters were used for a 15 L Sartorius Biostat C10 steam-in-place bioreactor with a maximum working volume of 10 L culture, with the following differences. To calibrate the dissolved oxygen (DO) electrode, 0% saturation was calibrated using pure nitrogen at a stirrer speed 100 rpm and nitrogen flow rate of 1 L/min, and saturation was established with the stirrer speed set at 500 rpm and air flow rate at 3 L/min. For cascade control, the airflow was constant at 3.0 L/min. The dissolved oxygen controller configuration was set at: HTime Stirrer: 0 min, Dead: 0.5%, Minimum: 34% at 510 rpm, Maximum: 95% at 1425 rpm, XP: 150%, TI: 100 sec, TD: 0 sec, HTime GasMix: 0 min, Dead: 0.5%, Minimum: 0% (no oxygen supplementation), Maximum: 50%, XP: 5%, TI: 200 sec, TD: 0 sec.

As for the 2 L fermentation, the target inoculation OD600 was 0.20, using a secondary seed culture. For fed batch mode, feed with the feed solution commenced 14 h after inoculation with a feed flow rate of 100 ml/h. At the completion of each process, 24 h after inoculation unless otherwise stated, the culture was heat inactivated at 105° C. for 5 min, then cooled to 31° C. before harvesting the cells by centrifugation.

### Seed Culture for Larger Scale Cultures

For a primary seed culture, a frozen glycerol stock of the yeast strain was used to inoculate 100 mL of DM in a plastic baffled 1 L Erlenmeyer flask with a vented cap. This was incubated at 28° C. with shaking at 200 rpm for aeration for 24±2 h. The optical density at 600 nm (OD600) was measured at the end of incubation. A secondary seed culture was prepared by using the primary seed culture to inoculate 500 mL of DM in a plastic baffled 2 L Erlenmeyer flask with a vented cap, to a starting OD600 of 0.04. The second seed culture was incubated at 28° C. with shaking at 200 rpm for 16±2 hours. The OD600 was measured at the end of incubation. This culture was used to inoculate the large scale fermentation.

### Cell Harvesting, Washing and Freeze Drying

Cells from smaller scale cultures were harvested by centrifugation, for example in a 50 ml tube at 4600 g for 15 min, washed twice with 10 ml and finally washed with 1 ml MilliQ water. For the final wash, where a dry cell weight was to be measured, the cell suspension was transferred to a pre-weighed 2 ml Eppendorf tube, centrifuged, and the cell pellet freeze-dried (VirTis Bench Top freeze dryer, SP Scientific) before weighing and lipid extraction. When lipid substrates such as ARA, DGLA, Y-linolenic acid (GLA), butyrate or palmitate were added to the growth medium, cell pellets were washed successively with 1 ml of 1% tergitol (v/v), 1 ml of 0.5% tergitol and a final wash with 1 ml water to remove any remaining substrate from the exterior of the cells and freeze-dried as described above. When an oil was added to the growth medium, cells were harvested by centrifugation as above but the cell pellets were washed successively with 5 ml of 10% tergitol (v/v), 5 ml of 5% tergitol, 5 ml of 1% tergitol, 5 ml of 0.5% tergitol and a final wash with 5 ml water to remove any remaining oil from the exterior of the cells. In some cases, microscopic observation after staining with Bodipy confirmed the absence of oil stained at the cell walls. With the final wash, pellets were transferred to pre-weighed 2 ml Eppendorf tubes and freeze-dried before weighing and lipid extraction.

Lipid Extraction from Yeast Cells

Total cellular lipid was extracted from yeast cells such as S. cerevisiae or Y. lipolytica by using a method modified from Bligh and Dyer (1959). Approximately 50 mg freeze-dried cells were homogenized with 0.6 ml of a mixture of chloroform/methanol (2/1, v/v) with 0.5 g zirconium oxide beads (Catalog No. ZROB05, Next Advance, Inc., USA) in a 2 ml Eppendorf tube using a Bullet Blender Blue (Next Advance, Inc. USA) at speed 6 for 5 min. The mixture was then sonicated in an ultrasonication water bath for 5 min and 0.3 ml 0.1 M KCl was added. The mixture was shaken for 10 min and centrifuged at 10,000 g for 5 min. The lower, organic phase containing lipid was transferred to a glass vial and remaining lipid was extracted from the upper phase containing the cell debris by mixing it with 0.4 ml chloroform for 20 min and centrifugation. The lower phase was collected and combined with the first extract in the glass vial. The solvent was evaporated from the lipid sample under a flow of nitrogen gas and the extracted lipid resuspended in a measured volume of chloroform. If required, the lipid samples were stored at −20° C. until further analysis.

Lipid Extraction from the Larger Biomass

For the extraction of total lipid from a larger biomass, a different method of cell homogenization was used with larger volumes of the solvents, unless otherwise stated. Approximately 1.5 g of freeze-dried cells, distributed amongst six 50 ml Cellstar polypropylene tubes (6× Tube A) (Catalog No. 227261, Greiner bio-one) was homogenized in 9 ml chloroform/methanol (2/1, v/v) per tube using an Ultra-Turrax T25 homogenizer (IKA Labortechnik Staufen, Germany) for 3 min. Further homogenization was carried out for 2 min after adding 3 ml 1 M KCl to each tube. Each tube was centrifuged at 6,000 g for 3 min. The lower phase was transferred to a new tube (Tube B) and the solvent was evaporated under a flow of nitrogen at room temperature. The upper phase was mixed with 1 g of glass beads in a Vibramax mixer for 10 min and with vigorous vortexing for 1 min. 6 ml chloroform was added to each tube and mixed again for 3 min. After centrifugation, the lower phase was transferred to Tube B and the solvent was evaporated under a flow of nitrogen gas at room temperature. To extract remaining lipid, the upper phase in Tube A was mixed with another 6 ml chloroform and mixed for 3 min. After centrifugation, the lower phase was again transferred to Tube B. 3 ml methanol and 3 ml 0.1 M KCl were added to Tube B and mixed for 3 min. The lower phase was transferred to a Falcon tube and the solvent was evaporated under a flow nitrogen gas at room temperature. The extracted lipid was dissolved in chloroform/methanol (2/1, v/v) and stored at −20° C.

### Lipid Fractionation by Thin Layer Chromatography

To separate different lipid types such as TAG, DAG, free fatty acid and polar lipids such as phospholipids (PL), total lipids were fractionated on thin layer chromatography (TLC) plates (Silica gel 60; Catalog No. 1.05626.0001, MERCK, Darmstadt, Germany) using hexane: diethylether: acetic acid (70/30/1 v/v/v) as the solvent system. A sample of a lipid standard such as 18-6A containing TAG, DAG, FFA and MAG (Nu-Chek Prep Inc, USA) was run in an adjacent lane to identify the different lipid spots. When distinguishing different TAGs containing short-chain fatty acids (SCFA), a standard containing triheptadecanoin (Nu-chek, USA, Catalog No. T-155), a triglyceride mix C2-C10 containing equal amounts of triacetin (TAG 6:0), tributyrin (TAG 12:0), tricaprillin (TAG 18:0) and tridecanoin (TAG 30:0) (Sigma Aldrich, Catalog No 17810-1AMP-S) were run in adjacent lanes to identify the TAG lipid spots. After the chromatography, the plates were sprayed with a primuline (Catalog No. 206865, Sigma, Taufkirchen, Germany) solution prepared at a concentration of 5 mg/100 ml in acetone: water (80/20 v/v) and lipid bands visualised under UV light. The silica with the lipid from each spot was scraped off and transferred to a tube. The lipid fractions were extracted from the silica for derivatisation using either methylation, propylation or butylation.

Larger Scale Fractionation of PL and TAG from Total Lipid

PL and TAG were fractionated from about 100 mg of total lipid, extracted from approximately 10 g cell dry weight, by loading the lipid on 18 cm lines on each of eight TLC plates (Silica gel 60; Catalog No. 1.05626.0001, MERCK, Darmstadt, Germany) and chromatographed with a solvent mixture consisting of hexane/diethylether/acetic acid (70:30:1, v:v:v). An aliquot of a lipid standard containing TAG, DAG, FFA and MAG (18-6A; NuCheck Inc, USA) was run in parallel to assist with identifying the lipid bands. After staining the plates with primuline and visualisation under UV light, the PL bands located at the origin and the TAG bands having the same mobility as the TAG standard were collected and transferred to Falcon tubes. The lipid/silica samples were extracted with a mixture of 6 ml chloroform and 3 ml methanol, mixing vigorously for 5 min, then adding 3 ml water and further mixing for 5 min. After centrifugation for 5 min at 3,000 g, the lower organic phase was transferred to a new tube. The lower phase was transferred to a Falcon tube after centrifugation at 3000 rcf for 5 min. The upper phase was mixed with 5 ml chloroform for 5 min to extract any remaining lipid. After centrifugation, the lower phase was combined with the first extract. The solvent was evaporated under a flow of nitrogen gas. The extracted lipid, TAG or PL, was dissolved in a small volume of chloroform and filtered through 0.2 μm micro-spin filter (Chromservis, EU, Catalog No. CINY-02) to remove any particulates. The fatty acid composition and amount of each PL and TAG fraction were determined by preparation of FAME and GC analysis. Such preparations were used, for example, to separate different polar lipid classes such as PC, PE, PI and PS, or in Maillard reactions for aroma tests or for detection of volatile compounds as reaction products.

### Lipid Derivatisation to Fatty Acid Methyl Esters (FAME)

For analysis by GC, fatty acid methyl esters (FAME) were prepared from total extracted lipid or the purified TAG or PL fractions by treatment with 0.7 ml 1 N methanolic-HCl (Sigma Aldrich, Catalog No. 90964) in a 2 ml glass vial having a PTTE-lined screw cap at 80° C. for 2 h. A known amount of heptadecanoin (Nu-Chek Prep, Inc., Catalog No. N-7-A, Waterville, MN, USA) dissolved in toluene was added to each sample before the treatment as an internal standard for quantification. After the vials were cooled, 0.3 ml of 0.9% NaCl (w/v) and 0.1 ml hexane were added and the mixtures vortexed for 5 min. The mixture was centrifuged at 1700 g for 5 min and the upper, hexane phase containing the FAME was analysed by GC.

### Analysis and Quantification of FAME by GC

The individual FAMEs were identified and quantified by GC using an Agilent 7890A GC (Palo Alto, California, USA) with a 30 m SGE-BPX70 column (70% cyanopropyl polysilphenylene-siloxane, 0.25 mm inner diameter, 0.25 μm film thickness), a split/splitless injector and an Agilent Technologies 7693 Series auto sampler and injector, and a flame ionisation detector (FID). Samples were injected in split mode (50:1 ratio) at an oven temperature of 150° C. The column temperature was programmed for 150° C. for 1 min, increasing to 210° C. at 3° C./min, holding for 2 min and reaching 240° C. at 50° C./min, then holding at 240° C. for 0.4 min. The injector temperature was set at 240° C. and the detector at 280° C. Helium was used as the carrier gas at a constant flow of 1.0 ml/min. FAME peaks were identified based on retention times of FAME standards (GLC-411, GLC-674; NuChek Inc., USA). Peaks were integrated with Agilent Technologies ChemStation software (Rev B.04.03 (16), Palo Alto, California, USA) based on the response of the known amount of the external standard GLC-411 (Nucheck) and C17:0-ME internal standard. The resultant data provide the fatty acid composition on a weight basis, with percentages of each fatty acid (weight %) in a total fatty acid content of 100%. These percentages on a weight basis could readily be converted to percentages on a molar basis (mol %) based on the known molecular weight of each fatty acid.

### Saponification of Triacylglycerols

Free fatty acids were released from TAG by incubating 1 mg TAG in 0.2 ml 3M KOH for 3 min at 80° C. After cooling the sample to room temperature, 100 μl hexane was added to the mixture. The mixture was vortexed for 5 min, centrifuged at 1700 g for 5 min and the upper organic phase collected for GC analysis.

### Lipid Derivatisation to Ethyl Esters or Propyl Esters

To convert the fatty acids in TAG to fatty acid ethyl esters (FAEE), 2 mg of TAG was incubated in 1N HCl/ethanol solution at 80° C. for 2 h. After cooling the sample to room temperature, 100 μl hexane was added to the mixture. The mixture was vortexed for 5 min, centrifuged at 1700 g for 5 min and the upper organic phase collected for GC analysis. To convert the fatty acids in TAG to fatty acid propyl esters, 2 mg of TAG was incubated in 1N HCl/propanol rather than 1N HCl/ethanol and otherwise processed the same.

### Derivatisation of Fatty Acids in TAG to Butyl Esters

TAG fractions were extracted from the silica of the TAG spots on TLC plates as follows: 0.6 ml chloroform:methanol (2:1, v/v) was added to silica scraped from the TLC plate. The mixture was shaken and centrifuged for 5 min at 10,000 g. Then, 0.3 ml of 0.1M KCl was added and the mixture shaken for 5 min. The mixture was centrifuged for 5 min at 10,000 g and the lower, organic phase collected in a 2 ml GC vial. The silica/aqueous phase was extracted a second time, this time with 0.3 ml chloroform, mixing for 10 min followed by centrifugation at 10,000 g for 5 min. The lower, organic phase was again collected and pooled into the same GC vial as the first extract. The pooled extract containing the TAG was filtered through a 0.2 μm micro-spin filter (Chromservis, EU, Catalog No. CINY-02) to remove traces of silica particles. The filtered TAG extract was then transferred into GC vial with flat insert and completely dried under a stream of nitrogen.

The purified TAG was then derivatised to butyl esters using 60 μl of butanolic:1N HCl (Sigma Aldrich, Catalog No. 87472) as described by Mannion et al. (2018), with some modifications. Valeric acid (C5:0) (Sigma Aldrich, Catalog No. 75054) was added as internal standard at an amount of 23.25 μg for SCFA and MCFA quantification and 5 μg of heptanoic acid (Nu-Chek PREP, Inc., Catalog No. N-7-A Waterville, MN, USA) as internal standard for LCFA quantification. The mixture was vortexed and heated for 2 h at 80° C. The reaction was then stopped by adding 0.03 ml of water and 0.03 ml of hexane, and thoroughly mixed for 10 min. After centrifugation at 1700 g for 5 min, the upper, organic phase was transferred into a new tube with flat insert containing 0.1 ml of saturated NaCl for a second wash to remove traces of butanol. The mixture was mixed for 5 min, centrifuged at 1700 g for 5 min and the organic phase transferred into a new GC vial with conical insert, capped quickly for GC-FID analysis as described below.

### Analysis and Quantification of Butyl Esters

This method was suitable for the quantitation of short chain fatty acids (SCFA, C2-C8) as well as medium (MCFA, C10-C14) and long chain fatty acids (LCFA, C16-C18) in lipid samples, including in purified TAG preparations. It was the preferred method for quantitation of SCFA. FABEs prepared as described above were analysed on an Agilent 7890A GC using a 30 m BPX70 Column (0.25-mm inner diameter, 0.25-μm film thickness, SGE, Australia). The column temperature was set for 1 min at 40° C., followed by raising the temperature at a rate of 3° C./min to 210° C., which was held for 2 min. The column temperature was further raised to 240° C. at a rate of 100° C./min and held at this temperature for 0.5 min. Helium was used as a carrier gas at a flow rate of 1.031 ml/min. The injector temperature was programmed at 240° C. with 11.8 psi inlet pressure. The samples were injected in the split mode with a ratio of 50:1. The FID detector temperature was 280° C. with a flow of 40 ml/min hydrogen gas, 400 ml/min of air and 25 ml/min make-up gas (He). FABE peaks were identified based on retention times of FABE standard mix prepared with equal amounts of analytical grade C4-C18:1 fatty acids. Peak areas of the FABE mix were used to determine the response factors for individual FABE peaks in the GC and were applied to correct the area percentages of the FABE peaks.

In a variation of the GC method for quantitation of FABE, referred to herein as the “C8C24 method”, some column parameters were adjusted. The column temperature was set for 13 min at 40° C., followed by raising the temperature at a rate of 320° C./min to 210° C., which was held for 2 min. The column temperature was further raised to 240° C. at a rate of 10° C./min and held at this temperature for 0.53 min. The injector temperature, FID detector temperature and helium flow were as before.

### Peak Identity by GC-MS

The identities of unknown or uncertain peaks in the GC-FID chromatograms were confirmed by Gas Chromatography Mass Spectrometry (GC-MS) analysis. Samples were run on a GC-MS operating in the Electron Ionization mode at 70 eV to confirm peak identities and to identify possible extra peaks corresponding to possible contamination, degradation products or reagent signals. A Shimadzu GC-MS QP2010 Plus (Shimadzu Corporation, Japan) system coupled to an HTX-Pal liquid auto-sampler was used with the following parameters: 1 or 2 μl injection volume using a split/splitless inlet at a 15:1 split, at a temperature of 250° C. The oven temperature program used was the same as for the GC-FID. MS ion source and interface temperatures were 200° C. and 250° C., respectively. Data were collected at a scan speed of 1000 and scan range from 40 to 500 m/z. Peak separation was provided by a Stabilwax or Stabilwax-DA (Restek/Shimadzu) capillary column (30 m×0.25 mm i.d., 0.25 μm film thickness) using He as a carrier gas at 30 cm/sec. Mass spectra correlations were performed using a NIST library, retention indices and matching retention time of available standards. Identified SCFA was set to be present when S/N ratio were above 10:1. Instrument blanks and procedural blanks were run for quality control purposes.

### Analysis of Volatile Compounds by Solid-Phase Micro-Extraction Gas Chromatography Mass Spectrometry (HS-SPME-GCMS).

### Reagents and Chemical Standards

Analytical standards were obtained from Sigma Aldrich (USA) and represented different chemical classes of compounds in Maillard reaction: 1-octen-3-ol, methional, 2 (5H)-furanone, 2-methyl-3-heptanone, 1-pentanol, pentanal, hexanal, nonanal, 1-heptanal, octanal, trans-2-nonenal, isovaleric acid, 2-pentyl-furan, 2,4,6-trimethyl-pyridine. Standard stock and working solutions were prepared in methanol (LCMS grade). A series of n-alkanes (C8-C20) mixture was purchased from Supelco (USA) and diluted in hexane for injection in the GC-MS.

### Headspace HS-SPME-GCMS Method

HS-SPME was performed using a 50/30 μm divinylbenzene-carboxen-polydimethylsiloxane (DVB/CAR/PDMS) Stableflex fiber (Supelco, USA), 10 mm long, for automatic autosamplers. Samples with varied amounts (0.5, 1, 2 and 7.4 ml) were used to test the best volume needed for the analysis. Samples were conditioned for 10 min at 50° C. in a 10 ml headspace magnetic cap vial prior to extraction. Volatiles were extracted for 20 min at 50° C. under agitation using a Combi-Pal autosampler HTX PAL (CTC Analytics). Samples were desorbed at the injector temperature of 240° C. for 1 min at splitless mode. Each fibre was conditioned in a needle heater with a helium flow at 240° C. for 8 min before and after sample desorption to reduce carryover from a previous sample. The volatile compounds desorbed from the fibre were analysed by a Shimadzu QP2010 Plus GC-MS equipped with a Restek Stabilwax column (30 m×0.25 mm×0.25 μm). The carrier gas was helium at a constant flow rate of 1 ml/min. Oven ramping program started at 40° C. held for 3 min, heated to 240° C. at a rate of 4° C. per min and held for 2 min. Ion fragmentation was acquired under EI mode at 70 eV and scanned in full scan mode from 40 to 400 m/z. Volatiles were identified by comparing NIST mass spectra library and linear retention indexes calculated using a series of n-alkanes (C8-C20) as external references. Purchased authentic standards from different compound classes and blanks (empty HS vials) were also analysed for analytical quality control. Mass spectra matches were only considered with a minimum of 80% similarity index. Peaks were selected for identification of volatile compounds such as Maillard reaction compounds at a S/N ratio of 2.

### Recombinant DNA Methods

Derivatives of pYES2 having single genes inserted for testing in yeast were made by inserting protein coding regions between the unique HindIII and XhoI sites or other restriction enzyme sites in the plasmid as appropriate by standard cloning methods. The E. coli strain DH5α was used for cloning and plasmid propagation and DNA preparation according to standard methods.

The GoldenGate (GG) method (Larroude et al., 2018) allows for rapid and efficient combinatorial assembly of multiple expression cassettes in a single vector and was therefore used to make multigene constructs for testing in S. cerevisiae or Y. lipolytica. GG DNA parts and donor vectors, also called L0 vectors, according to Celinska et al. (2017) and Larroude et al. (2018) were obtained from Addgene, USA. The DNA parts included promoters (GGE146, GGE151 and GGE294), terminators (GGE014, GGE015, GGE080, GGE020 and GGE021) and the backbone assembly vector (destination vector) was GGE114.

Protein coding regions for insertion into the vectors by GG assembly were codon optimised for S. cerevisiae or Y. lipolytica using Twist Bioscience and GeneArt online software (Twist Bioscience: www.twistbioscience.com/products/genes; ThermoFisher/GeneArt: www.thermofisher.com/au/en/home/life-science/cloning/gene-synthesis/geneart-gene-synthesis.html) and synthesised either by Twist Bioscience or GeneArt (ThermoFisher, USA), or in the lab. Internal BsaI restriction enzyme sites were avoided in the codon optimised nucleotide sequences of the protein coding regions as BsaI sites were used in the GG assembly method. NotI restriction enzyme sites were also avoided within the nucleotide sequences as NotI was used for linearizing the genetic constructs for transformation of Y. lipolytica. When one, two or three genes were to be inserted into a single vector, the individual components were designed with 4-nucleotide overhangs immediately 5′ of each translation start codon (ATG) and 3′ of the translation stop codon, with the sequence of each 4-nucleotide overhang depending on the position of the component in the backbone vector GGE114, according to Table 3. The external BsaI site with the appropriate 4-nt overhang was added to the 5′ end of each DNA strand.

The protein coding regions were synthesised in a cloning vector having a kanamycin selection marker gene to avoid any false positives when performing the GG reaction with the GG backbone vector GGE114 which had an ampicillin selectable marker gene. The E. coli strain DH5α was used for cloning and plasmid propagation according to standard methods. Antibiotics were used as appropriate for selecting transformed cells, for example ampicillin was added at 100 g/mL for selection of constructs having an ampicillin selectable marker gene.

The destination vector GGE114 contained the red fluorescence protein (RFP) chromophore, which acts as a colour-based visual marker for negative cloning in E. coli, as described by Larroude et al. (2018). The vector GGE114 was a preassembled destination vector that, in addition to the bacterial replicon, contained popular bricks ZETA sequences in the place of InsUp and InsDown fragments and the URA3 marker with a view to reducing the number of fragments to assemble when employing this combination, into the backbone vector pYES2 which contained a 2μ origin for high-copy maintenance. In this case, the RFP was between the URA3 marker and the ZETA down. In the presence of BsaI enzyme, the RFP was released and the one, two or three transcription units (TU; promoter-protein coding region-terminator) were inserted.

The GG assembly reaction mixes contained equimolar quantities (50 ng) of the GG backbone vector such as GGE114 and other DNA components (donor vectors) in a final volume of 7.5 μl, by adding 0.75 μl 10×T4 ligase buffer, 0.75 μl 10×BSA (bovine serum albumin), 0.75 μl BsaI HF-V2 (NEB), 0.5 μl T4 ligase (NEB). The reaction mixtures were incubated with 25 cycles of 37° C. for 3 min followed by 16° C. for 4 min, then 1 cycle of 50° C. for 5 min and 80° C. for 5 min. Samples of 2-3 μl were introduced into competent cells of E. coli strain DH5α by standard methods. Colonies lacking the RFP were confirmed to contain the desired genetic inserts by colony PCR with the appropriate primers and verified with restriction digests. Glycerol stocks were made and stored at −80° C.

TABLE 3
Nucleotide sequences of overhangs used in
GoldenGate cloning method

| | | |
|---|---|---|
| Position | 5′ overhang | 3′ overhang |

One gene insertion

| | | |
|---|---|---|
| Promoter 1 | ACGG | AATG |
| Gene 1 | AATG | TCTA |
| Terminator 1 | TCTA | GAGT |

Two gene insertion

| | | |
|---|---|---|
| Promoter 1 | ACGG | AATG |
| Gene 1 | AATG | TCTA |
| Terminator 1 | TCTA | GCTT |
| Promoter 2 | GCTT | ACAA |
| Gene 2 | ACAA | GGAT |
| Terminator 2 | GGAT | GAGT |

Three gene insertion

| | | |
|---|---|---|
| Promoter 1 | ACGG | AATG |
| Gene 1 | AATG | TCTA |
| Terminator 1 | TCTA | GCTT |
| Promoter 2 | GCTT | ACAA |
| Gene 2 | ACAA | GGAT |
| Terminator 2 | GGAT | GTCA |
| Promoter 3 | GTCA | CCAC |
| Gene 3 | CCAC | GTAT |
| Terminator 3 | GTAT | GAGT |

Transformation of S. cerevisiae

A rapid method was used for introduction into S. cerevisiae of genetic constructs based on pYES2 which did not use competent cells. A loop full of S. cerevisiae cells was scraped off a fresh plate and the cells resuspended in 100 μl of transformation buffer (Sigma Aldrich, Catalog No. T0809). About 1 μg of plasmid DNA with 10 μl of 10 mg/ml salmon testes DNA which had been boiled for 5 min prior to use were added to the cell suspension along with 600 μl of plate buffer (Sigma Aldrich, Catalog No. P8966) and mixed well. The mixture was incubated at room temperature in a rotor wheel at the lowest speed for 16 hours. The mixture was then heat shocked for 15 min at 42° C., spun at 3500 rpm for 3 min, and the pellet of cells resuspended in 200 μl of sterile water. Aliquots of up to 100 μl were plated out onto synthetic drop-out selection media lacking uracil (SD-URA, Sigma Aldrich, Catalog No. Y1501) for selection of transformants. The plates were incubated at 28° C. for 3 days or until colonies appeared. Two or more colonies were picked from each plate and tested for the presence of the genetic construct by colony PCR to identify transformants.

Transformation of Y. lipolytica for Integration of Expression Cassettes

DNA of genetic constructs which included the expression cassettes (transcription units) for insertion into Y. lipolytica by homologous recombination was digested with NotI or other appropriate restriction enzymes to release the expression cassette. The linearised DNA was introduced into competent cells of the selected Ura-Y. lipolytica strain, prepared using the Frozen-EZ Yeast Transformation II kit (Zymo Research, California, USA). Briefly, 5 μl (2 μg) of the NotI digested and linearised expression vector was mixed with 50 μl competent cells and 500 μl of EZ3 solution from the kit and mixed thoroughly. A negative control transformation included competent cells without any DNA of the genetic construct. The mixtures were incubated at 28° C. for two hours and then 100 μl spread on a SD-Ura plate. The plates were incubated for two days at 28° C. When the recipient strain was an auxotroph lacking a functional URA3 gene, only transformants having received the vector with the URA gene grew on these plates. Many colonies were observed in the Y. lipolytica transformations. Ura⁺ colonies were picked from the selection plates and confirmed as transformed by colony PCR for the introduced genetic construct and the phenotype corresponding to intended genetic modification.

### Gene Expression Analysis

Expression of transgenes was analysed using a DNase RQ1 kit (Promega Catalog No. M6101) and a Qiagen column (Qiagen RNAse-free DNAse) to purify RNA from the cells, and oligo dT primer (200-500 ng), dNTPs (10 mM), Superscript III reverse transcriptase and 0.1 M DTT for reverse transcription using standard methods.

### Example 2. Polar Lipid Content and Composition of Animal Fats

The flavour and aroma of cooked animal products such as meats comes from a wide variety of compounds including peptides, amino acids, sugars, vitamins such as thiamine, and lipids including phospholipids (Dashdorj et al., 2015; Resconi et al., 2013). The main sources of volatiles in cooked meat are from the Maillard reaction between amino acids and sugars and from the thermal degradation of lipids. There are published reports of the content and composition of phospholipids in animal products, for example Ashes et al. (1992), Margetak et al. (2012) and Resconi et al. (2013), but many of these reports relate more generally to polar lipids or phospholipids, or are only partial for fatty acid composition and do not report on the presence of minor fatty acids such as fatty acids with an odd number of carbons, conjugated fatty acids such as CLA, or trans fatty acids which are reported to be common in animal-sourced food products.

The present inventors investigated the fatty acid composition of the phospholipid fraction from several animal sources including beef and pork, in a more thorough manner.

Extraction of Lipids from Animal Sources

Pork and wagyu beef samples were purchased at a local market in Canberra, Australia. To extract lipid from the meat, 2 g samples of minced meat were homogenized in 6 ml solvent of chloroform/methanol (2/1, v/v) for 3 min with an Ultra-Turrax homogenizer (IKA Labortechnik Staufen, Germany) in a 50 ml conical-bottom polypropylene tube (Falcon tube, No. 227280; Greiner bio-one GmbH, Germany) (Tube A). 2 ml of 1 M KCl was added to the homogenate, and the mixture was further homogenized for 3 min followed by mixing for 10 min in a vibramax. After centrifugation for 5 min at 3,000 g, the lower organic phase was transferred to a new Falcon tube (Tube B). The extraction was repeated with 4 ml chloroform, homogenization and centrifugation as before. The lower organic phase was collected and added to Tube B. The solvent was evaporated from Tube B under a flow of nitrogen at room temperature. The extraction was again repeated with 4 ml chloroform and the lower phase was transferred to the Tube B. Two ml each of methanol and 0.1 M KCl were added to the Tube B, mixed for 3 min and centrifuged. The lower phase was transferred to a new tube, Tube C. The upper phase was mixed with 2 ml fresh chloroform, centrifuged, and the lower phase again transferred to Tube C. The solvent was evaporated from Tube C with nitrogen. The extracted lipid was dissolved in chloroform, transferred to a glass vial and stored at −20° C. A sample of the extracted lipid was set aside for analysis as “total lipid”.

A lipid product sold commercially as lard (York Foods) was purchased and analysed.

### Lipid Fractionation and Determination of Fatty Acid Composition

To separate different lipid types such as TAG, DAG, free fatty acid and polar lipids including the phospholipids (PL), total lipids extracted from the pork, wagyu beef and lard were fractionated on TLC plates and recovered as described in Example 1. Lipid fractions were extracted from the silica spots, converted to FAME, then analysed and quantitated by GC as described in Example 1.

### Fractionation of Polar Lipids to Separate PL Classes

To investigate the composition of PL classes in extracted polar lipids, for example to determine the ratios of different PL amounts or their fatty acid composition, polar lipid extracts from the beef, pork and lard were applied to the origin of a TLC plate (Silica gel 60; Catalog No. 1.05626.0001, MERCK, Darmstadt, Germany) and chromatographed using a solvent mixture of chloroform/methanol/acetic acid/water (90:15:10:3, v:v:v: v). Lipid bands were visualized by spraying the plates with 0.002% (w/v) primuline solution in 80% acetone/water (v/v) and viewing under UV light. The phospholipid classes were identified by reference to known phospholipid standards applied to adjacent lanes on the TLC plate. The phospholipid standards, namely PC (Cat. No. 850375), PE (Cat. No. 850725), PS (Cat. No. 840035), PI (Cat. No. 850149), PG (Cat. No. 840475), PA (Cat. No. 840875) and LPC (Cat. No. 845875) were purchased from Avanti Polar lipids (USA) and cardiolipin from Sigma (Cat. No. 1649). Separation was achieved for all of these phospholipid classes on the TLC plates. This procedure also separated any galactolipids from the PLs. The silica containing the individual bands were collected into glass vials for further characterization of the lipid classes, including their relative amounts, fatty acid composition and fatty acid distribution in the sn-1 and sn-2 positions of the PL molecules.

Fatty acid methyl esters (FAME) were prepared from total extracted lipid or the purified TAG or polar lipid fractions and analysed by GC as described in Example 1. The peak areas were integrated with Agilent Technologies ChemStation software (Rev B.04.03 (16)) and the lipid content and fatty acid composition in each sample were calculated on the basis of the area of the internal standard (heptadecanoin).

### Confirmation of the Identity of Peaks by GC-MS

The identity of some of the FAME peaks was confirmed by GC-MS, in particular where the identity of a peak was not clear from the retention time in the GC chromatogram, such as for minor or uncommon fatty acids and to identify possible extra peaks corresponding to solvents, degradation products or reagent signals. The analysis was performed as described in Example 1.

Fatty acid composition of TAG and polar lipid fractions from pork, beef and lard

The fatty acid composition of the total fatty acid (TFA) content of the extracted lipid and the TAG and polar lipid fractions were determined for the pork, and wagyu beef and lard samples by GC quantitation of FAME as described in Example 1. The fatty acid composition data are presented in Table 4, expressed for each fatty acid as a percentage of the total fatty acid content on a weight basis. The standard deviations were generally 0.1 or less.

As expected for animal lipids, all of the fractions were high in the percentages for saturated fatty acids (SFA), including about 17% to 28% palmitic acid and about 12% to 18% stearic acid. Monounsaturated fatty acids (MUFA) were also present at substantial amounts including oleic acid at about 40% in the TAG and about 18% in the polar lipid. Palmitoleic acid (C16:1Δ9) was also present in all fractions at a lower amount, at about 1-3%. The other monounsaturated fatty acids present were C16:1Δ7, C17:1Δ9, C18:1Δ11 and the unusual fatty acid C20:1Δ8. Polyunsaturated fatty acids (PUFA) of both the ω3 and ω6 classes were also present in all fractions, such as ALA, EPA and DPA for the ω3 fatty acids and DGLA, ARA and DTA for the ω6 fatty acids. Several minor fatty acids were also noted, including the odd-chain fatty acids C15:0 and C17:0, and a minor peak which had a retention time approximating that of conjugated linoleic acid (CLA), but not confirmed by GC-MS. Meat is known to contain both branched chain and straight chain fatty acids having an odd number of carbons (Taormina et al., 2020). A minor peak having a retention time close to that of C15:1 was shown by GC-MS to be decanal-dimethylacetal, which is not a fatty acid. Similarly, minor peaks having a retention time close to that of C17:1t were demonstrated by GC-MS to be related to decanal-dimethylacetal and 1,1-demethoxy-dodecane. Those peaks were therefore excluded from the quantitation. The presence of both trans fatty acids and various CLA, in particular the cis-9, trans-11 CLA isomer also referred to as rumenic acid, is characteristic of animal polar lipids (Aro et al., 1998; Daley et al., 2010; Palmquist et al., 2005).

Several differences were noted for the fatty acid composition of animal polar lipids relative to TAG. The total amount of saturated fatty acids was lower in the polar lipids at just below 30% for the pork and beef, whereas the total amount of polyunsaturated fatty acids (PUFA) was higher in each polar lipid fraction, particularly for the ω6 fatty acids which was predominantly LA. The medium chain, saturated fatty acids C10:0 and C12:0 were present at low levels in the TAG fractions from pork and beef but not in the polar lipid fractions. It was noted, most significantly to the inventors, that the levels of ARA, DGLA and DTA were at least 10-fold higher in the polar lipid fractions than the corresponding TAG fractions, for example with ARA at about 8% in polar lipids but only at 0.2% in TAG, indicating that cattle and pigs do not have an efficient means for transferring the C20 ω6 fatty acids from polar lipids where they are synthesized to the storage lipid, TAG.

The fatty acid compositions determined for beef and pork were similar in the classes of major fatty acids as reported by Bermingham et al. (2018), Daley et al. (2010), Farmer et al. (1990), Dannenberger et al. (2006), Hornstein et al. (1961), Meynier et al. (1998), Melton (1999) and Wood et al. (2003, 2008), although there was considerable variation between those references in the precise percentages and those reports generally did not analyse all fatty acids for the full fatty acid profile. In each case where they were analysed for ω3 fatty acids, the animal polar lipids contained ALA, DPA and DHA as well as, in many cases, EPA. Farmer et al. (1990) also measured the fatty acid composition of egg phosphatidylcholine (PC) and phosphatidylethanolamine (PE) and reported the presence of ω3 fatty acids, odd chain fatty acids C15:0 and C17:0, and substantial levels of ARA in these phospholipids. Saturated fatty acids were again high at 44-46%, predominantly composed of C16:0 and C18:0.

Larger Scale Purification of PL from Meat and Fractionation into Phospholipid Classes

In order to analyse larger scale preparations of polar lipids from meat and fractionate the polar lipid into its constituent phospholipid classes, lipid was extracted again as described above. To separate the neutral (non-polar) lipids including TAG, DAG and free fatty acids (FFA) from polar lipids, the extracted lipid was fractionated by loading the lipid on 18 cm lines on 8×TLC plates (Silica gel 60; Merck) and chromatographed with a solvent mixture of hexane/diethylether/acetic acid (70/30/1, v/v/v). The silica containing the polar lipid located at the origin on each plate was collected and transferred to a Falcon tube. Similarly, the silica containing each TAG band, running at the level of a TAG standard, was collected into a tube. The lipid/silica samples were mixed with 6 ml chloroform/methanol for 5 min, then 2 ml MilliQ water added and mixed again for 5 min. The lower phase was transferred to a Falcon tube after centrifugation at 3,000 g for 5 min. The upper phase was mixed with 4 ml chloroform and extracted again for 5 min. After another centrifugation, the lower phase was transferred to the tube containing the first extract and the solvent was evaporated under a flow of nitrogen. The dried lipid was dissolved in a small volume of chloroform and filtered through 0.2 μm micro-spin filter (Chromservis, EU, Catalog No. CINY-02) to remove particulates. The fatty acid compositions and amounts of polar lipid and TAG were determined by preparing FAME from the TAG and polar lipid aliquots, using a known amount of heptadecanoin as internal standard, and GC analysis of the FAME as described in Example 1.

### Analysis of PL Classes of Meat

To separate the different phospholipid (PL) classes and determine their amounts and fatty acid composition, the polar lipids from beef and pork were fractionated by TLC (Silica gel 60, Merck) using a solvent mixture of chloroform/methanol/acetic acid/water (90/15/10/3, v/v/v/v). The phospholipid spots were identified by reference to phospholipid standards (see above) run in adjacent lanes. Separation was achieved for all of these phospholipid classes on the TLC plates. This procedure also separated the sphingolipids and any galactolipids from the PLs. The silica containing the individual bands were collected into glass vials, mixed with known amounts of triheptadecanoin and converted to FAME and quantitated by GC as described in Example 1.

The data for the fatty acid composition for phospholipids from pork meat is shown in Table 5 and for beef in Table 6. Abbreviations: PC, phosphatidylcholine; PE, phosphatidylethanolamine; PI, phosphatidylinositol; PS, phosphatidylserine; PA, phosphatidic acid; PG, phosphatidylglycerol; LPC, lysophosphatidylcholine. PC was the most abundant phospholipid (53.3% of the polar lipids) in pork meat, followed by PE (35.5%), with much lower amounts of PI (4.0%). The relative amounts were roughly similar to the results reported by Boselli et al. (2008) and Meynier et al. (1998). PS, PG, PA and LPC constituted minor proportions of PL at between 0.5% and 1.6% each. A higher proportion of ARA was observed in PE (13.4%) and PI (8.3%), while PC contained 3.2% ARA. Also, PE was richer in DGLA, 22:4n6, and 18:0 levels, while PC showed higher levels of 16:0, 18:1. PI and PS demonstrated higher level of 18:0.

The extracted lipid preparations described above are also analysed by GC of fatty acid butyl esters (FABE), prepared as described in Example 1. The amount of short chain fatty acids (C4, C6 and C8) in the lipid preparations is established. The fatty acid distribution in the sn-1 and sn-2 positions of the phospholipids is also determined. Animal phospholipids are reported to have mainly saturated fatty acids at the sn-1 position and unsaturated fatty acids such as oleic, linoleic, linolenic and arachidonic acids at the sn-2 position (Kullenberg et al., 2012; Rong et al., 2015).

### Example 3. Extraction of Lipids from Microbes

Lipid extraction from yeasts such as S. cerevisiae and Y. lipolytica is made more difficult by the rigid cell wall of these organisms. Various methods have been described in the literature for cell disruption and lipid extraction from yeasts, including mechanical, enzymatic, chemical, osmotic shock and microwave methods of cell disruption (Hein and Hayen, 2012), Chisti and Moo-Young (1986) reviewed mechanical methods of microbial cell disruption as well as cell lysis by osmotic shock, and chemical and enzymatic methods. Hegel et al. (2011) described lipid extraction from yeast using supercritical carbon dioxide. Peter et al. (2017) reported cell disruption and homogenization of Schizosaccharomyces pombe cells in a water/methanol solvent mixture using zirconium oxide beads, a bullet blender and a water bath sonicator. A simple, high throughput and small-scale lipid extraction method was desirable to perform the studies described in the following Examples. The inventors therefore tested several methods and variations for the extraction of lipids from S. cerevisiae and Y. lipolytica, in particular tested some methods for cell wall disruption and homogenisation of the cellular material with organic solvents to extract the lipid and testing the efficiency of extraction.

TABLE 4
Fatty acid composition of total lipid (TFA), triacylglycerol
and polar lipid fractions extracted from waygu beef and
pork meat, by GC quantitation of FAME. Figures for each
fatty acid are the percentage of the total fatty acid
content, except for the last row (mg/100 mg meat).

| | Beef | | | Pork | | | Lard | |
|---|---|---|---|---|---|---|---|---|
| Fatty acid | TFA | TAG | Polar | TFA | TAG | Polar | TAG | Polar |
| C10:0 | 0.1 | 0.1 | 0.0 | 0.1 | 0.1 | 0.0 | 0.1 | 0.0 |
| C12:0 | 0.2 | 0.2 | 0.0 | 0.2 | 0.2 | 0.0 | 0.2 | 0.9 |
| C14:0 | 1.8 | 1.9 | 0.4 | 2.0 | 2.1 | 0.2 | 1.9 | 1.2 |
| (myristic) | | | | | | | | |
| C15:0 | 0.1 | 0.1 | 0.2 | 0.1 | 0.1 | 0.4 | 0.1 | 3.8 |
| C16:0 | 27.0 | 27.0 | 18.2 | 26.9 | 27.5 | 17.5 | 28.2 | 19.3 |
| (palmitic) | | | | | | | | |
| C16:1Δ7 | 0.4 | 0.4 | 0.5 | 0.4 | 0.4 | 0.6 | 0.0 | 0.0 |
| C16:1Δ9 | 3.1 | 3.2 | 1.4 | 2.6 | 2.6 | 1.5 | 1.9 | 0.9 |
| C17:0 | 0.4 | 0.3 | 0.6 | 0.4 | 0.4 | 0.6 | 0.0 | 0.0 |
| C17:1Δ9 | 0.3 | 0.4 | 0.4 | 0.4 | 0.4 | 0.4 | 0.3 | 0.0 |
| C18:0 | 4.5 | 13.9 | 14.3 | 14.8 | 15.4 | 12.5 | 18.0 | 14.3 |
| (stearic) | | | | | | | | |
| C18:1 | 42.0 | 42.1 | 18.1 | 39.7 | 39.4 | 19.2 | 33.3 | 31.2 |
| (oleic) | | | | | | | | |
| C18:1Δ11 | 0.0 | 4.1 | 3.9 | 3.4 | 3.3 | 4.0 | 2.3 | 1.2 |
| C18:2 (LA) | 6.9 | 4.3 | 25.7 | 6.6 | 5.9 | 27.3 | 10.3 | 12.5 |
| C18:3ω6 | 0.0 | 0.0 | 0.5 | 0.0 | 0.0 | 0.5 | 0.1 | 1.3 |
| (GLA) | | | | | | | | |
| C18:3ω3 | 0.3 | 0.3 | 0.7 | 0.4 | 0.4 | 0.7 | 1.0 | 0.4 |
| (ALA) | | | | | | | | |
| C20:0 | 0.2 | 0.2 | 0.4 | 0.2 | 0.2 | 0.2 | 0.2 | 0.9 |
| C20:1Δ8 | 0.7 | 0.7 | 0.2 | 0.6 | 0.7 | 0.2 | 0.0 | 0.0 |
| C20:2ω6 | 0.2 | 0.2 | 0.2 | 0.2 | 0.2 | 0.2 | 0.3 | 0.0 |
| (EDA) | | | | | | | | |
| C20:3ω6 | 0.2 | 0.1 | 1.2 | 0.1 | 0.1 | 1.2 | 0.1 | 0.5 |
| (DGLA) | | | | | | | | |
| C20:4ω6 | 1.0 | 0.2 | 8.1 | 0.4 | 0.2 | 8.6 | 0.2 | 3.1 |
| (ARA) | | | | | | | | |
| C20:3ω3 | 0.0 | 0.1 | 0.1 | 0.1 | 0.1 | 0.1 | 0.1 | 0.0 |
| (ETrA) | | | | | | | | |
| C22:0 | 0.0 | 0.0 | 0.5 | 0.0 | 0.0 | 0.4 | 0.0 | 1.5 |
| C22:1 | 0.0 | 0.0 | 0.5 | 0.0 | 0.0 | 0.1 | 0.0 | 0.0 |
| C20:5ω3 | 0.0 | 0.0 | 0.5 | 0.0 | 0.0 | 0.4 | 0.0 | 0.0 |
| (EPA) | | | | | | | | |
| C22:4ω6 | 0.2 | 0.1 | 1.2 | 0.1 | 0.1 | 1.2 | 0.1 | 0.0 |
| (DTA) | | | | | | | | |
| C24:0 | 0.0 | 0.0 | 0.6 | 0.0 | 0.0 | 0.5 | 0.0 | 0.0 |
| C24:1 | 0.0 | 0.0 | 0.6 | 0.0 | 0.0 | 0.5 | 0.0 | 0.0 |
| C22:5ω3 | 0.2 | 0.1 | 0.9 | 0.1 | 0.1 | 0.9 | 0.0 | 0.0 |
| (DPA) | | | | | | | | |
| C22:6ω3 | 0.1 | 0.0 | 0.4 | 0.1 | 0.0 | 0.4 | 0.1 | 0.0 |
| (DHA) | | | | | | | | |
| Total SFA | 43.6 | 43.5 | 30.0 | 44.4 | 45.7 | 27.4 | 48.7 | 41.9 |
| Total MUFA | 45.7 | 50.7 | 21.8 | 46.7 | 46.6 | 22.5 | 37.8 | 33.3 |
| Total ω6 | 8.4 | 4.9 | 31.5 | 7.3 | 6.5 | 33.1 | 11.1 | 17.4 |
| PUFA | | | | | | | | |
| Total ω3 | 0.6 | 0.5 | 2.2 | 0.7 | 0.6 | 2.1 | 1.2 | 0.4 |
| PUFA | | | | | | | | |
| mg/100 | 13.7 | 5.6 | 0.7 | 37.2 | 14.8 | 0.4 | 14.5 | 0.03 |
| mg meat | | | | | | | | |

TABLE 5
Fatty acid composition and proportions of individual phospholipid classes
in the polar lipid of pork meat. Figures for each fatty acid are the
percentage of the total fatty acid content of the lipid class.

| | | | | | | | | Total |
|---|---|---|---|---|---|---|---|---|
| Fatty acid | PC | PE | PI | PS | LPC | PA | PG | polar |
| C10:0 | | | | | | | | 0.0 |
| C12:0 | 0.0 | 0.1 | 0.2 | 0.3 | 0.6 | 0.5 | 1.3 | 0.0 |
| C14:0 | 0.3 | 0.1 | 0.2 | 0.3 | 1.0 | 0.7 | 0.8 | 0.2 |
| C15:0 | 0.3 | 0.0 | 0.0 | 0.2 | 0.0 | 1.9 | 0.0 | 0.4 |
| C16:0 | 27.9 | 3.8 | 12.4 | 6.8 | 41.0 | 25.1 | 33.5 | 17.5 |
| C16:1Δ7 | 0.7 | 0.0 | 0.1 | 0.0 | 1.0 | 0.7 | 0.5 | 0.6 |
| C16:1Δ9 | 1.6 | 1.1 | 0.2 | 0.4 | 0.8 | 2.6 | 0.9 | 1.5 |
| C17:0 | | | | | | | | 0.6 |
| C17:1Δ9 | 0.5 | 0.3 | 0.1 | 0.4 | 0.5 | 0.5 | 0.7 | 0.4 |
| C18:0 | 7.5 | 15.6 | 49.1 | 50.2 | 30.8 | 31.4 | 41.6 | 12.5 |
| (stearate) | | | | | | | | |
| C18:1Δ9 | 25.1 | 10.3 | 3.9 | 12.8 | 11.2 | 11.8 | 5.7 | 19.2 |
| (oleate) | | | | | | | | |
| C18:1Δ11 | 4.1 | 3.4 | 2.1 | 1.8 | 1.9 | 2.1 | 2.5 | 4.0 |
| C18:2 (LA) | 25.1 | 36.5 | 3.3 | 10.7 | 5.0 | 14.7 | 5.9 | 27.3 |
| C18:3ω6 | 0.5 | 0.5 | 0.4 | 0.6 | 0.4 | 0.6 | 1.0 | 0.5 |
| (GLA) | | | | | | | | |
| C18:3ω3 | 0.7 | 0.9 | 0.2 | 0.3 | 0.4 | 0.8 | 0.0 | 0.7 |
| (ALA) | | | | | | | | |
| C20:0 | 0.0 | 0.1 | 2.9 | 1.5 | 0.5 | 0.3 | 0.0 | 0.2 |
| C20:1Δ11 | 0.2 | 0.4 | 0.2 | 1.1 | 0.0 | 0.0 | 0.0 | 0.2 |
| C20:2ω6 | 0.2 | 0.3 | 0.0 | 0.2 | 0.4 | 0.0 | 0.0 | 0.2 |
| (EDA) | | | | | | | | |
| C20:3ω6 | 0.7 | 2.1 | 1.0 | 1.7 | 0.4 | 1.0 | 0.1 | 1.2 |
| (DGLA) | | | | | | | | |
| C20:4ω6 | 3.3 | 18.2 | 8.5 | 2.9 | 1.5 | 3.7 | 2.9 | 8.6 |
| (ARA) | | | | | | | | |
| C20:3ω3 | | | | | | | | 0.1 |
| (ETrA) | | | | | | | | |
| C22:0 | 0.0 | 0.2 | 4.5 | 1.1 | 0.9 | 0.0 | 0.0 | 0.4 |
| C22:1 | | | | | | | | 0.1 |
| C20:5ω3 | 0.2 | 0.9 | 0.0 | 0.0 | 0.1 | 0.8 | 1.5 | 0.4 |
| (EPA) | | | | | | | | |
| C22:4ω6 | 0.4 | 2.5 | 0.8 | 2.4 | 0.0 | 0.1 | 0.7 | 1.2 |
| (DTA) | | | | | | | | |
| C24:0 | 0.1 | 0.4 | 4.0 | 1.8 | 1.2 | 0.4 | 0.0 | 0.5 |
| C24:1 | 0.0 | 0.0 | 5.6 | 1.4 | 0.1 | 0.2 | 0.0 | 0.5 |
| C22:5ω3 | 0.4 | 2.3 | 0.2 | 1.3 | 0.3 | 0.2 | 0.6 | 0.9 |
| (DPA) | | | | | | | | |
| C22:6ω3 | 0.0 | 0.0 | 0.0 | 0.0 | 0.1 | 0.0 | 0.0 | 0.4 |
| (DHA) | | | | | | | | |
| Total saturates | 36.1 | 14.9 | 72. | 58.8 | 72.7 | 55 | 73.9 | 27.2 |
| Total MUFA | 32.2 | 37.6 | 13.2 | 22.6 | 18.9 | 24.1 | 13.7 | 35.2 |
| Total ω6 | 30.4 | 44.2 | 14.1 | 17.4 | 7.4 | 18.7 | 10.2 | 33.1 |
| PUFA | | | | | | | | |
| Total ω3 | 1.3 | 3.3 | 0.5 | 1.4 | 0.9 | 1.9 | 2 | 2.1 |
| PUFA | | | | | | | | |
| % of total PL | 57.9 | 29.7 | 4.5 | 1.7 | 0.6 | 0.5 | 0.5 | 0.4 |

TABLE 6
Fatty acid composition and proportions of individual phospholipid
classes in polar lipid of beef. Figures for each fatty acid are
the percentage of the total fatty acid content of the lipid class.

| Fatty acid | PC | PE | PI | PS | LPC | PA | PG |
|---|---|---|---|---|---|---|---|
| C10:0 | 0.3 | 0.0 | 0.1 | 0.0 | 2.0 | 0.0 | 0.0 |
| C12:0 | 0.1 | 0.2 | 0.3 | 0.7 | 1.9 | 0.4 | 4.1 |
| C14:0 | 0.2 | 0.1 | 0.3 | 1.7 | 1.3 | 0.3 | 2.0 |
| C15:0 | 0.2 | 0.5 | 0.3 | 1.4 | 1.8 | 0.3 | 2.8 |
| C16:0 | 29.1 | 4.8 | 9.7 | 16.3 | 27.3 | 2.5 | 28.1 |
| C16:1Δ7 | 0.7 | 1.8 | 0.3 | 3.3 | 2.6 | 0.4 | 2.9 |
| C16:1Δ9 | 0.7 | 0.4 | 0.1 | 0.7 | 0.0 | 1.7 | 1.4 |
| C17:1Δ9 | 0.1 | 2.4 | 0.0 | 3.0 | 0.0 | 0.7 | 4.5 |
| C18:0 (stearate) | 3.7 | 21.8 | 57.5 | 39.2 | 20.0 | 2.7 | 24.0 |
| C18:1Δ9 (oleate) | 24.0 | 7.6 | 3.5 | 8.3 | 13.3 | 4.8 | 5.9 |
| C18:1Δ11 | 1.9 | 1.2 | 2.9 | 0.6 | 0.0 | 6.8 | 2.5 |
| C18:2 (LA) | 25.7 | 20.8 | 3.6 | 8.9 | 21.4 | 74.2 | 13.2 |
| C18:3ω6 (GLA) | 0.4 | 0.3 | 0.1 | 0.7 | 2.8 | 0.3 | 1.4 |
| C18:3ω3 (ALA) | 0.8 | 0.5 | 0.0 | 0.0 | 0.0 | 1.1 | 0.0 |
| C20:0 | 0.1 | 0.1 | 0.6 | 2.5 | 0.0 | 0.0 | 1.7 |
| C20:1Δ11 | 0.1 | 0.1 | 0.0 | 0.0 | 0.0 | 0.3 | 0.0 |
| C20:2ω6 (EDA) | 0.2 | 0.1 | 0.3 | 0.9 | 0.0 | 0.4 | 0.0 |
| C20:3ω6 (DGLA) | 2.9 | 4.9 | 2.2 | 2.1 | 2.2 | 0.9 | 1.5 |
| C20:4ω6 (ARA) | 6.0 | 23.0 | 6.5 | 2.1 | 3.6 | 0.8 | 4.1 |
| C20:3ω3 (ETrA) | 0.1 | 0.2 | 0.0 | 0.0 | 0.0 | 0.0 | 0.0 |
| C22:0 | 0.0 | 0.1 | 4.1 | 2.1 | 0.0 | 0.2 | 0.0 |
| C20:5ω3 (EPA) | 0.5 | 1.6 | 0.4 | 0.0 | 0.0 | 0.0 | 0.0 |
| C22:4ω6 (DTA) | 0.8 | 2.9 | 1.0 | 1.5 | 0.0 | 0.0 | 0.0 |
| C24:0 | 0.1 | 0.3 | 4.0 | 1.1 | 0.0 | 0.2 | 0.0 |
| C24:1 | 0.0 | 0.1 | 1.2 | 0.0 | 0.0 | 0.0 | 0.0 |
| C22:5ω3 (DPA) | 1.2 | 4.0 | 0.6 | 0.9 | 0.0 | 0.3 | 0.0 |
| C22:6ω3 (DHA) | 0.1 | 0.3 | 0.4 | 1.7 | 0.0 | 0.7 | 0.0 |
| % of total PL | 49.7 | 34.3 | 7.0 | 2.1 | 0.5 | 5.4 | 0.8 |
| Total saturates | 33.8 | 27.9 | 76.9 | 65.1 | 54.3 | 6.6 | 62.6 |
| Total monounsaturates | 27.5 | 13.6 | 8.1 | 16.0 | 15.9 | 14.8 | 17.2 |
| Total ω6 fatty acids | 36.0 | 52.0 | 13.6 | 16.3 | 29.8 | 76.5 | 20.2 |
| Total ω3 fatty acids | 2.8 | 6.6 | 1.4 | 2.6 | 0.0 | 2.1 | 0.0 |
| % of total PL | 49.8 | 34.4 | 7 | 2.1 | 0.5 | 5.4 | 0.9 |

Experiment 1—Extraction of Lipids from S. cerevisiae

In a first experiment aiming to test lipid extraction efficiency from yeast cells using ultrasonication for cell disruption in the presence of either a KCl solution or methanol, S. cerevisiae strain INVSc1 was grown in 5 ml of YPD medium for 3 days. The cells were harvested by centrifugation, washed with water and freeze dried as described in Example 1. Identical dried cell pellets of about 25 mg in 2 ml tubes were treated in four ways:
1A. Homogenization in KCl solution, lipid extraction using chloroform/methanol.
1B. Homogenization in KCl solution, sonication for 5 min, lipid extraction using chloroform/methanol.
2A. Homogenization in methanol, lipid extraction using chloroform/methanol/KCl.
2B. Homogenization in methanol, 5 min sonication, lipid extraction using chloroform/methanol/KCl.

In method 1A, 0.3 ml 1M KCl was added to the tube and the cells were disrupted using zirconium beads (Catalog No. ZROB05, Next Advance, Inc., USA) and a Bullet Blender Blue (Next Advance, Inc. USA) at speed 8 for 3 min, followed by addition of 0.4 ml methanol and 0.8 ml chloroform. The mixture was shaken for 5 min and centrifuged for 5 min at 10,000 g. The lower phase containing lipid was transferred to a glass vial. For method 1B, the only difference was an additional step of ultrasonication of the mixture using a water bath sonicator (Bransonic M2800H-E, Branson Ultrasonic Corporation, USA) for 5 min after addition of the methanol and before addition of chloroform. In method 2A, 0.3 ml methanol was added to the tube containing yeast cells and zirconium beads and homogenized in the Bullet Blender, followed by addition of 0.3 ml 1 M KCl, 0.1 ml methanol and 0.8 ml chloroform. The mixture was shaken, centrifuged and lower phase was collected as before. Method 2B was the same as 2A except that an ultrasonication was carried out after cell disruption in the Bullet Blender.

For each sample, the solvent was evaporated from the lipid sample under a flow of nitrogen gas and the extracted lipid dissolved in a measured volume of chloroform. To measure the amount of extracted lipid in each sample, a measured aliquot of the lipid in chloroform was transferred to a GC vial having a PTTE-lined screw cap. After evaporation of the chloroform under nitrogen gas, a known amount of triheptadecanoin (Nu-Chek Prep, Inc., Catalog No. T-155, Waterville, MN, USA) was added to the vial. The fatty acids in each lipid sample were converted to FAME and measured by GC as described in Example 1. The peak areas were integrated and compared to the known amount of heptadecanoin to calculate the amount of fatty acids in the extracted lipids.

As a control to measure the total amount of lipid present in the cells prior to extraction, all of the lipids in duplicate, identical cell pellets were converted to FAME by direct methylation with methanolic-HCl together with triheptadecanoin and analysed by GC. The average of the two controls provided the total fatty acid content, taken as 100% of the cellular fatty acid content. Comparison of the amount of total fatty acid content in the extracted lipids and the cellular fatty acid content provided the extraction efficiency for the four tested methods.

Table 7 provides the data from this experiment. Among the four methods tested, method 2B provided the most efficient lipid extraction from the freeze-dried S. cerevisiae cells, yielding 62.4% of the total cellular fatty acid content. Method 2B included cell disruption in methanol with the zirconium beads and bullet blender and then ultrasonication. On the other hand, method 1B yielded 26.2% lipid extraction efficiency, with homogenisation in KCl solution with ultrasonication for cell disruption. Methods 1A and 2A did not use ultrasonication and yielded lower lipid extraction efficiency.

### Experiment 2.

Another experiment was carried out to estimate lipid extraction efficiency with a larger cell sample and to compare with a method where cells were disrupted in a mixture of chloroform/methanol (2/1, v/v). Dry cell pellets of about 47 mg and 0.5 g zirconium beads were transferred to 2 ml Eppendorf tubes. In method 3A, the efficiency of lipid extraction using ultrasonication was tested. For this, 0.4 ml methanol was added to the tube and the mixture was treated with ultrasonication in a water bath at 40° C. for 10 min. Then, 0.3 ml 1 M KCl and 0.8 ml chloroform were added to the tube and the mixture vortexed for 5 min, followed by centrifugation for 5 min at 10,000 g. The lower phase was collected in a glass vial. Lipid was extracted a second time from the upper phase by adding 0.8 ml chloroform and vortexing the mixture for 5 min, followed by centrifugation and collection of the lower phase which was combined with the first extract in the glass vial. Method 3B used both the zirconium beads and Bullet Blender at speed 8 for 5 min and ultrasonication for 10 min for cell disruption in 0.4 ml methanol, otherwise was the same as method 3B. Method 4 tested cell disruption in a mixture of chloroform/methanol (2/1, v/v) rather than methanol. Extracted lipids were treated and quantitated as for Experiment 1. As in Experiment 1, direct methylation of fatty acids in the cell samples provided the total fatty acid content, taken as 100%.

The data are presented in Table 7. When cells were disrupted in methanol with sonication (Method 3A), 27% of the total lipid content lipid was extracted from the cells. Cellular disruption in methanol using the bullet blender and additionally by sonication yielded 46.4% of the total lipid. On the other hand, cellular disruption in the mixture of chloroform/methanol (2/1, v/v), followed by sonication yielded the lowest level of extracted lipid from S. cerevisiae.

### Experiment 3

A third experiment compared lipid extraction efficiencies from S. cerevisiae cells using glass beads, zirconium beads or metal balls, and using the bead beater or vortexing for the homogenisation of the cells in methanol. Cells from 10 ml cultures were obtained as for the previous experiments and identical cell pellets were treated. Glass beads, zirconium beads or metal balls were added to the tubes and either vortexed or mixed using the bullet blender, as follows.
Method 5: 0.3 ml methanol, 0.5 g glass beads (Catalog No. G8772, Sigma) and two 1 mm metal balls were added to a tube containing the cell pellet, vortexed for 10 min using the Vibramax.
Method 6: 0.3 ml methanol, 0.5 g zirconium beads (Catalog No. ZROB05, Next Advance, Inc., USA) and two 1 mm metal balls were added to the second tube containing the cells and vortexed for 10 min.
Method 7: 0.3 ml methanol and 0.5 g zirconium beads were added to the third tube containing the cell pellet and vortexed for 10 min.
Method 8: 0.3 ml methanol and 0.5 g zirconium beads were added to the fourth tube containing the cell pellet and shaken in a TissueLyser II (Qiagen Inc., Germantown, MD, USA) for 3 min at 25 rpm/sec.

After the homogenisation, 0.4 ml of 1 M KCl, 0.1 ml methanol and 0.8 ml chloroform were added to each tube and the mixtures vortexed for another 5 min. The mixtures were centrifuged at 10,000 g for 5 min and the lower, chloroform phase was transferred to a glass vial. The extracted lipid samples were dried and the fatty acids converted to FAME and quantitated by GC as in the previous experiments.

During the lipid extraction processes, cell debris accumulated at the interphase after the centrifugation of the mixtures. To measure the lipid remaining in the cell debris and so determine the total lipid content, the cell debris was dried in a freeze dryer. A known amount of triheptadecanoin was added and the fatty acids converted to FAME using 0.7 ml methanolic-HCl with incubation at 80° C. for 2 h. FAME were quantitated by GC as before.

The data are presented in Table 7. The most efficient extraction was with method 8, using zirconium beads with the bead beater, extracting 66.5% of the total fatty acid content. Methods 5-7 yielded less extracted lipid than method 8 (Table 7). The efficiency of lipid extraction using the bead beater (method 8) was similar to methods 2B and 3B which involved cell disruption using the bullet blender and sonication. The fatty acid composition of the lipid remaining in the cell debris after the first extraction was the same as for the extracted lipid.

Experiment 4. Extraction of Lipids from Y. lipolytica

For many analyses in Y. lipolytica where the primary purpose was to determine the fatty acid composition of the cells and maximal extraction efficiency was not needed, the inventors decided to routinely use a simpler method that was more suited to high throughput of samples, yet provided sufficient extracted lipid. This conclusion was in view of the observation made in the experiments described above that the fatty acid composition of the extracted lipid was the same as the composition of the residual lipid remaining in the cell debris (Table 7), and so was representative of the total fatty acid content of the cells. In brief, this method, described in Example 1, homogenised dried cell pellets and disrupted the cells in chloroform/methanol (2/1, v/v) solution with zirconium beads using the bullet blender, followed by sonication in the waterbath sonicator and mixing for 20 min. After addition of KCl solution, the mixture was vortexed for 10 min and centrifuged to separate phases. The lower phase was collected. Lipid remaining in the upper phase was extracted using another volume of chloroform and the extracts combined and dried down.

### Example 4. Content and Composition of Polar Lipids from Microbes

The present inventors wanted to determine the content and composition of polar lipids, including phospholipids (PL), from microbes and compare them to animal fats and polar lipids such as those analysed and described in Example 2. The experiments described in this Example also aimed at establishing the fatty acid content and composition, before modification of the microbes or the growth media or both, for production of PLs containing ω6 fatty acids.

TABLE 7
Lipid extraction from S. cerevisiae using different methods of cell disruption.

| | | | | | | | | | | | | | % of |
|---|---|---|---|---|---|---|---|---|---|---|---|---|---|
| | | | | | | | | | | | | Lipid %/ | total |
| | C10:0 | C12:0 | C14:0 | C14:1 | C15:0 | C16:0 | C16:1Δ7 | C16:1Δ9 | C18:0 | C18:1Δ9 | C18:1Δ11 | DCW | lipid |
| Experiment 1 | | | | | | | | | | | | | |
| 1A | 0.9 | 0.8 | 0.7 | 0.3 | 0.2 | 10.5 | 0.6 | 55.1 | 3.7 | 24.5 | 2.8 | 0.7 | 18.8 |
| 1B | 1.0 | 0.9 | 0.8 | 0.3 | 0.2 | 11.0 | 0.6 | 54.1 | 3.9 | 24.5 | 2.7 | 1.0 | 26.2 |
| 2A | 1.1 | 0.9 | 0.9 | 0.3 | 0.2 | 10.2 | 0.7 | 55.0 | 3.6 | 24.4 | 2.6 | 1.9 | 50.3 |
| 2B | 1.1 | 1.0 | 0.9 | 0.4 | 0.2 | 10.4 | 0.7 | 54.4 | 3.7 | 24.6 | 2.6 | 2.3 | 62.4 |
| total | 0.8 | 0.8 | 0.7 | 0.2 | 0.2 | 11.7 | 0.8 | 52.2 | 4.2 | 25.0 | 3.3 | 3.8 | 100% |
| Experiment 2 | | | | | | | | | | | | | |
| 3A | 1.3 | 0.9 | 0.9 | 0.4 | 0.2 | 10.0 | | 54.0 | 3.6 | 26.1 | 2.7 | 1.1 | 27.0 |
| 3B | 1.3 | 0.9 | 0.9 | 0.4 | 0.2 | 10.0 | | 54.0 | 3.5 | 26.4 | 2.6 | 1.9 | 46.4 |
| 4 | 1.6 | 1.0 | 0.9 | 0.4 | 0.2 | 9.3 | | 54.6 | 3.4 | 25.9 | 2.7 | 1.0 | 24.1 |
| total 1 | 1.1 | 0.8 | 0.8 | 0.3 | 0.2 | 11.8 | | 50.7 | 4.3 | 27.5 | 2.5 | 4.1 | 100% |
| total 2 | 1.0 | 0.8 | 0.8 | 0.3 | 0.2 | 11.8 | | 48.7 | 4.6 | 29.3 | 2.4 | 4.2 | 100% |
| Experiment 3 | | | | | | | | | | | | | |
| 5 | 1.6 | 0.9 | 1.1 | 0.4 | 0.4 | 12.7 | 0.8 | 56.3 | 4.1 | 19.9 | 1.8 | 304 | 33.5 |
| 6 | 1.6 | 0.9 | 1.1 | 0.4 | 0.3 | 12.7 | 0.7 | 55.4 | 4.2 | 20.7 | 1.9 | 429 | 50.5 |
| 7 | 1.5 | 0.9 | 1.1 | 0.4 | 0.4 | 12.8 | 0.7 | 55.7 | 4.1 | 20.5 | 1.9 | 376 | 43.9 |
| 8 | 1.4 | 0.9 | 1.1 | 0.4 | 0.3 | 12.9 | 0.7 | 54.3 | 4.4 | 21.5 | 1.9 | 480 | 66.5 |
| Cell | 1.1 | 0.9 | 1.0 | 0.4 | 0.4 | 14.8 | 0.7 | 51.4 | 5.2 | 22.3 | 1.8 | 603 | 66.5 |
| debris 1 | | | | | | | | | | | | | |
| Cell | 1.0 | 0.9 | 1.0 | 0.4 | 0.4 | 14.9 | 0.7 | 50.5 | 5.4 | 23.0 | 1.8 | 420 | 49.5 |
| debris 2 | | | | | | | | | | | | | |
| Cell | 1.0 | 0.9 | 1.0 | 0.4 | 0.4 | 14.6 | 0.7 | 51.0 | 5.2 | 23.1 | 1.8 | 481 | 56.1 |
| debris 3 | | | | | | | | | | | | | |
| Cell | 1.1 | 0.9 | 1.0 | 0.4 | 0.4 | 15.7 | 0.7 | 49.7 | 5.7 | 22.8 | 1.7 | 242 | 33.5 |
| debris 4 | | | | | | | | | | | | | |

### Growth of Microbes and Extraction of Lipids

In order to determine the amount and fatty acid composition of polar lipids including PLs and triacylglycerols (TAG) in microbial cells during their growth cycle, five widely used strains of three different species were selected. These were E. coli strains DH5α and BL21, the oleaginous wild-type Y. lipolytica strain W29, and S. cerevisiae strains INVSc1 and D5A. These species were chosen due to the availability of genetic tools and processes for genetic engineering as well as the depth of knowledge about lipid synthesis and metabolism in these species. Strain D5A was selected as an oleaginous strain of S. cerevisiae (He et al., 2018). These microbes were cultured for up to 7 days, with removal and analysis of samples at different time points. Inoculum cultures were prepared by growing cells overnight in LB medium for E. coli or YPD or SD+Ura media for the yeasts. Samples of these cultures were diluted into 200 ml of the same growth medium in 1 L bottles to provide an initial OD600 of 0.1. The mouth of each bottle was covered by micropore tape and the cultures were shaken for aeration. The E. coli cells were incubated in a shaker at 37° C. at 250 rpm. Yeast cells were grown in the YPD medium containing 2% glucose as carbon source and incubated at 28° C. with shaking at 200 rpm. Samples of 10 ml were removed from each culture at 18 h, 24 h, 2 d, 3 d, 4 d, 5 d, 6 d and 7 d time points. Cells were harvested from the cultures by centrifugation at 3,400 g for 10 min and washed twice with 3 ml each time of de-ionised water and once with 1.5 ml de-ionised water. The cells were transferred to pre-weighed 2 ml tubes and freeze dried for 24 h. The tubes were then re-weighed and the dry cell weights were calculated prior to lipid extraction.

Total cellular lipid was extracted as described in Example 1, using 0.6 ml chloroform/methanol (2/1, v/v) as the extraction solvent in the presence zirconium beads using a bullet blender, followed by sonication in a water bath at 40° C. After mixing the homogenate with 0.3 ml 0.1 M KCl for 10 min, the mixture was centrifuged at 10,000 g for 5 min. The lower phase containing lipid was transferred to a glass vial. Remaining lipid was extracted from the upper phase containing the cell debris with 0.6 ml chloroform for 20 min, centrifugation and the collection of the lower phase as before. The solvent was evaporated from the combined lower phases under a flow of nitrogen gas and the extracted lipid was resuspended in a measured volume of chloroform. Aliquots of lipid extracted from 20 mg dry cell weight were fractionated on a TLC plate using a solvent mixture of hexane/diethylether/acetic acid (70/30/1, v/v/v) to separate TAG and polar lipids, as described in Example 1. The fatty acid composition of the lipid from the TAG and polar lipid spots were determined by GC of FAME produced from the lipids, again as described in Example 1.

Initial Experiment with S. cerevisiae

In an initial experiment, lipid was extracted from cultured cells of S. cerevisiae strain INVSc1 after growth for 1, 2, 3 or 4 days in YPD and SD+Ura media. The data are shown in Table 8, including the extracted lipid yield as a percentage of dry cell weight (DCW). The efficiency of recovery of the TAG and polar lipids in the TLC fractionation was not determined. It was noted that the amount of TAG produced by the INVSc1 cells was low when cultured in YPD medium, while higher in SD+Ura medium. Polar lipid yields were between 0.63% and 1.15% on a dry cell weight basis, but the method was not maximised for efficient extraction. For the fatty acid composition, both fractions contained 47-67% of C16:1Δ9 as the fatty acid present in the greatest amount. Oleic acid (C18:1Δ9) and palmitic acid (C16:0) were the other main fatty acids present, as was a low level of stearic acid (C18:0), while linoleic acid (LA, C18:2Δ9,12) was not present. These data were consistent with published reports (e.g. Itoh and Kaneko, 1974; Stukey et al., 1989; Kamisaka et al., 2015) that reported the presence of 40-55% of C16:1, 30-35% of C18:1Δ9, and lesser amounts of C16:0 and C18:0. These four fatty acids make up almost all of the fatty acid content in many wild-type S. cerevisiae strains. Wild-type strains such as INVSc1 contain only one fatty acid desaturase, a 49-desaturase encoded by the OLE1 gene, which produces the monounsaturated palmitoleic and oleic acids (Stukey et al., 1989).

Like S. cerevisiae, the wild-type fission yeast S. pombe is unable to synthesize LA and other polyunsaturated fatty acids (Ratledge and Evans 1989; Holic et al., 2012). In contrast, other wild-type yeasts such as S. kluyveri and K. lactis have Δ12- and A15-desaturases and can produce LA and ALA.

Fatty Acid Composition in E. coli, Y. lipolytica and S. cerevisiae after Growth for Up to 7 Days

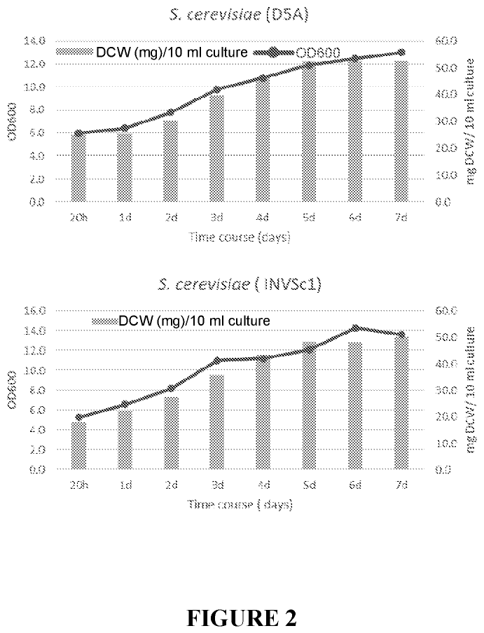

An experiment was carried out with E. coli, S. cerevisiae and Y. lipolytica, sampling the cultures daily up to 4 or 7 days. The growth curves for two S. cerevisiae strains are provided in FIG. 2, showing OD600 and the dry cell weight over the 7 days of culturing. The amount of lipid and fatty acid compositions were determined for both the polar lipid and TAG fractions for each strain at each time point. The data are presented in Table 9 for two E. coli strains, Table 10 for Y. lipolytica strain W29 and Table 11 for S. cerevisiae strains INVSc1 and D5A. The identity of the fatty acid C15:0 (pentadecanoic acid) was confirmed by GC-MS.

The fatty acid composition of the polar lipid of E. coli strain BL21 was similar to that reported by Kanemasa et al. (1967) and Marr and Ingraham (1962) for other, wild-type E. coli strains. As for many other bacteria, E. coli polar lipids contain four types of fatty acids: straight chain saturated fatty acids including C12:0, C14:0, C15:0 and C16:0, straight chain monounsaturated fatty acids including C16:1Δ9 (cis-palmitoleic acid) and C18:1Δ11 (cis-vaccenic acid), branched chain fatty acids, and cyclopropane fatty acids including C17:0c* (cis-9,10-methylene hexadecenoic acid) and C19:0c* (cis-11,12-methylene octadecenoic acid) (Hildebrand and Law, 1964). The presence of C16:0, C16:1, C18:0 and C18:1Δ11 was reported in E. coli strain BL21 by Oldham et al. (2001). The unsaturated fatty acids found in wild-type E. coli are all monoenes of the cis conformation, but do not include oleic acid (Cronan and Vagelos, 1972). The four fatty acid types were all observed in the extracted lipid from BL21, which had about 31-36% C18:1Δ11 and about 7-10% C16:1Δ9, as well as 30-35% of the saturated fatty acid C16:0 (palmitic acid), 10-20% of the cyclopropane fatty acid C17:0c* and 1-5% of C19:0c*. These latter two fatty acids are distinctive for bacterial lipids, being rarely found in animal fats or yeast lipids. They are produced from the corresponding monoenes C16:1Δ9 and C18:1Δ11 through the activity of a cyclopropane fatty acid synthase (CPFAS). Another difference observed with animal fats was that polyunsaturated fatty acids such as LA were not present in wild-type E. coli lipids, and this was observed for BL21 and DH5α. Additionally, oleic acid (C18:1Δ9) was not observed in the E. coli polar lipid but is present at substantial levels in animal and plant lipids.

Strain DH5α exhibited a significantly different fatty acid composition to BL21 in terms of the amounts of some fatty acids in its polar lipid, having considerably less C18:1Δ11 at about 3-8% and less C16:1Δ9, but more C16:0 and considerably more C15:0 and cyclopropane fatty acids. In DH5α, almost half of the total fatty acid content was palmitic acid, which was reported to be located almost exclusively at the sn-1 position of the phospholipid (Cronan and Vagelos, 1972). Hildebrand and Law (1964) reported the presence of cyclopropane fatty acids in E. coli, and they were also observed here in DH5x. As shown in Table 9, pentadecanoic acid, nonadecanoic acid (C19:0) and the cyclopropane fatty acids were observed in the polar lipid fraction. The decrease in DH5α relative to BL21 in the levels of C16:1, and C18:1Δ11 was accompanied by increased amounts of C14:0, C15:0, C16:0 and the cyclopropane fatty acids. Both E. coli strains had less than 2% stearic acid (C18:0) in their lipids. The highest amount of polar lipid was observed at day 2 of culturing, at about 2.7% DCW.

The fatty acid composition of Y. lipolytica (Table 10) was quite different to that of E. coli and S. cerevisiae. A wider range of fatty acids was observed in Y. lipolytica lipid, including, for example, polyunsaturated fatty acids such as LA and longer chain, saturated fatty acids having 20, 22 or 24 carbons, C20:0, C22:0 and C24:0 which were all present in the TAG fraction. C24:0 was generally present and C20:0 and C22:0 absent from the polar lipid fraction. Although Y. lipolytica is an oleaginous microbe, the growth conditions in this experiment using rich YPD medium did not favour high level TAG production, so producing less than about 1% TAG on a dry cell weight basis. TAG continued accumulating at that low level during the 7-day period. The highest level of polar lipid was observed at day 2 of the culture. Palmitic, palmitoleic, oleic and linoleic acids were the major fatty acids in Y. lipolytica. The polar lipid also contained short, medium and long-chain saturated and monounsaturated fatty acids at low levels, together with odd chain fatty acids such as pentadecanoic acid and heptadecenoic acid (Table 10). The identity of the peak for pentadecanoic acid was confirmed by GC-MS. The fatty acid composition was similar to that reported by Carsanba et al. (2020).

The polar lipid and TAG fractions of Y. lipolytica showed significantly different amounts of some fatty acids. In general, the polar lipid contained higher levels of LA and palmitoleic acid (C16:1) than TAG, while the TAG was richer in palmitic, stearic acid and lignoceric acids. In particular, the TAG had much greater levels of the saturated fatty acid stearic acid at about 4-12% compared to less than 1% in the polar lipids, as well as greater amounts of the saturated C20, C22 and C24 fatty acids. The Y. lipolytica polar lipid was easily distinguishable from the E. coli lipid, for example the former had C18:1Δ9 (oleic acid) rather than C18:1Δ11 (vaccenic acid) as the predominant monounsaturated fatty acid. As noted above, E. coli lipid lacked oleic acid.

The polar lipid and TAG fractions from S. cerevisiae strains INVSc1 and D5A contained mostly four fatty acids, the monounsaturated fatty acid palmitoleic acid (C16:1Δ9) and oleic acid (C18:1Δ9) and the saturated fatty acids palmitic acid 9C16:0) and stearic acid (C18:0). These data were consistent with published reports (He et al., 2018). The polar lipid fractions were slightly higher in the saturated fatty acids and lower in the monounsaturated fatty acids relative to the TAG fractions.

### Example 5. Feeding Omega-6 Fatty Acids to Microbes and the Effects on Polar Lipids

The fatty acid composition of meat lipids revealed higher proportions of ω6 fatty acids such as GLA, DGLA, ARA and DTA in the polar lipid fraction, including in the phospholipid (PL), compared to the TAG fraction (Example 2). The inventors hypothesized that these fatty acids might be involved in the generation of aromas from meats such as beef and pork. The inventors therefore attempted to produce animal-like PL by incorporation of ω6 fatty acids into microbial PL. This was initially done by feeding ω6 fatty acids to the microbes during growth and then extracting the lipids and fractionating them to isolate the polar lipids, including the PL.

TABLE 8
Fatty acid composition of TAG and polar lipid fractions of S. cerevisiae
strain INVSc1 cells during culturing for 1 to 4 days.

| | | | | | | | | |
|---|---|---|---|---|---|---|---|---|
| Lipid | | | | | | | | |
| fraction/ | | | | | | | | |
| days of | | | | | | | | |
| culture | C10:0 | C12:0 | C14:0 | C14:1Δ9 | C15:0 | C16:0 | C16:1T | C16:1Δ7 |

YPD medium

| | | | | | | | | |
|---|---|---|---|---|---|---|---|---|
| TAG-1 | 1.0 | 1.3 | 1.4 | 0.4 | 0.0 | 10.4 | 0.3 | 0.0 |
| day | | | | | | | | |
| TAG-2 | 1.0 | 1.2 | 0.7 | 0.0 | 0.0 | 7.2 | 0.0 | 0.0 |
| days | | | | | | | | |
| TAG-3 | 0.6 | 0.7 | 0.9 | 0.0 | 0.0 | 9.4 | 0.0 | 0.0 |
| days | | | | | | | | |
| TAG-4 | 0.2 | 0.5 | 0.9 | 0.0 | 0.0 | 11.9 | 0.0 | 0.0 |
| days | | | | | | | | |
| Polar-1 | 0.9 | 0.6 | 1.2 | 0.3 | 0.3 | 20.0 | 0.7 | 0.0 |
| day | | | | | | | | |
| Polar-2 | 0.7 | 0.3 | 0.8 | 0.2 | 0.4 | 13.7 | 0.7 | 0.0 |
| days | | | | | | | | |
| Polar-3 | 0.3 | 0.2 | 0.7 | 0.2 | 0.3 | 11.8 | 0.7 | 0.0 |
| days | | | | | | | | |
| Polar-4 | 0.1 | 0.1 | 0.6 | 0.1 | 0.3 | 11.5 | 0.7 | 0.0 |
| days | | | | | | | | |

SD + Ura medium

| | | | | | | | | |
|---|---|---|---|---|---|---|---|---|
| TAG-1 | 0.0 | 0.5 | 0.7 | 0.2 | 0.0 | 8.9 | 0.4 | 0.0 |
| day | | | | | | | | |
| TAG-2 | 0.0 | 0.2 | 0.5 | 0.1 | 0.3 | 13.9 | 0.4 | 0.0 |
| days | | | | | | | | |
| TAG-3 | 0.0 | 0.2 | 0.5 | 0.1 | 0.3 | 17.2 | 0.4 | 0.0 |
| days | | | | | | | | |
| TAG-4 | 0.0 | 0.2 | 0.5 | 0.0 | 0.3 | 19.3 | 0.4 | 0.0 |
| days | | | | | | | | |
| Polar-1 | 0.3 | 0.3 | 0.7 | 0.2 | 0.2 | 13.4 | 0.7 | 0.0 |
| day | | | | | | | | |
| Polar-2 | 0.2 | 0.1 | 0.4 | 0.1 | 0.3 | 12.9 | 0.7 | 0.0 |
| days | | | | | | | | |
| Polar-3 | 0.1 | 0.1 | 0.3 | 0.1 | 0.3 | 12.7 | 0.7 | 0.0 |
| days | | | | | | | | |
| Polar-4 | 0.3 | 0.1 | 0.3 | 0.0 | 0.3 | 14.4 | 0.8 | 0.0 |
| days | | | | | | | | |

| Lipid | | | | | | |
|---|---|---|---|---|---|---|
| fraction/ | | | | | | TAG or |
| days of | | | | | | PL % per |
| culture | C16:1Δ9 | C18:0 | C18:1Δ9 | C18:1Δ11 | C18:2Δ9, 12 | DCW |
| YPD medium | | | | | | |
| TAG-1 | 57.2 | 3.6 | 21.1 | 3.2 | 0.0 | 0.26 |
| day | | | | | | |
| TAG-2 | 67.7 | 3.1 | 19.1 | 0.0 | 0.0 | 0.28 |
| days | | | | | | |
| TAG-3 | 60.4 | 4.0 | 24.0 | 0.0 | 0.0 | 0.52 |
| days | | | | | | |
| TAG-4 | 57.4 | 3.8 | 25.3 | 0.0 | 0.0 | 0.66 |
| days | | | | | | |
| Polar-1 | 51.7 | 2.4 | 20.2 | 1.8 | 0.0 | 0.75 |
| day | | | | | | |
| Polar-2 | 54.1 | 3.1 | 23.1 | 2.7 | 0.0 | 0.63 |
| days | | | | | | |
| Polar-3 | 53.2 | 3.0 | 27.1 | 2.6 | 0.0 | 1.08 |
| days | | | | | | |
| Polar-4 | 51.8 | 2.8 | 29.4 | 2.6 | 0.0 | 1.15 |
| days | | | | | | |
| SD + Ura medium | | | | | | |
| TAG-1 | 56.5 | 4.8 | 25.0 | 3.0 | 0.0 | 0.31 |
| day | | | | | | |
| TAG-2 | 53.8 | 3.6 | 24.4 | 2.8 | 0.0 | 0.85 |
| days | | | | | | |
| TAG-3 | 51.5 | 4.5 | 22.7 | 2.7 | 0.0 | 1.21 |
| days | | | | | | |
| TAG-4 | 48.1 | 5.8 | 22.8 | 2.8 | 0.0 | 1.69 |
| days | | | | | | |
| Polar-1 | 51.5 | 4.1 | 26.3 | 2.2 | 0.0 | 0.94 |
| day | | | | | | |
| Polar-2 | 50.0 | 3.7 | 28.9 | 2.8 | 0.0 | 0.65 |
| days | | | | | | |
| Polar-3 | 48.5 | 3.4 | 30.7 | 3.1 | 0.0 | 0.89 |
| days | | | | | | |
| Polar-4 | 47.3 | 4.1 | 29.5 | 3.0 | 0.0 | 0.94 |
| days | | | | | | |

TABLE 9
Fatty acid composition and amount of polar lipid in E. coli during culturing in LB medium.

| | | | | | | | | | | |
|---|---|---|---|---|---|---|---|---|---|---|
| | | | | | | | | | | Polar |
| | | | | | | | | | | lipid % |
| C12:0 | C14:0 | C15:0 | C16:0 | C16:1Δ9 | C18:0 | C18:1Δ11 | C19:0 | C17:0c* | C19:0c* | (% of DCW) |

E. coli strain DH5α

| | | | | | | | | | | | |
|---|---|---|---|---|---|---|---|---|---|---|---|
| 20 h | 0.6 | 5.6 | 3.8 | 47.9 | 6.4 | 0.8 | 7.8 | 4.6 | 18.3 | 4.1 | 1.4 |
| 1 d | 0.9 | 6.1 | 4.3 | 46.9 | 7.3 | 0.7 | 7.5 | 4.4 | 17.7 | 4.1 | 2.0 |
| 2 d | 0.6 | 5.8 | 8.1 | 46.4 | 2.6 | 0.3 | 4.6 | 6.3 | 20.3 | 4.9 | 2.7 |
| 3 d | 0.5 | 5.4 | 9.0 | 45.8 | 2.1 | 1.2 | 4.2 | 6.4 | 20.6 | 4.9 | 1.9 |
| 4 d | 0.4 | 5.0 | 10.6 | 44.3 | 1.6 | 1.2 | 3.7 | 6.9 | 21.3 | 5.0 | 2.4 |
| 5 d | 0.3 | 4.6 | 12.0 | 44.7 | 1.6 | 1.6 | 3.6 | 9.2 | 18.2 | 4.2 | 1.1 |
| 6 d | 0.3 | 4.1 | 13.2 | 41.9 | 2.0 | 1.5 | 3.2 | 8.9 | 20.2 | 4.6 | 1.0 |
| 7 d | 0.5 | 4.5 | 14.8 | 39.4 | 4.1 | 0.2 | 3.3 | 8.7 | 19.9 | 4.0 | 1.1 |

E. coli strain BL21

| | | | | | | | | | | | |
|---|---|---|---|---|---|---|---|---|---|---|---|
| 20 h | 0.5 | 3.7 | 0.2 | 35.4 | 8.9 | 1.0 | 31.0 | 3.0 | 11.7 | 4.5 | 1.9 |
| 1 d | 2.0 | 4.5 | 0.2 | 33.3 | 9.3 | 0.9 | 31.5 | 2.8 | 11.4 | 4.0 | 1.8 |
| 2 d | 0.8 | 2.4 | 0.4 | 30.3 | 8.3 | 1.0 | 36.2 | 4.4 | 13.8 | 2.3 | 2.0 |
| 3 d | 0.5 | 1.7 | 0.5 | 30.9 | 7.4 | 1.2 | 35.0 | 4.9 | 15.7 | 2.1 | 1.3 |
| 4 d | 0.5 | 1.6 | 0.5 | 30.7 | 7.0 | 1.2 | 35.2 | 5.4 | 15.9 | 2.0 | 1.5 |
| 5 d | 1.0 | 2.1 | 0.4 | 30.6 | 6.9 | 1.4 | 36.5 | 6.5 | 12.8 | 1.7 | 0.5 |
| 6 d | 1.4 | 2.4 | 0.4 | 30.1 | 7.2 | 1.3 | 36.2 | 5.9 | 13.3 | 1.8 | 0.4 |
| 7 d | 1.3 | 2.3 | 0.4 | 29.7 | 9.7 | 1.0 | 35.7 | 6.0 | 12.1 | 1.6 | 0.9 |

*Cyclopropane fatty acids; DCW, dry cell weight.

TABLE 10
Fatty acid composition of TAG and polar lipid fractions from Y. lipolytica during culturing in YPD medium.

| | C12:0 | C12:1 | C14:0 | C15:0 | C16:0 | C16:1Δ7 | C16:1Δ9 | C17:1 | C18:0 | C18:1Δ9 | C18:1Δ11 |
|---|---|---|---|---|---|---|---|---|---|---|---|
| Polar lipid | | | | | | | | | | | |
| 20 h | 0.1 | 0.1 | 0.3 | 0.7 | 11.6 | 0.9 | 12.1 | 1.8 | 0.7 | 55.8 | 0.9 |
| 1 d | 0.1 | 0 | 0.2 | 0.5 | 9.8 | 0.9 | 12.5 | 1.8 | 0.5 | 57.0 | 1.1 |
| 2 d | 0.2 | 0 | 0.4 | 0.5 | 10.1 | 1.4 | 13.9 | 2.7 | 0.4 | 55.0 | 2.0 |
| 3 d | 0.1 | 0.1 | 0.4 | 0.3 | 10.8 | 2.2 | 12.7 | 3.0 | 0.5 | 53.5 | 3.2 |
| 4 d | 0.2 | 0.3 | 0.5 | 0.2 | 11.5 | 2.4 | 12.1 | 2.7 | 0.5 | 53.2 | 3.7 |
| 5 d | 0.2 | 0.2 | 0.8 | 0.2 | 13.6 | 3.2 | 13.6 | 2.1 | 0.6 | 49.6 | 3.7 |
| 6 d | 0.2 | 0 | 0.5 | 0.2 | 11.2 | 3.2 | 13.7 | 1.9 | 0.4 | 48.4 | 3.0 |
| 7 d | 0.1 | 0 | 0.5 | 0.2 | 9.7 | 2.6 | 13.6 | 1.6 | 0.3 | 47.5 | 2.7 |
| Triacylglycerols | | | | | | | | | | | |
| 20 h | 0.2 | 0.4 | 0.4 | 0.5 | 10.3 | 0.7 | 10.7 | 1.6 | 5.6 | 51.1 | 0.8 |
| 1 d | 0 | 0.2 | 0 | 0.6 | 15.2 | 0.6 | 8.6 | 1.2 | 12.4 | 41. | 0.9 |
| 2 d | 0 | 0 | 0.2 | 0.5 | 15.4 | 0.8 | 9.5 | 1.6 | 11.0 | 43.3 | 1.1 |
| 3 d | 0 | 1.1 | 0.1 | 0.4 | 15.8 | 1.2 | 8.1 | 1.7 | 11.8 | 41.5 | 1.4 |
| 4 d | 0 | 0.4 | 0.1 | 0.2 | 13.8 | 1.4 | 8.7 | 1.0 | 10.2 | 44.9 | 1.7 |
| 5 d | 0.1 | 1 | 0.1 | 0.2 | 10.1 | 1.8 | 11.2 | 1.8 | 6.5 | 52.1 | 1.9 |
| 6 d | 0.1 | 0.8 | 0.1 | 0.2 | 7.8 | 1.2 | 11.8 | 1.5 | 5.2 | 55.4 | 2.7 |
| 7 d | 0 | 0 | 0.1 | 0.1 | 8.6 | 0.9 | 12.2 | 1.0 | 4.3 | 59.5 | 2.8 |

| | | | | | | | TAG or |
|---|---|---|---|---|---|---|---|
| | | | | | | | Polar % |
| | C18:2Δ9, 12 | C20:0 | C20:1Δ11 | C20:2Δ11, 14 | C22:0 | C24:0 | per DCW |
| Polar lipid | | | | | | | |
| 20 h | 14.6 | 0 | 0.2 | 0 | 0 | 0.3 | 0.4 |
| 1 d | 15.1 | 0 | 0.2 | 0 | 0 | 0.2 | 1.8 |
| 2 d | 13.2 | 0 | 0.1 | 0 | 0 | 0.2 | 3.3 |
| 3 d | 12.8 | 0 | 0.1 | 0 | 0 | 0.2 | 2.0 |
| 4 d | 12.4 | 0 | 0.2 | 0 | 0 | 0.3 | 1.8 |
| 5 d | 11.3 | 0 | 0.4 | 0.1 | 0 | 0.4 | 1.6 |
| 6 d | 15.7 | 0 | 0.9 | 0.2 | 0 | 0.5 | 1.9 |
| 7 d | 20.2 | 0 | 0.8 | 0.2 | 0 | 0.2 | 1.2 |
| Triacylglycerols | | | | | | | |
| 20 h | 13.6 | 0.2 | 0 | 0 | 0.4 | 3.5 | 0.1 |
| 1 d | 8.8 | 0.9 | 0 | 0 | 1.0 | 8.2 | 0.2 |
| 2 d | 7.9 | 0.5 | 0.1 | 0 | 0.7 | 7.3 | 0.7 |
| 3 d | 7.1 | 0.6 | 0 | 0 | 0.8 | 8.6 | 0.6 |
| 4 d | 7.2 | 0.5 | 0.2 | 0 | 0.6 | 8.1 | 0.8 |
| 5 d | 6.8 | 0.4 | 0.4 | 0 | 0.4 | 5.4 | 1.1 |
| 6 d | 7.9 | 0.3 | 0.6 | 0.1 | 0.4 | 4.2 | 1.1 |
| 7 d | 7.7 | 0.2 | 0.4 | 0.1 | 0.2 | 1.7 | 0.9 |

TABLE 11
Fatty acid composition of polar lipid and TAG fractions from S. cerevisiae
strains INVSc1 and D5A during culturing in YPD medium for up to 7 days.

| | | | | | | | | | |
|---|---|---|---|---|---|---|---|---|---|
| | | | | | | | | | Amount |
| | | | | | | | | | recovered |
| C12:0 | C14:0 | C15:0 | C16:0 | C16:1Δ9 | C18:0 | C18:1Δ9 | C18:1Δ11 | C18:2Δ9, 12 | (% of DCW) |

Polar lipid - strain INVSc1

| | | | | | | | | | | |
|---|---|---|---|---|---|---|---|---|---|---|
| 20 h | 1.0 | 1.4 | 0.5 | 17.6 | 54.8 | 2.2 | 21.2 | 1.3 | 0.0 | 1.0 |
| 1 d | 0.7 | 0.8 | 0.6 | 13.2 | 54.8 | 3.0 | 25.1 | 1.9 | 0.0 | 0.8 |
| 2 d | 0.3 | 0.5 | 0.6 | 11.1 | 52.9 | 4.3 | 28.2 | 2.2 | 0.0 | 1.0 |
| 3 d | 0.2 | 0.4 | 0.4 | 11.7 | 51.0 | 4.8 | 29.9 | 1.6 | 0.0 | 0.8 |
| 4 d | 0.1 | 0.4 | 0.4 | 11.5 | 50.4 | 5.1 | 30.4 | 1.6 | 0.0 | 0.9 |
| 5 d | 0.1 | 0.4 | 0.5 | 12.0 | 49.7 | 5.4 | 30.3 | 1.6 | 0.0 | 0.7 |
| 6 d | 0.1 | 0.3 | 0.5 | 11.1 | 49.6 | 5.5 | 31.3 | 1.7 | 0.0 | 0.8 |
| 7 d | 0.1 | 0.3 | 0.5 | 10.9 | 49.0 | 5.8 | 31.8 | 1.6 | 0.0 | 0.8 |

Triacylglycerols - strain INVSc1

| | | | | | | | | | | |
|---|---|---|---|---|---|---|---|---|---|---|
| 20 h | 1.7 | 2.7 | 0.6 | 14.0 | 58.2 | 3.7 | 17.7 | 1.4 | 0.0 | 0.3 |
| 1 d | 0.9 | 1.3 | 0.6 | 9.5 | 56.2 | 5.5 | 23.7 | 2.4 | 0.0 | 0.3 |
| 2 d | 0.3 | 0.9 | 0.6 | 10.8 | 49.2 | 5.9 | 30.4 | 1.9 | 0.0 | 0.5 |
| 3 d | 0.2 | 1.2 | 0.5 | 13.9 | 45.2 | 6.1 | 31.5 | 1.4 | 0.0 | 1.2 |
| 4 d | 0.2 | 1.0 | 0.5 | 14.1 | 45.3 | 6.5 | 30.9 | 1.4 | 0.0 | 1.5 |
| 5 d | 0.4 | 2.0 | 1.1 | 16.2 | 48.8 | 5.5 | 24.5 | 1.4 | 0.0 | 1.5 |
| 6 d | 0.1 | 0.7 | 0.5 | 14.0 | 45.3 | 7.2 | 30.6 | 1.5 | 0.0 | 1.6 |
| 7 d | 0.1 | 0.7 | 0.5 | 13.5 | 45.3 | 7.5 | 30.8 | 1.5 | 0.0 | 3.0 |

Polar lipid - strain D5A

| | | | | | | | | | | |
|---|---|---|---|---|---|---|---|---|---|---|
| 20 h | 0.4 | 1.9 | 0.2 | 19.5 | 49.3 | 2.1 | 25.6 | 0.8 | 0.0 | 0.8 |
| 1 d | 0.5 | 1.8 | 0.2 | 19.1 | 48.2 | 2.4 | 26.5 | 1.1 | 0.0 | 0.9 |
| 2 d | 0.3 | 1.2 | 0.3 | 15.6 | 46.5 | 3.6 | 31.0 | 1.5 | 0.0 | 1.2 |
| 3 d | 0.2 | 0.7 | 0.2 | 11.8 | 44.4 | 4.9 | 36.2 | 1.6 | 0.0 | 1.2 |
| 4 d | 0.1 | 0.5 | 0.2 | 10.5 | 41.6 | 5.8 | 39.6 | 1.7 | 0.0 | 1.5 |
| 5 d | 0.1 | 0.4 | 0.1 | 10.7 | 38.4 | 6.6 | 42.0 | 1.6 | 0.0 | 1.4 |
| 6 d | 0.1 | 0.4 | 0.2 | 9.8 | 37.8 | 7.1 | 43.2 | 1.5 | 0.0 | 1.3 |
| 7 d | 0.1 | 0.4 | 0.2 | 9.4 | 37.4 | 7.3 | 43.8 | 1.5 | 0.0 | 1.3 |

Triacylglycerols - strain D5A

| | | | | | | | | | | |
|---|---|---|---|---|---|---|---|---|---|---|
| 20 h | 1.4 | 3.6 | 0.2 | 11.4 | 50.7 | 2.2 | 29.7 | 0.8 | 0.0 | 2.5 |
| 1 d | 1.4 | 3.3 | 0.2 | 10.1 | 52.5 | 2.3 | 29.4 | 0.9 | 0.0 | 2.3 |
| 2 d | 0.7 | 2.0 | 0.2 | 10.7 | 50.4 | 4.0 | 30.5 | 1.4 | 0.0 | 2.6 |
| 3 d | 0.3 | 1.0 | 0.2 | 10.0 | 48.9 | 4.7 | 33.2 | 1.7 | 0.0 | 2.2 |
| 4 d | 0.2 | 0.7 | 0.2 | 8.4 | 48.5 | 4.7 | 35.7 | 1.6 | 0.0 | 2.3 |
| 5 d | 0.1 | 0.6 | 0.1 | 8.6 | 43.8 | 5.8 | 39.3 | 1.6 | 0.0 | 3.5 |
| 6 d | 0.1 | 0.5 | 0.1 | 7.9 | 44.1 | 5.5 | 40.2 | 1.5 | 0.0 | 3.2 |
| 7 d | 0.1 | 0.5 | 0.1 | 7.6 | 43.8 | 5.5 | 40.8 | 1.5 | 0.0 | 2.8 |

As a source of ARA in feeding experiments, an ARA-containing oil was obtained from Jinan Boss Chemical Industry Co., Ltd (China), having 50% ARA in its total fatty acid content (Table 12). In a preliminary experiment, some of the oil was dissolved in ethanol and added to the culture medium of Y. lipolytica strain W29 to a final concentration of 1, 2 or 4 mg oil/ml culture. The base medium used was YPD with 1% tergitol (NP40) added in an attempt to solubilize the oil and the starting OD600 was 0.1 After 48 h incubation at 28° C. with shaking, cells were harvested and analysed for ARA incorporation into polar lipids by TLC purification and GC analysis of FAME as described in Example 1. It was observed that ARA from the oil had incorporated poorly into the polar lipid fraction from the cells, at a level of up to only 0.6% of the total fatty acid content. This may have been due to poor mixing of the ARA oil in the medium, rendering much of the oil unavailable to the cells, or to a lack of secreted lipase activity from the Y. lipolytica cells. While incorporation of ARA from the oil was poor in these small scale cultures (20 ml), much greater incorporation was observed in larger scale cultures in fermenters where mixing was much better with agitation and aeration (see Example 6). In an initial attempt to improve ARA availability, the inventors hydrolysed some of the the oil to convert its TAG into free fatty acids, as follows.

### Experiment 1

Preparation of Fatty Acid Substrate from ARA Oil by Hydrolysis of TAG

Two similar methods were tested to hydrolyse the TAG in the ARA-rich oil, both using KOH. Method 1 was based on Lipid Analysis book, 2^{nd} edition, Christie. In this method, 0.5 g of the ARA-rich oil was mixed with 1.5 ml 1 M KOH in 95% ethanol for 1 h in a glass tube (A). After cooling the solution, 1 ml water and 1 ml hexane were added to the mixture and vortexed for 5 min. After centrifugation at 1,700 g for 5 min, the upper, hexane phase was transferred to a glass tube (B). To further extract fatty acid, 1 ml hexane was added to the lower phase, vortexed for 5 min, centrifuged for 5 min and the upper phase removed and added to tube B. The solvent from tube B was evaporated under a flow of nitrogen and the dried extract was dissolved in 0.3 ml chloroform. Method 2, based on Salimon 2011, was identical to method 1 except that 0.5 g ARA-rich oil was treated with 1.5 ml 1.75 M KOH in 90% ethanol for 1 h at 65° C. The fatty acids were extracted into hexane as in method 1. Again, the hexane was evaporated under a flow of nitrogen and the dried lipid dissolved in 0.3 ml chloroform. In both methods, the alkali was not neutralised before the hexane extraction, but this was done for later preparations of hydrolysates. However, in this experiment, the hydrolysed fatty acids were isolated by TLC and recovered, so not requiring neutralisation.

To determine the extent of TAG hydrolysis, 10 μl aliquots of the fatty acid preparations were chromatographed on TLC plates (Silica 60, Merck) using hexane/diethylether/acetic acid (70/30/1; v/v/v) as the solvent system as described in Example 1. Both methods provided efficient hydrolysis of the ARA-oil as shown by the presence of bands corresponding to FFA and the absence of bands for TAG on the TLC plate. The fatty acid composition of the hydrolysates and the fatty acids purified by TLC were almost the same as the starting ARA oil, having 47-51% ARA in the total fatty acid content of the preparations.

Culturing of Y. lipolytica with Added Fatty Acids in the Medium

The hydrolysate mix and the free fatty acid preparations were added separately to 20 ml YPD base medium for culturing Y. lipolytica strain W29, to assess the incorporation of the added fatty acids into polar lipids. The cultures also contained 1% NP40 and had a starting OD600 of 0.1. The cultures were incubated for 2 h at 28° C. after which some of the hydrolysate or free fatty acid preparation in ethanol was added to a final concentration of either 1 or 2 mg/ml culture. A control culture had added ethanol but no fatty acids. Aliquots of 4 ml culture were removed after 1, 2 and 3 days of incubation and the cells harvested by centrifugation at 3,400 g for 10 min. The supernatant was removed and each cell pellet was washed twice with water by resuspension and centrifugation. The harvested cells were then freeze-dried and the dry cell weight (DCW) measured, after which lipids were extracted and analysed as described in Example 1. The polar lipid and TAG fractions were analysed by GC of FAME. The results are presented in Table 12 for polar lipid and Table 13 for the TAG fraction from each culture.

The cell densities reached an OD600 of between 15 and 39 at day 3, with slightly higher cell densities in the cultures fed the 2 mg/ml of fatty acids, suggesting that the Y. lipolytica cells may have used some of the added fatty acids as a carbon source for growth. The GC analysis of the extracted polar lipids showed the presence of ARA up to 12.3% of the total fatty acid content incorporated at 2 days when 2 mg/ml fatty acids was added. It was observed that the ARA level in the polar lipids decreased after day 1 or 2, indicating that the fatty acid added to the medium was either being consumed or being catabolised by the cells. However, with the continued growth of the cultures, the greatest yield of ARA was observed on day 3 from the culture fed with 2 mg/ml hydrolysate, which was 20.4 mg per 100 ml.

The fatty acid composition of TAG extracted from the cells showed similar levels of ARA incorporation as for the polar lipid, up to 15% ARA of the total fatty acid content. As for the polar lipid, the ARA level generally decreased during the time course. The inventors concluded that the hydrolysis of the ARA provided for greater incorporation of the ω6 fatty acids through increased availability to the microbial cells.

TABLE 12
Fatty acid composition of ARA oil and hydrolysis products thereof, and of polar lipid from Y. lipolytica cells supplied
with the FFA preparation in the growth medium at 1 or 2 mg/ml or not supplied with the FFA preparation (Control).

| | ARA | Hydrolysate | FFA | Control (no ARA) | | | 1 mg/ml ARA FFA | | | 2 mg/ml ARA FFA | | |
|---|---|---|---|---|---|---|---|---|---|---|---|---|
| Fatty acid | oil | preparation | prep | Day 1 | Day 2 | Day 3 | Day 1 | Day 2 | Day 3 | Day 1 | Day 2 | Day 3 |
| C12:0 | 0.0 | 0.0 | 0.0 | 0.0 | 0.0 | 0.0 | 0.0 | 0.0 | 0.0 | 0.0 | 0.1 | 0.0 |
| C14:0 | 0.3 | 0.2 | 0.3 | 0.1 | 0.1 | 0.1 | 0.1 | 0.1 | 0.1 | 0.1 | 0.2 | 0.1 |
| C15:0 | 0.1 | 0.1 | 0.1 | 0.6 | 0.5 | 0.4 | 1.0 | 0.8 | 0.6 | 0.9 | 1.2 | 0.8 |
| C16:0 | 9.5 | 8.5 | 9.3 | 9.3 | 7.7 | 7.1 | 15.8 | 12.5 | 12.7 | 15.3 | 15.7 | 12.5 |
| C16:1Δ7 | 0.0 | 0.0 | 0.0 | 0.9 | 1.9 | 2.6 | 0.7 | 1.5 | 2.8 | 0.8 | 0.6 | 1.9 |
| C16:1Δ9 | 0.3 | 0.3 | 0.3 | 12.1 | 13.8 | 14.5 | 4.4 | 5.7 | 7.7 | 4.1 | 4.3 | 5.8 |
| C18:0 | 9.4 | 9.7 | 9.8 | 0.4 | 0.3 | 0.4 | 0.9 | 0.9 | 1.3 | 0.7 | 1.0 | 0.9 |
| C18:1Δ9 | 10.9 | 11.2 | 10.9 | 58.4 | 60.6 | 60.3 | 33.8 | 42.8 | 45.2 | 31.5 | 30.2 | 40.9 |
| C18:1Δ11 | 1.0 | 0.9 | 1.2 | 0.9 | 1.0 | 1.0 | 0.9 | 0.8 | 0.6 | 0.9 | 0.9 | 0.7 |
| C18:2 (LA) | 7.3 | 7.2 | 7.1 | 16.6 | 13.7 | 12.8 | 23.4 | 24.1 | 22.0 | 26.7 | 24.8 | 24.9 |
| C18:3ω6 (GLA) | 2.8 | 2.7 | 2.7 | 0.0 | 0.0 | 0.0 | 4.9 | 3.4 | 1.9 | 5.5 | 5.1 | 3.3 |
| C18:3ω3 (ALA) | 0.1 | 0.1 | 0.1 | 0.0 | 0.0 | 0.0 | 0.5 | 0.5 | 0.3 | 0.8 | 0.5 | 0.6 |
| C20:0 | 0.7 | 0.8 | 0.8 | 0.0 | 0.0 | 0.0 | 0.0 | 0.0 | 0.0 | 0.0 | 0.0 | 0.0 |
| C20:1Δ11 | 0.5 | 0.5 | 0.5 | 0.2 | 0.2 | 0.3 | 0.1 | 0.1 | 0.1 | 0.1 | 0.2 | 0.1 |
| C20:2ω6 | 0.8 | 0.8 | 0.8 | 0.0 | 0.0 | 0.0 | 0.3 | 0.1 | 0.1 | 0.3 | 0.4 | 0.1 |
| C20:3ω6 (DGLA) | 2.6 | 2.5 | 2.5 | 0.0 | 0.0 | 0.0 | 1.4 | 0.9 | 0.6 | 1.2 | 1.8 | 0.9 |
| C20:4ω6 (ARA) | 50.5 | 50.8 | 49.7 | 0.0 | 0.0 | 0.0 | 11.0 | 5.1 | 3.5 | 10.5 | 12.3 | 5.8 |
| C20:3n3 | 0.0 | 0.0 | 0.0 | 0.0 | 0.0 | 0.0 | 0.5 | 0.4 | 0.2 | 0.5 | 0.6 | 0.3 |
| C22:0 | 1.7 | 2.1 | 2.1 | 0.0 | 0.0 | 0.0 | 0.0 | 0.0 | 0.0 | 0.0 | 0.0 | 0.0 |

| | ARA | Hydrolysate | FFA | | | | | | | | | |
|---|---|---|---|---|---|---|---|---|---|---|---|---|
| Fatty acid | oil | preparation | prep | Control (no ARA) | | | 1 mg/ml ARA FFA | | | 2 mg/ml ARA FFA | | |
| C20:5ω3 (EPA) | 0.1 | 0.1 | 0.1 | 0.0 | 0.0 | 0.0 | 0.0 | 0.0 | 0.0 | 0.0 | 0.0 | 0.0 |
| C22:4ω6 (DTA) | 0.2 | 0.2 | 0.2 | 0.0 | 0.0 | 0.0 | 0.0 | 0.0 | 0.0 | 0.0 | 0.0 | 0.0 |
| C24:0 | 1.2 | 1.4 | 1.4 | 0.2 | 0.2 | 0.5 | 0.2 | 0.2 | 0.3 | 0.1 | 0.2 | 0.3 |
| Polar lipid yield | | | | 2.3 | 2.1 | 1.5 | 3.0 | 2.2 | 1.8 | 3.3 | 1.0 | 2.1 |
| (% DCW) | | | | | | | | | | | | |

TABLE 13
Fatty acid composition of TAG extracted from Y. lipolytica cells supplied or not
supplied (control) with the the ARA-containing FFA preparation in the growth medium.

| | Control (no ARA) | | | 1 mg/ml ARA FFA | | | 2 mg/ml ARA FFA | | |
|---|---|---|---|---|---|---|---|---|---|
| Fatty acid | Day 1 | Day 2 | Day 3 | Day 1 | Day 2 | Day 3 | Day 1 | Day 2 | Day 3 |
| C12:0 | 0.0 | 0.0 | 0.0 | 0.3 | 0.5 | 0.4 | 0.2 | 1.0 | 0.2 |
| C14:0 | 0.2 | 0.2 | 0.2 | 0.4 | 0.4 | 0.2 | 0.2 | 0.3 | 0.1 |
| C15:0 | 0.5 | 0.5 | 0.4 | 1.0 | 1.8 | 1.2 | 0.6 | 1.3 | 0.7 |
| C16:0 | 12.7 | 13.5 | 13.8 | 13.9 | 16.8 | 18.2 | 11.5 | 15.1 | 16.7 |
| C16:1Δ7 | 0.5 | 1.0 | 1.4 | 0.6 | 0.9 | 1.9 | 0.6 | 0.5 | 1.2 |
| C16:1Δ9 | 9.0 | 9.8 | 10.9 | 4.1 | 4.0 | 5.3 | 3.7 | 3.0 | 3.9 |
| C18:0 | 9.7 | 9.1 | 9.8 | 9.9 | 14.3 | 12.9 | 8.3 | 7.7 | 13.8 |
| C18:1Δ9 | 46.0 | 47.8 | 47.4 | 28.6 | 32.0 | 35.3 | 27.2 | 22.5 | 30.5 |
| C18:1Δ11 | 1.3 | 1.3 | 1.3 | 1.4 | 1.0 | 0.8 | 1.5 | 1.6 | 1.0 |
| C18:2 (LA) | 11.3 | 9.1 | 7.5 | 14.8 | 11.3 | 10.4 | 18.5 | 18.2 | 14.0 |
| C18:3ω6 (GLA) | 0.0 | 0.0 | 0.0 | 4.2 | 2.3 | 1.5 | 6.2 | 4.8 | 2.9 |
| C18:3ω3 (ALA) | 0.0 | 0.0 | 0.0 | 0.3 | 0.2 | 0.1 | 0.7 | 0.4 | 0.3 |
| C20:0 | 0.6 | 0.5 | 0.6 | 0.4 | 0.0 | 0.0 | 0.4 | 0.0 | 0.0 |
| C20:1Δ11 | 0.4 | 0.3 | 0.3 | 0.3 | 0.2 | 0.2 | 0.3 | 0.3 | 0.2 |
| C20:2ω6 | 0.0 | 0.0 | 0.0 | 0.5 | 0.3 | 0.2 | 0.5 | 0.6 | 0.2 |
| C20:3ω6 (DGLA) | 0.0 | 0.0 | 0.0 | 1.8 | 1.3 | 0.8 | 1.7 | 2.6 | 1.3 |
| C20:4ω6 (ARA) | 0.0 | 0.0 | 0.0 | 11.3 | 3.6 | 3.8 | 13.4 | 15.1 | 6.2 |
| C20:3n3 | 0.0 | 0.0 | 0.0 | 0.8 | 0.5 | 0.3 | 0.8 | 1.0 | 0.5 |
| C22:0 | 0.7 | 0.6 | 0.6 | 0.5 | 0.8 | 0.7 | 0.4 | 0.4 | 0.7 |
| C20:5ω3 (EPA) | 0.0 | 0.0 | 0.0 | 0.0 | 0.0 | 0.0 | 0.0 | 0.0 | 0.0 |
| C22:4ω6 (DTA) | 0.0 | 0.0 | 0.0 | 0.0 | 0.0 | 0.0 | 0.0 | 0.0 | 0.0 |
| C24:0 | 6.9 | 6.4 | 5.7 | 4.8 | 7.8 | 5.7 | 3.2 | 3.5 | 5.5 |
| TAG yield (% | 0.2 | 0.4 | 0.6 | 0.9 | 0.9 | 0.9 | 1.1 | 0.3 | 1.0 |
| DCW) | | | | | | | | | |

Further cultures were grown in the same manner, either including or excluding the tergitol in the culture medium, to understand the impact of using the tergitol on the ARA incorporation into polar lipid. On this occasion, the culture medium was supplemented with 2 mg/ml of the fatty acids or the ARA oil hydrolysate, with or without the NP40. Lipids were extracted from cells harvested on day 1 and day 2 and fractionated to provide the polar lipids. Overall, there were no significant difference observed for either the ARA content or the polar lipid yield when cultures were grown with or without tergitol.

### Experiment 2

A second experiment was carried out to extend the observations to other microbial species and over a period of 6 days to understand better the correlation between fatty acid composition and total ω6 containing polar lipid yield over time. This experiment required larger quantities of ARA-oil hydrolysate for feeding to the microbial cultures. To prepare this, ARA oil was hydrolysed using a scaled up version of method 2 (above). Briefly, 50 ml of the ARA oil was mixed with 150 ml 1.75 M KOH in 90% ethanol in a 1 L bottle and incubated at 65° C. for 2 h. The solution was vigorously mixed for 5 min every 30 min using a magnetic stirrer. Aliquots of 10 μl were applied to a TLC plate and chromatographed using a mixture of hexane/diethylether/acetic acid (70/30/1; v/v/v). GC analysis revealed that about 81% of the TAG molecules had been hydrolysed to FFA and glycerol, the lower efficiency possibly being due to inefficient mixing. The hydrolysate was neutralised with HCl and the treated lipid extracted into hexane and recovered. The fatty acid composition of the FFA and TAG fractions of the hydrolysate showed 44.2% and 40.6% ARA, respectively.

The microbes Y. lipolytica strain W29, S. cerevisiae strains INVSc1 and D5A and E. coli strains DH5α and BL21 were cultured in the presence or absence of the ARA oil hydrolysate. The Y. lipolytica and S. cerevisiae cultures were inoculated into 193 ml YPD medium at an initial OD600 of 0.1 and incubated at 28° C. with shaking at 200 rpm in 1 L bottles. The E. coli cultures were inoculated into 193 ml of LB medium and incubated at 37° C. with shaking at 250 rpm. After 2h of incubation, 6.7 ml of ARA-containing hydrolysate was added to each culture to a final concentration of 4 mg/ml culture. The pH of each culture was adjusted to 7.0 by adding HCl and incubation continued. For control cultures lacking the fatty acid supplementation, 4.34 ml of 1.75 M KOH in 90% ethanol was added and the pH adjusted to 7.0. Samples of 10 ml were removed after 16 h and daily to 6 days and the cells harvested by centrifugation at 3,400 g for 10 min. The cell pellets were washed twice with water and freeze dried to determine the dry cell weight. Lipid was extracted and analysed as before to determine the yield and fatty acid composition of polar lipid and TAG fractions, in order to determine the extent of incorporation of ARA into the polar lipid and TAG for the 6-day time course.

The data are presented in Tables 14-18. For all three species of microbes, the extent of ARA incorporation and TAG content increased steadily over the 6-day time course. The two E. coli strains exhibited the lowest levels of incorporation of ARA into polar lipid, with 1.6% and 0.1% ARA, respectively being the highest content achieved at day 5. Although the total ARA composition slowly increased over time, the polar lipid content generally decreased. It was concluded that these E. coli strains were not efficient at incorporating PUFA having C20 or C22 into polar lipids. For S. cerevisiae strain INVSc1, the same trend was observed with the highest yield of ARA-PL being at day 5 with 7.1% ARA and a total polar lipid content of 0.7%. In Y. lipolytica, both the ARA content and total polar lipid yield generally trended downwards, with the highest ARA content being achieved at 16 hours of growth with 4.9% ARA and a polar lipid content of 2.4%. However, the S. cerevisiae strain D5A maintained a steady polar lipid content of approximately 1.0% through the time course, while continuing to accumulate ARA until day 6 which peaked at 5.0%.

Considering the results from ARA incorporation, lipid yields and biomass production, the highest yield production was obtained with S. cerevisiae D5A, followed by INVSc1 and Y. lipolytica strain W29. Even though the culture conditions and the ARA incorporation extent were not considered to be optimal, it was determined that it would be useful to perform larger scale culturing in a fermenter to produce larger quantities of polar lipid incorporating the ω6 fatty acids in order to test their properties.

### Experiment 3. Analysis of the Phospholipid Classes in the Polar Lipid Extracts

To determine the level of ARA incorporation into different phospholipid classes, the polar lipid was extracted from a 3 L culture of Y. lipolytica cells that had been fed with a final concentration of 0.5 mg/ml ARA for 48 h. The polar lipid was fractionated from extracted lipid by TLC as described in Example 1 using a solvent mixture of chloroform/methanol/acetic acid/water (90/15/10/3; v/v/v/v). Lipid bands were visualized on the TLC plates by spraying with a 0.002% primuline solution in 80% acetone/water and viewing under UV light. The different lipid bands were identified by comparison with reference phospholipid standards, namely PC, PE, PS, PI, PG, PA and LPC (Avanti Polar lipids Inc, USA) in adjacent lanes on the same TLC plate. The lipid bands were collected into glass vials, mixed with a known amount of triheptadecanoin and incubated in 0.7 ml 1 N HCl/methanol (Sigma) at 80° C. for 2 h to prepare FAME from each lipid class. These were recovered and quantitated by GC to determine the amount and fatty acid composition of each PL class.

The data are presented in Table 19. Abbreviations: PC, phosphatidylcholine; PE, phosphatidylethanolamine; PI, phosphatidylinositol; PS, phosphatidylserine; PA, phosphatidic acid; PG, phosphatidylglycerol; LPC, lysophosphatidylcholine; Car, cardiolipin. PC and PE were the main phospholipids in Y. lipolytica and together constituted about 80% of the phospholipids, with about 40% each, with lower amounts of PI and PS, which constituted 9.0% and 5.6% respectively of the total PL. Other minor PLs that were observed in Y. lipolytica were PA, PG, LPC and cardiolipin (Car). ARA was incorporated into all of the analysed PL classes. The PC, PE and PA classes had levels of ARA at 19.3%, 14.0% and 18.1%, respectively, of their total fatty acid content. Lower levels of ARA incorporation were observed in the PI, PS, PG, LPC and Car classes. From Example 2, animal PI and PE have higher levels of ARA than PC, which is the major phospholipid in animal meat (Example 2).

TABLE 14
Fatty acid composition of polar lipid extracted from Y. lipolytica
strain W29 supplied or not supplied with ARA hydrolysate.

| | Non-fed (control) | | | | | | | Fed ARA-containing hydrolysate | | | | | | |
|---|---|---|---|---|---|---|---|---|---|---|---|---|---|---|
| Fatty acid | 20 h | 1 d | 2 d | 3 d | 4 d | 5 d | 6 d | 16 h | 1 d | 2 d | 3 d | 4 d | 5 d | 6 d |
| C12:0 | 0.1 | 0.0 | 0.0 | 0.0 | 0.0 | 0.0 | 0.0 | 0.0 | 0.0 | 0.0 | 0.0 | 0.0 | 0.1 | 0.0 |
| C12:1 | 0.1 | 0.0 | 0.0 | 0.0 | 0.0 | 0.0 | 0.0 | 0.1 | 0.4 | 0.1 | 0.2 | 0.5 | 0.3 | 0.2 |
| C14:0 | 0.2 | 0.1 | 0.1 | 0.1 | 0.1 | 0.1 | 0.1 | 0.2 | 0.2 | 0.1 | 0.1 | 0.1 | 0.1 | 0.1 |
| C15:0 | 0.4 | 0.3 | 0.3 | 0.3 | 0.2 | 0.3 | 0.3 | 0.4 | 0.5 | 0.4 | 0.3 | 0.3 | 0.4 | 0.4 |
| C16:0 | 12.6 | 10.9 | 9.2 | 8.0 | 7.4 | 7.2 | 7.0 | 16.5 | 16.1 | 15.2 | 12.4 | 12.1 | 13.0 | 12.4 |
| C16:1Δ7 | 1.9 | 2.1 | 2.9 | 3.1 | 3.2 | 3.5 | 4.3 | 0.8 | 1.0 | 1.2 | 1.4 | 1.5 | 1.6 | 1.9 |
| C16:1Δ9 | 8.7 | 9.0 | 9.0 | 8.1 | 7.6 | 8.0 | 8.4 | 3.5 | 3.3 | 3.0 | 3.2 | 3.3 | 3.6 | 4.1 |
| C17:1 | 1.2 | 1.3 | 2.6 | 3.8 | 4.5 | 4.8 | 4.6 | 0.6 | 0.7 | 0.9 | 1.4 | 1.9 | 2.3 | 2.4 |
| C18:0 | 0.7 | 0.4 | 0.3 | 0.2 | 0.2 | 0.3 | 0.2 | 0.5 | 0.6 | 0.8 | 0.7 | 0.7 | 0.8 | 0.8 |
| C18:1Δ9 (oleic) | 58.1 | 60.1 | 63.7 | 65.1 | 65.8 | 64.2 | 63.4 | 23.0 | 23.6 | 27.0 | 31.8 | 34.6 | 36.5 | 39.0 |
| C18:1Δ11 | 0.6 | 0.6 | 0.5 | 0.0 | 0.0 | 0.0 | 0.5 | 1.3 | 0.8 | 0.8 | 0.7 | 0.6 | 0.6 | 0.6 |
| C18:2ω6 (LA) | 15.0 | 14.8 | 11.1 | 10.9 | 10.5 | 11.0 | 10.9 | 42.0 | 40.6 | 42.2 | 42.4 | 39.7 | 35.8 | 32.0 |
| C18:3ω6 (GLA) | 0.0 | 0.0 | 0.0 | 0.0 | 0.0 | 0.0 | 0.0 | 4.0 | 4.4 | 3.3 | 2.3 | 1.7 | 1.5 | 1.9 |
| C18:3ω3 (ALA) | 0.0 | 0.0 | 0.0 | 0.0 | 0.0 | 0.0 | 0.0 | 1.6 | 1.7 | 1.7 | 1.5 | 1.3 | 1.1 | 1.0 |
| C20:1Δ11 | 0.2 | 0.2 | 0.1 | 0.1 | 0.1 | 0.1 | 0.1 | 0.0 | 0.0 | 0.0 | 0.0 | 0.0 | 0.0 | 0.0 |
| C20:2ω6 | 0.0 | 0.0 | 0.0 | 0.0 | 0.0 | 0.0 | 0.0 | 0.1 | 0.1 | 0.1 | 0.0 | 0.1 | 0.1 | 0.1 |
| C20:3ω6 (DGLA) | 0.0 | 0.0 | 0.0 | 0.0 | 0.0 | 0.0 | 0.0 | 0.4 | 0.5 | 0.4 | 0.2 | 0.2 | 0.2 | 0.3 |
| C20:4ω6 (ARA) | 0.0 | 0.0 | 0.0 | 0.0 | 0.0 | 0.0 | 0.0 | 4.9 | 5.5 | 2.8 | 1.3 | 1.2 | 1.5 | 2.4 |
| C24:0 | 0.3 | 0.2 | 0.1 | 0.2 | 0.3 | 0.3 | 0.2 | 0.1 | 0.1 | 0.2 | 0.2 | 0.3 | 0.4 | 0.3 |
| Polar lipid yield | 0.4 | 2.3 | 3.2 | 3.1 | 4.0 | 3.2 | 2.3 | 2.4 | 1.6 | 1.8 | 1.9 | 1.7 | 1.3 | 1.8 |

TABLE 15
Fatty acid composition of polar lipid extracted from S. cerevisiae
INVSc1 supplied or not supplied with ARA hydrolysate.

| | Non-fed (control) | | | | | | | Fed ARA-containing hydrolysate | | | | | | |
|---|---|---|---|---|---|---|---|---|---|---|---|---|---|---|
| Fatty acid | 20 h | 1 d | 2 d | 3 d | 4 d | 5 d | 6 d | 16 h | 1 d | 2 d | 3 d | 4 d | 5 d | 6 d |
| C12:0 | 0.5 | 0.3 | 0.2 | 0.1 | 0.1 | 0.1 | 0.1 | 0.3 | 0.1 | 0.1 | 0.2 | 0.0 | 0.1 | 0.0 |
| C14:0 | 1.0 | 0.6 | 0.3 | 0.3 | 0.4 | 0.4 | 0.4 | 0.5 | 0.4 | 0.4 | 0.2 | 0.1 | 0.1 | 0.0 |
| C15:0 | 0.2 | 0.3 | 0.6 | 0.4 | 0.3 | 0.3 | 0.2 | 1.1 | 0.9 | 0.9 | 1.2 | 0.4 | 0.4 | 0.0 |
| C16:0 | 15.4 | 15.6 | 10.6 | 11.8 | 13.4 | 14.9 | 15.2 | 32.6 | 29.6 | 28.3 | 32.0 | 25.0 | 23.7 | 22.1 |
| C16:1Δ9 | 51.4 | 48.8 | 47.0 | 44.8 | 44.3 | 43.4 | 43.6 | 7.4 | 7.8 | 7.2 | 4.0 | 3.8 | 3.9 | 4.0 |
| C18:0 | 3.0 | 4.0 | 5.7 | 6.1 | 6.1 | 7.3 | 7.7 | 4.9 | 4.6 | 4.5 | 9.3 | 7.8 | 8.1 | 10.7 |
| C18:1Δ9 (oleic) | 27.6 | 29.0 | 33.4 | 34.9 | 33.8 | 32.1 | 31.2 | 22.6 | 21.8 | 22.2 | 23.8 | 27.8 | 28.7 | 30.0 |
| C18:1Δ11 | 1.0 | 1.3 | 2.0 | 1.7 | 1.6 | 1.5 | 1.6 | 0.9 | 0.6 | 0.6 | 0.6 | 0.9 | 1.0 | 1.1 |
| C18:2ω6 (LA) | 0.0 | 0.0 | 0.0 | 0.0 | 0.0 | 0.0 | 0.0 | 21.6 | 21.1 | 23.9 | 18.7 | 20.8 | 19.6 | 17.5 |
| C18:3ω6 (GLA) | 0.0 | 0.0 | 0.0 | 0.0 | 0.0 | 0.0 | 0.0 | 6.8 | 10.5 | 9.0 | 6.0 | 6.6 | 6.5 | 6.3 |
| C18:3ω3 (ALA) | 0.0 | 0.0 | 0.0 | 0.0 | 0.0 | 0.0 | 0.0 | 0.6 | 0.7 | 0.7 | 0.4 | 0.4 | 0.4 | 0.4 |
| C20:2ω6 | 0.0 | 0.0 | 0.0 | 0.0 | 0.0 | 0.0 | 0.0 | 0.0 | 0.0 | 0.0 | 0.0 | 0.0 | 0.0 | 0.0 |
| C20:3ω6 (DGLA) | 0.0 | 0.0 | 0.0 | 0.0 | 0.0 | 0.0 | 0.0 | 0.0 | 0.2 | 0.2 | 0.0 | 0.4 | 0.5 | 0.5 |
| C20:4ω6 (ARA) | 0.0 | 0.0 | 0.0 | 0.0 | 0.0 | 0.0 | 0.0 | 0.8 | 1.7 | 2.3 | 3.5 | 5.8 | 7.1 | 7.4 |
| Polar lipid | 1.0 | 0.9 | 0.8 | 0.7 | 0.6 | 0.5 | 0.6 | 0.8 | 0.6 | 0.9 | 0.1 | 0.6 | 0.7 | 0.3 |
| yield (% DCW) | | | | | | | | | | | | | | |

TABLE 16
Fatty acid composition of polar lipid extracted from S. cerevisiae
D5A supplied or not supplied with ARA hydrolysate.

| | Non-fed (control) | | | | | | | Fed ARA-containing hydrolysate | | | | | | |
|---|---|---|---|---|---|---|---|---|---|---|---|---|---|---|
| Fatty acid | 20 h | 1 d | 2 d | 3 d | 4 d | 5 d | 6 d | 16 h | 1 d | 2 d | 3 d | 4 d | 5 d | 6 d |
| C12:0 | 0.2 | 0.3 | 0.3 | 0.2 | 0.2 | 0.1 | 0.1 | 0.1 | 0.1 | 0.1 | 0.0 | 0.0 | 0.0 | 0.0 |
| C14:0 | 1.1 | 1.1 | 1.0 | 0.9 | 0.8 | 0.7 | 0.6 | 0.6 | 0.5 | 0.2 | 0.1 | 0.1 | 0.1 | 0.1 |
| C15:0 | 0.2 | 0.2 | 0.3 | 0.2 | 0.2 | 0.2 | 0.2 | 0.5 | 0.7 | 0.4 | 0.3 | 0.3 | 0.3 | 0.3 |
| C16:0 | 18.3 | 17.8 | 17.3 | 17.8 | 18.7 | 19.3 | 19.5 | 27.0 | 31.8 | 27.3 | 22.8 | 20.1 | 17.9 | 15.5 |
| C16:1Δ9 | 48.0 | 46.9 | 46.7 | 45.2 | 42.2 | 40.3 | 38.6 | 14.6 | 17.5 | 10.1 | 9.1 | 9.1 | 11.0 | 12.3 |
| C18:0 | 2.7 | 2.8 | 3.4 | 4.0 | 5.2 | 6.2 | 7.0 | 4.4 | 4.5 | 5.3 | 6.4 | 6.7 | 6.4 | 6.4 |
| C18:1Δ9 (oleic) | 28.5 | 29.7 | 29.8 | 30.3 | 31.0 | 31.5 | 32.2 | 25.4 | 24.2 | 33.3 | 34.5 | 35.4 | 35.8 | 37.0 |
| C18:1Δ11 | 1.0 | 1.2 | 1.3 | 1.4 | 1.6 | 1.7 | 1.8 | 0.8 | 0.8 | 0.5 | 0.7 | 0.8 | 0.8 | 1.0 |
| C18:2ω6 (LA) | 0.0 | 0.0 | 0.0 | 0.0 | 0.0 | 0.0 | 0.0 | 17.3 | 12.8 | 18.0 | 19.0 | 19.1 | 17.8 | 16.2 |
| C18:3ω6 (GLA) | 0.0 | 0.0 | 0.0 | 0.0 | 0.0 | 0.0 | 0.0 | 7.1 | 5.2 | 3.6 | 5.3 | 6.0 | 5.9 | 5.4 |
| C18:3ω3 (ALA) | 0.0 | 0.0 | 0.0 | 0.0 | 0.0 | 0.0 | 0.0 | 0.5 | 0.4 | 0.4 | 0.4 | 0.4 | 0.3 | 0.3 |
| C20:2ω6 | 0.0 | 0.0 | 0.0 | 0.0 | 0.0 | 0.0 | 0.0 | 0.0 | 0.0 | 0.0 | 0.0 | 0.0 | 0.0 | 0.0 |
| C20:3ω6 (DGLA) | 0.0 | 0.0 | 0.0 | 0.0 | 0.0 | 0.0 | 0.0 | 0.2 | 0.2 | 0.1 | 0.2 | 0.2 | 0.4 | 0.5 |
| C20:4ω6 (ARA) | 0.0 | 0.0 | 0.0 | 0.0 | 0.0 | 0.0 | 0.0 | 1.5 | 1.4 | 0.7 | 1.2 | 1.9 | 3.2 | 5.0 |
| Polar lipid | 0.9 | 0.9 | 0.9 | 0.9 | 1.0 | 1.1 | 1.4 | 1.0 | 0.6 | 1.1 | 1.1 | 1.1 | 0.9 | 0.9 |
| yield (% DCW) | | | | | | | | | | | | | | |

TABLE 17
Fatty acid composition of polar lipid extracted from E. coli
DH5α supplied or not supplied with ARA hydrolysate.

| | Non-fed (control) | | | | | | | Fed ARA-containing hydrolysate | | | | | | |
|---|---|---|---|---|---|---|---|---|---|---|---|---|---|---|
| Fatty acid | 20 h | 1 d | 2 d | 3 d | 4 d | 5 d | 6 d | 16 h | 1 d | 2 d | 3 d | 4 d | 5 d | 6 d |
| C12:0 | 0.5 | 0.4 | 0.5 | 0.5 | 0.4 | 0.3 | 0.5 | 0.1 | 0.1 | 0.1 | 0.1 | 0.1 | 0.1 | 0.2 |
| C14:0 | 4.8 | 5.0 | 5.5 | 5.7 | 5.4 | 5.2 | 5.4 | 3.4 | 3.9 | 4.2 | 4.0 | 4.1 | 4.5 | 5.1 |
| C15:0 | 2.8 | 2.9 | 2.6 | 2.4 | 2.3 | 2.4 | 2.8 | 2.5 | 2.8 | 2.6 | 2.5 | 2.4 | 1.9 | 1.8 |
| C16:0 | 44.0 | 45.1 | 47.6 | 48.6 | 50.8 | 51.0 | 48.7 | 57.4 | 57.0 | 57.1 | 57.1 | 56.2 | 55.3 | 55.7 |
| C16:1Δ9 | 7.5 | 10.3 | 11.9 | 11.0 | 9.0 | 6.6 | 5.5 | 1.2 | 1.0 | 0.7 | 0.7 | 0.9 | 1.0 | 0.7 |
| C18:0 | 0.5 | 0.2 | 0.1 | 0.5 | 0.5 | 0.6 | 0.2 | 2.0 | 2.2 | 2.4 | 2.6 | 2.9 | 2.5 | 2.7 |
| C18:1Δ9 (oleic) | 0.0 | 0.0 | 0.0 | 0.0 | 0.0 | 0.0 | 0.0 | 1.3 | 1.8 | 2.5 | 2.5 | 2.9 | 4.0 | 4.1 |
| C18:1Δ11 | 9.8 | 9.7 | 8.1 | 7.6 | 7.6 | 7.6 | 7.5 | 1.3 | 1.2 | 1.5 | 1.4 | 1.9 | 2.0 | 1.4 |
| C19:0 | 5.5 | 5.6 | 5.1 | 5.4 | 7.6 | 8.4 | 8.9 | 6.3 | 6.3 | 5.9 | 5.7 | 5.5 | 5.5 | 5.4 |
| C17:0c | 21.4 | 18.3 | 16.7 | 16.8 | 15.1 | 16.7 | 18.9 | 21.0 | 19.3 | 17.3 | 17.1 | 16.8 | 17.2 | 16.5 |
| C19:0c | 3.2 | 2.5 | 1.9 | 1.6 | 1.3 | 1.4 | 1.6 | n.d. | n.d. | n.d. | n.d. | n.d. | n.d. | n.d. |
| C18:2ω6 (LA) | 0.0 | 0.0 | 0.0 | 0.0 | 0.0 | 0.0 | 0.0 | 0.5 | 0.3 | 0.3 | 0.3 | 0.4 | 0.4 | 0.4 |
| C18:3ω6 (GLA) | 0.0 | 0.0 | 0.0 | 0.0 | 0.0 | 0.0 | 0.0 | 1.9 | 2.9 | 4.2 | 4.7 | 4.8 | 4.3 | 4.8 |
| C20:3ω6 (DGLA) | 0.0 | 0.0 | 0.0 | 0.0 | 0.0 | 0.0 | 0.0 | 1.1 | 1.2 | 1.1 | 1.0 | 1.0 | 1.0 | 1.0 |
| C20:4ω6 (ARA) | 0.0 | 0.0 | 0.0 | 0.0 | 0.0 | 0.0 | 0.0 | 0.0 | 0.0 | 0.0 | 0.1 | 0.1 | 0.1 | 0.1 |
| Polar lipid | 1.0 | 2.8 | 2.8 | 2.3 | 2.4 | 1.1 | 1.8 | 2.6 | 2.2 | 2.5 | 2.1 | 1.7 | 1.9 | 1.3 |
| yield (% DCW) | | | | | | | | | | | | | | |

TABLE 18
Fatty acid composition of polar lipid extracted from E. coli BL21 supplied or not supplied with ARA hydrolysate.

| | Non-fed (control) | | | | | | | Fed ARA-containing hydrolysate | | | | | | |
|---|---|---|---|---|---|---|---|---|---|---|---|---|---|---|
| Fatty acid | 20 h | 1 d | 2 d | 3 d | 4 d | 5 d | 6 d | 16 h | 1 d | 2 d | 3 d | 4 d | 5 d | 6 d |
| C12:0 | 2.0 | 0.9 | 1.3 | 0.9 | 0.8 | 1.0 | 1.1 | 0.2 | 0.2 | 0.2 | 0.2 | 0.2 | 0.2 | 0.2 |
| C14:0 | 4.3 | 3.3 | 2.8 | 2.4 | 2.2 | 2.6 | 2.7 | 1.2 | 1.5 | 1.8 | 1.8 | 1.7 | 1.9 | 2.0 |
| C15:0 | 0.3 | 0.3 | 0.4 | 0.4 | 0.4 | 0.4 | 0.4 | 0.3 | 0.4 | 0.7 | 1.8 | 1.5 | 1.3 | 1.1 |
| C16:0 | 33.8 | 33.3 | 30.5 | 34.8 | 40.1 | 43.6 | 42.4 | 38.6 | 37.2 | 38.8 | 38.2 | 37.8 | 39.4 | 41.1 |
| C16:1Δ9 | 8.4 | 10.2 | 8.9 | 8.0 | 7.8 | 7.1 | 7.1 | 8.1 | 8.2 | 4.8 | 4.4 | 4.9 | 4.6 | 4.0 |
| C18:0 | 0.8 | 0.5 | 0.2 | 1.1 | 1.2 | 1.3 | 0.2 | 2.4 | 2.7 | 2.8 | 3.1 | 3.3 | 3.4 | 3.4 |
| C18:1Δ9 (oleic) | 0.0 | 0.0 | 0.0 | 0.0 | 0.0 | 0.0 | 0.0 | 1.9 | 3.1 | 8.2 | 9.7 | 8.9 | 9.9 | 11.6 |
| C18:1Δ11 | 32.1 | 34.6 | 39.0 | 35.7 | 31.0 | 25.5 | 26.3 | 37.6 | 36.3 | 29.3 | 27.1 | 29.0 | 27.1 | 24.5 |
| C19:0 | 2.8 | 3.2 | 3.4 | 3.5 | 4.7 | 5.4 | 5.7 | 1.7 | 1.8 | 2.5 | 2.3 | 2.0 | 1.9 | 1.9 |
| C17:0c | 11.6 | 10.6 | 11.1 | 11.0 | 9.9 | 11.2 | 12.1 | 5.9 | 5.4 | 7.0 | 6.6 | 5.6 | 5.2 | 5.1 |
| C19:0c | 4.0 | 3.2 | 2.3 | 2.2 | 1.9 | 1.9 | 2.0 | n.d. | n.d. | n.d. | n.d. | n.d. | n.d. | n.d. |
| C18:2ω6 (LA) | 0.0 | 0.0 | 0.0 | 0.0 | 0.0 | 0.0 | 0.0 | 1.3 | 1.7 | 1.9 | 2.0 | 2.2 | 2.1 | 2.0 |
| C18:3ω6 (GLA) | 0.0 | 0.0 | 0.0 | 0.0 | 0.0 | 0.0 | 0.0 | 0.1 | 0.2 | 0.8 | 1.1 | 0.9 | 1.0 | 1.1 |
| C20:3ω6 (DGLA) | 0.0 | 0.0 | 0.0 | 0.0 | 0.0 | 0.0 | 0.0 | 0.3 | 0.4 | 0.5 | 0.4 | 0.4 | 0.4 | 0.4 |
| C20:4ω6 (ARA) | 0.0 | 0.0 | 0.0 | 0.0 | 0.0 | 0.0 | 0.0 | 0.3 | 0.7 | 0.7 | 1.0 | 1.5 | 1.6 | 1.5 |
| Polar lipid | 1.9 | 3.1 | 2.3 | 2.1 | 2.1 | 1.7 | 1.6 | 1.6 | 1.3 | 1.6 | 1.0 | 0.8 | 0.9 | 0.9 |
| yield (% DCW) | | | | | | | | | | | | | | |

TABLE 19
Fatty acid composition and proportions of phospholipid classes in Y. lipolytica
polar lipids after culturing in the presence of 0.5 mg ARA/ml.

| | Y. lipolytica | | | | | | | | ARA-fed Y. lipolytica | | | | | | | |
|---|---|---|---|---|---|---|---|---|---|---|---|---|---|---|---|---|
| | PC | PE | PI | PS | PA | PG | LPC | Car | PC | PE | PI | PS | PA | PG | LPC | Car |
| C12:0 | 0.0 | 0.0 | 0.0 | 0.1 | 0.1 | 0.3 | 0.8 | 0.9 | 0.0 | 0.0 | 0.1 | 0.1 | 0.2 | 0.3 | 0.4 | 1.2 |
| C14:0 | 0.2 | 0.3 | 0.3 | 0.4 | 0.2 | 0.6 | 1.0 | 0.6 | 0.2 | 0.1 | 0.3 | 0.3 | 0.1 | 0.9 | 0.7 | 0.6 |
| C15:0 | 0.3 | 0.7 | 1.1 | 1.3 | 0.4 | 1.3 | 2.7 | 1.7 | 0.5 | 0.6 | 1.0 | 1.7 | 0.3 | 2.5 | 1.6 | 1.5 |
| C16:0 | 7.1 | 16.3 | 39.7 | 41.0 | 12.2 | 31.0 | 40.2 | 28.2 | 10.9 | 15.3 | 38.8 | 40.8 | 7.2 | 41.3 | 30.4 | 25.9 |
| C16:1Δ7 | 0.9 | 1.2 | 0.7 | 1.0 | 0.9 | 0.9 | 0.7 | 1.0 | 0.8 | 1.0 | 0.7 | 1.5 | 0.6 | 0.7 | 0.6 | 1.1 |
| C16:1Δ9 | 14.5 | 10.7 | 4.0 | 3.5 | 7.0 | 8.2 | 4.0 | 9.2 | 9.9 | 7.3 | 3.0 | 3.7 | 5.3 | 6.9 | 4.7 | 6.5 |
| C17:1 | 1.7 | 1.4 | 0.6 | 0.6 | 1.2 | 1.0 | 0.0 | 0.9 | 1.8 | 1.3 | 0.8 | 0.9 | 1.1 | 1.1 | 0.8 | 0.9 |
| C18:0 | 2.3 | 1.7 | 2.3 | 2.9 | 4.6 | 3.0 | 12.2 | 22.4 | 1.9 | 1.4 | 3.0 | 2.0 | 4.3 | 3.3 | 7.3 | 22.6 |
| C18:1Δ9 | 41.5 | 42.0 | 33.1 | 37.2 | 36.7 | 34.8 | 21.6 | 20.0 | 45.6 | 46.3 | 43.1 | 44.2 | 39.4 | 33.3 | 28.3 | 21.1 |
| C18:2ω6 (LA) | 31.1 | 25.6 | 18.1 | 12.0 | 36.2 | 18.9 | 16.2 | 14.8 | 7.0 | 11.4 | 6.1 | 2.8 | 21.3 | 4.1 | 7.0 | 12.9 |
| C18:3ω6 (GLA) | 0.0 | 0.0 | 0.0 | 0.0 | 0.0 | 0.0 | 0.0 | 0.0 | 1.5 | 1.1 | 0.4 | 0.4 | 1.3 | 0.5 | 0.8 | 1.2 |
| C20:0 | 0.1 | 0.1 | 0.0 | 0.0 | 0.2 | 0.0 | 0.4 | 0.2 | 0.1 | 0.0 | 0.0 | 0.1 | 0.1 | 0.0 | 0.1 | 0.3 |
| C20:1 | 0.0 | 0.1 | 0.0 | 0.0 | 0.0 | 0.1 | 0.0 | 0.0 | 0.0 | 0.0 | 0.0 | 0.1 | 0.0 | 0.2 | 0.0 | 0.0 |
| C20:3ω6 (DGLA) | 0.0 | 0.0 | 0.0 | 0.0 | 0.0 | 0.0 | 0.0 | 0.0 | 0.4 | 0.3 | 0.1 | 0.1 | 0.3 | 0.4 | 0.0 | 0.0 |
| C20:4ω6 (ARA) | 0.0 | 0.0 | 0.0 | 0.0 | 0.0 | 0.0 | 0.0 | 0.0 | 19.3 | 14.0 | 1.9 | 1.3 | 18.1 | 4.4 | 3.7 | 3.7 |
| C22:0 | 0.0 | 0.0 | 0.0 | 0.0 | 0.0 | 0.0 | 0.1 | 0.1 | 0.0 | 0.0 | 0.0 | 0.0 | 0.1 | 0.0 | 0.0 | 0.0 |
| C22:4n6 | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 |
| C24:0 | 0.0 | 0.0 | 0.0 | 0.0 | 0.2 | 0.0 | 0.0 | 0.0 | 0.0 | 0.0 | 0.7 | 0.0 | 0.1 | 0.0 | 13.5 | 0.4 |
| % of total | 36.6 | 42.3 | 9.0 | 5.6 | 3.3 | 2.5 | 0.3 | 0.4 | 43.7 | 40.0 | 9.0 | 2.9 | 2.3 | 1.2 | 0.7 | 0.2 |

### Experiment 4

To test for incorporation of different ω6 fatty acids into polar lipids, cells of the three species were cultured separately in the presence of GLA, DGLA, DPA-ω6 or ARA, or in the absence of added fatty acid. Y. lipolytica strain W29 and S. cerevisiae strain INVSc1 cells were each inoculated into 20 ml YPD medium and E. coli strain DH5α cells were inoculated into 20 ml LB medium in 100 ml bottles. The media also contained 1% tergitol (NP40). The initial cellular density was set at an OD600 of 0.1 and the yeast cultures were incubated at 28° C. with mixing at 200 rpm while E. coli was cultured at 37° C. After 2 h of incubation, the fatty acids GLA, DGLA, ARA and DPA-ω6, each of 99% purity (NuChek Inc, USA) dissolved in ethanol were added to a final concentration of 0.5 mg/ml and incubation continued. The DH5x, W29 and INVSc1 cells were harvested after 1 day, 2 days and 4 days of culturing, respectively, due to their different growth rates. The harvested cells were pelleted by centrifugation at 4,600 g for 15 min. The cell pellets were washed twice with water by resuspension and centrifugation, and the cell pellets freeze dried. Lipid extraction and analysis of both the content and fatty acid composition of extracted polar lipid and TAG was carried out as before.

Analogous cultures are produced using adrenic acid (docosatetraenoic acid, DTA, C22:4ω6) to supplement the culture medium, and polar lipids are extracted from the cells.

The data are provided in Tables 20-22. High levels of incorporation of the different ω6 fatty acids were observed in the polar lipid fraction of Y. lipolytica (Table 20). The proportion of GLA, DGLA and ARA was 47.1%, 29.4% and 20.5%, respectively, of the total fatty acid content of the polar lipid fraction extracted from those cells. S. cerevisiae exhibited even higher levels of GLA, DGLA or ARA at 60.7%, 59.6% and 50.8%, respectively, in the polar lipid fraction after 4 days of incubation (Table 21), while E. coli incorporated much lower levels of these fatty acids at 6.4%, 2.5% and 0.7%, respectively, in the polar lipid after 24 h of culturing (Table 22). The TAG fractions from the yeast cells also showed high levels of these fatty acids. The S. cerevisiae cells exhibited TAG with incorporation of 78.1%, 80.2% and 76.8% of GLA, DGLA and ARA, respectively, indicating high activity of the acyltransferases in S. cerevisiae towards these exogenous ω6 fatty acids and efficient incorporation into TAG. Polar lipid accumulation was higher, at greater than 2.0% of DCW, in W29 and DH5α, while INVSc1 contained approximately 1% polar lipid. Since E. coli was devoid of TAG, the only lipid in the extract from that species was polar lipid which could be prepared without needing fractionation to remove TAG. It was also concluded, however, that E. coli has a limited ability of incorporating exogenously fed ω6 fatty acids, evident by the low extent of incorporation.

TABLE 20
Fatty acid composition of polar lipids and TAG in Y. lipolytica strain W29 after
culturing with ω6 fatty acids. The percentages are the average of triplicate assays.

| | Polar lipids | | | | Triacylglycerol | | | |
|---|---|---|---|---|---|---|---|---|
| Fatty acid | None | GLA | DGLA | ARA | None | GLA | DGLA | ARA |
| C14:0 | 0.1 | 0.1 | 0.2 | 0.1 | 0.2 | 0.2 | 0.2 | 0.2 |
| C15:0 | 0.5 | 2.1 | 1.3 | 1.0 | 0.5 | 1.2 | 0.8 | 0.8 |
| C16:0 | 8.4 | 19.5 | 12.6 | 13.1 | 15.5 | 14.1 | 15.7 | 19.8 |
| C16:1Δ7 | 1.5 | 0.2 | 0.5 | 0.3 | 0.9 | 0.3 | 0.4 | 0.3 |
| C16:1Δ9 | 15.1 | 5.0 | 11.2 | 11.0 | 9.4 | 3.1 | 5.8 | 6.9 |
| C17:1 | 2.6 | 1.5 | 1.8 | 1. | 1.3 | 0.6 | 0.8 | 0.9 |
| C18:0 | 0.4 | 2.5 | 0.7 | 0.6 | 9.3 | 8.2 | 7.9 | 10.2 |
| C18:1Δ9 | 53.9 | 18.6 | 31.6 | 39.9 | 44.0 | 1 | 22.5 | 28.5 |
| C18:1Δ11 | 1.0 | 0.1 | 0.3 | 0.5 | 0.9 | 0.4 | 0.5 | 0.8 |
| C18:2 (LA) | 16.4 | 2.4 | 8.6 | 9.8 | 10.4 | 2.4 | 5.4 | 5.3 |
| C18:3ω6 (GLA) | 0.0 | 47.1 | 1.6 | 1.2 | 0.0 | 47.7 | 1.4 | 0.9 |
| C20:0 | 0.0 | 0.1 | 0.0 | 0.0 | 0.5 | 0.5 | 0.3 | 0.4 |
| C20:3ω6 (DGLA) | 0.0 | 0.4 | 29.4 | 0.3 | 0.0 | 0.8 | 33.4 | 0.5 |
| C20:4ω6 (ARA) | 0.0 | 0.0 | 0.0 | 20.5 | 0.0 | 0.4 | 0.0 | 16.7 |
| C22:0 | 0.0 | 0.0 | 0.0 | 0.0 | 0.6 | 0.5 | 0.3 | 0.5 |
| C24:0 | 0.2 | 0.4 | 0.2 | 0.2 | 6.4 | 4.0 | 4.6 | 7.1 |
| % of DCW | 1.9 | 2.1 | 2.4 | 2.4 | 0.4 | 0.5 | 0.8 | 0.8 |

TABLE 21
Fatty acid composition of polar lipids and TAG in S. cerevisiae strain INVSc1 after
culturing with ω6 fatty acids. The percentages are the average of triplicate assays.

| | Polar lipids | | | | Triacylglycerol | | | |
|---|---|---|---|---|---|---|---|---|
| Fatty acid | None | GLA | DGLA | ARA | None | GLA | DGLA | ARA |
| C14:0 | 0.5 | 0.3 | 0.8 | 1.3 | 1.0 | 0.5 | 1.0 | 1.3 |
| C15:0 | 0.6 | 1.1 | 1.3 | 2.6 | 0.7 | 0.6 | 0.6 | 1.1 |
| C16:0 | 13.3 | 21.8 | 21.1 | 26.8 | 14.8 | 10.0 | 8.3 | 12.9 |
| C16:1Δ9 | 47.2 | 4.8 | 8.2 | 7.7 | 47.9 | 1.6 | 3.5 | 2.5 |
| C18:0 | 5.5 | 7.8 | 5.6 | 6.4 | 6.2 | 4.6 | 2.6 | 3.6 |
| C18:1Δ9 | 31.6 | 3.2 | 2.8 | 3.3 | 28.2 | 0.8 | 1.4 | 1.0 |
| C18:1Δ11 | 1.3 | 0.1 | 0.1 | 0.1 | 1.2 | 0.0 | 0.1 | 0.0 |
| C18:2 (LA) | 0.0 | 0.2 | 0.1 | 0.2 | 0.0 | 3.5 | 2.3 | 0.1 |
| C18:3ω6 (GLA) | 0.0 | 60.7 | 0.2 | 0.5 | 0.0 | 78.1 | 0.0 | 0.5 |
| C20:3ω6 (DGLA) | 0.0 | 0.1 | 59.6 | 0.3 | 0.0 | 0.3 | 80.2 | 0.2 |
| C20:4ω6 (ARA) | 0.0 | 0.0 | 0.0 | 50.8 | 0.0 | 0.0 | 0.0 | 76.8 |
| % of DCW | 0.8 | 1.0 | 0.9 | 0.9 | 2.4 | 2.8 | 3.0 | 2.7 |

TABLE 22
Fatty acid composition of polar lipids in E. coli
strain DH5α after culturing with ω6 fatty
acids. The percentages are the average of triplicate assays.

| | Non-fed | GLA | DGLA | ARA |
|---|---|---|---|---|
| C14:0 | 4.7 | 4.2 | 3.9 | 3.8 |
| C15:0 | 6.0 | 5.1 | 9.7 | 7.0 |
| C16:0 | 46.9 | 56.9 | 52.2 | 55.1 |
| C16:1Δ7 | 10.3 | 0.8 | 0.8 | 0.8 |
| C18:0 | 0.9 | 1.9 | 1.8 | 1.7 |
| C18:1Δ9 | 0.0 | 0.1 | 0.1 | 0.2 |
| C18:1Δ11 | 10.3 | 1.4 | 1.4 | 1.4 |
| C19:0 | 4.8 | 6.4 | 6.4 | 6.5 |
| C17:0c | 14.1 | 16.2 | 16.0 | 16.2 |
| C19:0c | 2.0 | 6.6 | 6.3 | 6.5 |
| C18:3ω6 (GLA) | 0.0 | 0.3 | 0.0 | 0.1 |
| C20:3ω6 (DGLA) | 0.0 | 0.0 | 1.3 | 0.0 |
| C20:4ω6 (ARA) | 0.0 | 0.0 | 0.0 | 0.7 |
| % of DCW | 1.7 | 2.1 | 2.0 | 1.9 |

### Experiment 5

This experiment was carried out in an attempt to modify the level of PL classes in Y. lipolytica and S. cerevisiae by addition of compounds that might be incorporated into phospholipid head groups, namely, inositol, choline and ethanolamine, in the growth medium. This was also done to see whether the ratios of PL classes could be modified.

Cultures of 10 ml of Y. lipolytica or S. cerevisiae were grown in YPD medium containing 1% tergitol for 1 h at 28° C. with shaking at 250 rpm, adjusting the OD600 to 0.1 at the beginning of incubation. After 1 h, myo-inositol, choline chloride or ethanolamine were added to the cultures at the concentrations of 0.1 mM, 1 mM or 1 mM, respectively, and incubation continued for a further 24 h. Cell harvesting, lipid extraction, fractionation, and analysis was performed as mentioned in Example 1.

The results are shown in Table 23 and Table 24. Myo-inositol feeding resulted in an almost 5-fold increase of PI content (0.09%) in the cells, compared to the non-fed control (0.02%). The level of C18:0 decreased from 25.1% to 19.7%, accompanied by an increase in C18:1 from 24.5% to 28.8%. A decrease in the C16:0 content was observed in each of PC and PE, accompanied by increased content of the unsaturated fatty acids C16:1 and C18:1, compared to non-fed cells. The feeding of choline chloride did not have any significant effect on either the fatty acid composition or total content of any lipid class. Although, no significant changes were observed for PE following ethanolamine feeding, other lipid classes were affected. Importantly, the PC content was reduced by more than 30% as a result of feeding (from 0.32% to 0.22%). Ethanolamine feeding also affected the fatty acid composition of PI, with increased C16:1 accompanied by decreases in both C18:0 and C18:1. Although PG is a minor polar lipid in S. cerevisiae (0.2% DCW), the feeding of ARA with either choline chloride or ethanolamine had a significant affect on the fatty acid composition, with ARA content being 83.8% and 74.9%, respectively, compared to 5.7% with ARA feeding only.

TABLE 23
Fatty acid composition of phospholipid classes from S. cerevisiae grown
in the absence or presence of myo-inositol and ARA in the culture medium.

| | S. cerevisiae + | | | | | S. cerevisiae + | | | | | S. cerevisiae + ARA + | | | | |
|---|---|---|---|---|---|---|---|---|---|---|---|---|---|---|---|
| | 0.1 mM myo-inositol | | | | | 0.2 mM myo-inositol | | | | | 0.2 mM myo-inositol | | | | |
| | PI | PC | PE | PS | CL | PI | PC | PE | PS | CL | PI | PC | PE | PS | CL |
| C10:0 | 0.0 | 0.0 | 0.0 | 0.0 | 0.0 | 0.0 | 0.0 | 0.0 | 0.0 | 0.0 | 0.0 | 0.0 | 0.0 | 0.0 | 3.4 |
| C12:0 | 0.7 | 0.4 | 0.0 | 0.0 | 0.0 | 0.7 | 0.6 | 0.5 | 0.0 | 0.0 | 1.9 | 1.1 | 0.3 | 0.0 | 2.0 |
| C14:0 | 0.0 | 0.6 | 0.0 | 0.0 | 0.0 | 0.0 | 0.7 | 0.6 | 0.0 | 0.0 | 1.1 | 1.5 | 0.5 | 0.0 | 2.3 |
| C16:0 | 28.2 | 13.3 | 19.7 | 36.6 | 16.5 | 29.6 | 12.7 | 18.7 | 38.1 | 42.6 | 32.5 | 30.0 | 22.7 | 41.4 | 25.8 |
| C16:1Δ9 | 22.6 | 54.9 | 46.4 | 21.8 | 44.5 | 23.8 | 60.1 | 46.5 | 20.5 | 20.1 | 9.8 | 13.4 | 15.3 | 21.9 | 8.5 |
| C18:0 | 19.7 | 4.3 | 0.0 | 0.0 | 0.0 | 17.3 | 3.2 | 0.9 | 0.0 | 20.9 | 12.9 | 3.2 | 2.1 | 0.0 | 5.6 |
| C18:1Δ9 | 28.8 | 23.6 | 33.9 | 41.6 | 39.0 | 28.6 | 20.2 | 31.9 | 41.5 | 16.4 | 8.2 | 3.8 | 6.4 | 21.5 | 4.2 |
| C18:1Δ11 | 0.0 | 2.9 | 0.0 | 0.0 | 0.0 | 0.0 | 2.5 | 0.9 | 0.0 | 0.0 | 0.0 | 0.0 | 0.0 | 0.0 | 0.0 |
| C18:2 | 0.0 | 0.0 | 0.0 | 0.0 | 0.0 | 0.0 | 0.0 | 0.0 | 0.0 | 0.0 | 0.0 | 0.0 | 0.6 | 0.0 | 0.0 |
| C18:3ω6 | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 |
| C20:3ω6 | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 |
| C20:4ω6 | 0.0 | 0.0 | 0.0 | 0.0 | 0.0 | 0.0 | 0.0 | 0.0 | 0.0 | 0.0 | 33.5 | 47.0 | 52.1 | 15.2 | 48.1 |
| lipid %/DW | 0.09 | 0.34 | 0.16 | 0.04 | 0.01 | 0.15 | 0.41 | 0.23 | 0.07 | 0.03 | 0.20 | 0.53 | 0.38 | 0.08 | 0.07 |

TABLE 24
Fatty acid composition of phospholipid classes from Y. lipolytica grown
in the absence or presence of myo-inositol and ARA in the culture medium.

| | Y. lipolytica + | | | | | Y. lipolytica + | | | | | Y. lipolytica + ARA + | | | | |
|---|---|---|---|---|---|---|---|---|---|---|---|---|---|---|---|
| | 0.1 mM myo-inositol | | | | | 0.2 mM myo-inositol | | | | | 0.2 mM myo-inositol | | | | |
| | PI | PC | PE | PS | CL | PI | PC | PE | PS | CL | PI | PC | PE | PS | CL |
| C14:0 | 0.0 | 0.2 | 0.3 | 0.0 | 0.0 | 0.0 | 0.0 | 0.0 | 0.0 | 0.0 | 0.0 | 0.0 | 0.0 | 0.0 | 0.0 |
| C14:1 | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 |
| C15:0 | 1.6 | 0.6 | 0.8 | 1.7 | 0.0 | 1.3 | 0.0 | 0.5 | 0.0 | 0.0 | 2.0 | 0.6 | 1.1 | 2.3 | 0.0 |
| C16:0 | 35.7 | 7.5 | 14.3 | 36.1 | 2.2 | 36.2 | 3.1 | 10.3 | 37.6 | 14.0 | 38.9 | 6.8 | 19.0 | 38.3 | 6.0 |
| C16:1Δ9 | 5.5 | 13.6 | 11.2 | 5.8 | 4.9 | 5.9 | 14.8 | 11.3 | 6.9 | 9.6 | 4.1 | 8.3 | 7.8 | 6.5 | 5.7 |
| C17:1 | 0.0 | 1.7 | 1.5 | 0.0 | 0.9 | 0.0 | 2.0 | 1.6 | 0.0 | 0.0 | 0.0 | 0.9 | 0.9 | 0.0 | 0.0 |
| C18:0 | 1.9 | 1.3 | 1.3 | 1.8 | 0.0 | 0.0 | 0.0 | 0.4 | 0.0 | 4.2 | 0.0 | 0.5 | 0.4 | 0.0 | 1.2 |
| C18:1Δ9 | 31.6 | 35.7 | 38.4 | 34.4 | 32.4 | 43.9 | 54.8 | 53.1 | 49.1 | 39.3 | 44.5 | 38.7 | 43.1 | 49.7 | 33.0 |
| C18:1Δ11 | 0.0 | 0.7 | 0.4 | 0.0 | 0.0 | 0.0 | 0.8 | 0.5 | 0.0 | 0.0 | 0.0 | 0.3 | 0.0 | 0.0 | 0.0 |
| C18:2 | 21.7 | 38.4 | 31.8 | 20.2 | 59.6 | 12.7 | 24.5 | 22.3 | 6.5 | 32.8 | 5.1 | 7.0 | 8.5 | 3.2 | 26.2 |
| C18:3ω6 | 0.0 | 0.0 | 0.0 | 0.0 | 0.0 | 0.0 | 0.0 | 0.0 | 0.0 | 0.0 | 0.0 | 0.9 | 0.9 | 0.0 | 0.0 |
| C20:1Δ11 | 0.0 | 0.3 | 0.0 | 0.0 | 0.0 | 0.0 | 0.0 | 0.0 | 0.0 | 0.0 | 0.0 | 0.0 | 0.0 | 0.0 | 0.0 |
| C20:3ω6 | 0.0 | 0.0 | 0.0 | 0.0 | 0.0 | 0.0 | 0.0 | 0.0 | 0.0 | 0.0 | 0.0 | 0.0 | 0.0 | 0.0 | 0.0 |
| C20:4ω6 | 0.0 | 0.0 | 0.0 | 0.0 | 0.0 | 0.0 | 0.0 | 0.0 | 0.0 | 0.0 | 5.4 | 36.0 | 18.3 | 0.0 | 27.9 |
| C24:0 | 2.1 | 0.0 | 0.0 | 0.0 | 0.0 | 0.0 | 0.0 | 0.0 | 0.0 | 0.0 | 0.0 | 0.0 | 0.0 | 0.0 | 0.0 |
| lipid %/DW | 0.11 | 1.64 | 0.95 | 0.13 | 0.21 | 0.06 | 0.71 | 0.35 | 0.04 | 0.05 | 0.11 | 1.32 | 0.37 | 0.06 | 0.19 |

### Experiment 6

To prepare a larger amount of polar lipid to test in various Maillard reactions, Y. lipolytica strain W29 was cultured in the presence of ARA fatty acid (NuChek Inc. USA) in a total of 3 L of YPD medium. A second culture was prepared at a 1 L scale. ARA dissolved in ethanol was added to a final concentration of 0.5 mg/ml culture and the cells were harvested after 48 h of incubation as described in previous experiments. After freeze drying, lipids were extracted from the cells as described in Example 1. The fatty acid composition and amounts of polar lipid and TAG were determined by preparation of FAME and GC analysis as previously described.

Feeding ARA (0.5 mg/ml) to the larger volumes of cultures for 48 hours produced polar lipid with an ARA content of 14.1% or 16.3% (Table 25). Although the cultures exhibited a higher ARA content following 24 hours of growth (23.8%), the total yield of polar lipid (2 mg) was significantly lower when compared to cultures that were grown for 48 h (128 mg). The ARA-containing Y. lipolytica polar lipid was isolated from the 3 L culture and was subsequently used in various Maillard reactions to investigate the different aroma and volatile characteristics.

Blending of Polar Lipids with Other Lipids

Polar lipids extracted and purified as described in this Example 5 are mixed with an oil such as a vegetable oil, for example canola oil or soy oil, to provide blends of oils comprising the polar lipid and non-polar lipids in the ratios of 51:49, 60:40, 70:30, 80:20 and 90:10 on a weight basis. To do this, the amount of polar lipid in each preparation is determined on a weight basis, where the polar lipid preparation contains material other than lipid. The oil used such as vegetable oil contains at least 90% or 95% non-polar lipid, almost entirely TAG. The blends are considered to be useful in providing easier food ingredient formulation and processing for food production since the blends may be liquids at room temperature.

TABLE 25
Fatty acid composition of polar lipids in Y. lipolytica
strain W29 after culturing with ARA at 1 L and 3 L scale.
The yield is the total yield from 10 ml culture.

| | |
|---|---|
| 1 L Culture | 3 L Culture |

| Fatty acid | 48 h | 24 h | 48 h |
|---|---|---|---|
| C14:0 | 0.2 | 0.3 | 0.0 |
| C15:0 | 0.8 | 1.0 | 0.0 |
| C16:0 | 15.6 | 15.7 | 15.5 |
| C16:1Δ7 | 0.8 | 0.3 | 0.9 |
| C16:1Δ9 | 8.6 | 8.8 | 8.4 |
| C16:2 | 0.2 | 0.3 | 0.6 |
| C18:0 | 1.4 | 1.0 | 1.7 |
| C18:1Δ9 | 46.9 | 30.4 | 44.8 |
| C18:1Δ11 | 0.4 | 0.4 | 0.4 |
| C18:2ω6 (LA) | 9.1 | 15.0 | 9.6 |
| C18:3ω6 (GLA) | 1.4 | 1.1 | 1.2 |
| C18:3ω3 (ALA) | 0.0 | 0.0 | 0.0 |
| C20:0 | 0.0 | 0.0 | 0.0 |
| C20:1Δ11 | 0.0 | 0.2 | 0.0 |
| C20:2ω6 (EDA) | 0.0 | 0.0 | 0.0 |
| C20:3ω6 (DGLA) | 0.3 | 0.3 | 0.3 |
| C20:4ω6 (ARA) | 14.1 | 23.8 | 16.3 |
| C20:3ω3 (ETrA) | 0.0 | 0.0 | 0.0 |
| C22:0 | 0.0 | 0.0 | 0.0 |
| C20:5ω3 (EPA) | 0.0 | 0.0 | 0.0 |
| C22:4ω6 (DTA) | 0.0 | 0.0 | 0.0 |
| C24:0 | 0.2 | 0.9 | 0.2 |
| Total mg | 26.4 | 2.0 | 128.0 |

### Example 6. Larger Scale Production of Polar Lipid Containing Omega-6 Fatty Acids

A series of experiments was undertaken to investigate culturing of Y. lipolytica at larger scale, specifically at 2 L, 8 L, 25 L and even larger volumes. These initial experiments used the wild-type Y. lipolytica strain W29, with provision of ω6 fatty acids by inclusion in the growth medium, for example of ARA-containing oil or free fatty acid including ARA.

### Experiment 1. Batches T-059 to T-062

In a first experiment with 2 L cultures, carried out using the bioreactor parameters as described in Example 1, ARA was provided to the Y. lipolytica cells in the form of either an unhydrolysed oil or a hydrolysed oil containing about 50% ARA. The fatty acid composition of the ARA oil is provided in Example 5, Table 12. An ARA oil hydrolysate was prepared from the ARA oil according to method 2 as described in Example 5. As the ARA oil hydrolysate was solid at room temperature, it was melted at 65° C. to enable addition to the bioreactor.

The cultures produced in this experiment were as follows.
T-059: Control culture with no added ARA oil, to establish baseline biomass yield.
T-060: The same as T-059 except 20.8 ml/L of ARA oil hydrolysate was added to the medium before inoculation.
T-061: The same as T-059 except 41.7 ml/L of ARA oil hydrolysate was added to the medium before inoculation.
T-062: The same as T-059 except 8.3 ml/L of unhydrolysed ARA oil was added to the medium before inoculation.

No specific measures were taken to mix the oil with rest of the culture since it was thought that the fermenter provided good mixing through agitation. No detergent such as NP-40 was therefore added to the medium. The cultures T-059, T-060 and T-062 were sampled at 24 and 31 h and all of the cultures harvested at 48 h. For the first 24 h of incubation, batch T-061 had a long lag phase during which there was no significant growth of the yeast, possibly due to inhibition by the ethanol used as a solvent for the addition of the hydrolysate to the medium, yielding a lower OD600 at harvest. Each pellet of harvested cells was washed with 250 ml of sterile water, reducing the pellet weight by around 5 g per pellet as a consequence of the wash.

The yield parameters for batches T-059 to 062 are listed in Table 26 and the fatty acid composition of polar lipids obtained from cells at the three time points are shown in Table 27. ARA was observed at low levels in the polar lipid in each case when ARA oil was added to the medium, at up to 2.8% of the total fatty acid content of the polar lipid, as were very low levels of DGLA which was also present in the ARA oil provided to the media. No ARA was present in the polar lipid from the control culture T-059, as expected since wild-type Y. lipolytica does not naturally produce ARA. The ω3 fatty acid content in the extracted polar lipids was very low at not more than 0.2%, and stearic acid was present at a level of not more than 3.0%. By comparing the results from T-062 with T-060 and T-061, it was concluded that addition of unhydrolysed ARA oil to the culture provided for incorporation of ARA into the polar lipid pool, better than addition of the hydrolysed ARA oil, in the context of a 2 L fermenter culture. It was also concluded that the percentage of ARA in the total fatty acid content of the polar lipid decreased from 24 h to 48 h. The biomass yields based on wet weight were quite high and acceptable.

### Experiment 2. Batches T-064 to T-069

In a second experiment with 2 L culture volumes, ARA in the form of the unhydrolysed oil was provided to the Y. lipolytica strain W29 cells at different levels in the medium, together with increased amounts of nitrogen in the form of ammonium chloride compared to the first experiment. Ammonia was also used for pH control in some cultures rather than KOH. The cultures were as follows.
T-064: As for batch T-062 (above) except that the amount of added ARA oil was increased to 20 ml/L and an extra 10 g/L ammonium chloride was added to the starting medium. 200 g/L KOH was used for pH control.
T-065: As for batch T-062 except that the amount of added ARA oil was increased to 40 ml/L and an extra 10 g/L ammonium chloride was added to the starting medium. 200 g/L KOH was used for pH control.
T-066: As for batch T-062 except that an extra 10 g/L ammonium chloride was added to the starting medium and 10% ammonia solution was used for pH control instead of 200 g/L KOH.
T-067: As for batch T-062 except that the amount of added ARA oil was increased to 20 ml/L and an extra 10 g/L ammonium chloride was added to the starting medium and 10% ammonia solution was used for pH control.
T-068: As for batch T-062 except that the amount of added ARA oil was increased to 40 ml/L and an extra 10 g/L ammonium chloride was added to the starting medium and 10% ammonia solution was used for pH control.
T-069: As for batch T-062 except that an extra 10 g/L ammonium chloride was added to the starting medium.

The cultures were sampled at 24, 27 and 30 h and the cultures harvested at 46.6 h. The yield parameters are provided in Table 26 and the fatty acid composition of polar lipid obtained from cells at the four time points are shown in Table 28. During the culturing of batch T-064, the agitator coupling of the bioreactor failed, with loss of aeration. This yielded a low OD600 as a consequence. Surprisingly, this resulted in increased and sustained levels of ARA in the polar lipid pool to 48 h for T-064 and the highest levels of ARA incorporation into the polar lipid pool in this experiment. It was also observed that increasing the amount of ARA oil in the starting medium increased incorporation of ARA into the polar lipid pool, and the percentage of ARA in the polar lipid pool decreased after 24 hours. The use of ammonia for pH control resulted in some improvement in ARA incorporation into the polar lipid pool.

### Experiment 3. Batches T-070 to T-073

In a third experiment with 2 L culture volumes, the inventors tested the effect of adding either 1 mM ethanolamine or 1 mM choline hydroxide, or both, to the medium in the context of including ARA in the form of unhydrolysed oil at 40 ml/L which was added to all cultures in this experiment. The cultures were as follows.
T-070: As for batch T-068 using ARA oil at 40 ml/L, to test the reproducibility of the system.
T-071: As for batch T-070 except that 1 mM ethanolamine was added to the starting medium.
T-072: As for batch T-070 except that 1 mM choline hydroxide was added to the starting medium.
T-073: As for batch T-070 except that 1 mM ethanolamine and 1 mM choline hydroxide were added to the starting medium.

In this experiment, the lab cooling system stopped working which resulted in the vessel temperatures increasing to 39° C., starting at 9.5 to 10 h after inoculation and remaining at about 39° C. for about 2 to 3 hours. The cultures started to cool down again about 20 to 22 h after inoculation and was around 31° C. by the harvest time at 24 h. This most likely resulted in a reduced biomass yield compared to the previous experiments. For batch T-070, there was significant foaming which resulted in loss of approximately 150 mL of culture to the over-foaming system. Likewise in batch T-072 with loss of about 350 ml culture.

The cultures were harvested at 24 h. Samples of the harvested cells were dried by freeze drying, showing that the cell pellets had a dry solids content of about 20% by weight, range 16-24%. The yield parameters are provided in Table 26 and the fatty acid composition of polar lipid extracted from cells are shown in Table 29. Polar lipids were also fractionated to separate and isolate the PC and PE fractions by TLC as described in Example 1. The fatty acid composition of these PC and PE fractions were also determined (Table 29). ARA was observed in all of the fractions, both TAG and polar lipid, up to 7.6% in polar lipid and 8.8% in the isolated PC fraction. ARA was present at a lower level, about 2.4%, in the PE relative to the PC. DGLA was present in all samples but at lower levels than ARA, consistent with the lower amount of DGLA than ARA in the ARA oil. The addition of 1 mM ethanolamine to the growth medium increased the PE and PC content of the cells substantially, yielding more extracted phospholipids. Also, the addition of ethanolamine did not appear to decrease the ARA levels in the polar lipids, showing that the addition of ethanolamine increased the yield of ARA-PE and ARA-PC. The addition of 1 mM choline also appeared to increase the yield of extractable PE and PC from the cells, although the ARA level did decrease in the polar lipids with the choline in the medium. From this experiment, it was concluded that the addition of ethanolamine was useful in increasing the yield of ARA-PE and ARA-PC. It was also concluded that increased temperature during the culturing resulted in some inhibition of growth and a reduced biomass yield. Despite the elevated temperature, there was still significant incorporation of ARA into the polar lipid pool, achieving at least 7% ARA in the total fatty acid content of the polar lipid.

### Experiment 4. Scale Up to 8 L Culture

In a fourth experiment, Y. lipolytica strain W29 was cultured in 8 L of medium in a 10 L fermenter, using the method of batch T-068. This experiment added a heat treatment step (105° C., 5 min) at the conclusion of 24 h culturing, to determine the effect of heat treatment on cell inactivation and the ARA level in polar lipids in the cells.

The yield parameters are included in Table 26. The wet cell pellet weighed 1.672 kg. The culture had a dry cell weight before heating of 70.3 g/L and 56.7 g/L after heating, indicating some loss of cell biomass through the heating, presumably to cell lysis. However, the extracted polar lipid content increased through the heating process, from 2.9% before heating to 3.8% after heating. The ARA level in the total fatty content of the polar lipid increased from 3.3% to 4.0% when the cells were heat treated. It was concluded that the heat inactivation process may have allowed more efficient extraction of polar lipids. Significantly, the ARA content in the polar lipid did not decrease through the heat treatment, but increased slightly. It was also concluded that the heat treatment could be used to inactivate yeast cells at the end of culturing without loss of ARA from the polar lipid.

The harvested cells were used in experiments to extract the polar lipids by solvent extraction using hexane/ethanol (see Example 8).

Experiment 5. Growth of Y. lipolytica in the Presence of Other ω6 Fatty Acids

Y. lipolytica strain W29 is grown at 8 L scale in the presence of DGLA in the medium, using the culture conditions as described above for batch T-068. DGLA (Nu-Chek Prep Inc., Catalog No. U-69-A) is added to the medium rather than ARA. The cells are harvested after 24 h and polar lipid extracted with hexane/ethanol. In another experiment, Y. lipolytica is grown in the presence of adrenic acid (docosatetraenoic acid, DTA, C22:4ω6) (3B Pharmachem International Co. Ltd., Wuhan, China), or docosapentaenoic acid-ω6 (DPAω6, C22:5ω6, Nu-Chek Prep, Inc.), the cells harvested and polar lipid is extracted. Combinations of the ω6 fatty acids can also be added to the medium, for example ARA together with GLA, DGLA, DTA or DPAω6, or any other combinations of two, three or even four of the ω6 fatty acids.

TABLE 26
Yeild parameters for Y. lipolytica cultures
incorporating exogenous ARA into lipids.

| | OD600 | Wet cell pellet | | Polar lipid | ARA content |
|---|---|---|---|---|---|
| Batch | at | weight | TAG content | content | in polar lipid |
| No. | harvest | (g) | (% DCW) | (% DCW) | (%) |
| T-059 | 411 | 776 | nd | 2.0 | 0 |
| T-060 | 435 | 815 | nd | 0.6 at 24 h | 1.0 at 24 h |
| T-061 | 243 | 483 | nd | 1.1 at 48 h | 0.5 at 48 h |
| T-062 | 315 | 671 | nd | 1.2 at 48 h | 2.8 at 48 h |
| T-064 | 25.4* | 29* | 2.1 | 2.3 | 11.4 at 27 h |
| T-065 | 385 | 945 | 1.5 | 1.9 at 27 h | 2.8 at 24 h |
| T-066 | 318 | 646 | 0.6 | 2.9 at 27 h | 2.6 at 24 h |
| T-067 | 330 | 723 | 0.6% at 48 h | 1.4 at 24 h | 2.9 at 27 h |
| T-068 | 312 | 706 | 2.4 at 24 h | 2.8 at 30 h | 6.6 at 24 h |
| T-069 | 360 | 816 | 2.9 at 48 h | 2.0 at 48 h | 1.7 at 24 h |
| T-070 | 109 | 148 | 2.4 at 24 h | 2.5 | 7.3 at 24 h |
| T-071 | 104 | 113 | 3.3 | 2.5 at 30 h | 7.6 at 24 h |
| T-072 | 87 | 117 | 3.0 | 2.1 at 30 h | 3.8 at 24 h |
| T-073 | 100 | 99 | 0.9 | 2.8 at 24 h | 2.8 at 24 h |
| T-074 | 261 | (70.3 g DCW/L) | 0.2 | 2.9 | 3.3 |
| before | | | | | |
| heat | | | | | |
| T-074 | | 1672 | 0.4 | 3.8 | 4.0 |
| after heat | | (56.7 g DCW/L) | | | |

TABLE 27
Fatty acid composition of polar lipid from Y. lipolytica cells cultured
in the presence of an ARA oil (hydrolysate or unhydrolyzed).

| | | | | | | | | C18:2ω6 | C18:3ω6 |
|---|---|---|---|---|---|---|---|---|---|
| | C14:0 | C16:0 | C16:1Δ7 | C16:1Δ9 | C18:0 | C18:1Δ9 | C18:1Δ11 | (LA) | (GLA) |
| No ARA oil added | | | | | | | | | |
| T-059 24 h | 0.1 | 13.1 | 0.8 | 7.1 | 1.1 | 20.3 | 0.7 | 54.9 | 0.0 |
| T-059 31 h | 0.1 | 12.2 | 0.7 | 9.7 | 1.0 | 40.3 | 0.8 | 34.6 | 0.0 |
| T-059 48 h | 0.1 | 13.3 | 2.0 | 7.3 | 1.9 | 46.9 | 0.6 | 27.5 | 0.0 |
| 25 ml/L hydrolysate added | | | | | | | | | |
| T-060 24 h | 0.1 | 13.3 | 2.2 | 5.5 | 1.6 | 25.7 | 0.5 | 48.6 | 0.4 |
| T-060 31 h | 0.1 | 10.6 | 1.7 | 7.9 | 1.5 | 42.2 | 0.6 | 34.0 | 0.2 |
| T-060 48 h | 0.2 | 15.1 | 2.2 | 7.1 | 3.0 | 46.5 | 0.5 | 24.7 | 0.1 |
| 50 ml/L hydrolysate added | | | | | | | | | |
| T-061 48 h | 0.1 | 17.3 | 1.7 | 6.3 | 1.4 | 38.8 | 0.6 | 31.4 | 0.2 |
| 10 ml/L unhydrolyzed oil added | | | | | | | | | |
| T-062 24 h | 0.1 | 11.5 | 1.3 | 3.4 | 1.4 | 38.7 | 0.6 | 37.4 | 1.3 |
| T-062 31 h | 0.1 | 14.9 | 0.9 | 5.1 | 2.2 | 40.0 | 0.4 | 33.9 | 0.5 |
| T-062 48 h | 0.1 | 14.3 | 1.6 | 6.7 | 1.9 | 47.2 | 0.4 | 26.6 | 0.2 |

| | C18:3ω3 | | | | C20:3ω6 | C20:4ω6 | | | Wt %/ |
|---|---|---|---|---|---|---|---|---|---|
| | (ALA) | C20:0 | C20:1Δ11 | C20:2ω6 | (DGLA) | (ARA) | C22:0 | C24:0 | DCW |
| No ARA oil added | | | | | | | | | |
| T-059 24 h | 0.1 | 0.0 | 0.6 | 1.2 | 0.0 | 0.0 | 0.0 | 0.1 | 1.5 |
| T-059 31 h | 0.0 | 0.0 | 0.3 | 0.2 | 0.0 | 0.0 | 0.0 | 0.1 | 2.0 |
| T-059 48 h | 0.0 | 0.0 | 0.2 | 0.0 | 0.0 | 0.0 | 0.0 | 0.2 | 0.7 |
| 25 ml/L hydrolysate added | | | | | | | | | |
| T-060 24 h | 0.1 | 0.0 | 0.4 | 0.4 | 0.2 | 1.0 | 0.0 | 0.0 | 0.4 |
| T-060 31 h | 0.1 | 0.0 | 0.2 | 0.2 | 0.1 | 0.6 | 0.0 | 0.0 | 0.6 |
| T-060 48 h | 0.0 | 0.0 | 0.1 | 0.1 | 0.0 | 0.2 | 0.0 | 0.2 | 0.5 |
| 50 ml/L hydrolysate added | | | | | | | | | |
| T-061 48 h | 0.1 | 0 | 0.2 | 0.1 | 0.1 | 0.5 | 0 | 1.2 | 1.1 |
| 10 ml/L unhydrolyzed oil added | | | | | | | | | |
| T-062 24 h | 0.2 | 0.0 | 0.4 | 0.4 | 0.4 | 2.8 | 0.0 | 0.1 | 0.5 |
| T-062 31 h | 0.1 | 0.0 | 0.3 | 0.1 | 0.2 | 1.1 | 0.0 | 0.2 | 0.4 |
| T-062 48 h | 0.0 | 0.0 | 0.1 | 0.1 | 0.1 | 0.5 | 0.0 | 0.2 | 1.1 |

TABLE 28
Fatty acid composition of polar lipid from Y. lipolytica
cells cultured in the presence of an ARA oil (unhydrolyzed).

| | | | | | | | C18:2ω6 | C18:3ω6 | C20:4ω6 |
|---|---|---|---|---|---|---|---|---|---|
| | C14:0 | C16:0 | C16:1Δ9 | C18:0 | C18:1Δ9 | C18:1Δ11 | (LA) | (GLA) | (ARA) |
| 20 ml/L oil added | | | | | | | | | |
| T-064 24 h | 0.3 | 22.0 | 2.0 | 1.6 | 23.6 | 1.0 | 31.2 | 7.1 | 11.2 |
| T-064 27 h | 0.3 | 22.0 | 2.1 | 1.6 | 23.1 | 1.0 | 31.5 | 7.0 | 11.4 |
| T-064 30 h | 0.0 | 21.9 | 2.3 | 1.8 | 22.9 | 0.0 | 33.4 | 6.6 | 11.1 |
| T-064 46.6 h | 0.2 | 21.1 | 1.8 | 1.2 | 26.1 | 1.0 | 30.9 | 7.4 | 10.3 |
| 40 ml/L oil added | | | | | | | | | |
| T-065 24 h | 0.0 | 19.9 | 1.4 | 2.1 | 33.5 | 0.7 | 37.9 | 1.8 | 2.8 |
| T-065 27 h | 0.0 | 14.8 | 4.3 | 0.7 | 38.4 | 0.5 | 38.9 | 1.1 | 1.3 |
| T-065 30 h | 0.0 | 14.2 | 6.0 | 0.7 | 40.0 | 0.5 | 36.8 | 0.9 | 0.9 |
| T-065 46.6 h | 0.0 | 10.7 | 9.9 | 0.0 | 49.3 | 0.7 | 29.5 | 0.0 | 0.0 |
| 10 ml/L oil added | | | | | | | | | |
| T-066 24 h | 0.2 | 12.0 | 6.3 | 0.5 | 37.7 | 0.7 | 39.0 | 0.9 | 2.6 |
| T-066 27 h | 0.2 | 10.9 | 7.3 | 0.4 | 42.6 | 0.7 | 36.8 | 0.5 | 0.7 |
| T-066 30 h | 0.2 | 10.9 | 8.1 | 0.3 | 44.3 | 0.7 | 34.6 | 0.4 | 0.4 |
| T-066 46.6 h | 0.0 | 10.2 | 9.3 | 0.3 | 47.9 | 0.7 | 31.5 | 0.0 | 0.0 |
| 20 ml/L oil added | | | | | | | | | |
| T-067 24 h | 0.2 | 12.8 | 5.0 | 0.6 | 38.8 | 0.7 | 39.5 | 1.0 | 1.4 |
| T-067 27 h | 0.2 | 11.6 | 5.9 | 0.5 | 43.7 | 0.7 | 36.0 | 0.8 | 0.8 |
| T-067 30 h | 0.2 | 11.3 | 6.8 | 0.4 | 45.5 | 0.6 | 34.0 | 0.6 | 0.6 |
| T-067 46.6 h | 0.0 | 10.7 | 8.4 | 0.3 | 48.8 | 0.6 | 31.1 | 0.0 | 0.0 |
| 40 ml/L oil added | | | | | | | | | |
| T-068 24 h | 0.0 | 19.0 | 1.1 | 1.1 | 26.3 | 1.1 | 41.4 | 3.5 | 6.6 |
| T-068 27 h | 0.0 | 18.8 | 1.5 | 1.6 | 21.8 | 1.0 | 48.0 | 2.7 | 4.6 |
| T-068 30 h | 0.0 | 17.1 | 3.5 | 0.8 | 26.5 | 0.7 | 48.2 | 1.4 | 1.8 |
| T-068 46.6 h | 0.0 | 11.7 | 5.1 | 0.5 | 47.5 | 0.6 | 33.4 | 0.6 | 0.6 |
| 10 ml/L oil added | | | | | | | | | |
| T-069 24 h | 0.0 | 12.5 | 6.5 | 0.5 | 37.6 | 0.5 | 40.1 | 0.7 | 1.7 |
| T-069 27 h | 0.0 | 11.9 | 8.2 | 0.4 | 40.4 | 0.4 | 38.0 | 0.0 | 0.6 |
| T-069 30 h | 0.0 | 12.6 | 9.5 | 0.5 | 40.9 | 0.0 | 36.4 | 0.0 | 0.0 |
| T-069 46.6 h | 0.0 | 10.3 | 12.5 | 0.5 | 43.6 | 0.7 | 32.5 | 0.0 | 0.0 |

TABLE 29
Fatty acid composition of TAG, total polar lipid and PC and PE classes of PL from
Y. lipolytica cells cultured in the presence of an ARA oil, with or without
added 1 mM ethanolamine or 1 mM choline to the medium, 2 L scale (Experiment 3).

| | | | | | | | C18:2ω6 | C18:3ω3 | C18:3ω6 | |
|---|---|---|---|---|---|---|---|---|---|---|
| | C14:0 | C16:0 | C16:1Δ9 | C18:0 | C18:1Δ9 | C18:1Δ11 | LA | ALA | GLA | C20:0 |
| TAG | | | | | | | | | | |
| T-070 24 h | 0.0 | 12.0 | 1.0 | 7.0 | 27.9 | 1.0 | 26.9 | 0.4 | 5.1 | 0.2 |
| T-071 24 h | 0.1 | 13.6 | 1.0 | 6.0 | 25.5 | 1.0 | 28.0 | 0.5 | 5.3 | 0.3 |
| T-072 24 h | 0.0 | 13.9 | 1.0 | 7.9 | 28.7 | 1.1 | 28.5 | 0.5 | 3.4 | 0.3 |
| T-073 24 h | 0.0 | 12.0 | 1.2 | 8.2 | 30.5 | 1.2 | 29.5 | 0.4 | 2.9 | 0.4 |
| Polar | | | | | | | | | | |
| lipid | | | | | | | | | | |
| T-070 24 h | 0.0 | 18.0 | 0.9 | 1.9 | 30.1 | 0.7 | 34.7 | 0.2 | 2.8 | 0.1 |
| T-071 24 h | 0.1 | 17.1 | 0.9 | 1.3 | 30.4 | 0.7 | 35.8 | 0.3 | 3.1 | 0.0 |
| T-072 24 h | 0.1 | 17.2 | 1.3 | 2.4 | 24.2 | 0.8 | 44.1 | 0.3 | 2.1 | 0.2 |
| T-073 24 h | 0.1 | 15.8 | 2.0 | 2.2 | 25.6 | 0.8 | 45.2 | 0.3 | 1.7 | 0.2 |
| PC | | | | | | | | | | |
| T-070 24 h | 0.0 | 14.7 | 1.2 | 1.6 | 29.2 | 1.2 | 36.3 | 0.0 | 4.3 | 0.0 |
| T-071 24 h | 0.0 | 14.4 | 1.2 | 1.1 | 29.9 | 1.1 | 35.6 | 0.4 | 4.6 | 0.0 |
| T-072 24 h | 0.0 | 13.8 | 1.8 | 1.4 | 21.0 | 1.5 | 49.4 | 0.4 | 3.1 | 0.0 |
| T-073 24 h | 0.0 | 12.0 | 2.7 | 1.5 | 23.3 | 1.6 | 51.3 | 0.4 | 2.4 | 0.0 |
| PE | | | | | | | | | | |
| T-070 24 h | 0.0 | 30.0 | 0.9 | 1.2 | 35.7 | 0.5 | 27.7 | 0.0 | 1.7 | 0.0 |
| T-071 24 h | 0.0 | 30.3 | 0.8 | 0.6 | 35.4 | 0.5 | 27.6 | 0.0 | 1.8 | 0.0 |
| T-072 24 h | 0.0 | 25.7 | 1.3 | 1.3 | 28.6 | 0.6 | 37.6 | 0.0 | 1.4 | 0.0 |
| T-073 24 h | 0.0 | 24.9 | 2.4 | 1.1 | 31.1 | 0.6 | 37.7 | 0.0 | 1.0 | 0.0 |

| | | | | | C20:3ω6 | C20:4ω6 | | | Wt %/ |
|---|---|---|---|---|---|---|---|---|---|
| | C20:1Δ11 | C20:1Δ5 | C20:2ω6 | C20:3ω3 | DGLA | ARA | C22:0 | C24:0 | DCW |
| TAG | | | | | | | | | |
| T-070 24 h | 0.2 | 0.0 | 0.7 | 0.3 | 1.9 | 14.2 | 0.3 | 0.8 | 2.4 |
| T-071 24 h | 0.2 | 0.0 | 0.5 | 0.2 | 1.6 | 15.0 | 0.3 | 0.7 | 3.3 |
| T-072 24 h | 0.3 | 0.0 | 0.6 | 0.3 | 1.5 | 10.3 | 0.5 | 1.1 | 3.0 |
| T-073 24 h | 0.3 | 0.0 | 0.6 | 0.5 | 1.6 | 8.9 | 0.6 | 1.3 | 1.9 |
| Polar | | | | | | | | | |
| lipid | | | | | | | | | |
| T-070 24 h | 0.3 | 0.6 | 0.8 | 0.3 | 1.2 | 7.3 | 0.1 | 0.1 | 1.9 |
| T-071 24 h | 0.3 | 0.6 | 0.6 | 0.3 | 1.0 | 7.6 | 0.1 | 0.1 | 2.5 |
| T-072 24 h | 0.4 | 0.5 | 0.8 | 0.2 | 1.1 | 3.8 | 0.2 | 0.2 | 2.1 |
| T-073 24 h | 0.3 | 0.5 | 0.7 | 0.1 | 0.9 | 2.8 | 0.2 | 0.2 | 2.8 |
| PC | | | | | | | | | |
| T-070 24 h | 0.0 | 0.0 | 1.1 | 0.0 | 1.7 | 8.8 | 0.0 | 0.0 | 0.55 |
| T-071 24 h | 0.4 | 0.0 | 0.9 | 0.5 | 1.3 | 8.5 | 0.0 | 0.0 | 0.82 |
| T-072 24 h | 0.5 | 0.0 | 1.2 | 0.0 | 1.5 | 4.3 | 0.0 | 0.0 | 0.68 |
| T-073 24 h | 0.5 | 0.0 | 1.1 | 0.0 | 1.1 | 2.2 | 0.0 | 0.0 | 0.74 |
| PE | | | | | | | | | |
| T-070 24 h | 0.0 | 0.0 | 0.0 | 0.0 | 0.1 | 2.2 | 0.0 | 0.0 | 0.32 |
| T-071 24 h | 0.0 | 0.0 | 0.0 | 0.0 | 0.5 | 2.4 | 0.0 | 0.0 | 0.41 |
| T-072 24 h | 0.0 | 0.0 | 0.5 | 0.0 | 0.6 | 2.3 | 0.0 | 0.0 | 0.49 |
| T-073 24 h | 0.0 | 0.0 | 0.4 | 0.0 | 0.1 | 0.8 | 0.0 | 0.0 | 0.49 |

### Experiment 6 (B001)

In a first experiment at a 25 L scale, wild-type Y. lipolytica strain W29 was grown in a 25 L fermenter to test biomass production, recovery and drying, and lipid extraction processes from a batch culture at the larger scale. Lipid accumulation was also monitored at different time points in this culture. The growth medium was as described in Table 1 of the review by Hahn-Hägerdal et al. (2005), DM column, page 5 with the following adjustments: all vitamins were omitted except thiamine. Potassium dihydrogen phosphate was added at 10 g/L, ammonium sulphate was replaced with diammonium phosphate at 10 g/L, citric acid was added at 2 g/L. The trace elements CuSO₄, NaMoO₄, MnCl₂, CoCl₂, H₃BO₃ and ZnSO₄ were present in reduced concentrations to the published recipe by varying amounts of between 3 and 30-fold and CaCl₂) was increased by a factor of 8. Additional S, N and P were supplied as inorganic acids. The culture medium was sterilised in the fermenter by autoclaving and thiamine added aseptically after the heat treatment to a final concentration of 0.15 g/L using a 200 g/L sterile filtered thiamine stock solution. The growth medium had 40 g/kg glycerol as the carbon source, pH 6.0 at the beginning of culture. One advantage of this medium was that it could be sterilised by autoclave as a whole, with addition after autoclaving of only the thiamine.

The strain W29 inoculum for the fermenter culture was grown as a 400 ml culture in YPD medium, in flasks at 29° C. with shaking at 180 rpm for 24 h. The inoculum was added to the fermenter and the mixture sampled to provide a time zero sample. After inoculation, the OD600 of the culture was 0.132. The culture conditions were: temperature at 29° C., the pH set point was 6.0, the airflow was approximately 33 L/min and the stirrer approximately 200 RPM. The following parameters were monitored-dissolved oxygen (DO) concentration and pH. The temperature and pH values were controlled to the respective set-points. The culture pH value was changed from 6.0 to 8.0 at the 47 h time-point (post inoculation) to stimulate the accumulation of lipid. Growth of the yeast was monitored by measuring OD at 600 nm (OD600). The level of citric acid in the medium was also monitored since wild-type Y. lipolytica secretes citrate during growth on glycerol as carbon source. The DO decreased during culture from 10 ppm to about 2 ppm at 48 h. A metabolite with the HPLC retention time consistent with citric acid was produced in the culture and accumulated gradually, reaching about 40 g/L at 60 h but then declining to about 33 g/L at 90 h. The concentration of glycerol decreased gradually to about zero at 40 h, at which time a glycerol feed was supplied to the culture using 4.5 L of 400 g/kg glycerol over the next 8 h. This increased the glycerol concentration in the medium to about 20 g/kg, after which the glycerol concentration decreased to zero at 60 h timepoint. At 90 h timepoint, the cell density had reached about 30 g/kg (DCW), at which time the culturing was ceased and the cells harvested by centrifugation. The biomass was washed with 2 volumes of cold water, providing a yeast cream of 3.2 kg having 17% solids. Half of the biomass was spray dried with an inlet temperature of 160° C., outlet temperature of 78° C., yielding 156 g of dried powder. The remaining 1.7 kg having 20% solids was frozen. A small portion of this material was freeze dried, recovering 22 g of dried cells.

Samples were removed at 42, 62 and 68 h during the culturing and at 90 h at harvest of the cells. The samples were either spray dried or freeze dried and analysed for lipid content by extraction of lipid using ethanol/hexane (60/40; v/v) as solvent for 20 h. The solvent of the extracted lipid was evaporated under vacuum at 50° C., and the lipid dried under a stream of CO₂. The dried lipid was weighed. At each of the timepoints from 40 h to 90 h, the lipid concentration was between 17-25% on a dry cell weight basis. To analyse the composition, samples of the extracted lipid were dissolved in 2 ml of ethanol/hexane (6/4 v/v) or 1 ml chloroform and 2×5 μl aliquots chromatographed on a TLC plate (Silica gel 60 F254, 25 cm×25 cm) using hexane/diethylether/acetic acid (70/30/1; v/v/v). The plates were stained with iodine vapor for 30 min to observe the lipid types. Bands were observed for polar lipids at the origin of the TLC plate, and, with increasing mobility, for DAG, free fatty acids (FFA) and TAG, with the TAG bands by far the most intense.

In this experiment, 2.8 kg of glycerol was fed to the culture after the initial batch phase had ended as determined by the exhaustion of glycerol at around 40 hours post inoculation. Almost equal quantities of citric acid and biomass were produced by the end of the experiment at 90 h post inoculation. Interestingly, the highest level of extracted lipids per gram of cell dry matter occurred at the point when glycerol from the nutrient feed was exhausted at 61.5 h post inoculation. Further lipid generation was not observed between this point and when the experiment ended at 90 h. This fermentation produced a lower cell density than experienced at a 2 L scale but significantly higher TAG accumulation at about 30% vs 5% on a dry cell weight basis. This experiment out to 90 h was designed for large amounts of TAG production after nitrogen limitation was achieved. Improvements were considered where cell density was increased whilst maintaining good TAG production, most likely through balancing the nitrogen and glycerol concentrations. The concentration of citric acid declined late in the fermentation suggesting this can be used as a carbon source when glycerol is exhausted.

It was demonstrated that the yeast cells could be successfully harvested by centrifugation and that culture broth could be removed from the cells by resuspending the cell pellet in cold water and then re-centrifuging the cells to a paste. It was also demonstrated that the biomass could be freeze dried from frozen paste or spray dried from a yeast cream of 20% solids. It was concluded from the TLC analysis that at least 30% of the solvent extract from the cells was TAG, the remaining lipid mass made up by DAG>FFA and polar lipid, in that order of abundance.

### Experiment 7 (B002)

In a second experiment at the 25 L scale, wild-type Y. lipolytica strain W29 was grown in a fermenter with the addition of an ARA feed to assess incorporation of the fatty acid into both TAG and polar lipids, as well as biomass production in the presence of the fed fatty acid in the context of a batch culture at the larger scale. As the experiment aimed to incorporate ARA into the phospholipids and to provide for a greater ratio of PL:TAG, the culturing was terminated earlier at 17 h rather than at 90 h. The growth medium was also different than in the first 25 L experiment, based on a richer YPD medium which favoured biomass production rather than TAG production. The base medium contained Yeast Extract at 3 g/L, Malt Extract at 3 g/L, Soy peptone at 5 g/L and dextrose monohydrate as the main carbon source at 10 g/L. The pH was initially adjusted to 6.0, although the pH at the start of culture was 7.31. This medium was prepared and sterilised in the fermenter by autoclaving. When the medium had cooled, the ARA was added aseptically to the medium in the form of an emulsion made from 100 ml hydrolysed ARA oil (see Example 5), 8 ml of a solution containing an 8 g sample of 90% pure ARA (NuChek, Catalog No. U-71-A) and 250 ml of non-hydrolysed ARA oil (Jinan Boss Chemical Industry Co. Ltd, China), emulsified with 50 ml Triton X-100 mixed with 50 ml water. The ARA emulsion was added to the fermenter via a dosing pump. This provided a final concentration of the surfactant at 0.2% (w/v) Triton X-100 to emulsify the oil. A lipase solution containing 3.5 g of fungal lipase (Fungal lipase 8000, Connell Brothers) in 50 ml water was then injected via a sterile port, providing an added lipase initial concentration of 0.13 g/L. This was intended to provide for gradual hydrolysis of the ARA oil. Finally, when the temperature was 29° C., 400 ml of W29 inoculum was added to the fermenter by overpressure, providing an initial cell density (OD600) of 0.11. The inoculum had been prepared by growing a 400 ml culture of W29 in YPD medium for 24 h at 29° C., to an OD600 of 5.33.

Initial fermentation parameters at inoculation were DO-8.6, pH=7.31, air=15 ml/min, agitation was at 5% of full speed and the back pressure was 15 psi. Agitation and air flow were low to avoid excessive foaming from the surfactant. Backpressure was applied to ensure good oxygen transfer. The presence of oil and surfactant prevented the use of OD600 and HPLC to monitor the sugar concentration and OD development since the oil and surfactant absorbed strongly at OD600. As a result, acid production was used to monitor the culture growth. The initial pH was 7.31, decreasing to about 4.0 over the course of the culturing. The pH was not controlled in this experiment, following a previous shake-flask protocol. According to acid production, exponential growth started 6-7 hours after inoculation and began to slow 12-13 hours after inoculation. The cellular growth may have slowed due to carbon limitation or because it reached a sub-optimal pH. The start medium contained 10 g/L glucose and 3 g/L maltose and if all was consumed at maximal yield, the yeast cell density was expected to be about 6.5 g/L assuming a 50% conversion of sugars to biomass. The observed yield at harvest was 7.1 g/L, so it was considered possible that some of the oil was also used as a carbon source. This cell density and the fermentation time-frame suggested that the culture was harvested before stationary phase was reached, i.e. corresponded to a late log-phase of growth. The culturing was terminated at 17 h by heating the culture to 76-80° C. for 5 min, after which the mixture was cooled to 10° C. The biomass concentration was 7.06 g/L, composed mostly of single cells with some elongated but short pseudo-hyphae as observed by light microscopy.

The cells were collected from 26 L of broth by centrifugation at 4,750 rpm for 5 min at 5° C., providing three fractions: a semi-solid fat emulsion, an aqueous supernatant and a cell paste. The cell paste was washed by resuspending the cell pellet in 3-times the pellet volume of cold sodium chloride solution (0.9 g/L) and the cells collected by centrifugation. This yielded 541 g of cell paste which contained 28% solids. A 46 g portion of the paste was freeze dried for total lipid and TLC analysis, providing 12 g of dried yeast powder. The remaining 495 g pf paste was freeze dried in 120 g portions and milled to a powder. The yield of yeast was 150 g on a dry cell basis, washed and dried, which corresponded to a recovery 5.77 g yeast per litre of culture broth. An ethanol/hexane extraction of a sample of the cells provided 26% by dry cell weight of extractable lipid. The spent culture medium was also analysed.

This fermentation aimed to incorporate ARA into the lipid membranes of rapidly growing Y. lipolytica. The culture grew very well in the presence of surfactant, a combination of ARA as free fatty acid and ARA oil in the form of TAG, and the lipase, growing from an OD600 of 0.11 to an OD600 of about 7, corresponding to a cell density of 7 g/L. The cells had a significant oil content of 26% of dry cell weight. The TLC analysis showed the presence of TAG, DAG and MAG in the freeze-dried yeast. Polar lipids and free fatty acids were also observed.

### Experiments 8 and 9 (B003, B004))

In further experiments at the 25 L scale, wild-type Y. lipolytica strain W29 was grown in a medium using 40 g/L or 70 g/L glycerol as carbon source in order to assess biomass production and lipid production at various C/N ratios. In experiment B003, the initial medium had 40 g/L glycerol, thiamine at 0.15 g/L, pH 6.0 and no added citric acid, relative to experiment B001. As nitrogen source, di-ammonium phosphate (DAP) was present at 10 g/L to encourage biomass growth during the batch stage. The initial C:N ratio was therefore initially 6:1. The initial cell density (OD600) after addition of the 1 L inoculum of W29 cells was 0.22 and the pH 6.22. The biomass reached a DCW of 15.7 g/L at 17 h. At 20-hours post inoculation, the feed (5 L) including glycerol and DAP was started at 0.5 L per hour, providing a C:N ratio of 20:1. The biomass continued to grow in exponential phase to 30.51 g/L at 24 h, equal to half of the feed, indicating there was remaining nitrogen in the batch medium at 20 h. After that the biomass reached stationary phase suggesting that a critical nutrient was limited. As there was excess glycerol and citric acid in the 24 h culture sample, the limiting nutrient was likely to be nitrogen. From the 24 h time-point, the citric acid concentration increased to a maximum of 14 g/L while glycerol was steadily consumed to a minimum of 0.46 g/L by the 30 h time-point when the feed medium was consumed. At the end of the fermentation experiment at 45 h, the DCW was 34.3 g/L, and the citric acid concentration was 7.69 g/L. In contrast to experiment B001, the harvest biomass DCW was increased from 29.6 to 34.3 g/L and the citric acid concentration in the harvest supernatant reduced from 33.45 to 7.69 g/L. However, the ethanol/hexane extractable lipid content was reduced from 28% in B001 to 8% in B003. The recovered cells were washed with 2 volumes of cold 1% NaCl (w/v) and spray dried. In conclusion, 671 g of washed yeast powder was produced from 30 L of fermenter broth. Nutrients were more efficiently converted to biomass than in experiment B001 but less lipid accumulated. Prolonged fermentation was expected to increase the lipid yield.

Experiment B004 modified the initial medium to 70 g/L glycerol, to provide for earlier nitrogen limitation between 24-30 h before the glycerol feed began. The feed included a mineral salt mixture with some nitrogen, as the sole nitrogen source. The DAP was omitted from the feed in order to increase the C:N ratio from 20:1 to approximately 100:1. A change in oxygen consumption or glycerol depletion was monitored to signal the start of the feed. A biomass of 50.1 g/L was achieved at the 32 h timepoint. At the end of Feed 1, the residual glycerol and citric acid were 18 g/L and 15 g/L, respectively. Batch fermentation after Feed 1 continued to 47 h. 7 L broth was harvested, concentrated, washed and frozen. It was noted that 50% of the cells stained with methylene blue after the extended cultivation at pH 6 after Feed 1. The dissolved oxygen level rose dramatically indicating that the culture was metabolically inactive, most likely due to nitrogen exhaustion.

As there was a significant population of live cells, a second feed started with the same composition as Feed 1, but the pH was adjusted to 8 to investigate if this might result in lipid storage in the remaining viable cells. After the second feed, the biomass increased slightly from 43 to 47 g/L. The residual glycerol and citric acid continually increased to 23 g/L and 25 g/L, respectively, during the second feed. The batch fermentation continued for another 16 h after the end of Feed 2. By the end of the experiment, citric acid had increased to 40 g/L and the residual glycerol had reduced to 9 g/L. The fermentation was stopped at 77 h. 7 L of the culture was harvested before heat treatment to evaluate the impact of heat treatment on cell composition. Before heat treatment, the biomass dry weight was 41 g/L, neutral lipid content was 18% w/w and the glycerol and citric acid were 9 g/L and 40 g/L respectively. The remaining culture was treated by heating to 105° C. for 5 min. The DCW was 41 g/L, and lipid content was 0.8% w/w, which indicated the lipid was released to the supernatant as a result of the heat inactivation step.

It was concluded from these experiments that large scale fermentation could produce yeast cells with suitable biomass and lipid production.

### Experiment 10 (B005)

In a larger scale experiment with 25 L of culture, wild-type Y. lipolytica strain W29 was grown in a Braun fermenter with the addition of ARA to the medium, seeking to produce more cell biomass, increase the polar lipid: TAG ratio and improve the incorporation of ω6 fatty acid into polar lipids. As the experiment aimed to incorporate ω6 fatty acid into the phospholipids and to provide for a greater ratio of PL:TAG, the fermentation was terminated towards the end of active growth rather than in stationary phase, as follows. The growth medium was based on a rich YPD medium which favoured biomass production rather than TAG production. The base medium contained Yeast Extract at 3 g/L, Malt Extract at 3 g/L, Soy peptone at 5 g/L and dextrose monohydrate as the main carbon source at 10 g/L. The pH was initially adjusted to 6.0. This medium was prepared and sterilised in the fermenter by autoclaving in situ, then cooled by direct cooling to the fermenter jacket. After the medium had cooled to 29° C., ARA was added aseptically by overpressure to the medium in the form of 12.5 g ARA (NuChek) as free fatty acid in 300 ml of 17% Triton-X-100 to give a final concentration in the fermenter of 0.5 g/L ARA and 0.2% Triton-X-100, with further addition of 100 ml of unhydrolyzed ARA oil to provide a concentration of 0.4% (v/v) unhydrolyzed ARA oil in addition to the FFA. A seed culture was prepared in 400 ml YM medium at 29° C. with shaking at 180 rpm overnight, providing an inoculum having an OD600 of 4.23. When the medium temperature was 29° C., 400 mL of the seed culture was transferred to the fermenter by overpressure, providing an initial cell density (OD600) of 0.07 by calculation.

The initial fermentation parameters at inoculation were DO at 7.92, pH 7.01, air introduction at 10 ml/min, agitation at 5% of full speed, and back pressure at 11 psi. The initial OD600 was 3.35, almost entirely from the surfactant/oil emulsion, so DO, citric acid production and pH changes were tracked to follow logarithmic growth. In particular, these parameters were followed after about 15 h post inoculation for signs that log-growth was slowing. Agitation and air flow were low to avoid excessive foaming from the surfactant. The backpressure (11 psi) was applied to ensure good oxygen transfer at the low agitation speed. The pH was not controlled. Almost no antifoam (20% Silfax D3 food grade) was used during this experiment.

According to citric acid production, exponential growth started 6-7 h after inoculation and began to slow 16 h after inoculation when the broth was chilled and the cells harvested by centrifugation. The growth may have slowed due to carbon limitation or because it reached a sub-optimal pH. The start medium contained 10 g/L glucose and 3 g/L maltose and if all was consumed at maximal yield, the yeast cell density was expected to be about 6.5 g/L assuming 50% yield. The DCW at harvest was 4.2 g/L. This demonstrated that carbon limitation had likely not been reached which was consistent with the objective to harvest the cells at late log-phase to avoid carbon limitation and subsequent digestion of ARA-PL. The culture was terminated at late logarithmic growth phase to maximise polar lipid content and ARA incorporation and was not heat treated at the end of the fermentation. At harvest, the cells were budding as observed by light microscopy and there were very few that stained with Methylene Blue, so the oil content and therefore the TAG content was low as intended. A final yield of 294 g of wet paste was obtained from the 21 L of culture, with approximately 72% water content i.e. approximately 28% w/w solids. The cell paste was frozen and then freeze dried in 3 batches to yield 73 g of dry yeast cake. The dry yeast cake was milled to a fine powder and dispensed as 3 portions-a 3 g portion for lipid analysis, a 35 g portion for food application trials and a 35 g portion for further processing to yield a crude lipid fraction.

Lipid was extracted from 35 g of yeast powder by adding 900 mL of 60% hexane/40% dry ethanol in a 1 L bottle. The bottle was shaken in an orbital shaker at 180 rpm for 4 h at 29° C. The yeast powder was well suspended in the solvent using this approach. After 4 h of extraction, the solvent was filtered into a glass flask using a ceramic Buchner funnel and a glass filter (Advantec GA-100, 125 mm diameter). Some yeast debris bypassed the filter so the solution was re-filtered by gravity into a 2 L round bottom flask. The solvent was evaporated under vacuum to a final volume of approximately 20 mL and transferred to a glass culture tube for shipment.

As shown in Table 31, the fatty acid composition of the polar lipid fraction from the extracted lipid included 16.4% ARA, as well as 25% of LA. There were also smaller amounts of the other ω6 fatty acids GLA, EDA and DGLA present in the total fatty acid content, and a trace amount of the ω3 fatty acid ALA. Monounsaturated fatty acids were present included 32.7% oleic acid, the most prevalent fatty acid in the polar lipid fraction, and 7.4% palmitoleic acid. Saturated fatty acids (SFA) were present at lower amounts, predominantly palmitic acid at 12.7% and surprisingly low levels of stearate at 0.5% in the total fatty acid content of the polar lipid fraction. In contrast, the fatty acid composition of the TAG fraction was different, including 22.1% ARA. Other ω6 fatty acids were either absent or lower than in the polar lipids, for example LA at 16.7%. Again, oleic acid was the predominant fatty acid in the TAG fraction. In this experiment, where the inventors intended to not produce much TAG through the culture conditions used, the TAG content was indeed low, with a favourable polar lipid: TAG ratio of about 20 in the total lipid content.

### Further Larger Scale Production of Phospholipids Having ω6 Fatty Acid (B009)

Several experiments were carried out in a similar manner to B005 at the 25 L scale except with some modifications to the culture medium and conditions in an attempt to increase the biomass yield per litre while maintaining the level of incorporation of ARA into PL after supplementation. In experiment B009, three different fungal lipases (100 mg each) were added to the culture medium with the aim of assisting with hydrolysis of the ARA oil and incorporation of the ARA, even though Y. lipolytica is known to produce and excrete TAG lipases. Additionally, the ARA as FFA and the ARA unhydrolysed oil were first mixed with the 200 mL inoculum and then delivered to the fermenter. The non-ionic surfactant Triton X-100 was therefore added to the YPD broth before sterilisation, at the same final concentration as previously used (0.2% v/v), and autoclaved in situ with the broth.

The dissolved oxygen (DO) probe provided unexpectedly low readings 20 min post inoculation, hence the pH, OD and dry weight were the only parameters used to monitor growth of the culture in this experiment. The pH of the culture medium was not controlled in this experiment, falling from pH 6.7 to 3.3 at 16 h due acid production from cellular metabolism. The cell density (dry weight) was 9.4 g/L at 16 h, while optical density of washed cell samples increased from 0.1 to 29.3 at time 0 and 16 h, respectively. There was no bacterial growth observed during the fermentation process as determined by tests for coliforms and Salmonella, and aerobic plate count. The culture was chilled at 16 h post inoculation, the cells harvested and the cell pellets washed three times with cold deionised water. The cell paste was then heat treated at a temperature above 76° C. and below 82° C. for 3 min, aiming to inactivate the cells, then chilled by immersing the container in a water bath with ice. The fermentation terminated at 16 h produced a wet cell paste of 1390 g having a dry cell weight of 236 g. The cell paste was freeze dried.

Lipid was extracted from biomass samples using 25 mL 60% hexane: 40% ethanol as solvent per gram of the freeze-dried cells, for 3.5 h at 30° C. The solvent extracts were evaporated under vacuum at 50° C. and then dried under CO₂ gas at 10 L/min. The total lipid content of the 16 h freeze-dried sample was 4.6% on a dry weight basis. The extracted lipid was resuspended in chloroform at a concentration of 200 mg/mL and chromatographed on a TLC plate as before. The TLC results showed substantial amounts of polar lipid had been extracted from the 16 h cells. The ARA levels in the lipid extracted from the biomass when analysed by GC were 7.7% and 2.6% in TAG and PL, respectively, and 2.4% and 2.5% of the total fatty acid content in the TAG and polar lipid fractions, respectively. In this B009 experiment, the biomass production was much greater, but the ARA incorporation rate was reduced. There therefore appeared to be an inverse relationship between the amount of biomass produced and the level of ARA incorporation.

### Experiments B012 and B013

The previous experiments at 25 L scale with Y. lipolytica strain W29 were all cultured in YPD broth with the addition of 0.2 mL/L Triton X-100 to solubilise 100 mL of ARA oil and 10 g ARA as FFA. All of the ferments were terminated at about 16 h. Those experiments varied in terms of lipase addition, cell density at harvest and ARA levels in the polar lipid of the harvested biomass. In experiment B012, the lipases were omitted from the culture, backpressure was set to 15 psi and airflow at 12, to provide about 10 ppm dissolved oxygen during culturing. The cell density (OD600) of the inoculum was 9.19, so 200 mL was added to the 25 L medium in the fermenter to achieve a starting OD600 calculated at 0.08. The ARA oil and FFA were added as before. The pH dropped from an initial 7.08 at 0 h to 4.63 at 15.68 h but then started to increase in the last 30 min of the culturing. At this point, the culture might have reached stationary phase and glucose was depleted. After the exhaustion of glucose, the cells might have started breaking down phospholipids for maintenance. It was therefore considered important to harvest the culture before it reached stationary phase. The optical density, calculated at TO and corrected by washing the cells with water at 16 h, increased from 0.08 to 27.4 at 16 h, yielding a culture density of 9 g/L on a dry weight basis.

The cell biomass was harvested from the culture and the pellets washed twice with cold deionized water. The washed cells were heat inactivated at a temperature of approximately 95° C. for 3 min, then chilled by immersing the container in a water bath with ice. The heat inactivation of the yeast cells was successful as shown by a lack of viable cells when plated. In this experiment, 225 g of dry cell biomass was generated. Total lipid was extracted from biomass samples and analysed as before. The freeze-dried cells contained about 4.7% crude lipid. The polar lipid fraction from this experiment had 4.1% ARA and the TAG fraction had 4.0% ARA as a percentage of the total fatty acid content of those fractions (Table 31). The total lipid also had less TAG, MAG and FFA than in previous experiments, as shown by TLC. This was taken as an indication that the cells took up the ARA and incorporated it into PL in cell membranes under the prescribed culture conditions, however, the PL might have been broken down to some extent to maintain cellular activities due to glucose depletion in the medium.

Another experiment (B013) was carried out with the following adjustments to the culture conditions: the starter culture OD600 was between 4 and 5, the ARA FFA and the ARA oil were formulated with 5% Triton X-100 as a concentrated pre-mix and then added to the fermenter prior to inoculation, the pH trend was used to estimate the optimal harvest point by monitoring it to be greater than 4.0. The pH trend was closely monitored from 14 h to ensure culture termination and cell harvest before glucose exhaustion occurred and the pH started to rise. To make the culture medium for this experiment, 50 mL Triton X-100 was dissolved in 1 L deionized water and autoclaved. The Triton X-100 separated from the water as the sterilised solution cooled overnight and needed to be warmed to about 50° C. to re-dissolve it, with shaking. Once the Triton X-100 was fully dissolved, it was vigorously mixed with 10.0 g ARA and 100 mL ARA oil to form an emulsion and then pumped into the fermenter. Lastly, 400 mL inoculum culture was transferred to the fermenter by overpressure. The calculated culture density (OD600) at inoculation was 0.07.

During the culturing, the dissolved oxygen level dropped to zero at 6 h post inoculation under the initial set up conditions of airflow at 10 L/m, pressure 10 psi and DO 15.9. The temperature gradually dropped from 28° C. to 23° C. overnight as the culture density was insufficient to generate heat. The reduced temperature was likely beneficial in decreasing the culture growth rate shown by the gradual decrease in pH decline. At 14 h, the airflow, stirring rate and backpressure were changed to increase the DO and the temperature was also increased. The OD600 was 7.4 at 14 h, therefore, the fermentation was extended by 2 hours until the OD600 was above 10 and the pH began to stabilise at pH 5. The culture was run without pH control for 16 hours, the pH naturally falling from pH 6.96 to 5.07 due to acid production from cellular metabolism. The cell density (dry weight) was 5.27 g/L at 16 h, while the OD600 increased from 0.07 to 12.1 at 16 h. The culture assimilated 4.5 g/L of glucose, which was 51% of the 8.9 g/L glucose supplied in the start medium.

The harvested cells were heat inactivated at a temperature of 95° C. for 3 min as before, yielding 584 g wet weight of biomass corresponding to 114 g dry weight. Lipid was extracted from freeze-dried samples and analysed as before. The total lipid content of the 16 h freeze-dried cells was 3.4%. The TLC analysis showed that more polar lipid was present than in experiment B012. The ARA level in the polar lipid and TAG fractions were 10.2% and 13.3%, respectively. The parameters for Experiments B005, B009, B012 and B013 are provided in Table 30. The data for the fatty acid compositions are provided in Table 31.

TABLE 30
Comparison of Y. lipolytica W29 cultures
under different fermentation conditions

| Experiment | B005 | B009 | B012 | B013 |
|---|---|---|---|---|
| Base medium (g/L) | YPD | YPD | YPD | YPD |
| Lipases | 0 | 3 lipases | 0 | 0 |
| TritonX-100 (mL/ L) | 2 | 2 | 2 | 2 |
| Ara oil (mL/L) | 4 | 4 | 4 | 4 |
| Ara-FFA (g/25 L) | 12.5 | 10 | 10 | 10 |
| OD600 of inoculum | 4.2 | 13.3 | 9.2 | 9.2 |
| Starting OD600 | 0.07 | 0.1 | 0.08 | 0.08 |
| Harvest time (h) | 15.5 | 16 | 16 | 16 |
| Start pH | 7.01 | 6.7 | 7.08 | 7.08 |
| Harvest pH | 5.8 | 3.5 | 5.11 | 5.11 |
| Backpressure (psi) | 11 | 15 | 10 | 10 |
| Stirrer (%) | 5 | 30 | 30 | 30 |
| Air Flow | 10 | 20 | 15 | 15 |
| Dissolved Oxygen | 7.92 | 6.29 | 19.13 | 19.13 |
| Biomass (g dry weight) | 75 | 260 | 220 | 220 |
| ARA (%) in polar lipid | 16 | 2.5 | 4.1 | 10.2 |

It was concluded that experiment B013 had provided a useful biomass content and a reasonable level of ARA incorporation into polar lipid, even though further optimisation of both parameters was desired. The cell biomass produced in B013 and lipids extracted from these cells were used in Maillard reactions simulating food preparations as described in Example 7 below.

TABLE 31
Fatty acid composition of polar lipids and
TAG in Y. lipolytica after culturing with ARA,
for experiments B005, B009, B012 and B013.

| | B005 | | B012 | | B013 | |
|---|---|---|---|---|---|---|
| | Polar | | Polar | | Polar | |
| | lipid | TAG | lipid | TAG | lipid | TAG |
| C14:0 | 0.2 | 0.0 | 0.0 | 0.0 | 0.2 | 0.0 |
| C15:0 | 0.0 | 0.0 | 0.2 | 0.2 | 0.8 | 0.0 |
| C16:0 | 12.7 | 14.1 | 10.1 | 7.5 | 12.0 | 8.0 |
| C16:1Δ9 | 7.4 | 0.0 | 7.7 | 9.3 | 9.3 | 5.6 |
| C18:0 | 0.5 | 11.3 | 1.6 | 0.3 | 0.8 | 5.6 |
| C18:1Δ9 | 32.7 | 35.7 | 50.6 | 48.5 | 29.2 | 35.7 |
| C18:1Δ11 | 0.4 | 0.0 | 0.6 | 0.5 | 0.5 | 0.9 |
| C18:2 (LA) | 25.0 | 16.7 | 20.8 | 24.3 | 33.5 | 17.1 |
| C18:3ω3 (ALA) | 0.2 | 0.0 | 0.0 | 0.0 | 0.0 | 0.0 |
| C18:3ω6 (GLA) | 1.6 | 0.0 | 0.4 | 0.5 | 0.4 | 0.0 |
| C19:0 | 0.0 | 0.0 | 0.4 | 0.9 | 0.8 | 0.0 |
| C20:0 | 0.0 | 0.0 | 0.3 | 0.4 | 0.4 | 0.0 |
| C20:1Δ11 | 0.3 | 0.0 | 0.0 | 0.0 | 0.0 | 0.0 |
| C20:1Δ5 | 0.4 | 0.0 | 0.0 | 0.0 | 0.0 | 0.0 |
| C20:2ω6 (EDA) | 0.2 | 0.0 | 0.0 | 0.0 | 0.0 | 0.0 |
| C20:3ω6 (DGLA) | 1.9 | 0.0 | 0.6 | 0.5 | 0.7 | 0.6 |
| C20:4ω6 (ARA) | 16.4 | 22.1 | 4.1 | 4.0 | 10.2 | 13.3 |
| C24:0 | 0.2 | 0.0 | 1.0 | 2.8 | 1.2 | 12.9 |
| % of DCW | 2.0 | 0.1 | | | | |

### Example 7. Maillard Reaction and Volatiles Test

As described in Example 5 and 6, polar lipids including PL with one or more of the ω6 fatty acids GLA, DGLA, ARA, DTA or DPA-ω6 are produced in yeast cells, extracted and purified. In an initial experiment to see if a Maillard reaction could be induced and what properties the resultant products would have, polar lipid preparations including GLA or ARA were mixed with cysteine and ribose in glass vials and heated in an oven at 140° C. for 1 h. This Example describes these experiments and the results.

The Maillard reaction is a chemical reaction between a reducing sugar and an amino group, for example in a free amino acid, with application of heat. Like caramelisation, it is a form of non-enzymatic browning. In this reaction, the amino group reacts with a carbonyl group of the sugar and produces N-substituted glycosylamine and water. The unstable glycosylamine undergoes a reaction called an Amadori rearrangement and produces ketosamines. The ketosamines can react further in different ways to produce reductones, diacetyl, aspirin, pyruvaldehyde, and other short-chain hydrolytic fission products. Finally, a furan derivate may be obtained which reacts with other components to polymerize into a dark-coloured insoluble material containing nitrogen.

The outcome of the Maillard reaction depends on temperature, time and pH. For example, the reaction slows at low temperature, low pH and low water activity (Aw) levels. The browning colour occurs more quickly in alkaline conditions because the amino group remains in the basic form. The reaction peaks at intermediate water activities such as Aw of 0.6-0.7. In addition to colour, many volatile aroma compounds are typically formed during the Maillard reaction. Flavour-intensive compounds may be formed in the presence of the sulphur-containing amino acids methionine or cysteine or other sulphur containing compounds such as thiamine. Unsaturated fatty acids and aldehydes formed from fatty acids also contribute to the formation of heterocyclic flavour compounds during the Maillard reaction (Feiner, 2006).

### Experiment 1

### Preparation of Polar Lipid Samples

Y. lipolytica strain W29 was grown in 200 ml of YPD medium containing either GLA (Catalog No. U-63-A, NuChek Prep Inc., USA) or ARA (Cat. No. U-71-A, NuChek Prep Inc, USA) by adding 100 mg of the fatty acids dissolved in 300 μl ethanol to the medium. For a “non-fed” control, 300 μl of ethanol without fatty acid was added to a corresponding culture of W29. The starting OD600 of each culture was 0.1. The cultures were grown at 28° C. with shaking at 250 rpm for 2 days and the cells harvested by centrifugation at 4,600 g for 15 min. The cell pellets were washed twice with 10 ml water, with vortexing for 2 min and centrifugation for 15 min with removal of the supernatant each time. The pellets were then freeze dried and weighed, yielding 663 mg of the GLA-fed cells, 898 mg of the ARA fed cells and 962 mg of the control cells. Lipid was extracted from the cells and fractionated into TAG and polar lipid fractions using TLC as described in Example 1 and stored at −20° C. Polar lipid was also extracted from 10 g of pork meat as described in Example 2.

Samples of the extracted polar lipids were methylated and the FAME analysed by GC as described in Example 1. This also provided the amount of polar lipid in the extracts. Table 32 provides the fatty acid composition of the polar lipids, expressed for each fatty acid as a weight percentage of the total fatty acid content for each isolated polar lipid fraction. Notably, the polar lipid fractions from the GLA- and ARA-fed cells had 61.3% GLA and 18.2% ARA, respectively. The polar lipid from the pork meat had 6.6% ARA in its total fatty acid content, whereas the TAG from pork had very little ARA, consistent with the data in Example 2. The polar lipid from pork also had about 14% C18:0 (stearic acid), much higher than in the polar lipids from Y. lipolytica, and detectable amounts of the ω3 fatty acids ALA, DPA and DHA. The pork polar lipid fraction of this experiment 1 also contained 0.5% C20:2ω6, 0.3% C22:1, 0.8% C22:4ω6+C22:3ω3 (not resolved), 0.3% C24:1, 0.7% C22:5ω3 (DPA) and 0.2% C22:6ω3 (DHA), not listed in Table 32. These fatty acids were not detected in the Y. lipolytica polar lipid fractions used in this experiment 1.

### Maillard Reactions

Samples of 8.0 mg of polar lipid from the ARA-fed cells, 7.6 mg from the GLA-fed cells, 9.0 mg from the control cells and 16.0 mg of polar lipid from the pork meat, each dissolved in chloroform, were transferred to 20 ml glass vials. The solvent was evaporated under nitrogen flow at room temperature. 2 ml of 0.1 M potassium phosphate buffer, pH 7.2, containing 4.5 mg/ml ribose (Catalog No. R9629, Sigma-Aldrich) and 5.0 mg/ml cysteine (Catalog No. 30089, Sigma-Aldrich) was added to each vial, and the vials tightly closed with metal lids having PTTF liners. A control vial had the buffer but no polar lipid. The vials were subjected to ultrasonication in a water bath at 40° C. for 1 h and then heated in an oven at 140° C. for 1 h by placing the vials on the bottom metal surface of the oven. After the heating, the mixtures all appeared orange-brown in colour, suggesting that a chemical reaction had occurred. Serendipitously, the vial containing the polar lipid from the ARA-fed cells leaked and a distinct roast meat-like aroma was noticed that spread inside and even outside the laboratory. The other vials were then cooled, opened and smelled. The heated mixtures having the ARA-fed and pork polar lipids gave off pleasant, meat-like aromas, while the mixture including the GLA-fed polar lipid had a mild garlic-like aroma. In contrast, the mixture having the polar lipid from Y. lipolytica that had not been fed the amino acids (control) and the control mixture lacking lipid emitted a sulphurous aroma. The inventors concluded that the polar lipid containing ARA provided a more meat-like aroma than the polar lipid containing GLA, even though the GLA was present at a 3-fold greater amount than the ARA. The inventors also concluded that the presence of ARA in the polar lipid provided the meat-like aroma, which did not occur with the corresponding polar lipid lacking the ARA.

These observations prompted the inventors to carry out further tests with extracted lipids containing ω6 fatty acids to determine their capacity to provide meat-like flavour and aroma compounds and to measure the volatiles by GC-MS, as follows. Experiments were also carried out with whole cells having the ω6 fatty acids in their polar lipids, rather than with extracted lipids from the cells, as follows.

### Experiment 2

Encouraged by the results of the first experiment, a second experiment was performed, including a sensory evaluation by a panel of volunteers to detect aromas. Y. lipolytica strain W29 was grown in a total of 3 L culture medium with ARA added to the medium as for Experiment 1. Polar lipid was extracted from the cells as before, see experiment 3 of Example 5. The fatty acid composition of the polar lipid was determined by GC-FID of FAME, showing the presence of 16.3% ARA (Table 32, 3 L). Samples of 15 mg of polar lipid were treated in the same manner as in Experiment 1. Additional, control mixtures having buffer with ribose but without the cysteine were prepared to test the effect of omitting the sulphur-containing amino acid. Other mixtures were prepared including either soy lecithin (The Ingredients Centre, VIC, Australia) or an ARA-containing oil (Jinan Boss Chemical Industry Co, China) containing 50% ARA as a percentage of the total fatty acid content (Example 5). As before, 2.0 ml of 0.1 M potassium phosphate buffer, pH 7.2, containing 4.5 mg/ml ribose and in some cases 5.0 mg/ml cysteine was added to each 10 mL SPME vial, and the vials tightly closed with PTFE-lined screw top caps. The vials were then subjected to ultrasonication for 1 h in a water bath at 40° C. and heated at 140° C. for 1 h, as before. After the heat treatment, the mixtures having ribose without cysteine had a dark brown, coffee-like colour, whereas those having both ribose and cysteine were lighter brown in colour.

After the vials were cooled to room temperature, sensory analysis was carried out by nine volunteers, consisting of 5 males and 4 females aged 30 to 65 years, of different backgrounds. The sample identities were not revealed until after the completion of the sensory evaluation. Each vial was gently shaken and the lid was opened to sniff the aroma. The vials containing the lipid and ribose without cysteine were presented first, followed by the vials containing the lipid, ribose and cysteine, in the order vials 1, 4, 6, 2, 5, 7 and 3. The volunteers' reactions were recorded (Table 33). It was clear that although there was some diversity in the responses, vial 3 was consistently referred to as providing a pleasant, meaty or roast beef aroma.

The samples were then analysed by HS-SPME-GCMS for volatiles as described in Example 1. The GC-MS analyses revealed volatile compounds which were present in the mixtures containing the ARA-fed polar lipid but absent from the mixtures containing the polar lipid from the non-fed cells. These compounds were: 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; and 2,3,4,5-Tetramethylcyclopent-2-en-1-ol. It was concluded that these compounds were associated with the roasted meat-like aroma for the mixture in vial 3.

In a repeat of the experiment, the amounts of ribose and cysteine were halved, attempting to reduce sulphurous aromas. Similar results were obtained as before, with some reduction in the sulphurous component of the aromas. The responses from 6 other volunteers confirmed that the polar lipid from the ARA-fed Y. lipolytica provided roasted beef-like aromas, different to the aromas from the soy lecithin and ARA oil mixtures. Notably, one of the volunteers had a pet dog which showed great interest in the aroma.

TABLE 32
Fatty acid composition of polar lipids extracted from Y. lipolytica cultures fed with GLA, ARA or no added fatty acid (control).

| | | | | | | | | | |
|---|---|---|---|---|---|---|---|---|---|
| | | | | C16: | C16: | | | | C18: |
| Sample | C14:0 | C15:0 | C16:0 | 1Δ7 | 1Δ9 | C17:1 | C18:0 | C18:1 | 1Δ11 |

Experiment 1

| | | | | | | | | | |
|---|---|---|---|---|---|---|---|---|---|
| Control | 0.2 | 0.3 | 9.4 | 4.0 | 10.4 | 1.8 | 0.8 | 49.9 | 0.3 |
| GLA-fed polar lipid | 0.1 | 2.0 | 17.9 | 0.1 | 2.0 | 0.6 | 2.9 | 8.5 | 0.1 |
| ARA-fed polar lipid | 0.3 | 1.0 | 14.1 | 0.5 | 9.5 | 1.6 | 1.1 | 42.7 | 0.5 |
| Pork polar lipid | 0.2 | 6.6 | 19.0 | 0.6 | 0.9 | 0.6 | 14.0 | 16.7 | 4.4 |
| Pork TAG | 1.9 | 0.0 | 28.0 | 0.0 | 2.7 | 0.3 | 14.9 | 41.2 | 4.8 |

Experiments 2-4

| | | | | | | | | | |
|---|---|---|---|---|---|---|---|---|---|
| ARA-fed polar lipid (1 L) | 0.2 | 0.8 | 15.6 | 0.8 | 8.6 | 0.2 | 1.4 | 46.9 | 0.4 |
| ARA-fed polar lipid (3 L) | 0.0 | 0.0 | 15.5 | 0.9 | 8.4 | 0.6 | 1.7 | 44.8 | 0.4 |
| Soy lecithin (unpurified) | 0.1 | 0.0 | 20.5 | 0.0 | 0.1 | 0.0 | 4.3 | 8.4 | 1.4 |
| Soy lecithin (TLC pure) | 0.1 | 0.0 | 20.9 | 0.0 | 0.1 | 0.0 | 4.0 | 9.0 | 1.4 |

Experiment 6

| | | | | | | | | | |
|---|---|---|---|---|---|---|---|---|---|
| ARA-fed polar lipid | 0.2 | 0.2 | 14.7 | 2.9 | 0.9 | 1.0 | 0.7 | 28.0 | 0.7 |
| ARA-fed free fatty acid | 0.1 | 0.1 | 6.8 | 0.4 | 0.2 | 0.1 | 53.1 | 4.8 | 0.2 |

Experiment 7

| | | | | | | | | | |
|---|---|---|---|---|---|---|---|---|---|
| GLA-fed polar lipid (3 L) | 0.0 | 0.0 | 16.2 | 0.4 | 7.1 | 0.0 | 2.4 | 19.5 | 0.2 |

Experiment 8

| | | | | | | | | |
|---|---|---|---|---|---|---|---|---|
| ARA-fed whole cells | 0.2 | 0.0 | 12.7 | 7.4 | 0.0 | 0.5 | 32.7 | 0.4 |

Fatty acid composition of polar lipids extracted from Y. lipolytica cultures fed with GLA, ARA or no added fatty acid (control).

| | | | | | | | | |
|---|---|---|---|---|---|---|---|---|
| | | C18:3 | C18:3 | | C20:3 | C20:4 | | |
| | C18:2 | ω6 | ω3 | | ω6 | ω6 | | |
| Sample | (LA) | (GLA) | (ALA) | C20:0 | (DGLA) | (ARA) | C22:0 | C24:0 |

Experiment 1

| | | | | | | | | |
|---|---|---|---|---|---|---|---|---|
| Control | 22.8 | 0.0 | 0.0 | 0.0 | 0.0 | 0.0 | 0.0 | 0.1 |
| GLA-fed polar lipid | 3.2 | 61.3 | 0.0 | 0.1 | 0.6 | 0.3 | 0.0 | 0.3 |
| ARA-fed polar lipid | 9.8 | 0.0 | 0.0 | 0.0 | 0.6 | 18.2 | 0.0 | 0.2 |
| Pork polar lipid | 25.2 | 0.3 | 0.5 | 0.2 | 0.8 | 6.6 | 0.3 | 0.2 |
| Pork TAG | 5.1 | 0.1 | 0.3 | 0.2 | 0.0 | 0.1 | 0.0 | 0.0 |

Experiments 2-4

| | | | | | | | | |
|---|---|---|---|---|---|---|---|---|
| ARA-fed polar lipid (1 L) | 9.1 | 1.4 | 0.0 | 0.0 | 0.3 | 14.1 | 0.0 | 0.2 |
| ARA-fed polar lipid (3 L) | 9.6 | 1.2 | 0.0 | 0.0 | 0.3 | 16.3 | 0.0 | 0.2 |
| Soy lecithin (unpurified) | 57.5 | 0.0 | 6.8 | 0.1 | 0.0 | 0.0 | 0.4 | 0.3 |
| Soy lecithin (TLC pure) | 56.5 | 0.0 | 6.9 | 0.2 | 0.0 | 0.0 | 0.6 | 0.4 |

Experiment 6

| | | | | | | | | |
|---|---|---|---|---|---|---|---|---|
| ARA-fed polar lipid | 44.6 | 2.1 | 0.0 | 0.0 | 0.8 | 3.2 | 0.1 | 0.1 |
| ARA-fed free fatty acid | 4.6 | 0.5 | 0.0 | 0.0 | 1.3 | 7.3 | 12.6 | 8.1 |

Experiment 7

| | | | | | | | | |
|---|---|---|---|---|---|---|---|---|
| GLA-fed polar lipid (3 L) | 1.6 | 51.8 | 0.0 | 0.1 | 0.5 | 0.0 | 0.0 | 0.2 |

Experiment 8

| | | | | | | | | |
|---|---|---|---|---|---|---|---|---|
| ARA-fed whole cells | 25.0 | 1.4 | 0.2 | 0.0 | 1.9 | 16.4 | 0.0 | 0.2 |

The polar lipid fraction of Experiment 8 also contained 0.3% C20:1Δ11, 0.4% C20:1Δ5 and 0.2% C20:2ω6.

TABLE 33
Aromas of mixtures of polar lipid, ribose and cysteine after heat
treatment, as detected by a sensory panel of 9 volunteers.

| Vial | | Ribose | Cysteine | |
|---|---|---|---|---|
| No. | Lipid source | (mg/ml) | (mg/ml) | Description of aromas |
| 1 | Control (non-fed) | 4.5 | 0 | caramel-like, baked, |
| | polar lipid | | | biscuity |
| 2 | Control (non-fed) | 4.5 | 5.0 | smoky, garlic, burnt |
| | polar lipid | | | |
| 3 | ARA-fed polar | 4.5 | 5.0 | Roasted beef, meaty, |
| | lipid | | | pleasant |
| 4 | Soy lecithin | 4.5 | 0 | baked, fishy |
| 5 | Soy lecithin | 4.5 | 5.0 | smoky, garlic, burnt |
| | | | | popcorn |
| 6 | ARA oil (TAG) | 4.5 | 0 | something raw, raw fish |
| 7 | ARA oil (TAG) | 4.5 | 5.0 | lighter aroma, old beef |
| | | | | roast |

### Experiment 3

In another experiment, 15 mg samples of the extracted polar lipid preparations, the soy lecithin or the ARA oil were separately mixed with 2 ml of the potassium phosphate buffer containing 2.25 mg/ml ribose and 2.5 mg/ml cysteine, pH 7.2, in 12 ml glass tubes rather than the SPME vials. The fatty acid compositions for the Y. lipolytica-derived preparations and the soy lecithin (unpurified and TLC-purified) are provided in Table 32, 3L. The lipids tested were:
1. Polar lipid from Y. lipolytica grown in the presence of ARA (Yl ARA)
2. Polar lipid from Y. lipolytica grown in the absence of ARA (Yl) (Control)
3. Soy lecithin (The Ingredients Centre)
4. ARA oil (Jinan Boss Chemical Industry Co, China)
5. No lipid (control)

In an initial attempt, the mixtures were sonicated in the 12 ml Pyrex glass tubes with plastic caps lined with PTFE seals and then heated for 1 h at 140° C. The tubes were placed in a rack in the oven rather than in contact with a metal surface of the oven. This time, the mixtures were not brownish in colour, instead appeared rather turbid but colourless. GC-MS analysis showed only low levels of volatiles, indicating that the Maillard reactions had not gone to completion. The inventors thought that insufficient heating or the smaller surface area of the mixtures in the tubes may have contributed to the reduced reaction. The remaining mixtures were therefore transferred to SPME vials and heated again at 140° C. for 2 h by placing the vials on an aluminium foil inside the oven. This time, the colour of the mixtures changed to pale brown as in previous experiments. The vials were cooled down and stored at −20° C. For GC-MS analysis of volatile compounds, 0.5 ml of each sample was transferred into new SPME vials for injection in the split 1:20 mode and other 0.2 ml transferred into new vials for injection by splitless mode.

### Volatile Compounds Released by the Treatment

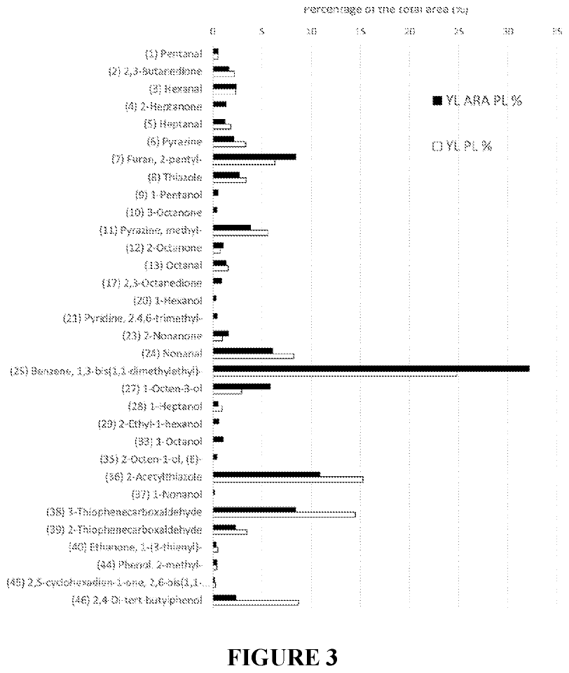

The profile of volatile compounds released by heating the extracted lipids with the mixture of ribose and cysteine was evaluated by headspace solid-phase microextraction coupled with gas chromatography-mass spectrometry (HS-SPME-GCMS) as described in Example 1. The majority of volatiles are generated by a combination of lipid oxidation and other degradation processes, as well as Strecker reaction and Maillard reaction products, including production of aldehydes, alcohols, ketones, pyrazines and furans. The GC-MS data are shown in Table 34 and FIG. 3 which presents the levels of each of the identified compounds as the area percentage (%) of total identified compounds for reaction mixtures containing the ARA-polar lipid (YL ARA) or non-fed polar lipid (YL).

The sample containing polar lipid from the Y. lipolytica cells fed with ARA, heated in the presence of cysteine and ribose under conditions to produce the Maillard reaction, produced specific volatile compounds including 2-heptanone, 3-octanone, 2,3-octanedione, 1-pentanol, 1-hexanol, 2-ethyl-1-hexanol, 1-octanol, trans-2-octen-1-ol and 1-nonanol. These compounds were not detected in the Maillard products from the polar lipid extracts from the control Y. lipolytica cells grown in the absence of ARA (YL). Of these, 3-octanone and 1-nonanol were detected only in the reaction having the YL ARA polar lipid i.e. not in any of the other vials. Other compounds, namely 2-heptanone, 2,3-octanedione, 1-hexanol and 1-octanol were detected only in the reactions having YL ARA and the soy lecithin. The ω6 fatty acid in the reactions with polar lipid containing ARA clearly created a chemical difference which was associated with the sensory difference observed by the volunteers, with an increased amount of lipid oxidation products and reduced amounts of heterocyclic compounds, such as pyrazines. The presence of certain ketone and alcohol compounds registered here were also observed in volatile profiles for meat flavours as a result of lipid oxidation. The ketone 2-heptanone present in the samples with YL ARA and soy lecithin was thought to be due to lipid oxidation and related to ethereal, butter or spicy flavours. The volatile compound 1,3-bis(1,1-dimethylethyl)-benzene was the main compound produced (FIG. 3) and was significantly increased in amount in the reaction mixture made with the ARA-polar lipid relative to the control polar lipid from Y. lipolytica cells. That compound has a characteristic beef-like aroma.

Results from the experiment indicated that the compound acetylthiazole, common to all samples tested and shown in Table 34, has a sulphurous and roast meat aroma resulted from the reaction with cysteine and ribose. The aldehydes hexanal and nonanal, which were produced from all mixtures except the ‘no lipid’ control sample, were produced from the lipid oxidation. Hexanal is associated with oxidation of ω6 fatty acids such as LA and ARA. Nonanal contributes to tallow and fruity flavour and it is one of the key volatiles in cooked beef together with octanal. Octanal was produced from the samples containing YL ARA, YL and soy lecithin, i.e. all three polar lipid samples, but not produced from the ARA-Oil and no lipid samples. The unsaturated alcohol 1-octen-3-ol, also produced in all the oil-containing samples tested (YL ARA, YL, soy lecithin and ARA Oil) may contribute to an herbaceous aroma resulted from thermal decomposition of methyl linoleate hydroperoxide. The compound 2-pentylfuran, present in all but the no lipid mixture, was derived from LA. Furan-containing compounds were also possibly produced from the thermal degradation of sugars.

TABLE 34
Detection and identification by HS-SPME-GC-MS of volatile compounds produced in Maillard reactions with
Y. lipolytica polar lipids after feeding the cells with ARA (YL ARA) or not fed ARA as a control (YL),
indicating the detected presence (Y) or absence (N) of the compounds in each mixture. LRI: linear retention
index for polar columns obtained from literature and standards (S); observed LRI: linear retention indexcalculated from
tested samples; ID: methods of identification; MS: mass spectrum; RI: retention index; S: standard mass spectrum.

| | | Reported | Observed | | YL | | Soy | ARA | No |
|---|---|---|---|---|---|---|---|---|---|
| No. | Compound | LRI | LRI | ID | ARA | YL | lecithin | oil | lipid |
| 1 | Pentanal | 990 | 990 | MS, RI, S | Y | Y | Y | N | N |
| 2 | 2,3-Butanedione | 986 | 991-992 | MS, RI | Y | Y | Y | Y | Y |
| 3 | Hexanal | 1078 | 1075- | MS, RI, S | Y | Y | Y | Y | N |
| | | | 1076 | | | | | | |
| 4 | 2-Heptanone | 1185 | 1181- | MS, RI | Y | N | Y | N | N |
| | | | 1182 | | | | | | |
| 5 | Heptanal | 1188 | 1183 | MS, RI, S | Y | Y | N | N | N |
| 6 | Pyrazine | 1209 | 1214- | MS, RI | Y | Y | Y | N | Y |
| | | | 1220 | | | | | | |
| 7 | Furan, 2-pentyl- | 1236 | 1229- | MS, RI, S | Y | Y | Y | Y | N |
| | | | 1232 | | | | | | |
| 8 | Thiazole | 1210- | 1251- | MS, RI | Y | Y | Y | Y | Y |
| | | 1270 | 1257 | | | | | | |
| 9 | 1-Pentanol | 1254 | 1256 | MS, RI, S | Y | N | N | Y | N |
| 10 | 3-Octanone | 1251 | 1256 | MS, RI | Y | N | N | N | N |
| 11 | Pyrazine, methyl- | 1238- | 1266- | MS, RI | Y | Y | Y | Y | Y |
| | | 1309 | 1276 | | | | | | |
| 12 | 2-Octanone | 1280 | 1280- | MS, RI | Y | Y | Y | Y | N |
| | | | 1287 | | | | | | |
| 13 | Octanal | 1294 | 1290- | MS, RI, S | Y | Y | Y | N | N |
| | | | 1291 | | | | | | |
| 14 | 2-Propanone, 1- | 1266- | 1311- | MS, RI | N | N | N | Y | Y |
| | hydroxy- | 1340 | 1322 | | | | | | |
| 15 | 2-Heptenal, (Z)- | 1320 | 1326 | MS, RI | N | N | Y | N | N |
| 16 | Pyrazine, 2,6-dimethyl- | 1325 | 1328- | MS, RI | N | N | Y | N | Y |
| | | | 1331 | | | | | | |
| 17 | 2,3-Octanedione | 1320- | 1330- | MS, RI | Y | N | Y | N | N |
| | | 1376 | 1331 | | | | | | |
| 18 | Pyrazine, ethyl- | 1292- | 1334- | MS, RI | N | N | Y | Y | Y |
| | | 1359 | 1337 | | | | | | |
| 19 | Pyrazine, 2,3-dimethyl- | 1345 | 1346 | MS, RI | N | N | Y | N | N |
| 20 | 1-Hexanol | 1354 | 1357 | MS, RI | Y | N | Y | N | N |
| 21 | Pyridine, 2,4,6- | 1369 | 1367 | MS, RI, S | Y | N | N | N | N |
| | trimethyl- | | | | | | | | |
| 22 | Pyrazine, 2-ethyl-6- | 1353- | 1384 | MS, RI | N | N | Y | N | N |
| | methyl- | 1420 | | | | | | | |
| 23 | 2-Nonanone | 1394 | 1391 | MS, RI | Y | Y | Y | N | N |
| 24 | Nonanal | 1399 | 1396 | MS, RI, S | Y | Y | Y | Y | N |
| 25 | Benzene, 1,3-bis(1,1- | 1420- | 1429- | MS, RI | Y | Y | N | N | N |
| | dimethylethyl)- | 1454 | 1431 | | | | | | |
| 26 | 2-Octenal, (E)- | 1400- | 1427- | MS, RI | N | N | Y | Y | N |
| | | 1441 | 1432 | | | | | | |
| 27 | 1-Octen-3-ol | 1456 | 1455- | MS, RI, S | Y | Y | Y | Y | N |
| | | | 1457 | | | | | | |
| 28 | 1-Heptanol | 1460 | 1460 | MS, RI | Y | Y | N | N | N |
| 29 | 2-Ethyl-1-hexanol | 1492 | 1495 | MS, RI | Y | N | N | N | Y |
| 30 | 2-Decanone | 1484 | 1499 | MS, RI | N | N | Y | N | N |
| 31 | Decanal | 1502 | 1503 | MS, RI | N | N | Y | N | N |
| 32 | trans-3-Nonen-2-one | 1495- | 1516 | MS, RI | N | N | Y | N | N |
| | | 1547 | | | | | | | |
| 33 | 1-Octanol | 1561 | 1562 | MS, RI | Y | N | Y | N | N |
| 34 | Pyridine, 2-pentyl- | 1527- | 1573 | MS, RI | N | N | Y | N | N |
| | | 1592 | | | | | | | |
| 35 | 2-Octen-1-ol, (E)- | 1610- | 1615 = 1620 | MS, RI | Y | N | Y | Y | N |
| | | 1645 | | | | | | | |
| 36 | 2-Acetylthiazole | 1661 | 1650- | MS, RI | Y | Y | Y | Y | Y |
| | | | 1651 | | | | | | |
| 37 | 1-Nonanol | 1668 | 1665 | MS, RI | Y | N | N | N | N |
| 38 | 3- | 1653- | 1683- | MS, RI | Y | Y | Y | Y | Y |
| | Thiophenecarboxaldehyde | 1693 | 1685 | | | | | | |
| 39 | 2- | 1655- | 1698- | MS, RI | Y | Y | Y | Y | Y |
| | Thiophenecarboxaldehyde | 1734 | 1700 | | | | | | |
| 40 | Ethanone, 1-(3-thienyl)- | 1725- | 1779- | MS, RI | Y | Y | N | N | N |
| | | 1785 | 1780 | | | | | | |
| 41 | Ethanone, 1-(2-thienyl)- | 1735- | 1771- | MS, RI | N | N | Y | Y | Y |
| | | 1785 | 1773 | | | | | | |
| 42 | 2,4-Decadienal, (E,E)- | 1790- | 1811 | MS, RI | N | N | N | Y | N |
| | | 1821 | | | | | | | |
| 43 | 3-Methyl-2- | 1761- | 1815 | MS, RI | N | N | N | N | Y |
| | thiophenecarboxaldehyde | 1815 | | | | | | | |
| 44 | Phenol, 2-methyl- | 1992- | 2012 | MS, RI | Y | Y | N | N | N |
| | | 2025 | | | | | | | |
| 45 | 2,5-cyclohexadien-1- | 2094- | 2107 | MS, RI | Y | Y | N | N | N |
| | one, 2,6-bis(1,1- | 2117 | | | | | | | |
| | dimethylethyl)-4- | | | | | | | | |
| | hydroxy-4-methyl- | | | | | | | | |
| 46 | 2,4-Di-tert-butylphenol | 2280- | 2321- | MS, RI | Y | Y | Y | Y | Y |
| | | 2327 | 2322 | | | | | | |

### Experiment 4. Optimisation of the Amount of Lipid in the Reactions

An experiment was carried out varying the amount of lipid used in the Maillard reactions, to test whether smaller amounts of polar lipid could be heat treated and the reaction products still be detected by GC-MS. The purpose of the experiment is to define optimal amounts which produce a chromatogram that detects most of the compounds at the same time that produces maximum overall intensity. Samples containing 0.5, 2.5, 5.0 or 7.5 mg of 18:0/18:1-phosphatidylcholine (Catalog No. 850467C, Avanti Polar Lipids) in chloroform were transferred to 20 ml SPME vials. Aliquots of 2.5 or 7.5 mg of soy lecithin powder, or 2.5 or 7.5 mg polar lipid extracted from the soy lecithin powder by TLC as described in Example 1, were also transferred to SPME vials. The fatty acid composition of the unpurified and TLC-purified soy lecithin is provided in Table 32; the purification had little effect on the fatty acid composition. After evaporation of the chloroform under a flow of nitrogen, 1 ml of 0.2 M potassium phosphate buffer, pH 7.2, containing 2.25 mg/ml ribose and 2.5 mg/ml cysteine were added to the vials and the lids were tightly closed. The vials were subjected to ultrasonication for 1 h at 40° C. in a water bath to emulsify the mixtures and then incubated at 140° C. for 1 h by placing the vials on aluminium foil inside an oven. After the vials were cooled, the volatile compounds in the headspace of each vial were analysed by solid-phase microextraction coupled with gas chromatography-mass spectrometry (HS-SPME-GCMS) as before. The reaction mixtures including 0.5 mg lipid showed peaks for the volatile compounds but at lower intensities than desired with some compounds being undetected. Intermediate lipid amounts (2.5 and 5.0 mg) showed an increased response for most of the compounds, while the maximum amount tested (7.5 mg) of polar lipid showed an overall reduction in intensities perhaps due to overloading. Therefore, the mixtures having 2.5 mg polar lipid in 1 ml reaction volume showed optimal performance without suffering from either disadvantage. That amount of polar lipid was considered optimum for future experiments. Mixtures having 5.0 mg polar lipid in 1 ml reaction volume were also considered suitable for the analyses.

A comparison of reaction products in the mixtures having soy lecithin, either purified through TLC or not purified, revealed the presence of several hydrocarbon compounds in the reaction mixture having the purified soy lecithin that were absent from the corresponding reaction mixture made with the non-purified soy lecithin, therefore considered to be artefacts of the preparation method. These hydrocarbons in the GC-MS chromatogram included both short and long-chain alkanes. The inventors concluded that other polar lipid preparations that had been purified by TLC might also have yielded these hydrocarbon compounds, and therefore these compounds were excluded from the quantitation of the GC-MS traces for the Y. lipolytica polar lipids as resulting from the sample preparation method. The hydrocarbons not considered in the analysis for this reason were: Hexane, 2,4-dimethyl-; Dodecane, 4,6-dimethyl-; Hexadecane; Heptadecane; Undecane, 3,8-dimethyl-; Triacontane; Hentriacontane; Tetradecane, 5-methyl-; Decane, 3,3,6-trimethyl-; and Hexadecane, 2,6,10,14-tetramethyl-.

### Experiment 5. Maillard Reactions for ARA-PC and 18:1-PC.

Another experiment was carried out to compare the reaction products from Maillard reactions for mixtures including pure ARA-phosphatidylcholine (PC) or 18:0/18:1-PC as a comparison, to identify volatile compounds arising specifically from the ARA-PC. Samples containing 2.5 or 5.0 mg of 18:0/18:1-PC (Catalog No. 850467C, Avanti Polar Lipids) or ARA-PC (Avanti Polar Lipids) were treated in 1 ml volumes as for the previous experiment. HS-SPME-GCMS analysis of the volatiles produced after the heating step showed the presence of numerous compounds which were either increased or decreased in the mixtures having ARA-PC relative to the mixtures having 18:0/18:1-PC, or were present in one mixture and absent or not detected in the other mixture.

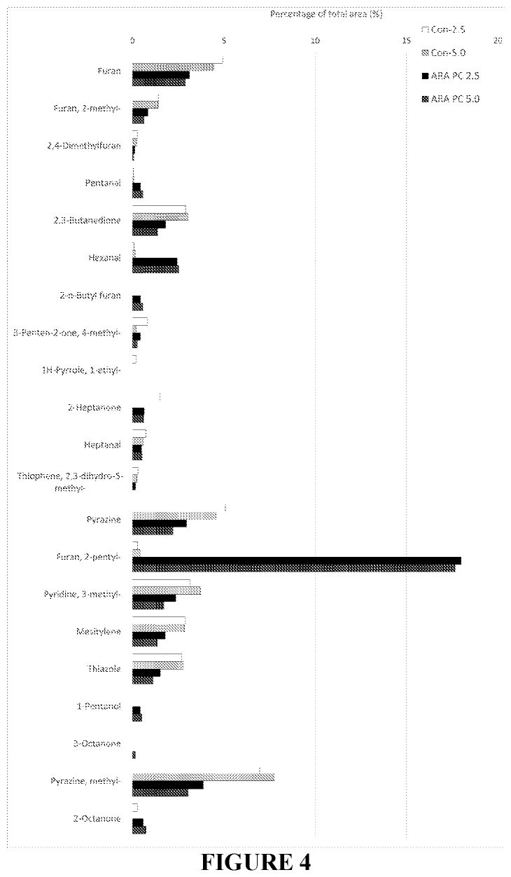

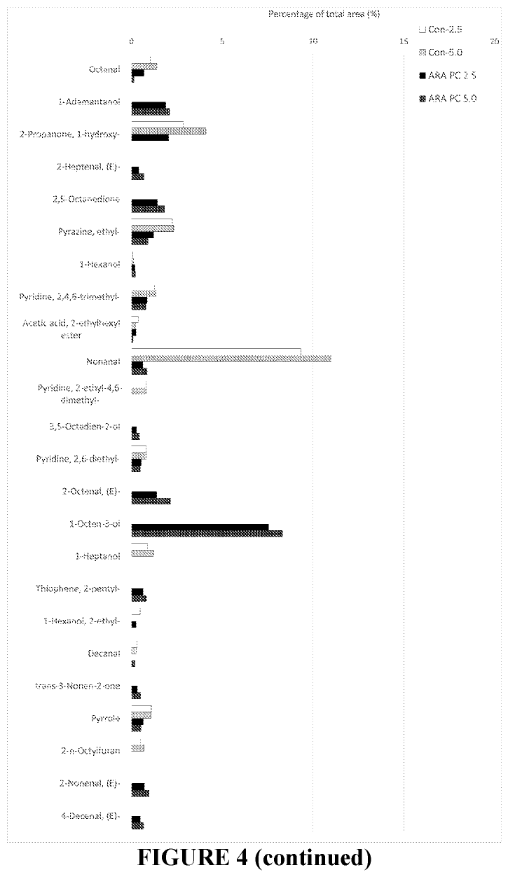

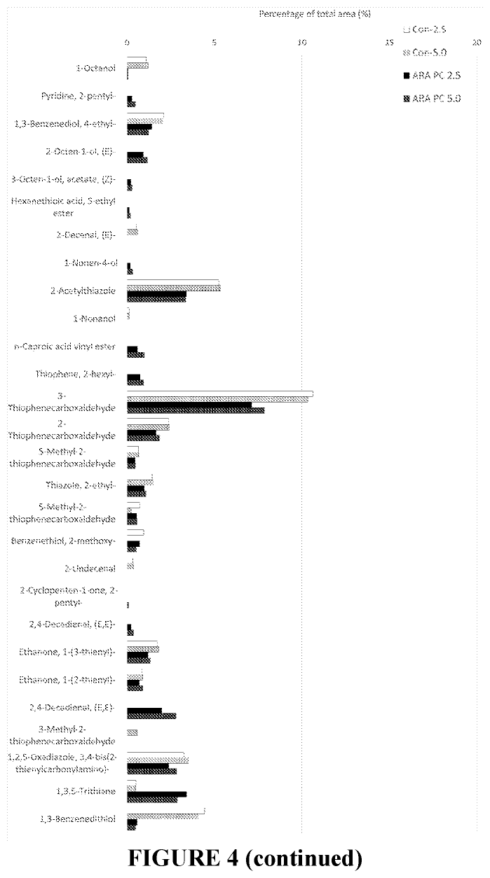

The results are presented in FIG. 4. The results of this experiment demonstrated that alcohols, aldehydes, furans and thiophenes were important volatile compounds found in the reaction mixtures having the ARA-PC lipid. The mixtures derived from ARA-PC showed compounds matching those observed in the earlier experiment, including 1-pentanal, 3-octanone, 2-octen-1-ol, 1-nonanol and 1-octanol. The presence of other compounds was also observed, namely: adamantanol-like compound, hexanal, 2-pentyl furan, 1-octen-3-ol, 2-pentyl thiophene, 1,3,5-thitriane. 2-pentyl thiophene has a characteristic aroma which has been described as chicken, roasted hazelnut, or meaty and bloody. 2-pentyl furan has an aroma described as a fruity, earthy, vegetable aroma.

### Experiment 6.

Larger scale cultures (8 L) of Y. lipolytica strain W29 were grown in the presence of ARA and harvested as described in Example 6 and polar lipid isolated using hexane/ethanol extraction from wet cell pellets as described in Example 8. The yield of extracted lipid from the ethanol phase was 6.422 g, of which 1.974 g (30.7%) was lipid. Of that lipid, 95% was polar lipid and 5% was free fatty acid (FFA); the extracted lipid did not appear to have any TAG. The level of ARA in the total fatty acid content of the polar lipid fraction was only 3.2% (Table 32), so lower than optimal. The inventors nevertheless tested this polar lipid in Maillard reactions, with the conditions as in Experiment 5 except using 15, 30 or 60 mg polar lipid per reaction in 2 ml volumes to increase the amount of ARA-polar lipid. The control polar lipid extract had been prepared from Y. lipolytica cells which had not been fed ω6 fatty acids in the medium. Control reactions were also set up having aliquots of the polar lipid extracts but lacking the ribose and cysteine.

The aromas from the reactions were smelled by three volunteers. The mixtures having the ARA-PL provided mild aromas that were described as “pork like, pork crackling, meaty, fatty” or “broiled chicken, milder aroma” or “like broiled fish” whereas the control mixtures having the polar lipid from Y. lipolytica not fed the ARA was described as being sulphurous or “burnt” in their aroma. The mixtures lacking ribose and cysteine were described as “burnt vegetable”.

The inventors concluded that the polar lipid extract having the lower ARA level at 3.2% could provide meat-like aromas but that levels of 10% ARA or greater were better at providing stronger aromas.

### Experiment 7.

In order to test aromas produced from reactions including polar lipid incorporating either GLA, DGLA, DTA or DPA-ω6, Y. lipolytica was cultured in the presence of one or other of these fatty acids, using the same conditions as described for ARA. One 3 L culture of Y. lipolytica had 0.5 mg/ml (final concentration) of GLA in the medium. Polar lipid produced from cells that had incorporated the GLA had the fatty acid composition shown in Table 32, including 51.8% GLA. This polar lipid preparation appeared to be free of ω3 fatty acids.

Maillard reactions with the GLA-polar lipid and the DGLA-polar lipid preparations are set up as for the ARA-polar lipid described above.

### Experiment 8. Production of Aromas Using Whole Cells Containing 06-Polar Lipids

As described in Example 6 for batches B004 and B005, Y. lipolytica strain W29 cells producing polar lipids including PL were grown in 25 L cultures, either in the presence of ARA (Y1-ARA) in the growth medium or in the absence of ARA (Y1). The fatty acid composition of the polar lipid in the Y. lipolytica cells is shown in Table 32 for Experiments 2 and 3. Notably, ARA was present at 16.4% of the total fatty acid content of the polar lipid, with GLA at 1.4% and DGLA at 1.9%. The harvested cells were then freeze dried and the dried material milled to a powder. The cells were not heat treated or otherwise treated to kill or inactivate the cells. The inventors wished to test the dried yeast cells for the capacity to provide aroma compounds after the cells were heated in the presence of a sugar, for example D-xylose, and an amino acid, for example L-cysteine. A series of reactions were prepared to test the effect of different amounts of the sugar, the amino acid and varying amounts of freeze-dried cells (Table 35). Briefly, L-cysteine powder (Catalog No. 30089, Sigma-Aldrich), D-xylose powder (Catalog No. X1500, Sigma-Aldrich), sodium citrate·2H₂O (Catalog No. W302600, Sigma-Aldrich), and wheat flour were weighed into 10 ml GC headspace analysis vials (Catalog No. 23084, Restek, USA) at the indicated amounts before the addition of freeze-dried yeast cells from cultures with or without added ARA. Water (2 ml or 3 ml) was added to each vial and the lids tightly closed before mixing by brief vortexing. The pH of the mixture for vial number 1 was 6.0, based on the buffering by the sodium citrate. The vials were then incubated in an oven pre-heated to 120° C. for either 60 or 45 min before being cooled on ice. The vials were warmed to room temperature before they were opened, and the contents smelled. The aroma for each vial was recorded (Table 35). It was noticed that the cap to vial 13 had been loosened, so that reaction was repeated as vial 19. The loosened cap on vial 13 was presumed to have allowed escape of some of the volatile compounds during heating. Duplicate samples for vials 18-20 were prepared without the water, kept at ambient temperature for 5 or 7 days before the addition of water and then heated to 120° C. for 60 min. These vials provided the same aroma results as vials 18-20 that had been prepared and heated immediately, then frozen for the week, showing that the mixtures can be stored stably for at least one week at room temperature.

Several observations were noteworthy. Reaction vials 2-4 compared to 5-7 were designed to test the effect of whole yeast cells containing ARA in their lipid, compared to yeast cells that did not contain ARA in their lipid. The difference was clearly noticeable with the production of roast meat aroma from the cells having ARA, compared to vials 2-4 where the aroma was not discernible. When the amount of cysteine was lowered to 0.05 g per vial (e.g. vial 17), it was also difficult to detect the desired roast meat aroma. In contrast, when cysteine was at the highest level (e.g. vial 20), the roast meat aroma was more discernible but overpowered or masked to some extent by a sulphurous aroma. A similar effect was noted with the amount of xylose i.e. a lower xylose concentration resulted in a less noticeable undesirable aroma even in the presence of relatively high cysteine concentration (e.g. vial 13), so this was associated with the cysteine concentration. It was concluded that the levels of the amino acid and sugar could be balanced empirically to provide the optimal aroma, i.e. to achieve adequate production of aroma volatiles from the ω6-PUFA without them being masked by stronger smelling undesirable compounds.

The heating time was also a factor to consider. Vials 14-16 and 17-19 were designed to compare this variable with 45 or 60 min heating. The shorter heating time resulted in a noticeably lighter coloured mixture while the longer cooking time produced considerably browner colour. This darkening effect also appeared to be correlated with cysteine levels with more cysteine generally resulting in a darker reaction as long as adequate sugar was present.

In similar fashion the concentration of whole cells was important for desirable aroma generation as demonstrated by vials 8-10. The lower amount of whole cells used in vial 8 resulted in the production of a faint meaty aroma while increasing the amount (vials 9 and 10) yielded a more readily discernible roast meat aroma. It was therefore important to use adequate amounts of whole cells to provide enough ω6-PUFA incorporated into polar lipids for desirable aromas. Again, this feature can be determined empirically.

This experiment also tested whether the yeast cells in the presence of a more complex, food-like material would change the aroma profile. Most of the tested reactions had simple chemical mixtures but vials 8-10 also contained added whole wheat flour to mimic the effect of the presence of plant proteins, carbohydrates, nucleic acids and other components. The aroma from these vials was noticeably different to corresponding vials without the added flour. The aroma of unpleasant sulphur compounds was moderated while the roast meat aroma was still present, providing a more pleasant aroma. The inventors concluded that the use of whole cells producing ω6 fatty acids in the PL when the cells were incorporated in a food containing plant protein was likely to provide the desirable aroma.

A primary conclusion from this experiment was that the addition of ω6 PUFA-containing phospholipids in whole yeast cells worked as well in producing meat-like aroma as the addition of extracted lipid containing the phospholipids with ω6 PUFA. That is, this experiment indicated that it was not necessary to extract ω6-containing phospholipids from the producing cells in order for them to be effective in Maillard or Amadori reactions to produce desirable aroma volatiles.

This experiment was carried out at a starting pH of 6.0 which was considered to be suitable for promoting Amadori reactions. In further experiments, this parameter is varied to determine the optimal pH.

TABLE 35
Aroma of Maillard reaction products from mixtures comprising Y. lipolytica W29 cells cultured either in the presence
of ARA (Yl-ARA) or in the absence of ARA (Yl). The reactions contained L-cysteine, D-xylose at the indicated amounts.
Vials 1-24 and 28-33 were incubated at 120° C. for 60 min, whereas vials 25-27 were incubated at 120° C. for 45 min.

| | | | | Na | Wheat | | |
|---|---|---|---|---|---|---|---|
| Vial | Water | Cysteine | Xylose | citrate | flour | | |
| No. | (ml) | (g) | (g) | (g) | (g) | Yeast cells (g) | Odour |
| 1 | 2 | 0.5 | 0.2 | 0.3 | 0 | none | strong sulphur, unpleasant |
| 2 | 2 | 0.5 | 0.2 | 0.3 | 0 | 0.10 Yl | garlic, sulphur |
| 3 | 2 | 0.5 | 0.2 | 0.3 | 0 | 0.25 Yl | garlic, sulphur |
| 4 | 2 | 0.5 | 0.2 | 0.3 | 0 | 0.50 Yl | garlic, sulphur |
| 5 | 2 | 0.5 | 0.2 | 0.3 | 0 | 0.10 Yl-ARA | garlic, mild meat aroma |
| 6 | 2 | 0.5 | 0.2 | 0.3 | 0 | 0.25 Yl-ARA | garlic, beefy aroma |
| 7 | 2 | 0.5 | 0.2 | 0.3 | 0 | 0.50 Yl-ARA | garlic, beefy aroma, smoky |
| 8 | 2 | 0.5 | 0.2 | 0.3 | 0.25 | 0.10 Yl-ARA | garlic, mild meat aroma |
| 9 | 2 | 0.5 | 0.2 | 0.3 | 0.25 | 0.25 Yl-ARA | lighter aroma, old beef roast |
| 10 | 2 | 0.5 | 0.2 | 0.3 | 0.25 | 0.50 Yl-ARA | lighter aroma, old beef roast |
| 11 | 2 | 0.05 | 0.13 | 0.1 | 0 | 0.25 Yl-ARA | low sulphur, low garlic, beefy |
| | | | | | | | aroma |
| 12 | 3 | 0.1 | 0.05 | 0.1 | 0 | 0.25 Yl-ARA | low sulphur, low garlic, beefy |
| | | | | | | | aroma, pleasant |
| 13 | 3 | 0.2 | 0.05 | 0.1 | 0 | 0.25 Yl-ARA | garlic, mild meat aroma |
| 14 | 3 | 0.05 | 0.05 | 0.1 | 0 | 0.25 Yl-ARA | very faint meaty |
| | | | | | | | aroma/colour, |
| 15 | 3 | 0.1 | 0.05 | 0.1 | 0 | 0.25 Yl-ARA | lighter aroma/colour, |
| 16 | 3 | 0.15 | 0.05 | 0.1 | 0 | 0.25 Yl-ARA | lighter aroma/colour, |
| 17 | 3 | 0.05 | 0.05 | 0.1 | 0 | 0.25 Yl-ARA | very faint meaty |
| | | | | | | | aroma/colour, |
| 18 | 3 | 0.1 | 0.05 | 0.1 | 0 | 0.25 Yl-ARA | low sulphur, low garlic, beefy |
| | | | | | | | aroma, pleasant |
| 19 | 3 | 0.15 | 0.05 | 0.1 | 0 | 0.25 Yl-ARA | sulphur, garlic, strong meaty |
| | | | | | | | aroma |
| 20 | 3 | 0.3 | 0.18 | 0.18 | 0 | 0.25 Yl-ARA | sulphur, garlic, strong meaty |
| | | | | | | | aroma |

### Example 8. Fractionation of Lipid-Larger Scale

The following experiments were performed to test extraction of phospholipid and separation from TAG at a larger scale, from Y. lipolytica strain W29 cells grown at 2 L or 8 L scale in a fermenter as described in Example 6.

### Experiment 1

The inventors considered that PL could be extracted from microbial cells such as Y. lipolytica and simultaneously be separated from TAG by using a solvent mixture of ethanol: hexane. This method was based on Sun et al. (2019). About 100 g wet weight of Y. lipolytica cells were used, testing whether the TAG and polar lipids could be effectively extracted from the cells and partitioned between a hexane phase and an ethanol-water phase, respectively. In this experiment, 102.12 g wet weight of cells, corresponding to 20.42 g dry weight of cells i.e. about 80% moisture and 20% solids, was mixed overnight with 500 ml of ethanol/hexane (4/6, v/v) by stirring, in a 1 L beaker. This provided a sample/solvent ratio of 1/5 (w/v) based on wet weight, or 1/25 (w/v) based on the dry weight of cells. After the overnight mixing, the mixture had separated into two phases with flocculated cellular material at the interface. The upper, hexane phase was pale yellow in colour, while the lower, ethanol phase was pale green. The mixture was decanted and filtered through a glass fibre filter (1.2 μm, MicroAnalytix Pty Ltd, Catalog No. WH1822-090) using a glass vacuum filtration apparatus to remove the flocculated cellular material, and the residue rinsed with 100 ml of ethanol/hexane (4/6 v/v) solvent, combining the filtrates. The filtrate was separated into two phases in a separatory funnel: an upper, hexane phase containing what was hoped to have most of the TAG and a lower ethanol phase containing what was hoped to have most of the polar lipid including PL. The two phases were collected separately in 1 L round bottom flasks and dried using a rotary evaporator. The dried extracts were weighed, yielding 0.513 g of extracted lipid from the hexane phase (2.51% of the 20.42 g dry cell weight) and 3.69 g of extracted lipid from the ethanol phase (18.06% w/dcw). Both fractions were washed with chloroform to remove any traces of water, adding the chloroform followed by rotary evaporation, repeating this step until addition of chloroform resulted in a clear water-free extract. This resulted in recovery of 0.51 g of lipid from the upper phase and 3.43 g lipid from the ethanol phase.

To transfer the extracted lipid to a tube suitable for transport, the polar lipid extract was dissolved in 26 ml chloroform, transferred to a 50 ml plastic centrifugation tube and the solvent evaporated using a Savant SC250EXP SpeedVac concentrator at 45° C. overnight.

Samples of the extracted lipid fractions were applied to a TLC plate and chromatographed to separate the different lipid classes. As determined by quantitation of FAME, the extract from the upper, hexane phase contained 24.9% lipid, 60% of which was TAG but also containing substantial polar lipid, at 40% of the lipid. The extract from the lower, ethanol phase had 9.5% lipid, of which 95% was polar lipid, and no TAG was detected in that fraction.

To determine the amount of total extractable lipid from Y. lipolytica cells grown under the same conditions, using an established method, and to test whether the ethanol/hexane extraction was efficient enough, lipid was extracted from a similar quantity of cells using a standard procedure based on Bligh and Dyer (1959). Briefly, a frozen, wet cell pellet of 103.08 g having about 20% total solids was placed in a 1 L glass beaker. Chloroform/methanol/water solvent was added comprising 166.7 ml chloroform, 266.3 ml methanol and 53.4 ml water. The frozen pellet was broken into small pieces in the solvent using a spoon. The beaker was covered with aluminium foil and the mixture stirred overnight at room temperature with a magnetic stirrer. The mixture was then vacuum filtered through a glass fibre filter, and the residue of cellular material rinsed with 131 ml chloroform and 103 ml water and filtered again. The total filtrate was transferred to a 1 L separation funnel, shaken gently and allowed to stand for several hours for phase separation to occur. The bottom chloroform layer containing extracted lipid was drained into a 250 ml round bottom flask and the solvent removed by rotary vacuum evaporation. The total lipid extract in the flask was weighed for gravimetric yield determination: the lipid yield of 2.06 g was 9.97% on a dry cell weight basis assuming an 80% moisture content of the wet yeast cell pellet. The total lipid extract was dissolved in 26 ml chloroform and samples converted to FAME and quantitated by GC as described in Example 1.

Samples of the extracted lipid from the Bligh-Dyer method were also applied to a TLC plate and chromatographed to separate the different lipid classes. Standards of known lipid classes were applied to adjacent lanes to identify the lipid spots. This identified the polar lipid and TAG fractions in the Bligh-Dyer extract and allowed their quantification. The Bligh Dyer extract contained 28.3% total lipid, 7% of which was TAG and 93% of which was polar lipid, The extraction using the ethanol/hexane method was nearly as efficient as the Bligh-Dyer method, demonstrating that the ethanol/hexane solvent system was useful.

Samples of each of the fractions were analysed for fatty acid composition by conversion to FAME and GC analysis as described in Example 1, for the extracts from the upper (hexane) and lower (ethanol) phases. The fatty acid composition was also separately determined for the polar lipid and TAG fractions isolated on TLC plates. The data are provided in Table 36. In Experiment 1, the lipid extracted in the lower (ethanol) phase contained predominantly (95%) polar lipid, having a fatty acid composition much lower in the saturated fatty acid C18:0, C22:0 and C24:0 than the TAG extracted in the upper (hexane) phase. In contrast, the level of C16:0 was similar in both the polar lipid and TAG fractions from the same upper phase at between 16-18%.

One conclusion from this experiment was that the lipid extracted from the lower, ethanol phase was almost entirely polar lipid, essentially lacking TAG. However, this method did not extract all of the polar lipid available, leaving considerably polar lipid in the upper hexane phase. The remaining polar lipid can be recovered by degumming procedures. Although the extracted material from the ethanol phase was only 9.5% lipid by weight, the other 90% of the material, presumably ethanol soluble substances such as some proteins and carbohydrates, can be removed to some extent by extraction of the lipids into chloroform, since proteins and carbohydrates are not soluble in chloroform.

### Experiment 2

A second experiment was performed which was the same as Experiment 1 except with a modification to the solvent used. This time, extraction of 99.8 g of wet weight cells initially used 500 ml of ethanol: hexane (6/4 v/v) rather than (4/6 v/v), with subsequent adjustment to (4:6 v: v). This modification was made because, in Experiment 1, the phases tended to separate even at the start of the extraction, possibly due to the amount of moisture in the cell sample. In this second experiment, the cells were mixed with the ethanol/hexane (6/4 v/v) by stirring at room temperature overnight. After that, 250 ml of hexane was added so that the solvent mixture was now ethanol/hexane (4/6 v/v), with mixing for a further 5 min. The rest of the procedure was the same as for Experiment 1. Product was initially recovered from the upper, hexane phase at 1.23 g (6.16% w/dcw) and from the lower, ethanol phase at 4.05 g (20.29% w/dcw). After the chloroform wash and drying of the extracts, the recoveries were 6.16 g and 19.89 g, respectively.

It was concluded from these experiments and the following ones that this solvent extraction method worked quite well as long as there was adequate agitation of the mixture during the overnight extraction. Conditions are varied for optimisation, e.g. solvent volumes, ratios, extraction time and temperature, and starting with wet vs dry cells. It was expected that extraction from dry cells and at a higher temperature would provide a greater yield of total lipid, TAG and polar lipid fractions.

### Experiment 3

Since the batch extractions in Experiments 1 and 2 used large amounts of solvent for the amount of recovered lipid at about 500 ml per 100 g wet weight of cells, it was decided to test extraction using a Soxhlet apparatus (De Castro et al., 2010). This experiment used the same solvent composition as in Experiment 2, starting with ethanol/hexane (6/4, v/v) and then adding hexane to adjust the ratio to (4/6, v/v). A cell sample of 20 g (wet weight) having 4 g dry weight of cells was added to a Soxhlet cup. Extraction used 300 ml ethanol/hexane (6/4, v/v) in the flask, heating the solvent for 3 h using a heating mantle, and cooling of the condenser with running tap water. After the 3 h extraction, the flask was rinsed with 150 ml of hexane, thereby adjusting the solvent ratio to ethanol/hexane (4/6, v/v). The remainder of the procedure was the same as for Experiment 1, with recovery of the lipids from the upper, hexane phase and the lower, ethanol phase. The recovery of lipid from the upper phase was 5.4% (w/dcw) and from the lower phase 17% (w/dcw), so almost the same polar lipid yield was obtained compared to Experiments 1 and 2, and in a shorter time. It was considered that this method had potential for scaling up by using a larger 1 kg or 5 kg Soxhlet apparatus, or even larger pilot scale extractions.

TABLE 36
Fatty acid composition of the extracted lipid in fractions from Y. lipolytica (W29) in Experiments 1 and 2, compared to Bligh Dyer
extraction. The fatty acid composition is shown for the total lipid (Total) and the polar lipid (Polar) and TAG fractions
in each extract, from the top phase (hexane) or bottom phase (ethanol).

| Fraction/ | | | | | C16: | C16: | | | C18: | C18: | | | C18: | | | % | |
|---|---|---|---|---|---|---|---|---|---|---|---|---|---|---|---|---|---|
| experiment | | C14:0 | C15:0 | C16:0 | 1Δ7 | 1Δ9 | C17:1 | C18:0 | 1Δ9 | 1Δ11 | C19:0 | C18:2 | 3ω3 | C22:0 | C24:0 | lipid | g total |
| Upper phase- | Total | 0.2 | 0.3 | 17.1 | 0.5 | 7.5 | 0.8 | 8.4 | 33.0 | 0.5 | 0.1 | 30.3 | 0.0 | 0.2 | 1.6 | 24.9 | |
| Experiment 1 | Polar | 0.4 | 0.6 | 16.6 | 0.5 | 8.7 | 1.2 | 2.0 | 35.7 | 0.5 | 0.1 | 33.3 | 0.1 | 0.0 | 0.3 | 9.0 | 0.062 |
| | TAG | 0.3 | 0.4 | 17.9 | 0.5 | 6.9 | 0.9 | 13.5 | 27.9 | 0.5 | 0.1 | 26.7 | 0.1 | 0.6 | 3.8 | 13.1 | 0.091 |
| Lower phase- | Total | 0.2 | 0.4 | 16.6 | 0.5 | 9.1 | 1.4 | 1.6 | 36.3 | 0.5 | 0.1 | 33.8 | 0.0 | 0.0 | 0.1 | 9.5 | |
| Experiment 1 | Polar | 0.4 | 0.7 | 17.4 | 0.6 | 9.2 | 1.2 | 1.9 | 34.8 | 0.5 | 0.1 | 33.0 | 0.1 | 0.0 | 0.0 | 9.0 | 0.616 |
| Upper phase- | Total | 0.2 | 0.3 | 18.0 | 0.5 | 6.4 | 0.8 | 11.6 | 29.8 | 1.6 | 0.1 | 28.1 | 0.0 | 0.4 | 2.3 | 37.4 | |
| Experiment 2 | Polar | 0.3 | 0.6 | 18.6 | 0.4 | 5.7 | 0.9 | 2.1 | 35.2 | 0.4 | 0.1 | 35.1 | 0.0 | 0.0 | 0.4 | 6.6 | 0.068 |
| | TAG | 0.3 | 0.4 | 18.0 | 0.5 | 6.7 | 0.9 | 14.2 | 28.4 | 0.6 | 0.1 | 25.7 | 0.0 | 0.6 | 3.5 | 26.8 | 0.273 |
| Lower phase- | Total | 0.3 | 0.5 | 16.5 | 0.4 | 8.8 | 1.1 | 2.0 | 35.4 | 1.1 | 0.2 | 33.4 | 0.0 | 0.0 | 0.3 | 12.5 | |
| Experiment 2 | Polar | 0.4 | 0.7 | 16.8 | 0.6 | 9.3 | 1.3 | 1.8 | 35.2 | 0.5 | 0.1 | 33.3 | 0.1 | 0.0 | 0.0 | 11.4 | 0.260 |
| | TAG | 1.0 | 2.6 | 21.7 | 0.4 | 5.4 | 0.6 | 18.0 | 24.7 | 0.4 | 0.1 | 21.1 | 0.0 | 0.6 | 3.3 | 0.3 | 0.007 |
| Bligh-Dyer- | Total | 0.3 | 0.5 | 17.0 | 0.5 | 8.4 | 1.1 | 2.7 | 35.1 | 0.7 | 0.1 | 32.9 | 0.0 | 0.0 | 0.3 | 28.3 | |
| Experiment 1 | Polar | 0.4 | 0.6 | 16.9 | 0.6 | 8.7 | 1.2 | 1.9 | 35.6 | 0.5 | 0.1 | 33.3 | 0.0 | 0.0 | 0.2 | 25.1 | 0.681 |
| | TAG | 0.4 | 0.6 | 18.8 | 0.5 | 7.1 | 0.9 | 13.0 | 28.0 | 0.5 | 0.1 | 27.0 | 0.0 | 0.5 | 2.6 | 2.0 | 0.054 |

### Experiment 4

A larger batch extraction was performed on 900 g (wet weight) of cells using the solvent system as in Experiment 2, with an initial ratio of ethanol/hexane at 6/4 (v/v), then adjusted to ethanol/hexane at 4/6 (v/v). The extraction was carried out in three 2 L flasks. The recoveries of the lipid from the upper phase was 10.89 g, mostly TAG, and from the lower phase 32.45 g, mostly PL. This represented a 18% yield (w/DCW) of the extracted PL. The PL oil fraction was quite viscous but was able to be transferred from the evaporator flask by warming it to 50° C.

### Experiment 5

An experiment was carried out to compare the efficiency of lipid extraction from wet Y. lipolytica cells relative to freeze-dried cells, using ethanol/hexane at 6/4 (v/v) at a warmer temperature of 50° C. compared to previous experiments at room temperature. The extractions were done for 3 h or with a Soxhlet apparatus using the same solvent for 3 h. After the extraction was completed, the ratio of ethanol/hexane was adjusted to 4/6 (v/v) by addition of more hexane. The Y. lipolytica strain W29 cells had been grown in the presence of ARA to incorporate the ω6 fatty acid into polar lipids. The mixture of the solvent with the dried cells resulted in a single phase, whereas the corresponding mixture from the wet cells resulted in two phases due to the water content. Some water was therefore added to half of the former mixture which, after mixing, resulted in the separation of two phases. The mixtures were filtered to remove cell debris and lipid was recovered from each of the phases. The results are presented in Table 37. For the dry cells, lipid was recovered from the single-phase extract and separately from the two phases after the addition of some water.

TABLE 37
Yield of recovered lipid extracts from Y. lipolytica cells.

| | | Solvent | | Recovered | Total lipid |
|---|---|---|---|---|---|
| Extraction | Sample | mixture | Upper or | lipid | recovered |
| method | mass (g) | volume (ml) | lower phase | (% of dcw) | (% of dcw) |
| Wet cells/50° C. | 250.1 | 1250 | Upper | 3.6 | 14.4 |
| Batch extraction | | | Lower | 10.8 | |
| Wet cells/Soxhlet | 27.2 | 300 in reflux | Upper | 3.0 | 12.7 |
| extraction | | flask | Lower | 9.7 | |
| Dried cells/50° C. | 50.1 | 1250 | Single phase* | 9.9 | 9.2 |
| Batch extraction | | | Upper | 4.4 | |
| | | | Lower | 4.1 | |
| Dried cells/Soxhlet | 15.0 | 300 in reflux | Single phase* | 6.9 | 6.7 |
| extraction | | flask | Upper | 3.5 | |
| | | | Lower | 3.1 | |

*Single phase refers to half of the volume (800 ml) from the dried cell extractions since there was no phase separation prior to adding water.

Blending with Other Lipids

As an alternative for transfer of the polar lipid fraction and to provide blends of the polar lipid with other lipids having functional properties, the inventors dissolved the extracted polar lipid in cocoa butter. Since cocoa butter is a solid at room temperature, 6.5 g was melted at 60° C. and added to 14.42 g of the dried extracted polar lipid. The mixture solidified upon cooling below 34-38° C. although some liquid separated out. A less viscous oil such as canola oil could be used to dissolve the polar lipid. Alternatively, the total lipid including the TAG fraction could be extracted from the microbial cells to provide a more free-flowing oil, or the purified polar lipid fraction could be formulated as a powder for easier transport and incorporation into foods.

Fractionation of Polar and Non-Polar Lipids by Precipitation from Acetone.

An experiment was carried out to test whether polar lipid containing ω6 fatty acids and a non-polar lipid could be separated, or at least enriched for the ω6 content, using precipitation from an organic solvent at defined temperatures. To do this, 97 mg of cocoa butter (Societe Africaine De Cacao) and about 2 mg of polar lipid extracted from Y. lipolytica cells that had been cultured in the presence of ARA, having about 16.4% ARA in the total fatty acid content, were mixed in a 15 ml tube at 50° C. The lipid was dissolved in 5 ml of acetone by ultrasonication of the mixture in a water bath at 40° C. for 5 min and then mixing at 37° C. for 15 min. Cocoa butter was used because it is rich in saturated fatty acids. The lipid mixture in acetone was incubated at 20° C. with mixing for 24 h. No precipitate was clearly observed at this temperature. However, the mixture was centrifuged at 4,600 g for 15 min and the supernatant was transferred to a new tube, which was incubated at 15° C. for 24 h. The first tube was stored at −20° C. for lipid analysis of a small pellet that was observed. After centrifuging the mixture as before, the 15° C. supernatant was transferred to a new tube and incubated at 12.5° C. for 24 h, after which considerable precipitation was observed. The mixture was again centrifuged and the supernatant transferred to a new tube. The pellet was washed with 2 ml of cold (12.5° C.) acetone by gentle mixing and the supernatant was combined with the earlier supernatant, which was incubated at 10° C. for 3 days. After centrifugation, the supernatant was again transferred to a new tube, the pellet was washed with 2 ml cold acetone and the supernatant combined with the earlier supernatant, which was incubated at 4° C. for 24 h. After centrifugation, the supernatant was again collected in a new tube, the pellet was washed with 2 ml cold acetone and the supernatant was combined with the 4° C. supernatant. The acetone was evaporated from all of the pellets and supernatants under a flow of nitrogen at room temperature and the dried, recovered lipids were dissolved in chloroform. The TAG and polar lipids classes of the precipitates and supernatant fractions were separated by TLC chromatography using hexane/diethylether/acetic acid (70/30/1) and quantitated and analysed for fatty acid composition by GC of FAME.

The data are shown in Table 38. TAG and polar lipid were precipitated at all the temperatures tested, namely 20° C., 15° C., 12.5° C., 10° C. and 4° C. The greatest amount of TAG was precipitated at 12.5° C. (41%), followed by 21.9% at 4° C., while 30.7% of the polar lipid precipitated at 20° C. Significantly, most (60.9%) of the polar lipid remained in the supernatant at 4° C., with an enrichment of the level of ARA from 16.4% to 24.8% of the total fatty acid content of the polar lipid. The supernatant at 4° C. also contained 27.4% of the total TAG. Polar lipid precipitated at 20° C. and 15° C. were enriched in C18:1 and C18:2, while those from 12.5° C., 10° C. and 4° C. were enriched in C16:0 and C18:0 and contained lower proportions of ARA. On the other hand, the TAG that precipitated at the higher temperature contained higher levels of C18:0 whereas those from lower temperatures were richer in C16:0, C18:1 and C18:2. Although this method did not result in a pure fraction of polar lipid, it can be used to increase the ω6 fatty acid content in polar lipid and to increase the polar lipid/non-polar lipid (TAG) ratio in an extracted lipid sample. Further optimization work can be done by seeding the mixture with TAG crystals to enhance TAG precipitation and application of lower temperatures than 4° C.

TABLE 38
Fatty acid composition of lipid precipitates (PPT) and supernatants (SUP) after
acetone fractionation.

| Polar lipid | | | | | | | | | | C18: | C18:2 |
|---|---|---|---|---|---|---|---|---|---|---|---|
| fractions | C12:0 | C12:1 | C14:0 | C15:0 | C16:0 | C16:1 | C17:1 | C18:0 | C18:1 | 1Δ11 | (LA) |
| PPT 20° C. | 0.0 | 0.2 | 0.1 | 1.2 | 17.4 | 10.2 | 1.3 | 0.7 | 42.0 | 1.0 | 13.4 |
| PPT 15° C. | 0.4 | 4.9 | 0.4 | 1.2 | 18.3 | 8.6 | 1.0 | 7.2 | 33.6 | 0.0 | 9.8 |
| PPT 12.5° C. | 0.0 | 10.7 | 0.5 | 0.0 | 24.1 | 4.9 | 0.7 | 16.5 | 27.1 | 0.8 | 6.6 |
| PPT 10° C. | 0.2 | 7.1 | 0.5 | 0.0 | 25.1 | 5.5 | 0.8 | 15.5 | 29.0 | 0.0 | 8.1 |
| PPT 4° C. | 0.2 | 2.8 | 0.3 | 1.6 | 25.1 | 7.1 | 0.9 | 7.3 | 34.0 | 0.6 | 8.4 |
| SUP 4° C. | 0.1 | 0.6 | 0.2 | 0.9 | 11.8 | 11.0 | 1.4 | 2.9 | 35.5 | 0.7 | 7.9 |
| TAG | | | | | | | | | | | |
| fractions | | | | | | | | | | | |
| PPT 20° C. | 0.1 | 15.1 | 0.2 | 0.0 | 22.8 | 0.4 | 0.0 | 30.8 | 26.8 | 0.5 | 2.2 |
| PPT 15° C. | 0.0 | 1.8 | 0.0 | 0.0 | 17.0 | 0.1 | 0.0 | 51.6 | 26.6 | 0.3 | 0.8 |
| PPT 12.5° C. | 0.0 | 1.2 | 0.0 | 0.0 | 19.7 | 0.1 | 0.0 | 44.4 | 32.6 | 0.0 | 0.6 |
| PPT 10° C. | 0.0 | 2.0 | 0.0 | 0.0 | 25.7 | 0.1 | 0.0 | 38.4 | 31.9 | 0.0 | 0.8 |
| PPT 4° C. | 0.0 | 0.8 | 0.1 | 0.0 | 34.3 | 0.1 | 0.0 | 30.1 | 31.8 | 0.3 | 1.6 |
| SUP 4° C. | 0.0 | 1.2 | 0.2 | 0.1 | 34.7 | 0.8 | 0.0 | 20.0 | 32.0 | 1.2 | 8.1 |

Fatty acid composition of lipid precipitates (PPT) and supernatants (SUP) after
acetone fractionation.

| | C18: | C18: | | | C20: | | | | | | |
|---|---|---|---|---|---|---|---|---|---|---|---|
| Polar lipid | 3ω6 | 3Δ3 | | | 3ω6 | | | | | | % of |
| fractions | GLA | ALA | C20:0 | C20:1 | DGLA | ARA | C22:0 | C22:1 | C24:0 | mg | total |
| PPT 20° C. | 0.8 | 0.0 | 0.0 | 0.1 | 0.2 | 11.4 | 0.0 | 0.0 | 0.1 | 0.27 | 30.7 |
| PPT 15° C. | 1.2 | 0.0 | 0.0 | 0.0 | 0.0 | 12.7 | 0.0 | 0.7 | 0.0 | 0.02 | 1.8 |
| PPT 12.5° C. | 0.8 | 0.0 | 0.0 | 0.7 | 0.0 | 6.6 | 0.0 | 0.0 | 0.0 | 0.02 | 1.8 |
| PPT 10° C. | 0.7 | 0.0 | 0.0 | 0.5 | 0.0 | 5.8 | 0.0 | 1.1 | 0.0 | 0.00 | 0.4 |
| PPT 4° C. | 0.7 | 0.0 | 0.3 | 0.3 | 0.2 | 9.6 | 0.2 | 0.4 | 0.0 | 0.04 | 4.4 |
| SUP 4° C. | 1.4 | 0.2 | 0.1 | 0.1 | 0.3 | 24.8 | 0.1 | 0.0 | 0.1 | 0.54 | 60.9 |
| TAG | | | | | | | | | | | |
| fractions | | | | | | | | | | | |
| PPT 20° C. | 0.0 | 0.1 | 0.8 | 0.0 | 0.0 | 0.0 | 0.1 | 0.0 | 0.1 | 0.26 | 0.4 |
| PPT 15° C. | 0.0 | 0.0 | 1.4 | 0.0 | 0.0 | 0.0 | 0.2 | 0.0 | 0.1 | 4.4 | 6.4 |
| PPT 12.5° C. | 0.0 | 0.0 | 1.2 | 0.0 | 0.0 | 0.0 | 0.1 | 0.0 | 0.1 | 28.4 | 41.0 |
| PPT 10° C. | 0.1 | 0.0 | 0.9 | 0.0 | 0.0 | 0.0 | 0.1 | 0.0 | 0.0 | 2.0 | 2.9 |
| PPT 4° C. | 0.0 | 0.1 | 0.6 | 0.0 | 0.0 | 0.0 | 0.1 | 0.0 | 0.1 | 15.2 | 21.9 |
| SUP 4° C. | 0.0 | 0.5 | 0.7 | 0.1 | 0.0 | 0.0 | 0.1 | 0.0 | 0.1 | 19.0 | 27.4 |

### Example 9. Modification of Microbes to Reduce Polyunsaturated Fatty Acids

Many yeasts produce polyunsaturated fatty acids (PUFA) including linoleic acid (LA, C18:2Δ9,12) and α-linolenic acid (ALA, C18:3Δ9,12,15) which are incorporated into their oil, including in TAG, and in their membrane lipids such as phospholipids. Production of ω6 fatty acids including LA requires the activity of a Δ12 desaturase which is encoded by a FAD2 gene, whereas incorporation of the third double bond to produce ALA from LA additionally requires a Δ15 desaturase. When cultured in a rich medium lacking added fatty acids, the wild-type Y. lipolytica strain W29 produced the ω6 fatty acid LA (Example 4, Table 10). In some samples, strain W29 also produced trace amounts of the ω6 fatty acid C20:2Δ11,14 which was a two-carbon extension product of LA. Strain W29 appeared to lack a A15 desaturase since ALA was absent from the TAG and phospholipid. A FAD2 gene was cloned from Y. lipolytica by Yadav and Zhang (WO2004/104167) and Tezaki et al. (2017) and shown to encode the Δ12 desaturase. They also generated a deletion mutant (fad2) which did not produce LA. That mutant was compromised in its growth at 12° C. in the absence of added LA in the growth medium.

Y. lipolytica Δ12 desaturase (SEQ ID NO:2) is a protein of 419 amino acid residues. The protein contains three histidine motifs, typical for fatty acid desaturases, at amino acid positions 121-125, 157-161 and 343-347. These histidine motifs are highly conserved among all FAD2 homologs. When the Y. lipolytica Δ12 desaturase was compared to other microbial desaturases, the protein was related but phylogenetically distinct from the Δ12- and A15-desaturases of the ascomycetous yeasts L. kluyveri (Accession No. Q765N3), K. pastoris (Q5BU99), K. lactis (Q6CKY7), C. albicans (Q59WT3), C. parapsilosis (C3W956) and O. polymorpha (E5DCJ6) (Tezaki et al., 2017). Multiple sequence alignment of FAD2 homologs revealed that the fungal homologs exhibited at least 46% sequence homology within the fatty acid desaturase domain (PF00487), which spanned the amino acid region from 102-375.

Genetic Constructs for Introducing a FAD2 Gene Deletion into Y. lipolytica

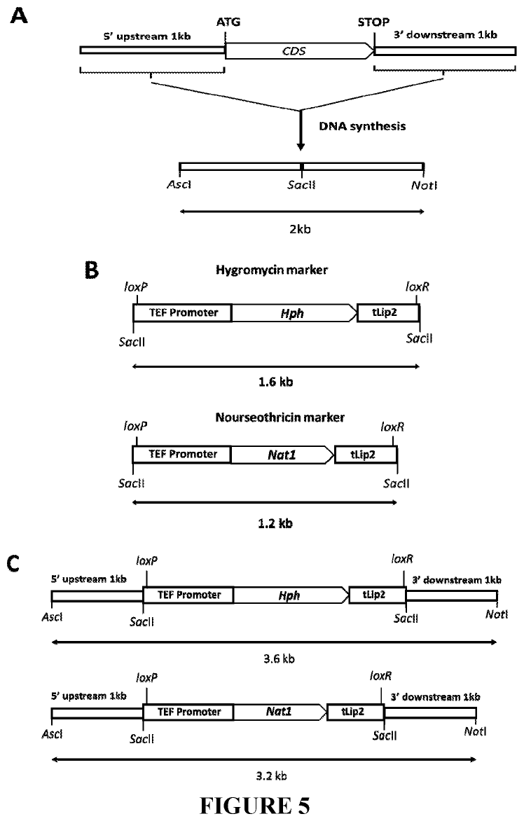

The present inventors wished to test the ability of exogenous Δ12 desaturases to convert oleic acid to LA for the production of ω6 fatty acids, using a fad2 null mutant of Y. lipolytica to do this, and to compare the ability of the fad2 mutant to incorporate ω6 fatty acids into polar lipids compared to the corresponding wild-type strain. To delete the protein coding sequence of the FAD2 gene from the Y. lipolytica genome and thereby inactivate the gene completely, providing a null mutation, the general strategy of Fickers et al. (2003) was used, with modifications for use of different restriction enzyme sites, as follows. A schematic representation of the strategy is shown in FIG. 5. The strategy involved construction of a genetic cassette which had the protein coding region of the gene of interest replaced with a selectable marker gene, flanked by 5′ upstream and 3′ downstream sequences which provided for integration of the genetic cassette by recombination into the endogenous gene, so deleting the protein coding region. The genetic constructs used a selectable marker gene which provided resistance to an antibiotic, either hygromycin or nourseothricin, providing selection alternatives as appropriate for the context. The 5′ upstream and 3′ downstream sequences of 1,000 base pairs each were homologous to the target gene to allow for recombination in each region.

The nucleotide sequence of the FAD2 gene of Y. lipolytica strain W29 and its upstream and downstream sequences were extracted from the KEGG Yarrowia database (www.genome.jp/kegg-bin/show_organism?org-yli), as gene YALI0B10153p, Accession No. XP_500707, using the known amino acid sequence as a query. SEQ ID NO:62 herein provides the nucleotide sequence of the FAD2 gene including 1,000 nucleotides upstream of the protein coding sequence, presumably including the FAD2 promoter, followed by the protein coding sequence and 1,000 nucleotides downstream of the protein coding sequence.

A DNA fragment corresponding to the 5′ upstream sequence of 1,000 base pairs joined through a SacII restriction enzyme site to the 3′ downstream sequence of 1,000 base pairs was synthesised by GeneArt (Thermofisher, USA). The DNA fragment had flanking AscI and NotI restriction sites which were used to insert the fragment into a pMK vector, forming the construct pAT042. The nucleotide sequence of the cloned insert was confirmed. By joining the 5′ upstream sequence to the 3′ downstream sequence without an intervening FAD2 protein coding sequence, this arrangement effectively deleted the FAD2 protein coding sequence of 1,260 base pairs (Δfad2).

### Selectable Marker Genes

Genetic cassettes for providing resistance to the antibiotics hygromycin (Hyg) or nourseothricin (Nat1) as described by Larroude et al. (2018) were obtained from Addgene (Watertown, MA. USA), identified as constructs GGE367 and GGE368, respectively. Each of the genes was under the control of a promoter from a translation elongation factor-la (pTEF) gene from Y. lipolytica (Muller et al., 1998) which is a strong, constitutive promoter in Y. lipolytica, and a Y. lipolytica strain U6 lipase 2 gene polyadenylation region/transcription terminator (tLip2; Darvishi et al., 2011; Accession No. HM486900). The DNA fragments including the Hyg and Natl transcriptional units from GGE367 and GGE368 were modified by PCR to add a SacII restriction site at each end by using oligonucleotide primers at003 and at004 (Table 39). The modified DNA fragments were ligated into the vector pCR Zero Blunt TOPO (Thermofisher USA; Cat. No. 450245) and the nucleotide sequences of the cloned fragments confirmed. The resultant genetic constructs containing the Hyg and Natl sequences were designated pAT121 and pAT122, respectively (Table 40). In a pair of analogous modifications, the DNA fragments including the Hyg and Natl transcriptional units were modified by using primers at229 and at230 (Table 40) to add flanking AsiSI sites, generating pAT123 and pAT124.

In the process used to add the flanking SacII restriction sites, the design of the primers provided for the retention of the loxR site at the 5′ end of the TEF promoter and the loxP site at the 3′ end of the Lip2 terminator (FIG. 5), thus flanking the Hyg and Natl resistance gene cassettes. These recombinational sites were retained so that the resistance genes, after integration into the microbial genome, could subsequently be excised by Cre/lox recombination. This design allowed for re-use of the same selectable marker gene in multiple rounds of gene deletions, as described further below.

A sample of DNA of pAT121 was digested with SacII, electrophoresed on an agarose gel, and the fragment with the hygromycin resistance gene purified from the gel using a gel extraction kit (Qiagen, USA, Cat. No. 28704). The DNA fragment was then ligated to pAT042 which had been digested with SacII and treated with calf intestinal alkaline phosphatase (New England Biolabs, USA). The ligation mix was introduced into E. coli DH5α competent cells by a standard transformation method. Kanamycin resistant colonies were selected. DNA was prepared from five colonies and screened by digestion with the restriction enzymes Xmal, AscI and NotI and agarose gel electrophoresis to identify and confirm that the correct insertion of the Hyg resistance cassette had occurred into the SacII site between the 5′ upstream and 3′ downstream FAD2 sequences. The resultant constructs having the Hyg antibiotic resistance gene sequences flanked by the 5′ upstream and 3′ downstream sequences from FAD2 was retained and designated pAT259. An analogous construction using the nourseothricin resistance gene cassette (Nat1) resulted in the generation of a genetic construct designated as pAT260 (Table 40 and FIG. 5).

Introduction of a FAD2 Deletion Construct into Y. lipolytica

To introduce the genetic construct in pAT259 containing the hygromycin resistance gene into Y. lipolytica and identify genetically modified cells from the transformation, the following protocol was followed. Cells of Y. lipolytica strain W29 to be transformed were streaked onto a YPD-agar plate and incubated at 28° C. for 16 h. A loopful of the freshly grown cells was scraped from the agar surface. The cells were washed in 1 mL of TE buffer (10 mM Tris-HCl, 1 mM EDTA, pH 8.0) and pelleted by centrifugation at 15,800 g for 1 min at room temperature. The cells were resuspended in 600 μL of 0.1 M lithium acetate (LiAc) solution and incubated at 28° C. for 1 h to generate competent cells. The cell suspension was then centrifuged at 400 g for 2 min at room temperature and the cells gently resuspended in 60 μL of 0.1 M LiAc solution. 40 μL of the competent cells were transferred to a 2 mL tube and mixed gently with 3 to 10 μL (˜500 to 1,000 ng DNA) of AscI/NotI linearized DNA vector and 3 μL of carrier DNA (5 mg/mL). The mixtures were incubated at 28° C. for 15 min. 350 μL of PEG 4000 solution was added to each transformation and mixed gently. The mixtures were incubated at 28° C. for 1 h, followed by a heat shock at 39° C. for 10 min. 600 μL of LiAc 0.1M solution was added and mixed gently.

Each transformation mix was cultured in 5 mL of non-selective medium (YPD) for 24 h to provide for recovery of transformants. The cells were then diluted, plated onto selective YPD medium containing hygromycin (250 μg/mL), and the plates incubated at 28° C. for 2 days to obtain 50-100 colonies per plate. When nourseothricin was used as the selective agent in combination with introduction of the Natl gene in an analogous construction, the antibiotic was used at a concentration of 400 μg/mL.

Hygromycin resistant colonies from the Y. lipolytica transformation were screened by PCR for the FAD2 gene insertion using oligonucleotide primers at239 and at240 and colonies that were positive for the gene deletion/insertion of Hyg were selected. To test the phenotype and confirm the FAD2 deletion mutations, four hygromycin resistant colonies were grown in YPD medium at 28° C. and the fatty acid composition of the total lipid extracted from cells determined by GC quantitation of FAME. The results (Table 41) showed that the lipids from all four transformants lacked LA, confirming that the FAD2 gene was inactivated in each isolate with a concomitant increase in the level of oleic acid to about 76% of the total fatty acid content. Strains which were wild-type for FAD2 and included as controls were grown under the same conditions. They had lipid with about 18% LA and 56% oleic acid. The observed fatty acid composition of the lipid in the fad2 mutants was similar to that reported in WO2004/104167, at 74% oleic acid and no detectable LA.

TABLE 39
Primer sequences
to create deletional mutations in endogenous genes.

| | | | SEQ |
|---|---|---|---|
| | | | ID |
| Primer | Gene/KO | Sequence | NO |
| at003 | SacII-Hyg/Nos_F | GGGGGCGCCTAGGGATAACAGGGTAATGATAAC | 20 |
| at004 | SacII-Hyg/Nos_R | CCCGGCGCCATTACCCTGTTATCCCTACTCGC | 21 |
| at213 | LRO_DelCas5F | GGGGGCGCGCCGAGTCGACTTTCCGATACAGAAG | 22 |
| at214 | LRO_DelCas5R | CCCGCGATCGCTGATGGTTGTGATCAACCGGAAA | 23 |
| at215 | LRO_DelCas3F | GGGGCGATCGCAGAGTCCGTTTTGTAGAGTAATATG | 24 |
| at216 | LRO_DelCas3R | CCCGCGGCCGCACGGACCTCGCAAGTTGCAAAAAG | 25 |
| at217 | DGA1_DelCas5F | GGGGGCGCGCCATGCTGCGGGCGGATCCTGG | 26 |
| at218 | DGA1_DelCas5R | CCCGCGATCGCAGCTTTTGTTTTGTGTGACTTGTC | 27 |
| at219 | DGA1_DelCas3F | GGGGCGATCGCGGAAAACTGCCTGGGTTAGGC | 28 |
| at220 | DGA1_DelCas3R | CCCGCGGCCGCTCTGATGGCCTGGAGCGAGTT | 29 |
| at221 | DGA2_DelCas5F | GGGGGCGCGCCTGGGAGTGTATTTGGAAAATGACT | 30 |
| at222 | DGA2_DelCas5R | CCCGCGATCGCTTTGCGGGCGGTACGGGTAC | 31 |
| at223 | DGA2_DelCas3F | GGGGCGATCGCCATAACACTCATCAGTAGCCTTTAC | 32 |
| at224 | DGA2_DelCas3R | CCCGCGGCCGCTCTTGTAATTCCATAGATAATATATACG | 33 |
| at225 | ARE1_DelCas5F | GGGGGCGCGCCGCCGACAATCTCTCTTCCTCATT | 34 |
| at226 | ARE1_DelCas5R | CCCGCGATCGCTGTGTGTGCGGAGAGTGTCTTG | 35 |
| at227 | ARE1_DelCas3F | GGGGCGATCGCGCACAGTCGCTTCACCACTTG | 36 |
| at228 | ARE1_DelCas3R | CCCGCGGCCGCTAAAACAATGACAAATACAACTCTAG | 37 |
| at229 | AsiSI-Hyg/Nos_F | GGGGCGATCGCTAGGGATAACAGGGTAATGATAAC | 38 |
| at230 | AsiSI-Hyg/Nos_R | CCCGCGATCGCATTACCCTGTTATCCCTACTCGC | 39 |
| at239 | Fad2-gene_F | GTGTCCGAGCCCGTCTACG | 40 |
| at240 | Fad2-gene_R | ACATGGTGATACCAATACCGAC | 41 |
| at241 | AREIKOScreen_F1 | GATGCAAATGACGCACGGCC | 42 |
| at242 | ARE1KOScreen_F2 | CCAAACGACACTCGATAGTG | 43 |
| at243 | ARE1KOScreen_R1 | TGCTTTGCGTGGGAGAGCTC | 44 |
| at244 | ARE1KOScreen_R2 | CAGTGGTTGACATTCTATAGGCAC | 45 |
| at245 | DGA1KOScreen_F1 | CAGGCAACAGACAAGTCACAC | 46 |
| at246 | DGA1KOScreen_F2 | CATCTGGCTCCAACGGCCAC | 47 |
| at247 | DGA1KOScreen_R1 | CAGCTGTGGGTCGTTTTCGC | 48 |
| at248 | DGA1KOScreen_R2 | GCTATTTGCCTAACCCAGGC | 49 |
| at249 | DGA2KOScreen_F1 | CATAGCTACACCCAAAGTCGC | 50 |
| at250 | DGA2KOScreen_F2 | CGCTCAGCGGACTGACCATG | 51 |
| at251 | DGA2KOScreen_R1 | TCTTGGGGTACACGGGCTGG | 52 |
| at252 | DGA2KOScreen_R2 | CTGTAAAGGCTACTGATGAGTG | 53 |
| at257 | LRO1KOScreen_F1 | CATGACTAATCATTCACGCGAC | 54 |
| at258 | LRO1KOScreen_F2 | CGTGGTCGGTGTGATAGCCG | 55 |
| at259 | LRO1KOScreen_R1 | GTTCGCAGCAGATCGGCTCG | 56 |
| at260 | LRO1KOScreen_R2 | CGAACGCCCTTCTCCATCAGTG | 57 |
| at270 | URA3KOScrnF1 | CGTTGGAGGCTGTGGGTCTG | 58 |
| at271 | URA3KOScrnF2 | AGTGCTCAAGCTCGTGGCAG | 59 |
| at272 | URA3KOScrnR1 | GATCTCGGTTCTGGCCGTAC | 60 |
| at273 | URA3KOScrnR2 | GCCTCCAGGAAGTCCATGGG | 61 |

TABLE 40
Genetic constructs made for gene deletions.

| Construct | Description |
|---|---|
| pAT042 | YlFAD2 5′ upstream and 3′ downstream joined |
| pAT069 | YlURA3 5′ upstream and 3′ downstream joined, in pMK-T |
| pAT070 | YlURA3 5′ upstream and 3′ downstream joined, in pMK-RQ |
| pAT121 | Hyg resistance gene with added SacII sites (from GGE367) |
| pAT122 | Nat1 resistance gene with added SacII sites (from GGE368) |
| pAT123 | Hyg resistance gene with added AsiSI sites (from GGE367) |
| pAT124 | Nat1 resistance gene with added AsiSI sites (from GGE368) |
| pAT251 | ARE1 5′ upstream and 3′ downstream joined, with AsiSI site |
| pAT253 | DGA1 5′ upstream and 3′ downstream joined, with AsiSI site |
| pAT254 | DGA2 5′ upstream and 3′ downstream joined, with AsiSI site |
| pAT256 | LRO1 5′ upstream and 3′ downstream joined, with AsiSI site |
| pAT257 | URA3-Hyg-Del Cassette (pAT070 + pAT121) |
| pAT258 | URA3-Nos-Del Cassette (pAT070 + pAT122) |
| pAT259 | FAD2-Hyg-Del Cassette (pAT042 + pAT121) |
| pAT260 | FAD2-Nat1-Del Cassette (pAT042 + pAT122) |
| pAT261 | ARE1 Hyg Del Cassette (pAT251 + pAT257) |
| pAT262 | ARE1 Nat1 Del Cassette (pAT251 + pAT258) |
| pAT265 | DGA1 Hyg Del Cassette (pAT253 + pAT257) |
| pAT266 | DGA1 Nos Del Cassette (pAT253 + pAT258) |
| pAT267 | DGA2 Hyg Del Cassette (pAT254 + pAT257) |
| pAT268 | DGA2 Nat1 Del Cassette (pAT254 + pAT258) |
| pAT271 | LRO1 Hyg Del Cassette (pAT256 + pAT257) |
| pAT272 | LRO1 Nat1 Del Cassette (pAT256 + pAT258) |

TABLE 41
Fatty acid composition of lipid from fad2 deletion
mutants of Y. lipolytica, showing only the
main 5 fatty acids present in wild-type strains.

| | C16:0 | C16:1 | C18:0 | C18:1Δ9 | C18:2Δ9, 12 |
|---|---|---|---|---|---|
| FAD2-1 | 10.1 | 10.2 | 3.3 | 76.4 | 0.0 |
| FAD2-5 | 10.2 | 9.7 | 3.7 | 76.4 | 0.0 |
| FAD2-6 | 10.3 | 9.5 | 3.8 | 76.3 | 0.0 |
| FAD2-9 | 10.4 | 9.4 | 3.5 | 76.8 | 0.0 |
| Average for | 10.2 | 9.7 | 3.6 | 76.5 | 0.0 |
| fad2 deletion | | | | | |
| KO27-1 | 12.4 | 9.1 | 3.9 | 55.8 | 18.8 |
| KO27-2 | 12.4 | 8.9 | 4.2 | 57.1 | 17.3 |
| KO27-3 | 12.4 | 9.2 | 3.7 | 55.5 | 19.2 |
| Average | 12.4 | 9.1 | 3.9 | 56.1 | 18.4 |
| wild-type FAD2 | | | | | |

Production of Phospholipids from the Fad2 Mutant of Y. lipolytica

The inventors tested the ability of the fad2 mutant to incorporate ω6 fatty acids into polar lipid and compared the mutant with the corresponding wild-type strain (Example 5 above). The mutant was grown in YPD medium for up to 3 days in the absence of ARA or in the presence of 0.1 mg/ml or 0.5 mg/ml of pure ARA (Nu-Chek Prep Inc.), final concentration. The cultures were sampled at 24, 48 and 72 h and the fatty acid composition and amount of the polar lipid and TAG fractions were determined.

The results are presented in Table 42. In the absence of ARA added to the growth medium, the fatty acid composition of the polar lipid and TAG fractions in the fad2 mutant either contained only a trace amount of LA, just detectable, or LA was absent. All other ω6 fatty acids were not detected. When ARA was added to the medium, low amounts of LA, GLA and DGLA were observed in both the polar lipid and the TAG fractions, presumably due to some catabolism of the ARA added to the medium to the shorter fatty acids or to slight impurity in the ARA. Most notably, ARA was incorporated into both the polar lipid and TAG fractions in a dose-dependent manner. For example, the ARA level was 17.0% of the total fatty acid content of the polar lipid when 0.5 mg/ml ARA was used, compared to 5.5% when 0.1 mg/ml was used. The inventors considered that higher levels of ARA incorporation would be achieved with increased ARA concentrations in the growth medium. The incorporation level decreased greatly from 24 h to 48 and 72 h, indicating that the added ARA had been exhausted or was being consumed at 48 and 72 h. It was also observed that the amount of ARA incorporated into the polar lipid fraction was increased in the fad2 mutant compared to the corresponding wild-type strain (Example 5). It was considered that the improvement may have been due to increased activity of one or more acyltransferases in the fad2 mutant and thereby an increased incorporation rate as the Y. lipolytica cells responded to maintain the ω6 PUFA level in its membranes.

The fad2 mutant is used to test the efficiency of exogenous Δ12 desaturases to convert oleic acid to ω6 fatty acids, as described in Example 16.

TABLE 42
Fatty acid composition of polar lipid and TAG fractions from Y. lipolytica fad2 mutant during culturing in YPD medium with ARA.

| | | | C16: | C16: | | | C18: | C18: | C18: |
|---|---|---|---|---|---|---|---|---|---|
| C14:0 | C15:0 | C16:0 | 1Δ7 | 1Δ9 | C17:1 | C18:0 | 1Δ9 | 1Δ11 | 249, 12 |

No ARA-Polar lipid

| | | | | | | | | | | |
|---|---|---|---|---|---|---|---|---|---|---|
| 24 h | 0.1 | 0.4 | 9.7 | 2.3 | 12.5 | 1.3 | 0.8 | 72.0 | 0.8 | 0.0 |
| 48 h | 0.1 | 0.3 | 8.0 | 4.9 | 12.4 | 1.9 | 0.3 | 71.5 | 0.6 | 0.1 |
| 72 h | 0.0 | 0.2 | 6.0 | 3.6 | 11.5 | 3.3 | 0.2 | 74.5 | 0.5 | 0.0 |

No ARA-Triacylglycerols

| | | | | | | | | | | |
|---|---|---|---|---|---|---|---|---|---|---|
| 24 h | 0.2 | 0.7 | 14.6 | 1.2 | 9.0 | 0.8 | 15.2 | 48.7 | 1.1 | 0.1 |
| 48 h | 0.1 | 0.5 | 14.6 | 2.4 | 8.7 | 0.9 | 11.5 | 53.2 | 1.0 | 0.1 |
| 72 h | 0.1 | 0.4 | 13.1 | 1.8 | 6.9 | 1.5 | 18.0 | 52.0 | 0.8 | 0.0 |

0.1 mg/ml ARA-Polar lipid

| | | | | | | | | | | |
|---|---|---|---|---|---|---|---|---|---|---|
| 24 h | 0.1 | 0.5 | 11.0 | 1.4 | 11.4 | 1.4 | 0.9 | 65.7 | 0.7 | 0.2 |
| 48 h | 0.1 | 0.3 | 8.1 | 4.1 | 11.6 | 2.0 | 0.4 | 71.8 | 0.5 | 0.1 |
| 72 h | 0.0 | 0.2 | 6.1 | 3.3 | 11.5 | 3.1 | 0.2 | 74.6 | 0.5 | 0.0 |

0.1 mg/ml ARA-Triacylglycerols

| | | | | | | | | | | |
|---|---|---|---|---|---|---|---|---|---|---|
| 24 h | 0.2 | 0.7 | 15.8 | 0.7 | 7.8 | 0.8 | 15.5 | 44.1 | 0.8 | 0.2 |
| 48 h | 0.1 | 0.5 | 14.4 | 2.1 | 8.2 | 0.9 | 12.2 | 52.3 | 0.9 | 0.0 |
| 72 h | 0.1 | 0.3 | 12.5 | 1.8 | 6.7 | 1.5 | 19.5 | 50.9 | 0.8 | 0.0 |

0.5 mg/ml ARA-Polar lipid

| | | | | | | | | | | |
|---|---|---|---|---|---|---|---|---|---|---|
| 24 h | 0.3 | 0.9 | 13.9 | 0.5 | 10.1 | 1.1 | 1.4 | 50.8 | 0.5 | 0.3 |
| 48 h | 0.3 | 0.3 | 9.5 | 3.7 | 14.4 | 2.3 | 0.6 | 62.1 | 0.5 | 0.2 |
| 72 h | 0.1 | 0.1 | 5.8 | 1.6 | 19.0 | 4.1 | 0.3 | 66.2 | 0.8 | 0.1 |

0.5 mg/ml ARA-Triacylglycerols

| | | | | | | | | | | |
|---|---|---|---|---|---|---|---|---|---|---|
| 24 h | 0.7 | 1.0 | 17.3 | 0.3 | 8.6 | 0.5 | 14.1 | 34.3 | 0.7 | 0.4 |
| 48 h | 2.5 | 0.3 | 18.0 | 0.7 | 30.3 | 0.4 | 6.5 | 27.5 | 0.5 | 0.1 |
| 72 h | 2.2 | 0.2 | 14.2 | 0.4 | 29.1 | 1.4 | 4.0 | 38.4 | 1.4 | 0.0 |

Fatty acid composition of polar lipid and TAG fractions from Y. lipolytica fad2 mutant during culturing in YPD medium with ARA.

| | | | | | | | | |
|---|---|---|---|---|---|---|---|---|
| C18: | | C20: | C20: | C20: | C20: | | | Lipid % |
| 3ω6 | C20:0 | 1Δ11 | 2Δ11, 14 | 3ω6 | 4ω6 | C22:0 | C24:0 | per CDW |

No ARA-Polar lipid

| | | | | | | | | | |
|---|---|---|---|---|---|---|---|---|---|
| 24 h | 0.0 | 0.0 | 0.0 | 0.0 | 0.0 | 0.0 | 0.0 | 0.1 | 1.8 |
| 48 h | 0.0 | 0.0 | 0.0 | 0.0 | 0.0 | 0.0 | 0.0 | 0.0 | 1.0 |
| 72 h | 0.0 | 0.0 | 0.1 | 0.0 | 0.0 | 0.0 | 0.0 | 0.1 | 1.7 |

No ARA-Triacylglycerols

| | | | | | | | | | |
|---|---|---|---|---|---|---|---|---|---|
| 24 h | 0.0 | 0.7 | 0.0 | 0.0 | 0.0 | 0.0 | 0.7 | 7.2 | 0.3 |
| 48 h | 0.0 | 0.5 | 0.1 | 0.0 | 0.0 | 0.0 | 0.6 | 5.8 | 0.4 |
| 72 h | 0.0 | 0.6 | 0.0 | 0.0 | 0.0 | 0.0 | 0.7 | 4.1 | 0.7 |

0.1 mg/ml ARA-Polar lipid

| | | | | | | | | | |
|---|---|---|---|---|---|---|---|---|---|
| 24 h | 0.2 | 0.0 | 0.0 | 0.0 | 0.9 | 5.5 | 0.0 | 0.1 | 2.0 |
| 48 h | 0.1 | 0.0 | 0.1 | 0.0 | 0.1 | 0.7 | 0.0 | 0.1 | 1.3 |
| 72 h | 0.0 | 0.0 | 0.1 | 0.0 | 0.0 | 0.2 | 0.0 | 0.1 | 1.8 |

0.1 mg/ml ARA-Triacylglycerols

| | | | | | | | | | |
|---|---|---|---|---|---|---|---|---|---|
| 24 h | 0.2 | 0.0 | 0.3 | 0.0 | 1.1 | 4.0 | 0.7 | 7.1 | 0.4 |
| 48 h | 0.0 | 0.6 | 0.1 | 0.0 | 0.2 | 0.5 | 0.7 | 6.3 | 0.5 |
| 72 h | 0.0 | 0.7 | 0.1 | 0.0 | 0.0 | 0.1 | 0.8 | 4.3 | 0.7 |

0.5 mg/ml ARA-Polar lipid

| | | | | | | | | | |
|---|---|---|---|---|---|---|---|---|---|
| 24 h | 0.8 | 0.0 | 0.2 | 0.1 | 2.2 | 17.0 | 0.0 | 0.0 | 1.2 |
| 48 h | 0.3 | 0.0 | 0.1 | 0.0 | 0.5 | 5.2 | 0.0 | 0.0 | 1.1 |
| 72 h | 0.1 | 0.0 | 0.2 | 0.0 | 0.1 | 1.5 | 0.0 | 0.0 | 1.8 |

0.5 mg/ml ARA-Triacylglycerols

| | | | | | | | | | |
|---|---|---|---|---|---|---|---|---|---|
| 24 h | 0.6 | 0.5 | 0.0 | 0.0 | 2.4 | 14.0 | 0.5 | 4.1 | 0.3 |
| 48 h | 0.3 | 0.2 | 0.0 | 0.0 | 1.1 | 9.3 | 0.2 | 2.0 | 0.9 |
| 72 h | 0.2 | 0.1 | 0.1 | 0.0 | 0.8 | 7.7 | 0.0 | 0.0 | 0.8 |

### Example 10. Modification of Y. lipolytica to Generate a Uracil Auxotroph

The URA3 gene of Y. lipolytica encodes the enzyme orotidine-5′-phosphate decarboxylase (EC 4.1.1.23; GenBank Accession No. Q12724), with a variant sequence as Accession No. AJ306421.1 (Mauersberger et al., 2001). The enzyme is required in microbes for synthesis of uracil, so that null mutants in the URA3 gene require the addition of uracil in the medium in order to grow. Such auxotrophic mutants have been used with genetic constructs including a functional URA3 gene as a selectable marker gene, selecting for complementation of the URA3 mutation on defined medium lacking uracil (Mauersberger et al., 2001). Therefore, a URA3 gene deletion mutant was made in Y. lipolytica, starting from the wild-type W29 strain. The strategy used was analogous to that for the fad2KO1 mutant (Example 9, FIG. 5).

Genetic Constructs for Introducing a URA3 Gene Deletion into Y. lipolytica

The nucleotide sequences of the upstream and downstream regions, of 1,000 basepairs each, of the URA3 gene of Y. lipolytica strain W29 were extracted from the NCBI database using the sequence from U40564.1 (www.ncbi.nlm.nih.gov/nuccore/U40564.1/) as a query. The chromosome E sequence was chosen with the identity parameter at 100% and the Changed Region option set to positions 3150692-3154401 to provide a wider range of upstream and downstream sequences. The nucleotide sequence of the URA3 gene is provided as SEQ ID NO: 68 herein including the 1,000 nucleotides upstream of the protein coding sequence and the 1,000 nucleotides downstream. The amino acid sequence of the encoded orotidine-5′-phosphate decarboxylase polypeptide from Y. lipolytica is provided as SEQ ID NO:67.

A DNA fragment corresponding to the 5′ upstream sequence of 1,000 basepairs joined through a SacII restriction enzyme site to the 3′ downstream sequence of 1,000 base pairs was synthesised by GeneArt (Thermofisher, USA), initially in the vector pMK-T, forming pAT069. The DNA fragment had flanking AscI and NotI restriction sites which were used to insert the fragment into a pMK-RQ vector, forming the construct pAT070 (Table 40, Example 9). The nucleotide sequence of the cloned insert was confirmed. By joining the 5′ upstream sequence to the 3′ downstream sequence without an intervening URA3 protein coding sequence, this arrangement effectively deleted the URA3 protein coding sequence of 861 basepairs (ΔURA3).

The DNAs of pAT121 including the hygromycin resistance gene and pAT122 including the nourseothricin resistance gene (Example 9) were digested with SacII and the fragments spanning the genes purified using a gel extraction kit (Qiagen, USA). The DNA fragments were separately ligated with pAT070 which had been digested with SacII and treated with calf intestinal alkaline phosphatase. The ligation mixes were transformed into E. coli DH5α competent cells. DNA was prepared from five colonies for each ligation and DNA samples from the colonies were screened by digestion with the several restriction enzymes and agarose gel electrophoresis to identify and confirm that the correct insertions had occurred between the 5′ upstream and 3′ downstream sequences. The resultant constructs having the Hyg or Nat1 antibiotic resistance gene sequences flanked by the 5′ upstream and 3′ downstream sequences from URA3 were designated pAT257 and pAT258, respectively (Table 40).

### Introduction of URA3 Deletion Constructs

To introduce the genetic construct pAT257 containing the hygromycin resistance gene into Y. lipolytica and identify genetically modified Ura auxotrophic cells from the transformation, the transformation protocol described in Example 9 was followed. Transformed cells were selected on YPD plates containing 250 μg/mL hygromycin. Antibiotic resistant colonies were screened by PCR for the URA3 gene insertion and for uracil auxotrophy. For uracil auxotrophy, the hygromycin resistant colonies were screened on YPD plates and SD-Ura plates, each also containing hygromycin. Colonies that grew on both the YPD and SD-Ura plates were discarded as negatives for the gene deletion, while the colonies that grew on YPD but not on SD-Ura plates were selected as having ura gene deletions. The positive colonies were screened by PCR using primers at270 and at272 (Table 39 of Example 9) with the Phire DNA PCR kit (ThermoFisher). Initial denaturation was at 98° C. for 5 min; followed by 40 cycles of 98° C. for 5 sec, 60° C. for 5 sec and 72° C. for 20 sec per 1 kb, with a final extension of 72° C. for 4 min. Several positives colonies were retested using Taq polymerase with ThermoPol buffer (NEB Biolabs, USA Cat #M0267) to confirm the validity of the gene deletion.

One of the transformed cell lines was retained as the Y. lipolytica ura3 deletion mutant and designated Y. lipolytica strain ura3KO27. This strain was used for introduction of various single-gene (in addition to Ura3 gene) and multi-gene genetic constructs, allowing for selection of the Ura⁺ phenotype.

### Generation of Double Deletion Mutant Fad2-Ura3

The fad2KO1 mutant of Y. lipolytica described in Example 9 was modified in a second round of transformation to introduce a URA3 gene deletion. This used the transformation protocol described above except that pAT258 was used, having the Natl selectable marker gene providing resistance to nourseothricin on YPD medium containing the antibiotic. Colonies that grew on plates containing nourseothricin at a concentration of 400 μg/mL were confirmed as having the ura3 deletion mutation: antibiotic resistant colonies were screened by PCR for the URA3 gene insertion and for uracil auxotrophy, as described above for the ura3KO27 strain. One double mutant strain was retained and designated fad2KO1-ura3KO27. This strain was used for introduction of various single-gene and multi-gene genetic constructs, allowing for selection of the Ura⁺ phenotype in a fad2 mutant background.

### Example 11. Modification of Microbes to Reduce Triacylglycerol Synthesis-Single Gene Mutants

Triacylglycerol (TAG) synthesis in yeasts such as S. cerevisiae and Y. lipolytica occurs by the activity of a suite of enzymes, mostly through the Kennedy pathway, where free fatty acids are firstly linked to coenzyme A (CoA) to produce acyl-CoA molecules. The acyl groups from three acyl-CoAs are then esterified in a step-wise fashion to a glycerol backbone to synthesize TAG. In the first step, glycerol-3-phosphate (G3P) is acylated by a glycerol-3-phosphate acyltransferase (GPAT; EC 2.3.1.15), encoded by the SCT1 and GPT2 genes in S. cerevisiae and the YALI0C00209g gene in Y. lipolytica, to produce lysophosphatidic acid (LPA). LPA is then acylated by lysophosphatidic acid acyltransferase (LPAAT; EC 2.3.1.51; also referred to as 1-acyl-sn-G3P acyltransferase), encoded by the SLC1 gene in S. cerevisiae and the YALI0E18964g gene in Y. lipolytica, to produce phosphatidic acid (PA). This is followed by dephosphorylation of PA by the enzyme phosphatidic acid phosphohydrolase (PAP) to produce diacylglycerol (DAG). In the final step, DAG is acylated by either one of two diacylglycerol acyltransferases (EC 2.3.1.20), DGA1 or DGA2, with acyl-CoA as the acyl donor. DGA1 is encoded by the DGA1 gene in S. cerevisiae and the YALI0E32769g gene in Y. lipolytica. TAG can also be synthesized from DAG by phospholipid: diacylglycerol acyltransferase (PDAT, also known as phospholipid: 1,2-diacyl-sn-glycerol O-acyltransferase; EC 2.3.1.158), encoded by the LRO1 gene in S. cerevisiae and the YALI0E16797g gene in Y. lipolytica, which uses a glycerophospholipid as the acyl donor to produce the TAG. Two different acyl-CoA: sterol acyltransferases (ASAT, EC 2.3.1.26) can also synthesize TAG in S. cerevisiae, encoded by the ARE1 and ARE2 genes, and a single ARE gene, YALI0F06578g, in Y. lipolytica.

Y. lipolytica is considered to be an oleaginous yeast since it can produce more than 20% by weight of lipid (dry cell weight), in some strains up to at least 30% TAG under growth conditions with limited nitrogen. With certain genetic modifications, Y. lipolytica strains can be engineered to produce up to 77% lipid by weight or even more. There are numerous other known oleaginous fungi including other yeasts. In contrast, most strains of S. cerevisiae do not make copious TAG and are not considered to oleaginous yeasts, with the exception of a few strains such as D5A (He et al., 2018).

The present inventors considered that phospholipids containing ω6 fatty acids having at least 3 double bonds could be produced in yeast strains that were genetically modified to produce less TAG. Experiments were therefore designed to inactivate TAG synthesis genes including the DGA1, DGA2, LRO1 and ARE1 genes in Y. lipolytica.

Genetic Constructs for Introducing a DGA1 Gene Deletion into Y. lipolytica

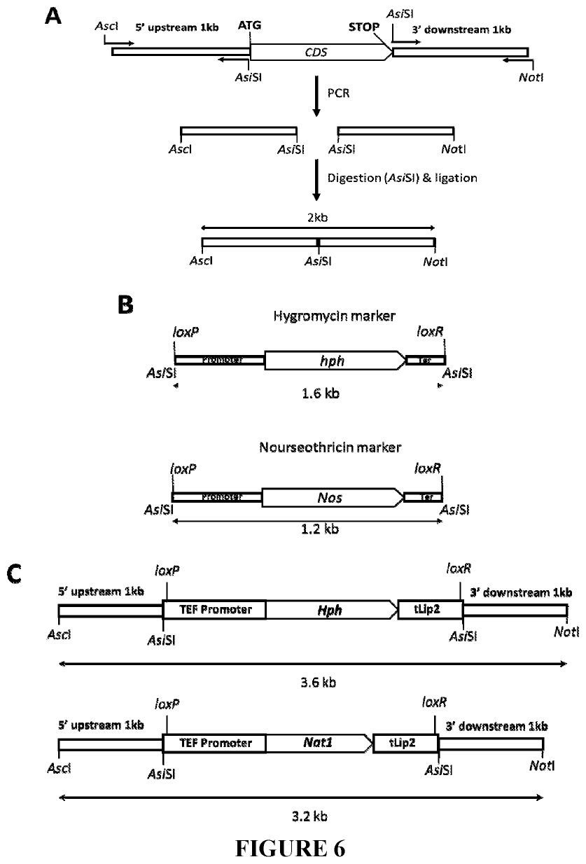

To delete the protein coding sequence of the DGA1 gene and other TAG synthesis genes from the Y. lipolytica genome, thereby providing null mutations, the general strategy described in FIG. 5 for FAD2 was modified in several aspects. A schematic representation of the modified strategy is shown in FIG. 6. As before, the genetic cassette for introducing the gene deletions had the protein coding region of the gene of interest replaced with a selectable marker gene, flanked by 5′ upstream and 3′ downstream sequences which provided for integration of the genetic cassette by recombination into the endogenous gene. This time, however, the 5′ upstream and 3′ downstream sequences of 1,000 basepairs were produced by PCR in-house. Also, the primers used in the amplifications and the selectable marker genes had AsiSI restriction enzyme sites rather than SacII sites.

The nucleotide sequence of the DGA1 gene of Y. lipolytica strain W29 and its upstream and downstream sequences were extracted from the KEGG Yarrowia database (www.genome.jp/kegg-bin/show_organism?org-yli) using the published YALI gene identifier, as gene YALI0E32769p, nucleotides 3885857 to 3889401 of chromosome E, Accession No. CR382131.1. The nucleotide sequence of the DGA1 gene is provided herein as SEQ ID NO:69 including 1,000 nucleotides upstream of the protein coding sequence followed by the protein coding sequence and 1,000 nucleotides downstream of the protein coding sequence.

The amino acid sequence of the encoded DGAT1 polypeptide is provided as SEQ ID NO: 70. Y. lipolytica DGAT1 is a protein of 514 amino acid residues, and is a homolog of animal and plant DGAT2 enzymes. These are all members of the MBOAT protein family (Wang et al., 2013).

The 5′ upstream and 3′ downstream regions adjacent to the DGA1 protein coding region were amplified from genomic DNA from Y. lipolytica strain W29 (FIG. 6). Each amplification reaction used Taq DNA Polymerase with ThermoPol Buffer and a pair of oligonucleotide primers (Table 39 of Example 9). By this means, the 5′ upstream fragment was adapted by adding restriction enzyme sites for AscI at its 5′ end and AsiSI at its 3′ end. Similarly, the 3′ downstream fragment was adapted by adding restriction enzyme sites for AsiSI at its 5′ end and NotI at its 3′ end. (Phusion High Fidelity DNA polymerase, Thermofisher, US) as per manufacturer instructions. The amplified DNA fragments were digested with AsiSI and ligated with T4 DNA Ligase using standard protocols and inserted into vector into the vector pCR Zero Blunt TOPO, forming pAT253 (Table 40 of Example 9). The nucleotide sequence of the cloned insert was confirmed.

The DNAs of pAT123 including the hygromycin resistance gene and pAT124 including the nourseothricin resistance gene (Example 9) were digested with AsiSI and the fragments spanning the genes purified using a gel extraction kit (Qiagen, USA). The DNA fragments were separately ligated with pAT253 DNA which had been digested with AsiSI and treated with calf intestinal alkaline phosphatase. The ligation mixes were transformed into E. coli DH5α competent cells. DNA was prepared from at least five colonies for each ligation and DNA samples from the colonies were screened by digestion with restriction enzymes and agarose gel electrophoresis to identify and confirm that the correct insertions had occurred between the 5′ upstream and 3′ downstream sequences. The resultant constructs having the Hyg or Natl antibiotic resistance gene sequences flanked by the 5′ upstream and 3′ downstream sequences from DGA1 were designated pAT265 and pAT266, respectively (Table 40 of Example 9).

Introduction of DGA1 Deletion Constructs into Y. lipolytica

To introduce the genetic constructs pAT265 containing the Hyg resistance gene and pAT266 containing the Natl resistance gene to replace the DGA1 protein coding region in Y. lipolytica and identify genetically modified Δdga1 cells from the transformation, the transformation protocol described in Example 9 was followed. Transformed cells were selected on YPD plates containing 250 μg/ml hygromycin or 400 μg/ml nourseothricin, according to the selectable marker gene. Antibiotic resistant colonies were screened by PCR for the DGA1 gene insertion. One oligonucleotide primer (at245) located in the 5′ upstream region and a second primer (at247) located within the DGA1 protein coding region were used in PCR reactions to confirm the deletion mutation had been introduced into the genomic DNA of antibiotic resistant colonies. The PCR reaction was performed using Taq DNA polymerase with ThermoPol buffer (NEB, USA) under standard conditions. Lack of an amplification product indicated the presence of the deletion mutation. Genomic DNA from W29 was used in parallel as a positive control for the PCR. A second PCR test using oligonucleotide primers at245 and at248, the latter located in the 3′ region of DGA1, also confirmed the presence of the deletion/insertion mutation, producing a 1.3 kb amplification product in presence of the deletion and a 1.6 kb product in the wild-type, unmutated DGA1. Primer pair at246 and at248 was also used. The absence of the DGA1 protein coding region was confirmed in 7 of 10 colonies tested for the Natl gene.

One of the transformed cell lines from each of the transformations was selected and retained as Y. lipolytica dga1 deletion mutants and designated strains dga1KO1(Hyg) and dga1KO1(Nat1).

The dga1KO1 strains were compared to the corresponding wild-type strain by growth in a high glucose/low nitrogen medium that induces TAG synthesis, to determine the reduction in TAG synthesis ability. A reduction of 50% in the level of TAG is observed in the dga1KO1 mutants after 96 h culturing at 29° C. in the latter medium. The amount of polar lipid and the incorporation of ω6 fatty acids into polar lipids is also assessed by culturing the mutant strains in media containing one or more of the ω6 fatty acids (see below), or by introduction of a genetic construct for production of the ω6 fatty acids (Example 16).

Genetic Constructs for Introducing a DGA2 Gene Deletion into Y. lipolytica

To delete the protein coding sequence of the DGA2 gene from the Y. lipolytica genome, the same strategy was used as for the DGA1 deletion (FIG. 6). The nucleotide sequence of the DGA2 gene of Y. lipolytica strain W29 and its upstream and downstream sequences were extracted from the KEGG Yarrowia database (www.genome.jp/kegg-bin/show_organism?org-yli), using the published YALI gene identifier, as gene YALI0B10153p, Accession No. XP_500707. The nucleotide sequence of the DGA2 gene is provided as SEQ ID NO:71 including 1,000 nucleotides upstream of the protein coding sequence followed by the protein coding sequence and 1,000 nucleotides downstream of the protein coding sequence.

The amino acid sequence of the encoded DGAT2 polypeptide is provided as SEQ ID NO: 72. Y. lipolytica DGAT2 is a protein of 526 amino acid residues. The protein is a member of the membrane-bound O-acyltransferase family-domain-containing (MBOAT) protein with multiple membrane spanning regions, typically 8-10 such regions, that transfer acyl groups to substrates in membranes, in this case to DAG. The Y. lipolytica DGAT2 protein is phylogenetically distinct from the DGAT2s of the ascomycetous yeasts L. kluyveri, K. pastoris, K. lactis, C. albicans, C. parapsilosis and O. polymorpha.

The 5′ upstream and 3′ downstream regions were amplified from genomic DNA from Y. lipolytica strain W29 (FIG. 6). Each amplification reaction used Phusion high fidelity DNA polymerase (NEB, USA) and a pair of oligonucleotide primers (Table 39). As for the amplifications for DGA1, the 5′ upstream fragment had a restriction enzyme site for AscI at its 5′ end and one for AsiSI at its 3′ end. Similarly, the 3′ downstream fragment had a site for AsiSI at its 5′ end and one for NotI at its 3′ end. The amplified DNA fragments were digested with AsiSI, ligated with T4 DNA Ligase, and inserted into the vector pCR Zero Blunt TOPO, forming pAT254 (Table 40 of Example 9). The nucleotide sequence of the cloned insert was confirmed.

The DNAs of pAT123 including the hygromycin resistance gene and pAT124 including the nourseothricin resistance gene (Example 9) were digested with AsiSI and the fragments spanning the genes purified using a gel extraction kit. These were ligated into pAT254 which had been digested with AsiSI and the ligation mixes introduced into E. coli DH5α competent cells. DNA was prepared from five colonies for each ligation and screened by digestion with restriction enzymes. Agarose gel electrophoresis identified the correct constructs and confirmed that the intended insertions had occurred between the 5′ upstream and 3′ downstream sequences. The resultant constructs having the Hyg or Nat1 antibiotic resistance gene sequences flanked by the 5′ upstream and 3′ downstream sequences from DGA2 were designated pAT267 and pAT268, respectively (Table 40 of Example 9).

Introduction of DGA2 Deletion Constructs into Y. lipolytica

To introduce the genetic construct pAT267 containing the hygromycin resistance gene into Y. lipolytica, the transformation protocol described in Example 9 was followed. Transformed cells were selected on YPD plates containing 250 μg/mL hygromycin. Antibiotic resistant colonies were screened by PCR for the DGA2 gene insertion to identify genetically modified Δdga2 cells from the transformation. The oligonucleotide primer pair at249 and at251, internal to the DGA2 protein coding region, were used in PCR reactions to confirm the deletion mutation had been introduced into the genomic DNA of hygromycin resistant colonies. The PCR reaction was performed using Taq DNA polymerase with ThermoPol buffer (NEB, USA) under standard conditions. Lack of an amplification product indicated the presence of the deletion mutation. Genomic DNA from W29 was used in parallel as a positive control for the PCR. A second PCR test using oligonucleotide primers at250 and at252, the latter located in the 3′ region of DGA2, also confirmed the presence of the deletion/insertion mutation. The absence of the DGA2 protein coding region was confirmed in 5 of 6 colonies selected with the Hyg gene.

One of the transformed cell lines was retained as a Y. lipolytica dga2 deletion mutant and designated Y. lipolytica strain dga2KO1(Hyg).

The strain dga2KO1(Hyg) was compared to its corresponding wild-type strain by growth in a high glucose/low nitrogen medium (DM-Glyc-LowN) that induces TAG synthesis, to determine the reduction in TAG synthesis ability. A reduction in the level of TAG of about 46% was observed in the dga2KO1 mutant compared to the wild-type DGA2 strain. The amount of polar lipid and the incorporation of ω6 fatty acids into polar lipids is also assessed by culturing the mutant strain in media containing one or more of the ω6 fatty acids (see below), or by introduction of a genetic construct for production of the ω6 fatty acids (Example 16).

Genetic Constructs for Introducing a LRO1 Gene Deletion into Y. lipolytica

To delete the protein coding sequence of the LRO1 gene from the Y. lipolytica genome, the same strategy was used as for the DGA1 deletion (FIG. 6). The nucleotide sequence of the LRO1 gene of Y. lipolytica strain W29 and its upstream and downstream sequences were extracted from the KEGG Yarrowia database (www.genome.jp/kegg-bin/show_organism?org-yli), using the published YALI gene identifier, as gene YALI0E16797p, in Accession No. CR382131.1. The nucleotide sequence of the LRO1 gene is provided as SEQ ID NO:73 including 1,000 nucleotides upstream of the protein coding sequence followed by the protein coding sequence and 1,000 nucleotides downstream of the protein coding sequence.

The amino acid sequence of the encoded PDAT polypeptide is provided as SEQ ID NO: 74. Y. lipolytica PDAT encoded by the LRO1 gene is a protein of 648 amino acid residues.

The 5′ upstream and 3′ downstream regions adjacent to the PDAT protein coding region were amplified from genomic DNA from Y. lipolytica strain W29 using Phusion high fidelity DNA polymerase (NEB, USA) and a pair of oligonucleotide primers (Table 39 of Example 9). As for the amplifications for DGA1, the 5′ upstream fragment had a restriction enzyme site for AscI at its 5′ end and one for AsiSI at its 3′ end. Similarly, the 3′ downstream fragment had a site for AsiSI at its 5′ end and one for NotI at its 3′ end. The amplified DNA fragments were digested with AsiSI, ligated with T4 DNA Ligase, and inserted into the vector pCR Zero Blunt TOPO, forming pAT256 (Table 40 of Example 9). The nucleotide sequence of the cloned insert was confirmed.

The DNAs of pAT123 including the hygromycin resistance gene and pAT124 including the nourseothricin resistance gene (Example 9) were digested with AsiSI and the fragments spanning the genes purified using a gel extraction kit. These were ligated into pAT256 which had been digested with AsiSI and the ligation mixes introduced into E. coli DH5α competent cells. DNA was prepared from five colonies for each ligation and screened by digestion with restriction enzymes. The correct constructs were identified by agarose gel electrophoresis, confirming that the intended insertions had occurred between the 5′ upstream and 3′ downstream sequences. The resultant constructs having the Hyg or Natl antibiotic resistance gene sequences flanked by the 5′ upstream and 3′ downstream sequences from LRO1 were designated pAT271 and pAT272, respectively (Table 40 of Example 9).

Introduction of LRO1 Deletion Constructs into Y. lipolytica

To introduce the genetic construct pAT272 containing the nourseothricin resistance gene into Y. lipolytica, the transformation protocol described in Example 9 was followed. Transformed cells were selected on YPD plates containing 400 μg/mL nourseothricin. Antibiotic resistant colonies were screened by PCR for the LRO1 gene insertion to identify genetically modified Δlro1 cells from the transformation. One oligonucleotide primer (at257) located in the 5′ upstream region and a second primer (at260) located in the 3′ downstream region of LRO1 were used in PCR reactions to confirm the deletion mutation had been introduced into the genomic DNA of antibiotic resistant colonies. The PCR reaction was performed using Taq DNA polymerase with ThermoPol buffer (NEB, USA) under standard conditions. DNA from W29 was used in parallel as a positive control for the PCR. Lack of a 2.2 kb amplification product and the presence of a 1.4 kb product indicated the presence of the deletion mutation. Confirmatory PCR reactions were carried our using primers at258 and at260. The absence of the LRO1 protein coding region was confirmed in four of ten colonies tested.

One oligonucleotide primer (at245) located in the 5′ upstream region and a second primer (at247) located within the DGA1 protein coding region were used in PCR reactions to confirm the deletion mutation had been introduced into the genomic DNA of antibiotic resistant colonies. The PCR reaction was performed using Taq DNA polymerase with ThermoPol buffer (NEB, USA) under standard conditions. Lack of an amplification product indicated the presence of the deletion mutation. Genomic DNA from W29 was used in parallel as a positive control for the PCR. A second PCR test using oligonucleotide primers at245 and at248, the latter located in the 3′ region of DGA1, also confirmed the presence of the deletion/insertion mutation, producing a 1.3 kb amplification product in presence of the deletion and a 1.6 kb product in the wild-type, unmutated DGA1. The absence of the LRO1 protein coding region was confirmed in 4 of 10 colonies tested for the Natl gene.

One of the transformed cell lines was retained as a Y. lipolytica lro1 deletion mutant and designated Y. lipolytica strain lro1KO1.

The strain lro1KO1 was compared to its corresponding wild-type strain by growth in a high glucose/low nitrogen medium (DM-Glyc-LowN) that induces TAG synthesis, to determine the reduction in TAG synthesis ability. A reduction in the level of TAG was observed in the lro1KO1 mutant, by about 30%. The percentage of the total saturated fatty acids decreased from about 55% in wild-type strain W29 to about 50% in the lro1 mutant, so less of a change than in the dga1 and dga2 mutants. The amount of polar lipid and the incorporation of ω6 fatty acids into polar lipids is also assessed by culturing the mutant strain in media containing one or more of the ω6 fatty acids (below), or by introduction of a genetic construct for production of the ω6 fatty acids (Example 16).

Genetic Constructs for Introducing an ARE1 Gene Deletion into Y. lipolytica

The genes ARE1 and ARE2 in fungi, including S. cerevisiae, encode the enzyme acyl-CoA: sterol acyltransferases (ASAT, EC 2.3.1.26) which can also synthesize TAG. Y. lipolytica appears to have a single ARE gene, namely ARE1. To delete the protein coding sequence of the ARE1 gene from the Y. lipolytica genome, the same strategy was used as for the DGA1 deletion (FIG. 6). The nucleotide sequence of the ARE1 gene of Y. lipolytica strain W29 and its upstream and downstream sequences were extracted from the KEGG Yarrowia database (www.genome.jp/kegg-bin/show_organism?org-yli), using the published YALI gene identifier, as gene YALI0F06578g, in Accession No. CR382131.1. The nucleotide sequence of the ARE1 gene is provided as SEQ ID NO:75 including 1,000 nucleotides upstream of the protein coding sequence followed by the protein coding sequence and 1,000 nucleotides downstream of the protein coding sequence.

The amino acid sequence of the encoded ASAT polypeptide is provided as SEQ ID NO: 76. Y. lipolytica ASAT is a protein of 543 amino acid residues.

The 5′ upstream and 3′ downstream regions adjacent to the ASAT protein coding region were amplified from genomic DNA from Y. lipolytica strain W29 (FIG. 6). Each amplification reaction used Phusion high fidelity DNA polymerase (NEB, USA) and a pair of oligonucleotide primers (Table 39 of Example 9). As for the amplifications for DGA1, the 5′ upstream fragment had a restriction enzyme site for AscI at its 5′ end and one for AsiSI at its 3′ end. Similarly, the 3′ downstream fragment had a site for AsiSI at its 5′ end and one for NotI at its 3′ end. The amplified DNA fragments were digested with AsiSI, ligated with T4 DNA Ligase, and inserted into the vector pCR Zero Blunt TOPO, forming pAT251 (Table 40 of Example 9). The nucleotide sequence of the cloned insert was confirmed.

The DNAs of pAT123 including the hygromycin resistance gene and pAT124 including the nourseothricin resistance gene (Example 9) were digested with AsiSI and the fragments spanning the genes purified using a gel extraction kit. These were ligated into pAT251 which had been digested with AsiSI and the ligation mixes introduced into E. coli DH5α competent cells. DNA was prepared from five colonies for each ligation and screened by digestion with restriction enzymes. The correct constructs were identified by agarose gel electrophoresis, confirming that the intended insertions had occurred between the 5′ upstream and 3′ downstream sequences. The resultant constructs having the Hyg or Nat1 antibiotic resistance gene sequences flanked by the 5′ upstream and 3′ downstream sequences from LRO1 were designated pAT261 and pAT262, respectively (Table 40 of Example 9).

Introduction of ARE1 Deletion Constructs into Y. lipolytica

To introduce the genetic constructs pAT261 and pAT262 into Y. lipolytica, the transformation protocol described in Example 9 was followed. Transformed cells were selected on YPD plates containing the appropriate antibiotic. Antibiotic resistant colonies were screened by PCR for the ARE1 gene insertion to identify genetically modified Δare1 cells from the transformation. DNA from W29 was used in parallel as a positive control for the PCRs. Primer pair at241 and at244 located in the 5′ upstream region and the 3′ downstream region, respectively, were used in PCR reactions to confirm the deletion mutation had been introduced into the genomic DNA of antibiotic resistant colonies. Additional PCR reactions with primer pairs at242 and at243, internal in the protein coding region, and at242 and at244 confirmed the presence of the deletion/insertion mutations. The absence of the ARE1 protein coding region was confirmed in 3 of 6 colonies resistant to hygromycin and 3 of 4 colonies resistant to nourseothricin. One of each of the transformed cell lines were retained as Y. lipolytica are1 deletion mutants and designated Y. lipolytica strain are1KO1(Hyg) and are1KO1(Nat1).

The are1KO1 strains are compared to the corresponding wild-type strain by growth in YPD, a rich medium, and a high glucose/low nitrogen medium that induces TAG synthesis, to determine the reduction in TAG synthesis ability. No great reduction in the level of TAG is observed in the are1KO1 mutants in the latter medium. The amount of polar lipid and the incorporation of ω6 fatty acids into polar lipids is also assessed by culturing the mutant strain in media containing one or more of the ω6 fatty acids, or by introduction of a genetic construct for production of the ω6 fatty acids (Example 16).

Assessment of ARA Incorporation into Lipids Following ARA-Feeding of Lro1KO, Dga1KO and Dga2KO Y. Lipolytica Strains

Wild-type (W29) as well as lro1KO, dga1KO and dga2KO Y. lipolytica strains were cultured in batch medium YPD (Yeast extract 10 g/L, peptone 20 g/L, glucose 20 g/L) essentially as described above. This rich media allows for high biomass growth and high PL yield. Briefly, three colonies of each strain were inoculated into 250 ml flask with 50 mL YPD at 28° C. and cultured shaking at 150 rpm for 24 hours. Fermentation flasks were inoculated with the seed cultures and cultivated at 28° C. with 150 rpm shaking for 15 hours. ARA (Nuchek arachidonic acid, pure free fatty acid) was fed to the cultures when the OD was 0.3, at initial concentration of 2 mg/ml. A negative control with no ARA feeding was also included. Biomass was harvested for lipid analysis.

As shown in Table 43, ARA incorporation was approximately 3 times higher in the PL fractions of the lro1KO, dga1KO and dga2KO strains compared to wild-type. The ratio of PL to TAG also increased up to about 3-fold in the mutants, such as the dga1KO.

| | | | | | | | | | | | | | Ratio |
|---|---|---|---|---|---|---|---|---|---|---|---|---|---|
| | | | | | | | | | | | | | PL |
| | | | | | | | | | | | | | to |
| | | | C16:0 | C16:1 | C18:0 | C18:1 | C18:2 | C18:3 | C20:0 | C22:0 | ARA | C24:0 | TAG |
| TAG- | non-fed | w29 | 11.63 | 10.95 | 5.10 | 46.23 | 26.09 | 0.00 | 0.00 | 0.00 | 0.00 | 0.00 | |
| fraction | fed | w29 | 8.82 | 13.38 | 0.00 | 56.78 | 21.02 | 0.00 | 0.00 | 0.00 | 0.00 | 0.00 | |
| PL- | non-fed | w29 | 10.38 | 12.58 | 0.00 | 48.93 | 28.10 | 0.00 | 0.00 | 0.00 | 0.00 | 0.00 | 1.78 |
| fraction | fed | w29 | 12.43 | 10.77 | 0.00 | 48.83 | 21.19 | 0.00 | 0.00 | 0.00 | 6.78 | 0.00 | |
| TAG- | non-fed | dgal | 8.78 | 12.65 | 0.00 | 41.65 | 36.92 | 0.00 | 0.00 | 0.00 | 0.00 | 0.00 | |
| fraction | fed | dgal | 9.17 | 7.92 | 0.00 | 32.43 | 15.32 | 0.00 | 0.00 | 0.00 | 35.15 | 0.00 | |
| PL- | non-fed | dgal | 11.77 | 11.88 | 0.00 | 42.27 | 34.08 | 0.00 | 0.00 | 0.00 | 0.00 | 0.00 | 2.99 |
| fraction | fed | dgal | 17.15 | 8.61 | 0.55 | 36.93 | 16.49 | 0.64 | 0.00 | 0.00 | 18.83 | 0.00 | |
| TAG- | non-fed | dga2 | 9.64 | 11.47 | 3.66 | 40.34 | 34.88 | 0.00 | 0.00 | 0.00 | 0.00 | 0.00 | |
| fraction | fed | dga2 | 10.82 | 8.66 | 2.67 | 37.17 | 13.91 | 0.00 | 0.00 | 0.00 | 26.76 | 0.00 | |
| PL- | non-fed | dga2 | 11.97 | 12.58 | 0.00 | 39.76 | 35.69 | 0.00 | 0.00 | 0.00 | 0.00 | 0.00 | 2.06 |
| fraction | fed | dga2 | 16.73 | 10.07 | 0.00 | 39.06 | 16.89 | 0.00 | 0.00 | 0.00 | 16.58 | 0.00 | |
| TAG- | non-fed | lro1 | 20.65 | 0.00 | 0.00 | 49.26 | 30.09 | 0.00 | 0.00 | 0.00 | 0.00 | 0.00 | |
| fraction | fed | lro1 | 10.57 | 8.45 | 3.45 | 34.59 | 10.00 | 0.00 | 0.00 | 0.00 | 32.93 | 0.00 | |
| PL- | non-fed | lrol | 15.70 | 8.94 | 0.00 | 42.64 | 32.72 | 0.00 | 0.00 | 0.00 | 0.00 | 0.00 | 1.53 |
| fraction | fed | lro1 | 15.34 | 9.30 | 0.00 | 42.67 | 15.61 | 0.00 | 0.00 | 0.00 | 16.30 | 0.00 | |

### Example 12. Modification of Microbes to Reduce Triacylglycerol Synthesis-Multi Gene Mutants

As described in Example 11, single gene mutants were produced in Y. lipolytica that had deletions in any one of four genes for TAG biosynthesis, namely DGA1, DGA2, LRO1 and ARE1. The inventors now aimed to produce mutants having multiple gene deletions, to further decrease TAG synthesis and reduce the TAG: PL ratio. This involved the removal first of all of an antibiotic resistance marker gene, for example the nourseothricin resistance gene, to allow re-use of the marker gene in a subsequent transformation.

### Cre-Lox Excision of Selectable Marker Genes

Where the selectable marker gene other than a hygromycin resistance gene was flanked by lox sites, the plasmid pUB4-CRE is used. This vector encodes a Cre recombinase protein which can excise the DNA between two lox sites. pUB4-CRE, which is a replicative vector from strain JME547, was obtained from INRAE, France (Fickers et al., 2003). The S. cerevisiae or Y. lipolytica strains to be modified are transformed with the plasmid pUB4-CRE as described below, selecting for hygromycin resistance. Colonies are plated on media with and without nourseothricin to screen for loss of the selectable marker gene. Colonies which are sensitive to the antibiotic are selected and the loss of the nourseothricin gene confirmed by PCR with flanking and internal primer combinations, and sequencing of the deletion region. A selected colony is grown in YPD medium in the absence of hygromycin i.e. without selection pressure and plated to identify a colony that has lost pUB4-CRE (Fickers et al., 2003). Such a colony is selected as the strain from which the nourseothricin selectable marker gene has been excised. An analogous procedure is followed for excision of a hygromycin resistance selectable marker gene using a derivative of pUB4-CRE having a selectable marker gene other than the hygromycin resistance gene.

The transformation procedure to introduce pUB4-CRE is as follows using a Frozen-EZ Yeast Transformation II kit (Zymo Research, USA). The 50 μl of Ura-KO21 competent cells, prepared as per instructions from Zymo Research are transformed with 0.5-1 μg DNA in a 5 to 10 μl volume. Then 500 μl EZ 3 solution is added mixed thoroughly with the cell suspension. The mix is incubated at 30° C. for between 45 min and 2 h, with occasional gentle mixing. 50-150 μl of the transformation mixture is spread on a YPD plate having hygromycin and lacking nourseothricin. The plates are incubated at 30° C. for 2-4 days to allow for growth of transformants.

### Generation of Mutants Having Multiple Inactivating Mutations

Once the hygromycin or nourseothricin resistance marker gene is excised from the mutated gene, for example the DGA1 gene, and the pUB4-CRE excision plasmid has been lost from the cells, the hygromycin or nourseothricin selectable marker gene can be used again in a second round of mutagenesis to inactivate a second gene. The process for marker excision can be repeated and a third round of mutagenesis carried out on a third gene, followed by fourth cycle of mutagenesis. With this strategy, double, triple and finally the quadruple mutants are generated for all combinations of the four genes. Preferred mutants for production of polar lipids containing ω6 fatty acids are the double mutants dga1-dga2 and dga1-lro1, the triple mutant dga1-dga2-lro1 and the quadruple mutant dga1-dga2-lro1-are1.

A similar strategy is followed with S. cerevisiae strain D5A to inactivate multiple genes selected from DGA1, DGA2, LRO1 and ARE1. Preferred Y. lipolytica and S. cerevisiae strains of the D5A type for production of polar lipids containing ω6 fatty acids are the double mutants dga1-dga2 and dga1-lro1, the triple mutant dga1-dga2-lro1 and the quadruple mutant dga1-dga2-lro1-are1.

### Example 13. Production of Neutral and Polar Lipids in Single and Multiple Gene Mutants

Each of the Y. lipolytica and S. cerevisiae mutants generated as described in Examples 11 and 12 are grown in a medium having a high glucose content and low nitrogen content to induce TAG production. A decrease in TAG production is observed in all of the mutant strains with the possible exception of the are1 single gene mutant. An even further reduced level of TAG production is observed in the double and triple mutants compared to the single gene mutants. Lipid is extracted from the harvested cells and fractionated into polar lipid and neutral lipid. An increased ratio of polar lipid: neutral lipid is observed. The polar lipid is fractionated into the PL classes and the relative amounts of PE, PC, PS, PI and PG are observed.

### Example 14. Production of Neutral and Polar Lipids in Single and Multiple Gene Mutants

The mutant Y. lipolytica and S. cerevisiae strains are also grown in YPD medium and in the high glucose medium, each with and without ω6 fatty acid supplementation, in particular supplementation with one or more of DGLA, ARA, DTA and DPA-ω6, to measure the incorporation of the ω6 fatty acids into polar lipids. Lipid is extracted from the harvested cells and fractionated into polar lipid and neutral lipid, and the fatty acid composition determined for each growth condition. The mutants exhibit a greater amount of the ω6 fatty acids incorporated into the polar lipid compared to the parental strains which are wild-type for DGA1, DGA2, LRO1 and ARE1, for both media. An increase is observed in the absolute amount of polar lipid produced in the mutant strains, including in PL, as well as an increase in the PUFA content relative to the sum of the saturated fatty acid content and the monounsaturated fatty acid content. For example, an increased efficiency of 412 desaturation is observed in the multi-gene mutants relative to the corresponding wild-type strain.

### Example 15. Genetic Modification of Microbes that Produce Omega-6 Fatty Acids

Several laboratories have reported the engineering of S. cerevisiae or Y. lipolytica for production of PUFA, in particular for production of ω3 fatty acids such as EPA. Those reports, however, aimed to produce the PUFA in TAG rather than in phospholipid. The inventors therefore sought to modify several of these strains to produce less TAG and a higher ratio of polar lipid to neutral lipid.

WO2006/055322 describes a Y. lipolytica strain designated Y2047 which was reported to produce up to 11% ARA as a weight percentage of its total fatty acid content, which was almost entirely in the form of TAG. This strain was transformed with two genetic constructs. The first construct encoded a Δ12 desaturase from Fusarium moniliforme, a Δ6 desaturase from Mortierella alpina, and two fatty acid elongases, namely one from M. alpina and one from Thraustochytrium aureum; this construct was integrated into the URA3 gene of Y. lipolytica. The second construct had three genes each encoding a Δ5 desaturase, namely two genes encoding the same Δ5 desaturase from M. alpina and one gene encoding a Δ5 desaturase from Homo sapiens; this construct was integrated into the LEU2 gene of Y. lipolytica. WO2006/055322 did not describe the fatty acid composition of this strain other than reporting about 11% ARA and about 25-29% GLA in the total fatty acid content.

Strain Y2096 (U.S. Pat. No. 7,932,077) had the two same constructs as Y2047 but had five additional constructs, namely a construct pZP3L37 having three genes each encoding the Saprolegnia diclina Δ17 desaturase, inserted into the POX3 gene, a construct pZKUT16 having a gene encoding a rat fatty acid elongase, a construct pKO2UM25E having genes encoding a fatty acid elongase from M. alpina, a M. isabellina Δ12 desaturase and a Δ5 desaturase gene from Isochrysis galbana, inserted into the Yarrowia Δ12 desaturase gene, a construct pZKUGPI5S having two genes each encoding a Δ5 desaturase, integrated into the URA3 gene, and a construct pDMW303 having four genes encoding a C18/20 elongase, a Δ6 desaturase, a Δ5 desaturase and a Δ12 desaturase. Strain Y2096 produced up to about 28% EPA under optimal conditions for production of TAG containing EPA. U.S. Pat. No. 7,932,077 did not describe the fatty acid composition of this strain other than reporting about 24-28% EPA in the total fatty acid content when conditions were optimal for producing TAG.

Strains Y2047 and Y2096 were obtained from ATCC and cultured in YPD medium. Cells were harvested at 24 and 48 h and the polar lipid and TAG fractions isolated from the total extracted lipid in the cells. The fatty acid composition of the polar lipid and TAG fractions was determined by GC analysis of FAME. The data are shown in Table 43. The lipid fractions from Y2047 and Y2096 contained ARA at levels of 1.8-2.7% and EPA at levels of 2.6-5.6%, respectively, i.e. much lower than the reported levels under conditions to optimise TAG production. All of the fractions contained much more GLA in the range of 11.7-24.8% than DGLA or ARA, or the sum of DGLA and ARA, indicating low efficiency for elongation of GLA to DGLA.

Single, double and triple knockout mutations are introduced into the DGA1, DGA2 and LRO1 genes of strains Y2047 and Y2096 to reduce the synthesis of TAG relative to polar lipids. These mutants are grown in YPD medium and in a medium containing high levels of glucose and low levels of nitrogen. Polar lipids are extracted from these cells after 24 and 48 h culturing. When grown in the high glucose/low nitrogen medium, the fatty acid composition of the polar lipids show that the mutant cells have an increased efficiency of conversion of LA to GLA and GLA to DGLA and ARA compared to the parental strains Y2047 and Y2096.

TABLE 43
Fatty acid composition of polar lipid and TAG fractions from Y. lipolytica strains producing ARA or EPA.

| | | | | | | | | | | |
|---|---|---|---|---|---|---|---|---|---|---|
| | | | | C16: | C16: | | | C18: | C18: | C18: |
| C12:0 | C14:0 | C15:0 | C16:0 | 1Δ7 | 1Δ9 | C16:2? | C18:0 | 1Δ9 | 1Δ11 | 2Δ9, 12 |

Y2047-Polar lipid

| | | | | | | | | | | | |
|---|---|---|---|---|---|---|---|---|---|---|---|
| 24 h | 0.0 | 0.3 | 1.2 | 17.7 | 0.4 | 8.1 | 2.3 | 2.5 | 11.5 | 1.0 | 27.3 |
| 48 h | 0.0 | 0.4 | 1.4 | 22.5 | 0.4 | 11.9 | 1.8 | 4.1 | 22.4 | 1.4 | 0.0 |

Y2047-Triacylglycerols

| | | | | | | | | | | | |
|---|---|---|---|---|---|---|---|---|---|---|---|
| 24 h | 0.0 | 0.4 | 1.0 | 15.4 | 0.3 | 7.2 | 2.5 | 11.8 | 12.2 | 1.3 | 15.8 |
| 48 h | 0.0 | 0.4 | 1.0 | 19.4 | 0.3 | 6.6 | 1.4 | 16.4 | 11.5 | 1.2 | 11.2 |

Y2096-Polar lipid

| | | | | | | | | | | | |
|---|---|---|---|---|---|---|---|---|---|---|---|
| 24 h | 0.1 | 0.9 | 1.1 | 24.2 | 0.0 | 1.6 | 1.3 | 8.3 | 4.9 | 1.6 | 17.4 |
| 48 h | 0.0 | 1.5 | 0.8 | 23.9 | 0.0 | 3.2 | 0.9 | 10.2 | 17.4 | 1.4 | 0.0 |

Y2096-Triacylglycerols

| | | | | | | | | | | | |
|---|---|---|---|---|---|---|---|---|---|---|---|
| 24 h | 0.1 | 0.7 | 0.9 | 12.0 | 0.0 | 1.3 | 1.4 | 19.2 | 7.8 | 1.7 | 19.2 |
| 48 h | 0.1 | 1.0 | 0.4 | 11.5 | 0.0 | 1.4 | 0.7 | 23.5 | 8.5 | 1.1 | 19.1 |

Fatty acid composition of polar lipid and TAG fractions from Y. lipolytica strains producing ARA or EPA.

| | | | | | | | | | |
|---|---|---|---|---|---|---|---|---|---|
| C18: | C18: | | C20: | C20: | C20: | C20: | | | C22: |
| 3ω6 | 3ω3 | C20:0 | 1Δ11 | 2Δ11, 14 | 3ω6 | 4ω6 | C22:0 | C24:0 | 5ω3 |

Y2047-Polar lipid

| | | | | | | | | | | |
|---|---|---|---|---|---|---|---|---|---|---|
| 24 h | 22.1 | 0.0 | 0.1 | 0.3 | 0.3 | 2.6 | 2.0 | 0.0 | 0.0 | 0.0 |
| 48 h | 24.8 | 0.0 | 0.1 | 0.6 | 0.4 | 5.0 | 2.1 | 0.0 | 0.0 | 0.1 |

Y2047-Triacylglycerols

| | | | | | | | | | | |
|---|---|---|---|---|---|---|---|---|---|---|
| 24 h | 19.6 | 0.0 | 0.7 | 0.4 | 0.4 | 2.9 | 2.7 | 0.0 | 0.7 | 0.0 |
| 48 h | 16.5 | 0.0 | 0.9 | 0.5 | 0.3 | 4.1 | 1.8 | 0.0 | 1.0 | 0.0 |

Y2096-Polar lipid

| | | | | | | | | | | |
|---|---|---|---|---|---|---|---|---|---|---|
| 24 h | 24.0 | 0.2 | 0.1 | 0.1 | 0.1 | 3.3 | 0.0 | 0.3 | 0.0 | 3.1 |
| 48 h | 21.0 | 0.3 | 0.2 | 0.3 | 0.2 | 4.8 | 0.0 | 0.4 | 0.1 | 5.6 |

Y2096-Triacylglycerols

| | | | | | | | | | | |
|---|---|---|---|---|---|---|---|---|---|---|
| 24 h | 17.0 | 0.1 | 0.9 | 0.1 | 0.1 | 3.6 | 0.8 | 0.3 | 0.6 | 2.6 |
| 48 h | 11.7 | 0.1 | 1.2 | 0.1 | 0.2 | 3.7 | 0.6 | 0.3 | 1.3 | 3.8 |

### Example 16. Genetic Constructs for Producing Omega-6 Fatty Acids

Yeast cells do not naturally produce ω6 fatty acids other than LA and sometimes C20:2Δ11,14, and some species such as S. cerevisiae do not even produce LA. Several laboratories have engineered S. cerevisiae or Y. lipolytica for production of PUFA, in particular for production of ω3 fatty acids such as EPA. Those reports, however, aimed to produce the PUFA in TAG rather than in phospholipid. The inventors therefore designed a series of genetic constructs for the production of ω6 fatty acids in yeast cells, for example S. cerevisiae or Y. lipolytica, through a combination of fatty acid desaturases and elongases. For yeast cells that produce LA endogenously, production of GLA requires a Δ6 desaturase, while production of the C20:3 fatty acid DGLA requires either a Δ6 desaturase and a Δ6 elongase or a Δ9 elongase combined with a Δ8 desaturase, or both pairs of enzymes. The use of a Δ9 elongase alone provides for production of the ω6 fatty acid C20:2Δ11,14 (EDA) from LA. Production of ARA from DGLA requires the addition of a Δ5 desaturase, and production of DTA from ARA requires a further addition of a Δ5 elongase. Finally, if production of DPAω6 (C22:5Δ4,7,10,13,16) from DTA is desired, a Δ4 desaturase must be added as a further enzyme. For yeast cells such as S. cerevisiae that do not naturally produce LA, the enzymatic pathway must include a Δ12 desaturase to first of all convert oleic acid to LA. Even for yeast cells that naturally produce LA, the present inventors predicted that addition of an exogenous Δ12 desaturase will increase the amount of LA available in the cells to be converted to the desired ω6 fatty acid product. For example, one or more copies of a gene encoding the endogenous Δ12 desaturase are added exogenously to the yeast cells.

Another consideration was the choice of each of these fatty acid desaturase and elongase enzymes from the many known enzymes with the required activity, in particular considering whether the desaturases work on an acyl-CoA substrate or an acyl-lipid substrate such as an acyl group esterified in the form of PC, or both. The inventors concluded that enzymes that function on acyl-CoA substrates were preferred for an efficient pathway and increasing the amount of ω6 product formed. One exception to this preference, however, was the choice of Δ6 desaturase where GLA was the desired product or the Δ5 desaturase if ARA was the desired final product or the Δ4 desaturase if DPA-ω6 was the desired final product. In those instances, an acyl-lipid type desaturase should function well in producing GLA, ARA or DPA-ω6, respectively, esterified to phospholipid as the final product. Another factor that was considered was the activity of each enzyme on an ω6 substrate relative to a corresponding ω3 substrate.

Based on these factors, the enzymes selected are shown in Table 44.

TABLE 44
Preferred enzymes for synthesis of ω6 fatty acids in yeast cells or other fungi.

| | |
|---|---|
| Δ12 desaturase | Lachancea kluyveri Δ12 desaturase (Accession No. BAD08375.1, |
| | SEQ ID NO: 1); Y. lipolytica Δ12 desaturase (Accession No. |
| | XP_500707.1; SEQ ID NO: 2); Acheta domesticus Δ12 desaturase |
| | (Accession No. ABY26957.1, SEQ ID NO: 3); Fusarium moniliforme |
| | Δ12 desaturase (Accession No. XP_018751050.1; SEQ ID NO: 4) |
| Δ6 desaturase | Ostreococcus tauri (Accession No. XP_003082578.1; SEQ ID NO: 5); |
| | Mortierella alpina (Accession No. AAL73949.1; SEQ ID NO: 6) |
| Δ6 elongase | Pyramimonas cordata (Accession No. ACR53359.1; SEQ ID NO: 13) |
| Δ9 elongase | Pavlova pinguis (Accession No.; SEQ ID NO: 7); |
| | Isochrysis galbana (Accession No. ADD51571; SEQ ID NO: 11) |
| Δ8 desaturase | Pavlova salina (Accession No. A4KDP1.1; SEQ ID NO: 14) |
| Δ5 desaturase | Pavlova salina (Accession No. A4KDP0.1; SEQ ID NO: 15); |
| | M. alpina Δ5 desaturase (Accession No.; SEQ ID NO: 16) |
| Δ5 elongase | Pyramimonas cordata (Accession No. ACR53360.1; SEQ ID NO: 17) |
| Δ4 desaturase | Pavlova salina (Accession No. A0PJ29.1; SEQ ID NO: 18); |
| | Thraustochytrium (Accession No. CAX48933; SEQ ID NO: 19) |

In order to test different Δ12 desaturases for synthesis of LA from oleic acid in yeast cells or other microbes, the inventors designed a series of genetic constructs with four different Δ12 desaturases under the control of one or other of two different promoters, for expression in either S. cerevisiae or Y. lipolytica. For Y. lipolytica:
Construct A1: Encoding the L. kluyveri Δ12 desaturase, under the control of the pFBAINm promoter.
Construct A2: Encoding the L. kluyveri Δ12 desaturase, under the control of the pTEF promoter.
Construct B1: Encoding the Y. lipolytica Δ12 desaturase, under the control of the pFBAINm promoter.
Construct B2: Encoding the Y. lipolytica Δ12 desaturase, under the control of the pTEF promoter.
Construct C1: Encoding the A. domesticus Δ12 desaturase, under the control of the pFBAINm promoter.
Construct C2: Encoding the A. domesticus Δ12 desaturase, under the control of the pTEF promoter.
Construct D1: Encoding the F. moniliforme Δ12 desaturase, under the control of the pFBAINm promoter.
Construct D2: Encoding the F. moniliforme Δ12 desaturase, under the control of the pTEF promoter.

For S. cerevisiae, a corresponding set of eight constructs was designed using a pPGK promoter instead of the pFBAINm promoter and a ENO1 or TDH3 gene promoter instead of the pTEF promoter. In each case, the protein coding regions were codon optimised for either Y. lipolytica or S. cerevisiae. Each construct included an optimised translation start site immediately before the translation start codon ATG. In the case of Y. lipolytica, the constructs are inserted between the 5′ and 3′ flanking regions of the POX2 gene (SEQ ID NO:77), whereas for S. cerevisiae, the flanking regions were from the POX1 (YGL205w) gene (SEQ ID NO:79), in each case to provide for insertion of the expression cassettes into that gene with resultant inactivation of the endogenous gene. The constructs also have a selectable marker gene between the flanking POX2 or POX1 sequences in addition to the Δ12 desaturase expression cassette.

These constructs are introduced into the cells of a strain of Y. lipolytica having the fad2KO1 mutation in the endogenous Δ12 desaturase, or into strain INVSc1 or D5A of S. cerevisiae. Cells which have the genetic construct inserted into the POX2 or POX1 gene are identified and selected and the sequence confirmed by PCR with flanking and internal oligos. The transformants are grown in YPD medium and in a high glucose/low nitrogen medium in either the presence or the absence of oleic acid in the medium, to test the efficiency of each Δ12 desaturase under different growth conditions. The total fatty acid (TFA) composition of the lipid in the cells is determined, as well as for the TAG and polar lipid fractions of extracted lipid from the cells. The Δ12 desaturase efficiency is calculated by the formula (% LA and products derived from LA)×100/(% oleic acid+% LA and products derived from LA).

To test the efficiency of the conversion of LA to DGLA for the synthesis of ω6 fatty acids in yeast cells or other microbes, several genetic constructs were designed as follows.
Construct E: for production of DGLA by the Δ6 desaturase pathway. Encoding O. tauri Δ6 desaturase and P. cordata Δ6 elongase.
Construct F: for production of DGLA by the Δ9 elongase pathway. Encoding P. pinguis Δ9 elongase and P. salina Δ8 desaturase.
Construct G: for production of DGLA by both pathways. Encoding O. tauri Δ6 desaturase, P. cordata Δ6 elongase, P. pinguis Δ9 elongase and P. salina Δ8 desaturase.
   Each of these constructs are made with linked selectable marker gene and introduced into Y. lipolytica or S. cerevisiae. The transformants are grown in YPD medium and in a high glucose/low nitrogen medium in either the presence or the absence of LA added to the medium, to test the efficiency of each gene combination under different growth conditions. The total fatty acid (TFA) composition of the lipid in the cells is determined, as well as for the TAG and polar lipid fractions of extracted lipid from the cells. The conversion efficiency of LA to DGLA and derived products is calculated for TFA and each fraction by the formula (% DGLA and products derived from DGLA)×100/(% LA+% products derived from LA including DGLA).

A further series of constructs was designed corresponding to the second series E-G but adding a gene encoding a Δ12 desaturase in each case, selected from constructs A1-D2. The constructs are introduced into Y. lipolytica or S. cerevisiae and the fatty acid composition of cells grown in various media determined. The conversion efficiency of oleic acid to DGLA is calculated by the formula (% DGLA and products derived from DGLA)×100/(% oleic acid+% LA and products derived from LA).

In the experiments described above, each of the protein coding regions is codon optimised for increased expression in the appropriate yeast cells, the nucleotides immediately 5′ of the ATG start codon are optimised for translation, and promoters are optimised for efficiency of expression. Where two, three or more different genes are expressed, different promoters and transcription termination/polyadenylation regions are used for each gene in order to minimise the possibility of re-arrangements or gene deletions occurring during the cloning or transformation steps. GoldenGate assembly methods such as described in Example 1 may be used for multi-gene constructs.

Additional constructs are designed for producing other ω6 fatty acids in the microbes:
Construct H1, for production of ARA. Encoding O. tauri Δ6 desaturase, P. cordata Δ6 elongase, P. salina Δ5 desaturase and a Δ12 desaturase selected from constructs A1-D2.
Construct H2, for production of ARA. Encoding O. tauri Δ6 desaturase, P. cordata Δ6 elongase, M. alpina Δ5 desaturase and a Δ12 desaturase selected from constructs A1-D2.
Construct I, for production of DTA. Encoding O. tauri Δ6 desaturase, P. cordata Δ6 elongase, P. salina Δ5 desaturase, P. cordata Δ5 elongase and a Δ12 desaturase selected from constructs A1-D2.
Construct J1, for production of DPA-ω6. Encoding O. tauri Δ6 desaturase, P. cordata Δ6 elongase, P. salina Δ5 desaturase, P. cordata Δ5 elongase, P. salina Δ4 desaturase and a Δ12 desaturase selected from constructs A1-D2.
Construct J2, for production of DPA-ω6. Encoding O. tauri Δ6 desaturase, P. cordata Δ6 elongase, P. salina Δ5 desaturase, P. cordata Δ5 elongase, Thraustochytrium Δ4 desaturase and a Δ12 desaturase selected from constructs A1-D2.
Construct L, for production of GLA. Encoding O. tauri Δ6 desaturase and a Δ12 desaturase selected from constructs A1-D2.
Construct M, for production of GLA. Encoding M. alpina Δ6 desaturase and a Δ12 desaturase selected from constructs A1-D2.
Construct N, for production of GLA. Encoding O. tauri Δ6 desaturase and M. alpina Δ6 desaturase and a Δ12 desaturase selected from constructs A1-D2.

These constructs are also introduced into mutants of Y. lipolytica and S. cerevisiae which have been modified to reduce the synthesis and/or accumulation of TAG relative to polar lipids, in particular dga1, dga2 and lro1 mutants, or to modify the ratio of the different phospholipid classes, in particular the ratio of PE to PC.

### Example 17. Modification of Microbes to Reduce Fatty Acid Catabolism

Y. lipolytica is considered to be an oleaginous yeast since it can produce more than 20% by weight of lipid (dry cell weight), in some strains up to at least 77% total fatty acid content under growth conditions with limited nitrogen (Friedlander et al., 2016). The degradation and remobilization of lipids is driven by the β-oxidation pathway, which occurs in the peroxisome of microbes such as Y. lipolytica. Through this pathway, acyl-CoAs are catabolised via the activity of an acyl-CoA oxidase and the acyl chains are eventually broken down into acetyl-CoA molecules which are released from the peroxisome. Peroxisomal fatty acid β-oxidation is initiated by the activity of acyl-CoA oxidases, encoded by a single POX1 gene in S. cerevisiae and by six different POX genes, POX1 to POX6, in Y. lipolytica. The inventors considered that, by limiting the degradation of acyl-CoAs, the acyl chains could be utilised for the production and accumulation of polar lipids.

Phospholipids are also subject to degradation and the remobilization of lipids. The hydrolysis of fatty acyl groups from phospholipids, from the sn-1 and sn-2 position, is mediated through the activity of phospholipase B (PLB). The fate of the resulting free fatty acids is either degradation via the peroxisomal fatty acid β-oxidation pathway or recycled into the fatty acid synthesis pathway for further elongation and incorporation into lipids. The inventors considered that, by limiting the recycling of acyl chains from phospholipids, that the total production and accumulation of polar lipids could continue with reduced regulation.

Experiments were therefore designed to reduce phospholipid turnover through the inactivation of one or more genes encoding PLB1, the most active acyl-CoA oxidase genes, including POX1-3 and 5, and MFE1, and also to interfere with the biogenesis of peroxisomes through the inactivation of the PEX10 gene in Y. lipolytica.

Genetic Constructs for Introducing a POX1 Gene Deletion into Y. lipolytica

To delete the protein coding sequence of the POX1 gene and other genes involved in β-oxidation of fatty acids from the Y. lipolytica genome, thereby providing null mutations, the general strategy is followed as described in Example 11 (FIG. 6). As before, the genetic cassette for introducing the gene deletions had the protein coding region of the gene of interest replaced with a selectable marker gene, flanked by 5′ upstream and 3′ downstream sequences of 1,000 basepairs which provided for integration of the genetic cassette by recombination into the endogenous gene. The primers used in the amplifications of the selectable marker genes had AsiSI restriction enzyme sites rather than SacII sites.

The nucleotide sequence of the POX1 gene of Y. lipolytica and its upstream and downstream sequences were extracted from the KEGG Yarrowia database (www.genome.jp/kegg-bin/show_organism?org-yli) using the published YARLI gene identifier, as gene YALI0E32835g, nucleotides 3897102 to 3899135 of chromosome E, Accession No. CR382131.1. The nucleotide sequence of the POX1 gene is provided herein as SEQ ID NO:87 including 1,000 nucleotides upstream of the protein coding sequence followed by the protein coding sequence and 1,000 nucleotides downstream of the protein coding sequence.

The amino acid sequence of the encoded POX1 polypeptide is provided as SEQ ID NO: 88. Y. lipolytica POX1 is a protein of 677 amino acid residues.

The 5′ upstream and 3′ downstream regions adjacent to the POX1 protein coding region were amplified from genomic DNA from Y. lipolytica strain W29 (FIG. 6). Each amplification reaction used Taq DNA Polymerase with ThermoPol Buffer and a pair of oligonucleotide primers. By this means, the 5′ upstream fragment was adapted by adding restriction enzyme sites for AscI at its 5′ end and AsiSI at its 3′ end. Similarly, the 3′ downstream fragment was adapted by adding restriction enzyme sites for AsiSI at its 5′ end and NotI at its 3′ end. The amplified DNA fragments were digested with AsiSI and ligated with T4 DNA Ligase using standard protocols and inserted into vector into the vector pCR Zero Blunt TOPO. The nucleotide sequence of the cloned insert is confirmed.

The DNAs of pAT123 including the hygromycin resistance gene and pAT124 including the nourseothricin resistance gene (Example 9) were digested with AsiSI and the fragments spanning the genes purified using a gel extraction kit (Qiagen, USA). The DNA fragments are separately ligated with the amplified 5′ and 3′ regions which are digested with AsiSI and treated with calf intestinal alkaline phosphatase. The ligation mixes are transformed into E. coli DH5α competent cells. DNA is prepared from at least five colonies for each ligation and DNA samples from the colonies are screened by digestion with restriction enzymes and agarose gel electrophoresis to identify and confirm that the correct insertions had occurred between the 5′ upstream and 3′ downstream sequences. The resultant constructs having the Hyg or Natl antibiotic resistance gene sequences flanked by the 5′ upstream and 3′ downstream sequences from POX1 are selected and retained.

Introduction of POX1 Deletion Constructs into Y. lipolytica

To introduce the genetic construct containing the hygromycin resistance gene replacing the POX1 protein coding region into Y. lipolytica and identify genetically modified Δpox1 cells from the transformation, the transformation protocol described in Example 9 is followed. Transformed cells are selected on YPD plates containing 250 μg/mL hygromycin. Antibiotic resistant colonies are screened by PCR for the POX1 gene insertion. One of the transformed cell lines is selected and retained as a Y. lipolytica pox2 deletion mutant and designated strain pox1KO1.

The strain pox1KO1 is compared to its corresponding wild-type strain by growth in YPD, a rich medium, and a high glucose/low nitrogen medium that induces TAG synthesis, to determine the increase in polar lipid or TAG accumulation. The amount of polar lipid and the incorporation of ω6 fatty acids into polar lipids is also assessed by culturing the mutant strain in media containing one or more of the ω6 fatty acids, or by introduction of a genetic construct for production of the ω6 fatty acids (Example 16).

Genetic Constructs for Introducing a POX2 Gene Deletion into Y. lipolytica

To delete the protein coding sequence of the POX2 gene from the Y. lipolytica genome, the same strategy is used as for the POX1 deletion. The nucleotide sequence of the POX2 gene of Y. lipolytica and its upstream and downstream sequences were extracted from the KEGG Yarrowia database (www.genome.jp/kegg-bin/show_organism?org-yli), using the published YARLI gene identifier, as gene YALI0F10857g, nucleotides 1449289 to 1451391 of chromosome F, Accession No. CR382132.1. The nucleotide sequence of the POX2 gene is provided as SEQ ID NO:77 including 1,000 nucleotides upstream and downstream of the protein coding sequence. The amino acid sequence of the encoded POX2 polypeptide is provided as SEQ ID NO:78. Y. lipolytica POX2 is a protein of 700 amino acid residues.

Genetic Constructs for Introducing Other Targeted Gene Deletions into Y. lipolytica

To delete the protein coding sequence of MFE1 (SEQ ID NO:91), PEX10 (SEQ ID NO: 93), PLB1 (SEQ ID NO:95), SNF1 (SEQ ID NO:97), and other genes of interest from the Y. lipolytica genome, the same strategy is used as for the deletion of POX1, described above. The nucleotide sequences, including 1,000 nucleotides upstream and downstream of the protein coding sequence, and the encoded polypeptide sequences of the target genes have been provided (SEQ ID NOs: 91, 93, 95, and 97), which were extracted from the KEGG Yarrowia database (www.genome.jp/kegg-bin/show_organism?org-yli), using the published YARLI gene identifier.

### Example 18. Modification of Microbes to Increase Fatty Acid Synthesis

As described in Example 17, single gene mutants are produced in Y. lipolytica that have deletions in OPI1 or SPO14, that enable a greater flux of fatty acids for lipid accumulation.

### Example 19. Lipid Fractionation

Crude lipid preparations may be fractionated with organic solvents to provide purer polar lipids or fractions having mostly neutral (non-polar) lipids including TAG (e.g. U.S. Pat. No. 7,550,616). For example, some reported methods use differential solubility of neutral and polar lipids in organic solvents such as ethanol or acetone. To test some of these methods, fractionation of several lipids having a mixture of substantial neutral and polar lipids was attempted, including egg yolk lipid and krill lipid, as model systems.

The lipids in chicken eggs are present mostly in the yolk fraction which constitutes about 33% lipid by weight. The lipids, which are closely associated with proteins in the yolk, are mostly TAG (66% by weight), with phospholipids (PL, 28%) and cholesterol and its esters (6%) present in lower amounts (Belitz et al., 2009). The PL contains some ω3 and ω6 fatty acids (Gladkowski et al., 2011). Based on the method of Palacios and Wang (2005), Gladkowski et al. (2012) extracted PL from egg yolk with ethanol and then purified the PL by removing neutral lipids by precipitation of the PL with cold acetone.

Fresh egg yolk (17 g), egg lecithin powder (20.4 g; Lesen Bio-Technology Co, Xi′an, China) and krill oil from Euphausia superba (17.7 g) obtained from commercially available krill oil capsules (Bioglan Red Krill Oil; Natural Bio Pty Ltd, Warriewood, NSW, Australia) were each mixed with 60 ml of ethanol and stirred for 30 min. The ethanol supernatant was collected after centrifuging the mixture. The precipitate was extracted twice more, each time with 60 ml ethanol. The extraction mixtures were centrifuged and the ethanol supernatants combined. Each precipitate was retained for extraction of neutral lipids. The ethanol from the combined supernatants was evaporated using a SR-100 rotary evaporator (Buchi, Switzerland) operating at 400 rpm with a vacuum of 15 mbar, with the chiller set at −16° C. and the waterbath at 37° C. This yielded 3.2 g of PL-enriched lipid extract from the 17 g of fresh egg yolk, 5.86 g from the 20.4 g of egg lecithin powder and 17.83g of enriched PL recovered from the krill oil. The lipid recovered from the krill oil probably still contained a small amount of solvent. Nevertheless, the recovery of essentially 100% indicated that the krill oil from the capsules was highly enriched for PL to begin with.

Aliquots of the recovered lipids were analysed by TLC as described in Example 1 using hexane: diethylether: acetic acid (70:30:1; v/v/v) as solvent. The ethanol extracts from fresh egg yolk and egg yolk lecithin powder were observed to contain substantial amounts of polar lipid as well as a small amount TAG, while the krill oil extract had no detected TAG.

To further purify the polar lipids from the fresh egg yolk, the dried extract was dissolved in 30 ml of hexane and the solution cooled in an ice bath to 0° C. Next, 60 ml of cold acetone (−20° C.) was gradually added to the solution and the mixture kept cold for at least 20 min to precipitate the PL. Other experiments showed that more precipitate formed by keeping the mixtures at 0° C. overnight. The precipitate was collected and dried under vacuum. Samples of the lipid were dissolved in chloroform and analysed by TLC to estimate the polar lipid and TAG contents. The acetone precipitate was shown to have mostly polar lipid with some TAG. To further purify the polar lipid, the precipitate was washed 5 times with 20 ml portions of cold acetone (−20° C.) to remove more of the TAG and other neutral lipids such as cholesterol. The residual solvent was removed from the washed precipitate by rotary evaporation at room temperature for 10 h. The lipid yield was measured gravimetrically and a small aliquot used for analysis of the fatty acid composition by GC quantitation of FAME. From the initial input of 17 g of fresh egg yolk, 1.1 gram of purified polar lipid was recovered. An aliquot of this extracted lipid was analysed by TLC and was observed to be essentially devoid of any neutral lipids, including TAG. These observations were consistent with those reported by Gladkowski et al. (2012) who found their extracts to be 96% pure PL.

Neutral lipid was extracted from the precipitates after the ethanol extraction of the egg yolk and egg yolk powder by extracting the precipitate twice with 50 ml of hexane. The combined hexane solution containing the neutral lipid was washed four times, each time with 50 ml of 90% ethanol. The hexane was then evaporated under reduced pressure to provide the purified neutral lipids from egg yolk.

To determine the fatty acid composition of the extracted lipids, the total fatty acids in aliquots were converted to FAME for GC analysis as described in Example 1. This included the samples (1^{st} ppt) after the ethanol extraction but before the hexane/acetone precipitation, as well as samples (2^{nd} ppt) after the hexane/acetone precipitation. The data are shown in Table 45. The ethanol-soluble lipid isolated from the fresh egg yolk and acetone precipitated lipid purified therefrom contained C16:0 and C18:0 as the main saturated fatty acids. The first lipid precipitate from fresh egg yolk containing 24.7% (C16:0) and 15.6% (C18:0) while the more purified polar lipid contained 27% (C16:0) and 16% (C18:0). The amount of LA in the 2^{nd} precipitate was slightly higher than in the 1^{st} precipitate; LA is present at greater amounts in PL than in TAG. Both fresh egg yolk and the purer polar lipid preparations also contained ω6 and ω3 LC-PUFA. For instance, the fresh egg yolk 1^{st} precipitate contained 5.3% C20:4 (ARA), 2.3% C20:5 (EPA) and 5% C22:6 (DHA) while more purified polar lipid preparation contained 5.3% ARA and 4% DHA. The first precipitate from the krill oil and the more purified polar lipid from the krill oil had C16:0 as their main saturated fatty acid. The krill oil 1^{st} precipitate and the more purified polar lipid also contained substantial amounts of ω3 LC-PUFA, namely 1.1% ARA, 34.7% EPA and 19.0% DHA in the 1^{st} precipitate, while the more purified polar lipid contained 1.1% ARA, 48.1% EPA and 25.7% DHA. The precipitated lipid from the egg yolk lecithin powder had 17% C16:0 and 4% C18:0 but was low in the LC-PUFA EPA and DHA. It was considered that the low LC-PUFA content of the lecithin powder was likely due to oxidative breakdown of those polyunsaturated fatty acids during its production or storage.

An alternative method to purify polar lipids by fractionation from a total lipid preparation is to use silica-based column chromatography such as, for example, use of SPE columns (HyperSep aminopropyl, ThermoFisher, UK).

TABLE 45
Fatty acid composition of polar lipids purified from egg yolk and krill oil capsules.

| Polar lipid | | | | | | | | | | |
|---|---|---|---|---|---|---|---|---|---|---|
| fractions | C12:0 | C14:0 | C15:0 | C15:1 | C16:0 | C16:1 | C18:0 | C18:1 | C18:1A11 | C18:2(LA) |
| Egg yolk 1^{st} | 0.0 | 0.6 | 0.1 | 0.2 | 24.7 | 2.2 | 15.6 | 26.9 | 0.0 | 14.8 |
| ppt | | | | | | | | | | |
| Egg yolk 2^{nd} | 0.0 | 0.2 | 0.1 | 0.2 | 27.1 | 1.0 | 16.5 | 23.6 | 0.0 | 20.2 |
| ppt | | | | | | | | | | |
| Krill oil 1^{st} | 0.0 | 3.1 | 0.3 | 0.1 | 25.7 | 1.9 | 1.1 | 6.1 | 0.0 | 2.6 |
| ppt | | | | | | | | | | |
| Krill oil 2^{nd} | 0.0 | 4.0 | 0.4 | 0.1 | ? | 0.2 | 1.1 | 8.3 | 0.1 | 3.0 |
| ppt | | | | | | | | | | |

Fatty acid composition of polar lipids purified from egg yolk and krill oil capsules.

| | | | | | | C20: | C20: | | | |
|---|---|---|---|---|---|---|---|---|---|---|
| | C18:3 | C19:0 | C20:0 | C20:2 | C20:3 | 4ARA | 5EPA | C22:2 | C22:6DHA | C24:0 |
| Egg yolk 1^{st} | 0.5 | 0.2 | 0.0 | 0.3 | 0.3 | 5.3 | 2.3 | 0.0 | 5.3 | 0.4 |
| ppt | | | | | | | | | | |
| Egg yolk 2^{nd} | 0.4 | 0.0 | 0.0 | 0.4 | 0.3 | 5.3 | ? | 0.0 | 4.2 | 0.1 |
| ppt | | | | | | | | | | |
| Krill oil 1^{st} | 1.9 | 0.0 | 0.0 | 0.2 | 0.2 | 0.8 | 34.7 | 1.3 | 19.0 | 0.6 |
| ppt | | | | | | | | | | |
| Krill oil 2^{nd} | 2.6 | 0.0 | 0.0 | 0.2 | 0.2 | 1.1 | 48.1 | 1.7 | 25.7 | 0.9 |
| ppt | | | | | | | | | | |

### Example 20. Maillard Reactions

The Maillard reaction is a chemical reaction between a reducing sugar and an amino group, for example in a free amino acid, with application of heat. Like caramelisation, it is a form of non-enzymatic browning. In this reaction, the amino group reacts with a carbonyl group of the sugar and produces N-substituted glycosylamine and water. The unstable glycosylamine undergoes an Amadori rearrangement reaction and produces ketosamines. The ketosamines can react further in different ways to produce reductones, diacetyl, aspirin, pyruvaldehyde, and other short-chain hydrolytic fission products. Finally, a furan derivate may be obtained which reacts with other components to polymerize into a dark-coloured insoluble material containing nitrogen.

The outcome of the Maillard reaction depends on temperature, time and pH. For example, the reaction slows at low temperature, low pH and low water activity (Aw) levels. The browning colour occurs more quickly in alkaline conditions because the amino group remains in the basic form. The reaction peaks at intermediate water activities such as Aw of 0.6-0.7. In addition to colour, many volatile aroma compounds are typically formed during the Maillard reaction. Flavour-intensive compounds may be formed in the presence of the sulphur-containing amino acids methionine or cysteine or other sulphur containing compounds such as thiamine. Unsaturated fatty acids and aldehydes formed from fatty acids also contribute to the formation of heterocyclic flavour compounds during the Maillard reaction (Feiner, 2006). In view of this contribution of unsaturated fatty acids to formation of flavours and aromas, the inventors tested the extracted egg yolk polar lipid preparation from Example 19 as a model system for Maillard reactions.

In an initial experiment, 26 mixtures for Maillard reactions were assembled containing a matrix of components in a base medium and either containing 15 mg of the extracted egg yolk polar lipid (Example 2) or lacking the lipid (controls). The reactions were carried out in 2 ml volumes in 20 ml glass vials with tightly sealing screw top lids. To deposit a precise amount of the extracted lipid into the vials, the lipid was dissolved in hexane at a concentration of 1 mg/μl of the solvent. An aliquot of 50 μL of the lipid solution containing 50 mg enriched polar lipid was pipetted into the vials for reactions having the lipid. The hexane was then evaporated under a nitrogen flow. The other components in each mixture were added to the vials in the following order. Components were added to provide final concentrations of 10 mM xylose as the sugar, 0.1 mM thiamine hydrochloride, and either 5 mM cysteine or 5 mM cystine as a sulphur-containing amino acid. These components were dissolved in a final concentration of 32.6 mM potassium phosphate buffer pH 6.0 or 5.3, prepared from potassium dihydrogen phosphate and dipotassium hydrogen phosphate. Some mixtures also included one or more of 15 mg/mL yeast extract, 3.5 mg/L iron (Fe²⁺) in the form of iron fumarate (Apohealth, NSW, Australia) and 2 mM L-glutamic acid monosodium salt hydrate. The presence or absence of yeast extract was intended to test whether it would either mask, or enhance, the aroma produced from the extracted lipid having PL, or have no effect.

The assembled mixtures were sonicated for 30 min and then heated for 15 min in an oven set at 146° C. During the heat treatment, the vials were tightly sealed. The vials were cooled until warm to the touch about 15 min later, and then opened briefly for sniffing by a panel of 4 volunteers (P1 to P4). These included 2 males and 2 females, ages ranging from 24-65 years. The volunteers did not know the composition of any of the vials prior to sniffing the contents and the vials were sniffed in a random order as selected by the volunteers. The volunteers sniffed coffee beans between sniffing each test sample to reset their olefactory senses. Their descriptions of the aromas were recorded without any comments being shared until the sniffing was completed.

The four participants varied considerably in their descriptions of the detected aromas of the 26 mixtures. Despite these variations, the reaction mixtures containing the added polar lipid preparation were generally recognized as having a more meaty/meat-like aroma compared to the control samples lacking the polar lipid, confirming the role of the lipids in contributing to a meaty aroma following the heating-induced Maillard reactions. Samples containing the yeast extract, the iron fumarate, or both, were identified as having more meat-like or meaty related aromas, described as beef or chicken by 3 of the 4 participants. Therefore, the base composition with those components was selected for further investigation.

Several further experiments were carried out to test variations of the Maillard reaction mixtures in terms of the composition of the base medium. In one experiment, the xylose was substituted with either glucose or ribose as the sugar component. In another experiment, Fenugreek (Trigonella foenum-graecum) leaf power was added to some of the mixtures at 10 mg per 1 ml reaction. Fenugreek leaf powder was tested as this herb has long been used in food cooking to enhance the flavour of dishes such as in curries or in combination with other herbs or spices such as cumin and coriander. Some reaction mixtures contained 30 mg of a yeast extract powder whereas others did not. Control reactions had the same base media compositions but lacked the extracted polar lipid preparation. The reaction mixes were sonicated as a batch by placing the vials in a floating foam and placed in a sonicator (Soniclean, Thermoline) set up at a medium power for 30 min and then heat treated in an oven at 140° C. for about 60 min. The vials containing the reaction mixtures were cooled slowly over about 15 min until warm to the touch. The vials were opened briefly by each of 10 volunteers and the contents sniffed, and their descriptions of the aromas recorded. The volunteers ranging in age from 29 to 65 years and were from a range of ethnic backgrounds. The reactions had been coded with random 3-digit numbers to avoid bias, and the volunteers sniffed coffee beans between vials, as before.

The recorded responses to the sniffing of the reaction mixtures were generally consistent with those of the previous experiment. Most of the mixtures containing the extracted lipid elicited favourable comments, in particular the ones containing ribose rather than glucose for meaty aromas. The use of yeast extract could enhance the meaty aroma but was not considered to generate species-specific aromas e.g. a beef aroma versus a chicken aroma. The addition of the herbal powder, Fenugreek, to the mixtures increased the sensation of a soupy or vegetable aroma with a pleasant vegetable note. It was concluded that a variety of medium compositions and components could be used with the extracted lipid, with ribose preferred over glucose as the sugar component.

### Example 21. Isolation of Mortierella and Mucor Strains from Soil Samples

Mortierella alpina is a filamentous and saprophytic fungus of the family Zygomycete which is commonly found to inhabit soils from temperate grasslands (Botha et al., 1998). Some strains of this species are used commercially to produce oils containing polyunsaturated fatty acids (PUFA), specifically the ω6 fatty acids arachidonic acid (C20:4; ARA), linoleic acid (C18:2; LA) and γ-linolenic acid (C18:3; GLA) (Ho and Chen, 2008). Another fungal species, Mucor hiemalis is a zygosporic fungus of the Order Mucorales that is ubiquitous in nature and can be found, for example, in unspoiled foods. It has also been used industrially as a biotransforming agent of pharmacological and chemical compounds, as well as being a potential source of ω6 fatty acids. The present inventors therefore sought to isolate strains of Mortierella alpina, Mucor hiemalis and related species from soil samples obtained from some temperate regions of Australia.

The Biomes of Australian Soil Environments (BASE) project database is a database that contains integrated information about microbial diversity and function for microbial isolates from more than 1,400 soil samples taken from 902 locations across Australia (Bisset et al., 2016). It includes associated metadata for all of the soil samples across extensive environmental gradients, including information from phylogenetic marker sequencing of bacterial 16S rRNA, archaeal 16S rRNA and eukaryotic 18S rRNA genes to characterise the diversity of microbes in community assemblages. Fungal diversity was informed by the 18S rRNA gene amplicon sequences. However, because fungi are an important group of organisms of soils, and because the internal transcribed spacer (ITS) region is more informative than 18S rRNA for many fungal groups, ITS sequences were also included by sequencing fungal-specific ITS amplicons to characterise fungal community assemblages. These amplicons cover the diverse range of microbes resident in soils (Bisset et al., 2016).

The BASE database was therefore interrogated to identify soil samples from the BASE archive that might contain fungal species in the Mortierella or Mucor genera. The interrogation used a M. alpina strain ATCC 32222 internal transcribed spacer 1 (ITS; SEQ ID NO: 103) as a query. More than 12 soil samples were identified as candidates containing these strains from these genera. One such soil sample, designated 102.100.100/14183, was identified and retrieved from the archive for isolation of fungal strains. In addition, two other soil samples, designated Namadgi sample I and Namadgi sample II, were collected from an open grassland field from the temperate Namadgi region of the Australian Capital Territory, Australia. About 5-10 mg of fine soil from each sample was suspended in 3 ml of PBS and vortexed for 2 min. For each soil sample, 100 μl of soil suspension was spread on each of 10 plates of malt extract agar (MEA), containing 20 g/l malt extract and 20 g/l agar, and incubated at 4° C. in the dark (Botha et al., 1998). The plates were observed periodically for growth of fungal colonies. After 8-12 days, mycelia from the edge of distinct colonies were transferred through agar slices to fresh MEA plates and incubated at 4° C. until colonies were 1 to 4 cm in diameter. To further purify the colonies, mycelia from the edge of each colony were transferred through agar slices to fresh MEA plates and incubated at ambient temperatures for 4 days. Colonies that appeared pure through visual inspection were inoculated into 5 ml of malt extract broth and grown at ambient temperature in a static culture for 5 days. A total of 67 fungal strains were thereby isolated from the three soil samples.

Genomic DNA was isolated from each hyphal biomass using the YeaStar Genomic DNA kit (Zymo research, Catalog No. D2002). An internal transcribed spacer (ITS) was amplified through PCR as described by Ho and Chen (2008) using oligonucleotide primers xMaF1 GGAAGTAAAAGTCGTAACAAGG (SEQ ID NO: 147) and xMaF2 TCCCCGCTTATTGATATGC (SEQ ID NO: 148). The nucleotide sequence of the ITS from the amplicons from each isolate were determined by Sanger sequencing. The obtained sequences were compared to sequences within the NCBI repository using BLAST. The closest hits, with at least 95% nucleotide sequence identity for each isolate and often at 98% or 99% identity, were used to identify the species for each fungal isolate.

At least four different fungal species were identified based on the ITS homology, which correlated with the four distinctly different morphological features observed when the fungal colonies were grown on the MEA plates. Interestingly, three of the species were isolated mostly from one of the three soil samples but not the others: Mucor hiemalis was found predominantly in Namadji I soil, Mortierella alpina in soil from sample 102.100.100/14183 and isolates of presumed Mortierella sp. in the Namadji II soil. A single colony of Mortierella elongata was isolated from each of the Namadji I and II soil samples. The ITS sequences from the presumed Mortierella sp. isolates identified from the Namadji II soil sample were not found in the NCBI database at a 95% identity level as a minimum. Nevertheless, based on lower homology hits of the ITS sequences, these isolates were considered to most likely be of Mortierella sp. or a species closely related to the Mortierella genus. The nucleotide sequences for the ITS regions for 43 fungal isolates and the deduced species names are listed in Table 46. Selected isolates were designated as strains yNI0121 to yNI0131 and yNI0133 to yNI0135 (Table 46).

The ITS regions amplified with primers xMaF1 and xMaF2 produced amplicons having a length of between 639 and 647 basepairs for the Mucor hiemalis strains, between 668 and 672 basepairs for the Mortierella alpina strains, between 628 and 652 basepairs for the Mortierella sp. isolates, and between 640 and 659 for the two Mortierella elongata strains. The length of this ITS amplicon was therefore useful in helping to distinguish between the four species.

TABLE 46
Species identities of isolated soil fungi.

| | ITS | | |
|---|---|---|---|
| | sequence | | Desig- |
| | (SEQ | ITS highest | nated |
| Identifier | ID NO) | homology to | strain ID |
| 14183 isolate 1 | 104 | Mucor hiemalis | — |
| 14183 isolate 2 | 105 | M. alpina | yNI0133 |
| 14183 isolate 3 | 106 | M. alpina | yNI0134 |
| 14183 isolate 4 | 107 | M. alpina | yNI0135 |
| 14183 isolate 21 | 108 | M. alpina | — |
| 14183 isolate 22 | 109 | M. alpina | — |
| 14183 isolate 23 | 110 | M. alpina | — |
| 14183 isolate 24 | 111 | Possibly Trichoderma | — |
| | | asperellum | |
| 14183 isolate 25 | 112 | M. alpina | — |
| Namadji I isolate 1 | 113 | Mucor hiemalis | yNI0121 |
| Namadji I isolate 3 | 114 | Mucor hiemalis | yNI0122 |
| Namadji I isolate 4 | 115 | Mucor hiemalis | yNI0124 |
| Namadji I isolate 5 | 116 | Mucor hiemalis | yNI0123 |
| Namadji I isolate 6 | 117 | Mucor hiemalis | — |
| Namadji I isolate 8 | 118 | Mucor hiemalis | — |
| Namadji I isolate 9 | 119 | Mucor hiemalis | — |
| Namadji I isolate 10 | 120 | Mucor hiemalis | — |
| Namadji I isolate 11 | 121 | Mortierella elongata | yNI0125 |
| Namadji I isolate 12 | 122 | Mucor hiemalis | — |
| Namadji I isolate 14 | 123 | Mucor hiemalis | — |
| Namadji I isolate 15 | 124 | Mucor hiemalis | — |
| Namadji I isolate 21 | 125 | Mucor hiemalis | — |
| Namadji II isolate 1 | 126 | Mortierella sp. | yNI0126 |
| Namadji II isolate 2 | 127 | Mortierella sp. | yNI0127 |
| Namadji II isolate 3 | 128 | Mortierella sp. | yNI0128 |
| Namadji II isolate 4 | 129 | Mortierella sp. | yNI0129 |
| Namadji II isolate 5 | 130 | Mortierella sp. | yNI0130 |
| Namadji II isolate 6 | 131 | Mortierella sp. | — |
| Namadji II isolate 7 | 132 | Mortierella sp. | — |
| Namadji II isolate 8 | 133 | Mortierella sp. | — |
| Namadji II isolate 9 | 134 | Mortierella elongata | yNI0131 |
| Namadji II isolate 10 | 135 | Mortierella sp. | — |
| Namadji II isolate 11 | 136 | Mortierella sp. | — |
| Namadji II isolate 12 | 137 | Mortierella sp. | — |
| Namadji II isolate 13 | 138 | Mortierella sp. | — |
| Namadji II isolate 14 | 139 | Mortierella sp. | — |
| Namadji II isolate 15 | 140 | Mortierella sp. | — |
| Namadji II isolate 16 | 141 | Mortierella sp. | — |
| Namadji II isolate 17 | 142 | Mortierella sp. | — |
| Namadji II isolate 18 | 143 | Mortierella sp. | — |
| Namadji II isolate 19 | 144 | Mortierella sp. | — |
| Namadji II isolate 20 | 145 | Mortierella sp. | — |
| Namadji II isolate 21 | 146 | Mortierella sp. | — |

### Fatty Acid Composition and Oil Content of Fungal Isolates

For analysis of lipid in these fungal isolates, agar slices from the edges of colonies were placed on fresh SD agar plates and allowed to grow for 4-6 days at ambient temperature, until the colonies exceeded 3 cm in diameter. SD medium was used in this experiment as it does not have yeast extract which may have some lipid that might contaminate the fungal biomass. Hyphal biomass was harvested from the plates and suspended in sterile water for pelleting. After washing the hyphal biomass with ethanol, lipid was extracted using a chloroform/methanol solvent (Bligh and Dyer, 1959) and fractionated on TLC plates to obtain TAG and polar lipid fractions. The fatty acid composition of the TAG and polar lipid fractions were determined by GC analysis of FAME as described in Example 1.

The data for strains yNI0121 to yNI0131 and yNI0133 to yNI0135 are presented in Table 47. The fatty acid compositions showed distinct differences between the four species, but with some similarities as well. All four species produced polyunsaturated ω6 fatty acids having 18 or 20 carbons and 3 or 4 desaturations in the acyl chains, namely GLA alone of the ω6 fatty acids in the case of Mucor hiemalis, or all three of GLA, DGLA and ARA for all of the Mortierella isolates. Nearly all of the isolates produced at least 20% such PUFA (sum of GLA, DGLA and ARA) in the polar lipid fraction, up to about 38%, as a percentage by weight of the total fatty acid content in those fractions. The Mucor hiemalis strains yNI0121 to yNI0124 all produced GLA at about 10% in the total fatty acid content of the TAG and between 26% and 30% GLA in the polar lipids. It was concluded that these Mucor strains preferentially accumulated the ω6 PUFA in their polar lipids. These strains did not produce ARA or DGLA levels at detectable levels, or only at trace amounts in the polar lipid fractions, indicating that they did not have the ability to elongate GLA to DGLA i.e. they lacked a fatty acid Δ6 elongase. This is consistent with published reports for Mucor strains (Certik et al., 1993). LA (C18:206) was the most abundant fatty acid in the TAG fraction of the Mucor strains, but not in any of the three Mortierella species. These strains produced 12-18% TAG and a relatively high amount of polar lipid, up to about 7% by dry weight, under the growth conditions on SD agar plates used to culture the strains for this analysis. Considering that the extraction and recovery of lipid fractions from the process including TLC fractionation would have been less than 100%, the total lipid content of these Mucor hiemalis strains was greater than 20% and therefore these strains are oleaginous.

In contrast to the Mucor hiemalis strains, the Mortierella alpina strains yNI0133 to yNI0135 produced abundant ARA as well as GLA and DGLA. The ARA level in both the TAG and polar lipids was about 30% by weight of the total fatty acid content in those fractions. These M. alpina strains therefore did not exhibit any preference for accumulating the ω6 PUFA in polar lipid relative to TAG. The GLA and DGLA levels were about 2% and about 6%, respectively, in TAG, and about 4-7% and about 2-4%, respectively, in the polar lipid. Compared to the ARA levels, this indicated that the M. alpina strains have efficient Δ6 elongase and Δ5 desaturase enzymes. Genes encoding such enzymes have been isolated from other strains of M. alpina (Huang et al., 1999; Knutzon et al., 1998). The Mortierella alpina strains also produced about 4-5% of C24:0 in the TAG fractions

TABLE 47
Fatty acid composition of TAG and polar lipids from Mortierella and Mucor isolates from soil samples

| | | | | | | | | | | | C18: |
|---|---|---|---|---|---|---|---|---|---|---|---|
| Strain | | | | | C16: | C16: | | | C18: | C18: | 2Δ9, |
| ID | Species | C14:0 | C15:0 | C16:0 | 1Δ9 | 1Δ7 | C17:1? | C18:0 | 1Δ9 | 1Δ11 | 12 |
| | | TAG fatty acid composition | | | | | | | | | |
| yNI01 | Mucor | 1.4 | 0.1 | 17.4 | 0.5 | 0.2 | 0 | 10.8 | 37.9 | 0.1 | 18.3 |
| 21 | hiemalis | | | | | | | | | | |
| yNI01 | Mucor | 2.3 | 0.2 | 22.9 | 1.3 | 0.1 | 0 | 8.0 | 39.2 | 0.3 | 13.2 |
| 22 | hiemalis | | | | | | | | | | |
| yNI01 | Mucor | 2.6 | 0.2 | 24.8 | 0.8 | 0.1 | 0 | 5.4 | 37.0 | 0.1 | 15.5 |
| 23 | hiemalis | | | | | | | | | | |
| yNI01 | Mucor | 2.3 | 0.2 | 23.9 | 0.9 | 0.2 | 0 | 4.9 | 36.7 | 0.2 | 16.3 |
| 24 | hiemalis | | | | | | | | | | |
| yNI01 | Mortierella | 3.0 | 0.2 | 27.6 | 0.6 | 0.1 | 0.1 | 9.4 | 38.9 | 0.5 | 4.1 |
| 25 | elongata | | | | | | | | | | |
| yNI01 | Mortierella | 1.5 | 0.3 | 19.1 | 0.4 | 0.1 | 0.1 | 5.1 | 18.8 | 0.8 | 5.8 |
| 33 | alpina | | | | | | | | | | |
| yNI01 | Mortierella | 2.3 | 0.3 | 19.1 | 0.4 | 0.1 | 0.1 | 6.7 | 20.9 | 0.5 | 5.6 |
| 34 | alpina | | | | | | | | | | |
| yNI01 | Mortierella | 1.6 | 0.3 | 20.4 | 0.4 | 0.1 | 0.1 | 5.1 | 21.0 | 0.9 | 6.2 |
| 35 | alpina | | | | | | | | | | |
| yNI01 | Mortierella sp | 2.9 | 0.2 | 31.1 | 0.6 | 0.1 | 0.1 | 6.3 | 44.3 | 0.6 | 3.2 |
| 26 | | | | | | | | | | | |
| yNI01 | Mortierella sp | 3.5 | 0.2 | 28.3 | 1.1 | 0.2 | 0.1 | 3.5 | 42.7 | 0.9 | 4.0 |
| 27 | | | | | | | | | | | |
| yNI01 | Mortierella sp | 3.2 | 0.2 | 29.9 | 1 | 0.2 | 0.1 | 4.2 | 43.3 | 0.9 | 4.0 |
| 28 | | | | | | | | | | | |
| yNI01 | Mortierella sp | 3.4 | 0.2 | 28.4 | 1.1 | 0.2 | 0.1 | 3.1 | 42.0 | 1.0 | 4.1 |
| 29 | | | | | | | | | | | |
| yNI01 | Mortierella sp | 3.5 | 0.2 | 25.7 | 1.0 | 0.2 | 0.1 | 3.6 | 46.6 | 0.7 | 4.5 |
| 30 | | | | | | | | | | | |
| yNI01 | Mortierella | 2.1 | 0.3 | 19.1 | 0.3 | 0.2 | 0.1 | 10.6 | 38.9 | 0.2 | 4.2 |
| 31 | elongata | | | | | | | | | | |
| | | Polar lipid fatty acid composition | | | | | | | | | |
| yNI01 | Mucor | 0.7 | 0.2 | 13.8 | 0.4 | 0.2 | 0.1 | 2.5 | 20.4 | 0.1 | 32.4 |
| 21 | hiemalis | | | | | | | | | | |
| yNI01 | Mucor | 1.1 | 0.3 | 13.6 | 1.2 | 0.1 | 0 | 1.9 | 14.9 | 0.2 | 33.1 |
| 22 | hiemalis | | | | | | | | | | |
| yNI01 | Mucor | 1.1 | 0.3 | 15.3 | 0.6 | 0.2 | 0 | 1.8 | 15.6 | 0.1 | 32.3 |
| 23 | hiemalis | | | | | | | | | | |
| yNI01 | Mucor | 1.1 | 0.4 | 16.9 | 0.8 | 0.3 | 0.1 | 1.9 | 18.5 | 0.1 | 29.2 |
| 24 | hiemalis | | | | | | | | | | |
| yNI01 | Mortierella | 1.1 | 0.2 | 15.3 | 0.5 | 0.1 | 0.1 | 4.7 | 30.3 | 0.9 | 11.5 |
| 25 | elongata | | | | | | | | | | |
| yNI01 | Mortierella | 0.5 | 0.5 | 13.8 | 0.3 | 0.1 | 0.1 | 3.7 | 18.4 | 2.7 | 16.7 |
| 33 | alpina | | | | | | | | | | |
| yNI01 | Mortierella | 0.8 | 0.4 | 11.8 | 0.3 | 0 | 0 | 3.2 | 26.3 | 1.5 | 12.0 |
| 34 | alpina | | | | | | | | | | |
| yNI01 | Mortierella | 0.5 | 0.5 | 13.8 | 0.4 | 0.1 | 0.1 | 3.1 | 19.5 | 3.0 | 18.1 |
| 35 | alpina | | | | | | | | | | |
| yNI01 | Mortierella sp | 0.9 | 0.1 | 13.6 | 0.3 | 0.1 | 0.1 | 19.0 | 28.8 | 1.8 | 9.7 |
| 26 | | | | | | | | | | | |
| yNI01 | Mortierella sp | 1.0 | 0.2 | 14.1 | 0.5 | 0.1 | 0.1 | 3.6 | 28.5 | 3.0 | 13.5 |
| 27 | | | | | | | | | | | |
| yNI01 | Mortierella sp | 1.0 | 0.2 | 14.5 | 0.5 | 0.1 | 0.1 | 3.7 | 31.9 | 2.3 | 13.8 |
| 28 | | | | | | | | | | | |
| yNI01 | Mortierella sp | 1.0 | 0.2 | 14.5 | 0.5 | 0.1 | 0.1 | 3.8 | 29.7 | 2.8 | 12.8 |
| 29 | | | | | | | | | | | |
| yNI01 | Mortierella sp | 0.9 | 0.2 | 13.4 | 0.4 | 0.1 | 0.1 | 3.1 | 29.2 | 2.4 | 16.7 |
| 30 | | | | | | | | | | | |
| yNI01 | Mortierella | 1.0 | 0.4 | 14.5 | 0.2 | 0.2 | 0.1 | 6.0 | 29.7 | 0.9 | 10.1 |
| 31 | elongata | | | | | | | | | | |

Fatty acid composition of TAG and polar lipids from Mortierella and Mucor isolates from soil samples

| | | | | | | | | | | TAG |
|---|---|---|---|---|---|---|---|---|---|---|
| Strain | | | | C20: | C20:4ω | | | | | or |
| ID | Species | C18:3ω6 | C20:0 | 3ω6 | 6 (ARA) | C22:0 | C22:1 | C24:0 | Rest | PL (%) |
| | | TAG fatty acid composition | | | | | | | | |
| yNI01 | Mucor | 10.3 | 0.6 | 0 | 0 | 0.5 | 0.1 | 0.6 | 1.4 | 17.9 |
| 21 | hiemalis | | | | | | | | | |
| yNI01 | Mucor | 8.9 | 0.3 | 0 | 0 | 0.5 | 0.1 | 0.9 | 1.6 | 14.2 |
| 22 | hiemalis | | | | | | | | | |
| yNI01 | Mucor | 10.1 | 0.2 | 0 | 0 | 0.4 | 0.1 | 0.9 | 1.7 | 14.6 |
| 23 | hiemalis | | | | | | | | | |
| yNI01 | Mucor | 11.1 | 0.2 | 0 | 0 | 0.4 | 0.1 | 0.9 | 1.7 | 12.6 |
| 24 | hiemalis | | | | | | | | | |
| yNI01 | Mortierella | 1.9 | 0.4 | 1.8 | 6.1 | 0.5 | 0.7 | 1.5 | 2.6 | 19.9 |
| 25 | elongata | | | | | | | | | |
| yNI01 | Mortierella | 1.9 | 0.5 | 5.9 | 30.7 | 1.6 | 0.5 | 4.5 | 2.5 | 14.4 |
| 33 | alpina | | | | | | | | | |
| yNI01 | Mortierella | 2.3 | 0.7 | 5.7 | 25.4 | 2.1 | 0.5 | 5.0 | 2.5 | 8.9 |
| 34 | alpina | | | | | | | | | |
| yNI01 | Mortierella | 1.8 | 0.5 | 5.8 | 26.5 | 1.5 | 0.5 | 4.3 | 2.7 | 17.9 |
| 35 | alpina | | | | | | | | | |
| yNI01 | Mortierella sp | 1.0 | 0.4 | 0.8 | 4.0 | 0.6 | 0.7 | 0.4 | 2.8 | 26.2 |
| 26 | | | | | | | | | | |
| yNI01 | Mortierella sp | 1.8 | 0.2 | 1.0 | 7.3 | 0.4 | 1.1 | 0.4 | 3.2 | 17.5 |
| 27 | | | | | | | | | | |
| yNI01 | Mortierella sp | 1.3 | 0.2 | 0.9 | 5.7 | 0.4 | 1.0 | 0.4 | 3.0 | 17.9 |
| 28 | | | | | | | | | | |
| yNI01 | Mortierella sp | 1.9 | 0.2 | 1.0 | 8.2 | 0.4 | 1.1 | 0.3 | 3.3 | 17.6 |
| 29 | | | | | | | | | | |
| yNI01 | Mortierella sp | 1.7 | 0.2 | 0.9 | 5.2 | 0.3 | 1.7 | 0.4 | 3.6 | 20.5 |
| 30 | | | | | | | | | | |
| yNI01 | Mortierella | 2.9 | 0.4 | 2.2 | 11.2 | 0.7 | 2.0 | 1.3 | 3.4 | 16.9 |
| 31 | elongata | | | | | | | | | |
| | | Polar lipid fatty acid composition | | | | | | | | |
| yNI01 | Mucor | 26.0 | 0.1 | 0.1 | 0 | 0.5 | 0.1 | 1.2 | 1.3 | 5.5 |
| 21 | hiemalis | | | | | | | | | |
| yNI01 | Mucor | 29.4 | 0.1 | 0.1 | 0 | 0.5 | 0.1 | 1.8 | 1.5 | 6.4 |
| 22 | hiemalis | | | | | | | | | |
| yNI01 | Mucor | 28.9 | 0.1 | 0.1 | 0 | 0.4 | 0.1 | 1.6 | 1.5 | 7.1 |
| 23 | hiemalis | | | | | | | | | |
| yNI01 | Mucor | 27.0 | 0.1 | 0.1 | 0 | 0.4 | 0.2 | 1.6 | 1.5 | 2.0 |
| 24 | hiemalis | | | | | | | | | |
| yNI01 | Mortierella | 6.5 | 0.1 | 1.8 | 21.4 | 0.2 | 2.0 | 1.4 | 2.0 | 2.6 |
| 25 | elongata | | | | | | | | | |
| yNI01 | Mortierella | 4.0 | 0.5 | 2.8 | 30.1 | 0.3 | 0.9 | 1.5 | 3.0 | 1.7 |
| 33 | alpina | | | | | | | | | |
| yNI01 | Mortierella | 7.3 | 0 | 3.5 | 26.7 | 0.5 | 1.0 | 1.9 | 3.0 | 1.0 |
| 34 | alpina | | | | | | | | | |
| yNI01 | Mortierella | 4.1 | 0.4 | 2.7 | 28.9 | 0.3 | 0.5 | 1.3 | 2.6 | 2.2 |
| 35 | alpina | | | | | | | | | |
| yNI01 | Mortierella sp | 3.9 | 0.1 | 1.2 | 15.4 | 0.2 | 1.7 | 0.4 | 2.9 | 3.0 |
| 26 | | | | | | | | | | |
| yNI01 | Mortierella sp | 6.3 | 0 | 1.2 | 21.6 | 0.1 | 2.5 | 0.4 | 3.2 | 2.3 |
| 27 | | | | | | | | | | |
| yNI01 | Mortierella sp | 4.8 | 0.1 | 1.3 | 19.5 | 0.1 | 2.7 | 0.5 | 3.1 | 2.3 |
| 28 | | | | | | | | | | |
| yNI01 | Mortierella sp | 5.6 | 0 | 1.1 | 21.8 | 0.1 | 2.3 | 0.4 | 3.2 | 2.3 |
| 29 | | | | | | | | | | |
| yNI01 | Mortierella sp | 7.4 | 0.9 | 1.1 | 16.2 | 0.1 | 3.6 | 0.5 | 3.7 | 2.2 |
| 30 | | | | | | | | | | |
| yNI01 | Mortierella | 6.7 | 0.1 | 2.1 | 19.1 | 0.3 | 4.4 | 1.3 | 2.9 | 2.4 |
| 31 | elongata | | | | | | | | | |

Again in contrast to Mucor, the presumed Mortierella sp. strains yNI0126 to yNI0130 produced ARA and DGLA in addition to GLA and accumulated these ω6 PUFA in both TAG and polar lipids. However, in contrast to the M. alpina strains, the Mortierella sp. strains accumulated 2- to 4-fold more ARA in their polar lipid than in their TAG. It was concluded that these Mortierella sp. strains, like the Mucor strains, preferentially accumulated their ω6 PUFA in the polar lipid relative to the TAG. The two Mortierella elongata strains yNI0125 and yNI0131 were similar in many features to the Mortierella sp. strains, including that they produced ARA and DGLA in addition to GLA and accumulated these ω6 PUFA in both TAG and polar lipids. They also showing a preference for accumulated more ARA in their polar lipid than in their TAG. The Mortierella elongata strains could be distinguished from the Mortierella sp. in the levels of some of the other fatty acids, or the ratios between pairs of related fatty acids, i.e. reflecting the conversion rate of one fatty acid to another e.g. GLA to DGLA. Nevertheless, further phylogenetic analyses need to be done to establish the relationship of the Mortierella sp. strains to the Mortierella elongata strains.

All of the strains tested had considerable amounts of monounsaturated and saturated fatty acids in both the TAG and polar lipid fractions. Oleic acid was the most abundant fatty acid in both the TAG and polar lipid fractions of the Mucor hiemalis, Mortierella sp. and Mortierella elongata strains, but not in the Mortierella alpina strains where ARA was the most abundant fatty acid. Palmitic acid was the most abundant SFA in both the TAG and polar lipid fractions in all of the strains examined. With one or two exceptions, the amount of stearic acid was relative low at about 3-10% in TAG and about 2-6% in polar lipid. The other SFA present in all strains were myristic acid (C14:0), pentadecanoic acid (C15:0), arachidic acid (C20:0), behenic acid (C22:0) and lignoceric acid (C24:0). The monounsaturated fatty acids C16:147, C17:1, C18:1Δ11 (vaccenic acid) and C22:1 were present at low but detectable levels in all of the strains.

The inventors next cultured selected strains yNI0121 (Mucor hiemalis), yNI0125 (Mortierella elongata), yNI0127 (Mortierella sp.) and yNI0132 (Mortierella alpina) in order generate larger quantities of fungal biomass to evaluate mycelium disruption methods and to produce sufficient amounts of extracted lipid for food incorporation experimentation. Fungal biomass was also tested in order to determine whether whole cell biomass, either in a wet form or dried as a powder, could be used in Maillard-type reactions to produce meat-like aromas from these fungi containing PL having ω6 fatty acids. This would also allow a comparison of strains that had about equal levels of ω6 fatty acids in the TAG and polar lipid fractions with those that had more ω6 fatty acids in their polar lipids relative to the TAG.

To prepare seed cultures for the larger cultures, the fungal strains yNI0121, yNI0125, yNI0127 and yNI0132 were freshly propagated by agar slice growth, taking 0.5×0.5 cm agar pieces with fungal mycelium from the edge of colonies and placed them in the centre of a fresh MEA plate. The plates were kept at ambient temperature for 3 to 5 days until the new colonies were at least 3 cm in diameter. For each strain, intermediate cultures were then prepared by inoculating six 0.5×0.5 cm agar pieces containing mycelium into 10 ml malt extract medium and incubating these with shaking for 3 days at 26° C. and then kept stationary for 2 days. The complete cultures were then used to inoculate 50 ml of malt extract medium in 250 ml baffled flasks and incubated with shaking at 26° C. for 3 days. These cultures were then used to inoculate 600 ml of medium containing (per litre) 60 g glucose, 10 g yeast extract, 5 g malt extract, 4 g KH₂PO₄, 3 g (NH₄)₂HPO₄ and 0.6 g MgSO₄ with the pH adjusted to 6.0 with 2 M NaOH. These larger cultures were incubated with shaking at 26° C., the cultures sampled after 2 days and the biomass harvested by centrifugation after 3 days, freeze dried and then frozen.

The wet weights and corresponding dry weights for the three Mortierella species in a first culture, and for the three Mortierella species and the Mucor hiemalis strain are shown in Table 48.

TABLE 48
Biomass weights obtained from larger scale
culture of Mortierella strains

| | | | | |
|---|---|---|---|---|
| | 48 h wet | 72 h wet | 48 h dry | 72 h dry |
| Strain | weight (g) | weight (g) | weight (g) | weight (g) |

Experiment 1

| | | | | |
|---|---|---|---|---|
| yNI0125 | nd | 10.3 | 2.81 | 4.91 |
| yNI0127 | 5.27 | 13.7 | 2.96 | 4.64 |
| yNI0132 | 6.08 | 16.7 | 2.64 | 4.3 |

Experiment 2

| | |
|---|---|
| yNI0121 | 38.5 |
| yNI0125 | 13.25 |
| yNI0127 | 10.84 |
| yNI0132 | 15.85 |

### Improved Biomass Production for Fungal Strains

In an attempt to improve biomass weights achieved in the cultures, two culture media were compared. To test a first medium, the three Mortierella isolates yNI0125, yNI0127 and yNI0132 and Mucor hiemalis strain yNI00121 were grown in a seed culture containing (per litre) 20 g glucose, 6 g yeast extract, 5 g malt extract, 3 g KH₂PO₄, 3 g (NH₄)₂HPO₄ and 3 g MgSO₄·7H₂O. The seed cultures were used to inoculate 600 ml cultures in Medium 1 (per litre): 20 g glucose, 5 g yeast extract, 10 g peptone, incubated at 26° C. with shaking at 200 rpm for aeration. Parallel cultures of 800 ml were also grown at the same time in a second medium, Medium 2, containing 30 g glycerol, 0.85 g yeast extract, 8.7 g KH₂PO₄, 1.9 g (NH₄)₂HPO₄, pH 6.2, cultured at 26° C. with shaking at 200 rpm for aeration. Growth was significantly faster in Medium 1, reaching about 14 g/l dry weight at 70 h.

Dried whole cell biomass from these strains were used in Maillard reactions (Example 7).

Extraction of Total Lipid from Fungal Biomass

Total lipid was extracted from harvested wet fungal biomass (Table 48, Experiment 2) using hexane as solvent, as follows. Most of the water was removed by washing the cell biomass with ethanol, using 2 ml of ethanol per gram of cell biomass (wet weight) followed by centrifugation each time to recover the cell biomass. The pelleted cells were resuspended in hexane, using 5 ml hexane per gram of cell biomass. The suspensions were homogenised and the cells disrupted with the UltraTurrax (IKA, Malaysia) for 3 min followed by sonication for 5 min, which pair of treatments was repeated twice for a total of three times. The mixtures were shaken for 3 h at room temperature, although later experiments showed that shaking the mixtures overnight extracted more lipid. Observation by microscopy of samples from the mixtures showed that many but not all of the cells had been disrupted by the treatments. The mixtures were centrifuged and the hexane phase collected. The hexane was evaporated from each extraction using a flow of nitrogen, and the dried lipid extracts weighed. This resulted in 0.99 g from yNI0121 (Mucor hiemalis), 1.33 g from yNI0125 (Mortierella elongata), 0.69 g from yNI0127 (Mortierella sp.) and 0.78 g from yNI0132 (Mortierella alpina). Samples of these lipids were chromatographed on TLC plates and the polar lipids extracted from the silica for analysis of the fatty acid composition by GC of FAME as described in Example 1.

Extraction of Partially Purified Polar Lipid from Fungal Biomass

An alternative extraction method was tested as a means of preferentially extracting polar lipids by extraction into ethanol, based on the greater solubility of polar lipid in ethanol relative to neutral lipids. Harvested dry biomass following fermentation of Mortierella alpina (45.63 g) and 60 mL of ethanol were blended and at least partially disrupted using an UltraTurrax homogeniser. The sample was then mixed with stirring for 30 min and centrifuged. The ethanol supernatant was removed. This extraction of the M. alpina biomass with ethanol was repeated twice and the supernatants combined. The precipitate can be retained for extraction of neutral lipids if desired. The ethanol was evaporated from the combined supernatants in a rotary evaporator, programmed as follows: vacuum pump at 15 mbar, chiller at −16° C., water bath at 37° C. and 400 rpm. From an initial input of 45.63 g of M. alpina dry biomass, 5.7 g of phospholipid enriched precipitate was recovered. The precipitate at this stage also contained some TAG. The phospholipid enriched precipitate was dissolved in 30 ml hexane and cooled in an ice bath at 0° C. Next, 120 ml of cold acetone (−20° C.) was added into the stirred mixture to precipitate phospholipids. The precipitate was washed 5 times with 30 ml portions of cold acetone (−20° C.). The residual solvent in the extracted and purified phospholipid preparation was removed in a rotary evaporator at room temperature for 10 h. The polar lipid yield was measured gravimetrically and a small aliquot used for FAME analysis. Another aliquot was chromatographed on TLC to check for purity. From an initial input of 45.63 g of M. alpina dry biomass, 1.1 g of relatively pure phospholipid was recovered.

### Example 22. Production of Fungal Biomass at Larger Scale

The inventors next produced whole cell biomass and lipid extracts from the biomass including PL containing ω6 fatty acids such as ARA from the fungal isolates described in Example 6. The fungal isolates were cultured at 35 L scale, the fungal mass harvested from the cultures and lipids extracted. In some experiments, the lipids were fractionated to isolate the polar lipids, including the PL, and both whole cells and extracted lipids used in Maillard reactions and food preparations.

### Larger Scale Production of Fungal Biomass and Extraction of Lipids Having ω6 Fatty Acid (B017)

In a larger scale experiment producing 35 L of culture, Mortierella alpina strain yNI0132 was grown in a Braun fermenter in a rich medium containing glucose as the main carbon source, seeking to produce more cell biomass and a suitable polar lipid: TAG ratio having ω6 fatty acid incorporated into polar lipids. The growth medium was based on a rich yeast extract-malt extract medium which favoured biomass production rather than TAG production, even though M. alpina is an oleaginous species that naturally is capable of producing abundant TAG. The medium used for the seed culture for inoculation and for the first phase of culture contained (per litre) 60 g glucose, 10 g yeast extract, 5 g malt extract, 3 g (NH₄)₂SO₄, 1 g KH₂PO₄, 0.6 g MgSO₄·7H₂O, 0.06 g CaCl₂) and 0.001 g of ZnSO₄, pH 6.2. The second stage of culturing used a feed solution of 5 L containing (per litre) 5 g malt extract, 7.5 g (NH₄)₂SO₄, 1 g KH₂PO₄, 6.0 g MgSO₄·7H₂O, 0.3 g CaCl₂) and 0.005 g of ZnSO₄ but no yeast extract. These media used ammonium sulphate as the nitrogen source rather than urea. The first phase culture medium was prepared and sterilised in the fermenter by autoclaving in situ at 121° C. for 15 min, then cooled by direct cooling to the fermenter jacket. The glucose stock solution (438 g glucose monohydrate plus 563 ml water) was autoclaved separately as a 40% solution and, while still warm at 45° C., was added to the fermenter.

An inoculum culture was prepared in 4×200 ml YM broth in 500 ml flasks using starter cultures from agar plates. The inoculum culture was incubated for 71.5 h at 30° C. with shaking at 180 rpm, at which time the inoculum cultures showed luxuriant growth. The inoculum culture was introduced into the fermenter without homogenisation of the culture. In the first phase of culturing with the aim of maximising biomass production, a high aeration rate was maintained at about 0.6 to 1.0 vvm (18-30 l/min) and mixing was low at 50-150 rpm to maintain dissolved oxygen at greater than 1 ppm without excessive shear forces being applied to the culture. After 76 h cultivation, the nutrient feed solution was added to the fermenter. The pH was controlled at 6.0 throughout by addition of NaOH and the temperature was maintained at 30° C. The culture was sampled (50 ml) every 24 h post inoculation. The parameters that were measured daily were cell density (dry cell weight), glucose level by HPLC, total nitrogen level by the Kjeldahl method, phosphate and sulphate levels by colorimetric strips, and the appearance of the fungus by light microscopy. Dry weight (dry cell weight) was measured by weighing the material collected on a glass microfibre obtained by filtering 20 ml of culture using a Buchner funnel and a vacuum pump before being dried in an oven and then weighed. The culture was harvested at 94 h when the cell density had reached 19.4 g/l (wet weight/w; Table 49). The biomass was harvested by filtration through a nylon gauze (200 micron). The biomass was resuspended and washed twice, each time with two volumes of cold water relative to the volume of biomass. The mycelial biomass was grey-white in colour. Excess water was removed by squeezing the wet mycelial cake through the filter cloth by hand. This yielded 2.27 kg of washed biomass having a dry weight of approximately 590 g. The biomass cake was spread to a 1-2 cm layer in ziploc bags and frozen.

TABLE 49
Raw data from fermentation experiment B017 for
M. alpina strain yNI0132

| | | | Phos- | | | Glucose | Nitro- |
|---|---|---|---|---|---|---|---|
| Time | Glucose | Sulphate | phate | DW | Nitro- | (% | gen |
| (h) | (g/l) | (mg/l) | (mg/l) | g/l | gen | start) | (% start) |
| 0 | 59.6 | 400 | 250 | 0.47 | 0.5 | 100 | 100 |
| 24.2 | 56.7 | 400 | 250 | 2.0 | 0.49 | 95 | 98 |
| 46.0 | 40.2 | 400 | 250 | 15.8 | 0.07 | 67 | 14 |
| 70.3 | 38.4 | 400 | 250 | 17.0 | 0 | 64 | 0 |
| 94.0 | 23.4 | 400 | 250 | 19.4 | 0.03 | 39 | 6 |

As indicated by the DW and pH, most of the fungal growth occurred between 20 and 50 h. The culture reached a stationary phase at about 50 h, presumably due to depletion of nitrogen, with no further pH adjustment occurring after that time point. Nitrogen by the Kjeldahl method was depleted at 70.3 h. Further addition of nitrogen at 76 h by the feed solution provided a further increase in glucose consumption and a further increase in DW of the biomass. The final glucose concentration was 23.37 g/l, so only 60% of the initial amount was consumed. Further adjustment of the nitrogen to carbon ratio was therefore considered to optimise biomass production.

### Example 23. Maillard Reactions Using Fungal Biomass and Extracted Lipid

The inventors tested the M. alpina cells and the extracted lipid obtained from the cells, enriched for polar lipid and containing the ω6 fatty acids ARA, DGLA and GLA, in Maillard reactions. The experiment also tested a combination of cells and the extracted lipid, all produced as described in Example 7. These reactions had L-cysteine, D-ribose, thiamine hydrochloride, iron fumarate and glutamic acid present in a phosphate buffer at pH 6.0, and either had added yeast extract or lacked the yeast extract. These reactions were intended to approximate the use of flavouring mixes having multiple components which are often added to food preparations for flavouring and other sensory attributes. The presence or absence of yeast extract was intended to test whether it would either mask, or enhance, the aroma produced by the M. alpina cells or extracted lipid having PL, or have little effect.

The base medium used for the Maillard reactions, designated “Matrix A” lacking yeast extract and “Matrix B” including yeast extract, had the following composition in aqueous buffer at final concentrations: 10 mM L-cysteine, 10 mM D-(−)-ribose, 2 mM thiamine hydrochloride, 35 μg/ml of iron fumarate (Apohealth, NSW, Australia) and 2 mM L-glutamic acid monosodium salt hydrate. These components were dissolved in 32.6 mM potassium phosphate buffer pH 6.0 for Matrix A or 12.6 mM phosphate buffer, pH 6.0 for Matrix B, prepared from potassium dihydrogen phosphate and dipotassium hydrogen phosphate. Yeast extract was added to Matrix B at a final concentration of 30 mg/ml. Reactions were carried out in 2 ml volumes in 20 ml glass vials with tightly sealing screw top lids. The reaction mixtures were made up with M. alpina dry biomass (150 mg) or extracted polar lipid (20 mg, 50 mg or 70 mg), or a combination of cells and lipid as indicated in Table 50. As controls for the presence of M. alpina biomass or polar lipid, other vials were made up with 150 mg of S. cerevisiae cells or 70 mg extracted lipid from the S. cerevisiae cells, both of which did not contain ω6 fatty acids (Reactions #8 to #12). Additional reaction mixtures (Reactions #5, #9) were prepared and vortexed, but then frozen overnight before being thawed and heat treated with the other reaction mixtures.

The mixtures were vortexed vigorously for 2 min and then heated for 75 min in an oven set at 140° C. The vials were tightly sealed during the heat treatment. It was estimated that the samples took about 15 min to warm to the oven temperature, so the heat treatment included about 60 min at 140° C. The vials were cooled until warm to the touch about 15 min later, and then opened briefly for sniffing by a panel of 5 volunteers (P1 to P5). These included males and females and ranged from 24-65 years in age. The volunteers did not know the composition of any of the vials prior to sniffing the contents and the vials were sniffed in a random order as selected by the volunteers. Their descriptions of the aromas were recorded without any comments being shared; the data are shown in Table 51. The descriptions of the aromas for reactions #4 to #7 were combined in Table 51 while still indicating any preference within reactions #4 to #7. The responses to reactions #8 to #11 were similarly combined for volunteers P3 and P4.

Reactions #4 to #7 containing M. alpina biomass and/or extracted lipid were described by all five volunteers as having a meaty aroma, but with different aroma notes recorded by the volunteers, whilst the descriptions of the aromas from reactions having the S. cerevisiae biomass were more variable between the volunteers. The control reaction mixtures lacking the lipid extract, and the mixtures having the lipid extract without any cell biomass, were generally perceived to have a lower intensity of aromas compared to the corresponding samples that contained biomass or a combination of biomass and extracted lipid from M. alpina. Reaction mixtures containing biomass spiked with the extracted lipid from M. alpina were described as having similar or enhanced aromas compared to reactions containing only M. alpina biomass. Mixtures that had been frozen and thawed and then heated resulted in similar aroma responses to freshly prepared mixtures heated in the same manner. The inventors concluded that the M. alpina biomass, containing polar lipids incorporating ω6 fatty acids, provided meaty aromas, particularly for beef aromas such as roast beef. Extracted lipid enriched for PL containing ω6 fatty acids also provided meaty aromas, enhancing the aromas when applied with the biomass. When used without the cell biomass, the responses for the extracted lipids were weaker but this could be countered by applying larger amounts of the lipid.

TABLE 50
Composition of Maillard reactions using
fungal biomass or extracted lipid

| Reac- | | | | |
|---|---|---|---|---|
| tion | Ma- | | | Freeze- |
| ID | trix | Cell biomass | Extracted lipid | thaw? |
| 1 | A | none | none | No |
| 2 | B | none | none | No |
| 3 | B | none | 70 mg from M. alpina | No |
| 4 | B | 150 mg M. alpina | none | No |
| 5 | B | 150 mg M. alpina | none | Yes |
| 6 | B | 150 mg M. alpina | 20 mg from M. alpina | No |
| 7 | B | 100 mg M. alpina | 50 mg from M. alpina | No |
| 8 | B | 150 mg S. cerevisiae | none | No |
| 9 | B | 150 mg S. cerevisiae | none | Yes |
| 10 | B | 120 mg S. cerevisiae | 30 mg from S. cerevisiae | No |
| 11 | B | 100 mg S. cerevisiae | 50 mg from S. cerevisiae | No |
| 12 | B | none | 70 mg from S. cerevisiae | No |

TABLE 51

| Reaction | | | | | |
|---|---|---|---|---|---|
| ID | P1 | P2 | P3 | P4 | P5 |
| 1 | Some light | Pork belly | Similar | Meaty smell, | Blank, pleasant |
| | meaty aroma | | between #1- | a bit stronger | |
| | | | 2 cannot | than #2 | |
| 2 | Some light | Pork belly | relate to a | Meaty smell | Blank, pleasant |
| | meaty aroma | | specific aroma | | |
| | (lighter than #8) | | | | |
| 3 | Pleasant aroma, but | Light roast | Meaty aroma, | Light beef | Vegetable and chicken |
| | not a distinguished | meat | but do not | aroma | |
| | aroma note | | like much | | |
| 4 | Roast meat, | Roast meat, | Meat aroma, | Beefy aroma, | Meaty aroma, very |
| 5 | similar | similar for | very | similar | nice and pleasant. #5 |
| 6 | between | #4 to 7 | pleasant, | between #4 | is better than #4, #6 is |
| 7 | reactions #4 | | quite similar, | to 7 | the best, #7 was pleasant, |
| | to 7 | | but like #4 | | but not as good as #6, |
| | | | the most | | somewhat like charred meat |
| 8 | Light meat | Meaty, chicken, | Meaty smell, like | Beef cooked | Stew meat |
| | aroma | similar to #9 | #10 the best | with mushroom | |
| 9 | Light meaty | Chicken, meaty, | | however, there | Light meat, chicken |
| | aroma | pleasant | | are some slight | like flavour |
| 10 | Meaty and | Medium beefy | | differences: | Good roast beef/pork |
| | buttery | | | #11 smoky, | |
| 11 | Meaty and | Medium beefy, | | #12 strongest | Similar aroma note with |
| | buttery | similar to #10 | | | #10, but slightly lighter |
| 12 | Light meaty | Beefy, pork | | | Vegetables |
| | aroma | lard smell | | | |

Several experiments were carried out to test variations of the Maillard reactions in terms of the composition of the base medium. In one experiment, four different base media were prepared either with or without the L-glutamic acid at 5 mM, or with or without an added Fenugreek (Trigonella foenum-graecum) leaf powder at 10 mg per 2 ml reaction. Fenugreek leaf powder was tested as this herb has long been used in food cooking to enhance the flavour of dishes such as in curries or in combination with other herbs or spices such as cumin and coriander. All of the base media included a yeast extract at 30 mg/ml. The reactions were set up including Y. lipolytica cells incorporating ARA in its polar lipid (cells from experiments B012 or B013) or M. alpina cells. The cells were applied as wet cells at 200 mg per 2 ml reaction in 20 ml glass vials, tightly sealed. Control reactions had the same base media compositions but lacked the Y. lipolytica or M. alpina cells. The reaction mixes were sonicated as a batch by placing all vials in a floating foam and placed in a sonicator (Soniclean, Thermoline) set up at a medium power for 30 min and then heat treated in an oven at 140° C. for 60 min. The vials containing the reaction mixtures were cooled slowly over about 15 min until warm to the touch. The contents were sniffed in random order by nine volunteers who did not know the composition of each mixture. The reactions had been coded with random 3-digit numbers to avoid bias, and the volunteers sniffed coffee beans between samples to reset the olefactory senses.

Although the descriptions of the aromas were variable between the nine volunteers, the aromas from mixtures having glutamic acid were generally described as more associated with meaty aromas compared to the reactions lacking glutamic acid. For example, a reaction mixture having glutamic acid was described as providing meaty aroma by 5 of the 9 participants whereas the corresponding sample lacking glutamic acid was described as having a meaty aroma by only 2 participants. Addition of fenugreek leaf powder in the reactions was generally described as generating a pleasant, sweet herb or vegetable aroma, but addition of the herb powder also moderated the meaty aroma in the presence of the Y. lipolytica or M. alpina cells. Some of the participants described the meaty aromas as “roast chicken”, “chicken broth” or “crispy chicken”, so identifying the aromas as like chicken in some form.

In another experiment, samples were prepared in either Matrix A or Matrix B as the base medium and containing either 100 mg wet M. alpina cells or 15 mg extracted lipid enriched for polar lipid. These reaction mixtures were prepared in 1 ml volumes in 20 ml glass vials and were each vortexed vigorously for 2 min. The mixtures were heat treated as before. Parallel mixtures were heated at a lower temperature, namely 115° C. for 25 min. The responses from three volunteers were consistent with the other experiments, in that the aromas generated by the whole cell biomass generated stronger meaty aromas than the extracted lipid on its own. The samples treated at 115° C. for the shorter time were evaluated as providing a weaker or lighter aroma, indicating that the treatment at 140° C. was more efficient at generating the meaty aroma than treatment at 115° C.

In another experiment, the M. alpina biomass as a dried powder was compared to several commercial plant-based and meat flavouring products on the market in Australia, including Deliciou plant-based beef, Deliciou plant-based chicken, Deliciou plant-based pork, Massel plant-based stock cube-beef, Massel plant-based stock cube-chicken, Oxo stock cube-beef, Oxo stock cube-chicken and Bonox beef stock. Reaction mixtures were prepared in 2 ml volumes using 150 mg of dry product or 200 mg of product as a wet paste and heated at 140° C. for about 60 min. When sniffed by four volunteers, the samples containing the M. alpina cell biomass were described as comparable or superior in their meaty aroma to the commercially-available flavouring products.

In another experiment, reaction mixes were prepared and then dried down by placing the vials in an oven at 115° C. for 2 h followed by 82° C. for a further 2 h. In a parallel experiment, corresponding samples were dried overnight at 70° C. After the heating, all of the samples were reconstituted in 2 ml of water, mixing them well to dissolve the dried powder, and subjected to sniffing by volunteers. The samples treated at the higher temperature generally provided a burnt smell, whereas the samples subjected to the lower temperature drying still provided some meaty aromas. This indicated that lower temperature drying was better than the higher temperature for retaining the meaty aroma. Further investigation is carried out to optimise the drying conditions.

The inventors concluded that a variety of compositions can be used with the yeast or fungal biomass containing ω6 fatty acids to enhance meaty aromas when heated, including in the presence of other flavouring components as commonly used in food preparations.

### Example 24. Further Maillard Reactions Using Fungal Biomass and Extracted Lipid

The inventors further tested the M. alpina cells and the extracted lipid obtained from the cells in further Maillard reactions under modified conditions. From the previous experiments, the samples containing M. alpina biomass were considered to have the strongest meat-like aroma, often described as having a roast meat/BBQ meat aroma. Several volunteers in the aroma tests, however, described that to them the aroma was like an overcooked or even burnt meat with a charred note. A “fatty aroma” was also noted by some. In another experiment, when the mixtures were tasted after heating, some volunteers described a sourness or bitterness in the samples including the matrix bases A and B, in particular bitterness for samples containing M. alpina. By tasting the individual solutions and ingredients to make up the matrix bases A and B, it was concluded that the thiamine hydrochloride contributed to the bitterness and to a lesser extent the yeast extract solution. The iron and cysteine solutions, both dissolved in 1 N HCl, contributed to the sourness. Several new experiments using different approaches were therefore performed to improve both aroma and taste, aiming to reduce the burnt smell as well as the sourness and bitterness but retaining or even enhancing the meat-like aroma.

### Experiment 1

In this experiment, the M. alpina biomass was partially substituted with S. cerevisiae cells that did not contain ω6 fatty acids to see whether the burnt smell of the mixture was reduced after heat treatment compared to M. alpina alone. To do this, samples were prepared containing 150 mg of either dry M. alpina cells or S. cerevisiae cells in 2 ml of matrix B, or 75 mg of each of the fungal biomasses. The samples were heated in an oven set at 140° C. for 75 min. As before, the M. alpina sample generated a roast meat aroma, while the S. cerevisiae sample generated more of a gravy meat or chicken broth aroma. The mixed sample produced an intermediate aroma of roast and gravy meat. The volunteers described that the burnt smell was lessened for the mixture, but a decreased roast meat aroma was also noted. The sourness and bitterness were still found in all samples. The results suggested that some of the M. alpina biomass could be replaced with other cells to generate an intermediate meaty aroma more like a roast beef or gravy.

### Experiment 2

In this experiment, an alternative base medium was used to compare it to the Matrix B base. This alternative medium contained a mixture of amino acids, including cystine (33%), glutamine, alanine, leucine, glutamic acid, lysine, valine, proline and methionine as well as 2.7% dextrose by weight. This mixture was added at 7.5% (w/v) to the aqueous medium, as was an additional 0.5% (w/v) cystine and 0.5% (w/v) dextrose. The samples for the Maillard reactions used either 150 mg of dry M. alpina biomass or 300 mg of wet slurry of S. cerevisiae cells. Control samples had only the amino acids and sugars and no cells added. These mixtures were heated in an oven at 140° C. for 75 min. The control sample having Matrix B was described as having a light meaty aroma and some umami after taste, but was also immediately perceived as having sourness and bitterness. In contrast, the samples containing M. alpina generated a meaty aroma. The control sample having the alternative base medium without fungal biomass had a pleasant aroma which was not related to a specific type of meat. When the M. alpina biomass was added, it generated different meaty notes and an umami/sweet taste perceived as an after taste. A slight sourness and bitterness was still perceived in these samples. It was considered that the slight sourness and bitterness could be masked by increasing the amount of dextrose.

### Experiment 3

In another experiment, the alternative base medium was used at two concentrations: 7.5% (w/v) or 0.75% (w/v). Another sample had an additional 100 mg dextrose added per 2 ml mixture. Some samples contained 200 mg of extracted polar lipid, mostly PL, from M. alpina. A shortened heat treatment of 45 min at 140° C. was applied for samples containing M. alpina while the standard heat treatment of 75 min at 140° C. was used for other samples. The volunteers described that the mixtures having the higher concentration of base medium had a more distinguished meat-like and pleasant aroma compared to the samples prepared at the low concentration. Further, the higher concentration samples had a browny/golden brown colour after the heat treatment, whereas the lower concentration samples did not have that colour. Based on these results, the future experiments used the higher concentration of base medium. The samples with the PL isolated from M. alpina generated a weaker, but “purer” meaty aroma compared to the use of M. alpina biomass at 150 mg/2 ml. Significantly, heating the samples containing M. alpina for 45 min rather than 75 min reduced the burnt smell and substantially reduced the bitterness. Increasing the amount of dextrose also decreased the perception of bitterness, although some was still noted.

### Experiment 4

This experiment compared the use of a wet form of the fungal biomass compared to the dry form. In this experiment, the sample containing M. alpina dry biomass based on the findings of the previous experiments had a roast aroma with no burnt smell and a light bitterness after the heat treatment. The sample with wet biomass generated a pleasant roast meat aroma with no hint of a burnt smell and a very subtle bitterness similar to the control samples lacking the biomass. This subtle bitterness was similar to the taste of the control mixture having only the base medium, most likely due to its amino acid constituents or the thiamine hydrochloride. It was considered that the increased moisture in the biomass had slowed down the burning process when the biomass was exposed to the high temperature treatment, but still provided sufficient conditions for a Maillard reaction to occur.

### Example 25. Food Products Using Fungal Biomass and Extracted Lipid

The inventors next tested the yeast and M. alpina cells and the extracted lipid obtained from the cells in exemplary food products to test their aroma and taste. The chemicals and ingredients used for the taste mixtures included L-cysteine hydrochloride monohydrate (Fermopure, Wacker, Germany), D-ribose (Epin Biotech Co, China), thiamine hydrochloride (Chem Supply, SA, Australia), monosodium glutamate (Ajinomoto), Yeast extract (Sigma) and an amino acid/sugars blend (provided by V2Foods). The oils and plant-based fats used were canola oil, “Heart Smart” safflower cooking oil and copha vegetable shortening from a supermarket and a plant-based ghee (Emkai Lite Interesterified vegetable fat, Sai food products, Gujarat, India). The food items tested by applying the taste mixtures were a macro firm tofu obtained from a local supermarket, dried bean curd (tofu skin, Shenzhen Ming Lee Food Manufacturing Co. Ltd., Guandong Province, China), a plant-based mince (V2 Foods, Australia) and textured vegetable protein high fibre slices (TVP, Lamyong, NSW, Australia). The fungal biomasses used were a wet slurry of S. cerevisiae having about 10% ARA (B013, see Example 4), or M. alpina biomass in either a wet or dry form (Example 7).

### Experiment 1

This experiment used the B013 yeast biomass, containing ARA in both the polar lipid and TAG (Example 4). A mixture (mixture A) was prepared containing 2 ml of a Matrix B2 base medium. Matrix B2 containing one tenth the concentration of thiamine hydrochloride compared to Matrix B but otherwise had an identical composition. Mixtures were prepared having 0.5 ml of B013 cell slurry and 0.5 ml of a chicken flavoured yeast extract (2.5 g/3 ml water, Flavex). Control mixtures lacked either the B013 cell slurry or the Matrix B2 base medium. Tofu pieces were marinated in the mixtures for 45 min and cooked on a baking tray in an oven set at 1800C for about 6 min. When smelt and tasted, all of the tofu pieces had a salty/sweet/umami taste but only the test pieces treated with mixture A exhibited a light roast chicken aroma and taste. It was considered that the umami taste was most likely brought by the flavoured yeast extract whereas the B013 yeast biomass contributed to the chicken aroma.

### Experiment 2

It was considered that cooking the tofu pieces for only 6 min was not long enough to induce a complete Maillard reaction with the mixtures used, so, in a following experiment, the basting mixtures were heated at 140° C. for 75 min prior to application to the tofu pieces. A taste mixture was prepared containing the B013 yeast biomass having ARA in its lipid. 2 ml Matrix B2 was mixed with 300 mg of wet yeast biomass. This taste mixture was then heated in an oven set at 140° C. for 75 min. Tofu pieces and tofu skin pieces were marinated in 1 ml of the taste mixture for 1 h and then oven baked at 180° C. for 6 min. A meaty aroma was perceived during the marination step and before putting the sample into the oven. However, after heating in the oven, the meaty aroma was no longer perceived. It was concluded that the volatile compounds that imparted the meaty aroma had evaporated during the heating in the oven.

### Experiment 3

In this experiment, the yeast biomass was substituted with 200 mg of M. alpina wet biomass, having about 30% ARA in its lipid. The composition of the mixtures and baking conditions were otherwise the same as in Experiment 2 except that the mixtures were heated for 45 min rather than 75 min prior to application to the tofu pieces. After heating them in the oven, the tofu pieces were sniffed and tasted. The volunteers described that the control tofu marinated in Matrix B2 without the M. alpina biomass had a pleasant, light meaty aroma, whereas the tofu treated with the mixture having the M. alpina biomass had a strong meaty aroma and taste.

### Example 26. Effect of Matrix Component Concentration on Meaty Aroma and Taste

Matrix C (defined in Table 53, below) was mixed at different levels of dilution with the same concentration of wet M. alpina biomass (10% w/v) so as to assess the effect of different amounts of matrix components to the generation of aroma and flavour. The six samples prepared are provided in Table 54 below. The associated compositions of matrix C in each of the samples is provided in Table 55 below.

TABLE 53
Composition of matrix C.

| Stock solutions | Volume (μL) |
|---|---|
| Cysteine•HCL (400 mM) | 250 |
| Ribose (400 mM) | 250 |
| Thiamine•HCL (44 mM) | 90.9 |
| Yeast extract (general) (30 g/100 mL) | 1000 |
| Monosodium glutamate (400 mM) | 125 |
| Water | 284.1 |
| Total | 2000 |

TABLE 54
Composition of samples

| | Matrix | Wet biomass (g) |
|---|---|---|
| Sample 1 | 2 mL matrix C- undiluted | 200 mg |
| Sample 2 | 2 mL matrix C- 2x diluted | 200 mg |
| Sample 3 | 2 mL matrix C -4x diluted | 200 mg |
| Sample 4 | 2 mL matrix C - 8x diluted | 200 mg |
| Sample 5 | No matrix | 200 mg |
| Sample 6 | 2 mL matrix C- undiluted | No biomass |

TABLE 55
Composition of matrix C at different dilution levels for samples 1-6 (amounts shown in μL)

| | Sample 1 | Sample 2 | Sample 3 | Sample 4 | Sample 5 | |
|---|---|---|---|---|---|---|
| | Undiluted + | 2x diluted + | 4x diluted + | 8x diluted + | No matrix + | |
| Stock solutions | 1% | 1% | 1% | 1% | 1% | Sample 6 |
| (μL) | biomass | biomass | biomass | biomass | biomass | Undiluted |
| Cysteine. HCL | 250 | 125 | 62.5 | 31.25 | 0 | 250 |
| (400 mM) | | | | | | |
| Ribose (400 | 250 | 125 | 62.5 | 31.25 | 0 | 250 |
| mM) | | | | | | |
| Thiamine. HCL | 90.9 | 45.45 | 22.72 | 11.36 | 0 | 90.9 |
| (44 mM) | | | | | | |
| Yeast extract | 1000 | 500 | 250 | 125 | 0 | 1000 |
| (general) (30 | | | | | | |
| g/100 mL) | | | | | | |
| Monosodium | 125 | 62.5 | 31.25 | 15.62 | 0 | 125 |
| glutamate (400 | | | | | | |
| mM) | | | | | | |
| Water | 284.1 | 1142.05 | 1571.03 | 1785.5 | 2000 | 284.1 |
| Total | 2000 μL | 2000 UL | 2000 μL | 2000 HL | 2000 μL | 2000 μL |

The samples were vigorously mixed for 2 minutes at room temperature and subjected to heating at 140° C. for 45 minutes. After the heat treatment completed, the samples were cooled down and tempered at 45° C. throughout sensory evaluation.

For sensory evaluation of the samples, a total of six participants (both male and female, aging from 25-65) were asked to sniff and taste the samples in order of 5 to 1 and then 6. Between samples, the participants were asked to sniff coffee and drink water to neutralize/clear the nose and tongue. The participants were asked to evaluate both aroma and taste for the meatiness and pleasantness based on a five-point hedonic scale, with the higher score indicating increased meatiness and pleasantness.

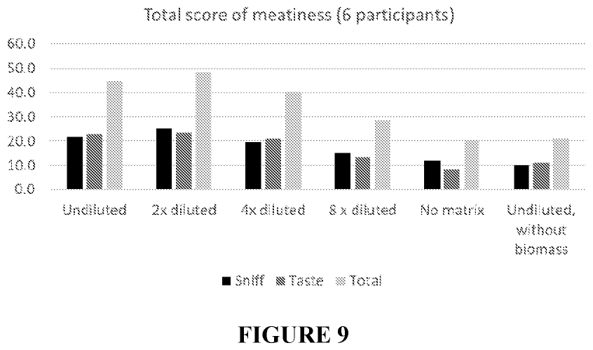

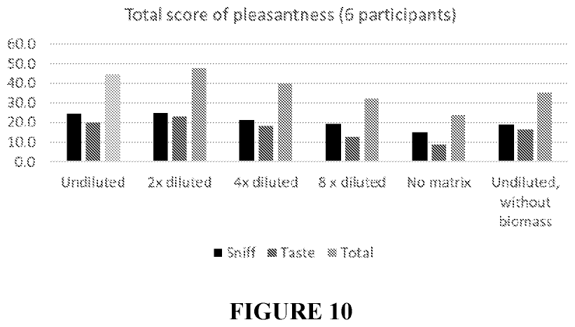

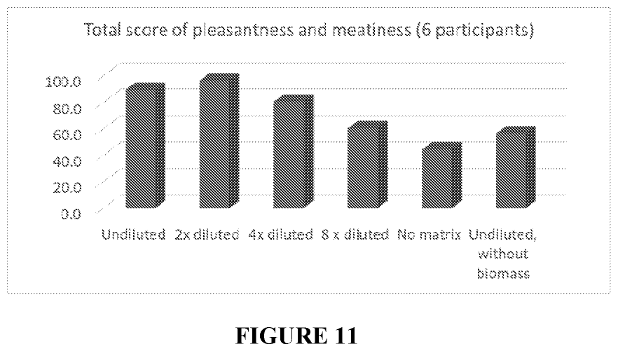

The total score of 6 participants for meatiness, pleasantness and combined pleasantness and meatiness are presented in FIGS. 9, 10 and 11 respectively. It was found that a slight increase in meatiness and pleasantness of the sample was observed when the matrix was diluted 2×, but further dilutions of matrix more than 2× decreased both meatiness and pleasantness of the samples.

### Example 27. Additional Maillard Reaction Comparing Polar Lipid to Neutral Lipid

The inventors further tested the polar and neutral lipid fractions from M. alpina cells in Maillard reactions.

### Polar Lipid Extraction

To extract the polar lipid fraction, 53.2 g of wet M. alpina biomass was weighed into a ziploc bag and left in warm water to defrost. Once defrosted this was homogenised with the handheld T10 ultra turrax homogeniser on speed 5 for 5 min. The biomass was then transferred to a 1 L schott bottle, 650 mL ethanol was added and the bottle was placed on a magnetic stir plate at RT for 15 min. The biomass was filtered by a buchner funnel using Filtech grade 1839 (150 mm) filter paper. The ethanol filtrate was retained and filtered for a second time with filter paper no. 6 watmans paper (finer than the other one). The de-watered biomass was left to dry in the fume hood overnight, once dried this weighed 9.94 g. The dried, de-watered biomass was then extracted in 600 mL Chloroform/Methanol (2:1) in a 2 L schott bottle for 3.5 hrs, before being filtered as done previously. The biomass was left to dry for 3 hrs in a large beaker in the fume hood, and was then placed in a 50 mL falcon tube and weighed (6.423 g). The filtrate was evaporated on the rotor evaporator to collect the extracted lipids. To weigh, the lipids were scraped from the collection flask and transferred to a glass sample vial. The collection flask was rinsed with approximately 5 mL chloroform and sonicated to dissolve all lipid. The chloroform/lipid volume was poured into the sample vial and this rinse was repeated. The sample vial was then dried down to completion under N2. The resulting lipid weighed 2.53 g.

2.0 g of the Chloroform: Methanol (2:1)-extracted lipids was dissolved in 10 mL chloroform and vortexed (15 secs) and sonicated (30 secs) three times to ensure lipids were dissolved. A 10 g HyperSep Aminopropyl SPE column (Thermo Scientific) was conditioned with 80 mL hexane, maintaining a flow of roughly one drip per second. The resuspended lipid extract was loaded onto the column (a 20% lipid/sorbent (w/w) loading of the column), and the through was collected. The neutral lipid was eluted with 80 mL Chloroform which was collected in four 20 ml elutions. The FFAs and polar lipid were eluted in the same manner but with 80 mL Diethyl Ether: Acetic Acid (98:2) and Methanol: Chloroform (6:1) respectively.

Each elution was checked for purity and lipid content on a HPTLC plate. 50 μL of each elution was loaded, alongside 1 mg of the starting material as a control (ie the chloroform: methanol extracted lipids). The polar lipid elutions were then pooled and dried under vacuum on the rotor evaporator. The dried lipids were transferred to a pre-weighed sample vial and the round bottomed rotor evaporator flask was rinse 5 mL of the elution solvent (vortex and sonicate repeatedly to dissolve any dried down lipids). This rinse was added to the sample vial and dried to completion under nitrogen gas. This was the polar lipid fraction.

In order to check the composition of the polar lipid sample, it was run in a second HPTLC check using a two solvent system. 2 mg of the dried Polar Lipid fraction was resuspend in Ethanol, while 2 mg Chloroform: Methanol (2:1) extracted lipid was resuspend in chloroform. 1 mg of each was loaded onto two HPTLC plates, along with 50 μL of the FFA fraction elution 1 and 5 μL of the neutral lipid fraction elution 1 from the SPE experiment above. Both plates were run in the single solvent system (70:30:1 Hexane: Diethyl Ether: Acetic Acid) for 10 cm. One of the plates was then run in the second solvent system (68:22:6:4 Chloroform:methanol:acetic acid: water) for 20 cm. Both plates were sprayed with Primulin and visualise under UV.

### Neutral Lipid Fraction

To extract neutral lipid, 90 mg of hexane extracted lipids was resuspended in 1.125 mL of Chloroform. The sample was briefly vortexed before proceeding. A 2 g HyperSep Aminopropyl SPE column (thermo fisher) was conditioned with 20 mL Hexane and 1 mL (80 mg) of the lipid resuspension was loaded onto the column (4% lipid/sorbent (w/w) loading of the column). The neutral lipids were then eluted in 20 mL chloroform, which was collected in two in two volumes; an initial 5 mL elution and then the remaining 15 mL elution. The FFA were eluted in the same manner using 20 mL Diethyl Ether: Acetic Acid (98:2). The polar lipids were eluted in two washes, firstly 20 mL Methanol: Chloroform (6:1) and then 20 mL 0.05M sodium acetate in Methanol: Chloroform (6:1). 50 μL of each wash was loaded onto a HPTLC plate and run in Hexane: diethyl ether: acetic acid (70:30:1) for 10 cm, before being sprayed with primulin and visualized under UV. The first neutral lipid elution was transferred to a glass sample vial and was dried down under nitrogen gas and weighed. This is the Pure Neutral Lipid sample.

### Maillard Reaction of the Pure Neutral Lipid and Polar Lipid Fractions

In order to compare the aroma generated with the polar lipid and neutral lipid fractions, the following Maillard reactions were prepared:

TABLE 56
Maillard Reactions - for aroma assessment

| | | | | | | Actual |
|---|---|---|---|---|---|---|
| | | Sample | | | | Sample |
| Vial | | mass | | | Heating | Mass |
| no. | Sample | (mg) | | Matrix | conditions | (mg) |
| S1 | Plain matrix | 0 | mg | 2 mL | 140 degrees, | 0 |
| | control | | | matrix B | 45 minutes | |
| S2 | Neutral Lipid | 50 | mg | 2 mL | 140 degrees, | 50.2 |
| | Extract | | | matrix B | 45 minutes | |
| S3 | Polar Lipid | 50 | mg | 2 mL | 140 degrees, | 50.8 |
| | Extract | | | matrix B | 45 minutes | |

Volunteers sniffed the Maillard reactions and described the aromas in terms of pleasantness and meatiness. As shown in Table 57, the polar lipid generated more meaty and and more pleasant aromas than the Matrix only, or the neutral lipid.

TABLE 57
Aromas generated

| | | | Pleasantness | | | | | | | | |
|---|---|---|---|---|---|---|---|---|---|---|---|
| | | | | | | | | | | Stand- | |
| | | | Per- | Per- | Per- | Per- | Per- | Per- | | ard | |
| Sample | Test | | son | son | son | son | son | son | Aver- | Devi- | |
| Code | Type | Sample | 1 | 2 | 3 | 4 | 5 | 6 | age | ation | Total |
| 1 | Sniff | Plain | 4 | 4 | 3 | 4 | 1 | 2 | 3.00 | 1.26 | 18 |
| | | matrix | | | | | | | | | |
| | | control | | | | | | | | | |
| 2 | Sniff | Pure | 4 | 2 | 4 | 3 | 3 | 1 | 2.83 | 1.17 | 17 |
| | | Neutral | | | | | | | | | |
| | | Lipid | | | | | | | | | |
| | | Extract | | | | | | | | | |
| 3 | Sniff | Pure | 4 | 4 | 4 | 4 | 2 | 4 | 3.67 | 0.82 | 22 |
| | | Polar | | | | | | | | | |
| | | Lipid | | | | | | | | | |
| | | Extract | | | | | | | | | |

Aromas generated

| | | | Meatiness | | | | | | | | | |
|---|---|---|---|---|---|---|---|---|---|---|---|---|
| | | | | | | | | | | Stand- | | |
| | | | Per- | Per- | Per- | Per- | Per- | Per- | | ard | | |
| Sample | Test | | son | son | son | son | son | son | Aver- | Devi- | | |
| Code | Type | Sample | 1 | 2 | 3 | 4 | 5 | 6 | age | ation | Total | Total |
| 1 | Sniff | Plain | 2 | 3 | 2 | 2 | 2 | 2 | 2.17 | 0.41 | 13 | 31 |
| | | matrix | | | | | | | | | | |
| | | control | | | | | | | | | | |
| 2 | Sniff | Pure | 4 | 2 | 2 | 1 | 3 | 1 | 2.17 | 1.17 | 13 | 30 |
| | | Neutral | | | | | | | | | | |
| | | Lipid | | | | | | | | | | |
| | | Extract | | | | | | | | | | |
| 3 | Sniff | Pure | 3 | 3 | 3 | 3 | 2 | 3 | 2.83 | 0.41 | 17 | 39 |
| | | Polar | | | | | | | | | | |
| | | Lipid | | | | | | | | | | |
| | | Extract | | | | | | | | | | |

### Example 28. Assessment of Additional Mortierella Spp in Maillard Reaction

Additional Mortierella spp. isolates were identified and assessed in a Maillard reaction. One isolate (labelled Myu1) was identified as M. elongata and the other isolate (labelled S′2-1) was identified as M. exigua. The isolates were cultured and the resulting biomass analysed for fatty acid content in the lipid fraction. As shown in Table 57, ARA was present in an amount of about 33% of the total fatty acid content of the lipid of the Myu1 lipid, and in an amount of about 24% of the total fatty acid content of the S′2-1 lipid. The percentage oil by weight of the Myu1 isolate was 10.06%, while the percentage oil by weight of the S′2-1 isolate was 4.08%.

TABLE 57
Fatty acid composition of lipids from Mortierella isolates
Fatty acid composition (% mol)

| Sample | C14:0 | C16:0 | C16:1 | C18:0 | C18:1 | C18:liso | C18:2 | C18:3 | C20:0 | ARA |
|---|---|---|---|---|---|---|---|---|---|---|
| S'2-1 | 1.40 | 16.78 | 0.63 | 5.43 | 21.46 | 1.99 | 13.51 | 5.56 | 0.39 | 24.21 |
| Myu1 | 0.35 | 6.263 | 0 | 3.94 | 20.50 | 0.82 | 12.55 | 7.82 | 0.68 | 33.55 |

To assess the ability of each isolate to impart meaty aromas and flavours, a biomass equivalent to 50 mg dry matter of each isolate, as well as the M. alpina isolate, was weighed and transferred into a 20 mL glass vial. Matrix C (2 mL; defined in Table 53 above) was then added into each vial. A Matrix C only (i.e. no biomass) negative control was also included. The samples were then vigorously mixed for 2 min at room temp and subjected to heating at 140° C. for 45 min. After the heat treatment was completed, the samples were cooled down and tempered at 45° C.

A total of six participants (both male and female, aging from 25-65) were asked to sniff the M. alpina sample first and use it as the reference for testing other samples (at any order preferred by the participants). Between samples, the participants were requested to sniff the coffee to neutralize/clear the nose. The participants were requested to evaluate the aroma for the meatiness and pleasantness based on a five-point hedonic scale, with the higher score indicating the increased meatiness and pleasantness.

As shown in Table 58 below, each of the Mortierella isolates imparted a meaty aroma when heated in the presence of the Matrix components, above and beyond what was detected with the Matrix C only. While the M. alpina isolate imparted higher levels of meatiness, participants considered the M. elongata isolate to have the most pleasant aroma.

TABLE 58
Sensory assessment of Maillard reactions

| | Matrix C | M. alpina + | Myu1 + | S′2-1 + |
|---|---|---|---|---|
| | only | Matrix C | Matrix C | Matrix C |
| Pleasantness | 14.0 | 16.0 | 22.0 | 18.5 |
| Meatiness | 12.0 | 22.0 | 18.5 | 20.0 |
| Overall | 26.0 | 38.0 | 40.5 | 38.5 |

### Example 29. Comparison of M. Alpina Biomass and ARA Oil in Maillard Reaction

Previous studies indicated that ARA oil (i.e. TAG comprising about 40% ARA) produced a less meaty and less pleasant aroma when heated with a sugar and an amino acid than polar lipid containing ARA (see Example 7). To assess this further, the inventors compared the aromas generated by M. alpina biomass and ARA oil in Maillard reactions.

A set of 4 samples were prepared according to Table 59, using ARA oil from NuCheck Inc. (Cat #NC0632549). Sample C contained equivalent ARA to that in the biomass, while sample D contained 10% ARA oil. The samples were then vigorously mixed for 2 min at room temp and subjected to heating at 140° C. for 45 min. After the heat treatment was completed, the samples were cooled down and tempered at 45° C. throughout the sensory evaluation.

TABLE 59
Sample preparation

| Sample | Matrix C | M.Alpina biomass | ARA oil |
|---|---|---|---|
| A | 2 mL matrix C | NO biomass | No ARA oil |
| B | 2 mL matrix C | 200 g wet biomass | NO ARA oil |
| C | 2 mL matrix C | NO biomass | 13.1 mg ARA oil |
| D | 2 mL matrix C | NO biomass | 204.4 mg ARA oil |

A total of five participants (both male and female, aging from 25-65) were asked to sniff the samples in order of A to D. Between samples, the participants were requested to sniff the coffee to neutralize/clear the nose. The participants were requested to evaluate the aroma for the meatiness and pleasantness based on a five-point hedonic scale, with the higher score indicating the increased meatiness and pleasantness.

Samples having ARA oil added into the Maillard base were perceived as less pleasant and meaty (and more fatty/oily) compared to the samples having biomass, resulting in lower scores. This further supported the previous finding that polar lipid containing ARA produced more meaty and pleasant aromas than neutral lipid containing ARA.

TABLE 60
Sensory assessment

| | | Sample B | Sample C | Sample D |
|---|---|---|---|---|
| | Sample A | Matrix + | Matrix + | Matrix + |
| | Matrix only | biomass | ARA oil | ARA oil (10%) |
| Pleasantness | 14.0 | 17.0 | 13.0 | 12.0 |
| Meatiness | 13.0 | 19.0 | 11.5 | 13.0 |
| Total | 27.0 | 36.0 | 24.5 | 25.0 |

The present application claims priority from AU2021900593 filed 3 Mar. 2021, AU2021903366 filed 20 Oct. 2021, AU2021903367 filed 20 Oct. 2021, AU2021904195 filed 22 Dec. 2021 and AU2021904213 filed 22 Dec. 2021, the entire contents of each of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Adeyo et al. (2011) J Cell Biology, 192:1043-1055.
Al-Feel et al. (1992) Proc. Natl. Acad. Sci. USA. 89:4534-4538.
Aro et al. (1998) J Food Composition Anal 11:150-160.
Ashes et al. (1992) Lipids 27:629-631
Athenstaedt and Daum (1997). J. Bacteriol. 179:7611-7616.
Athenstaedt et al. (1999) J. Bacteriol. 181:1458-1463.
Bankaitis et al. (2007) Advances in enzyme regulation, 47:27.
Becker et al. (1991) Methods in Enzymology 194:182-7.
Belitz et al. (2009) Food chemistry (4th revised edition). Springer-Verlag, Berlin, Germany.
Beopoulos et al. (2008) App env micro. 74:7779-7789.
Bermingham et al. (2018) Animal Production Science 61:179-190
Bibikova et al. (2001) Mol. Cell. Biol. 21:289-287.
Bibikova et al. (2002) Genetics 161:1169-1175.
Bissett et al. (2016) GigaScience 5:21.
Bligh and Dyer (1959) Can. J. Biochem. Physiol. 37:911-918.
Boeke et al. (1984) Mol. Gen. Genet 197:345-47
Boselli et al. (2008) Meat Science 78:305-313.
Botha et al. (1998) Antonie van Leeuwenhoek 75:253-256.
Carsanba et al. (2020) Microorganisms 2020, 8:1054.
Carman and Gil-Soo (2011) Ann Rev Biochem 80:859-883.
Carman and Henry (1989) Annu. Rev. Biochem. 58:635-669
Carman and Henry (1999) Prog. Lipid Res. 38:361-399.
Carman and Zeimetz (1996) J. Biol. Chem. 271:13293-13296.
Carter and Kennedy (1966) J. Lipid Res. 7:678-683.
Casaregola et al. (2000) FEBS Lett. 487:95-100.
Celinska et al. (2017) Microb Biotechnol 10:450-455.
Certik et al. (1993) Acta Biotechnol 13:193-196.
Certo et al. (2012) Nat Methods 8:941-943.
Chang et al. (1998) J. Biol. Chem. 273:9829-9836.
Chang et al. (1998) J. Biol. Chem. 273:14933-14941.
Chisti and Moo-Young (1986). Enzyme and Microbial Technology 8:194-204.
Christie (1996) Lipid analysis. Trends in Food Science & Technology, 11:145.
Clancey et al. (1993) J Biol Chem, 268:24580-24590.
Cronan and Vagelos (1972) Biochim Biophys Acta. 265:25-60.
Dabirian et al. (2019). ACS synthetic biology, 8:1788-1800.
Daley et al. (2010). Nutrition Journal 2010, 9:10.
Dannenberger et al. (2006). J. Agric. Food Chem. 54:182-188.
Darvishi et al. (2011). New Biotechnol. 28:756-760.
Dashdorj et al. (2015). Eur Food Res technol 241:157-171.
De Castro and Priego-Capote (2010). J Chromatog A, 1217:2383-2389.
De Siervo (1969). J Bacteriology, 100:1342-1349.
De Virgilio et al. (1992) Yeast 8:1043-1051.
Dean-Johnson and Henry (1989). J. Biol. Chem. 264:1274-1283
Delhaize et al. (1999). J Biol Chem, 274:7082-7088.
Domergue et al. (2003) J. Biol. Chem. 278:35115-35126.
Domergue et al. (2005) Biochem. J. 389:483-490.
Doyon et al. (2010) Nat. Methods 7:459-460.
Dulermo et al. (2015). Biochim Biophys Acta (BBA)-Molecular and Cell Biology of
Lipids 1851:1107-1117.
Džugasová et al. (1998). Current genetics, 34:297-302.
Fakas (2017). Eng Life Sci 17:292-302.
Fakas et al. (2011). J Biol Chem, 286:1464-1474.
Farmer et al. (1990). J Sci Food Agric 53:505-523.
Feng et al. (2015). Metabolic engineering, 27:10-19.
Fernandez et al. (1986). J Bacteriol 166:779-786.
Ferreira et al. (2018). Proc. Natl. Acad. Sci. USA. 115:1262-1267.
Fickers et al. (2003). J Microbiol Methods 55:727-737.
Fischl et al. (1986). J Biol Chem, 261:3178-3183.
Friedlander et al. (2016). Biotechnol Biofuels 9:77
Fyrst et al. (1999). Biochemistry, 38 (18), 5864-5871.
Feiner (2006) Meat Products Handbook
Gietz et al. (1992) Nuc Acids Res. 27:69-74.
Gladkowski et al (2011) Food chemistry 126:1013-1018.
Gladkowski et al (2012) J Am Oil Chem Soc 89:179-182.
Groenen et al. (1993) Mol. Microbiol. 10:1057-1065.
Greenberg and Lopes (1996). Microbiol. Rev. 60:1-20
Gsell et al. (2013). PloS one, 8 (10), e77380.
Haft et al. (2005) Computational Biology 1 (6): e60
Hahn-Hägerdal et al. (2005). Microbial Cell Factories 4:31 doi: 10.1186/1475-2859-4-31.
Han et al. (2006). J. Biol. Chem. 281:9210-9218.
Han et al. (2008a). J. Biol. Chem. 283:20433-20442.
Han et al. (2008b). J. Biol. Chem. 283:20443-20453.
Harayama (1998). Trends Biotechnol. 16:76-82.
Hein and Hayen (2012). Metabolites 2012, 2, 254-267.
He et al. (2018). Biotechnol Biofuels 11:258.
Hegel et al (2011). J Supercrit Fluids. 58:68-78.
Helliwell et al. (1998). Genetics, 148:99-112.
Hiesinger et al. (1997). FEBS letters, 415:16-20.
Hildebrand and Law (1964). Biochem. 3:1304-1308.
Hjelmstad and Bell (1988). J. Biol. Chem. 263:19748-19757.
Hjelmstad and Bell (1991). J. Biol. Chem. 266:5094-5103.
Ho and Chen. (2008). Letters in Applied Microbiology 47:250-255.
Hoe et al. (1999) Emerg. Infect. Dis. 5:254-263.
Holic et al. (2012) Appl Microbiol Biotech. doi: 10.1007/s00253-012-3959-6
Hornstein et al. (1961). J. Food Sci. 26:581-586.
Hosaka et al. (1989). J. Biol. Chem. 264:2053-2059.
Huang et al. (1999). Lipids 34:649-659.
Ishino et al. (1987) J. Bacteriol. 169:5429-5433.
Itoh and Kaneko (1974). Yukagaku 23:350-354.
Ito et al. (1983) J. Bacteriol. 153:163-8
Janßen and Steinbüchel (2014). Biotechnology for biofuels, 7:1-26.
Jani and Lopes (2009). FEMS Yeast Research, 9:552-564.
Janssen et al. (2002) OMICS J. Integ. Biol. 6:23-33.
Kamisaka et al. (2015). Appl Microbiol Biotechnol 99:201-210.
Kanemasa et al. (1967). Biochim Biophys Acta 144:382-390.
Kanfer and Kennedy (1964). J. Biol. Chem. 239:1720-1726.
Kassab et al. (2019). Microbial cell factories, 18:1-10.
Kawai et al. (2010) Bioeng. Bugs 1:395-406.
Kim et al. (1996) Proc. Natl. Acad. Sci. USA 93:1156-1160
Kim et al. (1999). J. Biol. Chem. 274:14857-14866.
Kim et al. (2012) Genome Res. 22:1327-1333.
Klein et al. (2016). Molecular Biology of the Cell 27:2014-2024.
Klig and Henry (1984). Proc. Natl. Acad. Sci. USA 81:3816-3820.
Knutzon et al. (1998). J. Biol. Chem. 273:29360-29366.
Kodaki and Yamashita (1987). J. Biol. Chem. 262:15428-15435.
Kodaki and Yamashita (1989). European journal of biochemistry, 185:243-251.
Kooistra et al. (2004) Yeast 21:781-792
Kullenberg et al. (2012). Lipids in Health and Disease 11:3.
Larroude et al. (2018) Biotechnol Adv. 36:2150-2164.
Lee et al. (1994). J Biol Chem, 269:19725-19730.
Letts et al. (1983). Proc. Natl. Acad. Sci. USA 80:7279-7283.
Li et al. (2007). Biochim Biophys Acta (BBA)-Molecular and Cell Biology of
Lipids 1771:1246-1253.
Li et al. (2011) Biotechnol. Lett. 33:1823-1830.
Luke et al. (1996). Molecular and cellular biology, 16:2744-2755.
Mannion et al. (2018) J. Agric. Food Chem. 2019, 67, 499-506.
Marr and Ingraham (1962). J. Bacteriol 84:1260-1267.
Margetak et al. (2012). J. Lipids Article ID 543784, doi: 10.1155/2012/543784.
Masepohl et al. (1996) Biochim. Biophys. Acta 1307:26-30.
Mauersberger et al. (2001) J Bacteriol DOI: 10.1128/JB.183.17.5102-5109.2001
McConnell Smith et al. (2009) PNAS 106:5099-5104.
McGraw and Henry (1989) Genetics, 122:317-330.
Melton (1999). Current Status of Meat Flavour, Chapt 8 in Xiong et al., Quality Attributes of
Muscle Foods, Kluwer Academic/Plenum Publishers, New York
Meyer et al. (2003) Biochem. 42:9779-9788.
Meynier et al. (1998). JAOCS 75:1-7.
Michaelson et al. (1998). J Biol Chem, 273:19055-19059.
Min-Seok et al. (1996). J. Biochem. 120:1040-1047.
Mojica et al. (1995) Mol. Microbiol. 17:85-93.
Mojica et al. (2000) Mol. Microbiol. 36:244-246.
Mottram (1998). Food Chemistry 62:415-424.
Muller et al. (1998). Yeast 14:1267-1283.
Murray and Greenberg (2000). Mol. Microbiol. 36:651-661.
Nakata et al. (1989) J. Bacteriol. 171:3553-3556.
Napier et al. (2004). Patent publication No. WO2004/057001.
Needleman and Wunsch (1970) J. Mol. Biol. 48:443-453.
Nikawa and Yamashita (1984). Eur. J. Biochem. 143:251-256.
Nikawa et al. (1987) J. Biol. Chem. 262:4876.
Oh et al. (1997). J Biol Chem 272:17376-17384.
Oldham et al. (2001). Chem. Senses 26:529-531.
Orr-Weaver et al. (1981) PNAS USA 78:6354-58
Osman et al. (2010). EMBO J. 29:1976-1987.
Palacios and Wang (2005). J Am Oil Chem Soc 82:571-578.
Palmquist et al. (2005). Advances In Food and Nutrition Research 50:179-217.
Pascual et al. (2013). J Biol Chem, 288:35781-35792.
Patel et al. (2018) Molecules 23:1562.
Peter et al. (2017). PLOS ONE 12 (3): e0173739
Petrie et al. (2010) Biotechnol. 12:430-438.
Petrie et al. (2010a) Metab. Eng. 12:233-240.
Petrie et al. (2010b) Plant Methods 11:6:8.
Qi et al. (2002). FEBS Lett. 510:159-165.
Qiu et al. (2001) J. Biol. Chem. 276:31561-31566.
Ramirez et al. (2012) Nucleic Acids Res. 40:5560-5568.
Ratledge and Evans (1989). Lipids and their metabolism. In: Rose and Harrison (eds), The yeasts, 2nd edn. Academic, London, pp 367-455.
Ren et al. (2014). Molecular biology of the cell, 25:712-727.
Resconi et al. (2013) Molecules 2013, 18 (6), 6748-6781
Rong et al. (2015). eLife 2015; 4: e06557.
Ruiz-Lopez et al. (2012) Transgenic Res. 21:139-147.
Salimon et al. (2011). Chemistry Central Journal, 5 (1), 1-9.
Schüller et al. (1992). The EMBO journal, 11:107-114.
Schneiter et al. (2000). J Bacteriol, 182:3655-3660.
Shen et al. (1996). J Biol Chem, 271:789-795.
Shen and Dowhan (1997). J Biol Chem, 272:11215-11220.
Sreenivas et al. (1998). J Biol Chem, 273:16635-16638.
Sreenivas et al. (2001). J Biol Chem, 276:29915-29923.
Sreenivas and Carman (2003). J Biol Chem, 278:20673-20680.
Stukey et al. (1989). J Biol Chem 264:16537-16544.
Summers et al. (1988). Genetics, 120:909-922.
Sun et al. (2019). Food Science Nutr. 7:700-710.
Tamura et al. (2013). Cell metabolism, 17:709-718.
Tani and Kuge (2014). Yeast, 31:145-158.
Taormina et al. (2020). Nutrients 12, 2875.
Tezaki et al. (2017) Biochem Biophys Res Commun. 488:165-170
Tripathi et al. (2019). J Biol Chem, 294:19081-19098.
Trotter et al. (1993). J. Biol. Chem. 268:21416-21424.
Trotter and Voelker (1995). J Biol Chem, 270:6062-6070.
Tsukagoshi et al. (1987). Eur. J. Biochem. 169:477-486.
van Embden et al. (2000) J. Bacteriol. 182:2393-2401.
Wang et al. (2013). Planta 238:907-922.
Watanabe et al. (2004). Biosci Biotechnol Biochem 68:721-727.
Williams et al. (2008). Biochem. Biophys. Res. Commun. 374:620-624
Wongwathanarat et al. (1999). Microbiology 145:2939-2946.
Wang et al. (2012) Genome Res. 22:1316-1326.
Wood et al. (2003). Meat Science 66:21-32.
Wood et al. (2008). Meat Science 78:343-358.
Zank et al. (2002) Plant J. 31:255-268.
Zhang et al. (2014). Cell Mol Life Sci 71:3767-3778.
Zheng and Zou (2001). J. Biol. Chem. 276:41710-16
Zhou et al. (2007). Phytochemistry 68:785-796.
Zhou et al. (2008). Insect Mol. Biol. 17:667-676

1. A composition, comprising an amino acid or derivative, a sugar, and an extracted microbial lipid comprising esterified fatty acids in the form of either (i) polar lipid without any non-polar lipid, or (ii) polar lipid and non-polar lipid, the polar lipid being present in the extracted microbial lipid in a greater amount than the non-polar lipid, wherein
(a) the polar lipid of (i) and (ii) comprises a total fatty acid (TFA) content which comprises ω6 fatty acids, wherein at least some of the ω6 fatty acids are esterified in the form of phospholipids in the polar lipid, the ω6 fatty acids comprising arachidonic acid (ARA), dihomo-γ-linolenic acid (DGLA), and γ-linolenic acid (GLA), wherein ARA is present in an amount of about 10% to about 60% of the total fatty acid content of the polar lipid, DGLA is present in an amount of about 0.1% to about 5% of the total fatty acid content of the polar lipid and GLA is present in an amount of about 1% to about 10% of the total fatty acid content of the polar lipid,
(b) the polar lipid comprises a total saturated fatty acid content comprising palmitic acid and stearic acid, and
(c) the polar lipid comprises a total monounsaturated fatty acid content comprising oleic acid and palmitoleic acid (C16:1Δ9cis),
wherein when the composition is heated, one or more compounds which have a meat-associated flavour and/or aroma are produced.

2. The composition of claim 1, wherein:
ARA is present in an amount of about 20% to about 50% of the total fatty acid content of the polar lipid, DGLA is present in an amount of about 1% to about 5% of the total fatty acid content of the polar lipid and GLA is present in an amount of about 3% to about 10% of the total fatty acid content of the polar lipid; or
ARA is present in an amount of about 10% to about 20% of the total fatty acid content of the polar lipid, DGLA is present in an amount of about 0.5% to about 5% of the total fatty acid content of the polar lipid and GLA is present in an amount of about 3% to about 10% of the total fatty acid content of the polar lipid.

3-4. (canceled)

5. The composition of claim 1, wherein
the ω6 fatty acids are present in an amount of about 30% to about 70% of the total fatty acid content of the polar lipid.

6. The composition of claim 5, wherein:
the ω6 fatty acids are present in an amount of about 40% to about 70%, about 40% to about 60%, or about 50% to about 60% of the total fatty acid content of the polar lipid; and/or
ARA is present in an amount of about 20% to about 50% of the total fatty acid content of the polar lipid, DGLA is present in an amount of about 1% to about 5% of the total fatty acid content of the polar lipid and GLA is present in an amount of about 3% to about 10% of the total fatty acid content of the polar lipid.

7-8. (canceled)

9. The composition of claim 1, wherein ω3 fatty acids are either absent from the polar lipid or are present in a total amount of less than about 3% by weight of the TFA content of the polar lipid, and/or wherein the polar lipid lacks C16:2, C16:3ω3, EPA and DHA.

10. (canceled)

11. The composition of claim 1, wherein:
the phospholipids comprising the ω6 fatty acids comprise two, three, or all four of phosphatidylcholine (PC), phosphatidylethanolamine (PE), phosphatidylinositol (PI) and phosphatidylserine (PS), optionally one or more of phosphatidic acid (PA), phosphatidylglycerol (PG) and cardiolipin (Car), preferably comprising at least PC and PE or at least PC, PE, PS and PI, each comprising one or at least two or more of ARA, DGLA, and GLA;
the phospholipids comprising the ω6 fatty acids comprise phosphatidylcholine (PC) and phosphatidylethanolamine (PE), each comprising one or at least two or more of ARA, DGLA and GLA; and/or
the phospholipids comprising the ω6 fatty acids comprise phosphatidylcholine (PC) and phosphatidylethanolamine (PE), phosphatidylinositol (PI), phosphatidylserine (PS), and phosphatidic acid (PA), each comprising one or at least two or more of ARA, DGLA and GLA, wherein ARA is present in PC an amount of about 14% to about 20% of the total fatty acid content of the PC, ARA is present in PE an amount of about 15% to about 20% of the total fatty acid content of the PE, and ARA is present in PA an amount of about 15% to about 20% of the total fatty acid content of the PA.

12-14. (canceled)

15. The composition of claim 1, wherein the extracted microbial lipid is extracted fungal lipid or a eukaryotic microbial lipid, optionally selected from extracted yeast lipid, preferably a Saccharomyces cerevisiae, Yarrowia lipolytica, or Pichia pastoris lipid, and extracted Mortierella spp lipid.

16-17. (canceled)

18. The composition of claim 1, wherein at least one of the following apply:
at least one of EDA, DTA and DPA-ω3 is also present in the polar lipid;
the ratio of PC to PE or to phospholipids other than PC is less than 3:1, less than 2:1, less than 1.5:1, less than 1.25:1, less than 1:1, between 3:1 and 1:1, between 2:1 and 1:1, or between 3:1 and 0.5:1; and/or wherein:
the saturated fatty acid content of the polar lipid comprises one or more or all of lauric acid (C12:0), myristic acid (C14:0), a C15:0 fatty acid, C20:0, C22:0 and C24:0, preferably comprising C14:0 and C24:0 or C14:0, C15:0 and C24:0, more preferably comprising C14:0, C15:0 and C24:0 but not C20:0 and C22:0;
the stearic acid is present at a level of less than about 7% or less than about 6% or less than about 5%, preferably less than 4% or less than 3%, of the total fatty acid content of the polar lipid;
the polar lipid comprises myristic acid (C14:0) in an amount of less than about 2% by weight of the total fatty acid content of the polar lipid;
lauric acid and myristic acid are absent from the polar lipid, or lauric acid and/or myristic acid is present in the polar lipid, whereby the sum of the amounts of lauric acid and myristic acid in the polar lipid is less than about 2%, or less than about 1%, preferably less than about 0.5%, more preferably less than about 0.2%, of the total fatty acid content of the polar lipid;
C15:0 is absent from the polar lipid, or C15:0 is present in the polar lipid in an amount of less than about 3%, preferably less than about 2% or less than about 1%, of the total fatty acid content of the polar lipid;
palmitic acid is present in the polar lipid in an amount of about 10% to about 20% of the total fatty acid content of the polar lipid;
palmitoleic acid is present in the polar lipid in an amount of about 3% to about 45%, or about 3% to about 25%, or about 3% to about 20%, or about 3% to about 15%, of the total fatty acid content of the polar lipid;
oleic acid is present in the polar lipid in an amount of about 3% to about 60%, or about 3% to about 40%, or about 3% to about 25%, or about 20% to about 60%, of the total fatty acid content of the polar lipid;
vaccenic acid is absent from the polar lipid, or vaccenic acid is present in the polar lipid in an amount of less than about 2%, preferably less than about 1% or about 0.5%, of the total fatty acid content of the polar lipid;
linoleic acid is present in the polar lipid in an amount of about 3% to about 20% of the total fatty acid content of the polar lipid;
eicosadienoic acid is absent from the polar lipid, or eicosadienoic acid is present in the polar lipid in an amount of about 3% to about 12%, or about 3% to about 8%, or about 3% to about 6%, or less than about 3%, of the total fatty acid content of the polar lipid;
C20:0 and C22:0 are absent from the polar lipid, or C20:0 and/or C22:0 is present in the polar lipid, whereby the sum of the amounts of C20:0 and C22:0 in the polar lipid is less than about 1.0%, less than about 0.5%, preferably less than 0.2%, of the total fatty acid content of the polar lipid;
C24:0 is absent from the polar lipid, or C24:0 is present in the polar lipid in an amount of less than about 1.0%, less than 0.5%, preferably less than 0.3% or less than 0.2%, of the total fatty acid content of the polar lipid;
C17:1 is absent from the polar lipid, or C17:1 is present in the polar lipid in an amount of less than about 5%, preferably less than about 4% or less than about 3%, more preferably less than about 2% of the total fatty acid content of the polar lipid;
monounsaturated fatty acids which are C20 or C22 fatty acids are absent from the polar lipid, or C20:1 and/or C22:1 is present in the polar lipid, whereby the sum of the amounts of C20:1 and C22:1 in the polar lipid is less than about 1.0%, less than about 0.5%, preferably less than 0.2%, of the total fatty acid content of the polar lipid;
the content of ω6 fatty acids in the polar lipid which are (i) C20 or C22 fatty acids is about 5% to about 60%, preferably about 10% to about 60% of the total fatty acid content of the polar lipid, and/or (ii) ω6 fatty acids which have 3, 4 or 5 carbon-carbon double bonds, is about 5% to about 70%, preferably about 10% to about 70%, more preferably about 40% to about 70% or about 45% to about 70% or about 50% to about 70% of the total fatty acid content of the polar lipid;
C16:33 is absent from the polar lipid, or both C16:2 and C16:3 3 are absent from the polar lipid; and/or
the extracted microbial lipid comprises PC and/or lacks cyclopropane fatty acids, preferably which lacks C15:0c, C17:0c and C19:0c.

19-34. (canceled)

35. The composition of claim 1, wherein the extracted lipid is obtained from a genetically modified microbe, optionally wherein the genetically modified microbe has one or more genetic modification(s) which provide for
(a) synthesis of, or increased synthesis of, one or more ω6 fatty acids in the microbe,
(b) an increase in total fatty acid synthesis and/or accumulation in the microbe,
(c) an increase in total polar lipid synthesis and/or accumulation in the microbe,
(d) a decrease in triacylglycerol (TAG) synthesis and/or accumulation in the microbe, or an increase in TAG catabolism in the microbe, preferably an increase in TAG lipase activity,
(e) a reduction in catabolism of total fatty acids in the microbe,
or any combination thereof.

36-37. (canceled)

38. A composition, comprising an amino acid or derivative, a sugar, and an extracted Mortierella spp. lipid comprising esterified fatty acids in the form of either (i) polar lipid without any non-polar lipid, or (ii) polar lipid and non-polar lipid, the polar lipid being present in the extracted microbial lipid in a greater amount than the non-polar lipid.

39-40. (canceled)

41. The composition of claim 1, wherein the sugar, sugar alcohol, sugar acid, or sugar derivative is selected from ribose, xylose, glucose, fructose, sucrose, arabinose, glucose-6-phosphate, fructose-6-phosphate, fructose 1,6-diphosphate, inositol, maltose, molasses, altodextrin, glycogen, galactose, lactose, ribitol, gluconic acid and glucuronic acid, amylose, amylopectin, or any combination thereof, preferably wherein the sugar is ribose or xylose; and/or
wherein the amino acid or derivative thereof is selected from cysteine, cystine, a cysteine sulfoxide, allicin, selenocysteine, methionine, isoleucine, leucine, lysine, phenylalanine, threonine, tryptophan, 5-hydroxytryptophan, valine, arginine, histidine, alanine, asparagine, aspartate, glutamate, glutamine, glycine, proline, serine, tyrosine, or any combination thereof, preferably wherein the amino acid or derivative thereof is a sulfur-containing amino acid or derivative.

42. (canceled)

43. The composition of claim 1, which further comprises:
one or more fatty acids, esterified or non-esterified, from a source other than the extracted microbial lipid, cell or extract;
less than 5%, less than 10%, less than 15% or less than 20% (w/w or w/v) protein;
per gram of dry composition or slurry, or per ml of liquid composition, at least about 5 mg, at least about 10 mg, at least about 15 mg, at least about 20 mg, at least about 25 mg, or at least about 50 mg extracted microbial lipid; and/or
per gram of dry composition or slurry, or per ml of liquid composition, from about 10 mg to about 100 mg extracted microbial lipid or from about 15 mg to about 50 mg extracted microbial lipid.

44-47. (canceled)

48. A food, feedstuff or beverage comprising
(a) an ingredient which comprises the composition of claim 1 or the extracted microbial lipid as defined in claim 1, and at least one other food, feedstuff or beverage ingredient; or
(b) phospholipids and at least one other food, feedstuff or beverage ingredient, wherein the phospholipids are a product of a reaction between the extracted microbial lipid as defined in claim 1, an amino acid or derivative, and a sugar under conditions sufficient to produce at least two compounds which have a meat-associated flavour and/or aroma.

49-56. (canceled)

57. The food, feedstuff or beverage of claim 48, which has no components obtained from an animal.

58. (canceled)

59. A food or feedstuff, comprising at least two meat-associated flavour and/or aroma compounds derived from the extracted microbial lipid as defined in claim 1, or the composition of claim 1, wherein the food, feedstuff or beverage comprises a greater amount of the at least two compounds which have a meat-associated flavour and/or aroma than a corresponding food, feedstuff or beverage which was produced with a corresponding lipid or composition lacking the polar lipid comprising the ω6 fatty acid(s).

60. The food, feedstuff or beverage of claim 48, wherein the food, feedstuff or beverage is a meat substitute; and/or
applying heat to the food, feedstuff or beverage results in the production of one or more compound(s) which have a meat-associated flavour and/or aroma, preferably volatile compounds.

61. (canceled)

62. The food, feedstuff or beverage of claim 48, wherein applying heat to the food, feedstuff or beverage results in:
the production of two or more volatile compound(s) selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; 2,3,4,5-Tetramethylcyclopent-2-en-1-ol, 1-octanol, 2-heptanone, 3-octanone, 2,3-octanedione, 1-pentanol, 1-hexanol, 2-ethyl-1-hexanol, trans-2-octen-1-ol, 1-nonanol, 1,3-bis(1,1-dimethylethyl)-benzene, 2-octen-1-ol, adamantanol-like compound, hexanal, 2-pentyl furan, 1-octen-3-ol, 2-pentyl thiophene, and 1,3,5-thitriane;
the production of two or more volatile compound(s) selected from 2-heptanone, 3-octanone, 2,3-octanedione, 1-pentanol, 1-hexanol, 2-ethyl-1-hexanol, 1-octanol, trans-2-octen-1-ol and 1-nonanol;
the production of two or more volatile compound(s) selected from 1-pentanal, 3-octanone, 2-octen-1-ol, 1-nonanol and 1-octanol, and optionally 1,3-bis(1,1-dimethylethyl)-benzene; or
the production of two or more volatile compound(s) selected from 1,3-dimethyl benzene; p-xylene; ethylbenzene; 2-Heptanone; 2-pentyl furan; Octanal; 1,2-Octadecanediol; 2,4-diethyl-1-Heptanol; 2-Nonanone; Nonanal; 1-Octen-3-ol; 2-Decanone; 2-Octen-1-ol, (E)-; 2,4-dimethyl-Benzaldehyde; and 2,3,4,5-Tetramethylcyclopent-2-en-1-ol.

63-65. (canceled)

66. A method of producing a food, feedstuff or beverage, the method comprising combining
the composition of claim 1 or the extracted microbial lipid as defined in claim 1, optionally wherein the extracted microbial lipid has been heated at a temperature of at least about 100° C., at least about 120° C. or at least about 140° C., with a sugar, an amino acid or derivative, with at least one other food, feedstuff or beverage ingredient.

67-71. (canceled)

72. An isolated strain of Mortierella sp. selected from:
(a) yNI0125 deposited under V21/019953 on 12 Oct. 2021 at the National Measurement Institute Australia;
(b) yNI0126 deposited under V21/019951 on 12 Oct. 2021 at the National Measurement Institute Australia;
(c) yNI0127 deposited under V21/019952 on 12 Oct. 2021 at the National Measurement Institute Australia; and
(d) yNI0132 deposited under V21/019954 on 12 Oct. 2021 at the National Measurement Institute Australia.
